SEQUENCE LISTING (602 sequences)

SEQ ID NO: 1 - DNA, 5934 bp, Homo sapiens
atggctatgctgcctccacctggccctcagagcttcgtgcacttcaccaagcagagcctggccctgatcgagcagagaatcgccgagagaaagagcaaagagcccaaagaggaaaagaaggacgacgacgaggaagcccccaagcccagcagcgatctggaagctggaaagcagctgcccttcatctacggcgacatcccccctggcatggtgtccgagcctctggaagatctggacccctactacgccgacaagaaaaccttcatcgtgctgaacaagggcaagaccatcttcaggttcaacgccacccctgccctgtacatgctgagccccttcagccccctgagaagaatcagcatcaagatcctggtgcacagcctgttctccatgctgatcatgtgcaccatcctgaccaactgcatcttcatgaccatgaacaacccccccgactggaccaagaacgtggagtacaccttcaccggcatctacaccttcgagagcctcgtgaagattctggccaggggcttctgcgtgggcgagttcacattcctgagggacccctggaactggctggacttcgtcgtgatcgtgttcgcctacctgaccgagttcgtgaacctgggcaacgtgtccgccctgagaaccttcagagtgctgagagccctgaaaaccatcagcgtgatccccggcctgaaaacaatcgtgggcgccctgatccagagcgtgaagaaactgagcgacgtgatgatcctgaccgtgttctgcctgtctgtgttcgctctgatcggcctgcagctgttcatgggcaacctgaagcacaagtgcttccggaacagcctggaaaacaacgagacactggaatccatcatgaacaccctggaatccgaagaggatttccgcaagtacttctactacctggaaggcagcaaggacgccctgctgtgcggcttctctacagacagcggccagtgccccgagggctacacctgtgtgaagatcggcagaaaccccgactacggctacaccagcttcgataccttcagctgggccttcctggctctgttcagactgatgacccaggactactgggagaacctgtaccagcagaccctgagagccgctggcaagacctacatgatctttttcgtggtcgtgatcttcctgggcagcttctacctgatcaacctgatcctggctgtggtggccatggcttacgaggaacagaaccaggccaacatcgaagaggccaagcagaaagagctggaatttcagcagatgctggaccggctgaagaaagaacaggaagaggccgaggccattgccgccgctgctgccgagtacacatccatcaggcggagcagaatcatgggcctgagcgagagcagcagcgagacaagcaagctgagcagcaagtccgccaaagagagaagaaaccggcgcaagaagaagaaccagaagaagctgtccagcggcgaggaaaagggcgacgccgagaaactgtccaagagcgagtccgaggacagcatcagaagaaagtccttccacctgggcgtggaaggccacagaagggcccacgagaagagactgagcacccccaaccagagccctctgagcatcaggggcagcctgtttagcgccagaagatccagcagaacctccctgttcagcttcaagggcagaggcagagacatcggctccgagacagagttcgccgacgatgagcacagcatcttcggcgataacgagagcagacggggctctctgttcgtgccccacagaccccaggaaagaagaagcagcaacatcagccaggccagcagatccccccccatgctgcctgtgaacggcaagatgcacagcgccgtggactgcaacggcgtggtgtctctggtggatggcagatccgcactgatgctgcccaacggccagctgctgcctgagggcacaacaaatcaaatacacaagaaaaggcgttgtagttcctatctcctttcagaggatatgctgaacgaccccaacctgagacagagagccatgagcagagccagcatcctgaccaacaccgtggaagaactggaagagtccagacagaaatgccccccctggtggtacagattcgcccacaagtttctgatctggaactgcagcccctactggatcaagttcaagaagtgcatctacttcatcgtgatggaccccttcgtggacctggccatcaccatctgcatcgtgctgaacaccctgttcatggctatggaacaccaccccatgaccgaggaattcaagaacgtgctggccatcggcaacctggtgttcaccggcatcttcgccgccgagatggtgctgaagctgatcgccatggacccttacgagtacttccaagtgggctggaacatcttcgacagcctgatcgtgaccctgagcctggtggaactgttcctggccgacgtggaaggcctgagcgtgctgagaagcttcagactgctgagagtgttcaagctggccaagagctggcccaccctgaacatgctgatcaagatcatcggaaacagcgtgggcgccctgggcaacctgacactggtgctggctatcatcgtgttcatcttcgctgtcgtgggcatgcagctgttcggcaagagctacaaagaatgcgtgtgcaagatcaacgacgactgcaccctgcccagatggcacatgaacgatttcttccacagctttctgatcgtgttccgggtgctgtgcggcgagtggatcgagacaatgtgggactgcatggaagtggctggccaggccatgtgcctgattgtgtacatgatggtcatggtcatcgggaatctggtggtgctgaacctgtttctggccctgctgctgtccagcttctccagcgataacctgaccgccatcgaagaggaccccgacgccaacaacctgcagatcgccgtgaccagaatcaagaaaggcatcaactacgtgaagcagaccctgcgcgagttcatcctgaaggctttcagcaagaagcccaagatcagcagagagatcagacaggccgaggacctgaacaccaagaaagagaactacatcagcaaccacaccctggccgagatgagcaagggccataactttctgaaagagaaggacaagatctccggcttcggcagcagcgtggacaagcacctgatggaagatagcgacggccagagcttcatccacaacccctccctgaccgtgaccgtgcctatcgctcctggcgagagcgacctggaaaacatgaacgccgaggaactgagcagcgacagcgactccgagtacagcaaagtgcggctgaacagatccagcagcagcgagtgctccaccgtggacaatcctctgccaggcgagggcgaggaagctgaggctgagcctatgaacagcgacgagcccgaggcctgtttcaccgatggctgcgtgcggagattcagctgctgccaagtgaacatcgagagcggcaagggcaagatctggtggaatatcagaaagacctgctacaagatcgtggaacacagttggttcgagagctttattgtgctgatgatcctgctgtcctccggcgctctggccttcgaggacatctacatcgagcggaagaaaaccatcaagattatcctggaatacgccgataagatcttcacctacatcttcatcctggaaatgctgctgaagtggatcgcttacggctacaagacctacttcaccaacgcctggtgttggctggactttctgattgtggacgtgtccctcgtgactctggtggccaacaccctgggctacagcgatctgggccccatcaagagcctgagaaccctgagggctctgaggccactgagagccctgtccagattcgagggcatgagagtggttgtgaatgcactcataggagcaattccttccatcatgaatgtgctacttgtgtgtctgatcttctggctgatcttcagcatcatgggcgtgaacctgttcgccggcaagttctacgagtgcatcaacaccaccgacggcagcagattccccgccagccaggtgccaaacagatccgagtgcttcgccctgatgaacgtgtcccagaacgtgcggtggaagaacctgaaagtgaacttcgacaacgtgggcctgggctacctgagcctgctgcaggtggccacattcaagggctggaccatcattatgtacgccgccgtggacagcgtgaacgtggacaagcagcctaagtacgagtacagcctgtacatgtatatctacttcgtggtgtttattatcttcggcagcttcttcaccctgaatctgttcatcggcgtgatcatcgacaacttcaaccagcagaagaagaaactgggcggccaggacatcttcatgaccgaggaacagaaaaagtactacaacgccatgaagaagctgggcagcaagaagccccagaagcccatccccagacccggcaacaagatccagggctgcatcttcgacctcgtgaccaaccaggccttcgacatctccatcatggtgctgatctgcctgaacatggtcacaatgatggtggaaaaagagggccagagccagcacatgacagaggtgctgtactggatcaacgtggtgttcatcatcctgttcaccggcgagtgcgtgctgaagctgatctccctgcggcactactacttcaccgtgggctggaacatcttcgatttcgtggtcgtgatcatttctatcgtgggcatgttcctggccgacctgatcgagacatacttcgtgtcccccaccctgttcagagtgatcagactggccagaatcggcagaatcctgagactcgtgaagggcgccaagggcatcagaaccctgctgttcgctctgatgatgagcctgcccgccctgttcaatatcggcctgctgctgttcctcgtgatgttcatctacgccatcttcgggatgagcaacttcgcctacgtgaagaaagaggacggcatcaacgacatgttcaacttcgagacattcggcaacagcatgatctgtctgttccagatcaccaccagcgccggctgggatggactgctggctcctatcctgaacagcaagccccccgactgcgaccccaagaaggtgcaccctggcagcagcgtggaaggcgactgtggcaaccctagcgtgggcatcttctactttgtgtcctatatcatcattagctttctggtggtcgtgaacatgtacattgccgtgatcctggaaaacttcagcgtggccaccgaggaaagcaccgagcctctgagcgaggacgacttcgagatgttctacgaagtgtgggagaagttcgaccccgacgccacccagttcatcgagttcagcaagctgagcgacttcgctgccgccctggaccctcctctgctgatcgccaagcctaacaaggtgcagctgatcgctatggacctgcccatggtgtccggcgacagaatccactgcctggacatcctgtttgccttcaccaagagagtgctgggcgagagcggcgagatggacagcctgagaagccagatggaagaaagattcatgagcgccaaccccagcaaggtgtcctacgagcccatcaccacaaccctgaagagaaagcaggaagatgtgtccgccaccgtgatccagagagcctacagaagatacaggctgaggcagaatgtgaagaacatcagcagcatctacatcaaggacggcgacagggacgacgacctgctgaacaagaaagacatggccttcgataacgtgaacgagaacagctcccccgaaaagacagacgccaccagcagcaccacctccccacctagctacgactccgtgaccaagcccgacaaagagaagtacgagcaggacagaaccgagaaagaagataagggcaaggacagcaaagaaagcaagaagtga

SEQ ID NO: 2 - protein, 1977 aa, Homo sapiens
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVRRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 3 - protein, 8 aa, Homo sapiens
FMTMNNPP

SEQ ID NO: 4 - protein, 9 aa, Homo sapiens
LTEFVNLGN

SEQ ID NO: 5 - protein, 109 aa, Homo sapiens
MGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGK

SEQ ID NO: 6 - protein, 11 aa, Artificial Sequence
  KP1.1
CVELFLADVEG

SEQ ID NO: 7 - protein, 13 aa, Homo sapiens
MAMEHHPMTEEFK

SEQ ID NO: 8 - protein, 10 aa, Homo sapiens
VELFLADVEG

SEQ ID NO: 9 - protein, 54 aa, Homo sapiens
GKSYKECVCKINDDCTLPRWHMNDFFHSFLIVFRVLCGEWIETMWDCMEVAGQA

SEQ ID NO: 10 - protein, 11 aa, Artificial Sequence
  KP1.2
VELFLADVEGC

SEQ ID NO: 11 - protein, 15 aa, Homo sapiens
LAFEDIYIERKKTIK

SEQ ID NO: 12 - protein, 17 aa, Homo sapiens
VTLVANTLGYSDLGPIK

SEQ ID NO: 13 - protein, 88 aa, Homo sapiens
AGKFYECINTTDGSRFPASQVPNRSECFALMNVSQNVRWKNLKVNFDNVGLGYLSLLQVATFKGWTIIMYAAVDSVNVDKQPKYEYSL

SEQ ID NO: 14 - protein, 12 aa, Artificial Sequence
  KP1.3 (cyclic)
CVELFLADVEGC

SEQ ID NO: 15 - protein, 14 aa, Homo sapiens
MMVEKEGQSQHMTE

SEQ ID NO: 16 - protein, 20 aa, Homo sapiens
VGMFLADLIETYFVSPTLFR

SEQ ID NO: 17 - protein, 68 aa, Homo sapiens
AYVKKEDGINDMFNFETFGNSMICLFQITTSAGWDGLLAPILNSKPPDCDPKKVHPGSSVEGDCGNPS

SEQ ID NO: 18 - protein, 19 aa, Artificial Sequence
  KP2.1
CVGMFLADLIETYFVSPTL

SEQ ID NO: 19 - DNA, 5874 bp, Homo sapiens
atggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggtccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactctatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcatggtcaacatgtacattgcagtgattctggagaacttcaatgtggccacggaggagagcactgagcccctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctagtga

SEQ ID NO: 20 - protein, 1956 aa, Homo sapiens
MEFPIGSLETNNFRRFTPESLVEIEKQIAAKQGTKKAREKHREQKDQEEKPRPQLDLKACNQLPKFYGELPAELIGEPLEDLDPFYSTHRTFMVLNKGRTISRFSATRALWLFSPFNLIRRTAIKVSVHSWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALIHSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGKIYMIFFVLVIFLGSFYLVNLILAVVTMAYEEQNQATTDEIEAKEKKFQEALEMLRKEQEVLAALGIDTTSLHSHNGSPLTSKNASERRHRIKPRVSEGSTEDNKSPRSDPYNQRRMSFLGLASGKRRASHGSVFHFRSPGRDISLPEGVTDDGVFPGDHESHRGSLLLGGGAGQQGPLPRSPLPQPSNPDSRHGEDEHQPPPTSELAPGAVDVSAFDAGQKKTFLSAEYLDEPFRAQRAMSVVSIITSVLEELEESEQKCPPCLTSLSQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRTKQALCSFFSRSCPFPQPKAEPELVVKLPLSSSKAENHIAANTARGSSGGLQAPRGPRDEHSDFIANPTVWVSVPIAEGESDLDDLEDDGGEDAQSFQQEVIPKGQQEQLQQVERCGDHLTPRSPGTGTSSEDLAPSLGETWKDESVPQVPAEGVDDTSSSEGSTVDCLDPEEILRKIPELADDLEEPDDCFTEGCIRHCPCCKLDTTKSPWDVGWQVRKTCYRIVEHSWFESFIIFMILLSSGSLAFEDYYLDQKPTVKALLEYTDRVFTFIFVFEMLLKWVAYGFKKYFTNAWCWLDFLIVNISLISLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTKILGKINQFFVAVFTGECVMKMFALRQYYFTNGWNVFDFIVVVLSIASLIFSAILKSLQSYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIMVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTPCVPRAEEEAASLPDEGFVAFTANENCVLPDKSETASATSFPPSYESVTRGLSDRVNMRTSSSIQNEDEATSMELIAPGP

SEQ ID NO: 21 - protein, 8 aa, Homo sapiens
CMTRTDLP

SEQ ID NO: 22 - protein, 9 aa, Homo sapiens
VGTAIDLRG

SEQ ID NO: 23 - protein, 103 aa, Homo sapiens
KGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGK

SEQ ID NO: 24 - protein, 19 aa, Artificial Sequence
  KP2.2
VGMFLADLIETYFVSPTLC

SEQ ID NO: 25 - protein, 13 aa, Homo sapiens
MAMEHHGMSPTFE

SEQ ID NO: 26 - protein, 10 aa, Homo sapiens
LELGVAKKGS

SEQ ID NO: 27 - protein, 55 aa, Homo sapiens
GENYRNNRKNISAPHEDWPRWHMHDFFHSFLIVFRILCGEWIENMWACMEVGQKS

SEQ ID NO: 28 - protein, 20 aa, Artificial Sequence
  KP2.3 (cyclic)
CVGMFLADLIETYFVSPTLC

SEQ ID NO: 29 - protein, 15 aa, Homo sapiens
LAFEDYYLDQKPTVK

SEQ ID NO: 30 - protein, 17 aa, Homo sapiens
ISLTAKILEYSEVAPIK

SEQ ID NO: 31 - protein, 89 aa, Homo sapiens
AGKFWRCINYTDGEFSLVPLSIVNNKSDCKIQNSTGSFFWVNVKVNFDNVAMGYLALLQVATFKGWMDIMYAAVDSREVNMQPKWEDNV

SEQ ID NO: 32 - protein, 10 aa, Artificial Sequence
  KP5.1
CLTEFVNLGN

SEQ ID NO: 33 - protein, 14 aa, Homo sapiens
MMVETDDQSEEKTK

SEQ ID NO: 34 - protein, 22 aa, Homo sapiens
ASLIFSAILKSLQSYFSPTLFR

SEQ ID NO: 35 - protein, 67 aa, Homo sapiens
PHVRWEAGIDDMFNFQTFANSMLCLFQITTSAGWDGLLSPILNTGPPYCDPNLPNSNGTRGDCGSPA

SEQ ID NO: 36 - protein, 10 aa, Artificial Sequence
  KP5.2
LTEFVNLGNC

SEQ ID NO: 37 - DNA, 5376 bp, Homo sapiens
atggatgacagatgctacccagtaatctttccagatgagcggaatttccgccccttcacttccgactctctggctgcaattgagaagcggattgccatccaaaaggagaaaaagaagtctaaagaccagacaggagaagtaccccagcctcggcctcagcttgacctaaaggcctccaggaagttgcccaagctctatggcgacattcctcgtgagctcataggaaagcctctggaagacttggacccattctaccgaaatcataagacatttatggtgttaaacagaaagaggacaatctaccgcttcagtgccaagcatgccttgttcatttttgggcctttcaattcaatcagaagtttagccattagagtctcagtccattcattgttcagcatgttcattatcggcaccgttatcatcaactgcgtgttcatggctacagggcctgctaaaaacagcaacagtaacaatactgacattgcagagtgtgtcttcactgggatttatatttttgaagctttgattaaaatattggcaagaggtttcattctggatgagttttctttccttcgagatccatggaactggctggactccattgtcattggaatagcgattgtgtcatatattccaggaatcaccatcaaactattgcccctgcgtaccttccgtgtgttcagagctttgaaagcaatttcagtagtttcacgtctgaaggtcatcgtgggggccttgctacgctctgtgaagaagctggtcaacgtgattatcctcaccttcttttgcctcagcatctttgccctggtaggtcagcagctcttcatgggaagtctgaacctgaaatgcatctcgagggactgtaaaaatatcagtaacccggaagcttatgaccattgctttgaaaagaaagaaaattcacctgaattcaaaatgtgtggcatctggatgggtaacagtgcctgttccatacaatatgaatgtaagcacaccaaaattaatcctgactataattatacgaattttgacaactttggctggtcttttcttgccatgttccggctgatgacccaagattcctgggagaagctttatcaacagaccctgcgtactactgggctctactcagtcttcttcttcattgtggtcattttcctgggctccttctacctgattaacttaaccctggctgttgttaccatggcatatgaggagcagaacaagaatgtagctgcagagatagaggccaaggaaaagatgtttcaggaagcccagcagctgttaaaggaggaaaaggaggctctggttgccatgggaattgacagaagttcacttacttcccttgaaacatcatattttaccccaaaaaagagaaagctctttggtaataagaaaaggaagtccttctttttgagagagtctgggaaagaccagcctcctgggtcagattctgatgaagattgccaaaaaaagccacagctcctagagcaaaccaaacgactgtcccagaatctatcactggaccactttgatgagcatggagatcctctccaaaggcagagagcactgagtgctgtcagcatcctcaccatcaccatgaaggaacaagaaaaatcacaagagccttgtctcccttgtggagaaaacctggcatccaagtacctcgtgtggaactgttgcccccagtggctgtgcgttaagaaggtcctgagaactgtgatgactgacccgtttactgagctggccatcaccatctgcatcatcatcaacactgtcttcttggccatggagcatcacaagatggaggccagttttgagaagatgttgaatatagggaatttggttttcactagcatttttatagcagaaatgtgcctaaaaatcattgcgctcgatccctaccactactttcgccgaggctggaacatttttgacagcattgttgctcttctgagttttgcagatgtaatgaactgtgtacttcaaaagagaagctggccattcttgcgttccttcagagtgctcagggtcttcaagttagccaaatcctggccaactttgaacacactaattaagataatcggcaactctgtcggagcccttggaagcctgactgtggtcctggtcattgtgatctttattttctcagtagttggcatgcagctttttggccgtagcttcaattcccaaaagagtccaaaactctgtaacccgacaggcccgacagtctcatgtttacggcactggcacatgggggatttctggcactccttcctagtggtattccgcatcctctgcggggaatggatcgaaaatatgtgggaatgtatgcaagaagcgaatgcatcatcatcattgtgtgttattgtcttcatattgatcacggtgataggaaaacttgtggtgctcaacctcttcattgccttactgctcaattcctttagcaatgaggaaagaaatggaaacttagaaggagaggccaggaaaactaaagtccagttagcactggatcgattccgccgggctttttgttttgtgagacacactcttgagcatttctgtcacaagtggtgcaggaagcaaaacttaccacagcaaaaagaggtggcaggaggctgtgctgcacaaagcaaagacatcattcccctggtcatggagatgaaaaggggctcagagacccaggaggagcttggtatactaacctctgtaccaaagaccctgggcgtcaggcatgattggacttggttggcaccacttgcggaggaggaagatgacgttgaattttctggtgaagataatgcacagcgcatcacacaacctgagcctgaacaacaggcctatgagctccatcaggagaacaagaagcccacgagccagagagttcaaagtgtggaaattgacatgttctctgaagatgagcctcatctgaccatacaggatccccgaaagaagtctgatgttaccagtatactatcagaatgtagcaccattgatcttcaggatggctttggatggttacctgagatggttcccaaaaagcaaccagagagatgtttgcccaaaggctttggttgctgctttccatgctgtagcgtggacaagagaaagcctccctgggtcatttggtggaacctgcggaaaacctgctaccaaatagtgaaacacagctggtttgagagctttattatctttgtgattctgctgagcagtggggcactgatatttgaagatgttcaccttgagaaccaacccaaaatccaagaattactaaattgtactgacattatttttacacatatttttatcctggagatggtactaaaatgggtagccttcggatttggaaagtatttcaccagtgcctggtgctgccttgatttcatcattgtgattgtctctgtgaccaccctcattaacttaatggaattgaagtccttccggactctacgagcactgaggcctcttcgtgcgctgtcccagtttgaaggaatgaaggtggtggtcaatgctctcataggtgccatacctgccattctgaatgttttgcttgtctgcctcattttctggctcgtattttgtattctgggagtatacttcttttctggaaaatttgggaaatgcattaatggaacagactcagttataaattataccatcattacaaataaaagtcaatgtgaaagtggcaatttctcttggatcaaccagaaagtcaactttgacaatgtgggaaatgcttacctcgctctgctgcaagtggcaacatttaagggctggatggatattatatatgcagctgttgattccacagagaaagaacaacagccagagtttgagagcaattcactcggttacatttacttcgtagtctttatcatctttggctcattcttcactctgaatctcttcattggcgttatcattgacaacttcaaccaacagcagaaaaagttaggtggccaagacatttttatgacagaagaacagaagaaatactataatgcaatgaaaaaattaggatccaaaaaacctcaaaaacccattccacggcctctgaacaaatgtcaaggtctcgtgttcgacatagtcacaagccagatctttgacatcatcatcataagtctcattatcctaaacatgattagcatgatggctgaatcatacaaccaacccaaagccatgaaatccatccttgaccatctcaactgggtctttgtggtcatctttacgttagaatgtctcatcaaaatctttgctttgaggcaatactacttcaccaatggctggaatttatttgactgtgtggtcgtgcttctttccattgttagtacaatgatttctaccttggaaaatcaggagcacattcctttccctccgacgctcttcagaattgtccgcttggctcggattggccgaatcctgaggcttgtccgggctgcacgaggaatcaggactctcctctttgctctgatgatgtcgcttccttctctgttcaacattggtcttctactctttctgattatgtttatctatgccattctgggtatgaactggttttccaaagtgaatccagagtctggaatcgatgacatattcaacttcaagacttttgccagcagcatgctctgtctcttccagataagcacatcagcaggttgggattccctgctcagccccatgctgcgatcaaaagaatcatgtaactcttcctcagaaaactgccacctccctggcatagccacatcctactttgtcagttacattatcatctcctttctcattgttgtcaacatgtacattgctgtgattttagagaacttcaatacagccactgaagaaagtgaggaccctttgggtgaagatgactttgacatattttatgaagtgtgggaaaagtttgacccagaagcaacacaatttatcaaatattctgccctttctgactttgctgatgccttgcctgagcctttgcgtgtcgcaaagccaaataaatatcaatttctagtaatggacttgcccatggtgagtgaagatcgcctccactgcatggatattcttttcgccttcaccgctagggtactcggtggctctgatggcctagatagtatgaaagcaatgatggaagagaagttcatggaagccaatcctctcaagaagttgtatgaacccatagtcaccaccaccaagagaaaggaagaggaaagaggtgctgctattattcaaaaggcctttcgaaagtacatgatgaaggtgaccaagggtgaccaaggtgaccaaaatgacttggaaaacgggcctcattcaccactccagactctttgcaatggagacttgtctagctttggggtggccaagggcaaggtccactgtgactga

SEQ ID NO: 38 - protein, 1791 aa, Homo sapiens
MDDRCYPVIFPDERNFRPFTSDSLAAIEKRIAIQKEKKKSKDQTGEVPQPRPQLDLKASRKLPKLYGDIPRELIGKPLEDLDPFYRNHKTFMVLNRKRTIYRFSAKHALFIFGPFNSIRSLAIRVSVHSLFSMFIIGTVIINCVFMATGPAKNSNSNNTDIAECVFTGIYIFEALIKILARGFILDEFSFLRDPWNWLDSIVIGIAIVSYIPGITIKLLPLRTFRVFRALKAISVVSRLKVIVGALLRSVKKLVNVIILTFFCLSIFALVGQQLFMGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGLYSVFFFIVVIFLGSFYLINLTLAVVTMAYEEQNKNVAAEIEAKEKMFQEAQQLLKEEKEALVAMGIDRSSLTSLETSYFTPKKRKLFGNKKRKSFFLRESGKDQPPGSDSDEDCQKKPQLLEQTKRLSQNLSLDHFDEHGDPLQRQRALSAVSILTITMKEQEKSQEPCLPCGENLASKYLVWNCCPQWLCVKKVLRTVMTDPFTELAITICIIINTVFLAMEHHKMEASFEKMLNIGNLVFTSIFIAEMCLKIIALDPYHYFRRGWNIFDSIVALLSFADVMNCVLQKRSWPFLRSFRVLRVFKLAKSWPTLNTLIKIIGNSVGALGSLTVVLVIVIFIFSVVGMQLFGRSFNSQKSPKLCNPTGPTVSCLRHWHMGDFWHSFLVVFRILCGEWIENMWECMQEANASSSLCVIVFILITVIGKLVVLNLFIALLLNSFSNEERNGNLEGEARKTKVQLALDRFRRAFCFVRHTLEHFCHKWCRKQNLPQQKEVAGGCAAQSKDIIPLVMEMKRGSETQEELGILTSVPKTLGVRHDWTWLAPLAEEEDDVEFSGEDNAQRITQPEPEQQAYELHQENKKPTSQRVQSVEIDMFSEDEPHLTIQDPRKKSDVTSILSECSTIDLQDGFGWLPEMVPKKQPERCLPKGFGCCFPCCSVDKRKPPWVIWWNLRKTCYQIVKHSWFESFIIFVILLSSGALIFEDVHLENQPKIQELLNCTDIIFTHIFILEMVLKWVAFGFGKYFTSAWCCLDFIIVIVSVTTLINLMELKSFRTLRALRPLRALSQFEGMKVVVNALIGAIPAILNVLLVCLIFWLVFCILGVYFFSGKFGKCINGTDSVINYTIITNKSQCESGNFSWINQKVNFDNVGNAYLALLQVATFKGWMDIIYAAVDSTEKEQQPEFESNSLGYIYFVVFIIFGSFFTLNLFIGVIIDNFNQQQKKLGGQDIFMTEEQKKYYNAMKKLGSKKPQKPIPRPLNKCQGLVFDIVTSQIFDIIIISLIILNMISMMAESYNQPKAMKSILDHLNWVFVVIFTLECLIKIFALRQYYFTNGWNLFDCVVVLLSIVSTMISTLENQEHIPFPPTLFRIVRLARIGRILRLVRAARGIRTLLFALMMSLPSLFNIGLLLFLIMFIYAILGMNWFSKVNPESGIDDIFNFKTFASSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAAIIQKAFRKYMMKVTKGDQGDQNDLENGPHSPLQTLCNGDLSSFGVAKGKVHCD

SEQ ID NO: 39 - protein, 8 aa, Homo sapiens
FMATGPAK

SEQ ID NO: 40 - protein, 10 aa, Homo sapiens
VSYIPGITIK

SEQ ID NO: 41 - protein, 99 aa, Homo Sapiens
MGSLNLKCISRDCKNISNPEAYDHCFEKKENSPEFKMCGIWMGNSACSIQYECKHTKINPDYNYTNFDNFGWSFLAMFRLMTQDSWEKLYQQTLRTTGL

SEQ ID NO: 42 - protein, 11 aa, Artificial Sequence
  KP5.3 (cyclic)
CLTEFVNLGNC

SEQ ID NO: 43 - protein, 13 aa, Homo sapiens
LAMEHHKMEASFE

SEQ ID NO: 44 - protein, 12 aa, Homo sapiens
ADVMNCVLQKRS

SEQ ID NO: 45 - protein, 99 aa, Homo sapiens; identical to SEQ ID NO: 41

SEQ ID NO: 46 - protein, 17 aa, Artificial Sequence
  KP6.1
CVTLVANTLGYSDLGPI

SEQ ID NO: 47 - protein, 15 aa, Homo sapiens
LIFEDVHLENQPKIQ

SEQ ID NO: 48 - protein, 9 aa, Homo sapiens
TTLINMELK

SEQ ID NO: 49 - protein, 82 aa, Homo sapiens
SGKFGKCINGTDSVINYTIITNKSQCESGNFSWINQKVNFDNVGNAYLALLQVATFKGWMDIIYAAVDSTEKEQQPEFESNS

SEQ ID NO: 50 - protein, 17 aa, Artificial Sequence
  KP6.2
VTLVANTLGYSDLGPIC

SEQ ID NO: 51 - protein, 14 aa, Homo sapiens
MMAESYNQPKAMKS

SEQ ID NO: 52 - protein, 22 aa, Homo sapiens
VSTMISTLENQEHIPFPPTLFR

SEQ ID NO: 53 - protein, 59 aa, Homo sapiens
SKVNPESGIDDIFNFKTFASSMLCLFQISTSAGWDSLLSPMLRSKESCNSSSENCHLPG

SEQ ID NO: 54 - protein, 18 aa, Artificial Sequence
  KP6.3 (cyclic)
CVTLVANTLGYSDLGPIC

SEQ ID NO: 55 - DNA, 6030 bp, Homo sapiens
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 56 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 57 - protein, 8 aa, Homo sapiens
FMTMSNPP

SEQ ID NO: 58 - protein, 9 aa, Homo sapiens
VTEFVDLGN

SEQ ID NO: 59 - protein, 128 aa, Homo sapiens
MGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGK

SEQ ID NO: 60 - protein, 14 aa, Homo sapiens
TEFVNLGNVSALRT

SEQ ID NO: 61 - protein, 13 aa, Homo sapiens
MAMEHYPMTDHFN

SEQ ID NO: 62 - protein, 10 aa, Homo sapiens
VELGLANVEG

SEQ ID NO: 63 - protein, 54 aa, Homo sapiens
GKSYKDCVCKIASDCQLPRWHMNDFFHSFLIVFRVLCGEWIETMWDCMEVAGQA

SEQ ID NO: 64 - protein, 31 aa, Homo sapiens
TEFVNLGNVSALRTFRVLRALKTISVIPGLK

SEQ ID NO: 65 - protein, 15 aa, Homo sapiens
LAFEDIYIDQRKTIK

SEQ ID NO: 66 - protein, 17 aa, Homo sapiens
VSLTANALGYSELGAIK

SEQ ID NO: 67 - protein, 88 aa, Homo sapiens
AGKFYHCINTTTGDRFDIEDVNNHTDCLKLIERNETARWKNVKVNFDNVGFGYLSLLQVATFKGWMDIMYAAVDSRNVELQPKYEESL

SEQ ID NO: 68 - protein, 6 aa, Homo sapiens
NLGNVS

SEQ ID NO: 69 - protein, 14 aa, Homo sapiens
MMVETDDQSEYVTT

SEQ ID NO: 70 - protein, 20 aa, Homo sapiens
VGMFLAELIEKYFVSPTLFR

SEQ ID NO: 71 - protein, 68 aa, Homo sapiens
AYVKREVGIDDMFNFETFGNSMICLFQITTSAGWDGLLAPILNSKPPDCDPNKVNPGSSVKGDCGNPS

SEQ ID NO: 72 - protein, 1984 aa, Mus musculus
MAMLPPPGPQSFVHFTKQSLALIEQRISEEKAKGHKDEKKDDEEEGPKPSSDLEAGKQLPFIYGDIPPGMVSEPLEDLDPYYADKKTFIVLNKGKAIFRFNATPALYMLSPFSPLRRISIKILVHSLFSMLIMCTILTNCIFMTMSNPPDWTKNVEYTFTGIYTFESLIKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRKDLEQNETLESIMSTAESEEELKRYFYYLEGSKDALLCGFSTDSGQCPEGYECVTAGRNPDYGYTSFDTFGWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSLGRSRIMGLSESSSETSRLSSKSAKERRNRRKKKKQKLSSGEEKGDDEKLSKSGSEESIRKKSFHLGVEGHHRAREKRLSTPNQSPLSIRGSLFSARRSSRTSLFSFKGRGRDLGSETEFADDEHSIFGDNESRRGSLFVPHRPRERRSSNISQASRSPPVLPVNGKMHSAVDCNGVVSLVDGPSALMLPNGQLLPEVIIDKATSDDSGTTNQMRKKRLSSSYFLSEDMLNDPHLRQRAMSRASILTNTVEELEESRQKCPPWWYRFAHTFLIWNCSPYWIKFKKFIYFIVMDPFVDLAITICIVLNTLFMAMEHHPMTDEFKNVLAVGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINENCKLPRWHMNDFFHSFLIVFRVLCGEWIETMWDCMEVAGQTMCLIVYMMVMVIGNLVVLNLFLALLLSSFSSDNLTAIEEDTDANNLQIAVARIKRGINYVKQTLREFILKSFSKKPKGSKDTKRTADPNNKRENYISNRTLAEISKDHNFLKEKDKISGFSSSLDKSFMDENDYQSFIHNPSLTVTVPIAPGESDLENMNTEELSSDSDSDYSKERRNRSSSSECSTVDNPLPGEEEAEAEPINADEPEACFTDGCVRRFPCCQVNIDSGKGKVWWTIRKTCYRIVEHSWFESFIVLMILLSSGALAFEDIYIEKKKTIKIILEYADKIFTYIFILEMLLKWVAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECVNTTDGSRFSVSQVANRSECFALMNVSGNVRWKNLKVNFDNVGLGYLSLLQVATFKGWMDIMYAAVDSVNVNAQPIYEYNLYMYIYFVIFIIFGSFFTLNLFIGVIIDNFNQQKKKLGGQDIFMTEEQKKYYNAMKKLGSKKPQKPIPRPGNKFQGCIFDLVTNQAFDITIMVLICLNMVTMMVEKEGQTDYMSFVLYWINVVFIILFTGECVLKLISLRHYYFTVGWNIFDFVVVILSIVGMFLAEMIEKYFVSPTLFRVIRLARIGRILRLIKGAKGIRTLLFALMMSLPALFNIGLLLFLVMFIYAIFGMSNFAYVKKEAGINDMFNFETFGNSMICLFQITTSAGWDGLLAPILNSAPPDCDPKKVHPGSSVEGDCGNPSVGIFYFVSYIIISFLVVVNMYIAVILENFSVATEESTEPLSEDDFEMFYEVWEKFDPDATQFIEFCKLSDFAAALDPPLLIAKPNKVQLIAMDLPMVSGDRIHCLDILFAFTKRVLGESGEMDSLRSQMEERFMSANPSKVSYEPITTTLKRKQEDVSATIIQRAYRRYRLRQNVKNISSIYIKDGDRDDDLPNKEDIVFDNVNENSSPEKTDATASTISPPSYDSVTKPDQEKYETDKTEKEDKEKDESRK

SEQ ID NO: 73 - DNA, 8810 bp, Homo sapiens
atgctgttttctaacagacattgggtaccatcgaatgactgtcagaacagaaagctaaggcaaaggagggaggatgctgtggtcatcctttcttgtttttttcttctttaatgaggatagagcacatgtgagattttactttctactccagtaaaaattctgaagaattgcattggagactgttatattcaacacatacgtggattctgtgttatgatttacatttttctttatttcagcactttcttatgcaaggagctaaacagtgattaaaggagcaggatgaaaagatggcacagtcagtgctggtaccgccaggacctgacagcttccgcttctttaccagggaatcccttgctgctattgaacaacgcattgcagaagagaaagctaagagacccaaacaggaacgcaaggatgaggatgatgaaaatggcccaaagccaaacagtgacttggaagcaggaaaatctcttccatttatttatggagacattcctccagagatggtgtcagtgcccctggaggatctggacccctactatatcaataagaaaacgtttatagtattgaataaagggaaagcaatctctcgattcagtgccacccctgccctttacattttaactcccttcaaccctattagaaaattagctattaagattttggtacattctttattcaatatgctcattatgtgcacgattcttaccaactgtgtatttatgaccatgagtaaccctccagactggacaaagaatgtggagtatacctttacaggaatttatacttttgaatcacttattaaaatacttgcaaggggcttttgtttagaagatttcacatttttacgggatccatggaattggttggatttcacagtcattacttttgcatatgtgacagagtttgtggacctgggcaatgtctcagcgttgagaacattcagagttctccgagcattgaaaacaatttcagtcattccaggcctgaagaccattgtgggggccctgatccagtcagtgaagaagctttctgatgtcatgatcttgactgtgttctgtctaagcgtgtttgcgctaataggattgcagttgttcatgggcaacctacgaaataaatgtttgcaatggcctccagataattcttcctttgaaataaatatcacttccttctttaacaattcattggatgggaatggtactactttcaataggacagtgagcatatttaactgggatgaatatattgaggataaaagtcacttttattttttagaggggcaaaatgatgctctgctttgtggcaacagctcagatgcaggccagtgtcctgaaggatacatctgtgtgaaggctggtagaaaccccaactatggctacacgagctttgacacctttagttgggcctttttgtccttatttcgtctcatgactcaagacttctgggaaaacctttatcaactgacactacgtgctgctgggaaaacgtacatgatattttttgtgctggtcattttcttgggctcattctatctaataaatttgatcttggctgtggtggccatggcctatgaggaacagaatcaggccacattggaagaggctgaacagaaggaagctgaatttcagcagatgctcgaacagttgaaaaagcaacaagaagaagctcaggcggcagctgcagccgcatctgctgaatcaagagacttcagtggtgctggtgggataggagttttttcagagagttcttcagtagcatctaagttgagctccaaaagtgaaaaagagctgaaaaacagaagaaagaaaaagaaacagaaagaacagtctggagaagaagagaaaaatgacagagtccgaaaatcggaatctgaagacagcataagaagaaaaggtttccgtttttccttggaaggaagtaggctgacatatgaaaagagattttcttctccacaccagtccttactgagcatccgtggctcccttttctctccaagacgcaacagtagggcgagccttttcagcttcagaggtcgagcaaaggacattggctctgagaatgactttgctgatgatgagcacagcacctttgaggacaatgacagccgaagagactctctgttcgtgccgcacagacatggagaacggcgccacagcaatgtcagccaggccagccgtgcctccagggtgctccccatcctgcccatgaatgggaagatgcatagcgctgtggactgcaatggtgtggtctccctggtcgggggcccttctaccctcacatctgctgggcagctcctaccagagggcacaactactgaaacagaaataagaaagagacggtccagttcttatcatgtttccatggatttattggaagatcctacatcaaggcaaagagcaatgagtatagccagtattttgaccaacaccatggaagaacttgaagaatccagacagaaatgcccaccatgctggtataaatttgctaatatgtgtttgatttgggactgttgtaaaccatggttaaaggtgaaacaccttgtcaacctggttgtaatggacccatttgttgacctggccatcaccatctgcattgtcttaaatacactcttcatggctatggagcactatcccatgacggagcagttcagcagtgtactgtctgttggaaacctggtcttcacagggatcttcacagcagaaatgtttctcaagataattgccatggatccatattattactttcaagaaggctggaatatttttgatggttttattgtgagccttagtttaatggaacttggtttggcaaatgtggaaggattgtcagttctccgatcattccggctgctccgagttttcaagttggcaaaatcttggccaactctaaatatgctaattaagatcattggcaattctgtgggggctctaggaaacctcaccttggtattggccatcatcgtcttcatttttgctgtggtcggcatgcagctctttggtaagagctacaaagaatgtgtctgcaagatttccaatgattgtgaactcccacgctggcacatgcatgactttttccactccttcctgatcgtgttccgcgtgctgtgtggagagtggatagagaccatgtgggactgtatggaggtcgctggccaaaccatgtgccttactgtcttcatgatggtcatggtgattggaaatctagtggttctgaacctcttcttggccttgcttttgagttccttcagttctgacaatcttgctgccactgatgatgataacgaaatgaataatctccagattgctgtgggaaggatgcagaaaggaatcgattttgttaaaagaaaaatacgtgaatttattcagaaagcctttgttaggaagcagaaagctttagatgaaattaaaccgcttgaagatctaaataataaaaaagacagctgtatttccaaccataccaccatagaaataggcaaagacctcaattatctcaaagacggaaatggaactactagtggcataggcagcagtgtagaaaaatatgtcgtggatgaaagtgattacatgtcatttataaacaaccctagcctcactgtgacagtaccaattgctgttggagaatctgactttgaaaatttaaatactgaagaattcagcagcgagtcagatatggaggaaagcaaagagaagctaaatgcaactagttcatctgaaggcagcacggttgatattggagctcccgccgagggagaacagcctgaggttgaacctgaggaatcccttgaacctgaagcctgttttacagaagactgtgtacggaagttcaagtgttgtcagataagcatagaagaaggcaaagggaaactctggtggaatttgaggaaaacatgctataagatagtggagcacaattggttcgaaaccttcattgtcttcatgattctgctgagcagtggggctctggcctttgaagatatatacattgagcagcgaaaaaccattaagaccatgttagaatatgctgacaaggttttcacttacatattcattctggaaatgctgctaaagtgggttgcatatggttttcaagtgtattttaccaatgcctggtgctggctagacttcctgattgttgatgtctcactggttagcttaactgcaaatgccttgggttactcagaacttggtgccatcaaatccctcagaacactaagagctctgaggccactgagagctttgtcccggtttgaaggaatgagggttgttgtaaatgctcttttaggagccattccatctatcatgaatgtacttctggtttgtctgatcttttggctaatattcagtatcatgggagtgaatctctttgctggcaagttttaccattgtattaattacaccactggagagatgtttgatgtaagcgtggtcaacaactacagtgagtgcaaagctctcattgagagcaatcaaactgccaggtggaaaaatgtgaaagtaaactttgataacgtaggacttggatatctgtctctacttcaagtagccacgtttaagggatggatggatattatgtatgcagctgttgattcacgaaatgtagaattacaacccaagtatgaagacaacctgtacatgtatctttattttgtcatctttattatttttggttcattctttaccttgaatcttttcattggtgtcatcatagataacttcaaccaacagaaaaagaagtttggaggtcaagacatttttatgacagaagaacagaagaaatactacaatgcaatgaaaaaactgggttcaaagaaaccacaaaaacccatacctcgacctgctaacaaattccaaggaatggtctttgattttgtaaccaaacaagtctttgatatcagcatcatgatcctcatctgccttaacatggtcaccatgatggtggaaaccgatgaccagagtcaagaaatgacaaacattctgtactggattaatctggtgtttattgttctgttcactggagaatgtgtgctgaaactgatctctcttcgttactactatttcactattggatggaatatttttgattttgtggtggtcattctctccattgtaggaatgtttctggctgaactgatagaaaagtattttgtgtcccctaccctgttccgagtgatccgtcttgccaggattggccgaatcctacgtctgatcaaaggagcaaaggggatccgcacgctgctctttgctttgatgatgtcccttcctgcgttgtttaacatcggcctccttcttttcctggtcatgttcatctacgccatctttgggatgtccaattttgcctatgttaagagggaagttgggatcgatgacatgttcaactttgagacctttggcaacagcatgatctgcctgttccaaattacaacctctgctggctgggatggattgctagcacctattcttaatagtggacctccagactgtgaccctgacaaagatcaccctggaagctcagttaaaggagactgtgggaacccatctgttgggattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcgacccagtttatagagtttgccaaactttctgattttgcagatgccctggatcctcctcttctcatagcaaaacccaacaaagtccagctcattgccatggatctgcccatggtgagtggtgaccggatccactgtcttgacatcttatttgcttttacaaagcgtgttttgggtgagagtggagagatggatgcccttcgaatacagatggaagagcgattcatggcatcaaacccctccaaagtctcttatgagcccattacgaccacgttgaaacgcaaacaagaggaggtgtctgctattattatccagagggcttacagacgctacctcttgaagcaaaaagttaaaaaggtatcaagtatatacaagaaagacaaaggcaaagaatgtgatggaacacccatcaaagaagatactctcattgataaactgaatgagaattcaactccagagaaaaccgatatgacgccttccaccacgtctccaccctcgtatgatagtgtgaccaaaccagaaaaagaaaaatttgaaaaagacaaatcagaaaaggaagacaaagggaaagatatcagggaaagtaaaaagtaaaaagaaaccaagaattttccattttgtgatcaattgtttacagcccgtgatggtgatgtgtttgtgtcaacaggactcccacaggaggtctatgccaaactgactgtttttacaaatgtatacttaaggtcagtgcctataacaagacagagacctctggtcagcaaactggaactcagtaaactggagaaatagtatcgatgggaggtttctattttcacaaccagctgacactgctgaagagcagaggcgtaatggctactcagacgataggaaccaatttaaaggggggagggaagttaaatttttatgtaaattcaacatgtgacacttgataatagtaattgtcaccagtgtttatgttttaactgccacacctgccatatttttacaaaacgtgtgctgtgaatttatcacttttctttttaattcacaggttgtttactattatatgtgactatttttgtaaatgggtttgtgtttggggagagggattaaagggagggaattctacatttctctattgtattgtataactggatatattttaaatggaggcatgctgcaattctcattcacacataaaaaaatcacatcacaaaagggaagagtttacttcttgtttcaggatgtttttagatttttgaggtgcttaaatagctattcgtatttttaaggtgtctcatccagaaaaaatttaatgtgcctgtaaatgttccatagaatcacaagcattaaagagttgttttatttttacataacccattaaatgtacatgtatatatgtatatatgtatatgtgcgtgtatatacatatatatgtatacacacatgcacacacagagatatacacataccattacattgtcattcacagtcccagcagcatgactatcacatttttgataagtgtcctttggcataaaataaaaatatcctatcagtcctttctaagaagcctgaattgaccaaaaaacatccccaccaccactttataaagttgattctgctttatcctgcagtattgtttagccatcttctgctcttggtaaggttgacatagtatatgtcaatttaaaaaataaaagtctgctttgtaaatagtaattttacccagtggtgcatgtttgagcaaacaaaaatgatgatttaagcacactacttattgcatcaaatatgtaccacagtaagtatagtttgcaagctttcaacaggtaatatgatgtaattggttccattatagtttgaagctgtcactgctgcatgtttatcttgcctatgctgctgtatcttattccttccactgttcagaagtctaatatgggaagccatatatcagtggtaaagtgaagcaaattgttctaccaagacctcattcttcatgtcattaagcaataggttgcagcaaacaaggaagagcttcttgctttttattcttccaaccttaattgaacactcaatgatgaaaagcccgactgtacaaacatgttgcaagctgcttaaatctgtttaaaatatatggttagagttttctaagaaaatataaatactgtaaaaagttcattttattttatttttcagccttttgtacgtaaaatgagaaattaaaagtatcttcaggtggatgtcacagtcactattgttagtttctgttcctagcacttttaaattgaagcacttcacaaaataagaagcaaggactaggatgcagtgtaggtttctgcttttttattagtactgtaaacttgcacacatttcaatgtgaaacaaatctcaaactgagttcaatgtttatttgctttcaatagtaatgccttatcattgaaagaggcttaaagaaaaaaaaaatcagctgatactcttggcattgcttgaatccaatgtttccacctagtctttttattcagtaatcatcagtcttttccaatgtttgtttacacagatagatcttattgacccatatggcactagaactgtatcagatataatatgggatcccagctttttttcctctcccacaaaaccaggtagtgaagttatattaccagttacagcaaaatactttgtgtttcacaagcaacaataaatgtagattctttatactgaagctattgacttgtagtgtgttggtgaaatgcatgcaggaaaatgctgttaccataaagaacggtaaaccacattacaatcaagccaaaagaataaaggtttcgcttttgtttttgtatttaattgttgtctttgtttctatctttgaaatgccatttaaaggtagatttctatcatgtaaaaataatctatctgaaaaacaaatgtaaagaacacacattaattactataattcatctttcaattttttcatggaatggaagttaattaagaagagtgtattggataactactttaatattggccaaaaagctagatatggcatcaggtagactagtggaaagttacaaaaattaataaaaaattgactaacattttaaaaaaaaaaaaaaaaaa

SEQ ID NO: 74 - protein, 2005 aa, Homo sapiens
MAQSVLVPPGPDSFRFFTRESLAAIEQRIAEEKAKRPKQERKDEDDENGPKPNSDLEAGKSLPFIYGDIPPEMVSVPLEDLDPYYINKKTFIVLNKGKAISRFSATPALYILTPFNPIRKLAIKILVHSLFNMLIMCTILTNCVFMTMSNPPDWTKNVEYTFTGIYTFESLIKILARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMLEQLKKQQEEAQAAAAAASAESRDFSGAGGIGVFSESSSVASKLSSKSEKELKNRRKKKKQKEQSGEEEKNDRVRKSESEDSIRRKGFRFSLEGSRLTYEKRFSSPHQSLLSIRGSLFSPRRNSRASLFSFRGRAKDIGSENDFADDEHSTFEDNDSRRDSLFVPHRHGERRHSNVSQASRASRVLPILPMNGKMHSAVDCNGVVSLVGGPSTLTSAGQLLPEGTTTETEIRKRRSSSYHVSMDLLEDPTSRQRAMSIASILTNTMEELEESRQKCPPCWYKFANMCLIWDCCKPWLKVKHLVNLVVMDPFVDLAITICIVLNTLFMAMEHYPMTEQFSSVLSVGNLVFTGIFTAEMFLKIIAMDPYYYFQEGWNIFDGFIVSLSLMELGLANVEGLSVLRSFRLLRVFKLAKSWPTLNMLIKIIGNSVGALGNLTLVLAIIVFIFAVVGMQLFGKSYKECVCKISNDCELPRWHMHDFFHSFLIVFRVLCGEWIETMWDCMEVAGQTMCLTVFMMVMVIGNLVVLNLFLALLLSSFSSDNLAATDDDNEMNNLQIAVGRMQKGIDFVKRKIREFIQKAFVRKQKALDEIKPLEDLNNKKDSCISNHTTIEIGKDLNYLKDGNGTTSGIGSSVEKYVVDESDYMSFINNPSLTVTVPIAVGESDFENLNTEEFSSESDMEESKEKLNATSSSEGSTVDIGAPAEGEQPEVEPEESLEPEACFTEDCVRKFKCCQISIEEGKGKLWWNLRKTCYKIVEHNWFETFIVFMILLSSGALAFEDIYIEQRKTIKTMLEYADKVFTYIFILEMLLKWVAYGFQVYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINYTTGEMFDVSVVNNYSECKALIESNQTARWKNVKVNFDNVGLGYLSLLQVATFKGWMDIMYAAVDSRNVELQPKYEDNLYMYLYFVIFIIFGSFFTLNLFIGVIIDNFNQQKKKFGGQDIFMTEEQKKYYNAMKKLGSKKPQKPIPRPANKFQGMVFDFVTKQVFDISIMILICLNMVTMMVETDDQSQEMTNILYWINLVFIVLFTGECVLKLISLRYYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSGPPDCDPDKDHPGSSVKGDCGNPSVGIFFFVSYIIISFLVVVNMYIAVILENFSVATEESAEPLSEDDFEMFYEVWEKFDPDATQFIEFAKLSDFADALDPPLLIAKPNKVQLIAMDLPMVSGDRIHCLDILFAFTKRVLGESGEMDALRIQMEERFMASNPSKVSYEPITTTLKRKQEEVSAIIIQRAYRRYLLKQKVKKVSSIYKKDKGKECDGTPIKEDTLIDKLNENSTPEKTDMTPSTTSPPSYDSVTKPEKEKFEKDKSEKEDKGKDIRESKK

SEQ ID NO: 75 - protein, 8 aa, Homo Sapiens
FMTMSNPP

SEQ ID NO: 76 - protein, 9 aa, Homo Sapiens
VTEFVDLGN

SEQ ID NO: 77 - protein, 129 aa, Homo Sapiens
MGNLRNKCLQWPPDNSSFEINITSFFNNSLDGNGTTFNRTVSIFNWDEYIEDKSHFYFLEGQNDALLCGNSSDAGQCPEGYICVKAGRNPNYGYTSFDTFSWAFLSLFRLMTQDFWENLYQLTLRAAGK

SEQ ID NO: 78 - protein, 1958 aa, Mus musculus
MEFPFGSVGTTNFRRFTPESLAEIEKQIAAHRAAKKGRPKQRGQKDKSEKPRPQLDLKACNQLPRFYGELPAELVGEPLEDLDPFYSTHRTFIVLDKSRTISRFSATWALWLFSPFNLIRRTAIKVSVHSWFSIFITVTILVNCVCMTRTDLPEKLEYAFTVVYTFEALIKILARGFCLNEFTYLRDPWNWLDFSVITLAYVGAAIDLRGISGLRTFRVLRALKTVSVIPGLKVIVGALIHSVRKLADVTILTVFCLSVFALVGLQLFKGNLKNKCIKNGTDPHKADNLSSEMAGDIFIKPGTTDPLLCGNGSDAGHCPNDYVCRKTSDNPDFNYTSFDSFAWAFLSLFRLMTQDSWERLYQQTLRASGKMYMVFFVLVIFLGSFYLVNLILAVVTMAYEEQSQATIAEIEAKEKKFKEALEVLQKEQEVLAALGIDTTSLYSHNGSPLAPKNANERRPRVKSRMSEGSTDDNRSLQSDPYNQRRMSFLGLSSGRRRASHSSVFHFRAPSQDVSFPDGILDDGVFHGDQESRRSSILLGRGAGQAGPLPRSPLPQSPNPGPRRGEEGQRGVPTGELATGAPEGPALDAAGQKNFLSADYLNEPFRAQRAMSVVSIMTSVIEELEESKLKCPPCLISLAQKYLIWECCPKWKKFKMVLFELVTDPFAELTITLCIVVNTVFMAMEHYPMTDAFDAMLQAGNIVFTVFFTMEMAFKIIAFDPYYYFQKKWNIFDCVIVTVSLLELSTSKKGSLSVLRTFRLLRVFKLAKSWPTLNMLIKIIGNSVGALGNLTFILAIIVFIFALVGKQLLSENYGCRRDGISVWNGERLRWHMCDFFHSFLVVFRILCGEWIENMWVCMEVSQDYICLTLFLTVMVLGNLVVLNLFIALLLNSFSADNLTAPEDDGEVNNLQVALARIQVFGHRASRAITSYIRSHCRLRWPKVETQLGMKPPLTSCKAENHIATDAVNAAVGNLAKPALGGPKENHGDFITDPNVWVSVPIAEGESDLDELEEDVEHASQSSWQEESPKGQQELLQQVQKCEDHQAARSPPSGMSSEDLAPYLGERWQREESPRVPAEGVDDTSSSEGSTVDCPDPEEILRKIPELAEELDEPDDCFPEGCTRRCPCCKVNTSKFPWATGWQVRKTCYRIVEHSWFESFIIFMILLSSGALAFEDNYLEEKPRVKSVLEYTDRVFTFIFVFEMLLKWVAYGFKKYFTNAWCWLDFLIVNISLTSLIAKILEYSDVASIKALRTLRALRPLRALSRFEGMRVVVDALVGAIPSIMNVLLVCLIFWLIFSIMGVNLFAGKFSRCVDTRSNPFSVVNSTFVTNKSDCYNQNNTGHFFWVNVKVNFDNVAMGYLALLQVATFKGWMDIMYAAVDSRDINSQPNWEESLYMYLYFVVFIIFGGFFTLNLFVGVIIDNFNQQKKKIRGQDIFMTEEQKKYYNAMKKLGSKKPQKPIPRPLNKYQGFVFDIVTRQAFDIIIMALICLNMITMMVETDNQSEEKTKVLGRINQFFVAVFTGECVMKMFALRQYYFTNGWNVFDFIVVILSISSLLFSAILSSLESYFSPTLLRVIRLARIGRILRLIRAAKGIRTLLFALMMSLPALFNIGLLLFLVMFIYSIFGMASFANVIDEAGIDDMFNFKTFGNSMLCLFQITTSAGWDGLLSPILNTGPPYCDPNRPNSNGSKGNCGSPAVGILFFTTYIIISFLIVVNMYIAVILENFNVATEESTEPLSEDDFDMFYETWEKFDPEATQFIAFSALSDFADTLSGPLRIPKPNQNILIQMDLPLVPGDKIHCLDILFAFTKNVLGESGELDSLKTNMEEKFMATNLSKASYEPIATTLRCKQEDISATIIQKAYRNYMLQRSLMLSNPLHVPRAEEDGVSLPREGYVTFMANDNGGLPDKSETASATSFPPSYDSVTRGLSDRANISTSSSMQNEDEVTAKEGKSPGPQ

SEQ ID NO: 79 - protein, 13 aa, Homo Sapiens
MAMEHYPMTEQFS

SEQ ID NO: 80 - protein, 10 aa, Homo Sapiens
MELGLANVEG

SEQ ID NO: 81 - protein, 54 aa, Homo Sapiens
GKSYKECVCKISNDCELPRWHMHDFFHSFLIVFRVLCGEWIETMWDCMEVAGQT

SEQ ID NO: 82 - protein, 1765 aa, Mus musculus
MEERYYPVIFPDERNFRPFTFDSLAAIEKRITIQKEKKKSKDKAATEPQPRPQLDLKASRKLPKLYGDVPPDLIAKPLEDLDPFYKDHKTFMVLNKKRTIYRFSAKRALFILGPFNPIRSFMIRISVHSVFSMFIICTVIINCMFMANNSSVDSRPSSNIPEYVFIGIYVLEAVIKILARGFIVDEFSYLRDPWNWLDFIVIGTAIAPCFLGNKVNNLSTLRTFRVLRALKAISVISGLKVIVGALLRSVKKLVDVMVLTLFCLSIFALVGQQLFMGILSQKCIKDDCGPNAFSNKDCFVKENDSEDFIMCGNWLGRRSCPDGSTCNKTTFNPDYNYTNFDSFGWSFLAMFRVMTQDSWEKLYRQILRTSGIYFVFFFVVVIFLGSFYLLNLTLAVVTMAYEEQNRNVAAETEAKEKMFQEAQQLLREEKEALVAMGIDRTSLNSLQASSFSPKKRKFFGSKTRKSFFMRGSKTARASASDSEDDASKNPQLLEQTKRLSQNLPVELFDEHVDPLHRQRALSAVSILTITMQEQEKSQEPCFPCGKNLASKYLVWECSPPWLCIKKVLQTIMTDPFTELAITICIIVNTVFLAMEHHNMDNSLKDILKIGNWVFTGIFIAEMCLKIIALDPYHYFRHGWNIFDSIVALVSLADVLFHKLSKNLSFLASLRVLRVFKLAKSWPTLNTLIKIIGHSVGALGNLTVVLTIVVFIFSVVGMRLFGAKFNKTCSTSPESLRRWHMGDFYHSFLVVFRILCGEWIENMWECMQEMEGSPLCVIVFVLIMVVGKLVVLNLFIALLLNSFSNEEKDGNPEGETRKTKVQLALDRFSRAFYFMARALQNFCCKRCRRQNSPKPNEATESFAGESRDTATLDTRSWKEYDSEMTLYTGQAGAPLAPLAKEEDDMECCGECDASPTSQPSEEAQACDLPLKTKRLPSPDDHGVEMEVFSEEDPNLTIQSARKKSDAASMLSECSTIDLNDIFRNLQKTVSPQKQPDRCFPKGLSCIFLCCKTIKKKSPWVLWWNLRKTCYQIVKHSWFESFIIFVILLSSGALIFEDVNLPSRPQVEKLLKCTDNIFTFIFLLEMILKWVAFGFRKYFTSAWCWLDFLIVVVSGLSLTNLPNLKSFRNLRALRPLRALSQFEGMKVVVNALMSAIPAILNVLLVCLIFWLIFCILGVNFFSGKFGRCINGTDINKYFNASNVPNQSQCLVSNYTWKVPNVNFDNVGNAYLALLQVATYKGWLDIMNAAVDSRGKDEQPAFEANLYAYLYFVVFIIFGSFFTLNLFIGVIIDNFNQQQKKLGGQDIFMTEEQKKYYNAMKKLGTKKPQKPIPRPLNKCQAFVFDLVTSQVFDVIILGLIVTNMIIMMAESEGQPNEVKKIFDILNIVFVVIFTVECLIKVFALRQHYFTNGWNLFDCVVVVLSIISTLVSGLENSNVFPPTLFRIVRLARIGRILRLVRAARGIRTLLFALMMSLPSLFNIGLLLFLVMFIYAIFGMNWFSKVKRGSGIDDIFNFDTFSGSMLCLFQITTSAGWDALLNPMLESKASCNSSSQESCQQPQIAIVYFVSYIIISFLIVVNMYIAVILENFNTATEESEDPLGEDDFEIFYEIWEKFDPEATQFIQYSSLSDFADALPEPLRVAKPNRFQFLMMDLPMVMGDRLHCMDVLFAFTTRVLGNSSGLDTMKAMMEEKFMEANPFKKLYEPIVTTTKRKEEEECAAVIQRAYRRHMEKMIKLKLKGRSSSSLQVFCNGDLSSLDVPKIKVHCD

SEQ ID NO: 83 - protein, 15 aa, Homo Sapiens
LAFEDIYIEQRKTIK

SEQ ID NO: 84 - protein, 17 aa, Homo Sapiens
VSLTANALGYSELGAIK

SEQ ID NO: 85 - protein, 88 aa, Homo Sapiens
AGKFYHCINYTTGEMFDVSVVNNYSECKALIESNQTARWKNVKVNFDNVGLGYLSLLQVATFKGWMDIMYAAVDSRNVELQPKYEDNL

SEQ ID NO: 86 - protein, 6 aa, Homo Sapiens
VSYIPG

SEQ ID NO: 87 - protein, 14 aa, Homo Sapiens
MMVETDDQSQEMTN

SEQ ID NO: 88 - protein, 20 aa, Homo Sapiens
VGMFLAELIEKYFVSPTLFR

SEQ ID NO: 89 - protein, 68 aa, Homo Sapiens
AYVKREVGIDDMFNFETFGNSMICLFQITTSAGWDGLLAPILNSGPPDCDPDKDHPGSSVKGDCGNPS

SEQ ID NO: 90 - protein, 8 aa, Homo Sapiens
TMISTLEN

SEQ ID NO: 91 - DNA, 6003 bp, Homo Sapiens
atggcacaggcactgttggtacccccaggacctgaaagcttccgcctttttactagagaatctcttgctgctatcgaaaaacgtgctgcagaagagaaagccaagaagcccaaaaaggaacaagataatgatgatgagaacaaaccaaagccaaatagtgacttggaagctggaaagaaccttccatttatttatggagacattcctccagagatggtgtcagagcccctggaggacctggatccctactatatcaataagaaaacttttatagtaatgaataaaggaaaggcaattttccgattcagtgccacctctgccttgtatattttaactccactaaaccctgttaggaaaattgctatcaagattttggtacattctttattcagcatgcttatcatgtgcactattttgaccaactgtgtatttatgaccttgagcaaccctcctgactggacaaagaatgtagagtacacattcactggaatctatacctttgagtcacttataaaaatcttggcaagagggttttgcttagaagattttacgtttcttcgtgatccatggaactggctggatttcagtgtcattgtgatggcatatgtgacagagtttgtggacctgggcaatgtctcagcgttgagaacattcagagttctccgagcactgaaaacaatttcagtcattccaggtttaaagaccattgtgggggccctgatccagtcggtaaagaagctttctgatgtgatgatcctgactgtgttctgtctgagcgtgtttgctctcattgggctgcagctgttcatgggcaatctgaggaataaatgtttgcagtggcccccaagcgattctgcttttgaaaccaacaccacttcctactttaatggcacaatggattcaaatgggacatttgttaatgtaacaatgagcacatttaactggaaggattacattggagatgacagtcacttttatgttttggatgggcaaaaagaccctttactctgtggaaatggctcagatgcaggccagtgtccagaaggatacatctgtgtgaaggctggtcgaaaccccaactatggctacacaagctttgacacctttagctgggctttcctgtctctatttcgactcatgactcaagactactgggaaaatctttaccagttgacattacgtgctgctgggaaaacatacatgatattttttgtcctggtcattttcttgggctcattttatttggtgaatttgatcctggctgtggtggccatggcctatgaggagcagaatcaggccaccttggaagaagcagaacaaaaagaggccgaatttcagcagatgctcgaacagcttaaaaagcaacaggaagaagctcaggcagttgcggcagcatcagctgcttcaagagatttcagtggaataggtgggttaggagagctgttggaaagttcttcagaagcatcaaagttgagttccaaaagtgctaaagaatggaggaaccgaaggaagaaaagaagacagagagagcaccttgaaggaaacaacaaaggagagagagacagctttcccaaatccgaatctgaagacagcgtcaaaagaagcagcttccttttctccatggatggaaacagactgaccagtgacaaaaaattctgctcccctcatcagtctctcttgagtatccgtggctccctgttttccccaagacgcaatagcaaaacaagcattttcagtttcagaggtcgggcaaaggatgttggatctgaaaatgactttgctgatgatgaacacagcacatttgaagacagcgaaagcaggagagactcactgtttgtgccgcacagacatggagagcgacgcaacagtaacgttagtcaggccagtatgtcatccaggatggtgccagggcttccagcaaatgggaagatgcacagcactgtggattgcaatggtgtggtttccttggtgggtggaccttcagctctaacgtcacctactggacaacttcccccagagggcaccaccacagaaacggaagtcagaaagagaaggttaagctcttaccagatttcaatggagatgctggaggattcctctggaaggcaaagagccgtgagcatagccagcattctgaccaacacaatggaagaacttgaagaatctagacagaaatgtccgccatgctggtatagatttgccaatgtgttcttgatctgggactgctgtgatgcatggttaaaagtaaaacatcttgtgaatttaattgttatggatccatttgttgatcttgccatcactatttgcattgtcttaaataccctctttatggccatggagcactaccccatgactgagcaattcagtagtgtgttgactgtaggaaacctggtctttactgggattttcacagcagaaatggttctcaagatcattgccatggatccttattactatttccaagaaggctggaatatctttgatggaattattgtcagcctcagtttaatggagcttggtctgtcaaatgtggagggattgtctgtactgcgatcattcagactgcttagagttttcaagttggcaaaatcctggcccacactaaatatgctaattaagatcattggcaattctgtgggggctctaggaaacctcaccttggtgttggccatcatcgtcttcatttttgctgtggtcggcatgcagctctttggtaagagctacaaagaatgtgtctgcaagatcaatgatgactgtacgctcccacggtggcacatgaacgacttcttccactccttcctgattgtgttccgcgtgctgtgtggagagtggatagagaccatgtgggactgtatggaggtcgctggccaaaccatgtgccttattgttttcatgttggtcatggtcattggaaaccttgtggttctgaacctctttctggccttattgttgagttcatttagctcagacaaccttgctgctactgatgatgacaatgaaatgaataatctgcagattgcagtaggaagaatgcaaaagggaattgattatgtgaaaaataagatgcgggagtgtttccaaaaagccttttttagaaagccaaaagttatagaaatccatgaaggcaataagatagacagctgcatgtccaataatactggaattgaaataagcaaagagcttaattatcttagagatgggaatggaaccaccagtggtgtaggtactggaagcagtgttgaaaaatacgtaatcgatgaaaatgattatatgtcattcataaacaaccccagcctcaccgtcacagtgccaattgctgttggagagtctgactttgaaaacttaaatactgaagagttcagcagtgagtcagaactagaagaaagcaaagagaaattaaatgcaaccagctcatctgaaggaagcacagttgatgttgttctaccccgagaaggtgaacaagctgaaactgaacccgaagaagaccttaaaccggaagcttgttttactgaaggatgtattaaaaagtttccattctgtcaagtaagtacagaagaaggcaaagggaagatctggtggaatcttcgaaaaacctgctacagtattgttgagcacaactggtttgagactttcattgtgttcatgatccttctcagtagtggtgcattggcctttgaagatatatacattgaacagcgaaagactatcaaaaccatgctagaatatgctgacaaagtctttacctatatattcattctggaaatgcttctcaaatgggttgcttatggatttcaaacatatttcactaatgcctggtgctggctagatttcttgatcgttgatgtttctttggttagcctggtagccaatgctcttggctactcagaactcggtgccatcaaatcattacggacattaagagctttaagacctctaagagccttatcccggtttgaaggcatgagggtggttgtgaatgctcttgttggagcaattccctctatcatgaatgtgctgttggtctgtctcatcttctggttgatctttagcatcatgggtgtgaatttgtttgctggcaagttctaccactgtgttaacatgacaacgggtaacatgtttgacattagtgatgttaacaatttgagtgactgtcaggctcttggcaagcaagctcggtggaaaaacgtgaaagtaaactttgataatgttggcgctggctatcttgcactgcttcaagtggccacatttaaaggctggatggatattatgtatgcagctgttgattcacgagatgttaaacttcagcctgtatatgaagaaaatctgtacatgtatttatactttgtcatctttatcatctttgggtcattcttcactctgaatctattcattggtgtcatcatagataacttcaaccagcagaaaaagaagtttggaggtcaagacatctttatgacagaggaacagaaaaaatattacaatgcaatgaagaaacttggatccaagaaacctcagaaacccatacctcgcccagcaaacaaattccaaggaatggtctttgattttgtaaccagacaagtctttgatatcagcatcatgatcctcatctgcctcaacatggtcaccatgatggtggaaacggatgaccagggcaaatacatgaccctagttttgtcccggatcaacctagtgttcattgttctgttcactggagaatttgtgctgaagctcgtctccctcagacactactacttcactataggctggaacatctttgactttgtggtggtgattctctccattgtaggtatgtttctggctgagatgatagaaaagtattttgtgtcccctaccttgttccgagtgatccgtcttgccaggattggccgaatcctacgtctgatcaaaggagcaaaggggatccgcacgctgctctttgctttgatgatgtcccttcctgcgttgtttaacatcggcctcctgctcttcctggtcatgtttatctatgccatctttgggatgtccaactttgcctatgttaaaaaggaagctggaattgatgacatgttcaactttgagacctttggcaacagcatgatctgcttgttccaaattacaacctctgctggctgggatggattgctagcacctattcttaatagtgcaccacccgactgtgaccctgacacaattcaccctggcagctcagttaagggagactgtgggaacccatctgttgggattttcttttttgtcagttacatcatcatatccttcctggttgtggtgaacatgtacatcgcggtcatcctggagaacttcagtgttgctactgaagaaagtgcagagcccctgagtgaggatgactttgagatgttctatgaggtttgggaaaagtttgatcccgatgcgacccagtttatagagttctctaaactctctgattttgcagctgccctggatcctcctcttctcatagcaaaacccaacaaagtccagcttattgccatggatctgcccatggtcagtggtgaccggatccactgtcttgatattttatttgcctttacaaagcgtgttttgggtgagagtggagagatggatgcccttcgaatacagatggaagacaggtttatggcatcaaacccctccaaagtctcttatgagcctattacaaccactttgaaacgtaaacaagaggaggtgtctgccgctatcattcagcgtaatttcagatgttatcttttaaagcaaaggttaaaaaatatatcaagtaactataacaaagaggcaattaaagggaggattgacttacctataaaacaagacatgattattgacaaactaaatgggaactccactccagaaaaaacagatgggagttcctctaccacctctcctccttcctatgatagtgtaacaaaaccagacaaggaaaagtttgagaaagacaaaccagaaaaagaaagcaaaggaaaagaggtcagagaaaatcaaaagtaa

SEQ ID NO: 92 - protein, 2000 aa, Homo Sapiens
MAQALLVPPGPESFRLFTRESLAAIEKRAAEEKAKKPKKEQDNDDENKPKPNSDLEAGKNLPFIYGDIPPEMVSEPLEDLDPYYINKKTFIVMNKGKAIFRFSATSALYILTPLNPVRKIAIKILVHSLFSMLIMCTILTNCVFMTLSNPPDWTKNVEYTFTGIYTFESLIKILARGFCLEDFTFLRDPWNWLDFSVIVMAYVTEFVDLGNVSALRTFRVLRALKTISVIPGLKTIVGALIQSVKKLSDVMILTVFCLSVFALIGLQLFMGNLRNKCLQWPPSDSAFETNTTSYFNGTMDSNGTFVNVTMSTFNWKDYIGDDSHFYVLDGQKDPLLCGNGSDAGQCPEGYICVKAGRNPNYGYTSFDTFSWAFLSLFRLMTQDYWENLYQLTLRAAGKTYMIFFVLVIFLGSFYLVNLILAVVAMAYEEQNQATLEEAEQKEAEFQQMLEQLKKQQEEAQAVAAASAASRDFSGIGGLGELLESSSEASKLSSKSAKEWRNRRKKRRQREHLEGNNKGERDSFPKSESEDSVKRSSFLFSMDGNRLTSDKKFCSPHQSLLSIRGSLFSPRRNSKTSIFSFRGRAKDVGSENDFADDEHSTFEDSESRRDSLFVPHRHGERRNSNVSQASMSSRMVPGLPANGKMHSTVDCNGVVSLVGGPSALTSPTGQLPPEGTTTETEVRKRRLSSYQISMEMLEDSSGRQRAVSIASILTNTMEELEESRQKCPPCWYRFANVFLIWDCCDAWLKVKHLVNLIVMDPFVDLAITICIVLNTLFMAMEHYPMTEQFSSVLTVGNLVFTGIFTAEMVLKIIAMDPYYYFQEGWNIFDGIIVSLSLMELGLSNVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQTMCLIVFMLVMVIGNLVVLNLFLALLLSSFSSDNLAATDDDNEMNNLQIAVGRMQKGIDYVKNKMRECFQKAFFRKPKVIEIHEGNKIDSCMSNNTGIEISKELNYLRDGNGTTSGVGTGSSVEKYVIDENDYMSFINNPSLTVTVPIAVGESDFENLNTEEFSSESELEESKEKLNATSSSEGSTVDVVLPREGEQAETEPEEDLKPEACFTEGCIKKFPFCQVSTEEGKGKIWWNLRKTCYSIVEHNWFETFIVFMILLSSGALAFEDIYIEQRKTIKTMLEYADKVFTYIFILEMLLKWVAYGFQTYFTNAWCWLDFLIVDVSLVSLVANALGYSELGAIKSLRTLRALRPLRALSRFEGMRVVVNALVGAIPSIMNVLLVCLIFWLIFSIMGVNLFAGKFYHCVNMTTGNMFDISDVNNLSDCQALGKQARWKNVKVNFDNVGAGYLALLQVATFKGWMDIMYAAVDSRDVKLQPVYEENLYMYLYFVIFIIFGSFFTLNLFIGVIIDNFNQQKKKFGGQDIFMTEEQKKYYNAMKKLGSKKPQKPIPRPANKFQGMVFDFVTRQVFDISIMILICLNMVTMMVETDDQGKYMTLVLSRINLVFIVLFTGEFVLKLVSLRHYYFTIGWNIFDFVVVILSIVGMFLAEMIEKYFVSPTLFRVIRLARIGRILRLIKGAKGIRTLLFALMMSLPALFNIGLLLFLVMFIYAIFGMSNFAYVKKEAGIDDMFNFETFGNSMICLFQITTSAGWDGLLAPILNSAPPDCDPDTIHPGSSVKGDCGNPSVGIFFFVSYIIISFLVVVNMYIAVILENFSVATEESAEPLSEDDFEMFYEVWEKFDPDATQFIEFSKLSDFAAALDPPLLIAKPNKVQLIAMDLPMVSGDRIHCLDILFAFTKRVLGESGEMDALRIQMEDRFMASNPSKVSYEPITTTLKRKQEEVSAAIIQRNFRCYLLKQRLKNISSNYNKEAIKGRIDLPIKQDMIIDKLNGNSTPEKTDGSSSTTSPPSYDSVTKPDKEKFEKDKPEKESKGKEVRENQK

SEQ ID NO: 93 - protein, 8 aa, Homo Sapiens
FMTLSNPP

SEQ ID NO: 94 - protein, 9 aa, Homo Sapiens
VTEFVDLGN

SEQ ID NO: 95 - protein, 129 aa, Homo Sapiens
MGNLRNKCLQWPPSDSAFETNTTSYFNGTMDSNGTFVNVTMSTFNWKDYIGDDSHFYVLDGQKDPLLCGNGSDAGQCPEGYICVKAGRNPNYGYTSFDTFSWAFLSLFRLMTQDYWENLYQLTLRAAGK

SEQ ID NO: 96 - protein, 17 aa, Homo Sapiens
VSTMISTLENQEHIPFP

SEQ ID NO: 97 - protein, 13 aa, Homo Sapiens
MAMEHYPMTEQFS

SEQ ID NO: 98 - protein, 10 aa, Homo Sapiens
MELGLSNVEG

SEQ ID NO: 99 - protein, 54 aa, Homo Sapiens
GKSYKECVCKINDDCTLPRWHMNDFFHSFLIVFRVLCGEWIETMWDCMEVAGQT

SEQ ID NO: 100 - protein, 15 aa, Homo Sapiens
VGMFLADLIETYFVS

SEQ ID NO: 101 - protein, 15 aa, Homo Sapiens
LAFEDIYIEQRKTIK

SEQ ID NO: 102 - protein, 17 aa, Homo Sapiens
VSLVANALGYSELGAIK

SEQ ID NO: 103 - protein, 85 aa, Homo Sapiens
AGKFYHCVNMTTGNMFDISDVNNLSDCQALGKQARWKNVKVNFDNVGAGYLALLQVATFKGWMDIMYAAVDSRDVKLQPVYEENL

SEQ ID NO: 104 - protein, 9 aa, Homo Sapiens
LIFSAILKS

SEQ ID NO: 105 - protein, 14 aa, Homo Sapiens
MMVETDDQGKYMTL

SEQ ID NO: 106 - protein, 20 aa, Homo Sapiens
VGMFLAEMIEKYFVSPTLFR

SEQ ID NO: 107 - protein, 68 aa, Homo Sapiens
AYVKKEAGIDDMFNFETFGNSMICLFQITTSAGWDGLLAPILNSAPPDCDPDTIHPGSSVKGDCGNPS

SEQ ID NO: 108 - protein, 16 aa, Homo Sapiens
ASLIFSAILKSLQSYF

SEQ ID NO: 109 - DNA, 5511 bp, Homo Sapiens
atggccagaccatctctgtgcaccctggtgcctctgggccctgagtgcttgcgccccttcacccgggagtcactggcagccatagaacagcgggcggtggaggaggaggcccggctgcagcggaataagcagatggagattgaggagcccgaacggaagccacgaagtgacttggaggctggcaagaacctacccatgatctacggagaccccccgccggaggtcatcggcatccccctggaggacctggatccctactacagcaataagaagaccttcatcgtactcaacaagggcaaggccatcttccgcttctccgccacacctgctctctacctgctgagccccttcagcgtagtcaggcgcggggccatcaaggtgctcatccatgcgctgttcagcatgttcatcatgatcaccatcttgaccaactgcgtattcatgaccatgagtgacccgcctccctggtccaagaatgtggagtacaccttcacagggatctacacctttgagtccctcatcaagatactggcccgaggcttctgtgtcgacgacttcacattcctccgggacccctggaactggctggacttcagtgtcatcatgatggcgtacctgacagagtttgtggacttgggcaacatctcagccctgaggaccttccgggtgctgcgggccctcaaaaccatcacggtcatcccagggctgaagacgatcgtgggggccctgatccagtcggtgaaaaagctgtcggatgtgatgatcctcactgtcttctgcctgagcgtctttgcgctggtaggactgcagctcttcatgggaaacctgaggcagaagtgtgtgcgctggcccccgccgttcaacgacaccaacaccacgtggtacagcaatgacacgtggtacggcaatgacacatggtatggcaatgagatgtggtacggcaatgactcatggtatgccaacgacacgtggaacagccatgcaagctgggccaccaacgatacctttgattgggacgcctacatcagtgatgaagggaacttctacttcctggagggctccaacgatgccctgctctgtgggaacagcagtgatgctgggcactgccctgagggttatgagtgcatcaagaccgggcggaaccccaactatggctacaccagctatgacaccttcagctgggccttcttggctctcttccgcctcatgacacaggactattgggagaacctcttccagctgacccttcgagcagctggcaagacctacatgatcttcttcgtggtcatcatcttcctgggctctttctacctcatcaatctgatcctggccgtggtggccatggcatatgccgagcagaatgaggccaccctggccgaggataaggagaaagaggaggagtttcagcagatgcttgagaagttcaaaaagcaccaggaggagctggagaaggccaaggccgcccaagctctggaaggtggggaggcagatggggacccagcccatggcaaagactgcaatggcagcctggacacatcgcaaggggagaagggagccccgaggcagagcagcagcggagacagcggcatctccgacgccatggaagaactggaagaggcccaccaaaagtgcccaccatggtggtacaagtgcgcccacaaagtgctcatatggaactgctgcgccccgtggctgaagttcaagaacatcatccacctgatcgtcatggacccgttcgtggacctgggcatcaccatctgcatcgtgctcaacaccctcttcatggccatggaacattaccccatgacggagcactttgacaacgtgctcactgtgggcaacctggtcttcacaggcatcttcacagcagagatggttctgaagctgattgccatggacccctacgagtatttccagcagggttggaatatcttcgacagcatcatcgtcaccctcagcctggtagagctaggcctggccaacgtacagggactgtctgtgctacgctccttccgtctgctgcgggtcttcaagctggccaagtcgtggccaacgctgaacatgctcatcaagatcattggcaattcagtgggggcgctgggtaacctgacgctggtgctggctatcatcgtgttcatcttcgccgtggtgggcatgcagctgtttggcaagagctacaaggagtgcgtgtgcaagattgccttggactgcaacctgccgcgctggcacatgcatgatttcttccactccttcctcatcgtcttccgcatcctgtgcggggagtggatcgagaccatgtgggactgcatggaggtggccggccaagccatgtgcctcaccgtcttcctcatggtcatggtcatcggcaatcttgtggtcctgaacctgttcctggctctgctgctgagctccttcagcgccgacagtctggcagcctcggatgaggatggcgagatgaacaacctgcagattgccatcgggcgcatcaagttgggcatcggctttgccaaggccttcctcctggggctgctgcatggcaagatcctgagccccaaggacatcatgctcagcctcggggaggctgacggggccggggaggctggagaggcgggggagactgcccccgaggatgagaagaaggagccgcccgaggaggacctgaagaaggacaatcacatcctgaaccacatgggcctggctgacggccccccatccagcctcgagctggaccaccttaacttcatcaacaacccctacctgaccatacaggtgcccatcgcctccgaggagtccgacctggagatgcccaccgaggaggaaaccgacactttctcagagcctgaggatagcaagaagccgccgcagcctctctatgatgggaactcgtccgtctgcagcacagctgactacaagccccccgaggaggaccctgaggagcaggcagaggagaaccccgagggggagcagcctgaggagtgcttcactgaggcctgcgtgcagcgctggccctgcctctacgtggacatctcccagggccgtgggaagaagtggtggactctgcgcagggcctgcttcaagattgtcgagcacaactggttcgagaccttcattgtcttcatgatcctgctcagcagtggggctctggccttcgaggacatctacattgagcagcggcgagtcattcgcaccatcctagaatatgccgacaaggtcttcacctacatcttcatcatggagatgctgctcaaatgggtggcctacggctttaaggtgtacttcaccaacgcctggtgctggctcgacttcctcatcgtggatgtctccatcatcagcttggtggccaactggctgggctactcggagctgggacccatcaaatccctgcggacactgcgggccctgcgtcccctgagggcactgtcccgattcgagggcatgagggtggtggtgaacgccctcctaggcgccatcccctccatcatgaatgtgctgcttgtctgcctcatcttctggctgatcttcagcatcatgggtgtcaacctgtttgccggcaagttctactactgcatcaacaccaccacctctgagaggttcgacatctccgaggtcaacaacaagtctgagtgcgagagcctcatgcacacaggccaggtccgctggctcaatgtcaaggtcaactacgacaacgtgggtctgggctacctctccctcctgcaggtggccaccttcaagggttggatggacatcatgtatgcagccgtggactcccgggagaaggaggagcagccgcagtacgaggtgaacctctacatgtacctctactttgtcatcttcatcatctttggctccttcttcaccctcaacctcttcattggcgtcatcattgacaacttcaaccagcagaagaagaagttaggggggaaagacatctttatgacggaggaacagaagaaatactataacgccatgaagaagcttggctccaagaagcctcagaagccaattccccggccccagaacaagatccagggcatggtgtatgacctcgtgacgaagcaggccttcgacatcaccatcatgatcctcatctgcctcaacatggtcaccatgatggtggagacagacaaccagagccagctcaaggtggacatcctgtacaacatcaacatgatcttcatcatcatcttcacaggggagtgcgtgctcaagatgctcgccctgcgccagtactacttcaccgttggctggaacatctttgacttcgtggtcgtcatcctgtccattgtgggccttgccctctctgacctgatccagaagtacttcgtgtcacccacgctgttccgtgtgatccgcctggcgcggattgggcgtgtcctgcggctgatccgcggggccaagggcatccggacgctgctgttcgccctcatgatgtcgctgcctgccctcttcaacatcggcctcctcctcttcctggtcatgttcatctactccatcttcggcatgtccaactttgcctacgtcaagaaggagtcgggcatcgatgatatgttcaacttcgagaccttcggcaacagcatcatctgcctgttcgagatcaccacgtcggccggctgggacgggctcctcaaccccatcctcaacagcgggcccccagactgtgaccccaacctggagaacccgggcaccagtgtcaagggtgactgcggcaacccctccatcggcatctgcttcttctgcagctatatcatcatctccttcctcatcgtggtcaacatgtacatcgccatcatcctggagaacttcaatgtggccacagaggagagcagcgagccccttggtgaagatgactttgagatgttctacgagacatgggagaagttcgaccccgacgccacccagttcatcgcctacagccgcctctcagacttcgtggacaccctgcaggaaccgctgaggattgccaagcccaacaagatcaagctcatcacactggacttgcccatggtgccaggggacaagatccactgcctggacatcctctttgccctgaccaaagaggtcctgggtgactctggggaaatggacgccctcaagcagaccatggaggagaagttcatggcagccaacccctccaaggtgtcctacgagcccatcaccaccaccctcaagaggaagcacgaggaggtgtgcgccatcaagatccagagggcctaccgccggcacctgctacagcgctccatgaagcaggcatcctacatgtaccgccacagccacgacggcagcggggatgacgcccctgagaaggaggggctgcttgccaacaccatgagcaagatgtatggccacgagaatgggaacagcagctcgccaagcccggaggagaagggcgaggcaggggacgccggacccactatggggctgatgcccatcagcccctcagacactgcctggcctcccgcccctcccccagggcagactgtgcgcccaggtgtcaaggagtctcttgtctag

SEQ ID NO: 110 - protein, 1836 aa, Homo Sapiens
MARPSLCTLVPLGPECLRPFTRESLAAIEQRAVEEEARLQRNKQMEIEEPERKPRSDLEAGKNLPMIYGDPPPEVIGIPLEDLDPYYSNKKTFIVLNKGKAIFRFSATPALYLLSPFSVVRRGAIKVLIHALFSMFIMITILTNCVFMTMSDPPPWSKNVEYTFTGIYTFESLIKILARGFCVDDFTFLRDPWNWLDFSVIMMAYLTEFVDLGNISALRTFRVLRALKTITVIPGLKTIVGALIQSVKKLSDVMILTVFCLSVFALVGLQLFMGNLRQKCVRWPPPFNDTNTTWYSNDTWYGNDTWYGNEMWYGNDSWYANDTWNSHASWATNDTFDWDAYISDEGNFYFLEGSNDALLCGNSSDAGHCPEGYECIKTGRNPNYGYTSYDTFSWAFLALFRLMTQDYWENLFQLTLRAAGKTYMIFFVVIIFLGSFYLINLILAVVAMAYAEQNEATLAEDKEKEEEFQQMLEKFKKHQEELEKAKAAQALEGGEADGDPAHGKDCNGSLDTSQGEKGAPRQSSSGDSGISDAMEELEEAHQKCPPWWYKCAHKVLIWNCCAPWLKFKNIIHLIVMDPFVDLGITICIVLNTLFMAMEHYPMTEHFDNVLTVGNLVFTGIFTAEMVLKLIAMDPYEYFQQGWNIFDSIIVTLSLVELGLANVQGLSVLRSFRLLRVFKLAKSWPTLNMLIKIIGNSVGALGNLTLVLAIIVFIFAVVGMQLFGKSYKECVCKIALDCNLPRWHMHDFFHSFLIVFRILCGEWIETMWDCMEVAGQAMCLTVFLMVMVIGNLVVLNLFLALLLSSFSADSLAASDEDGEMNNLQIAIGRIKLGIGFAKAFLLGLLHGKILSPKDIMLSLGEADGAGEAGEAGETAPEDEKKEPPEEDLKKDNHILNHMGLADGPPSSLELDHLNFINNPYLTIQVPIASEESDLEMPTEEETDTFSEPEDSKKPPQPLYDGNSSVCSTADYKPPEEDPEEQAEENPEGEQPEECFTEACVQRWPCLYVDISQGRGKKWWTLRRACFKIVEHNWFETFIVFMILLSSGALAFEDIYIEQRRVIRTILEYADKVFTYIFIMEMLLKWVAYGFKVYFTNAWCWLDFLIVDVSIISLVANWLGYSELGPIKSLRTLRALRPLRALSRFEGMRVVVNALLGAIPSIMNVLLVCLIFWLIFSIMGVNLFAGKFYYCINTTTSERFDISEVNNKSECESLMHTGQVRWLNVKVNYDNVGLGYLSLLQVATFKGWMDIMYAAVDSREKEEQPQYEVNLYMYLYFVIFIIFGSFFTLNLFIGVIIDNFNQQKKKLGGKDIFMTEEQKKYYNAMKKLGSKKPQKPIPRPQNKIQGMVYDLVTKQAFDITIMILICLNMVTMMVETDNQSQLKVDILYNINMIFIIIFTGECVLKMLALRQYYFTVGWNIFDFVVVILSIVGLALSDLIQKYFVSPTLFRVIRLARIGRVLRLIRGAKGIRTLLFALMMSLPALFNIGLLLFLVMFIYSIFGMSNFAYVKKESGIDDMFNFETFGNSIICLFEITTSAGWDGLLNPILNSGPPDCDPNLENPGTSVKGDCGNPSIGICFFCSYIIISFLIVVNMYIAIILENFNVATEESSEPLGEDDFEMFYETWEKFDPDATQFIAYSRLSDFVDTLQEPLRIAKPNKIKLITLDLPMVPGDKIHCLDILFALTKEVLGDSGEMDALKQTMEEKFMAANPSKVSYEPITTTLKRKHEEVCAIKIQRAYRRHLLQRSMKQASYMYRHSHDGSGDDAPEKEGLLANTMSKMYGHENGNSSSPSPEEKGEAGDAGPTMGLMPISPSDTAWPPAPPPGQTVRPGVKESLV

SEQ ID NO: 111 - protein, 8 aa, Homo Sapiens
FMTMSDPP

SEQ ID NO: 112 - protein, 9 aa, Homo Sapiens
LTEFVDLGN

SEQ ID NO: 113 - protein, 149 aa, Homo Sapiens
MGNLRQKCVRWPPPFNDTNTTWYSNDTWYGNDTWYGNEMWYGNDSWYANDTWNSHASWATNDTFDWDAYISDEGNFYFLEGSNDALLCGNSSDAGHCPEGYECIKTGRNPNYGYTSYDTFSWAFLALFRLMTQDYWENLFQLTLRAAGK

SEQ ID NO: 114 - protein, 14 aa, Homo Sapiens
VGMFLADLIETYFV

SEQ ID NO: 115 - protein, 13 aa, Homo Sapiens
MAMEHYPMTEHFD

SEQ ID NO: 116 - protein, 10 aa, Homo Sapiens
VELGLANVQG

SEQ ID NO: 117 - protein, 54 aa, Homo Sapiens
GKSYKECVCKIALDCNLPRWHMHDFFHSFLIVFRILCGEWIETMWDCMEVAGQA

SEQ ID NO: 118 - protein, 9 aa, Homo Sapiens
TVLSDIIQK

SEQ ID NO: 119 - protein, 15 aa, Homo Sapiens
LAFEDIYIEQRRVIR

SEQ ID NO: 120 - protein, 17 aa, Homo Sapiens
ISLVANWLGYSELGPIK

SEQ ID NO: 121 - protein, 87 aa, Homo Sapiens
AGKFYYCINTTTSERFDISEVNNKSECESLMHTGQVRWLNVKVNYDNVGLGYLSLLQVATFKGWMDIMYAAVDSREKEEQPQYEVNL

SEQ ID NO: 122 - protein, 5 aa, Homo Sapiens
QKYFV

SEQ ID NO: 123 - protein, 14 aa, Homo Sapiens
MMVETDNQSQLKVD

SEQ ID NO: 124 - protein, 20 aa, Homo Sapiens
VGLALSDLIQKYFVSPTLFR

SEQ ID NO: 125 - protein, 68 aa, Homo Sapiens
AYVKKESGIDDMFNFETFGNSIICLFEITTSAGWDGLLNPILNSGPPDCDPNLENPGTSVKGDCGNPS

SEQ ID NO: 126 - protein, 6 aa, Homo Sapiens
SDLIQK

SEQ ID NO: 127 - DNA, 6051 bp, Homo Sapiens
atggcaaacttcctattacctcggggcaccagcagcttccgcaggttcacacgggagtccctggcagccatcgagaagcgcatggcagagaagcaagcccgcggctcaaccaccttgcaggagagccgagaggggctgcccgaggaggaggctccccggccccagctggacctgcaggcctccaaaaagctgccagatctctatggcaatccaccccaagagctcatcggagagcccctggaggacctggaccccttctatagcacccaaaagactttcatcgtactgaataaaggcaagaccatcttccggttcagtgccaccaacgccttgtatgtcctcagtcccttccaccccatccggagagcggctgtgaagattctggttcactcgctcttcaacatgctcatcatgtgcaccatcctcaccaactgcgtgttcatggcccagcacgaccctccaccctggaccaagtatgtcgagtacaccttcaccgccatttacacctttgagtctctggtcaagattctggctcgaggcttctgcctgcacgcgttcactttccttcgggacccatggaactggctggactttagtgtgattatcatggcatacacaactgaatttgtggacctgggcaatgtctcagccttacgcaccttccgagtcctccgggccctgaaaactatatcagtcatttcagggctgaagaccatcgtgggggccctgatccagtctgtgaagaagctggctgatgtgatggtcctcacagtcttctgcctcagcgtctttgccctcatcggcctgcagctcttcatgggcaacctaaggcacaagtgcgtgcgcaacttcacagcgctcaacggcaccaacggctccgtggaggccgacggcttggtctgggaatccctggacctttacctcagtgatccagaaaattacctgctcaagaacggcacctctgatgtgttactgtgtgggaacagctctgacgctgggacatgtccggagggctaccggtgcctaaaggcaggcgagaaccccgaccacggctacaccagcttcgattcctttgcctgggcctttcttgcactcttccgcctgatgacgcaggactgctgggagcgcctctatcagcagaccctcaggtccgcagggaagatctacatgatcttcttcatgcttgtcatcttcctggggtccttctacctggtgaacctgatcctggccgtggtcgcaatggcctatgaggagcaaaaccaagccaccatcgctgagaccgaggagaaggaaaagcgcttccaggaggccatggaaatgctcaagaaagaacacgaggccctcaccatcaggggtgtggataccgtgtcccgtagctccttggagatgtcccctttggccccagtaaacagccatgagagaagaagcaagaggagaaaacggatgtcttcaggaactgaggagtgtggggaggacaggctccccaagtctgactcagaagatggtcccagagcaatgaatcatctcagcctcacccgtggcctcagcaggacttctatgaagccacgttccagccgcgggagcattttcacctttcgcaggcgagacctgggttctgaagcagattttgcagatgatgaaaacagcacagcgggggagagcgagagccaccacacatcactgctggtgccctggcccctgcgccggaccagtgcccagggacagcccagtcccggaacctcggctcctggccacgccctccatggcaaaaagaacagcactgtggactgcaatggggtggtctcattactgggggcaggcgacccagaggccacatccccaggaagccacctcctccgccctgtgatgctagagcacccgccagacacgaccacgccatcggaggagccaggcgggccccagatgctgacctcccaggctccgtgtgtagatggcttcgaggagccaggagcacggcagcgggccctcagcgcagtcagcgtcctcaccagcgcactggaagagttagaggagtctcgccacaagtgtccaccatgctggaaccgtctcgcccagcgctacctgatctgggagtgctgcccgctgtggatgtccatcaagcagggagtgaagttggtggtcatggacccgtttactgacctcaccatcactatgtgcatcgtactcaacacactcttcatggcgctggagcactacaacatgacaagtgaattcgaggagatgctgcaggtcggaaacctggtcttcacagggattttcacagcagagatgaccttcaagatcattgccctcgacccctactactacttccaacagggctggaacatcttcgacagcatcatcgtcatccttagcctcatggagctgggcctgtcccgcatgagcaacttgtcggtgctgcgctccttccgcctgctgcgggtcttcaagctggccaaatcatggcccaccctgaacacactcatcaagatcatcgggaactcagtgggggcactggggaacctgacactggtgctagccatcatcgtgttcatctttgctgtggtgggcatgcagctctttggcaagaactactcggagctgagggacagcgactcaggcctgctgcctcgctggcacatgatggacttctttcatgccttcctcatcatcttccgcatcctctgtggagagtggatcgagaccatgtgggactgcatggaggtgtcggggcagtcattatgcctgctggtcttcttgcttgttatggtcattggcaaccttgtggtcctgaatctcttcctggccttgctgctcagctccttcagtgcagacaacctcacagcccctgatgaggacagagagatgaacaacctccagctggccctggcccgcatccagaggggcctgcgctttgtcaagcggaccacctgggatttctgctgtggtctcctgcggcagcggcctcagaagcccgcagcccttgccgcccagggccagctgcccagctgcattgccaccccctactccccgccacccccagagacggagaaggtgcctcccacccgcaaggaaacacggtttgaggaaggcgagcaaccaggccagggcacccccggggatccagagcccgtgtgtgtgcccatcgctgtggccgagtcagacacagatgaccaagaagaagatgaggagaacagcctgggcacggaggaggagtccagcaagcagcaggaatcccagcctgtgtccggtggcccagaggcccctccggattccaggacctggagccaggtgtcagcgactgcctcctctgaggccgaggccagtgcatctcaggccgactggcggcagcagtggaaagcggaaccccaggccccagggtgcggtgagaccccagaggacagttgctccgagggcagcacagcagacatgaccaacaccgctgagctcctggagcagatccctgacctcggccaggatgtcaaggacccagaggactgcttcactgaaggctgtgtccggcgctgtccctgctgtgcggtggacaccacacaggccccagggaaggtctggtggcggttgcgcaagacctgctaccacatcgtggagcacagctggttcgagacattcatcatcttcatgatcctactcagcagtggagcgctggccttcgaggacatctacctagaggagcggaagaccatcaaggttctgcttgagtatgccgacaagatgttcacatatgtcttcgtgctggagatgctgctcaagtgggtggcctacggcttcaagaagtacttcaccaatgcctggtgctggctcgacttcctcatcgtagacgtctctctggtcagcctggtggccaacaccctgggctttgccgagatgggccccatcaagtcactgcggacgctgcgtgcactccgtcctctgagagctctgtcacgatttgagggcatgagggtggtggtcaatgccctggtgggcgccatcccgtccatcatgaacgtcctcctcgtctgcctcatcttctggctcatcttcagcatcatgggcgtgaacctctttgcggggaagtttgggaggtgcatcaaccagacagagggagacttgcctttgaactacaccatcgtgaacaacaagagccagtgtgagtccttgaacttgaccggagaattgtactggaccaaggtgaaagtcaactttgacaacgtgggggccgggtacctggcccttctgcaggtggcaacatttaaaggctggatggacattatgtatgcagctgtggactccagggggtatgaagagcagcctcagtgggaatacaacctctacatgtacatctattttgtcattttcatcatctttgggtctttcttcaccctgaacctctttattggtgtcatcattgacaacttcaaccaacagaagaaaaagttagggggccaggacatcttcatgacagaggagcagaagaagtactacaatgccatgaagaagctgggctccaagaagccccagaagcccatcccacggcccctgaacaagtaccagggcttcatattcgacattgtgaccaagcaggcctttgacgtcaccatcatgtttctgatctgcttgaatatggtgaccatgatggtggagacagatgaccaaagtcctgagaaaatcaacatcttggccaagatcaacctgctctttgtggccatcttcacaggcgagtgtattgtcaagctggctgccctgcgccactactacttcaccaacagctggaatatcttcgacttcgtggttgtcatcctctccatcgtgggcactgtgctctcggacatcatccagaagtacttcttctccccgacgctcttccgagtcatccgcctggcccgaataggccgcatcctcagactgatccgaggggccaaggggatccgcacgctgctctttgccctcatgatgtccctgcctgccctcttcaacatcgggctgctgctcttcctcgtcatgttcatctactccatctttggcatggccaacttcgcttatgtcaagtgggaggctggcatcgacgacatgttcaacttccagaccttcgccaacagcatgctgtgcctcttccagatcaccacgtcggccggctgggatggcctcctcagccccatcctcaacactgggccgccctactgcgaccccactctgcccaacagcaatggctctcggggggactgcgggagcccagccgtgggcatcctcttcttcaccacctacatcatcatctccttcctcatcgtggtcaacatgtacattgccatcatcctggagaacttcagcgtggccacggaggagagcaccgagcccctgagtgaggacgacttcgatatgttctatgagatctgggagaaatttgacccagaggccactcagtttattgagtattcggtcctgtctgactttgccgatgccctgtctgagccactccgtatcgccaagcccaaccagataagcctcatcaacatggacctgcccatggtgagtggggaccgcatccattgcatggacattctctttgccttcaccaaaagggtcctgggggagtctggggagatggacgccctgaagatccagatggaggagaagttcatggcagccaacccatccaagatctcctacgagcccatcaccaccacactccggcgcaagcacgaagaggtgtcggccatggttatccagagagccttccgcaggcacctgctgcaacgctctttgaagcatgcctccttcctcttccgtcagcaggcgggcagcggcctctccgaagaggatgcccctgagcgagagggcctcatcgcctacgtgatgagtgagaacttctcccgaccccttggcccaccctccagctcctccatctcctccacttccttcccaccctcctatgacagtgtcactagagccaccagcgataacctccaggtgcgggggtctgactacagccacagtgaagatctcgccgacttccccccttctccggacagggaccgtgagtccatcgtgtga

SEQ ID NO: 128 - protein, 2016 aa, Homo Sapiens [sequence omitted]

SEQ ID NO: 129 - protein, 8 aa, Homo Sapiens
FMAQHDPP

SEQ ID NO: 130 - protein, 9 aa, Homo Sapiens
TTEFVDLGN

SEQ ID NO: 131 - protein, 115 aa, Homo Sapiens
MGNLRHKCVRNFTALNGTNGSVEADGLVWESLDLYLSDPENYLLKNGTSDVLLCGNSSDAGTCPEGYRCLKAGENPDHGYTSFDSFAWAFLALFRLMTQDCWERLYQQTLRSAGK

SEQ ID NO: 132 - protein, 12 aa, Homo Sapiens
MFLAEMIEKYFV

SEQ ID NO: 133 - protein, 13 aa, Homo Sapiens
MALEHYNMTSEFE

SEQ ID NO: 134 - protein, 10 aa, Homo Sapiens
MELGLSRMSN

SEQ ID NO: 135 - protein, 52 aa, Homo Sapiens
GKNYSELRDSDSGLLPRWHMMDFFHAFLIIFRILCGEWIETMWDCMEVSGQS

SEQ ID NO: 136 - protein, 6 aa, Homo Sapiens
AEMIEK

SEQ ID NO: 137 - protein, 15 aa, Homo Sapiens
LAFEDIYLEERKTIK

SEQ ID NO: 138 - protein, 17 aa, Homo Sapiens
VSLVANTLGFAEMGPIK

SEQ ID NO: 139 - protein, 88 aa, Homo Sapiens
AGKFGRCINQTEGDLPLNYTIVNNKSQCESLNLTGELYWTKVKVNFDNVGAGYLALLQVATFKGWMDIMYAAVDSRGYEEQPQWEYNL

SEQ ID NO: 140 - protein, 9 aa, Homo Sapiens
MFLAEMIEK

SEQ ID NO: 141 - protein, 14 aa, Homo Sapiens
MMVETDDQSPEKIN

SEQ ID NO: 142 - protein, 20 aa, Homo Sapiens
VGTVLSDIIQKYFFSPTLFR

SEQ ID NO: 143 - protein, 67 aa, Homo Sapiens
AYVKWEAGIDDMFNFQTFANSMLCLFQITTSAGWDGLLSPILNTGPPYCDPTLPNSNGSRGDCGSPA

SEQ ID NO: 144 - protein, 12 aa, Homo Sapiens
MFLAELIEKYFV

SEQ ID NO: 145 - DNA, 5943 bp, Homo Sapiens
atggcagcgcggctgcttgcaccaccaggccctgatagtttcaagcctttcacccctgagtcactggcaaacattgagaggcgcattgctgagagcaagctcaagaaaccaccaaaggccgatggcagtcatcgggaggacgatgaggacagcaagcccaagccaaacagcgacctggaagcagggaagagtttgcctttcatctacggggacatcccccaaggcctggttgcagttcccctggaggactttgacccatactatttgacgcagaaaacctttgtagtattaaacagagggaaaactctcttcagatttagtgccacgcctgccttgtacattttaagtccttttaacctgataagaagaatagctattaaaattttgatacattcagtatttagcatgatcattatgtgcactattttgaccaactgtgtattcatgacttttagtaaccctcctgactggtcgaagaatgtggagtacacgttcacagggatttatacatttgaatcactagtgaaaatcattgcaagaggtttctgcatagatggctttacctttttacgggacccatggaactggttagatttcagtgtcatcatgatggcgtatataacagagtttgtaaacctaggcaatgtttcagctctacgcactttcagggtactgagggctttgaaaactatttcggtaatcccaggcctgaagacaattgtgggtgccctgattcagtctgtgaagaaactgtcagatgtgatgatcctgacagtgttctgcctgagtgtttttgccttgatcggactgcagctgttcatggggaaccttcgaaacaagtgtgttgtgtggcccataaacttcaacgagagctatcttgaaaatggcaccaaaggctttgattgggaagagtatatcaacaataaaacaaatttctacacagttcctggcatgctggaacctttactctgtgggaacagttctgatgctgggcaatgcccagagggataccagtgtatgaaagcaggaaggaaccccaactatggttacacaagttttgacacttttagctgggccttcttggcattatttcgccttatgacccaggactattgggaaaacttgtatcaattgactttacgagcagccgggaaaacatacatgatcttcttcgtcttggtcatctttgtgggttctttctatctggtgaacttgatcttggctgtggtggccatggcttatgaagaacagaatcaggcaacactggaggaggcagaacaaaaagaggctgaatttaaagcaatgttggagcaacttaagaagcaacaggaagaggcacaggctgctgcgatggccacttcagcaggaactgtctcagaagatgccatagaggaagaaggtgaagaaggagggggctcccctcggagctcttctgaaatctctaaactcagctcaaagagtgcaaaggaaagacgtaacaggagaaagaagaggaagcaaaaggaactctctgaaggagaggagaaaggggatcccgagaaggtgtttaagtcagagtcagaagatggcatgagaaggaaggcctttcggctgccagacaacagaatagggaggaaattttccatcatgaatcagtcactgctcagcatcccaggctcgcccttcctctcccgccacaacagcaagagcagcatcttcagtttcaggggacctgggcggttccgagacccgggctccgagaatgagttcgcggatgacgagcacagcacggtggaggagagcgagggccgccgggactccctcttcatccccatccgggcccgcgagcgccggagcagctacagcggctacagcggctacagccagggcagccgctcctcgcgcatcttccccagcctgcggcgcagcgtgaagcgcaacagcacggtggactgcaacggcgtggtgtccctcatcggcggccccggctcccacatcggcgggcgtctcctgccagaggctacaactgaggtggaaattaagaagaaaggccctggatctcttttagtttccatggaccaattagcctcctacgggcggaaggacagaatcaacagtataatgagtgttgttacaaatacactagtagaagaactggaagagtctcagagaaagtgcccgccatgctggtataaatttgccaacactttcctcatctgggagtgccacccctactggataaaactgaaagagattgtgaacttgatagttatggacccttttgtggatttagccatcaccatctgcatcgtcctgaatacactgtttatggcaatggagcaccatcctatgacaccacaatttgaacatgtcttggctgtaggaaatctggttttcactggaattttcacagcggaaatgttcctgaagctcatagccatggatccctactattatttccaagaaggttggaacatttttgacggatttattgtctccctcagtttaatggaactgagtctagcagacgtggaggggctttcagtgctgcgatctttccgattgctccgagtcttcaaattggccaaatcctggcccaccctgaacatgctaatcaagattattggaaattcagtgggtgccctgggcaacctgacactggtgctggccattattgtcttcatctttgccgtggtggggatgcaactctttggaaaaagctacaaagagtgtgtctgcaagatcaaccaggactgtgaactccctcgctggcatatgcatgactttttccattccttcctcattgtctttcgagtgttgtgcggggagtggattgagaccatgtgggactgcatggaagtggcaggccaggccatgtgcctcattgtctttatgatggtcatggtgattggcaacttggtggtgctgaacctgtttctggccttgctcctgagctccttcagtgcagacaacctggctgccacagatgacgatggggaaatgaacaacctccagatctcagtgatccgtatcaagaagggtgtggcctggaccaaactaaaggtgcacgccttcatgcaggcccactttaagcagcgtgaggctgatgaggtgaagcctctggatgagttgtatgaaaagaaggccaactgtatcgccaatcacaccggtgcagacatccaccggaatggtgacttccagaagaatggcaatggcacaaccagcggcattggcagcagcgtggagaagtacatcattgatgaggaccacatgtccttcatcaacaaccccaacttgactgtacgggtacccattgctgtgggcgagtctgactttgagaacctcaacacagaggatgttagcagcgagtcggatcctgaaggcagcaaagataaactagatgacaccagctcctctgaaggaagcaccattgatatcaaaccagaagtagaagaggtccctgtggaacagcctgaggaatacttggatccagatgcctgcttcacagaaggttgtgtccagcggttcaagtgctgccaggtcaacatcgaggaagggctaggcaagtcttggtggatcctgcggaaaacctgcttcctcatcgtggagcacaactggtttgagaccttcatcatcttcatgattctgctgagcagtggcgccctggccttcgaggacatctacattgagcagagaaagaccatccgcaccatcctggaatatgctgacaaagtcttcacctatatcttcatcctggagatgttgctcaagtggacagcctatggcttcgtcaagttcttcaccaatgcctggtgttggctggacttcctcattgtggctgtctctttagtcagccttatagctaatgccctgggctactcggaactaggtgccataaagtcccttaggaccctaagagctttgagacccttaagagccttatcacgatttgaagggatgagggtggtggtgaatgccttggtgggcgccatcccctccatcatgaatgtgctgctggtgtgtctcatcttctggctgattttcagcatcatgggagttaacttgtttgcgggaaagtaccactactgctttaatgagacttctgaaatccgatttgaaattgaagatgtcaacaataaaactgaatgtgaaaagcttatggaggggaacaatacagagatcagatggaagaacgtgaagatcaactttgacaatgttggggcaggatacctggcccttcttcaagtagcaaccttcaaaggctggatggacatcatgtatgcagctgtagattcccggaagcctgatgagcagcctaagtatgaggacaatatctacatgtacatctattttgtcatcttcatcatcttcggctccttcttcaccctgaacctgttcattggtgtcatcattgataacttcaatcaacaaaagaaaaagttcggaggtcaggacatcttcatgaccgaagaacagaagaagtactacaatgccatgaaaaagctgggctcaaagaagccacagaaacctattccccgccccttgaacaaaatccaaggaatcgtctttgattttgtcactcagcaagcctttgacattgttatcatgatgctcatctgccttaacatggtgacaatgatggtggagacagacactcaaagcaagcagatggagaacatcctctactggattaacctggtgtttgttatcttcttcacctgtgagtgtgtgctcaaaatgtttgcgttgaggcactactacttcaccattggctggaacatcttcgacttcgtggtagtcatcctctccattgtgggaatgttcctggcagatataattgagaaatactttgtttccccaaccctattccgagtcatccgattggcccgtattgggcgcatcttgcgtctgatcaaaggcgccaaagggattcgtaccctgctctttgccttaatgatgtccttgcctgccctgttcaacatcggccttctgctcttcctggtcatgttcatcttctccatttttgggatgtccaattttgcatatgtgaagcacgaggctggtatcgatgacatgttcaactttgagacatttggcaacagcatgatctgcctgtttcaaatcacaacctcagctggttgggatggcctgctgctgcccatcctaaaccgcccccctgactgcagcctagataaggaacacccagggagtggctttaagggagattgtgggaacccctcagtgggcatcttcttctttgtaagctacatcatcatctctttcctaattgtcgtgaacatgtacattgccatcatcctggagaacttcagtgtagccacagaggaaagtgcagaccctctgagtgaggatgactttgagaccttctatgagatctgggagaagttcgaccccgatgccacccagttcattgagtactgtaagctggcagactttgcagatgccttggagcatcctctccgagtgcccaagcccaataccattgagctcatcgctatggatctgccaatggtgagcggggatcgcatccactgcttggacatcctttttgccttcaccaagcgggtcctgggagatagcggggagttggacatcctgcggcagcagatggaagagcggttcgtggcatccaatccttccaaagtgtcttacgagccaatcacaaccacactgcgtcgcaagcaggaggaggtatctgcagtggtcctgcagcgtgcctaccggggacatttggcaaggcggggcttcatctgcaaaaagacaacttctaataagctggagaatggaggcacacaccgggagaaaaaagagagcaccccatctacagcctccctcccgtcctatgacagtgtaactaaacctgaaaaggagaaacagcagcgggcagaggaaggaagaagggaaagagccaaaagacaaaaagaggtcagagaatccaagtgttag

SEQ ID NO: 146 - protein, 1980 aa, Homo Sapiens
MAARLLAPPGPDSFKPFTPESLANIERRIAESKLKKPPKADGSHREDDEDSKPKPNSDLEAGKSLPFIYGDIPQGLVAVPLEDFDPYYLTQKTFVVLNRGKTLFRFSATPALYILSPFNLIRRIAIKILIHSVFSMIIMCTILTNCVFMTFSNPPDWSKNVEYTFTGIYTFESLVKIIARGFCIDGFTFLRDPWNWLDFSVIMMAYITEFVNLGNVSALRTFRVLRALKTISVIPGLKTIVGALIQSVKKLSDVMILTVFCLSVFALIGLQLFMGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGKTYMIFFVLVIFVGSFYLVNLILAVVAMAYEEQNQATLEEAEQKEAEFKAMLEQLKKQQEEAQAAAMATSAGTVSEDAIEEEGEEGGGSPRSSSEISKLSSKSAKERRNRRKKRKQKELSEGEEKGDPEKVFKSESEDGMRRKAFRLPDNRIGRKFSIMNQSLLSIPGSPFLSRHNSKSSIFSFRGPGRFRDPGSENEFADDEHSTVEESEGRRDSLFIPIRARERRSSYSGYSGYSQGSRSSRIFPSLRRSVKRNSTVDCNGVVSLIGGPGSHIGGRLLPEATTEVEIKKKGPGSLLVSMDQLASYGRKDRINSIMSVVTNTLVEELEESQRKCPPCWYKFANTFLIWECHPYWIKLKEIVNLIVMDPFVDLAITICIVLNTLFMAMEHHPMTPQFEHVLAVGNLVFTGIFTAEMFLKLIAMDPYYYFQEGWNIFDGFIVSLSLMELSLADVEGLSVLRSFRLLRVFKLAKSWPTLNMLIKIIGNSVGALGNLTLVLAIIVFIFAVVGMQLFGKSYKECVCKINQDCELPRWHMHDFFHSFLIVFRVLCGEWIETMWDCMEVAGQAMCLIVFMMVMVIGNLVVLNLFLALLLSSFSADNLAATDDDGEMNNLQISVIRIKKGVAWTKLKVHAFMQAHFKQREADEVKPLDELYEKKANCIANHTGADIHRNGDFQKNGNGTTSGIGSSVEKYIIDEDHMSFINNPNLTVRVPIAVGESDFENLNTEDVSSESDPEGSKDKLDDTSSSEGSTIDIKPEVEEVPVEQPEEYLDPDACFTEGCVQRFKCCQVNIEEGLGKSWWILRKTCFLIVEHNWFETFIIFMILLSSGALAFEDIYIEQRKTIRTILEYADKVFTYIFILEMLLKWTAYGFVKFFTNAWCWLDFLIVAVSLVSLIANALGYSELGAIKSLRTLRALRPLRALSRFEGMRVVVNALVGAIPSIMNVLLVCLIFWLIFSIMGVNLFAGKYHYCFNETSEIRFEIEDVNNKTECEKLMEGNNTEIRWKNVKINFDNVGAGYLALLQVATFKGWMDIMYAAVDSRKPDEQPKYEDNIYMYIYFVIFIIFGSFFTLNLFIGVIIDNFNQQKKKFGGQDIFMTEEQKKYYNAMKKLGSKKPQKPIPRPLNKIQGIVFDFVTQQAFDIVIMMLICLNMVTMMVETDTQSKQMENILYWINLVFVIFFTCECVLKMFALRHYYFTIGWNIFDFVVVILSIVGMFLADIIEKYFVSPTLFRVIRLARIGRILRLIKGAKGIRTLLFALMMSLPALFNIGLLLFLVMFIFSIFGMSNFAYVKHEAGIDDMFNFETFGNSMICLFQITTSAGWDGLLLPILNRPPDCSLDKEHPGSGFKGDCGNPSVGIFFFVSYIIISFLIVVNMYIAIILENFSVATEESADPLSEDDFETFYEIWEKFDPDATQFIEYCKLADFADALEHPLRVPKPNTIELIAMDLPMVSGDRIHCLDILFAFTKRVLGDSGELDILRQQMEERFVASNPSKVSYEPITTTLRRKQEEVSAVVLQRAYRGHLARRGFICKKTTSNKLENGGTHREKKESTPSTASLPSYDSVTKPEKEKQQRAEEGRRERAKRQKEVRESKC

SEQ ID NO: 147 - protein, 8 aa, Homo Sapiens
FMTFSNPP

SEQ ID NO: 148 - protein, 9 aa, Homo Sapiens
ITEFVNLGN

SEQ ID NO: 149 - protein, 112 aa, Homo Sapiens
MGNLRNKCVVWPINFNESYLENGTKGFDWEEYINNKTNFYTVPGMLEPLLCGNSSDAGQCPEGYQCMKAGRNPNYGYTSFDTFSWAFLALFRLMTQDYWENLYQLTLRAAGK

SEQ ID NO: 150 - protein, 6 aa, Homo Sapiens
AELIEK

SEQ ID NO: 151 - protein, 13 aa, Homo Sapiens
MAMEHHPMTPQFE

SEQ ID NO: 152 - protein, 10 aa, Homo Sapiens
MELSLADVEG

SEQ ID NO: 153 - protein, 54 aa, Homo Sapiens
GKSYKECVCKINQDCELPRWHMHDFFHSFLIVFRVLCGEWIETMWDCMEVAGQA

SEQ ID NO: 154 - protein, 9 aa, Homo Sapiens
MFLAELIEK

SEQ ID NO: 155 - protein, 15 aa, Homo Sapiens
LAFEDIYIEQRKTIR

SEQ ID NO: 156 - protein, 17 aa, Homo Sapiens
VSLIANALGYSELGAIK

SEQ ID NO: 157 - protein, 89 aa, Homo Sapiens
AGKYHYCFNETSEIRFEIEDVNNKTECEKLMEGNNTEIRWKNVKINFDNVGAGYLALLQVATFKGWMDIMYAAVDSRKPDEQPKYEDNI

SEQ ID NO: 158 - protein, 14 aa, Homo Sapiens
VTLVANTLGYSDLG

SEQ ID NO: 159 - protein, 14 aa, Homo Sapiens
MMVETDTQSKQMEN

SEQ ID NO: 160 - protein, 20 aa, Homo Sapiens
VGMFLADIIEKYFVSPTLFR

SEQ ID NO: 161 - protein, 67 aa, Homo Sapiens
AYVKHEAGIDDMFNFETFGNSMICLFQITTSAGWDGLLLPILNRPPDCSLDKEHPGSGFKGDCGNPS

SEQ ID NO: 162 - protein, 14 aa, Homo Sapiens
ISLTAKILEYSEVA

SEQ ID NO: 163 - protein, 1984 aa, Rattus norvegicus
MAMLPPPGPQSFVHFTKQSLALIEQRISEEKAKEHKDEKKDDEEEGPKPSSDLEAGKQLPFIYGDIPPGMVSEPLEDLDPYYADKKTFIVLNKGKAIFRFNATPALYMLSPFSPLRRISIKILVHSLFSMLIMCTILTNCIFMTLSNPPEWTKNVEYTFTGIYTFESLIKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRKELEENETLESIMNTAESEEELKKYFYYLEGSKDALLCGFSTDSGQCPEGYICVKAGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEFTSIGRSRIMGLSESSSETSRLSSKSAKERRNRRKKKKQKMSSGEEKGDDEKLSKSGSEESIRKKSFHLGVEGHHRTREKRLSTPNQSPLSIRGSLFSARRSSRTSLFSFKGRGRDLGSETEFADDEHSIFGDNESRRGSLFVPHRPRERRSSNISQASRSPPVLPVNGKMHSAVDCNGVVSLVDGPSALMLPNGQLLPEVIIDKATSDDSGTTNQMRKKRLSSSYFLSEDMLNDPHLRQRAMSRASILTNTVEELEESRQKCPPWWYRFAHTFLIWNCSPYWIKFKKLIYFIVMDPFVDLAITICIVLNTLFMAMEHHPMTEEFKNVLAVGNLIFTGIFAAEMVLKLIAMDPYEYFQVGWNIFDSLIVTLSLIELFLADVEGLSVLRSFRLLRVFKLAKSWPTLNMLIKIIGNSVGALGNLTLVLAIIVFIFAVVGMQLFGKSYKECVCKINVDCKLPRWHMNDFFHSFLIVFRVLCGEWIETMWDCMEVAGQTMCLIVYMMVMVIGNLVVLNLFLALLLSSFSSDNLTAIEEDTDANNLQIAVARIKRGINYVKQTLREFILKSFSKKPKGSKDTKRTADPNNKKENYISNRTLAEMSKDHNFLKEKDRISGYGSSLDKSFMDENDYQSFIHNPSLTVTVPIAPGESDLEIMNTEELSSDSDSDYSKEKRNRSSSSECSTVDNPLPGEEEAEAEPVNADEPEACFTDGCVRRFPCCQVNVDSGKGKVWWTIRKTCYRIVEHSWFESFIVLMILLSSGALAFEDIYIEKKKTIKIILEYADKIFTYIFILEMLLKWVAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECVNTTDGSRFPTSQVANRSECFALMNVSGNVRWKNLKVNFDNVGLGYLSLLQVATFKGWMDIMYAAVDSVNVNEQPKYEYSLYMYIYFVIFIIFGSFFTLNLFIGVIIDNFNQQKKKLGGQDIFMTEEQKKYYNAMKKLGSKKPQKPIPRPGNKFQGCIFDLVTNQAFDITIMVLICLNMVTMMVEKEGQTEYMDYVLHWINMVFIILFTGECVLKLISLRHYYFTVGWNIFDFVVVILSIVGMFLAEMIEKYFVSPTLFRVIRLARIGRILRLIKGAKGIRTLLFALMMSLPALFNIGLLLFLVMFIYAIFGMSNFAYVKKEAGINDMFNFETFGNSMICLFQITTSAGWDGLLAPILNSAPPDCDPKKVHPGSSVEGDCGNPSVGIFYFVSYIIISFLVVVNMYIAVILENFSVATEESTEPLSEDDFEMFYEVWEKFDPDATQFIEFCKLSDFAAALDPPLLIAKPNKVQLIAMDLPMVSGDRIHCLDILFAFTKRVLGEGGEMDSLRSQMEERFMSANPSKVSYEPITTTLKRKQEEVSATIIQRAYRRYRLRQHVKNISSIYIKDGDRDDDLPNKEDTVFDNVNENSSPEKTDVTASTISPPSYDSVTKPDQEKYETDKTEKEDKEKDESRK

SEQ ID NO: 164 - protein, 1988 aa, Macaca fascicularis
MAMLPPPGPQSFVHFTKQSLALIEQRIAERKSKEPKEEKKDDDDEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMSNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCVQNSLVNNETLESIMNTLESEEDFRKYFYYLEGSKDALLCGFSTDSGQCPEGYTCMKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSDSEENIRRKSFHLGVEGHRRAHEKRLSTPSQSPLSIRGSLFSARRSSRTSLFSFKGRGRDIGSETEFADDEHSIFGDNESRRGSLFVPHRPQERRSSNISQASRSPPILPVNGKMHSAVDCNGVVSLVDGRSALMLPNGQLLPEVIIDKATSDDSGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKTFSKKPKISREIRQTEDLNTKKENYISNYTLAEMSKGHNFLKEKDKISGFGSCVDKYLMEDSDGQSFIHNPSLTVTVPIAPGESDLENMNTEELSSDSDSEYSKVRLNQSSSSECSTVDNPLPGEGEEAEAEPMNSDEPEACFTDGCVRRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVIFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSPYMTDVLYWINVVFIILFTGECVLKLISLRYYYFTIGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEYNKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 165 - protein, 1957 aa, Rattus norvegicus
MELPFASVGTTNFRRFTPESLAEIEKQIAAHRAAKKARTKHRGQEDKGEKPRPQLDLKDCNQLPKFYGELPAELVGEPLEDLDPFYSTHRTFMVLNKSRTISRFSATWALWLFSPFNLIRRTAIKVSVHSWFSIFITITILVNCVCMTRTDLPEKVEYVFTVIYTFEALIKILARGFCLNEFTYLRDPWNWLDFSVITLAYVGAAIDLRGISGLRTFRVLRALKTVSVIPGLKVIVGALIHSVRKLADVTILTVFCLSVFALVGLQLFKGNLKNKCIRNGTDPHKADNLSSEMAEYIFIKPGTTDPLLCGNGSDAGHCPGGYVCLKTPDNPDFNYTSFDSFAWAFLSLFRLMTQDSWERLYQQTLRASGKMYMVFFVLVIFLGSFYLVNLILAVVTMAYEEQSQATIAEIEAKEKKFQEALEVLQKEQEVLEALGIDTTSLQSHSGSPLASKNANERRPRVKSRVSEGSTDDNRSPQSDPYNQRRMSFLGLSSGRRRASHGSVFHFRAPSQDISFPDGITPDDGVFHGDQESRRGSILLGRGAGQTGPLPRSPLPQSPNPGRRHGEEGQLGVPTGELTAGAPEGPALHTTGQKSFLSAGYLNEPFRAQRAMSVVSIMTSVIEELEESKLKCPPCLISFAQKYLIWECCPKWRKFKMALFELVTDPFAELTITLCIVVNTVFMAMEHYPMTDAFDAMLQAGNIVFTVFFTMEMAFKIIAFDPYYYFQKKWNIFDCVIVTVSLLELSASKKGSLSVLRTLRLLRVFKLAKSWPTLNTLIKIIGNSVGALGNLTFILAIIVFIFALVGKQLLSEDYGCRKDGVSVWNGEKLRWHMCDFFHSFLVVFRILCGEWIENMWVCMEVSQKSICLILFLTVMVLGNLVVLNLFIALLLNSFSADNLTAPEDDGEVNNLQLALARIQVLGHRASRAIASYISSHCRFHWPKVETQLGMKPPLTSSEAKNHIATDAVSAAVGNLTKPALSSPKENHGDFITDPNVWVSVPIAEGESDLDELEEDMEQASQSSWQEEDPKGQQEQLPQVQKCENHQAARSPASMMSSEDLAPYLGESWKRKDSPQVPAEGVDDTSSSEGSTVDCPDPEEILRKIPELADDLDEPDDCFTEGCTRRCPCCNVNTSKSPWATGWQVRKTCYRIVEHSWFESFIIFMILLSSGALAFEDNYLEEKPRVKSVLEYTDRVFTFIFVFEMLLKWVAYGFKKYFTNAWCWLDFLIVNISLTSLIAKILEYSDVASIKALRTLRALRPLRALSRFEGMRVVVDALVGAIPSIMNVLLVCLIFWLIFSIMGVNLFAGKFSKCVDTRNNPFSNVNSTMVNNKSECHNQNSTGHFFWVNVKVNFDNVAMGYLALLQVATFKGWMDIMYAAVDSGEINSQPNWENNLYMYLYFVVFIIFGGFFTLNLFVGVIIDNFNQQKKKLGGQDIFMTEEQKKYYNAMKKLGSKKPQKPIPRPLNKYQGFVFDIVTRQAFDIIIMVLICLNMITMMVETDEQGEEKTKVLGRINQFFVAVFTGECVMKMFALRQYYFTNGWNVFDFIVVILSIGSLLFSAILKSLENYFSPTLFRVIRLARIGRILRLIRAAKGIRTLLFALMMSLPALFNIGLLLFLVMFIYSIFGMASFANVVDEAGIDDMFNFKTFGNSMLCLFQITTSAGWDGLLSPILNTGPPYCDPNLPNSNGSRGNCGSPAVGIIFFTTYIIISFLIVVNMYIAVILENFNVATEESTEPLSEDDFDMFYETWEKFDPEATQFIAFSALSDFADTLSGPLRIPKPNQNILIQMDLPLVPGDKIHCLDILFAFTKNVLGESGELDSLKTNMEEKFMATNLSKASYEPIATTLRWKQEDLSATVIQKAYRSYMLHRSLTLSNTLHVPRAEEDGVSLPGEGYITFMANSGLPDKSETASATSFPPSYDSVTRGLSDRANINPSSSMQNEDEVAAKEGNSPGPQ

SEQ ID NO: 166 - protein, 1959 aa, Macaca fascicularis
MEFPIGSLGTNNFRRFTPESLVEIEKQIAAKQAAKKAREKHREQKDQEEKTRPQLDLKACNQLPKFYGELPAELIGEPLEDLDTFYSTHRTFMVLNKGRTISRFSATRALWLFSPFNLIRRTAIKVSVHSYPLWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALINSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRASGKIYMIFFVLVIFLGSFYLVNLILAVVTMAYEEQNKATIDEIEAKEKLFQETLEKLRKEQEVLAALGIDTTSLHSHNGSPLTSKNASERRHRIKSRVSEGSTEDNKSPRSDPYNQRRMSFLGLASGKRRASHGSVFHFRSPGRDISLPEVVTDDGVFPGDHESHRGSLLLGGSAGQQGPLPRSPLPQPSNPDSRHGEDEHPLPPTSELAPGAVEVSAFDAGQKKTFLSAEYLDEPFRAQRAMSVVSIITSVLEELEESEQKCPPCLTSLAQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRIKQVLCSFFSRPCPFPRPKAEPELVVKLPLSSSKAENHIAANTAEGSSGGLQAPRGPRDEHSDFIANPTVWVSVPIAEGESDLDDLEDDGEEDAQSAQQEVIPKGQQEQLQQVERCEDHLTVRSPGTGTSSEDLAPYLGETWKDESVPQAPAEGVDDTSSSEGSTVDCPDPEEILRKIPELADDLEEPDDCFTEGCIRHCPCCKVDTTKSPWDMGWQVRKTCYRIVEHSWFESFIIFMILLSSGSLAFEDYYLDQKPTVKALLEYTDRVFTFIFVFEMLLKWVAYGFKKYFTNAWCWLDFLIVNISLTSLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTRILGKINQFFVAVFTGECVMKMFALRQYYFTNGWNVFDFIVVVLSIASLVFSAILKSLQNYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIVVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTLHVPRAEEEAASLPDEAFVAFTANENCVLPDKSETASATSFPPSYESVTRGLSDRVNMRTSSSIQNEDEATSTEVTAPGP

SEQ ID NO: 167 - protein, 1765 aa, Rattus norvegicus
MEERYYPVIFPDERNFRPFTSDSLAAIEKRIAIQKERKKSKDKAAAEPQPRPQLDLKASRKLPKLYGDIPPELVAKPLEDLDPFYKDHKTFMVLNKKRTIYRFSAKRALFILGPFNPLRSLMIRISVHSVFSMFIICTVIINCMFMANSMERSFDNDIPEYVFIGIYILEAVIKILARGFIVDEFSFLRDPWNWLDFIVIGTAIATCFPGSQVNLSALRTFRVFRALKAISVISGLKVIVGALLRSVKKLVDVMVLTLFCLSIFALVGQQLFMGILNQKCIKHNCGPNPASNKDCFEKEKDSEDFIMCGTWLGSRPCPNGSTCDKTTLNPDNNYTKFDNFGWSFLAMFRVMTQDSWERLYRQILRTSGIYFVFFFVVVIFLGSFYLLNLTLAVVTMAYEEQNRNVAAETEAKEKMFQEAQQLLREEKEALVAMGIDRSSLNSLQASSFSPKKRKFFGSKTRKSFFMRGSKTAQASASDSEDDASKNPQLLEQTKRLSQNLPVDLFDEHVDPLHRQRALSAVSILTITMQEQEKFQEPCFPCGKNLASKYLVWDCSPQWLCIKKVLRTIMTDPFTELAITICIIINTVFLAVEHHNMDDNLKTILKIGNWVFTGIFIAEMCLKIIALDPYHYFRHGWNVFDSIVALLSLADVLYNTLSDNNRSFLASLRVLRVFKLAKSWPTLNTLIKIIGHSVGALGNLTVVLTIVVFIFSVVGMRLFGTKFNKTAYATQERPRRRWHMDNFYHSFLVVFRILCGEWIENMWGCMQDMDGSPLCIIVFVLIMVIGKLVVLNLFIALLLNSFSNEEKDGSLEGETRKTKVQLALDRFRRAFSFMLHALQSFCCKKCRRKNSPKPKETTESFAGENKDSILPDARPWKEYDTDMALYTGQAGAPLAPLAEVEDDVEYCGEGGALPTSQHSAGVQAGDLPPETKQLTSPDDQGVEMEVFSEEDLHLSIQSPRKKSDAVSMLSECSTIDLNDIFRNLQKTVSPKKQPDRCFPKGLSCHFLCHKTDKRKSPWVLWWNIRKTCYQIVKHSWFESFIIFVILLSSGALIFEDVNLPSRPQVEKLLRCTDNIFTFIFLLEMILKWVAFGFRRYFTSAWCWLDFLIVVVSVLSLMNLPSLKSFRTLRALRPLRALSQFEGMKVVVYALISAIPAILNVLLVCLIFWLVFCILGVNLFSGKFGRCINGTDINMYLDFTEVPNRSQCNISNYSWKVPQVNFDNVGNAYLALLQVATYKGWLEIMNAAVDSREKDEQPDFEANLYAYLYFVVFIIFGSFFTLNLFIGVIIDNFNQQQKKLGGQDIFMTEEQKKYYNAMKKLGTKKPQKPIPRPLNKCQAFVFDLVTSQVFDVIILGLIVLNMIIMMAESADQPKDVKKTFDILNIAFVVIFTIECLIKVFALRQHYFTNGWNLFDCVVVVLSIISTLVSRLEDSDISFPPTLFRVVRLARIGRILRLVRAARGIRTLLFALMMSLPSLFNIGLLLFLVMFIYAIFGMSWFSKVKKGSGIDDIFNFETFTGSMLCLFQITTSAGWDTLLNPMLEAKEHCNSSSQDSCQQPQIAVVYFVSYIIISFLIVVNMYIAVILENFNTATEESEDPLGEDDFEIFYEVWEKFDPEASQFIQYSALSDFADALPEPLRVAKPNKFQFLVMDLPMVMGDRLHCMDVLFAFTTRVLGDSSGLDTMKTMMEEKFMEANPFKKLYEPIVTTTKRKEEEQGAAVIQRAYRKHMEKMVKLRLKDRSSSSHQVFCNGDLSSLDVAKVKVHND

SEQ ID NO: 168 - protein, 1790 aa, Macaca fascicularis
MDDRCYPVIFPDERNFRPFTSDSLAAIEKRIAIQKEKDKSKDKTGEVPHLRPQLDLKASRKLPNLYGDIPRELIGKPLEDLDPFYRNHKTFMVLNKKRTIYRFSAKRALFIFGPFNSIRSLAIRVSVHSLFSMFIIGTVIINCVFMARGPAKNSNSNDTDIAECVFTGIYIFEALIKILARGFILDEFSFLRDPWNWLDSIVIGIAIVSCIPGITIKLLSLRTFRVFRALKAISVVSRLKVIVGALLRSVKKLVNVIILTFFCLSIFALVGQQLFMGSLNLKCISRDCKNISNLEAYDHCFEKKENSTEFKMCGIWMVKSSCSKQYECNHTKINPDYNYTNFDNFGWSFLAMFRLMTQDSWEKLYQQTLRTAGLYSVFFFIVVIFLGSFYLINLTLAVVTMAYEEQNKNVAAEIEAKEKMFQEAQQLLKEEKEALVAMGIDRSSLTSLETSYFTPQKRKLFGNKKRKSFFLRESGKGQPPGSDSDEDSQKKPQLLEQTKRLSQNLSLDHFDEHRDPLQRQRALSAVSILTITMKEQEKSQEPCLPCGENLASKYLVWNCCPLWLCIKKVLRTVMTDPFTELAITICIIINTVFLAMEHHKMEASFEKMLNTGNLVFTSIFIAEMCLKIIALDPYHYFRRGWNIFDSIVALLSFADVMNIFQKRSWPFLRSFRVLRVFKLAKSWPTLNTLIKIIGNSVGALGSLTVVLVIVIFIFSVVGMQLFGHSFNSQKSAKLCNPTGPTVSCLRHWHMGDFWHSFLVVFRILCGEWIENMWECMQEANASSSLCVIVFILITVIGKLVVLNLFIALLLNSFSNEERNGNLEGQARKTKVQLALDRFRRAFCFVRHTLEHFCHKWCRKQKLPKQKEVTGGCAAQSKDIIPLVTEMKRGSETQEELGILTSVPKTLGIRHDRTWLAPLAEEEDDAEFSGEDNAQPITQPEAEQQAYELHQENKKPTSQGVQNVEIDMFPEDEPHLTIQDPRKKSDVTSILSECSTIDLQDGFGLLPEMVPEKQPERCLPKGFGCCFPCCSMDKRKPHWVIWWNLRKTCYQIVKHSWFESFIIFVILLSSGALIFEDVNLKDRPKIQELLNCTDIIFTHVFILEMVLKWVAFGFGKYFTSAWCCLDFIIVIVSVTTLINLKELKSFRTLRALRPLRALSQFEGMKVVVNALIGAIPAILNVLLVCLIFWLIFCILGVHFFSGKFGKCINGTESVINYTIIANKSQCESGNFSWINQKVNFDNVGNAYLALLQVATFKGWMDIIYAAVDSREKEQQPEFESNSLGYIYFVVFIVFGSFFTLNLFIGVIIDNFNQQQKKISGQDIFMTEEQKKYYNAMKKLGSKKPQKPIPRPLNKCQGLVFDVVTSQIFDIIIISLIILNMISMMAESYDQSKAVKSTLDHLNWVFVVIFTLECLIKIFALRQYYFTNGWNLFDSVVVLISIVSTMISTLESQEYIPFPPTLFRIVRLARIGRILRLVRAARGIRTLLFALMMSLPSLFNIGLLLFLIMFIYAILGMNWFSKVNPGSGIDDIFNFETFAGSMLCLFQISTSAGWDSLLSPMLRSKESCNSSSENCHLPGIATSYFVSYIIISFLIVVNMYIAVILENFNTATEESEDPLGEDDFDIFYEVWEKFDPEATQFIEYSALSDFADALPEPLRVAKPNKYQFLVMDLPMVSGDRLHCMDILFAFTARVLGGSDGLDSMKAMMEEKFMETNPLKKLYEPIVTTTKRKEEERCAAVIQKAFRKYMMKVTKGDQGDQSDLENRPHSPLQTLCNGDLPSFGVVKGKVHYD

SEQ ID NO: 169 - protein, 4 aa, Homo Sapiens
LTEF

SEQ ID NO: 170 - protein, 4 aa, Homo Sapiens
ITEF

SEQ ID NO: 171 - protein, 6 aa, Homo Sapiens
LTEFVN

SEQ ID NO: 172 - protein, 6 aa, Homo Sapiens
VTEFVD

SEQ ID NO: 173 - protein, 5 aa, Homo Sapiens
FVNLG

SEQ ID NO: 174 - protein, 5 aa, Homo Sapiens
FVDLG

SEQ ID NO: 175 - protein, 6 aa, Homo Sapiens
TTEFVD

SEQ ID NO: 176 - protein, 4 aa, Homo Sapiens
VELF

SEQ ID NO: 177 - protein, 4 aa, Homo Sapiens
MELS

SEQ ID NO: 178 - protein, 4 aa, Homo Sapiens
FLAD

SEQ ID NO: 179 - protein, 4 aa, Homo Sapiens
GLAN

SEQ ID NO: 180 - protein, 7 aa, Homo Sapiens
VELFLAD

SEQ ID NO: 181 - protein, 7 aa, Homo Sapiens
MELGLAN

SEQ ID NO: 182 - protein, 7 aa, Homo Sapiens
MELGLSN

SEQ ID NO: 183 - protein, 6 aa, Homo Sapiens
FLADVE

SEQ ID NO: 184 - protein, 6 aa, Homo Sapiens
GLANVQ

SEQ ID NO: 185 - protein, 6 aa, Homo Sapiens
TLVANT

SEQ ID NO: 186 - protein, 6 aa, Homo Sapiens
SLIANA

SEQ ID NO: 187 - protein, 5 aa, Homo Sapiens
SDLGP

SEQ ID NO: 188 - protein, 5 aa, Homo Sapiens
SELGA

SEQ ID NO: 189 - protein, 6 aa, Homo Sapiens
SLTANA

SEQ ID NO: 190 - protein, 6 aa, Homo Sapiens
SLVANA

SEQ ID NO: 191 - protein, 6 aa, Homo Sapiens
TLGYSD

SEQ ID NO: 192 - protein, 6 aa, Homo Sapiens
ALGYSE

SEQ ID NO: 193 - protein, 5 aa, Homo Sapiens
VTLVA

SEQ ID NO: 194 - protein, 5 aa, Homo Sapiens
ISLVA

SEQ ID NO: 195 - protein, 6 aa, Homo Sapiens
SLVANW

SEQ ID NO: 196 - protein, 5 aa, Homo Sapiens
TAIDL

SEQ ID NO: 197 - protein, 6 aa, Homo Sapiens
WLGYSE

SEQ ID NO: 198 - protein, 5 aa, Homo Sapiens
GYSDL

SEQ ID NO: 199 - protein, 5 aa, Homo Sapiens
GFAEM

SEQ ID NO: 200 - protein, 5 aa, Homo Sapiens
TLVAN

SEQ ID NO: 201 - protein, 5 aa, Homo Sapiens
SLVAN

SEQ ID NO: 202 - protein, 5 aa, Homo Sapiens
ISLTA

SEQ ID NO: 203 - protein, 6 aa, Homo Sapiens
VANTLG

SEQ ID NO: 204 - protein, 6 aa, Homo Sapiens
TAKILE

SEQ ID NO: 205 - protein, 6 aa, Homo Sapiens
GYSDLG

SEQ ID NO: 206 - protein, 6 aa, Homo Sapiens
EYSEVA

SEQ ID NO: 207 - protein, 5 aa, Homo Sapiens
TTLIN

SEQ ID NO: 208 - protein, 4 aa, Homo Sapiens
LGPI

SEQ ID NO: 209 - protein, 4 aa, Homo Sapiens
LMEL

SEQ ID NO: 210 - protein, 4 aa, Homo Sapiens
LIET

SEQ ID NO: 211 - protein, 4 aa, Homo Sapiens
IIEK

SEQ ID NO: 212 - protein, 5 aa, Homo Sapiens
DLIET

SEQ ID NO: 213 - protein, 5 aa, Homo Sapiens
ELIEK

SEQ ID NO: 214 - protein, 5 aa, Homo Sapiens
EMIEK

SEQ ID NO: 215 - protein, 5 aa, Homo Sapiens
MFLAD

SEQ ID NO: 216 - protein, 5 aa, Homo Sapiens
LALSD

SEQ ID NO: 217 - protein, 4 aa, Homo Sapiens
LIQK

SEQ ID NO: 218 - protein, 5 aa, Homo Sapiens
ADLIE

SEQ ID NO: 219 - protein, 5 aa, Homo Sapiens
SDLIQ

SEQ ID NO: 220 - protein, 5 aa, Homo Sapiens
TVLSD

SEQ ID NO: 221 - protein, 6 aa, Homo Sapiens
ADLIET

SEQ ID NO: 222 - protein, 6 aa, Homo Sapiens
SDIIQK

SEQ ID NO: 223 - protein, 5 aa, Homo Sapiens
ETYFV

SEQ ID NO: 224 - protein, 5 aa, Homo Sapiens
QKYFF

SEQ ID NO: 225 - protein, 12 aa, Homo Sapiens
MFLADLIETYFV

SEQ ID NO: 226 - protein, 12 aa, Homo Sapiens
TVLSDIIQKYFF

SEQ ID NO: 227 - protein, 12 aa, Homo Sapiens
VTLVANTLGYSD

SEQ ID NO: 228 - protein, 12 aa, Homo Sapiens
ISLVANWLGYSE

SEQ ID NO: 229 - protein, 9 aa, Homo Sapiens
MFLADLIET

SEQ ID NO: 230 - protein, 9 aa, Homo Sapiens
LALSDLIQK

SEQ ID NO: 231 - protein, 14 aa, Homo Sapiens
TLVANTLGYSDLGP

SEQ ID NO: 232 - protein, 13 aa, Homo Sapiens
SLVANAGYSELGA

SEQ ID NO: 233 - protein, 14 aa, Homo Sapiens
SLTANALGYSELGA

SEQ ID NO: 234 - protein, 6 aa, Homo Sapiens
VGTAID

SEQ ID NO: 235 - protein, 6 aa, Homo Sapiens
ITEFVN

SEQ ID NO: 236 - protein, 5 aa, Homo Sapiens
IDLRG

SEQ ID NO: 237 - protein, 5 aa, Homo Sapiens
VNLGN

SEQ ID NO: 238 - protein, 5 aa, Homo Sapiens
VDLGN

SEQ ID NO: 239 - protein, 6 aa, Homo Sapiens
LTEFVD

SEQ ID NO: 240 - protein, 4 aa, Homo Sapiens
VTEF

SEQ ID NO: 241 - protein, 4 aa, Homo Sapiens
LELG

SEQ ID NO: 242 - protein, 6 aa, Homo Sapiens
VAKKGS

SEQ ID NO: 243 - protein, 6 aa, Homo Sapiens
LADVEG

SEQ ID NO: 244 - protein, 5 aa, Homo Sapiens
LGVAK

SEQ ID NO: 245 - protein, 5 aa, Homo Sapiens
LSLAD

SEQ ID NO: 246 - protein, 5 aa, Homo Sapiens
LELGV

SEQ ID NO: 247 - protein, 5 aa, Homo Sapiens
VELGL

SEQ ID NO: 248 - protein, 6 aa, Homo Sapiens
LANVEG

SEQ ID NO: 249 - protein, 5 aa, Homo Sapiens
GVAKK

SEQ ID NO: 250 - protein, 5 aa, Homo Sapiens
GLANV

SEQ ID NO: 251 - protein, 5 aa, Homo Sapiens
MELSL

SEQ ID NO: 252 - protein, 5 aa, Homo Sapiens
MELGL

SEQ ID NO: 253 - protein, 4 aa, Homo Sapiens
MELG

SEQ ID NO: 254 - protein, 6 aa, Homo Sapiens
LSNVEG

SEQ ID NO: 255 - protein, 5 aa, Homo Sapiens
GLSNV

SEQ ID NO: 256 - protein, 6 aa, Homo Sapiens
LANVQG

SEQ ID NO: 257 - protein, 5 aa, Homo Sapiens
GLSRM

SEQ ID NO: 258 - protein, 6 aa, Homo Sapiens
LSRMSN

SEQ ID NO: 259 - protein, 5 aa, Homo Sapiens
FLADV

SEQ ID NO: 260 - protein, 7 aa, Homo Sapiens
ADVMNCV

SEQ ID NO: 261 - protein, 6 aa, Homo Sapiens
VLQKRS

SEQ ID NO: 262 - protein, 5 aa, Homo Sapiens
VSLIA

SEQ ID NO: 263 - protein, 6 aa, Homo Sapiens
IANALG

SEQ ID NO: 264 - protein, 5 aa, Homo Sapiens
SEVAP

SEQ ID NO: 265 - protein, 6 aa, Homo Sapiens
SLTAKI

SEQ ID NO: 266 - protein, 5 aa, Homo Sapiens
KILEY

SEQ ID NO: 267 - protein, 5 aa, Homo Sapiens
NALGY

SEQ ID NO: 268 - protein, 5 aa, Homo Sapiens
VSLTA

SEQ ID NO: 269 - protein, 6 aa, Homo Sapiens
TANALG

SEQ ID NO: 270 - protein, 5 aa, Homo Sapiens
VSLVA

SEQ ID NO: 271 - protein, 6 aa, Homo Sapiens
VANALG

SEQ ID NO: 272 - protein, 6 aa, Homo Sapiens
VANWLG

SEQ ID NO: 273 - protein, 5 aa, Homo Sapiens
SELGP

SEQ ID NO: 274 - protein, 5 aa, Homo Sapiens
NWLGY

SEQ ID NO: 275 - protein, 5 aa, Homo Sapiens
LMELK

SEQ ID NO: 276 - protein, 5 aa, Homo Sapiens
AEMGP

SEQ ID NO: 277 - protein, 6 aa, Homo Sapiens
SLVANT

SEQ ID NO: 278 - protein, 6 aa, Homo Sapiens
ANTLGF

SEQ ID NO: 279 - protein, 6 aa, Homo Sapiens
ANTLGY

SEQ ID NO: 280 - protein, 4 aa, Homo Sapiens
YSEV

SEQ ID NO: 281 - protein, 5 aa, Homo Sapiens
LGAIK

SEQ ID NO: 282 - protein, 7 aa, Homo Sapiens
ASLIFSA

SEQ ID NO: 283 - protein, 7 aa, Homo Sapiens
VGMFLAD

SEQ ID NO: 284 - protein, 7 aa, Homo Sapiens
IFSAILK

SEQ ID NO: 285 - protein, 7 aa, Homo Sapiens
FLADIIE

SEQ ID NO: 286 - protein, 7 aa, Homo Sapiens
VGMFLAE

SEQ ID NO: 287 - protein, 7 aa, Homo Sapiens
FLAELIE

SEQ ID NO: 288 - protein, 7 aa, Homo Sapiens
FLAEMIE

SEQ ID NO: 289 - protein, 7 aa, Homo Sapiens
VGLALSD

SEQ ID NO: 290 - protein, 7 aa, Homo Sapiens
ALSDLIQ

SEQ ID NO: 291 - protein, 7 aa, Homo Sapiens
VGTVLSD

SEQ ID NO: 292 - protein, 7 aa, Homo Sapiens
VLSDIIQ

SEQ ID NO: 293 - protein, 7 aa, Homo Sapiens
FLADLIE

SEQ ID NO: 294 - protein, 7 aa, Homo Sapiens
VSTMIST

SEQ ID NO: 295 - protein, 7 aa, Homo Sapiens
MISTLEN

SEQ ID NO: 296 - protein, 6 aa, Homo Sapiens
QSYFSP

SEQ ID NO: 297 - protein, 6 aa, Homo Sapiens
HIPFPP

SEQ ID NO: 298 - protein, 5 aa, Homo Sapiens
VSYIP

SEQ ID NO: 299 - protein, 5 aa, Homo Sapiens
ITEFV

SEQ ID NO: 300 - protein, 5 aa, Homo Sapiens
EFVNL

SEQ ID NO: 301 - protein, 10 aa, Homo Sapiens
YIPGIVTEFV

SEQ ID NO: 302 - protein, 5 aa, Homo Sapiens
GITIK

SEQ ID NO: 303 - protein, 5 aa, Homo Sapiens
VTEFV

SEQ ID NO: 304 - protein, 5 aa, Homo Sapiens
EFVDL

SEQ ID NO: 305 - protein, 5 aa, Homo Sapiens
LTEFV

SEQ ID NO: 306 - protein, 5 aa, Homo Sapiens
TTEFV

SEQ ID NO: 307 - protein, 5 aa, Homo Sapiens
VGTAI

SEQ ID NO: 308 - protein, 5 aa, Homo Sapiens
LGPIK

SEQ ID NO: 309 - protein, 5 aa, Homo Sapiens
MGPIK

SEQ ID NO: 310 - protein, 5 aa, Homo Sapiens
VAPIK

SEQ ID NO: 311 - protein, 6 aa, Homo Sapiens
KYFVSP

SEQ ID NO: 312 - protein, 9 aa, Homo Sapiens
STMISTLEN

SEQ ID NO: 313 - protein, 9 aa, Homo Sapiens
GMFLADIIE

SEQ ID NO: 314 - protein, 9 aa, Homo Sapiens
GMFLAELIE

SEQ ID NO: 315 - protein, 9 aa, Homo Sapiens
GMFLAEMIE

SEQ ID NO: 316 - protein, 6 aa, Homo Sapiens
KYFFSP

SEQ ID NO: 317 - protein, 10 aa, Homo Sapiens
VGTVLSDIIQ

SEQ ID NO: 318 - protein, 6 aa, Homo Sapiens
TYFVSP

SEQ ID NO: 319 - protein, 9 aa, Homo Sapiens
GMFLADLIE

SEQ ID NO: 320 - protein, 5 aa, Homo Sapiens
SLIAN

SEQ ID NO: 321 - protein, 10 aa, Homo Sapiens
ASLIFSAILK

SEQ ID NO: 322 - protein, 9 aa, Homo Sapiens
GLALSDLIQ

SEQ ID NO: 323 - protein, 5 aa, Homo Sapiens
SLTAN

SEQ ID NO: 324 - protein, 5 aa, Homo Sapiens
SLTAK

SEQ ID NO: 325 - protein, 12 aa, Homo Sapiens
TLVANTLGYSDL

SEQ ID NO: 326 - protein, 12 aa, Homo Sapiens
SLVANTLGFAEM

SEQ ID NO: 327 - protein, 14 aa, Homo Sapiens
SLIANALGYSELGA

SEQ ID NO: 328 - protein, 6 aa, Homo Sapiens
ILEYSE

SEQ ID NO: 329 - protein, 5934 aa, Homo Sapiens
ATGGCAATGTTGCCTCCCCCAGGACCTCAGAGCTTTGTCCATTTCACAAAACAGTCTCTTGCCCTCATTGAACAACGCATTGCTGAAAGAAAATCAAAGGAACCCAAAGAAGAAAAGAAAGATGATGATGAAGAAGCCCCAAAGCCAAGCAGTGACTTGGAAGCTGGCAAACAGCTGCCCTTCATCTATGGGGACATTCCTCCCGGCATGGTGTCAGAGCCCCTGGAGGACTTGGACCCCTACTATGCAGACAAAAAGACTTTCATAGTATTGAACAAAGGGAAAACAATCTTCCGTTTCAATGCCACACCTGCTTTATATATGCTTTCTCCTTTCAGTCCTCTAAGAAGAATATCTATTAAGATTTTAGTACACTCCTTATTCAGCATGCTCATCATGTGCACTATTCTGACAAACTGCATATTTATGACCATGAATAACCCACCGGACTGGACCAAAAATGTCGAGTACACTTTTACTGGAATATATACTTTTGAATCACTTGTAAAAATCCTTGCAAGAGGCTTCTGTGTAGGAGAATTCACTTTTCTTCGTGACCCGTGGAACTGGCTGGATTTTGTCGTCATTGTTTTTGCGTATTTAACAGAATTTGTAAACCTAGGCAATGTTTCAGCTCTTCGAACTTTCAGAGTATTGAGAGCTTTGAAAACTATTTCTGTAATCCCAGGCCTGAAGACAATTGTAGGGGCTTTGATCCAGTCAGTGAAGAAGCTTTCTGATGTCATGATCCTGACTGTGTTCTGTCTGAGTGTGTTTGCACTAATTGGACTACAGCTGTTCATGGGAAACCTGAAGCATAAATGTTTTCGAAATTCACTTGAAAATAATGAAACATTAGAAAGCATAATGAATACCCTAGAGAGTGAAGAAGACTTTAGAAAATATTTTTATTACTTGGAAGGATCCAAAGATGCTCTCCTTTGTGGTTTCAGCACAGATTCAGGTCAGTGTCCAGAGGGGTACACCTGTGTGAAAATTGGCAGAAACCCTGATTATGGCTACACGAGCTTTGACACTTTCAGCTGGGCCTTCTTAGCCTTGTTTAGGCTAATGACCCAAGATTACTGGGAAAACCTTTACCAACAGACGCTGCGTGCTGCTGGCAAAACCTACATGATCTTCTTTGTCGTAGTGATTTTCCTGGGCTCCTTTTATCTAATAAACTTGATCCTGGCTGTGGTTGCCATGGCATATGAAGAACAGAACCAGGCAAACATTGAAGAAGCTAAACAGAAAGAATTAGAATTTCAACAGATGTTAGACCGTCTTAAAAAAGAGCAAGAAGAAGCTGAGGCAATTGCAGCGGCAGCGGCTGAATATACAAGTATTAGGAGAAGCAGAATTATGGGCCTCTCAGAGAGTTCTTCTGAAACATCCAAACTGAGCTCTAAAAGTGCTAAAGAAAGAAGAAACAGAAGAAAGAAAAAGAATCAAAAGAAGCTCTCCAGTGGAGAGGAAAAGGGAGATGCTGAGAAATTGTCGAAATCAGAATCAGAGGACAGCATCAGAAGAAAAAGTTTCCACCTTGGTGTCGAAGGGCATAGGCGAGCACATGAAAAGAGGTTGTCTACCCCCAATCAGTCACCACTCAGCATTCGTGGCTCCTTGTTTTCTGCAAGGCGAAGCAGCAGAACAAGTCTTTTTAGTTTCAAAGGCAGAGGAAGAGATATAGGATCTGAGACTGAATTTGCCGATGATGAGCACAGCATTTTTGGAGACAATGAGAGCAGAAGGGGCTCACTGTTTGTGCCCCACAGACCCCAGGAGCGACGCAGCAGTAACATCAGCCAAGCCAGTAGGTCCCCACCAATGCTGCCGGTGAACGGGAAAATGCACAGTGCTGTGGACTGCAACGGTGTGGTCTCCCTGGTTGATGGACGCTCAGCCCTCATGCTCCCCAATGGACAGCTTCTGCCAGAGGGCACGACCAATCAAATACACAAGAAAAGGCGTTGTAGTTCCTATCTCCTTTCAGAGGATATGCTGAATGATCCCAACCTCAGACAGAGAGCAATGAGTAGAGCAAGCATATTAACAAACACTGTGGAAGAACTTGAAGAGTCCAGACAAAAATGTCCACCTTGGTGGTACAGATTTGCACACAAATTCTTGATCTGGAATTGCTCTCCATATTGGATAAAATTCAAAAAGTGTATCTATTTTATTGTAATGGATCCTTTTGTAGATCTTGCAATTACCATTTGCATAGTTTTAAACACATTATTTATGGCTATGGAACACCACCCAATGACTGAGGAATTCAAAAATGTACTTGCTATAGGAAATTTGGTCTTTACTGGAATCTTTGCAGCTGAAATGGTATTAAAACTGATTGCCATGGATCCATATGAGTATTTCCAAGTAGGCTGGAATATTTTTGACAGCCTTATTGTGACTTTAAGTTTAGTGGAGCTCTTTCTAGCAGATGTGGAAGGATTGTCAGTTCTGCGATCATTCAGACTGCTCCGAGTCTTCAAGTTGGCAAAATCCTGGCCAACATTGAACATGCTGATTAAGATCATTGGTAACTCAGTAGGGGCTCTAGGTAACCTCACCTTAGTGTTGGCCATCATCGTCTTCATTTTTGCTGTGGTCGGCATGCAGCTCTTTGGTAAGAGCTACAAAGAATGTGTCTGCAAGATCAATGATGACTGTACGCTCCCACGGTGGCACATGAACGACTTCTTCCACTCCTTCCTGATTGTGTTCCGCGTGCTGTGTGGAGAGTGGATAGAGACCATGTGGGACTGTATGGAGGTCGCTGGTCAAGCTATGTGCCTTATTGTTTACATGATGGTCATGGTCATTGGAAACCTGGTGGTCCTAAACCTATTTCTGGCCTTATTATTGAGCTCATTTAGTTCAGACAATCTTACAGCAATTGAAGAAGACCCTGATGCAAACAACCTCCAGATTGCAGTGACTAGAATTAAAAAGGGAATAAATTATGTGAAACAAACCTTACGTGAATTTATTCTAAAAGCATTTTCCAAAAAGCCAAAGATTTCCAGGGAGATAAGACAAGCAGAAGATCTGAATACTAAGAAGGAAAACTATATTTCTAACCATACACTTGCTGAAATGAGCAAAGGTCACAATTTCCTCAAGGAAAAAGATAAAATCAGTGGTTTTGGAAGCAGCGTGGACAAACACTTGATGGAAGACAGTGATGGTCAATCATTTATTCACAATCCCAGCCTCACAGTGACAGTGCCAATTGCACCTGGGGAATCCGATTTGGAAAATATGAATGCTGAGGAACTTAGCAGTGATTCGGATAGTGAATACAGCAAAGTGAGATTAAACCGGTCAAGCTCCTCAGAGTGCAGCACAGTTGATAACCCTTTGCCTGGAGAAGGAGAAGAAGCAGAGGCTGAACCTATGAATTCCGATGAGCCAGAGGCCTGTTTCACAGATGGTTGTGTACGGAGGTTCTCATGCTGCCAAGTTAACATAGAGTCAGGGAAAGGAAAAATCTGGTGGAACATCAGGAAAACCTGCTACAAGATTGTTGAACACAGTTGGTTTGAAAGCTTCATTGTCCTCATGATCCTGCTCAGCAGTGGTGCCCTGGCTTTTGAAGATATTTATATTGAAAGGAAAAAGACCATTAAGATTATCCTGGAGTATGCAGACAAGATCTTCACTTACATCTTCATTCTGGAAATGCTTCTAAAATGGATAGCATATGGTTATAAAACATATTTCACCAATGCCTGGTGTTGGCTGGATTTCCTAATTGTTGATGTTTCTTTGGTTACTTTAGTGGCAAACACTCTTGGCTACTCAGATCTTGGCCCCATTAAATCCCTTCGGACACTGAGAGCTTTAAGACCTCTAAGAGCCTTATCTAGATTTGAAGGAATGAGGGTCGTTGTGAATGCACTCATAGGAGCAATTCCTTCCATCATGAATGTGCTACTTGTGTGTCTTATATTCTGGCTGATATTCAGCATCATGGGAGTAAATTTGTTTGCTGGCAAGTTCTATGAGTGTATTAACACCACAGATGGGTCACGGTTTCCTGCAAGTCAAGTTCCAAATCGTTCCGAATGTTTTGCCCTTATGAATGTTAGTCAAAATGTGCGATGGAAAAACCTGAAAGTGAACTTTGATAATGTCGGACTTGGTTACCTATCTCTGCTTCAAGTTGCAACTTTTAAGGGATGGACGATTATTATGTATGCAGCAGTGGATTCTGTTAATGTAGACAAGCAGCCCAAATATGAATATAGCCTCTACATGTATATTTATTTTGTCGTCTTTATCATCTTTGGGTCATTCTTCACTTTGAACTTGTTCATTGGTGTCATCATAGATAATTTCAACCAACAGAAAAAGAAGCTTGGAGGTCAAGACATCTTTATGACAGAAGAACAGAAGAAATACTATAATGCAATGAAAAAGCTGGGGTCCAAGAAGCCACAAAAGCCAATTCCTCGACCAGGGAACAAAATCCAAGGATGTATATTTGACCTAGTGACAAATCAAGCCTTTGATATTAGTATCATGGTTCTTATCTGTCTCAACATGGTAACCATGATGGTAGAAAAGGAGGGTCAAAGTCAACATATGACTGAAGTTTTATATTGGATAAATGTGGTTTTTATAATCCTTTTCACTGGAGAATGTGTGCTAAAACTGATCTCCCTCAGACACTACTACTTCACTGTAGGATGGAATATTTTTGATTTTGTGGTTGTGATTATCTCCATTGTAGGTATGTTTCTAGCTGATTTGATTGAAACGTATTTTGTGTCCCCTACCCTGTTCCGAGTGATCCGTCTTGCCAGGATTGGCCGAATCCTACGTCTAGTCAAAGGAGCAAAGGGGATCCGCACGCTGCTCTTTGCTTTGATGATGTCCCTTCCTGCGTTGTTTAACATCGGCCTCCTGCTCTTCCTGGTCATGTTCATCTACGCCATCTTTGGAATGTCCAACTTTGCCTATGTTAAAAAGGAAGATGGAATTAATGACATGTTCAATTTTGAGACCTTTGGCAACAGTATGATTTGCCTGTTCCAAATTACAACCTCTGCTGGCTGGGATGGATTGCTAGCACCTATTCTTAACAGTAAGCCACCCGACTGTGACCCAAAAAAAGTTCATCCTGGAAGTTCAGTTGAAGGAGACTGTGGTAACCCATCTGTTGGAATATTCTACTTTGTTAGTTATATCATCATATCCTTCCTGGTTGTGGTGAACATGTACATTGCAGTCATACTGGAGAATTTTAGTGTTGCCACTGAAGAAAGTACTGAACCTCTGAGTGAGGATGACTTTGAGATGTTCTATGAGGTTTGGGAGAAGTTTGATCCCGATGCGACCCAGTTTATAGAGTTCTCTAAACTCTCTGATTTTGCAGCTGCCCTGGATCCTCCTCTTCTCATAGCAAAACCCAACAAAGTCCAGCTCATTGCCATGGATCTGCCCATGGTTAGTGGTGACCGGATCCATTGTCTTGACATCTTATTTGCTTTTACAAAGCGTGTTTTGGGTGAGAGTGGGGAGATGGATTCTCTTCGTTCACAGATGGAAGAAAGGTTCATGTCTGCAAATCCTTCCAAAGTGTCCTATGAACCCATCACAACCACACTAAAACGGAAACAAGAGGATGTGTCTGCTACTGTCATTCAGCGTGCTTATAGACGTTACCGCTTAAGGCAAAATGTCAAAAATATATCAAGTATATACATAAAAGATGGAGACAGAGATGATGATTTACTCAATAAAAAAGATATGGCTTTTGATAATGTTAATGAGAACTCAAGTCCAGAAAAAACAGATGCCACTTCATCCACCACCTCTCCACCTTCATATGATAGTGTAACAAAGCCAGACAAAGAGAAATATGAACAAGACAGAACAGAAAAGGAAGACAAAGGGAAAGACAGCAAGGAAAGCAAAAAATAG

SEQ ID NO: 330 - protein, 166 aa, Homo Sapiens
ACCTGGGAAACATCCGCGAGCACAGCCTACATGGACCTGAGTAGTCTGAGATCGGAAGACACGGCTGTTTATTACTGTGCGAGAGGGTTTACTATGGTTCGGGGAGCCCCCTATTATGACGGTATGGACGTCTGGGGCCAAGGGACCACGGTCACCGTCTCCTCAG

SEQ ID NO: 331 - protein, 125 aa, Homo Sapiens
QVQLVQSGAEVRKPGASVKVSCKASGYTFTDYAIHWVRQAPGQRLEWMGWIIVGNGKTRYSQKFQGRVTITWETSASTAYMDLSSLRSEDTAVYYCARGFTMVRGAPYYDGMDVWGQGTTVTVSS

SEQ ID NO: 332 - protein, 334 aa, Homo Sapiens
GATATTGTGATGACTCAGTCTCCACTCTCCCTGCCCGTCACCCCTGGAGAGCCGGCCTCCATCTCCTGCAGGTCTAGTCAGAGCCTCCTGTATAGTACTGGATACAACTATTTGGATTGGTACCTGCAGAAGCCAGGGCTGTCTCCACAGCTCCTGATCTATTTGGGTTCTAATCGGGCCTCCGGGGTCCCTGACAGGTTCAGTGGCAGTGGATCAGGCACAGATTTTACACTGAAAATCAGCAGAGTGGAGGCTGAGGATGTTGGGGTTTATTACTGCATGCAAGCTCTACAAACTCCCACTTTCGGCGGAGGGACCAAGGTGGAGATCAAAC

SEQ ID NO: 333 - protein, 111 aa, Homo Sapiens
DIVMTQSPLSLPVTPGEPASISCRSSQSLLYSTGYNYLDWYLQKPGLSPQLLIYLGSNRASGVPDRFSGSGSGTDFTLKISRVEAEDVGVYYCMQALQTPTFGGGTKVEIK

SEQ ID NO: 334 - protein, 5 aa, Homo Sapiens
DYAIH

SEQ ID NO: 335 - protein, 8 aa, Homo Sapiens
GYTFTDYA

SEQ ID NO: 336 - protein, 17 aa, Homo Sapiens
WIIVGNGKTRYSQKFQG

SEQ ID NO: 337 - protein, 8 aa, Homo Sapiens
IIVGNGKT

SEQ ID NO: 338 - protein, 16 aa, Homo Sapiens
GFTMVRGAPYYDGMDV

SEQ ID NO: 339 - protein, 18 aa, Homo Sapiens
ARGFTMVRGAPYYDGMDV

SEQ ID NO: 340 - protein, 16 aa, Homo Sapiens
RSSQSLLYSTGYNYLD

SEQ ID NO: 341 - protein, 11 aa, Homo Sapiens
QSLLYSTGYNY

SEQ ID NO: 342 - protein, 7 aa, Homo Sapiens
LGSNRAS

SEQ ID NO: 343 - protein, 3 aa, Homo Sapiens
LGS

SEQ ID NO: 344 - protein, 8 aa, Homo Sapiens
MQALQTPT

SEQ ID NO: 345 - protein, 8 aa, Homo Sapiens
MQALQTPT

SEQ ID NO: 346 - DNA, 376 bp, Homo Sapiens
caggtccagcttgtgcagtctggggcagaggtgaggaagcctggggcctcagtgaaggtttcctgcaaggcttctggatacaccttcactgactatgcaatacattgggtgcgccaggcccccggacaaaggcttgagtggatgggatggatcatcgttggcaatggtaagacaagatatgcacagaagttccagggcagagtcaccattacctgggaaacatccgcgaccacagtctacatggacctgagtagtctgagatcggaagacacggctgtttattactgtgcgagagggtttactatggttcggggagccccctattatgacggtttggacgtctggggccaagggaccacggtcaccgtctcctcag

SEQ ID NO: 347 - protein, 125 aa, Homo Sapiens
QVQLVQSGAEVRKPGASVKVSCKASGYTFTDYAIHWVRQAPGQRLEWMGWIIVGNGKTRYAQKFQGRVTITWETSATTVYMDLSSLRSEDTAVYYCARGFTMVRGAPYYDGLDVWGQGTTVTVSS

SEQ ID NO: 348 - DNA, 334 bp, Homo Sapiens
gatattgtgatgactcagtctccactctccctgcccgtcacccctggagagccggcctccatctcctgcaggtctagtcagagcctcctgtatagtactggatacaactatttggattggtacctgcagaagccagggctgtctccacagctcctgatctatttgggttctaatcgggcctccggggtccctgacaggttcagtggcagtggatcaggcacagaatttacactgaaaatcagcagagtggaggctgaggatgttggggtttattactgcatgcaagctctacaaactcccactttcggcggagggaccaaggtggagatcaaac

SEQ ID NO: 349 - protein, 111 aa, Homo Sapiens; identical to SEQ ID NO: 333

SEQ ID NO: 350 - protein, 5 aa, Homo Sapiens
DYAIH

SEQ ID NO: 351 - protein, 8 aa, Homo Sapiens
GYTFTDYA

SEQ ID NO: 352 - protein, 17 aa, Homo Sapiens
WIIVGNGKTRYAQKFQG

SEQ ID NO: 353 - protein, 8 aa, Homo Sapiens
IIVGNGKT

SEQ ID NO: 354 - protein, 16 aa, Homo Sapiens
GFTMVRGAPYYDGLDV

SEQ ID NO: 355 - protein, 18 aa, Homo Sapiens
ARGFTMVRGAPYYDGLDV

SEQ ID NO: 356 - protein, 16 aa, Homo Sapiens
RSSQSLLYSTGYNYLD

SEQ ID NO: 357 - protein, 11 aa, Homo Sapiens
QSLLYSTGYNY

SEQ ID NO: 358 - protein, 7 aa, Homo Sapiens
LGSNRAS

SEQ ID NO: 359 - protein, 3 aa, Homo Sapiens
LGS

SEQ ID NO: 360 - protein, 8 aa, Homo Sapiens
MQALQTPT

SEQ ID NO: 361 - protein, 8 aa, Homo Sapiens
MQALQTPT

SEQ ID NO: 362 - DNA, 376 bp, Homo Sapiens
caggtccagcttgtgcagtctggggctgaggtgaggaagcctggggcctcagtgaaagtttcctgcaaggcttctggatacaccttcactgactatgcaatacattgggtgcgccaggcccccggacaaaggcttgagtggatgggatggatcatcgttggcaatggtaagacaagatattcacagaagtttcagggcagagtcaccattacctgggaaacatccgcgaccacagcctacatggacctgagtagtctgagatcggaggacacggctgtttattactgtgcgagagggtttactatggttcggggagccccctattatgacggtatggacgtctggggccaagggaccacggtcaccgtctcctcag

SEQ ID NO: 363 - protein, 125 aa, Homo Sapiens
QVQLVQSGAEVRKPGASVKVSCKASGYTFTDYAIHWVRQAPGQRLEWMGWIIVGNGKTRYSQKFQGRVTITWETSATTAYMDLSSLRSEDTAVYYCARGFTMVRGAPYYDGMDVWGQGTTVTVSS

SEQ ID NO: 364 - DNA, 334 bp, Homo Sapiens
gatattgtgatgactcagtctccactctccctgcccgtcacccctggagagccggcctccatctcctgcaggtctagtcagagcctcctgtatactactggatacaactatttggattggtacctgcagaagccagggctgtctccacagctcctgatctatttgggttcttatcgggcctccggggtccctgacaggttcactggcagtggatcaggcacagattttacactgaagatcagcagagtggaggctgaggatgttggggtttattactgcatgcaagctctacaagctcctactttcggcggagggaccaagttggagatcaaac

SEQ ID NO: 365 - protein, 111 aa, Homo Sapiens
DIVMTQSPLSLPVTPGEPASISCRSSQSLLYTTGYNYLDWYLQKPGLSPQLLIYLGSYRASGVPDRFTGSGSGTDFTLKISRVEAEDVGVYYCMQALQAPTFGGGTKLEIK

SEQ ID NO: 366 - protein, 5 aa, Homo Sapiens
DYAIH

SEQ ID NO: 367 - protein, 8 aa, Homo Sapiens
GYTFTDYA

SEQ ID NO: 368 - protein, 17 aa, Homo Sapiens
WIIVGNGKTRYSQKFQG

SEQ ID NO: 369 - protein, 8 aa, Homo Sapiens
IIVGNGKT

SEQ ID NO: 370 - protein, 16 aa, Homo Sapiens
GFTMVRGAPYYDGMDV

SEQ ID NO: 371 - protein, 18 aa, Homo Sapiens
ARGFTMVRGAPYYDGMDV

SEQ ID NO: 372 - protein, 16 aa, Homo Sapiens
RSSQSLLYTTGYNYLD

SEQ ID NO: 373 - protein, 11 aa, Homo Sapiens
QSLLYTTGYNY

SEQ ID NO: 374 - protein, 7 aa, Homo Sapiens
LGSYRAS

SEQ ID NO: 375 - protein, 3 aa, Homo Sapiens
LGS

SEQ ID NO: 376 - protein, 8 aa, Homo Sapiens
MQALQAPT

SEQ ID NO: 377 - protein, 8 aa, Homo Sapiens
MQALQAPT

SEQ ID NO: 378 - DNA, 349 bp, Homo Sapiens
caggttcaattggtgcaatctgaagctgaggtgaagaagcccggggcctcagtgaaggtctcctgcaaggcttctggttatatctttaccacctttggtctcagctgggtgcgacaggcccctggacaaggacttgagtggatgggaaggatcaccacaaataatgggaacacaatctatgagaggagattccagggcagagtcaccatgaccatagacacatccacgacaactgcctacatggagatgagaagcctgacatctgacgatacggccgtttattattgtgcgagagatggggccccacaggaccactggggccagggaaccctggtcaccgtctcctcag

SEQ ID NO: 379 - protein, 116 aa, Homo Sapiens
QVQLVQSEAEVKKPGASVKVSCKASGYIFTTFGLSWVRQAPGQGLEWMGRITTNNGNTIYERRFQGRVTMTIDTSTTTAYMEMRSLTSDDTAVYYCARDGAPQDHWGQGTLVTVSS

SEQ ID NO: 380 - DNA, 322 bp, Homo Sapiens
gacatccagatgacccagtctccatcctcactgtctgtctctgtgggagacagagtcatcatcacctgtcgggcgagtcaagacattagaaattctttagcctggtttcagcaaaaacctgggaaagcccctaagtccctgatctttgctgcatccagtttgcaaagtggggtcccatcaaagttccgcggcagtggatttgggacagatttcactctcaccatcaccagcctgcagcctgaggattttgcaacttattactgccaacagtatagtagttatcctctcacttttggcggagggaccaaggtagagatcaaac

SEQ ID NO: 381 - protein, 107 aa, Homo Sapiens
DIQMTQSPSSLSVSVGDRVIITCRASQDIRNSLAWFQQKPGKAPKSLIFAASSLQSGVPSKFRGSGFGTDFTLTITSLQPEDFATYYCQQYSSYPLTFGGGTKVEIK

SEQ ID NO: 382 - protein, 5 aa, Homo Sapiens
TFGLS

SEQ ID NO: 383 - protein, 8 aa, Homo Sapiens
GYIFTTFG

SEQ ID NO: 384 - protein, 17 aa, Homo Sapiens
RITTNNGNTIYERRFQG

SEQ ID NO: 385 - protein, 8 aa, Homo Sapiens
ITTNNGNT

SEQ ID NO: 386 - protein, 7 aa, Homo Sapiens
DGAPQDH

SEQ ID NO: 387 - protein, 9 aa, Homo Sapiens
ARDGAPQDH

SEQ ID NO: 388 - protein, 11 aa, Homo Sapiens
RASQDIRNSLA

SEQ ID NO: 389 - protein, 6 aa, Homo Sapiens
QDIRNS

SEQ ID NO: 390 - protein, 7 aa, Homo Sapiens
AASSLQS

SEQ ID NO: 391 - protein, 3 aa, Homo Sapiens
AAS

SEQ ID NO: 392 - protein, 9 aa, Homo Sapiens
QQYSSYPLT

SEQ ID NO: 393 - protein, 9 aa, Homo Sapiens
QQYSSYPLT

SEQ ID NO: 394 - DNA, 358 bp, Homo Sapiens
gaggtgcacttggtagaatctgggggaggcttggttcagccgggggggtccctgagactctcctgttcagcctctagattcacctttagcacctctgccatgacctgggtccgccaggctccagggaaggggctggagtgggtctcagttattagtgctagtggtactaccacatattacggagactccgtgaagggccggttcaccatctccagagacaattccaagaacacgctacatctgcaaatgaacagcctgagagccgaggacacggccgtatattattgtgcgaaagggggtttggcagtgcctggtccgaactactggggccagggaaccctggtcaccgtctcctcag

SEQ ID NO: 395 - protein, 119 aa, Homo Sapiens
EVHLVESGGGLVQPGGSLRLSCSASRFTFSTSAMTWVRQAPGKGLEWVSVISASGTTTYYGDSVKGRFTISRDNSKNTLHLQMNSLRAEDTAVYYCAKGGLAVPGPNYWGQGTLVTVSS

SEQ ID NO: 396 - DNA, 337 bp, Homo Sapiens
gatgttgtgatgactcagtctccactctccctgcccgtcacccttggacagccggcctccatctcctgcaggtccagtcaaagcctcgtcttcagtgatggaaacacctacttgacttggtttcaacagaggccaggccaatctccaaggcgcctaatttataaggtttctgaccgggactctggggtcccagacagattcagcggcagtgggtcaggcactgatttcacactgcaaatcagcagggtggaggctgaggatgttggcctttattactgcatgcaaggttcacactggccgctcactttcggcggagggaccaaggtggagatcaaac

SEQ ID NO: 397 - protein, 112 aa, Homo Sapiens
DVVMTQSPLSLPVTLGQPASISCRSSQSLVFSDGNTYLTWFQQRPGQSPRRLIYKVSDRDSGVPDRFSGSGSGTDFTLQISRVEAEDVGLYYCMQGSHWPLTFGGGTKVEIK

SEQ ID NO: 398 - protein, 5 aa, Homo Sapiens
TSAMT

SEQ ID NO: 399 - protein, 8 aa, Homo Sapiens
RFTFSTSA

SEQ ID NO: 400 - protein, 17 aa, Homo Sapiens
VISASGTTTYYGDSVKG

SEQ ID NO: 401 - protein, 8 aa, Homo Sapiens
ISASGTTT

SEQ ID NO: 402 - protein, 10 aa, Homo Sapiens
GGLAVPGPNY

SEQ ID NO: 403 - protein, 12 aa, Homo Sapiens
AKGGLAVPGPNY

SEQ ID NO: 404 - protein, 16 aa, Homo Sapiens
RSSQSLVFSDGNTYLT

SEQ ID NO: 405 - protein, 11 aa, Homo Sapiens
QSLVFSDGNTY

SEQ ID NO: 406 - protein, 7 aa, Homo Sapiens
KVSDRDS

SEQ ID NO: 407 - protein, 3 aa, Homo Sapiens
KVS

SEQ ID NO: 408 - protein, 9 aa, Homo Sapiens
MQGSHWPLT

SEQ ID NO: 409 - protein, 9 aa, Homo Sapiens
MQGSHWPLT

SEQ ID NO: 410 - DNA, 349 bp, Homo Sapiens
caggttcagttggtacagtctggacctgaagtgaagaagcctggggcctcagtgaaggtctcctgccaggcttctggttatacctttaccacctatggcatcaactgggtgcgacaggcccctggacaaggacttgagtggatgggaaggatcagcgcttacaatggtaacacaaattatgcacagaagttccagggcagagtcaccatgaccacagacacatctacgaggacagcctacatggagatgagtaacctgatatctgacgacacggccgtgtattattgtgcgcgagatggggctccccaagaccactggggccagggaaccctaatcaccgtctcttcag

SEQ ID NO: 411 - protein, 116 aa, Homo Sapiens
QVQLVQSGPEVKKPGASVKVSCQASGYTFTTYGINWVRQAPGQGLEWMGRISAYNGNTNYAQKFQGRVTMTTDTSTRTAYMEMSNLISDDTAVYYCARDGAPQDHWGQGTLITVSS

SEQ ID NO: 412 - DNA, 322 bp, Homo Sapiens
gacatccagatgacccagtctccatcctcactgtctgtatctgtaggagacagaatcaccatcacctgtcgggcgagtcaggacattagtaattctttagcctggtttcagcagaaaccagggaaagcccctaagtccctgatctttgctgcatccagtttgcaaagtggggtcccatcaaagttcagcggcagtggatctgggacagatttcaattttaccatcagcagcctgcagcctgaagattttgcaacttattactgccaacagtataatagtttccctctcactttcggcggagggaccaaggtggagatcaaac

SEQ ID NO: 413 - protein, 107 aa, Homo Sapiens
DIQMTQSPSSLSVSVGDRITITCRASQDISNSLAWFQQKPGKAPKSLIFAASSLQSGVPSKFSGSGSGTDFNFTISSLQPEDFATYYCQQYNSFPLTFGGGTKVEIK

SEQ ID NO: 414 - protein, 5 aa, Homo Sapiens
TYGIN

SEQ ID NO: 415 - protein, 8 aa, Homo Sapiens
GYTFTTYG

SEQ ID NO: 416 - protein, 17 aa, Homo Sapiens
RISAYNGNTNYAQKFQG

SEQ ID NO: 417 - protein, 8 aa, Homo Sapiens
ISAYNGNT

SEQ ID NO: 418 - protein, 7 aa, Homo Sapiens
DGAPQDH

SEQ ID NO: 419 - protein, 9 aa, Homo Sapiens
ARDGAPQDH

SEQ ID NO: 420 - protein, 11 aa, Homo Sapiens
RASQDISNSLA

SEQ ID NO: 421 - protein, 6 aa, Homo Sapiens
QDISNS

SEQ ID NO: 422 - protein, 7 aa, Homo Sapiens
AASSLQS

SEQ ID NO: 423 - protein, 3 aa, Homo Sapiens
AAS

SEQ ID NO: 424 - protein, 9 aa, Homo Sapiens
QQYNSFPLT

SEQ ID NO: 425 - protein, 9 aa, Homo Sapiens
QQYNSFPLT

SEQ ID NO: 426 - DNA, 352 bp, Homo Sapiens
gaggtgcagctggtggagtctgggggaggcctggtcaagcctggggggtccctgagactctcctgtgcagcctctggattcaccttcagtacctatagcatgaactgggtccgccaggctccagggaaggggctggagtgggtctcatccattagtcgtagtagtagttacatatactacgcagactcagtgaagggccgattcaccatctccagagacaacgccaagaactcactgtctctgcaaatgaacagcctgagagccgaggacacagctgtgtattactgtgcgagaggagcagcagctggtacggactactggggccagggaaccctggtcaccgtctcctcag

SEQ ID NO: 427 - protein, 117 aa, Homo Sapiens
EVQLVESGGGLVKPGGSLRLSCAASGFTFSTYSMNWVRQAPGKGLEWVSSISRSSSYIYYADSVKGRFTISRDNAKNSLSLQMNSLRAEDTAVYYCARGAAAGTDYWGQGTLVTVSS

SEQ ID NO: 428 - DNA, 337 bp, Homo Sapiens
gatattgtgatgacccagactccactctcctcacctgtcacccttggacagccggcctccatctcctgcaggtctagtcaaagcctcgtacacagtgatggaaacacctacttgagttggcttcagcagaggccaggccagcctccaagactcctaatttataggatttctaaccggttctctggggtcccagacagattcagtggcagtggggcagggacagatttcacactgaaaatcagcagggtggaagctgaggatgtcggggtttattactgcatgcaaactacacaatttccgctcactttcggcggagggaccaaggtggagatcaaac

SEQ ID NO: 429 - protein, 112 aa, Homo Sapiens
DIVMTQTPLSSPVTLGQPASISCRSSQSLVHSDGNTYLSWLQQRPGQPPRLLIYRISNRFSGVPDRFSGSGAGTDFTLKISRVEAEDVGVYYCMQTTQFPLTFGGGTKVEIK

SEQ ID NO: 430 - protein, 5 aa, Homo Sapiens
TYSMN

SEQ ID NO: 431 - protein, 8 aa, Homo Sapiens
GFTFSTYS

SEQ ID NO: 432 - protein, 17 aa, Homo Sapiens
SISRSSSYIYYADSVKG

SEQ ID NO: 433 - protein, 8 aa, Homo Sapiens
ISRSSSYI

SEQ ID NO: 434 - protein, 8 aa, Homo Sapiens
GAAAGTDY

SEQ ID NO: 435 - protein, 10 aa, Homo Sapiens
ARGAAAGTDY

SEQ ID NO: 436 - protein, 16 aa, Homo Sapiens
RSSQSLVHSDGNTYLS

SEQ ID NO: 437 - protein, 11 aa, Homo Sapiens
QSLVHSDGNTY

SEQ ID NO: 438 - protein, 7 aa, Homo Sapiens
RISNRFS

SEQ ID NO: 439 - protein, 3 aa, Homo Sapiens
RIS

SEQ ID NO: 440 - protein, 9 aa, Homo Sapiens
MQTTQFPLT

SEQ ID NO: 441 - protein, 9 aa, Homo Sapiens
MQTTQFPLT

SEQ ID NO: 442 - DNA, 376 bp, Homo Sapiens
caggtccagcttgtgcagtctggggctgaggtgaggaagcctggggcctcagtgaaggtttcctgcaaggcttctggatacaccttcactgactatgcactacattgggtgcgccaggcccccggacaaaggcttgagtggatgggatggatcatcgttggcaatggtaagacaagatatgcacagaagttccagggcagagtcaccattacctgggaaacatccgcgagcacagccttcgtggacctgaatagtctgagatcggaagacacggctgtttattactgtgcgagagggtttactatggttcggggagccccctattatgacggtttggacgtctggggccaagggaccacggtcaccgtctcctcag

SEQ ID NO: 443 - protein, 125 aa, Homo Sapiens
QVQLVQSGAEVRKPGASVKVSCKASGYTFTDYALHWVRQAPGQRLEWMGWIIVGNGKTRYAQKFQGRVTITWETSASTAFVDLNSLRSEDTAVYYCARGFTMVRGAPYYDGLDVWGQGTTVTVSS

SEQ ID NO: 444 - DNA, 334 bp, Homo Sapiens
gatattgtgatgactcagtctccactctccctgtccgtcacccctggagagccggcctccatctcctgcaggtctagtcagagcctcctgtatagtactggatacaactatttggattggtacctgcagaagccagggctgtctccacagctcctgatctatttgggttctaatcgggcctccggggtccctgacaggttcagtggcagtggatcaggcacagattttacactgaagatcagcagagtggaggctgaagatgttgggttttattactgcatgcaagctctacaagctcccactttcggcggagggaccaaggtggagatcaaac

SEQ ID NO: 445 - protein, 111 aa, Homo Sapiens
DIVMTQSPLSLSVTPGEPASISCRSSQSLLYSTGYNYLDWYLQKPGLSPQLLIYLGSNRASGVPDRFSGSGSGTDFTLKISRVEAEDVGFYYCMQALQAPTFGGGTKVEIK

SEQ ID NO: 446 - protein, 5 aa, Homo Sapiens
DYALH

SEQ ID NO: 447 - protein, 8 aa, Homo Sapiens
GYTFTDYA

SEQ ID NO: 448 - protein, 17 aa, Homo Sapiens
WIIVGNGKTRYAQKFQG

SEQ ID NO: 449 - protein, 8 aa, Homo Sapiens
IIVGNGKT

SEQ ID NO: 450 - protein, 16 aa, Homo Sapiens
GFTMVRGAPYYDGLDV

SEQ ID NO: 451 - protein, 18 aa, Homo Sapiens
ARGFTMVRGAPYYDGLDV

SEQ ID NO: 452 - protein, 16 aa, Homo Sapiens
RSSQSLLYSTGYNYLD

SEQ ID NO: 453 - protein, 11 aa, Homo Sapiens
QSLLYSTGYNY

SEQ ID NO: 454 - protein, 7 aa, Homo Sapiens
LGSNRAS

SEQ ID NO: 455 - protein, 3 aa, Homo Sapiens
LGS

SEQ ID NO: 456 - protein, 8 aa, Homo Sapiens
MQALQAPT

SEQ ID NO: 457 - protein, 8 aa, Homo Sapiens
MQALQAPT

SEQ ID NO: 458 - DNA, 376 bp, Homo Sapiens
caggtccaacttgtgcagtctggggctgaggtgaggaagcctggggcctcagtgaaggtttcctgcaaggcttctggatacaccttcactgactatgcaatacattgggtgcgccaggcccccggacaaaggcttgagtggatgggatggatcatcgttggcaatggtaagacaagatatgcacagaagttccagggcagagtcaccattacctgggaaacatccgcgagcacagcctacatggacctgagtagtctgagatcggaagacacggctgtttattactgtgcgagagggtttactatggttcggggagccccctattatgacggtttggacgtctggggccaggggaccacggtcaccgtctcctcag

SEQ ID NO: 459 - protein, 125 aa, Homo Sapiens
QVQLVQSGAEVRKPGASVKVSCKASGYTFTDYAIHWVRQAPGQRLEWMGWIIVGNGKTRYAQKFQGRVTITWETSASTAYMDLSSLRSEDTAVYYCARGFTMVRGAPYYDGLDVWGQGTTVTVSS

SEQ ID NO: 460 - DNA, 334 bp, Homo Sapiens
gatattgtgatgactcagtctccactctccctgcccgtcacccctggagagccggcctccatctcctgcaggtctagtcagagcctcctgtttagtactggatacaactatttggattggtacctgcagaagccagggctgtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcccactttcggcggagggaccaaggtggagatcaaac

SEQ ID NO: 461 - protein, 111 aa, Homo Sapiens
DIVMTQSPLSLPVTPGEPASISCRSSQSLLFSTGYNYLDWYLQKPGLSPQLLIYLGSNRASGVPDRFSGSGSGTDFTLKISRVEAEDVGVYYCMQALQTPTFGGGTKVEIK

SEQ ID NO: 462 - protein, 5 aa, Homo Sapiens
DYAIH

SEQ ID NO: 463 - protein, 8 aa, Homo Sapiens
GYTFTDYA

SEQ ID NO: 464 - protein, 17 aa, Homo Sapiens
WIIVGNGKTRYAQKFQG

SEQ ID NO: 465 - protein, 8 aa, Homo Sapiens
IIVGNGKT

SEQ ID NO: 466 - protein, 16 aa, Homo Sapiens
GFTMVRGAPYYDGLDV

SEQ ID NO: 467 - protein, 18 aa, Homo Sapiens
ARGFTMVRGAPYYDGLDV

SEQ ID NO: 468 - protein, 16 aa, Homo Sapiens
RSSQSLLFSTGYNYLD

SEQ ID NO: 469 - protein, 11 aa, Homo Sapiens
QSLLFSTGYNY

SEQ ID NO: 470 - protein, 7 aa, Homo Sapiens
LGSNRAS

SEQ ID NO: 471 - protein, 3 aa, Homo Sapiens
LGS

SEQ ID NO: 472 - protein, 8 aa, Homo Sapiens
MQALQTPT

SEQ ID NO: 473 - protein, 8 aa, Homo Sapiens
MQALQTPT

SEQ ID NO: 474 - DNA, 382 bp, Homo Sapiens
caggtgcagctgcaggagtcgggcccaggactggtgaagccttcggggaccctgtccctcacttgcgctgtctctggtggctccatcagtagtagtaactggtggagttgggtccgccagcccccagggaaggggctggagtggattggggaaatctatcatagtgggaacaccaactacaacccgtccctcaagagtcgagtcaccatatcagtagacaagtccaagaaccagttctccctgaagctgagctctgtgaccgccgcggacacggccgtgtattactgtgtgagaggttattactatgattcggggacctcttgggggtactattatggtatggacgtctggggccaagggaccacggtcaccgtctcctcag

SEQ ID NO: 475 - protein, 127 aa, Homo Sapiens
QVQLQESGPGLVKPSGTLSLTCAVSGGSISSSNWWSWVRQPPGKGLEWIGEIYHSGNTNYNPSLKSRVTISVDKSKNQFSLKLSSVTAADTAVYYCVRGYYYDSGTSWGYYYGMDVWGQGTTVTVSS

SEQ ID NO: 476 - DNA, 322 bp, Homo Sapiens
gacatccagatgacccagtctccatcctcactgtctgcatctgtaggagacagagtcaccatcacttgtcgggcgagtcagggcattagcaattatttagcctggtttcagcagagaccagggagagcccctaagtcccttatctatgctgcatccagtttgcaaaggggggtcccatcaaagttcagcggcagtggatctgggacagatttcactctcaccatcagcagcctgcagcctgaagattttgcaacttattactgccaacagtataatagttacccgatcaccttcggccaagggacacgactggagatcaaac

SEQ ID NO: 477 - protein, 107 aa, Homo Sapiens
DIQMTQSPSSLSASVGDRVTITCRASQGISNYLAWFQQRPGRAPKSLIYAASSLQRGVPSKFSGSGSGTDFTLTISSLQPEDFATYYCQQYNSYPITFGQGTRLEIK

SEQ ID NO: 478 - protein, 6 aa, Homo Sapiens
SSNWWS

SEQ ID NO: 479 - protein, 9 aa, Homo Sapiens
GGSISSSNW

SEQ ID NO: 480 - protein, 16 aa, Homo Sapiens
EIYHSGNTNYNPSLKS

SEQ ID NO: 481 - protein, 7 aa, Homo Sapiens
IYHSGNT

SEQ ID NO: 482 - protein, 18 aa, Homo Sapiens
GYYYDSGTSWGYYYGMDV

SEQ ID NO: 483 - protein, 20 aa, Homo Sapiens
VRGYYYDSGTSWGYYYGMDV

SEQ ID NO: 484 - protein, 11 aa, Homo Sapiens
RASQGISNYLA

SEQ ID NO: 485 - protein, 6 aa, Homo Sapiens
QGISNY

SEQ ID NO: 486 - protein, 7 aa, Homo Sapiens
AASSLQR

SEQ ID NO: 487 - protein, 3 aa, Homo Sapiens
AAS

SEQ ID NO: 488 - protein, 9 aa, Homo Sapiens
QQYNSYPIT

SEQ ID NO: 489 - protein, 9 aa, Homo Sapiens
QQYNSYPIT

SEQ ID NO: 490 - DNA, 376 bp, Homo Sapiens
caggtccagcttgtgcagtctgggcctgaggtgaggaaccctggggcctcagtgagggtttcctgcaaggcttctggatacaccttcactgactatgcactacattgggtgcgccaggcccccggacaaaggcttgagtggatgggatggatcatcgttggcaatggtaagacaagatatgcacagaagttccagggcagagtcaccattacctgggaaacatccgcgagcacagccttcatggacctgagtagtctgagatcggaagacacggctgtttattactgtgcgagagggtttactatggttcggggagccccctattatgacggtttggacgtctggggccaagggaccacggtcaccatctcctcag

SEQ ID NO: 491 - protein, 125 aa, Homo Sapiens
QVQLVQSGPEVRNPGASVRVSCKASGYTFTDYALHWVRQAPGQRLEWMGWIIVGNGKTRYAQKFQGRVTITWETSASTAFMDLSSLRSEDTAVYYCARGFTMVRGAPYYDGLDVWGQGTTVTISS

SEQ ID NO: 492 - DNA, 337 bp, Homo Sapiens
gatattgtaatgactcagtctccactctccctgcccgtcacccttggacagccggcctccatctcctgcaggtctagtcaaagcctcatatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaagatgttggagtttattactgcttgcaaggtactctctggccgatcaccttcggccaagggacacgactggagatcaaac

SEQ ID NO: 493 - protein, 112 aa, Homo Sapiens
DIVMTQSPLSLPVTLGQPASISCRSSQSLIYSDGNTYLNWFQQRPGQSPRRLIYKVSNRDSGVPDRFSGSGSGTDFTLKISRVEAEDVGVYYCLQGTLWPITFGQGTRLEIK

SEQ ID NO: 494 - protein, 5 aa, Homo Sapiens
DYALH

SEQ ID NO: 495 - protein, 8 aa, Homo Sapiens
GYTFTDYA

SEQ ID NO: 496 - protein, 17 aa, Homo Sapiens
WIIVGNGKTRYAQKFQG

SEQ ID NO: 497 - protein, 8 aa, Homo Sapiens
IIVGNGKT

SEQ ID NO: 498 - protein, 16 aa, Homo Sapiens
GFTMVRGAPYYDGLDV

SEQ ID NO: 499 - protein, 18 aa, Homo Sapiens
ARGFTMVRGAPYYDGLDV

SEQ ID NO: 500 - protein, 16 aa, Homo Sapiens
RSSQSLIYSDGNTYLN

SEQ ID NO: 501 - protein, 11 aa, Homo Sapiens
QSLIYSDGNTY

SEQ ID NO: 502 - protein, 7 aa, Homo Sapiens
KVSNRDS

SEQ ID NO: 503 - protein, 3 aa, Homo Sapiens
KVS

SEQ ID NO: 504 - protein, 9 aa, Homo Sapiens
LQGTLWPIT

SEQ ID NO: 505 - protein, 9 aa, Homo Sapiens
LQGTLWPIT

SEQ ID NO: 506 - DNA, 376 bp, Homo Sapiens
caggtccagtttgtgcagtctggggctgaggtgaggaagcctggggcctcagtgaaagtttcctgcaaggcttctggatacaccttcactgactatgcaatacattgggtgcgccaggcccccggacaaaggcttgagtggatgggatggatcatcgttggcaatggtaagacaagatattcacagaagtttcagggcagactcaccattacctgggaaacatccgcgaccacagcctacatggacctgagtagtctgagatcggaggacacggctgtttattactgtgcgagagggtttactatggttcggggagccccctattatgacggtatggacgtctggggccaagggaccacggtcaccgtctcctcag

SEQ ID NO: 507 - protein, 125 aa, Homo Sapiens
QVQFVQSGAEVRKPGASVKVSCKASGYTFTDYAIHWVRQAPGQRLEWMGWIIVGNGKTRYSQKFQGRLTITWETSATTAYMDLSSLRSEDTAVYYCARGFTMVRGAPYYDGMDVWGQGTTVTVSS

SEQ ID NO: 508 - DNA, 334 bp, Homo Sapiens; identical to SEQ ID NO: 364

SEQ ID NO: 509 - protein, 111 aa, Homo Sapiens; identical to SEQ ID NO: 365

SEQ ID NO: 510 - protein, 5 aa, Homo Sapiens
DYAIH

SEQ ID NO: 511 - protein, 8 aa, Homo Sapiens
GYTFTDYA

SEQ ID NO: 512 - protein, 17 aa, Homo Sapiens
WIIVGNGKTRYSQKFQG

SEQ ID NO: 513 - protein, 8 aa, Homo Sapiens
IIVGNGKT

SEQ ID NO: 514 - protein, 16 aa, Homo Sapiens
GFTMVRGAPYYDGMDV

SEQ ID NO: 515 - protein, 18 aa, Homo Sapiens
ARGFTMVRGAPYYDGMDV

SEQ ID NO: 516 - protein, 16 aa, Homo Sapiens
RSSQSLLYTTGYNYLD

SEQ ID NO: 517 - protein, 11 aa, Homo Sapiens
QSLLYTTGYNY

SEQ ID NO: 518 - protein, 7 aa, Homo Sapiens
LGSYRAS

SEQ ID NO: 519 - protein, 3 aa, Homo Sapiens
LGS

SEQ ID NO: 520 - protein, 8 aa, Homo Sapiens
MQALQAPT

SEQ ID NO: 521 - protein, 8 aa, Homo Sapiens
MQALQAPT

SEQ ID NO: 522 - DNA, 376 bp, Homo Sapiens; identical to SEQ ID NO: 490

SEQ ID NO: 523 - protein, 125 aa, Homo Sapiens; identical to SEQ ID NO: 491

SEQ ID NO: 524 - DNA, 334 bp, Homo Sapiens
gatattgtgatgactcagtctccactctccctgcccgtcagccctggagagccggcctccatctcctgcaggtctagtcagagcctcctgtatagcactggatacaactatttggattggtacctgcagaagccagggctgtctcctcagctcctgatctatttgggttctattcgggcctccggggtccctgacaggttcagtggcagtggatcaggcacagattttacactgagcatcagcagagtggaggctgaggatgttggaatttattactgcatgcaagctctacaaactcccactttcggcggagggaccaaggtggagatcaaac

SEQ ID NO: 525 - protein, 111 aa, Homo Sapiens
DIVMTQSPLSLPVSPGEPASISCRSSQSLLYSTGYNYLDWYLQKPGLSPQLLIYLGSIRASGVPDRFSGSGSGTDFTLSISRVEAEDVGIYYCMQALQTPTFGGGTKVEIK

SEQ ID NO: 526 - protein, 5 aa, Homo Sapiens
DYALH

SEQ ID NO: 527 - protein, 8 aa, Homo Sapiens
GYTFTDYA

SEQ ID NO: 528 - protein, 17 aa, Homo Sapiens
WIIVGNGKTRYAQKFQG

SEQ ID NO: 529 - protein, 8 aa, Homo Sapiens
IIVGNGKT

SEQ ID NO: 530 - protein, 16 aa, Homo Sapiens
GFTMVRGAPYYDGLDV

SEQ ID NO: 531 - protein, 18 aa, Homo Sapiens
ARGFTMVRGAPYYDGLDV

SEQ ID NO: 532 - protein, 16 aa, Homo Sapiens
RSSQSLLYSTGYNYLD

SEQ ID NO: 533 - protein, 11 aa, Homo Sapiens
QSLLYSTGYNY

SEQ ID NO: 534 - protein, 7 aa, Homo Sapiens
LGSIRAS

SEQ ID NO: 535 - protein, 3 aa, Homo Sapiens
LGS

SEQ ID NO: 536 - protein, 8 aa, Homo Sapiens
MQALQTPT

SEQ ID NO: 537 - protein, 8 aa, Homo Sapiens
MQALQTPT

SEQ ID NO: 538 - DNA, 376 bp, Homo Sapiens
caggtccagcttgtgcagtctggggctgaggtgaggaagcctggggcctcagtgaaggtttcctgcaaggcttctggatacaccttcactgactatgcaatacattgggtgcgccaggcccccggacaaaggcttgagtggatgggatggatcatcgttggcaatggtaagacaagatattcacagaagtttcagggcagagtcaccattacctgggaaacatccgcgagcacagcctacatggacctgagtagtctgagatcggaagacacggctgtttattactgtgcgagagggtttactatggttcggggagccccctattatgacggtatggacgtctggggccaagggaccacggtcaccgtctcctcag

SEQ ID NO: 539 - protein, 125 aa, Homo Sapiens; identical to SEQ ID NO: 331

SEQ ID NO: 540 - DNA, 337 bp, Homo Sapiens
gatgttgtaatgactcagtctccactctccctgcccgtcacccttggacagccggcctccatctcctgcaggtctagtcaaagcctcgtatacagtgatggaaacacctacttgaattggtttcagcagaggccaggccaatctccacggcgcctaatttataaggtttctaaccgggactctggggtcccagacagattcagcggcagtgggtcaggcactgatttcacactgaaaatcagcagggtggaggctgaagatgttggagtttattactgcatgcaaggtactctctggccgatcaccctcggccaagggacacgactggagatcaaac

SEQ ID NO: 541 - protein, 112 aa, Homo Sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTLWPITLGQGTRLEIK

SEQ ID NO: 542 - protein, 5 aa, Homo Sapiens
DYAIH

SEQ ID NO: 543 - protein, 8 aa, Homo Sapiens
GYTFTDYA

SEQ ID NO: 544 - protein, 17 aa, Homo Sapiens
WIIVGNGKTRYSQKFQG

SEQ ID NO: 545 - protein, 8 aa, Homo Sapiens
IIVGNGKT

SEQ ID NO: 546 - protein, 16 aa, Homo Sapiens
GFTMVRGAPYYDGMDV

SEQ ID NO: 547 - protein, 18 aa, Homo Sapiens
ARGFTMVRGAPYYDGMDV

SEQ ID NO: 548 - protein, 16 aa, Homo Sapiens
RSSQSLVYSDGNTYLN

SEQ ID NO: 549 - protein, 11 aa, Homo Sapiens
QSLVYSDGNTY

SEQ ID NO: 550 - protein, 7 aa, Homo Sapiens
KVSNRDS

SEQ ID NO: 551 - protein, 3 aa, Homo Sapiens
KVS

SEQ ID NO: 552 - protein, 9 aa, Homo Sapiens
MQGTLWPIT

SEQ ID NO: 553 - protein, 9 aa, Homo Sapiens
MQGTLWPIT

SEQ ID NO: 554 - DNA, 376 bp, Homo Sapiens
caggtccagcttgtgcagtctggggctgaggtgaggaagcctggggcctcagtgaaggtttcctgcaaggcttctggatacaccttcactgactatgcactacattgggtgcgccaggcccccggacaaaggcttgagtggatgggatggatcatcgttggcaatggtaagacaagatatgcacagaagttccagggcagagtcaccattacctgggaaacatccgcgagcacagccttcatggacctgagtagtctgagatcggaagacacggctgtttattactgtgcgagagggtttactatggttcggggagccccctattatgacggtttggacgtctggggccaagggaccacggtcaccgtctcctcag

SEQ ID NO: 555 - protein, 125 aa, Homo Sapiens
QVQLVQSGAEVRKPGASVKVSCKASGYTFTDYALHWVRQAPGQRLEWMGWIIVGNGKTRYAQKFQGRVTITWETSASTAFMDLSSLRSEDTAVYYCARGFTMVRGAPYYDGLDVWGQGTTVTVSS

SEQ ID NO: 556 - DNA, 334 bp, Homo Sapiens
gatattgtgatgactcagtctccactctccctgcccgtcacccctggagagccggcctccatctcctgcaggtctagtcagagcctcctgtatagtactggatacaactatttggattggtacctgcagaagccagggctgtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcccactttcggcggagggaccaaggtggagatcaaac

SEQ ID NO: 557 - protein, 111 aa, Homo Sapiens; identical to SEQ ID NO: 333

SEQ ID NO: 558 - protein, 5 aa, Homo Sapiens
DYALH

SEQ ID NO: 559 - protein, 8 aa, Homo Sapiens
GYTFTDYA

SEQ ID NO: 560 - protein, 17 aa, Homo Sapiens
WIIVGNGKTRYAQKFQG

SEQ ID NO: 561 - protein, 8 aa, Homo Sapiens
IIVGNGKT

SEQ ID NO: 562 - protein, 16 aa, Homo Sapiens
GFTMVRGAPYYDGLDV

SEQ ID NO: 563 - protein, 18 aa, Homo Sapiens
ARGFTMVRGAPYYDGLDV

SEQ ID NO: 564 - protein, 16 aa, Homo Sapiens
RSSQSLLYSTGYNYLD

SEQ ID NO: 565 - protein, 11 aa, Homo Sapiens
QSLLYSTGYNY

SEQ ID NO: 566 - protein, 7 aa, Homo Sapiens
LGSNRAS

SEQ ID NO: 567 - protein, 3 aa, Homo Sapiens
LGS

SEQ ID NO: 568 - protein, 8 aa, Homo Sapiens
MQALQTPT

SEQ ID NO: 569 - protein, 8 aa, Homo Sapiens
MQALQTPT

SEQ ID NO: 570 - DNA, 352 bp, Homo Sapiens; identical to SEQ ID NO: 426

SEQ ID NO: 571 - protein, 117 aa, Homo Sapiens; identical to SEQ ID NO: 427

SEQ ID NO: 572 - DNA, 337 bp, Homo Sapiens; identical to SEQ ID NO: 492

SEQ ID NO: 573 - protein, 112 aa, Homo Sapiens; identical to SEQ ID NO: 493

SEQ ID NO: 574 - protein, 5 aa, Homo Sapiens
TYSMN

SEQ ID NO: 575 - protein, 8 aa, Homo Sapiens
GFTFSTYS

SEQ ID NO: 576 - protein, 17 aa, Homo Sapiens
SISRSSSYIYYADSVKG

SEQ ID NO: 577 - protein, 8 aa, Homo Sapiens
ISRSSSYI

SEQ ID NO: 578 - protein, 8 aa, Homo Sapiens
GAAAGTDY

SEQ ID NO: 579 - protein, 10 aa, Homo Sapiens
ARGAAAGTDY

SEQ ID NO: 580 - protein, 16 aa, Homo Sapiens
RSSQSLIYSDGNTYLN

SEQ ID NO: 581 - protein, 11 aa, Homo Sapiens
QSLIYSDGNTY

SEQ ID NO: 582 - protein, 7 aa, Homo Sapiens
KVSNRDS

SEQ ID NO: 583 - protein, 3 aa, Homo Sapiens
KVS

SEQ ID NO: 584 - protein, 9 aa, Homo Sapiens
LQGTLWPIT

SEQ ID NO: 585 - protein, 9 aa, Homo Sapiens
LQGTLWPIT

SEQ ID NO: 586 - DNA, 376 bp, Homo Sapiens
caggtccagcttgtgcagtctggggctgaggtgaggaagcctggggcctcagtgaaggtttcctgcaaggcttctggatacaccttcactgactatgcaatacattgggtgcgccaggcccccggacaaaggcttgagtggatgggatggatcatcgttggcaatggtaagacaagatatgcacagaagttgcagggcagagtcaccattacctgggaaacatccgcgagcacagcctacatggacctgactagtctgagatcggaagacacggctgtttattactgtgcgagagggtttactatggttcggggagccccctattatgacggtttggacgtctggggccaagggaccacggtcaccgtctcctcag

SEQ ID NO: 587 - protein, 125 aa, Homo Sapiens
QVQLVQSGAEVRKPGASVKVSCKASGYTFTDYAIHWVRQAPGQRLEWMGWIIVGNGKTRYAQKLQGRVTITWETSASTAYMDLTSLRSEDTAVYYCARGFTMVRGAPYYDGLDVWGQGTTVTVSS

SEQ ID NO: 588 - DNA, 334 bp, Homo Sapiens
gatattgtgatgactcagtctccactctccctgcccgtcacccctggagagccggcctccatctcctgcaggtctagtcagagcctcctgtatagtactggatacaactatttggattggtacctgcagaagccagggctgtctccacatctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcccactttcggcggagggaccaaggtggagatcaaac

SEQ ID NO: 589 - protein, 111 aa, Homo Sapiens
DIVMTQSPLSLPVTPGEPASISCRSSQSLLYSTGYNYLDWYLQKPGLSPHLLIYLGSNRASGVPDRFSGSGSGTDFTLKISRVEAEDVGVYYCMQALQTPTFGGGTKVEIK

SEQ ID NO: 590 - protein, 5 aa, Homo Sapiens
DYAIH

SEQ ID NO: 591 - protein, 8 aa, Homo Sapiens
GYTFTDYA

SEQ ID NO: 592 - protein, 17 aa, Homo Sapiens
WIIVGNGKTRYAQKLQG

SEQ ID NO: 593 - protein, 8 aa, Homo Sapiens
IIVGNGKT

SEQ ID NO: 594 - protein, 16 aa, Homo Sapiens
GFTMVRGAPYYDGLDV

SEQ ID NO: 595 - protein, 18 aa, Homo Sapiens
ARGFTMVRGAPYYDGLDV

SEQ ID NO: 596 - protein, 16 aa, Homo Sapiens
RSSQSLLYSTGYNYLD

SEQ ID NO: 597 - protein, 11 aa, Homo Sapiens
QSLLYSTGYNY

SEQ ID NO: 598 - protein, 7 aa, Homo Sapiens
LGSNRAS

SEQ ID NO: 599 - protein, 3 aa, Homo Sapiens
LGS

SEQ ID NO: 600 - protein, 8 aa, Homo Sapiens
MQALQTPT

SEQ ID NO: 601 - protein, 8 aa, Homo Sapiens
MQALQTPT

SEQ ID NO: 602 - protein, 327 aa, Artificial Sequence
  IgG4 heavy chain constant region IgG4-PE
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2017/0306013 A1
(43) Pub. Date: Oct. 26, 2017

(54) SELECTIVE NAV PROTEIN BINDERS

(71) Applicant: Kymab Limited, Cambridge (GB)

(72) Inventors: Nigel Clark, Cambridge (GB); E-Chiang Lee, Cambridge (GB)

(73) Assignee: Kymab Limited, Cambridge (GB)

(21) Appl. No.: 15/520,204

(22) PCT Filed: Oct. 20, 2015
(86) PCT No.: PCT/GB2015/053110

§ 371 Date: Apr. 19, 2017

(30) Foreign Application Priority Data
Oct. 21, 2014 (GB) 1418713.2

(51) Int. Cl. C07K 16/28; A61K 39/00

(52) U.S. Cl. CPC C07K 16/28; A61K 2039/505

(57) ABSTRACT

The invention provides antibodies and fragments thereof which bind to human Nav1.7, as well as nucleic acids, vectors, host cells and hybridomas for making the same. Further provided are pharmaceutical compositions for use in treating, preventing and/or reducing the risk of a NAV1.7-mediated conditions or diseases, such as pain, inflammation, and metabolic/chronic diseases. The invention additionally provides methods of generating an antibody against a NAV protein of interest.

The present invention relates to antibodies and fragments thereof which are selective for human NAV1.7 and/or 1.8 and/or 1.9, their uses and methods of treatment of pain and other such diseases.

### BACKGROUND OF THE INVENTION

Voltage-gated sodium channels (VGSCs or Naᵥs) are a class of ion channel that are activated by changes of electrical membrane potential near the channel and thereby help to establish and control cell membrane potential of cells by allowing the flow of ions down their electrochemical gradient. These types of channel are especially critical in neurons and muscles. The human genome contains more than 400 ion channel genes presenting a large diversity and play critical roles in many cellular processes such as secretion, muscular contraction and the generation and propagation of action potentials in cardiac and neuronal tissues. In mammals, we know of 9 isoforms of the sodium-channel a subunit (Nav1.1-Nav1.9), each with a unique central and peripheral nervous system distribution. Four closely related sodium channels (Nav1.1, -1.2, -1.3, and -1.7) are encoded by a set of 4 genes (SCN1A, SCN2A, SCN3A, and SCN9A, respectively) located within a cluster on chromosome 2q24.3. They play an important role in excitable neuronal and muscle cells, driving a rapid and coordinated depolarization and polarization in response to triggering voltage change. For example, expressed along the sensory terminal, the axon and the synapse, NAV1.7 directionally propagates electrical signals essential for pain.

VGSCs are integral membrane proteins, which share a conserved architecture. They are complexes consisting of a large central pore-forming α-subunit and two smaller auxiliary β-subunits. The pore-forming α-subunit is sufficient for α functional expression, but the kinetics and voltage dependence of channel gating are modified by the β-subunits. The α-subunits are composed of four homologous domains (D1-D4), each having six transmembrane regions, designated S1-S6. The opening and closing of the ion channel pore, referred as the gating process, may be triggered by various cellular or biochemical processes. The S4 regions or voltage sensors contain the gating charge arginine residues that sense membrane potential changes and control the motion of the gate for pore opening, closing and inactivation.

There are ten cloned α-subunits and four β-subunits. These distinct sodium channels have similar structural and functional properties, but they initiate action potential in different cell types and have distinct regulatory and pharmacological properties. The ten different genes encode ten isoforms of the sodium channel protein, and while they all share a common structure, they have different amino acid sequences.

Nav1.7 is encoded by SCN9A, and is important for electrical signaling primarily in nociceptive dorsal root ganglia neurons and sympathetic ganglion neurons. It is expressed at the endings of nociceptors close to where the impulse is initiated (Toledo-Aral, et al. (1997), PNAS 94:1527-1532). The Nav1.5 and Nav1.4 channels are the major sodium channel isoforms expressed in the cardiac and muscular tissue, respectively whereas Nav1.1, 1.2, 1.3, 1.6, 1.7, 1.8 and 1.9 are specifically expressed in the central and peripheral nervous system. The use of the natural occurring toxin, tetrodotoxin (TTX), allowed to establish a pharmacological classification of the sodium channel isoforms based on their affinity to the toxin. The voltage-gated sodium channels were thus classified as TTX resistant (Nav1.5, 1.8, 1.9) and TTX sensitive.

Although the architecture of Kv (potassium channel) has been established at high resolution of structure analysis, the structure basis for rapid, voltage-dependent activation of VGSCs was uncertain till the paper published by Payandeh et al. in Nature in 2011. They reported the crystal structure of a voltage sodium channel from Arobacter butzleri (NavAb) captured in a closed-pore conformation with four activated sensors at 2.7A resolution.

Scorpion α toxins and sea anemone toxins bind to the D4 S3-S4 (E2) loop and slow the coupling of sodium channel activation to inactivation. Scorpion β toxins, spider β toxins and μO-conotoxins bind to the D2 S3-S4 (E2) loop and slow down the activation of sodium channels. The binding of these neuropeptides are not specific due to the similarity of the binding regions among those VGSCs.

With systematic administration of the aforementioned neuropeptides to mice, no analgesic effect is observed, unless the C fiber neurons are desheathed, which suggests that the toxin cannot access expressed Nav1.7 on the intact neurons.

NAV1.7, 1.8 and 1.9 are believed to have the ability to modulate pain, such as neuropathic pain.

Nav1.7 is predominantly expressed in the dorsal root ganglion (DRG) neurons and sympathetic ganglion neurons (FIG. 1B, Drenth & Waxman). Immunohistochemical studies show that Nav1.7 is present at the distal ends of the wire-like projections of neurons known as neurites, close to the impulse trigger zone where neuronal firing is initiated. Interestingly, the large majority of DRG neurons that express Nav1.7 are pain sensing (nociceptive), suggesting a role for this sodium channel in the pathogenesis of pain. Both gain-of-function and loss-of-function mutations of Nav1.7 result in clear pain related abnormalities in humans. Erythromelalgia, an inherited neuropathy wherein patients experience a severe burning pain in response to mild warmth, appears to be the result of mutations in Nav 1.7, which cause excessive channel activity (Drenth et al. (2001), Am. 3. Hum. Genet. 68:1277-1282; Cummins et al., (2004), J. Neurosci 24:8232-8236). SCN9A mutations that resulted in the loss of Nav1.7 function, and which also resulted in the loss of pain, were identified in three families from Pakistan. All of the mutations observed were nonsense mutations with the majority of affected patients having homozygous mutations in the SCN9A gene. This observation linked the loss of Nav1.7 function with an inability to experience pain (Cox et al., (2006), Nature 444:894-898). From KO studies (Nassar et al. (2004), PNAS 101:12706-12711) and animal pain models, NAV1.7 plays a significant role in inflammatory pain.

Neuropathic pain is a highly prevalent condition. In the United States, it is estimated to affect between 0.6 and 1.5% of the population, or 1.8 to 4.5 million people (Pullar and Palmer, 2003, Drug News Perspect 16: 622-630). At least 1.4 million people each year are diagnosed with painful diabetic neuropathy (PDN), post-herpetic neuropathy (PHN) or trigeminal neuralgia (TN); three major causes of neuropathic pain. Other causes of neuropathic pain include spinal cord injuries, multiple sclerosis, phantom limb pain, post-stroke pain and HIV-associated pain. If patients with neuropathic-related chronic back pain, osteoarthritis and cancer were included, the total number would at least double. Nonsteroidal anti-inflammatory drugs (NSAIDs) although frequently prescribed, are not hugely effective in the treatment of neuropathic pain. Moreover, their chronic use may lead to serious gastric damage. On the other hand, the use of opioids (morphine and derivatives) is restricted to the most severe form of neuropathic pain, i.e., cancer-related neuropathy, because serious side-effects are associated with chronic treatment, such as nausea, emesis, respiratory depression, constipation and tolerance, and the potential for addiction and abuse. The latter have prevented the use of opioids in other neuropathies (Dellemijn, 1999, Pain, 80:453-462; Namaka et al., 2004, Clin Ther, 26:951-979). Anti-epileptic drugs (AEDs) are known to attenuate abnormal neural hyperexcitability in the brain. In view of neural hyperexcitability playing a crucial role in neuropathic pain, it is understandable that AEDs were aimed at the treatment of chronic neuropathic pain (Renfrey, Downton and Featherstone, 2003, Nat Rev Drug Discov, 2: 175-176). The most recent and important examples are gabapentin (Neurontin) and pregabalin (Lyrica, Frampton and Scott, 2004, Drugs, 64: 2813-2820). However, even gabapentin, the gold standard for the treatment of neuropathic pain, reduces pain at best by 50% in about 40% of patients (Dworkin, 2002, Clin J Pain, 18: 343-349). Further, in contrast to opioids, gabapentin is not used in the treatment of cancer-related neuropathic pain.

Lee et al. describes an antibody, designated SVmab1, generated by immunisation of mice with a linear peptide, which was shown to reduce inflammatory and neuropathic pain in a mouse model (Lee et al. (2014), Cell, 157:1-12).

Antibodies which bind to NAV1.7 are described in WO2014/159595 and WO2011/051351.

### BRIEF DESCRIPTION OF TABLES AND FIGURES

Table 1: Alignment of hNAV1.1 to hNAV1.9, identifying the extracellular loops regions (E1, E2 and E3) for each of the four domains (D1, D2, D3 and D4).

Tables 2 to 6 show various immunisation regimens for producing anti-hNav1.7 antibodies in a mouse system, such as the Kymouse™ system.

Table 7 shows the tabulated data for the 17 antibodies isolated and tested in the DELFIA assay as described in Example 2Aa.

FIG. 1 shows the filter-based time-resolved fluorescence of 17 purified antibodies raised against the KP1.2 peptide of the D2E2 loop of the Nav1.7 channel (SEQ ID No:10). 12 of the 17 samples tested show significant binding to the peptide compared to isotype control (hu IC). Samples were tested at an undiluted concentration.

### SUMMARY OF THE INVENTION

There is a need in the art for using defined E2 loops or peptides to raise antibodies that are able to interfere with the function of VGSCs for therapeutic purposes. Such antibodies would not only provide more specificity than neurotoxin peptides but also confer high accessibility to tissues and stability for clinical use. These defined regions are directly coupled to the function of gating of VGSCs, and thus would greatly help discovery of potent neutralizing antibodies.

These E2 loop contains peptides with 8-22 amino acids. Using peptide immunization, as described previously may not necessarily generate high potent neutralizing antibodies because of inappropriate structure of short peptides and the dynamic structures of these loops in neurons different states of Nav1.7 on neural cells. Such antibodies may not bind to the NAV protein of interest, when in the native transmembrane state. Hence, there is a need in the art for a method of immunization to raise antibodies recognizing the native structure of NAV proteins of interest by binding to these E2 loops and other extracellular loops at the same time to provide sufficient affinity and neutralizing activity, and which are able to provide efficacy by still binding to the native state. Immunizing mice with recombinant isogenic cells (for example, mouse embryonic fibroblasts or human embryonic kidney cells) expressing a NAV protein of interest or DNA encoding NAV protein of interest would permit the identification of antibodies able to recognizing the conformational epitopes, which only present cell surface NAV proteins. However, a combination of such immunization with linear E2 peptide(s)-conjugated carrier would further aid to enrich antibodies binding to E2 loops in their native conformation. Based on the alignment of human VGSCs with NavAb, the inventors have defined the extracellular loops of each of human NAV1.1 to NAV1.9 and mouse NAV1.7.

Antibodies generated using the immunisations as described herein may have a number of benefits over previous antibodies and NAV-targeting drugs. They may be more specific than neurotoxin peptides but also confer high accessibility to tissues and stability in clinical uses. They may be able to bind to one, two or all of NAV1.7, NAV1.8 and NAV1.9, but may not bind to any, or all, of NAV1.1, NAV1.2, NAV1.3, NAV1.4 and NAV1.5. In one embodiment, the antibodies are specific over NAV1.4, NAV1.5 and/or NAV1.6. In another embodiment, the antibodies are specific for one of NAV1.7, NAV1.8 and NAV1.9, over the rest of the NAV proteins.

### DETAILED DESCRIPTION OF THE INVENTION

### Definitions

The term “about” or “approximately” means within 20%, preferably within 10%, and more preferably within 5% (or 1% or less) of a given value or range.

As used herein, “administer” or “administration” refers to the act of injecting or otherwise physically delivering a substance as it exists outside the body (e.g., an anti-NAV protein antibody provided herein) into a patient, by a method or route which results in at least partial delivery of the agent at a desired site, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. Pharmaceutical compositions comprising the compounds disclosed herein can be administered by any appropriate route which results in an effective treatment in the subject.

Multiple compositions can be administered separately or simultaneously. Separate administration refers to the two compositions being administered at different times, e.g. at least 10, 20, 30, or 10-60 minutes apart, or 1, 2, 3, 4, 5, 6, 7, 8, 9, 10, 12 hours apart. One can also administer compositions at 24 hours apart, or even longer apart. Alternatively, two or more compositions can be administered simultaneously, e.g. less than 10 or less than 5 minutes apart. Compositions administered simultaneously can, in some aspects, be administered as a mixture, with or without similar or different time release mechanism for each of the components.

As used herein, an “antagonist” or “inhibitor” of a NAV protein of interest, refers to an antibody or fragment thereof which is capable of inhibiting or otherwise decreasing one or more of the biological activities of the NAV protein of interest, such as in a cell expressing the NAV protein. For example, in certain embodiments, antibodies or fragments thereof are antagonist antibodies that block ion flux in a cell having a cell surface-expressed NAV protein when said antibody is contacted with said cell, resulting in blocking of transmembrane depolarisation. In some embodiments, an antagonist of NAV protein (e.g., an antagonistic anti-NAV antibody as described herein) may, for example, act by inhibiting or otherwise decreasing the activation and/or cell signalling pathways of the cell expressing a NAV protein receptor, thereby inhibiting a NAV protein-mediated biological activity of the cell relative to the NAV protein-mediated biological activity in the absence of antagonist. In certain embodiments, the antibodies provided herein are fully human, antagonistic anti-NAV protein antibodies, preferably fully human, monoclonal, antagonistic anti-NAV protein antibodies.

The term “antibody” and “immunoglobulin” or “Ig” may be used interchangeably herein. For example, an antibody can include a heavy (H) chain variable region (abbreviated herein as V_{H}), and a light (L) chain variable region (abbreviated herein as V_{L}). In another example, an antibody includes two heavy (H) chain variable regions and two light (L) chain variable regions. The antibodies as disclosed herein can be of any type (e.g., IgG, IgE, IgM, IgD and IgA), any class (e.g., IgG1, IgG2, IgG3, IgG4, IgA1 and IgA2, for example human IgG1 or IgG4)), or any subclass (e.g., IgG2a and IgG2b) of immunoglobulin molecule. In preferred embodiments, the NAV protein antibodies are fully human, such as fully human monoclonal anti-NAV protein antibodies. Antibodies can be from any source, including mouse, rabbit, pig, rat, and primate (human and non-human primate) and primatized antibodies. Antibodies also include humanized antibodies, chimeric antibodies, and the like. Antibodies include, but are not limited to, synthetic antibodies, monoclonal antibodies, recombinantly produced antibodies, multispecific antibodies (including bi-specific antibodies), human antibodies, humanized antibodies and chimeric antibodies. Throughout the disclosure, the term “antibody” is intended to also include “antibody fragments”, unless it is clear from the context that such a meaning would be technically meaningless.

An antibody or a fragment thereof that binds to a NAV protein antigen may be cross-reactive with related antigens. Preferably, an antibody or a fragment thereof that binds to a NAV protein antigen does not cross-react with other antigens. An antibody or a fragment thereof that binds to a NAV protein antigen can be identified, for example, by immunoassays, standard Patch Clamp assays (e.g. IonWorks) or other techniques known to those skilled in the art. In one embodiment, the antibodies or fragments as disclosed herein may specifically bind to a NAV protein antigen. An antibody or a fragment thereof binds specifically to a NAV protein antigen when it binds to a NAV protein antigen with higher affinity than to any cross-reactive antigen as determined using experimental techniques, such as radioimmunoassays (RIA), enzyme-linked immunosorbent assays (ELISAs) and standard Patch Clamp assays. Typically, a specific or selective reaction will be at least twice background signal or noise and more typically more than 10 times background. See, e.g., Paul, ed., 1989, Fundamental Immunology Second Edition, Raven Press, New York at pages 332-336 for a discussion regarding antibody specificity.

As used herein, the term “antibody fragment” refers to a polypeptide that includes at least one immunoglobulin variable domain or immunoglobulin variable domain sequence and which binds a given antigen. An antibody fragment can comprise an antibody or a polypeptide comprising an antigen-binding domain of an antibody. In some embodiments, an antibody fragment can comprise a monoclonal antibody or a polypeptide comprising an antigen-binding domain of a monoclonal antibody. The term “antibody fragment” encompasses antigen-binding fragments of antibodies, including single chain antibodies, Fab, Fab′, bispecific Fab and sFab fragments, F(ab′)₂, Fd fragments, Fv fragments, domain antibodies (dAb) fragments (see, e.g. de Wildt et al., Eur 3. Immunol. 1996; 26(3):629-39; which is incorporated by reference herein in its entirety), diabodies, triabodies, midibodies, intrabodies, single-chain Fvs (scFv) (e.g., including monospecific scFvs, bispecific scFv and multispecific scFvs), camelized antibodies e.g. camelised VH, Fab fragments, F(ab′) fragments, disulfide-linked Fvs (dsFv), anti-idiotypic (anti-Id) antibodies, and epitope-binding fragments of any of the above, as well as complete antibodies. In one embodiment, the fragment is selected from a Fab, a Fab′, a F(ab′)2, a bispecific Fab, a dsFv, a camelized VH, a bispecific scFv, a diabody, a triabody and a scFv. In particular, antibody fragments as disclosed herein include immunologically active portions of immunoglobulin molecules, i.e., antigen binding domains or molecules that contain an antigen-binding site that binds to a NAV protein antigen (e.g., one or more complementarity determining regions (CDRs) of an anti-NAV protein antibody).

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. In one embodiment, the amino acid positions assigned to CDRs and FRs may be defined according to Kabat (Sequences of Proteins of Immunological Interest (National Institutes of Health, Bethesda, Md., 1987 and 1991)) or according to IMGT nomenclature.

The term “antigen binding domain,” “antigen binding region,” “antigen binding fragment,” and similar terms, refer to that portion of an antibody which comprises the amino acid residues that interact with an antigen and confer on the binding agent its specificity and affinity for the antigen (e.g., the complementarity determining regions (CDR), such as those defined by Chothia, Kabat or other numbering systems known to those in the art). The antigen binding region can be derived from any animal species, such as rodents (e.g., rabbit, rat or hamster) and humans. In one embodiment, the antigen binding region is of human origin. As used herein, these terms may refer to a polypeptide or domain that comprises one or more CDRs of an antibody which is capable of binding an antigen. For example, the polypeptide comprises a CDR3 (e.g., HCDR3). For example the polypeptide comprises CDRs 1 and 2 (e.g., HCDR1 and 2) or CDRs 1-3 of a variable domain of an antibody (e.g., HCDRs1-3). In an example, the antibody binding site is provided by a single variable domain (e.g., a V_{H} or V_{L} domain). In another example, the binding site comprises a V_{H}/V_{L} pair or two or more of such pairs.

The term “constant region” or “constant domain” refers to a carboxy-terminal portion of the light and heavy chain, which is not directly involved in binding of the antibody to antigen but exhibits various effector functions, such as interaction with the Fc receptor. The terms refer to the portion of an immunoglobulin molecule having a more conserved amino acid sequence relative to the other portion of the immunoglobulin—the variable domain—which contains the antigen binding site. The constant domain contains the CH1, CH2 and CH3 domains of the heavy chain and the CH_{L} domain of the light chain.

As used herein, “corresponding loop” refers to a portion of an isoform of the NAV protein of interest, which, when the sequences are aligned according to the method described in Example 1 below, lies between the same α-domains. A corresponding loop need not be of the same length between isoforms. Table 1 below shows the corresponding loop numbers between isoforms for all of the loops of the human NAV proteins. Alignments may be carried out between the human NAV proteins and NAV proteins of other species to identify the corresponding loop portions.

TABLE 1
definition of corresponding loops between NAV proteins (with reference to SEQ ID NO: 2)

| | | | | |
|---|---|---|---|---|
| NAV protein | E1 | E2 | E3 | Reference sequence |

Domain 1 (D1)

| | | | | |
|---|---|---|---|---|
| 1.1 | 144-151 | 204-211 | 270-397 | SEQ ID No: 56 |
| | (SEQ ID No: 57) | (SEQ ID No: 58) | (SEQ ID No: 59) | |
| 1.2 | 145-152 | 205-212 | 271-399 | SEQ ID No: 74 |
| | (SEQ ID No: 75) | (SEQ ID No: 58) | (SEQ ID No: 77) | |
| 1.3 | 144-151 | 204-211 | 270-398 | SEQ ID No: 92 |
| | (SEQ ID No: 93) | (SEQ ID No: 58) | (SEQ ID No: 95) | |
| 1.4 | 147-154 | 207-214 | 273-421 | SEQ ID No: 110 |
| | (SEQ ID No: 111) | (SEQ ID No: 112) | (SEQ ID No: 113) | |
| 1.5 | 147-154 | 207-214 | 273-387 | SEQ ID No: 128 |
| | (SEQ ID No: 129) | (SEQ ID No: 130) | (SEQ ID No: 131) | |
| 1.6 | 148-155 | 208-215 | 274-385 | SEQ ID No: 146 |
| | (SEQ ID No: 147) | (SEQ ID No: 148) | (SEQ ID No: 149) | |
| 1.7 | 142-149 | 202-209 | 268-376 | SEQ ID No: 2 |
| | (SEQ ID No: 3) | (SEQ ID No: 4) | (SEQ ID No: 5) | |
| 1.8 | 146-153 | 203-210 | 269-371 | SEQ ID No: 20 |
| | (SEQ ID No: 21) | (SEQ ID No: 22) | (SEQ ID No: 23) | |
| 1.9 | 145-152 | 209-217 | 276-374 | SEQ ID No: 38 |
| | (SEQ ID No: 39) | (SEQ ID No: 40) | (SEQ ID No: 41) | |

Domain 2 (D2)

| | | | | |
|---|---|---|---|---|
| 1.1 | 785-797 | 845-854 | 913-966 | SEQ ID No: 56 |
| | (SEQ ID No: 61) | (SEQ ID No: 62) | (SEQ ID No: 63) | |
| 1.2 | 776-788 | 836-845 | 904-957 | SEQ ID No: 74 |
| | (SEQ ID No: 79) | (SEQ ID No: 80) | (SEQ ID No: 81) | |
| 1.3 | 777-789 | 837-846 | 905-958 | SEQ ID No: 92 |
| | (SEQ ID No: 97) | (SEQ ID No: 98) | (SEQ ID No: 99) | |
| 1.4 | 595-607 | 655-664 | 723-776 | SEQ ID No: 110 |
| | (SEQ ID No: 115) | (SEQ ID No: 116) | (SEQ ID No: 117) | |
| 1.5 | 734-746 | 794-803 | 862-913 | SEQ ID No: 128 |
| | (SEQ ID No: 133) | (SEQ ID No: 134) | (SEQ ID No: 135) | |
| 1.6 | 770-782 | 830-839 | 898-951 | SEQ ID No: 146 |
| | (SEQ ID No: 151) | (SEQ ID No: 152) | (SEQ ID No: 153) | |
| 1.7 | 750-762 | 810-819 | 878-931 | SEQ ID No: 2 |
| | (SEQ ID No: 7) | (SEQ ID No: 8) | (SEQ ID No: 9) | |
| 1.8 | 682-694 | 742-751 | 810-864 | SEQ ID No: 20 |
| | (SEQ ID No: 25) | (SEQ ID No: 26) | (SEQ ID No: 27) | |
| 1.9 | 594-606 | 654-665 | 724-785 | SEQ ID No: 38 |
| | (SEQ ID No: 43) | (SEQ ID No: 44) | (SEQ ID No: 45) | |

Domain 3 (D3)

| | | | | |
|---|---|---|---|---|
| 1.1 | 1235-1249 | 1297-1313 | 1370-1457 | SEQ ID No: 56 |
| | (SEQ ID No: 65) | (SEQ ID No: 66) | (SEQ ID No: 67) | |
| 1.2 | 1225-1239 | 1287-1303 | 1360-1447 | SEQ ID No: 74 |
| | (SEQ ID No: 83) | (SEQ ID No: 66) | (SEQ ID No: 85) | |
| 1.3 | 1223-1237 | 1282-1301 | 1358-1442 | SEQ ID No: 92 |
| | (SEQ ID No: 101) | (SEQ ID No: 102) | (SEQ ID No: 103) | |
| 1.4 | 1048-1062 | 1110-1126 | 1183-1269 | SEQ ID No: 110 |
| | (SEQ ID No: 119) | (SEQ ID No: 120) | ( SEQ ID No: 121) | |
| 1.5 | 1222-1236 | 1281-1300 | 1357-1444 | SEQ ID No: 128 |
| | (SEQ ID No: 137) | (SEQ ID No: 138) | (SEQ ID No: 139) | |
| 1.6 | 1215-1229 | 1277-1293 | 1350-1438 | SEQ ID No: 146 |
| | (SEQ ID No: 155) | (SEQ ID No: 156) | (SEQ ID No: 157) | |
| 1.7 | 1198-1212 | 1260-1276 | 1333-1420 | SEQ ID No: 2 |
| | (SEQ ID No: 11) | (SEQ ID No: 12) | (SEQ ID No: 13) | |
| 1.8 | 1169-1183 | 1231-1247 | 1304-1392 | SEQ ID No: 20 |
| | (SEQ ID No: 29) | (SEQ ID No: 30) | (SEQ ID No: 31) | |
| 1.9 | 1073-1087 | 1135-1144 | 1201-1282 | SEQ ID No: 38 |
| | (SEQ ID No: 47) | (SEQ ID No: 48) | (SEQ ID No: 49) | |

Domain 4 (D4)

| | | | | |
|---|---|---|---|---|
| 1.1 | 1558-1571 | 1617-1636 | 1693-1760 | SEQ ID No: 56 |
| | (SEQ ID No: 69) | (SEQ ID No: 70) | (SEQ ID No: 71) | |
| 1.2 | 1548-1561 | 1607-1626 | 1683-1750 | SEQ ID No: 74 |
| | (SEQ ID No: 87) | (SEQ ID No: 70) | (SEQ ID No: 89) | |
| 1.3 | 1543-1556 | 1602-1621 | 1678-1745 | SEQ ID No: 92 |
| | (SEQ ID No: 105) | (SEQ ID No: 106) | (SEQ ID No: 107) | |
| 1.4 | 1370-1380 | 1429-1448 | 1505-1572 | SEQ ID No: 110 |
| | (SEQ ID No: 123) | (SEQ ID No: 124) | (SEQ ID No: 125) | |
| 1.5 | 1545-1558 | 1604-1623 | 1680-1746 | SEQ ID No: 128 |
| | (SEQ ID No: 141) | (SEQ ID No: 142) | (SEQ ID No: 143) | |
| 1.6 | 1539-1552 | 1598-1617 | 1674-1740 | SEQ ID No: 146 |
| | (SEQ ID No: 159) | (SEQ ID No: 160) | (SEQ ID No: 161) | |
| 1.7 | 1521-1534 | 1580-1599 | 1656-1723 | SEQ ID No: 2 |
| | (SEQ ID No: 15) | (SEQ ID No: 16) | (SEQ ID No: 17) | |
| 1.8 | 1493-1506 | 1552-1573 | 1630-1696 | SEQ ID No: 20 |
| | (SEQ ID No: 33) | (SEQ ID No: 34) | (SEQ ID No: 35) | |
| 1.9 | 1383-1396 | 1441-1463 | 1520-1578 | SEQ ID No: 38 |
| | (SEQ ID No: 51) | (SEQ ID No: 52) | (SEQ ID No: 53) | |

The term “epitope” is a region of an antigen (antigenic determinant) which is bound by the antigen binding site of an antibody or fragment thereof (paratope). Different antibodies may bind to different epitopes, and different epitopes may have different biological activities. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, sulfonyl groups, and in certain embodiments may have specific three-dimensional structural characteristics and/or specific charge characteristics. In some embodiments, the epitope is only present or available for binding in the native, cell-surface expressed form of the NAV protein of interest, but is not present or available for binding in the denatured form of the NAV protein of interest. It is thought that some prior art antibodies are able to bind denatured fragments of certain NAV proteins, but are unable to bind to functional NAV proteins in their native state in cell membranes. In some embodiments, the anti-NAV antibodies as described herein bind to functional NAV proteins in their native state in cell membranes. Particular residues comprised within an epitope can be determined through computer modelling programs or via three-dimensional structures obtained through methods known in the art, such as X-ray crystallography. Binding studies to determine the amino acids involved in binding can comprise alanine scanning.

As used herein, the term “in combination” in the context of the administration of other therapies refers to the use of more than one therapy. The use of the term “in combination” does not restrict the order in which therapies are administered to a subject with an infection. A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a NAV-mediated disease. Any additional therapy can be administered in any order with the other additional therapies. In certain embodiments, the anti-NAV antibodies can be administered in combination with one or more therapies (e.g., therapies that are not the antibodies as described herein, and that are currently administered to prevent, treat, manage, and/or ameliorate a NAV protein-mediated disease. Non-limiting examples of therapies that can be administered in combination with an antibody as described herein include analgesic agents, anesthetic agents, antibiotics, or immunomodulatory agents or any other agent listed in the U.S. Pharmacopoeia and/or Physician's Desk Reference. Particular therapies that can be administered in combination with the antibodies described herein include opioid analgesics (e.g. morphine, diamorphine, codeine, dihydrocodeine, fentanyl, oxycodone, buprenorphine, dextropropoxyphene, tramadol, meptazinol, pethidine or pantazocine), paracetamol, non-steroidal anti-inflammatories (e.g. aspirin, ibuprofen, ketoprofen, naproxen, indomethacin, diclofenac, celecoxib, ketorolac, mefenamic acid, meloxicam, piroxicam, nabumetone, parecoxib, sulindac or tenoxicam), local anaesthetics (e.g. bupivacaine, lignocaine), 5HT1 agonists (e.g. sumptriptan or naratriptan), anti-epileptic/antidepressants (e.g. carbamazepine, gabapentin, pregabalin or duloxetine), anxiolytic/muscle relaxants (e.g. diazepam, tizanidine or cyclobenzaprine), ziconitide, botulinum toxin, tetrahydrocannabinol, cannabidiol, capsaicin, anti-NGF drugs, anti-TrkA drugs, anti-CGRP drugs, p75NTR-Fc, TRPV1 antagonists, TRPV3 agonists, voltage-gated sodium channel blockers and FAAH inhibitors.

In the context of a peptide or polypeptide, the term “fragment” as used herein refers to a peptide or polypeptide that comprises less than the full length amino acid sequence. Such a fragment may arise, for example, from a truncation at the amino terminus, a truncation at the carboxy terminus, and/or an internal deletion of one or more residue(s) from the amino acid sequence. Fragments may, for example, result from alternative RNA splicing or from in vivo protease activity. In certain embodiments, fragments of an antibody that binds to a NAV protein of interest include polypeptides comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of an antibody that binds to a NAV protein of interest. In another embodiment, fragments of a NAV protein of interest include polypeptides comprising an amino acid sequence of at least 5 contiguous amino acid residues, at least 6 contiguous amino acid residues, at least 7 contiguous amino acid residues, at least 8 contiguous amino acid residues, at least 9 contiguous amino acid residues, at least 10 contiguous amino acid residues, at least 11 contiguous amino acid residues, at least 12 contiguous amino residues, at least 13 contiguous amino acid residues, at least 14 contiguous amino acid residues, at least 15 contiguous amino acid residues, at least contiguous 16 amino acid residues, at least 17 contiguous amino acid residues, at least 18 contiguous amino acid residues, at least 19 contiguous amino acid residues, at least 20 contiguous amino acid residues, or at least 21 contiguous amino acid residues of the amino acid sequence of a NAV protein of interest. In a specific embodiment, a fragment of a NAV protein of interest or an antibody that binds to a NAV protein of interest retains at least 1, at least 2, or at least 3 functions of the protein or antibody.

The terms “fully human antibody” or “human antibody” are used interchangeably herein, and refer to an antibody that comprises a human variable region and, in one embodiment, a human constant region. In specific embodiments, the terms refer to an antibody that comprises a variable region and constant region of human origin. “Fully human” antibodies include antibodies having the amino acid sequence of a human immunoglobulin and include antibodies isolated from human immunoglobulin libraries or from mice that express antibodies from human genes. “Fully human” anti-NAV protein antibodies, in certain embodiments, can also encompass antibodies which bind NAV proteins which are encoded by nucleic acid sequences which are naturally occurring somatic variants of human germline immunoglobulin nucleic acid sequences. In a specific embodiment, the anti-NAV protein antibodies provided herein are fully human antibodies. The term “fully human antibody” furthermore includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). Exemplary methods of producing fully human antibodies are provided, e.g., in the Examples herein, but any method known in the art may be used.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see e.g., Taylor, L. D. et al., (1992) Nucl. Acids Res. 20:6287-6295) or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “monoclonal antibody” refers to an antibody obtained from a population of homogenous or substantially homogeneous antibodies, and each monoclonal antibody will typically recognize a single epitope on the antigen. In preferred embodiments, a “monoclonal antibody,” as used herein, is an antibody produced by a single hybridoma or other cell, wherein the antibody binds to a NAV protein epitope as determined, e.g., by Patch Clamp assays, ELISA or other antigen-binding or competitive binding assay known in the art or in the Examples provided herein. The term “monoclonal” is not limited to any particular method for making the antibody. For example, monoclonal anti-NAV antibodies as disclosed herein may be made by the hybridoma method as described in Kohler et al.; Nature, 256:495 (1975) or may be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et al., eds., John Wiley and Sons, New York). Other exemplary methods of producing other monoclonal antibodies are provided in the Examples herein.

The term “heavy chain” when used in reference to an antibody refers to five distinct types, called alpha (α), delta (δ), epsilon (ε), gamma (γ) and mu (μ), based on the amino acid sequence of the heavy chain constant domain. These distinct types of heavy chains are well known and give rise to five classes of antibodies, IgA, IgD, IgE, IgG and IgM, respectively, including four subclasses of IgG, namely IgG1, IgG2, IgG3 and IgG4. Preferably the heavy chain is a human heavy chain. In one embodiment, the antibodies as disclosed herein have an isotype selected from IgG, IgE, IgM, IgD, and IgA, such as an isotype selected from IgG1, IgG2, IgG3 and IgG4, for example, the isotype is IgG1 or IgG4, and optionally is IgG2a or IgG2c.

As used herein, “instructions” refers to a display of written, printed or graphic matter on the immediate container of an article, for example the written material displayed on a vial containing a pharmaceutically active agent, or details on the composition and use of a product of interest included in a kit containing a composition of interest. Instructions set forth the method of the treatment as contemplated to be administered or performed.

The term “Kd”, as used herein, is intended to refer to the equilibrium dissociation constant of a particular antibody-antigen interaction. Affinity may be measured, in one embodiment, by Kd.

The term “light chain” when used in reference to an antibody refers to two distinct types, called kappa (κ) of lambda (λ) based on the amino acid sequence of the constant domains. Light chain amino acid sequences are well known in the art. In preferred embodiments, the light chain is a human light chain.

As used herein, “authorization number” or “marketing authorization number” refers to a number issued by a regulatory agency upon that agency determining that a particular medical product and/or composition may be marketed and/or offered for sale in the area under the agency's jurisdiction. As used herein “regulatory agency” refers to one of the agencies responsible for evaluating, e.g, the safety and efficacy of a medical product and/or composition and controlling the sales/marketing of such products and/or compositions in a given area. The Food and Drug Administration (FDA) in the US and the European Medicines Agency (EPA) in Europe are but two examples of such regulatory agencies. Other non-limiting examples can include SDA, MPA, MHPRA, IMA, ANMAT, Hong Kong Department of Health-Drug Office, CDSCO, Medsafe, and KFDA.

A “NAV-mediated disease” and “NAV-mediated condition” are used interchangeably and refer to any disease or condition that is completely or partially caused by or is the result of a NAV protein, e.g. NAV1.1 to NAV1.9, such as NAV1.7, NAV1.8 and/or NAV1.9, and in some embodiments NAV1.7. In certain embodiments, NAV protein is aberrantly (e.g., highly) expressed on the surface of a cell. In some embodiments, NAV protein may be aberrantly upregulated on a particular cell type. In other embodiments, normal, aberrant or excessive ion flux is observed. In certain embodiments, the NAV protein-mediated disease is painful diabetic neuropathy, post-herpetic neuropathy, trigeminal neuralgia, osteoarthritis, chronic back pain, nerve compression pain (e.g. sciatic nerve compression) or cancer pain.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable” as used herein encompasses those substances which are approved by a regulatory agency of the Federal or a state government, or listed in the U.S. Pharmacopeia, European Pharmacopeia or other generally recognized Pharmacopeia for use in animals, and more particularly in humans.

As used herein, the term “polynucleotide,” “nucleotide,” nucleic acid” “nucleic acid molecule” and other similar terms are used interchangeable and include DNA, RNA, mRNA and the like.

The term “therapeutically effective amount” as used herein refers to the amount of a therapy (e.g., an antibody or pharmaceutical composition provided herein) which is sufficient to reduce and/or ameliorate the severity and/or duration of a given disease and/or a symptom related thereto. This term also encompasses an amount necessary for the reduction or amelioration of the advancement or progression of a given disease, reduction or amelioration of the recurrence, development or onset of a given disease, and/or to improve or enhance the prophylactic or therapeutic effect(s) of another therapy (e.g., a therapy other than anti-NAV protein antibody provided herein). In some embodiments, the effective amount of an anti-NAV antibody as disclosed herein is from about 0.1 mg/kg (mg of antibody per kg weight of the subject) to about 100 mg/kg. In certain embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein). In some embodiments, “effective amount” as used herein also refers to the amount of an anti-NAV protein antibody as disclosed herein to achieve a specified result (e.g., inhibition of a NAV protein biological activity of a cell, such as inhibition of ion flux from the cell).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment). For treatment to be effective a complete cure is not contemplated. The method can in certain aspects include cure as well.

The term “variable region” or “variable domain” refers to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in the complimentarily determining regions (CDRs) while the more highly conserved regions in the variable domain are called framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5th ed. (“Kabat et al.”). In preferred embodiments, the variable region is a human variable region.

As used herein the term “comprising” or “comprises” is used in reference to antibodies, fragments, uses, compositions, methods, and respective component(s) thereof, that are essential to the method or composition, yet open to the inclusion of unspecified elements, whether essential or not.

The term “consisting of” refers to antibodies, fragments, uses, compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al, eds.

### Antibodies to NAV1.7

In a first embodiment there is provided an antibody which binds to NAV1.7, particularly to human NAV1.7. Certain anti-NAV1.7 antibodies may bind to conformational epitopes, such as epitopes found on two external loops. Hence, one anti-NAV1.7 antibody according to the invention may be defined according to sentence 1 or sentence 2:

Sentence 1: An antibody or fragment thereof which binds to human NAV1.7 (SEQ ID No:2), and wherein the antibody or fragment binds to:
a. A loop selected from the D1E2 loop (SEQ ID No:4), the D2E2 loop (SEQ ID No:8), the D3E2 loop (SEQ ID No:12) and the D4E2 loop (SEQ ID No:16); and
b. One, two or more (e.g. 3, 4, 5 or 6) of the D1E1 loop (SEQ ID No:3), the D1E3 loop (SEQ ID No:5), the D2E1 loop (SEQ ID No:7), the D2E3 loop (SEQ ID No:9), the D3E1 loop (SEQ ID No:11), the D3E3 loop (SEQ ID No:13), the D4E1 loop (SEQ ID No:15) and the D4E3 loop (SEQ ID No:17).

Sentence 2. An antibody or fragment according to sentence 1, which binds to:
c. the D1E2 loop (SEQ ID No:4); and one or both of the D1E1 loop (SEQ ID No:3) and the D1E3 loop (SEQ ID No:5); or
d. the D2E2 loop (SEQ ID No:8), and one or both of the D2E1 loop (SEQ ID No:7) and the D2E3 loop (SEQ ID No:9); or
e. the D3E2 loop (SEQ ID No:12), and one or both of the D3E1 loop (SEQ ID No:11) and the D3E3 loop (SEQ ID No:13); or
f. the D4E2 loop (SEQ ID No:16), and one or both of the D4E1 loop (SEQ ID No:15) and the D4E3 loop (SEQ ID No:17).

These anti-NAV1.7 antibodies may be cross-reactive with NAV1.8 proteins, e.g. human NAV1.8 proteins. In another embodiment they are cross-reactive with NAV1.9 proteins, e.g. human NAV1.9 proteins. In a further embodiment, they are cross-reactive with both NAV1.8 proteins (e.g. human NAV1.8 protein) and NAV1.9 proteins (e.g. human NAV1.9 protein). In a further embodiment, the antibodies cross react with the corresponding loop sequences in either or both of NAV1.8 and NAV1.9 (see Table 1 hereinabove for SEQ ID NOs for human NAV1.8 and 1.9) in any of the preceding embodiments.

In one embodiment, the anti-NAV1.7 antibodies do not bind (e.g. by SPR) amino acids 202-232 of SEQ ID NO:2 (i.e. do not bind to SEQ ID NO:64). In another embodiment, the anti-NAV1.7 antibodies do not bind amino acids 202-215 of SEQ ID NO:2 (i.e. do not bind to SEQ ID NO:60). In another embodiment, the anti-NAV1.7 antibodies do not bind amino acids 206-211 of SEQ ID NO:2 (i.e. do not bind to SEQ ID NO:68).

Selectivity over other NAV proteins, in particular NAV1.1 to NAV1.6, may provide further benefits in terms of improved treatment of pain diseases or conditions. In another embodiment, selectivity over NAV1.8 and NAV1.9 may be beneficial. Thus, in one embodiment, the anti-NAV1.7 antibodies are selective over any one of NAV1.1, NAV1.2, NAV1.3, NAV1.4, NAV1.5, NAV1.6, NAV1.8 and NAV1.9, e.g. are selective over NAV1.6.

Anti-NAV1.7 antibodies may be selective over NAV1.6. Selectivity may be determined by analysis of certain motifs found with the individual NAV proteins. Methods for determining selectivity is discussed in more detail hereinbelow.

Sentence 90. Thus, in one embodiment, there is provided an anti-NAV1.7 antibody which binds to human NAV1.7 (SEQ ID No:2), and comprises one or both of the features a or b:
a. is selective for a first polypeptide comprising the sequence LTEF (SEQ ID No:169, NAV1.7 motif) over a second polypeptide comprising the sequence ITEF (SEQ ID No:170, NAV1.6 motif); or
b. is selective for a first polypeptide comprising the sequence LTEFVNLGN (SEQ ID No:4, NAV1.7 motif) over a second polypeptide comprising the sequence ITEFVNLGN (SEQ ID No:148, NAV1.6 motif).

Sentence 107. In another embodiment, there is provided an anti-NAV1.7 antibody which binds to human NAV1.7 (SEQ ID No:2), and comprises one or both of the features a or b:
a. is selective for a first polypeptide comprising the sequence VELF (SEQ ID No:176, NAV1.7 motif) over a second polypeptide comprising the sequence MELS (SEQ ID No:177, NAV1.6 motif),
b. is selective for a first polypeptide comprising the sequence VELFLADVEG (SEQ ID No:8, NAV1.7 motif) over a second polypeptide comprising the sequence MELSLADVEG (SEQ ID No:152, NAV1.6 motif);

Sentence 124. In another embodiment, there is provided an anti-NAV1.7 antibody which binds to human NAV1.7 (SEQ ID No:2), and comprises one, two, three, four, five, six, seven or all of the features a. to h.:
a. binds to a first polypeptide comprising the sequence VTLVA (SEQ ID No:193, NAV1.7 motif) over a second polypeptide comprising the sequence VSLIA (SEQ ID No:262, NAV1.6 motif);
b. binds to a first polypeptide comprising the sequence GYSDL (SEQ ID No:198, NAV1.7 motif) over a second polypeptide comprising the sequence GYSEL (SEQ ID No:88, NAV1.6, NAV1.1, NAV1.2, NAV1.3 and NAV1.4 motif);
c. binds to a first polypeptide comprising the sequence TLVANT (SEQ ID No:185, NAV1.7 motif) over a second polypeptide comprising the sequence SLIANA (SEQ ID No:186, NAV1.6 motif);
d. binds to a first polypeptide comprising the sequence TLVAN (SEQ ID No:200, NAV1.7 motif) over a second polypeptide comprising the sequence SLIAN (SEQ ID No:320, NAV1.6 motif);
e. binds to a first polypeptide comprising the sequence SDLGP (SEQ ID No:187, NAV1.7 motif) over a second polypeptide comprising the sequence SELGA (SEQ ID No:188, NAV1.6, NAV1.1, NAV1.2 and NAV1.3 motif);
f. binds to a first polypeptide comprising the sequence TLGYSD (SEQ ID No:191, NAV1.7 motif) over a second polypeptide comprising the sequence ALGYSE (SEQ ID No:192, NAV1.6, NAV1.1, NAV1.2 and NAV1.3 motif);
g. binds to a first polypeptide comprising the sequence TLVANTLGYSDLGP (SEQ ID No:231, NAV1.7 motif) over a second polypeptide comprising the sequence SLIANALGYSELGA (SEQ ID No:327, NAV1.6 motif); and
h. binds to a first polypeptide comprising the sequence VTLVANTLGYSDLGPIK (SEQ ID No:12, NAV1.7 motif) over a second polypeptide comprising the sequence VSLIANALGYSELGAIK (SEQ ID No:156, NAV1.6 motif).

Sentence 141. In another embodiment, there is provided an anti-NAV1.7 antibody which binds to human NAV1.7 (SEQ ID No:2), and comprises one or both of the features a or b:
a. is selective for a first polypeptide comprising the sequence LIET (SEQ ID No:210, NAV1.7 motif) over a second polypeptide comprising the sequence IIEK (SEQ ID No:211, NAV1.6 motif); or
b. is selective for a first polypeptide comprising the sequence VGMFLADLIETYFVSPTLFR (SEQ ID No:16, NAV1.7 motif) over a second polypeptide comprising the sequence VGMFLADIIEKYFVSPTLFR (SEQ ID No:160, NAV1.6 motif).

Anti-NAV1.7 antibodies may be selective over NAV1.1, NAV1.2 and NAV1.3 (due to similarities in the sequences between NAV1.1, NAV1.2 and NAV1.3 in the E2 extracellular loop regions). Selectivity may be determined by analysis of certain motifs found with the individual NAV proteins. Methods for determining selectivity is discussed in more detail hereinbelow.

Sentence 90. Thus, in one embodiment, there is provided an anti-NAV1.7 antibody which binds to human NAV1.7 (SEQ ID No:2), and comprises any one, two, three, four or all of the features c to g:
a. is selective for a first polypeptide comprising the sequence LTEFVN (SEQ ID No:171, NAV1.7 motif) over a second polypeptide comprising the sequence VTEFVD (SEQ ID No:172, NAV1.1, NAV1.2 and NAV1.3 motif);
b. is selective for a first polypeptide comprising the sequence LTEF (SEQ ID No:169, NAV1.7 motif) over a second polypeptide comprising the sequence VTEF (SEQ ID No:240, NAV1.1, NAV1.2 and NAV1.3 motif);
c. is selective for a first polypeptide comprising the sequence LTEFV (SEQ ID No:305, NAV1.7 motif) over a second polypeptide comprising the sequence VTEFV (SEQ ID No:303, NAV1.1, NAV1.2 and NAV1.3 motif);
d. is selective for a first polypeptide comprising the sequence LTEFVNLGN (SEQ ID No:4, NAV1.7 motif) over a second polypeptide comprising the sequence VTEFVDLGN (SEQ ID No:58, NAV1.1, NAV1.2 and NAV1.3 motif) and
e. is selective for a first polypeptide comprising the sequence FVNLG (SEQ ID No:173, NAV1.7 motif) over a second polypeptide comprising the sequence FVDLG (SEQ ID No:174, NAV1.1, NAV1.2, NAV1.3, NAV1.4 and NAV1.5 motif).

Anti-NAV1.7 antibodies may be selective over NAV1.4. Selectivity may be determined by analysis of certain motifs found with the individual NAV proteins. Methods for determining selectivity is discussed in more detail hereinbelow.

Sentence 90. Thus, in one embodiment, there is provided an anti-NAV1.7 antibody which binds to human NAV1.7 (SEQ ID No:2), and comprises one, two or all of the features g to i:
g. is selective for a first polypeptide comprising the sequence FVNLG (SEQ ID No:173, NAV1.7 motif) over a second polypeptide comprising the sequence FVDLG (SEQ ID No:174, NAV1.1, NAV1.2, NAV1.3, NAV1.4 and NAV1.5 motif);
h. is selective for a first polypeptide comprising the sequence LTEFVN (SEQ ID No:171, NAV1.7 motif) over a second polypeptide comprising the sequence LTEFVD (SEQ ID No:239, NAV1.4 motif); and
i. is selective for a first polypeptide comprising the sequence LTEFVNLGN (SEQ ID No:4, NAV1.7 motif) over a second polypeptide comprising the sequence LTEFVDLGN (SEQ ID No:112, NAV1.4 motif).

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.7 antibodies are selective over NAV1.4 and NAV1.5. In a further embodiment, the anti-NAV1.7 antibodies are selective over NAV1.4 and NAV1.6. In a further embodiment, the anti-NAV1.7 antibodies are selective over NAV1.5 and NAV1.6. In a further embodiment, the anti-NAV1.7 antibodies are selective over NAV1.4, NAV1.5 and NAV1.6.

In one aspect, the anti-NAV1.7 antibodies are selective over at least two other NAV proteins. Thus, in a further embodiment, the anti-NAV1.7 antibodies are selective over NAV1.1 and NAV1.2. In a further embodiment, the anti-NAV1.7 antibodies are selective over NAV1.1 and NAV1.3. In a further embodiment, the anti-NAV1.7 antibodies are selective over NAV1.1 and NAV1.4. In a further embodiment, the anti-NAV1.7 antibodies are selective over NAV1.1 and NAV1.5. In a further embodiment, the anti-NAV1.7 antibodies are selective over NAV1.1 and NAV1.6. In a further embodiment, the anti-NAV1.7 antibodies are selective over NAV1.2 and NAV1.3. In a further embodiment, the anti-NAV1.7 antibodies are selective over NAV1.2 and NAV1.4. In a further embodiment, the anti-NAV1.7 antibodies are selective over NAV1.2 and NAV1.5. In a further embodiment, the anti-NAV1.7 antibodies are selective over NAV1.2 and NAV1.6. In a further embodiment, the anti-NAV1.7 antibodies are selective over NAV1.3 and NAV1.4. In a further embodiment, the anti-NAV1.7 antibodies are selective over NAV1.3 and NAV1.5. In a further embodiment, the anti-NAV1.7 antibodies are selective over NAV1.3 and NAV1.6.

In one aspect, the anti-NAV1.7 antibodies are selective over at least three other NAV proteins. Thus, in a further embodiment, the anti-NAV1.7 antibodies are selective over NAV1.1, NAV1.2 and NAV1.3. In a further embodiment, the anti-NAV1.7 antibodies are selective over NAV1.1, NAV1.2 and NAV1.4. In a further embodiment, the anti-NAV1.7 antibodies are selective over NAV1.1, NAV1.2 and NAV1.5. In a further embodiment, the anti-NAV1.7 antibodies are selective over NAV1.1, NAV1.2 and NAV1.6. In a further embodiment, the anti-NAV1.7 antibodies are selective over NAV1.1, NAV1.3 and NAV1.4. In a further embodiment, the anti-NAV1.7 antibodies are selective over NAV1.1, NAV1.3 and NAV1.5. In a further embodiment, the anti-NAV1.7 antibodies are selective over NAV1.1, NAV1.3 and NAV1.6. In a further embodiment, the anti-NAV1.7 antibodies are selective over NAV1.1, NAV1.4 and NAV1.5. In a further embodiment, the anti-NAV1.7 antibodies are selective over NAV1.1, NAV1.4 and NAV1.6. In a further embodiment, the anti-NAV1.7 antibodies are selective over NAV1.2, NAV1.3 and NAV1.4. In a further embodiment, the anti-NAV1.7 antibodies are selective over NAV1.2, NAV1.3 and NAV1.5. In a further embodiment, the anti-NAV1.7 antibodies are selective over NAV1.2, NAV1.3 and NAV1.6. In a further embodiment, the anti-NAV1.7 antibodies are selective over NAV1.3, NAV1.4 and NAV1.5. In a further embodiment, the anti-NAV1.7 antibodies are selective over NAV1.3, NAV1.4 and NAV1.6.

In a further embodiment, the anti-NAV1.7 antibodies are selective over all of NAV1.1, NAV1.2, NAV1.3, NAV1.4, NAV1.5 and NAV1.6.

In order to be useful in in vivo models of pain, it is advantageous for the anti-NAV1.7 antibodies as disclosed herein to be cross-reactive with NAV1.7 proteins from other species, in particular rat, mouse and monkey (e.g. cynomolgus). Thus, in one embodiment, the anti-NAV1.7 antibodies are cross-reactive with rat NAV1.7 protein (SEQ ID NO: 163). In another embodiment, the anti-NAV1.7 antibodies are cross-reactive mouse NAV1.7 protein (SEQ ID NO:72). In another embodiment, the anti-NAV1.7 antibodies are cross-reactive cyno NAV1.7 protein (SEQ ID NO:164). In a further embodiment, the anti-NAV1.7 antibodies are cross-reactive with both mouse NAV1.7 and rat NAV1.7. In a further embodiment, the anti-NAV1.7 antibodies are cross-reactive with both mouse NAV1.7 and cyno NAV1.7. In a further embodiment, the anti-NAV1.7 antibodies are cross-reactive with both cyno NAV1.7 and rat NAV1.7. In a further embodiment, the anti-NAV1.7 antibodies are cross-reactive with each of cyno NAV1.7, rat NAV1.7 and mouse NAV1.7. In a further embodiment, the antibodies are cross-reactive with the corresponding loop sequences in rat NAV1.7, mouse NAV1.7 and/or cyno NAV1.7 in any of the preceding embodiments.

### Antibodies to NAV1.8

In a second embodiment there is provided an antibody which binds to NAV1.8, particularly to human NAV1.8. Certain anti-NAV1.8 antibodies may bind to conformational epitopes, such as epitopes found on two external loops. Hence, one anti-NAV1.8 antibody according to the invention may be defined according to sentence 14 or sentence 15:

Sentence 14. An antibody or fragment thereof which binds to human NAV1.8 (SEQ ID No:20), and wherein the antibody or fragment binds to:
a. A loop selected from the D1E2 loop (SEQ ID No:22), the D2E2 loop (SEQ ID No:26), the D3E2 loop (SEQ ID No:30) and the D4E2 loop (SEQ ID No:34); and
b. One, two or more (e.g. 3, 4, 5 or 6) of the D1E1 loop (SEQ ID No:21), the D1E3 loop (SEQ ID No:23), the D2E1 loop (SEQ ID No:25), the D2E3 loop (SEQ ID No:27), the D3E1 loop (SEQ ID No:29), the D3E3 loop (SEQ ID No:31), the D4E1 loop (SEQ ID No:33) and the D4E3 loop (SEQ ID No:35).

Sentence 15. An antibody or fragment according to sentence 14, which binds to:
a. the D1E2 loop (SEQ ID No:22); and one or both of the D1E1 loop (SEQ ID No:21) and the D1E3 loop (SEQ ID No:23); or the D2E2 loop (SEQ ID No:26), and one or both of the D2E1 loop (SEQ ID No:25) and the D2E3 loop (SEQ ID No:27); or
b. the D3E2 loop (SEQ ID No:30), and one or both of the D3E1 loop (SEQ ID No:29) and the D3E3 loop (SEQ ID No:31); or
c. the D4E2 loop (SEQ ID No:34), and one or both of the D4E1 loop (SEQ ID No:33) and the D4E3 loop (SEQ ID No:35).

These anti-NAV1.8 antibodies may be cross-reactive with NAV1.7 proteins, e.g. human NAV1.7 proteins. In another embodiment they are cross-reactive with NAV1.9 proteins, e.g. human NAV1.9 proteins. In a further embodiment, they are cross-reactive with both NAV1.7 proteins (e.g. human NAV1.7 protein) and NAV1.9 proteins (e.g. human NAV1.9 protein). In a further embodiment, the antibodies cross react with the corresponding loop sequences in either or both of NAV1.7 and NAV1.9 (see Table 1 hereinabove for SEQ ID NOs for human NAV1.7 and 1.9) in any of the preceding embodiments.

Selectivity over other NAV proteins, in particular NAV1.1 to NAV1.6, may provide further benefits in terms of improved treatment of pain diseases or conditions. In another embodiment, selectivity over NAV1.7 and NAV1.9 may be beneficial. Thus, in one embodiment, the anti-NAV1.8 antibodies are selective over any one of NAV1.1, NAV1.2, NAV1.3, NAV1.4, NAV1.5, NAV1.6, NAV1.7 and NAV1.9, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.8 antibodies are selective over NAV1.4 and NAV1.5. In a further embodiment, the anti-NAV1.8 antibodies are selective over NAV1.4 and NAV1.6. In a further embodiment, the anti-NAV1.8 antibodies are selective over NAV1.5 and NAV1.6. In a further embodiment, the anti-NAV1.8 antibodies are selective over NAV1.4, NAV1.5 and NAV1.6.

In one aspect, the anti-NAV1.8 antibodies are selective over at least two other NAV proteins. Thus, in a further embodiment, the anti-NAV1.8 antibodies are selective over NAV1.1 and NAV1.2. In a further embodiment, the anti-NAV1.8 antibodies are selective over NAV1.1 and NAV1.3. In a further embodiment, the anti-NAV1.8 antibodies are selective over NAV1.1 and NAV1.4. In a further embodiment, the anti-NAV1.8 antibodies are selective over NAV1.1 and NAV1.5. In a further embodiment, the anti-NAV1.8 antibodies are selective over NAV1.1 and NAV1.6. In a further embodiment, the anti-NAV1.8 antibodies are selective over NAV1.2 and NAV1.3. In a further embodiment, the anti-NAV1.8 antibodies are selective over NAV1.2 and NAV1.4. In a further embodiment, the anti-NAV1.8 antibodies are selective over NAV1.2 and NAV1.5. In a further embodiment, the anti-NAV1.8 antibodies are selective over NAV1.2 and NAV1.6. In a further embodiment, the anti-NAV1.8 antibodies are selective over NAV1.3 and NAV1.4. In a further embodiment, the anti-NAV1.8 antibodies are selective over NAV1.3 and NAV1.5. In a further embodiment, the anti-NAV1.8 antibodies are selective over NAV1.3 and NAV1.6.

In one aspect, the anti-NAV1.8 antibodies are selective over at least three other NAV proteins. Thus, in a further embodiment, the anti-NAV1.8 antibodies are selective over NAV1.1, NAV1.2 and NAV1.3. In a further embodiment, the anti-NAV1.8 antibodies are selective over NAV1.1, NAV1.2 and NAV1.4. In a further embodiment, the anti-NAV1.8 antibodies are selective over NAV1.1, NAV1.2 and NAV1.5. In a further embodiment, the anti-NAV1.8 antibodies are selective over NAV1.1, NAV1.2 and NAV1.6. In a further embodiment, the anti-NAV1.8 antibodies are selective over NAV1.1, NAV1.3 and NAV1.4. In a further embodiment, the anti-NAV1.8 antibodies are selective over NAV1.1, NAV1.3 and NAV1.5. In a further embodiment, the anti-NAV1.8 antibodies are selective over NAV1.1, NAV1.3 and NAV1.6. In a further embodiment, the anti-NAV1.8 antibodies are selective over NAV1.1, NAV1.4 and NAV1.5. In a further embodiment, the anti-NAV1.8 antibodies are selective over NAV1.1, NAV1.4 and NAV1.6. In a further embodiment, the anti-NAV1.8 antibodies are selective over NAV1.2, NAV1.3 and NAV1.4. In a further embodiment, the anti-NAV1.8 antibodies are selective over NAV1.2, NAV1.3 and NAV1.5. In a further embodiment, the anti-NAV1.8 antibodies are selective over NAV1.2, NAV1.3 and NAV1.6. In a further embodiment, the anti-NAV1.8 antibodies are selective over NAV1.3, NAV1.4 and NAV1.5. In a further embodiment, the anti-NAV1.8 antibodies are selective over NAV1.3, NAV1.4 and NAV1.6.

In a further embodiment, the anti-NAV1.8 antibodies are selective over all of NAV1.1, NAV1.2, NAV1.3, NAV1.4, NAV1.5 and NAV1.6.

In order to be useful in in vivo models of pain, it is advantageous for the anti-NAV1.8 antibodies as disclosed herein to be cross-reactive with NAV1.8 proteins from other species, in particular rat, mouse and monkey (e.g. cynomolgus). Thus, in one embodiment, the anti-NAV1.8 antibodies are cross-reactive with rat NAV1.8 protein (SEQ ID NO: 165). In another embodiment, the anti-NAV1.8 antibodies are cross-reactive mouse NAV1.8 protein (SEQ ID NO:78). In another embodiment, the anti-NAV1.8 antibodies are cross-reactive cyno NAV1.8 protein (SEQ ID NO:166). In a further embodiment, the anti-NAV1.8 antibodies are cross-reactive with both mouse NAV1.8 and rat NAV1.8. In a further embodiment, the anti-NAV1.8 antibodies are cross-reactive with both mouse NAV1.8 and cyno NAV1.8. In a further embodiment, the anti-NAV1.8 antibodies are cross-reactive with both cyno NAV1.8 and rat NAV1.8. In a further embodiment, the anti-NAV1.8 antibodies are cross-reactive with each of cyno NAV1.8, rat NAV1.8 and mouse NAV1.8. In a further embodiment, the antibodies are cross-reactive with the corresponding loop sequences in rat NAV1.8, mouse NAV1.8 and/or cyno NAV1.8 in any of the preceding embodiments.

### Antibodies to NAV1.9

In a third embodiment there is provided an antibody which binds to NAV1.9, particularly to human NAV1.9. Certain anti-NAV1.9 antibodies may bind to conformational epitopes, such as epitopes found on two external loops. Hence, one anti-NAV1.9 antibody according to the invention may be defined according to sentence 27 or sentence 28:

Sentence 27. An antibody or fragment thereof which binds to human NAV1.9 (SEQ ID No:38), and wherein the antibody or fragment binds to:
a. A loop selected from the D1E2 loop (SEQ ID No:40), the D2E2 loop (SEQ ID No:44), the D3E2 loop (SEQ ID No:48) and the D4E2 loop (SEQ ID No:52); and
b. One, two or more (e.g. 3, 4, 5 or 6) of the D1E1 loop (SEQ ID No:39), the D1E3 loop (SEQ ID No:41), the D2E1 loop (SEQ ID No:43), the D2E3 loop (SEQ ID No:45), the D3E1 loop (SEQ ID No:47), the D3E3 loop (SEQ ID No:49), the D4E1 loop (SEQ ID No:51) and the D4E3 loop (SEQ ID No:53).

Sentence 28. An antibody or fragment according to sentence 27, which binds to:
a. the D1E2 loop (SEQ ID No:40); and one or both of the D1E1 loop (SEQ ID No:39) and the D1E3 loop (SEQ ID No:41); or
b. the D2E2 loop (SEQ ID No:44), and one or both of the D2E1 loop (SEQ ID No:43) and the D2E3 loop (SEQ ID No:45); or
c. the D3E2 loop (SEQ ID No:48), and one or both of the D3E1 loop (SEQ ID No:47) and the D3E3 loop (SEQ ID No:49); or
d. the D4E2 loop (SEQ ID No:52), and one or both of the D4E1 loop (SEQ ID No:51) and the D4E3 loop (SEQ ID No:53).

These anti-NAV1.9 antibodies may be cross-reactive with NAV1.7 proteins, e.g. human NAV1.7 proteins. In another embodiment they are cross-reactive with NAV1.8 proteins, e.g. human NAV1.8 proteins. In a further embodiment, they are cross-reactive with both NAV1.7 proteins (e.g. human NAV1.7 protein) and NAV1.8 proteins (e.g. human NAV1.8 protein). In a further embodiment, the antibodies cross react with the corresponding loop sequences in either or both of NAV1.7 and NAV1.8 (see Table 1 hereinabove for SEQ ID NOs for human NAV1.7 and 1.8) in any of the preceding embodiments.

Selectivity over other NAV proteins, in particular NAV1.1 to NAV1.6, may provide further benefits in terms of improved treatment of pain diseases or conditions. In another embodiment, selectivity over NAV1.7 and NAV1.8 may be beneficial. Thus, in one embodiment, the anti-NAV1.9 antibodies are selective over any one of NAV1.1, NAV1.2, NAV1.3, NAV1.4, NAV1.5, NAV1.6, NAV1.7 and NAV1.8, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.9 antibodies are selective over NAV1.4 and NAV1.5. In a further embodiment, the anti-NAV1.9 antibodies are selective over NAV1.4 and NAV1.6. In a further embodiment, the anti-NAV1.9 antibodies are selective over NAV1.5 and NAV1.6. In a further embodiment, the anti-NAV1.9 antibodies are selective over NAV1.4, NAV1.5 and NAV1.6.

In one aspect, the anti-NAV1.9 antibodies are selective over at least two other NAV proteins. Thus, in a further embodiment, the anti-NAV1.9 antibodies are selective over NAV1.1 and NAV1.2. In a further embodiment, the anti-NAV1.9 antibodies are selective over NAV1.1 and NAV1.3. In a further embodiment, the anti-NAV1.9 antibodies are selective over NAV1.1 and NAV1.4. In a further embodiment, the anti-NAV1.9 antibodies are selective over NAV1.1 and NAV1.5. In a further embodiment, the anti-NAV1.9 antibodies are selective over NAV1.1 and NAV1.6. In a further embodiment, the anti-NAV1.9 antibodies are selective over NAV1.2 and NAV1.3. In a further embodiment, the anti-NAV1.9 antibodies are selective over NAV1.2 and NAV1.4. In a further embodiment, the anti-NAV1.9 antibodies are selective over NAV1.2 and NAV1.5. In a further embodiment, the anti-NAV1.9 antibodies are selective over NAV1.2 and NAV1.6. In a further embodiment, the anti-NAV1.9 antibodies are selective over NAV1.3 and NAV1.4. In a further embodiment, the anti-NAV1.9 antibodies are selective over NAV1.3 and NAV1.5. In a further embodiment, the anti-NAV1.9 antibodies are selective over NAV1.3 and NAV1.6.

In one aspect, the anti-NAV1.9 antibodies are selective over at least three other NAV proteins. Thus, in a further embodiment, the anti-NAV1.9 antibodies are selective over NAV1.1, NAV1.2 and NAV1.3. In a further embodiment, the anti-NAV1.9 antibodies are selective over NAV1.1, NAV1.2 and NAV1.4. In a further embodiment, the anti-NAV1.9 antibodies are selective over NAV1.1, NAV1.2 and NAV1.5. In a further embodiment, the anti-NAV1.9 antibodies are selective over NAV1.1, NAV1.2 and NAV1.6. In a further embodiment, the anti-NAV1.9 antibodies are selective over NAV1.1, NAV1.3 and NAV1.4. In a further embodiment, the anti-NAV1.9 antibodies are selective over NAV1.1, NAV1.3 and NAV1.5. In a further embodiment, the anti-NAV1.9 antibodies are selective over NAV1.1, NAV1.3 and NAV1.6. In a further embodiment, the anti-NAV1.9 antibodies are selective over NAV1.1, NAV1.4 and NAV1.5. In a further embodiment, the anti-NAV1.9 antibodies are selective over NAV1.1, NAV1.4 and NAV1.6. In a further embodiment, the anti-NAV1.9 antibodies are selective over NAV1.2, NAV1.3 and NAV1.4. In a further embodiment, the anti-NAV1.9 antibodies are selective over NAV1.2, NAV1.3 and NAV1.5. In a further embodiment, the anti-NAV1.9 antibodies are selective over NAV1.2, NAV1.3 and NAV1.6. In a further embodiment, the anti-NAV1.9 antibodies are selective over NAV1.3, NAV1.4 and NAV1.5. In a further embodiment, the anti-NAV1.9 antibodies are selective over NAV1.3, NAV1.4 and NAV1.6.

In a further embodiment, the anti-NAV1.9 antibodies are selective over all of NAV1.1, NAV1.2, NAV1.3, NAV1.4, NAV1.5 and NAV1.6.

In order to be useful in in vivo models of pain, it is advantageous for the anti-NAV1.9 antibodies as disclosed herein to be cross-reactive with NAV1.9 proteins from other species, in particular rat, mouse and monkey (e.g. cynomolgus). Thus, in one embodiment, the anti-NAV1.9 antibodies are cross-reactive with rat NAV1.9 protein (SEQ ID NO: 167). In another embodiment, the anti-NAV1.9 antibodies are cross-reactive mouse NAV1.9 protein (SEQ ID NO:82). In another embodiment, the anti-NAV1.9 antibodies are cross-reactive cyno NAV1.9 protein (SEQ ID NO:168). In a further embodiment, the anti-NAV1.9 antibodies are cross-reactive with both mouse NAV1.9 and rat NAV1.9. In a further embodiment, the anti-NAV1.9 antibodies are cross-reactive with both mouse NAV1.9 and cyno NAV1.9. In a further embodiment, the anti-NAV1.9 antibodies are cross-reactive with both cyno NAV1.9 and rat NAV1.9. In a further embodiment, the anti-NAV1.9 antibodies are cross-reactive with each of cyno NAV1.9, rat NAV1.9 and mouse NAV1.9. In a further embodiment, the antibodies are cross-reactive with the corresponding loop sequences in rat NAV1.9, mouse NAV1.9 and/or cyno NAV1.9 in any of the preceding embodiments.

### Antibodies to NAV1.7 and NAV1.8

In a fourth embodiment there is provided an antibody which binds to NAV1.7 and NAV1.8, particularly to human NAV1.7 and human NAV1.8. Certain anti-NAV1.7 and NAV1.8 cross-reactive antibodies may bind to conformational epitopes, such as epitopes found on two external loops. Hence, one anti-NAV1.7 and NAV1.8 cross-reactive antibody according to the invention may be defined according to sentence 40 or sentence 41:

Sentence 40. An antibody or fragment thereof which binds to human NAV1.7 (SEQ ID No:2), and wherein the antibody or fragment binds to:
a. A loop selected from the D1E2 loop (SEQ ID No:4), the D2E2 loop (SEQ ID No:8), the D3E2 loop (SEQ ID No:12) and the D4E2 loop (SEQ ID No:16); and
b. One, two or more (e.g. 3, 4, 5 or 6) of the D1E1 loop (SEQ ID No:3), the D1E3 loop (SEQ ID No:5), the D2E1 loop (SEQ ID No:7), the D2E3 loop (SEQ ID No:9), the D3E1 loop (SEQ ID No:11), the D3E3 loop (SEQ ID No:13), the D4E1 loop (SEQ ID No:15) and the D4E3 loop (SEQ ID No:17); and
   which binds to human NAV1.8 (SEQ ID No:20), and wherein the antibody or fragment binds to:
c. A loop selected from the D1E2 loop (SEQ ID No:22), the D2E2 loop (SEQ ID No:26), the D3E2 loop (SEQ ID No:30) and the D4E2 loop (SEQ ID No:34); and
d. One two or more (e.g. 3, 4, 5 or 6) of the D1E1 loop (SEQ ID No:21), the D1E3 loop (SEQ ID No:23), the D2E1 loop (SEQ ID No:25), the D2E3 loop (SEQ ID No:27), the D3E1 loop (SEQ ID No:29), the D3E3 loop (SEQ ID No:31), the D4E1 loop (SEQ ID No:33) and the D4E3 loop (SEQ ID No:35).

Sentence 41. An antibody or fragment according to sentence 40, which binds to human NAV1.7 (SEQ ID No:2), and wherein the antibody or fragment binds to:
a. The D1E2 loop (SEQ ID No:4); and one or both of the D1E1 loop (SEQ ID No:3) and the D1E3 loop (SEQ ID No:5); or
b. the D2E2 loop (SEQ ID No:8), and one or both of the D2E1 loop (SEQ ID No:7) and the D2E3 loop (SEQ ID No:9); or
c. the D3E2 loop (SEQ ID No:12), and one or both of the D3E1 loop (SEQ ID No:11) and the D3E3 loop (SEQ ID No:13); or
d. the D4E2 loop (SEQ ID No:16), and one or both of the D4E1 loop (SEQ ID No:15) and the D4E3 loop (SEQ ID No:17); and
   which binds to human NAV1.8 (SEQ ID No:20), and wherein the antibody or fragment binds to:
e. the D1E2 loop (SEQ ID No:22); and one or both of the D1E1 loop (SEQ ID No:21) and the D1E3 loop (SEQ ID No:23); or
f. the D2E2 loop (SEQ ID No:26), and one or both of the D2E1 loop (SEQ ID No:25) and the D2E3 loop (SEQ ID No:27); or
g. the D3E2 loop (SEQ ID No:30), and one or both of the D3E1 loop (SEQ ID No:29) and the D3E3 loop (SEQ ID No:31); or
h. the D4E2 loop (SEQ ID No:34), and one or both of the D4E1 loop (SEQ ID No:33) and the D4E3 loop (SEQ ID No:35).

These anti-NAV1.7 and NAV1.8 cross-reactive antibodies may be cross-reactive with NAV1.9 proteins, e.g. human NAV1.9 proteins. In a further embodiment, the antibodies cross react with the corresponding loop sequences in NAV1.9 (see Table 1 hereinabove for SEQ ID NOs for human NAV1.9) in any of the preceding embodiments.

Selectivity over other NAV proteins, in particular NAV1.1 to NAV1.6, may provide further benefits in terms of improved treatment of pain diseases or conditions. In another embodiment, selectivity over NAV1.9 may be beneficial. Thus, in one embodiment, the anti-NAV1.7 and NAV1.8 cross-reactive antibodies are selective over any one of NAV1.1, NAV1.2, NAV1.3, NAV1.4, NAV1.5, NAV1.6 and NAV1.9, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.7 and NAV1.8 cross-reactive antibodies are selective over NAV1.4 and NAV1.5. In a further embodiment, the anti-NAV1.7 and NAV1.8 cross-reactive antibodies are selective over NAV1.4 and NAV1.6. In a further embodiment, the anti-NAV1.7 and NAV1.8 cross-reactive antibodies are selective over NAV1.5 and NAV1.6. In a further embodiment, the anti-NAV1.7 and NAV1.8 cross-reactive antibodies are selective over NAV1.4, NAV1.5 and NAV1.6.

In one aspect, the anti-NAV1.7 and NAV1.8 cross-reactive antibodies are selective over at least two other NAV proteins. Thus, in a further embodiment, the anti-NAV1.7 and NAV1.8 cross-reactive antibodies are selective over NAV1.1 and NAV1.2. In a further embodiment, the anti-NAV1.7 and NAV1.8 antibodies are selective over NAV1.1 and NAV1.3. In a further embodiment, the anti-NAV1.7 and NAV1.8 cross-reactive antibodies are selective over NAV1.1 and NAV1.4. In a further embodiment, the anti-NAV1.7 and NAV1.8 cross-reactive antibodies are selective over NAV1.1 and NAV1.5. In a further embodiment, the anti-NAV1.7 and NAV1.8 cross-reactive antibodies are selective over NAV1.1 and NAV1.6. In a further embodiment, the anti-NAV1.7 and NAV1.8 cross-reactive antibodies are selective over NAV1.2 and NAV1.3. In a further embodiment, the anti-NAV1.7 and NAV1.8 cross-reactive antibodies are selective over NAV1.2 and NAV1.4. In a further embodiment, the anti-NAV1.7 and NAV1.8 cross-reactive antibodies are selective over NAV1.2 and NAV1.5. In a further embodiment, the anti-NAV1.7 and NAV1.8 cross-reactive antibodies are selective over NAV1.2 and NAV1.6. In a further embodiment, the anti-NAV1.7 and NAV1.8 cross-reactive antibodies are selective over NAV1.3 and NAV1.4. In a further embodiment, the anti-NAV1.7 and NAV1.8 cross-reactive antibodies are selective over NAV1.3 and NAV1.5. In a further embodiment, the anti-NAV1.7 and NAV1.8 cross-reactive antibodies are selective over NAV1.3 and NAV1.6.

In one aspect, the anti-NAV1.7 and NAV1.8 cross-reactive antibodies are selective over at least three other NAV proteins. Thus, in a further embodiment, the anti-NAV1.7 and NAV1.8 cross-reactive antibodies are selective over NAV1.1, NAV1.2 and NAV1.3. In a further embodiment, the anti-NAV1.7 and NAV1.8 cross-reactive antibodies are selective over NAV1.1, NAV1.2 and NAV1.4. In a further embodiment, the anti-NAV1.7 and NAV1.8 cross-reactive antibodies are selective over NAV1.1, NAV1.2 and NAV1.5. In a further embodiment, the anti-NAV1.7 and NAV1.8 cross-reactive antibodies are selective over NAV1.1, NAV1.2 and NAV1.6. In a further embodiment, the anti-NAV1.7 and NAV1.8 cross-reactive antibodies are selective over NAV1.1, NAV1.3 and NAV1.4. In a further embodiment, the anti-NAV1.7 and NAV1.8 cross-reactive antibodies are selective over NAV1.1, NAV1.3 and NAV1.5. In a further embodiment, the anti-NAV1.7 and NAV1.8 cross-reactive antibodies are selective over NAV1.1, NAV1.3 and NAV1.6. In a further embodiment, the anti-NAV1.7 and NAV1.8 cross-reactive antibodies are selective over NAV1.1, NAV1.4 and NAV1.5. In a further embodiment, the anti-NAV1.7 and NAV1.8 cross-reactive antibodies are selective over NAV1.1, NAV1.4 and NAV1.6. In a further embodiment, the anti-NAV1.7 and NAV1.8 cross-reactive antibodies are selective over NAV1.2, NAV1.3 and NAV1.4. In a further embodiment, the anti-NAV1.7 and NAV1.8 cross-reactive antibodies are selective over NAV1.2, NAV1.3 and NAV1.5. In a further embodiment, the anti-NAV1.7 and NAV1.8 cross-reactive antibodies are selective over NAV1.2, NAV1.3 and NAV1.6. In a further embodiment, the anti-NAV1.7 and NAV1.8 cross-reactive antibodies are selective over NAV1.3, NAV1.4 and NAV1.5. In a further embodiment, the anti-NAV1.7 and NAV1.8 cross-reactive antibodies are selective over NAV1.3, NAV1.4 and NAV1.6.

In a further embodiment, the anti-NAV1.7 and NAV1.8 cross-reactive antibodies are selective over all of NAV1.1, NAV1.2, NAV1.3, NAV1.4, NAV1.5 and NAV1.6.

In order to be useful in in vivo models of pain, it is advantageous for the anti-NAV1.7 and NAV1.8 cross-reactive antibodies as disclosed herein to be additionally cross-reactive with NAV1.7 and NAV1.8 proteins from other species, in particular rat, mouse and monkey (e.g. cynomolgus). Thus, in one embodiment, the anti-NAV1.7 and NAV1.8 cross-reactive antibodies are also cross-reactive with rat NAV1.7 (SEQ ID No:163). In another embodiment, the anti-NAV1.7 and NAV1.8 cross-reactive antibodies are also cross-reactive mouse NAV1.7 (SEQ ID No:72). In another embodiment, the anti-NAV1.7 and NAV1.8 cross-reactive antibodies are also cross-reactive with cyno NAV1.7 (SEQ ID No:164). In another embodiment, the anti-NAV1.7 and NAV1.8 cross-reactive antibodies are also cross-reactive with rat NAV1.8 (SEQ ID No:165). In another embodiment, the anti-NAV1.7 and NAV1.8 cross-reactive antibodies are also cross-reactive with cyno NAV1.8 (SEQ ID No:166). In another embodiment, the anti-NAV1.7 and NAV1.8 cross-reactive antibodies are also cross-reactive with mouse NAV1.8 (SEQ ID No:78). In a further embodiment, the anti-NAV1.7 and NAV1.8 cross-reactive antibodies are also cross-reactive with both rat NAV1.7 and rat NAV1.8. In a further embodiment, the anti-NAV1.7 and NAV1.8 cross-reactive antibodies are also cross-reactive with both cyno NAV1.7 and cyno NAV1.8. In a further embodiment, the anti-NAV1.7 and NAV1.8 cross-reactive antibodies are also cross-reactive with both mouse NAV1.7 and mouse NAV1.8. In a further embodiment, the anti-NAV1.7 and NAV1.8 cross-reactive antibodies are also cross-reactive with each of rat NAV1.7, rat NAV1.8, mouse NAV1.7 and mouse NAV1.8. In a further embodiment, the anti-NAV1.7 and NAV1.8 cross-reactive antibodies are also cross-reactive with each of rat NAV1.7, rat NAV1.8, cyno NAV1.7 and cyno NAV1.8. In a further embodiment, the anti-NAV1.7 and NAV1.8 cross-reactive antibodies are also cross-reactive with each of mouse NAV1.7, mouse NAV1.8, cyno NAV1.7 and cyno NAV1.8. In a further embodiment, the anti-NAV1.7 and NAV1.8 cross-reactive antibodies are also cross-reactive with three of: rat NAV1.7, cyno NAV1.7, mouse NAV1.7, rat NAV1.8, cyno NAV1.8 and mouse NAV1.8. In a further embodiment, the anti-NAV1.7 and NAV1.8 cross-reactive antibodies are also cross-reactive with four of: rat NAV1.7, cyno NAV1.7, mouse NAV1.7, rat NAV1.8, cyno NAV1.8 and mouse NAV1.8. In a further embodiment, the anti-NAV1.7 and NAV1.8 cross-reactive antibodies are also cross-reactive with five of: rat NAV1.7, cyno NAV1.7, mouse NAV1.7, rat NAV1.8, cyno NAV1.8 and mouse NAV1.8. In a further embodiment, the anti-NAV1.7 and NAV1.8 cross-reactive antibodies are also cross-reactive with each of rat NAV1.7, rat NAV1.8, mouse NAV1.7, mouse NAV1.8, cyno NAV1.7 and cyno NAV1.8. In a further embodiment, the antibodies are also cross-reactive with the corresponding loop sequences in rat NAV1.7, rat NAV1.8, mouse NAV1.7, mouse NAV1.8, cyno NAV1.7 and/or cyno NAV1.8 in any of the preceding embodiments.

### Antibodies to NAV1.8 and NAV1.9

In a fifth embodiment there is provided an antibody which binds to NAV1.8 and NAV1.9, particularly to human NAV1.8 and human NAV1.9. Certain anti-NAV1.8 and NAV1.9 cross-reactive antibodies may bind to conformational epitopes, such as epitopes found on two external loops. Hence, one anti-NAV1.8 and NAV1.9 cross-reactive antibody according to the invention may be defined according to sentence 53 or sentence 54:

Sentence 53. An antibody or fragment thereof which binds to human NAV1.9 (SEQ ID No:38), and wherein the antibody or fragment binds to:
a. A loop selected from the D1E2 loop (SEQ ID No:40), the D2E2 loop (SEQ ID No:44), the D3E2 loop (SEQ ID No:48) and the D4E2 loop (SEQ ID No:52); and
b. One, two or more (e.g. 3, 4, 5 or 6) of the D1E1 loop (SEQ ID No:39), the D1E3 loop (SEQ ID No:41), the D2E1 loop (SEQ ID No:43), the D2E3 loop (SEQ ID No:45), the D3E1 loop (SEQ ID No:47), the D3E3 loop (SEQ ID No:49), the D4E1 loop (SEQ ID No:51) and the D4E3 loop (SEQ ID No:53); and
   which binds to human NAV1.8 (SEQ ID No:20), and wherein the antibody or fragment binds to:
c. A loop selected from the D1E2 loop (SEQ ID No:22), the D2E2 loop (SEQ ID No:26), the D3E2 loop (SEQ ID No:30) and the D4E2 loop (SEQ ID No:34); and
d. One two or more (e.g. 3, 4, 5 or 6) of the D1E1 loop (SEQ ID No:21), the D1E3 loop (SEQ ID No:23), the D2E1 loop (SEQ ID No:25), the D2E3 loop (SEQ ID No:27), the D3E1 loop (SEQ ID No:29), the D3E3 loop (SEQ ID No:31), the D4E1 loop (SEQ ID No:33) and the D4E3 loop (SEQ ID No:35).

Sentence 54. An antibody or fragment according to sentence 53, which binds to human NAV1.9 (SEQ ID No:38), and wherein the antibody or fragment binds to:
a. The D1E2 loop (SEQ ID No:40); and one or both of the D1E1 loop (SEQ ID No:39) and the D1E3 loop (SEQ ID No:41); or
b. the D2E2 loop (SEQ ID No:44), and one or both of the D2E1 loop (SEQ ID No:43) and the D2E3 loop (SEQ ID No:45); or
c. the D3E2 loop (SEQ ID No:48), and one or both of the D3E1 loop (SEQ ID No:47) and the D3E3 loop (SEQ ID No:49); or
d. the D4E2 loop (SEQ ID No:52), and one or both of the D4E1 loop (SEQ ID No:51) and the D4E3 loop (SEQ ID No:53); and
   which binds to human NAV1.8 (SEQ ID No:20), and wherein the antibody or fragment binds to:
e. the D1E2 loop (SEQ ID No:22); and one or both of the D1E1 loop (SEQ ID No:21) and the D1E3 loop (SEQ ID No:23); or
f. the D2E2 loop (SEQ ID No:26), and one or both of the D2E1 loop (SEQ ID No:25) and the D2E3 loop (SEQ ID No:27); or
g. the D3E2 loop (SEQ ID No:30), and one or both of the D3E1 loop (SEQ ID No:29) and the D3E3 loop (SEQ ID No:31); or
h. the D4E2 loop (SEQ ID No:34), and one or both of the D4E1 loop (SEQ ID No:33) and the D4E3 loop (SEQ ID No:35).

These anti-NAV1.8 and NAV1.9 cross-reactive antibodies may be cross-reactive with NAV1.7 proteins, e.g. human NAV1.7 proteins. In a further embodiment, the antibodies cross react with the corresponding loop sequences in NAV1.7 (see Table 1 hereinabove for SEQ ID NOs for human NAV1.7) in any of the preceding embodiments.

Selectivity over other NAV proteins, in particular NAV1.1 to NAV1.6, may provide further benefits in terms of improved treatment of pain diseases or conditions. In another embodiment, selectivity over NAV1.7 may be beneficial. Thus, in one embodiment, the anti-NAV1.8 and NAV1.9 cross-reactive antibodies are selective over any one of NAV1.1, NAV1.2, NAV1.3, NAV1.4, NAV1.5, NAV1.6 and NAV1.7, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.8 and NAV1.9 cross-reactive antibodies are selective over NAV1.4 and NAV1.5. In a further embodiment, the anti-NAV1.8 and NAV1.9 cross-reactive antibodies are selective over NAV1.4 and NAV1.6. In a further embodiment, the anti-NAV1.8 and NAV1.9 cross-reactive antibodies are selective over NAV1.5 and NAV1.6. In a further embodiment, the anti-NAV1.8 and NAV1.9 cross-reactive antibodies are selective over NAV1.4, NAV1.5 and NAV1.6.

In one aspect, the anti-NAV1.8 and NAV1.9 cross-reactive antibodies are selective over at least two other NAV proteins. Thus, in a further embodiment, the anti-NAV1.8 and NAV1.9 cross-reactive antibodies are selective over NAV1.1 and NAV1.2. In a further embodiment, the anti-NAV1.8 and NAV1.9 cross-reactive antibodies are selective over NAV1.1 and NAV1.3. In a further embodiment, the anti-NAV1.8 and NAV1.9 cross-reactive antibodies are selective over NAV1.1 and NAV1.4. In a further embodiment, the anti-NAV1.8 and NAV1.9 cross-reactive antibodies are selective over NAV1.1 and NAV1.5. In a further embodiment, the anti-NAV1.8 and NAV1.9 cross-reactive antibodies are selective over NAV1.1 and NAV1.6. In a further embodiment, the anti-NAV1.8 and NAV1.9 cross-reactive antibodies are selective over NAV1.2 and NAV1.3. In a further embodiment, the anti-NAV1.8 and NAV1.9 cross-reactive antibodies are selective over NAV1.2 and NAV1.4. In a further embodiment, the anti-NAV1.8 and NAV1.9 cross-reactive antibodies are selective over NAV1.2 and NAV1.5. In a further embodiment, the anti-NAV1.8 and NAV1.9 cross-reactive antibodies are selective over NAV1.2 and NAV1.6. In a further embodiment, the anti-NAV1.8 and NAV1.9 cross-reactive antibodies are selective over NAV1.3 and NAV1.4. In a further embodiment, the anti-NAV1.8 and NAV1.9 cross-reactive antibodies are selective over NAV1.3 and NAV1.5. In a further embodiment, the anti-NAV1.8 and NAV1.9 cross-reactive antibodies are selective over NAV1.3 and NAV1.6.

In one aspect, the anti-NAV1.8 and NAV1.9 cross-reactive antibodies are selective over at least three other NAV proteins. Thus, in a further embodiment, the anti-NAV1.8 and NAV1.9 cross-reactive antibodies are selective over NAV1.1, NAV1.2 and NAV1.3. In a further embodiment, the anti-NAV1.8 and NAV1.9 cross-reactive antibodies are selective over NAV1.1, NAV1.2 and NAV1.4. In a further embodiment, the anti-NAV1.8 and NAV1.9 cross-reactive antibodies are selective over NAV1.1, NAV1.2 and NAV1.5. In a further embodiment, the anti-NAV1.8 and NAV1.9 cross-reactive antibodies are selective over NAV1.1, NAV1.2 and NAV1.6. In a further embodiment, the anti-NAV1.8 and NAV1.9 cross-reactive antibodies are selective over NAV1.1, NAV1.3 and NAV1.4. In a further embodiment, the anti-NAV1.8 and NAV1.9 cross-reactive antibodies are selective over NAV1.1, NAV1.3 and NAV1.5. In a further embodiment, the anti-NAV1.8 and NAV1.9 cross-reactive antibodies are selective over NAV1.1, NAV1.3 and NAV1.6. In a further embodiment, the anti-NAV1.8 and NAV1.9 cross-reactive antibodies are selective over NAV1.1, NAV1.4 and NAV1.5. In a further embodiment, the anti-NAV1.8 and NAV1.9 cross-reactive antibodies are selective over NAV1.1, NAV1.4 and NAV1.6. In a further embodiment, the anti-NAV1.8 and NAV1.9 cross-reactive antibodies are selective over NAV1.2, NAV1.3 and NAV1.4. In a further embodiment, the anti-NAV1.8 and NAV1.9 cross-reactive antibodies are selective over NAV1.2, NAV1.3 and NAV1.5. In a further embodiment, the anti-NAV1.8 and NAV1.9 cross-reactive antibodies are selective over NAV1.2, NAV1.3 and NAV1.6. In a further embodiment, the anti-NAV1.8 and NAV1.9 cross-reactive antibodies are selective over NAV1.3, NAV1.4 and NAV1.5. In a further embodiment, the anti-NAV1.8 and NAV1.9 cross-reactive antibodies are selective over NAV1.3, NAV1.4 and NAV1.6.

In a further embodiment, the anti-NAV1.8 and NAV1.9 cross-reactive antibodies are selective over all of NAV1.1, NAV1.2, NAV1.3, NAV1.4, NAV1.5 and NAV1.6.

In order to be useful in in vivo models of pain, it is advantageous for the anti-NAV1.8 and NAV1.9 cross-reactive antibodies as disclosed herein to be additionally cross-reactive with NAV1.8 and NAV1.9 proteins from other species, in particular rat, mouse and monkey (e.g. cynomolgus). Thus, in one embodiment, the anti-NAV1.8 and NAV1.9 cross-reactive antibodies are also cross-reactive with rat NAV1.8 (SEQ ID No:165). In another embodiment, the anti-NAV1.8 and NAV1.9 cross-reactive antibodies are also cross-reactive mouse NAV1.8 (SEQ ID No:78). In another embodiment, the anti-NAV1.8 and NAV1.9 cross-reactive antibodies are also cross-reactive with cyno NAV1.8 (SEQ ID No:166). In another embodiment, the anti-NAV1.8 and NAV1.9 cross-reactive antibodies are also cross-reactive with rat NAV1.9 (SEQ ID No:167). In another embodiment, the anti-NAV1.8 and NAV1.9 cross-reactive antibodies are also cross-reactive with cyno NAV1.9 (SEQ ID No:168). In another embodiment, the anti-NAV1.8 and NAV1.9 cross-reactive antibodies are also cross-reactive with mouse NAV1.9 (SEQ ID No:82). In a further embodiment, the anti-NAV1.8 and NAV1.9 cross-reactive antibodies are also cross-reactive with both rat NAV1.8 and rat NAV1.9. In a further embodiment, the anti-NAV1.8 and NAV1.9 cross-reactive antibodies are also cross-reactive with both cyno NAV1.8 and cyno NAV1.9. In a further embodiment, the anti-NAV1.8 and NAV1.9 cross-reactive antibodies are also cross-reactive with both mouse NAV1.8 and mouse NAV1.9. In a further embodiment, the anti-NAV1.8 and NAV1.9 cross-reactive antibodies are also cross-reactive with each of rat NAV1.8, rat NAV1.9, mouse NAV1.8 and mouse NAV1.9. In a further embodiment, the anti-NAV1.8 and NAV1.9 cross-reactive antibodies are also cross-reactive with each of rat NAV1.8, rat NAV1.9, cyno NAV1.8 and cyno NAV1.9. In a further embodiment, the anti-NAV1.8 and NAV1.9 cross-reactive antibodies are also cross-reactive with each of mouse NAV1.8, mouse NAV1.9, cyno NAV1.8 and cyno NAV1.9. In a further embodiment, the anti-NAV1.8 and NAV1.9 cross-reactive antibodies are also cross-reactive with three of: rat NAV1.8, cyno NAV1.8, mouse NAV1.8, rat NAV1.9, cyno NAV1.9 and mouse NAV1.9. In a further embodiment, the anti-NAV1.8 and NAV1.9 cross-reactive antibodies are also cross-reactive with four of: rat NAV1.8, cyno NAV1.8, mouse NAV1.8, rat NAV1.9, cyno NAV1.9 and mouse NAV1.9. In a further embodiment, the anti-NAV1.8 and NAV1.9 cross-reactive antibodies are also cross-reactive with five of: rat NAV1.8, cyno NAV1.8, mouse NAV1.8, rat NAV1.9, cyno NAV1.9 and mouse NAV1.9. In a further embodiment, the anti-NAV1.8 and NAV1.9 cross-reactive antibodies are also cross-reactive with each of rat NAV1.8, rat NAV1.9, mouse NAV1.8, mouse NAV1.9, cyno NAV1.8 and cyno NAV1.9. In a further embodiment, the antibodies are also cross-reactive with the corresponding loop sequences in rat NAV1.8, rat NAV1.9, mouse NAV1.8, mouse NAV1.9, cyno NAV1.8 and/or cyno NAV1.9 in any of the preceding embodiments.

### Antibodies to NAV1.7 and NAV1.9

In a sixth embodiment there is provided an antibody which binds to NAV1.7 and NAV1.9, particularly to human NAV1.7 and human NAV1.9. Certain anti-NAV1.7 and NAV1.9 cross-reactive antibodies may bind to conformational epitopes, such as epitopes found on two external loops. Hence, one anti-NAV1.7 and NAV1.9 cross-reactive antibody according to the invention may be defined according to sentence 66 or sentence 67:

Sentence 66. An antibody or fragment thereof which binds to human NAV1.7 (SEQ ID No:2), and wherein the antibody or fragment binds to:
a. A loop selected from the D1E2 loop (SEQ ID No:4), the D2E2 loop (SEQ ID No:8), the D3E2 loop (SEQ ID No:12) and the D4E2 loop (SEQ ID No:16); and
b. One, two or more (e.g. 3, 4, 5 or 6) of the D1E1 loop (SEQ ID No:3), the D1E3 loop (SEQ ID No:5), the D2E1 loop (SEQ ID No:7), the D2E3 loop (SEQ ID No:9), the D3E1 loop (SEQ ID No:11), the D3E3 loop (SEQ ID No:13), the D4E1 loop (SEQ ID No:15) and the D4E3 loop (SEQ ID No:17); and
   which binds to human NAV1.9 (SEQ ID No:38), and wherein the antibody or fragment binds to:
c. A loop selected from the D1E2 loop (SEQ ID No:40), the D2E2 loop (SEQ ID No:44), the D3E2 loop (SEQ ID No:48) and the D4E2 loop (SEQ ID No:52); and
d. One two or more (e.g. 3, 4, 5 or 6) of the D1E1 loop (SEQ ID No:39), the D1E3 loop (SEQ ID No:41), the D2E1 loop (SEQ ID No:43), the D2E3 loop (SEQ ID No:45), the the D3E1 loop (SEQ ID No:47), the D3E3 loop (SEQ ID No:49), the D4E1 loop (SEQ ID No:51) and the D4E3 loop (SEQ ID No:53).

Sentence 67. An antibody or fragment according to sentence 66, which binds to human NAV1.7 (SEQ ID No:2), and wherein the antibody or fragment binds to:
a. The D1E2 loop (SEQ ID No:4); and one or both of the D1E1 loop (SEQ ID No:3) and the D1E3 loop (SEQ ID No:5); or
b. the D2E2 loop (SEQ ID No:8), and one or both of the D2E1 loop (SEQ ID No:7) and the D2E3 loop (SEQ ID No:9); or
c. the D3E2 loop (SEQ ID No:12), and one or both of the D3E1 loop (SEQ ID No:11) and the D3E3 loop (SEQ ID No:13); or
d. the D4E2 loop (SEQ ID No:16), and one or both of the D4E1 loop (SEQ ID No:15) and the D4E3 loop (SEQ ID No:17); and
   which binds to human NAV1.9 (SEQ ID No:38), and wherein the antibody or fragment binds to:
e. the D1E2 loop (SEQ ID No:40); and one or both of the D1E1 loop (SEQ ID No:39) and the D1E3 loop (SEQ ID No:41); or
f. the D2E2 loop (SEQ ID No:44), and one or both of the D2E1 loop (SEQ ID No:43) and the D2E3 loop (SEQ ID No:45); or
g. the D3E2 loop (SEQ ID No:48), and one or both of the D3E1 loop (SEQ ID No:47) and the D3E3 loop (SEQ ID No:49); or
h. the D4E2 loop (SEQ ID No:52), and one or both of the D4E1 loop (SEQ ID No:51) and the D4E3 loop (SEQ ID No:53).

These anti-NAV1.7 and NAV1.9 cross-reactive antibodies may be cross-reactive with NAV1.8 proteins, e.g. human NAV1.8 proteins. In a further embodiment, the antibodies cross react with the corresponding loop sequences in NAV1.8 (see Table 1 hereinabove for SEQ ID NOs for human NAV1.8) in any of the preceding embodiments.

Selectivity over other NAV proteins, in particular NAV1.1 to NAV1.6, may provide further benefits in terms of improved treatment of pain diseases or conditions. In another embodiment, selectivity over NAV1.8 may be beneficial. Thus, in one embodiment, the anti-NAV1.7 and NAV1.9 cross-reactive antibodies are selective over any one of NAV1.1, NAV1.2, NAV1.3, NAV1.4, NAV1.5, NAV1.6 and NAV1.8, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.7 and NAV1.9 cross-reactive antibodies are selective over NAV1.4 and NAV1.5. In a further embodiment, the anti-NAV1.7 and NAV1.9 cross-reactive antibodies are selective over NAV1.4 and NAV1.6. In a further embodiment, the anti-NAV1.7 and NAV1.9 cross-reactive antibodies are selective over NAV1.5 and NAV1.6. In a further embodiment, the anti-NAV1.7 and NAV1.9 cross-reactive antibodies are selective over NAV1.4, NAV1.5 and NAV1.6.

In one aspect, the anti-NAV1.7 and NAV1.9 cross-reactive antibodies are selective over at least two other NAV proteins. Thus, in a further embodiment, the anti-NAV1.7 and NAV1.9 cross-reactive antibodies are selective over NAV1.1 and NAV1.2. In a further embodiment, the anti-NAV1.7 and NAV1.9 cross-reactive antibodies are selective over NAV1.1 and NAV1.3. In a further embodiment, the anti-NAV1.7 and NAV1.9 cross-reactive antibodies are selective over NAV1.1 and NAV1.4. In a further embodiment, the anti-NAV1.7 and NAV1.9 cross-reactive antibodies are selective over NAV1.1 and NAV1.5. In a further embodiment, the anti-NAV1.7 and NAV1.9 cross-reactive antibodies are selective over NAV1.1 and NAV1.6. In a further embodiment, the anti-NAV1.7 and NAV1.9 cross-reactive antibodies are selective over NAV1.2 and NAV1.3. In a further embodiment, the anti-NAV1.7 and NAV1.9 cross-reactive antibodies are selective over NAV1.2 and NAV1.4. In a further embodiment, the anti-NAV1.7 and NAV1.9 cross-reactive antibodies are selective over NAV1.2 and NAV1.5. In a further embodiment, the anti-NAV1.7 and NAV1.9 cross-reactive antibodies are selective over NAV1.2 and NAV1.6. In a further embodiment, the anti-NAV1.7 and NAV1.9 cross-reactive antibodies are selective over NAV1.3 and NAV1.4. In a further embodiment, the anti-NAV1.7 and NAV1.9 cross-reactive antibodies are selective over NAV1.3 and NAV1.5. In a further embodiment, the anti-NAV1.7 and NAV1.9 cross-reactive antibodies are selective over NAV1.3 and NAV1.6.

In one aspect, the anti-NAV1.7 and NAV1.9 cross-reactive antibodies are selective over at least three other NAV proteins. Thus, in a further embodiment, the anti-NAV1.7 and NAV1.9 cross-reactive antibodies are selective over NAV1.1, NAV1.2 and NAV1.3. In a further embodiment, the anti-NAV1.7 and NAV1.9 cross-reactive antibodies are selective over NAV1.1, NAV1.2 and NAV1.4. In a further embodiment, the anti-NAV1.7 and NAV1.9 cross-reactive antibodies are selective over NAV1.1, NAV1.2 and NAV1.5. In a further embodiment, the anti-NAV1.7 and NAV1.9 cross-reactive antibodies are selective over NAV1.1, NAV1.2 and NAV1.6. In a further embodiment, the anti-NAV1.7 and NAV1.9 cross-reactive antibodies are selective over NAV1.1, NAV1.3 and NAV1.4. In a further embodiment, the anti-NAV1.7 and NAV1.9 cross-reactive antibodies are selective over NAV1.1, NAV1.3 and NAV1.5. In a further embodiment, the anti-NAV1.7 and NAV1.9 cross-reactive antibodies are selective over NAV1.1, NAV1.3 and NAV1.6. In a further embodiment, the anti-NAV1.7 and NAV1.9 cross-reactive antibodies are selective over NAV1.1, NAV1.4 and NAV1.5. In a further embodiment, the anti-NAV1.7 and NAV1.9 cross-reactive antibodies are selective over NAV1.1, NAV1.4 and NAV1.6. In a further embodiment, the anti-NAV1.7 and NAV1.9 cross-reactive antibodies are selective over NAV1.2, NAV1.3 and NAV1.4. In a further embodiment, the anti-NAV1.7 and NAV1.9 cross-reactive antibodies are selective over NAV1.2, NAV1.3 and NAV1.5. In a further embodiment, the anti-NAV1.7 and NAV1.9 cross-reactive antibodies are selective over NAV1.2, NAV1.3 and NAV1.6. In a further embodiment, the anti-NAV1.7 and NAV1.9 cross-reactive antibodies are selective over NAV1.3, NAV1.4 and NAV1.5. In a further embodiment, the anti-NAV1.7 and NAV1.9 cross-reactive antibodies are selective over NAV1.3, NAV1.4 and NAV1.6.

In a further embodiment, the anti-NAV1.7 and NAV1.9 cross-reactive antibodies are selective over all of NAV1.1, NAV1.2, NAV1.3, NAV1.4, NAV1.5 and NAV1.6.

In order to be useful in in vivo models of pain, it is advantageous for the anti-NAV1.7 and NAV1.9 cross-reactive antibodies as disclosed herein to be additionally cross-reactive with NAV1.7 and NAV1.9 proteins from other species, in particular rat, mouse and monkey (e.g. cynomolgus). Thus, in one embodiment, the anti-NAV1.7 and NAV1.9 cross-reactive antibodies are also cross-reactive with rat NAV1.7 (SEQ ID No:163). In another embodiment, the anti-NAV1.7 and NAV1.9 cross-reactive antibodies are also cross-reactive mouse NAV1.7 (SEQ ID No:72). In another embodiment, the anti-NAV1.7 and NAV1.9 cross-reactive antibodies are also cross-reactive with cyno NAV1.7 (SEQ ID No:164). In another embodiment, the anti-NAV1.7 and NAV1.9 cross-reactive antibodies are also cross-reactive with rat NAV1.9 (SEQ ID No:167). In another embodiment, the anti-NAV1.7 and NAV1.9 cross-reactive antibodies are also cross-reactive with cyno NAV1.9 (SEQ ID No:168). In another embodiment, the anti-NAV1.7 and NAV1.9 cross-reactive antibodies are also cross-reactive with mouse NAV1.9 (SEQ ID No:82). In a further embodiment, the anti-NAV1.7 and NAV1.9 cross-reactive antibodies are also cross-reactive with both rat NAV1.7 and rat NAV1.9. In a further embodiment, the anti-NAV1.7 and NAV1.9 cross-reactive antibodies are also cross-reactive with both cyno NAV1.7 and cyno NAV1.9. In a further embodiment, the anti-NAV1.7 and NAV1.9 cross-reactive antibodies are also cross-reactive with both mouse NAV1.7 and mouse NAV1.9. In a further embodiment, the anti-NAV1.7 and NAV1.9 cross-reactive antibodies are also cross-reactive with each of rat NAV1.7, rat NAV1.9, mouse NAV1.7 and mouse NAV1.9. In a further embodiment, the anti-NAV1.7 and NAV1.9 cross-reactive antibodies are also cross-reactive with each of rat NAV1.7, rat NAV1.9, cyno NAV1.7 and cyno NAV1.9. In a further embodiment, the anti-NAV1.7 and NAV1.9 cross-reactive antibodies are also cross-reactive with each of mouse NAV1.7, mouse NAV1.9, cyno NAV1.7 and cyno NAV1.9. In a further embodiment, the anti-NAV1.7 and NAV1.9 cross-reactive antibodies are also cross-reactive with each of rat NAV1.7, rat NAV1.9, mouse NAV1.7, mouse NAV1.9, cyno NAV1.7 and cyno NAV1.9. In a further embodiment, the anti-NAV1.7 and NAV1.9 cross-reactive antibodies are also cross-reactive with three of: rat NAV1.7, cyno NAV1.7, mouse NAV1.7, rat NAV1.9, cyno NAV1.9 and mouse NAV1.9. In a further embodiment, the anti-NAV1.7 and NAV1.9 cross-reactive antibodies are also cross-reactive with four of: rat NAV1.7, cyno NAV1.7, mouse NAV1.7, rat NAV1.9, cyno NAV1.9 and mouse NAV1.9. In a further embodiment, the anti-NAV1.7 and NAV1.9 cross-reactive antibodies are also cross-reactive with five of: rat NAV1.7, cyno NAV1.7, mouse NAV1.7, rat NAV1.9, cyno NAV1.9 and mouse NAV1.9. In a further embodiment, the antibodies are also cross-reactive with the corresponding loop sequences in rat NAV1.7, rat NAV1.9, mouse NAV1.7, mouse NAV1.9, cyno NAV1.7 and/or cyno NAV1.9 in any of the preceding embodiments.

### Antibodies to NAV1.7, NAV1.8 and NAV1.9

In a seventh embodiment there is provided an antibody which binds to each of NAV1.7, NAV1.8 and NAV1.9, particularly to human NAV1.7, human NAV1.8 and human NAV1.9. Certain anti-NAV1.7, NAV1.8 and NAV1.9 cross-reactive antibodies may bind to conformational epitopes, such as epitopes found on two external loops. Hence, one anti-NAV1.7, NAV1.8 and NAV1.9 cross-reactive antibody according to the invention may be defined according to sentence 79 or sentence 80:

Sentence 79. An antibody or fragment thereof which binds to human NAV1.7 (SEQ ID No:2), and wherein the antibody or fragment binds to:
a. A loop selected from the D1E2 loop (SEQ ID No:4), the D2E2 loop (SEQ ID No:8), the D3E2 loop (SEQ ID No:12) and the D4E2 loop (SEQ ID No:16); and
b. One, two or more (e.g. 3, 4, 5 or 6) of the D1E1 loop (SEQ ID No:3), the D1E3 loop (SEQ ID No:5), the D2E1 loop (SEQ ID No:7), the D2E3 loop (SEQ ID No:9), the D3E1 loop (SEQ ID No:11), the D3E3 loop (SEQ ID No:13), the D4E1 loop (SEQ ID No:15) and the D4E3 loop (SEQ ID No:17); and
   which binds to human NAV1.8 (SEQ ID No:20), and wherein the antibody or fragment binds to:
c. A loop selected from the D1E2 loop (SEQ ID No:22), the D2E2 loop (SEQ ID No:26), the D3E2 loop (SEQ ID No:30) and the D4E2 loop (SEQ ID No:34); and
d. One, two or more (e.g. 3, 4, 5 or 6) of the D1E1 loop (SEQ ID No:21), the D1E3 loop (SEQ ID No:23), the D2E1 loop (SEQ ID No:25), the D2E3 loop (SEQ ID No:27), the D3E1 loop (SEQ ID No:29), the D3E3 loop (SEQ ID No:31), the D4E1 loop (SEQ ID No:33) and the D4E3 loop (SEQ ID No:35); and
   which binds to human NAV1.9 (SEQ ID No:38), and wherein the antibody or fragment binds to:
e. A loop selected from the D1E2 loop (SEQ ID No:40), the D2E2 loop (SEQ ID No:44), the D3E2 loop (SEQ ID No:48) and the D4E2 loop (SEQ ID No:52); and
f. One two or more (e.g. 3, 4, 5 or 6) of the D1E1 loop (SEQ ID No:39), the D1E3 loop (SEQ ID No:41), the D2E1 loop (SEQ ID No:43), the D2E3 loop (SEQ ID No:45), the D3E1 loop (SEQ ID No:47), the D3E3 loop (SEQ ID No:49), the D4E1 loop (SEQ ID No:51) and the D4E3 loop (SEQ ID No:53).

Sentence 80. An antibody or fragment according to sentence 79, which binds to human NAV1.7 (SEQ ID No:2), and wherein the antibody or fragment binds to:
a. The D1E2 loop (SEQ ID No:4); and one or both of the D1E1 loop (SEQ ID No:3) and the D1E3 loop (SEQ ID No:5); or
b. the D2E2 loop (SEQ ID No:8), and one or both of the D2E1 loop (SEQ ID No:7) and the D2E3 loop (SEQ ID No:9); or
c. the D3E2 loop (SEQ ID No:12), and one or both of the D3E1 loop (SEQ ID No:11) and the D3E3 loop (SEQ ID No:13); or
d. the D4E2 loop (SEQ ID No:16), and one or both of the D4E1 loop (SEQ ID No:15) and the D4E3 loop (SEQ ID No:17); and
   which binds to human NAV1.8 (SEQ ID No:20), and wherein the antibody or fragment binds to:
e. the D1E2 loop (SEQ ID No:22); and one or both of the D1E1 loop (SEQ ID No:21) and the D1E3 loop (SEQ ID No:23); or
f. the D2E2 loop (SEQ ID No:26), and one or both of the D2E1 loop (SEQ ID No:25) and the D2E3 loop (SEQ ID No:27); or
g. the D3E2 loop (SEQ ID No:30), and one or both of the D3E1 loop (SEQ ID No:29) and the D3E3 loop (SEQ ID No:31); or
h. the D4E2 loop (SEQ ID No:34), and one or both of the D4E1 loop (SEQ ID No:33) and the D4E3 loop (SEQ ID No:35); and
   which binds to human NAV1.9 (SEQ ID No:38), and wherein the antibody or fragment binds to:
e. the D1E2 loop (SEQ ID No:40); and one or both of the D1E1 loop (SEQ ID No:39) and the D1E3 loop (SEQ ID No:41); or
f. the D2E2 loop (SEQ ID No:44), and one or both of the D2E1 loop (SEQ ID No:43) and the D2E3 loop (SEQ ID No:45); or
g. the D3E2 loop (SEQ ID No:48), and one or both of the D3E1 loop (SEQ ID No:47) and the D3E3 loop (SEQ ID No:49); or
h. the D4E2 loop (SEQ ID No:52), and one or both of the D4E1 loop (SEQ ID No:51) and the D4E3 loop (SEQ ID No:53).

Selectivity over other NAV proteins, in particular NAV1.1 to NAV1.6, may provide further benefits in terms of improved treatment of pain diseases or conditions. Thus, in one embodiment, the anti-NAV1.7, NAV1.8 and NAV1.9 cross-reactive antibodies are selective over any one of NAV1.1, NAV1.2, NAV1.3, NAV1.4, NAV1.5 and NAV1.6, e.g. are selective over NAV1.6.

Because of their role in muscle contraction and motor neuron activity, selectivity over NAV1.4, NAV1.5 and/or NAV1.6 may provide antibodies more suited for the treatment of certain types of pain. Thus, in another embodiment, the anti-NAV1.7 and NAV1.9 cross-reactive antibodies are selective over NAV1.4 and NAV1.5. In a further embodiment, the anti-NAV1.7, NAV1.8 and NAV1.9 cross-reactive antibodies are selective over NAV1.4 and NAV1.6. In a further embodiment, the anti-NAV1.7, NAV1.8 and NAV1.9 cross-reactive antibodies are selective over NAV1.5 and NAV1.6. In a further embodiment, the anti-NAV1.7, NAV1.8 and NAV1.9 cross-reactive antibodies are selective over NAV1.4, NAV1.5 and NAV1.6.

In one aspect, the anti-NAV1.7, NAV1.8 and NAV1.9 cross-reactive antibodies are selective over at least two other NAV proteins. Thus, in a further embodiment, the anti-NAV1.7, NAV1.8 and NAV1.9 cross-reactive antibodies are selective over NAV1.1 and NAV1.2. In a further embodiment, the anti-NAV1.7, NAV1.8 and NAV1.9 cross-reactive antibodies are selective over NAV1.1 and NAV1.3. In a further embodiment, the anti-NAV1.7, NAV1.8 and NAV1.9 cross-reactive antibodies are selective over NAV1.1 and NAV1.4. In a further embodiment, the anti-NAV1.7, NAV1.8 and NAV1.9 cross-reactive antibodies are selective over NAV1.1 and NAV1.5. In a further embodiment, the anti-NAV1.7, NAV1.8 and NAV1.9 cross-reactive antibodies are selective over NAV1.1 and NAV1.6. In a further embodiment, the anti-NAV1.7, NAV1.8 and NAV1.9 cross-reactive antibodies are selective over NAV1.2 and NAV1.3. In a further embodiment, the anti-NAV1.7, NAV1.8 and NAV1.9 cross-reactive antibodies are selective over NAV1.2 and NAV1.4. In a further embodiment, the anti-NAV1.7, NAV1.8 and NAV1.9 cross-reactive antibodies are selective over NAV1.2 and NAV1.5. In a further embodiment, the anti-NAV1.7, NAV1.8 and NAV1.9 cross-reactive antibodies are selective over NAV1.2 and NAV1.6. In a further embodiment, the anti-NAV1.7, NAV1.8 and NAV1.9 cross-reactive antibodies are selective over NAV1.3 and NAV1.4. In a further embodiment, the anti-NAV1.7, NAV1.8 and NAV1.9 cross-reactive antibodies are selective over NAV1.3 and NAV1.5. In a further embodiment, the anti-NAV1.7, NAV1.8 and NAV1.9 cross-reactive antibodies are selective over NAV1.3 and NAV1.6.

In one aspect, the anti-NAV1.7, NAV1.8 and NAV1.9 cross-reactive antibodies are selective over at least three other NAV proteins. Thus, in a further embodiment, the anti-NAV1.7, NAV1.8 and NAV1.9 cross-reactive antibodies are selective over NAV1.1, NAV1.2 and NAV1.3. In a further embodiment, the anti-NAV1.7, NAV1.8 and NAV1.9 cross-reactive antibodies are selective over NAV1.1, NAV1.2 and NAV1.4. In a further embodiment, the anti-NAV1.7, NAV1.8 and NAV1.9 cross-reactive antibodies are selective over NAV1.1, NAV1.2 and NAV1.5. In a further embodiment, the anti-NAV1.7, NAV1.8 and NAV1.9 cross-reactive antibodies are selective over NAV1.1, NAV1.2 and NAV1.6. In a further embodiment, the anti-NAV1.7, NAV1.8 and NAV1.9 cross-reactive antibodies are selective over NAV1.1, NAV1.3 and NAV1.4. In a further embodiment, the anti-NAV1.7, NAV1.8 and NAV1.9 cross-reactive antibodies are selective over NAV1.1, NAV1.3 and NAV1.5. In a further embodiment, the anti-NAV1.7, NAV1.8 and NAV1.9 cross-reactive antibodies are selective over NAV1.1, NAV1.3 and NAV1.6. In a further embodiment, the anti-NAV1.7, NAV1.8 and NAV1.9 cross-reactive antibodies are selective over NAV1.1, NAV1.4 and NAV1.5. In a further embodiment, the anti-NAV1.7, NAV1.8 and NAV1.9 cross-reactive antibodies are selective over NAV1.1, NAV1.4 and NAV1.6. In a further embodiment, the anti-NAV1.7, NAV1.8 and NAV1.9 cross-reactive antibodies are selective over NAV1.2, NAV1.3 and NAV1.4. In a further embodiment, the anti-NAV1.7, NAV1.8 and NAV1.9 cross-reactive antibodies are selective over NAV1.2, NAV1.3 and NAV1.5. In a further embodiment, the anti-NAV1.7, NAV1.8 and NAV1.9 cross-reactive antibodies are selective over NAV1.2, NAV1.3 and NAV1.6. In a further embodiment, the anti-NAV1.7, NAV1.8 and NAV1.9 cross-reactive antibodies are selective over NAV1.3, NAV1.4 and NAV1.5. In a further embodiment, the anti-NAV1.7, NAV1.8 and NAV1.9 cross-reactive antibodies are selective over NAV1.3, NAV1.4 and NAV1.6.

In a further embodiment, the anti-NAV1.7, NAV1.8 and NAV1.9 cross-reactive antibodies are selective over all of NAV1.1, NAV1.2, NAV1.3, NAV1.4, NAV1.5 and NAV1.6.

In order to be useful in in vivo models of pain, it is advantageous for the anti-NAV1.7, NAV1.8 and NAV1.9 cross-reactive antibodies as disclosed herein to be additionally cross-reactive with NAV1.7, NAV1.8 and NAV1.9 proteins from other species, in particular rat, mouse and monkey (e.g. cynomolgus). Thus, in one embodiment, the anti-NAV1.7, NAV1.8 and NAV1.9 cross-reactive antibodies are also cross-reactive with rat NAV1.7 (SEQ ID No:163). In another embodiment, the anti-NAV1.7, NAV1.8 and NAV1.9 cross-reactive antibodies are also cross-reactive mouse NAV1.7 (SEQ ID No:72). In another embodiment, the anti-NAV1.7, NAV1.8 and NAV1.9 cross-reactive antibodies are also cross-reactive with cyno NAV1.7 (SEQ ID No:164). In another embodiment, the anti-NAV1.7, NAV1.8 and NAV1.9 cross-reactive antibodies are also cross-reactive with rat NAV1.8 (SEQ ID No:165). In another embodiment, the anti-NAV1.7, NAV1.8 and NAV1.9 cross-reactive antibodies are also cross-reactive mouse NAV1.8 (SEQ ID No:78). In another embodiment, the anti-NAV1.7, NAV1.8 and NAV1.9 cross-reactive antibodies are also cross-reactive with cyno NAV1.8 (SEQ ID No:166). In another embodiment, the anti-NAV1.7, NAV1.8 and NAV1.9 cross-reactive antibodies are also cross-reactive with rat NAV1.9 (SEQ ID No:167). In another embodiment, the anti-NAV1.7, NAV1.8 and NAV1.9 cross-reactive antibodies are also cross-reactive with cyno NAV1.9 (SEQ ID No:168). In another embodiment, the anti-NAV1.7, NAV1.8 and NAV1.9 cross-reactive antibodies are also cross-reactive with mouse NAV1.9 (SEQ ID No:82). In a further embodiment, the anti-NAV1.7, NAV1.8 and NAV1.9 cross-reactive antibodies are also cross-reactive with each of rat NAV1.7, rat NAV1.8 and rat NAV1.9. In a further embodiment, the anti-NAV1.7, NAV1.8 and NAV1.9 cross-reactive antibodies are also cross-reactive with each of cyno NAV1.7, cyno NAV1.8 and cyno NAV1.9. In a further embodiment, the anti-NAV1.7, NAV1.8 and NAV1.9 cross-reactive antibodies are also cross-reactive with each of mouse NAV1.7, mouse NAV1.8 and mouse NAV1.9. In a further embodiment, the anti-NAV1.7, NAV1.8 and NAV1.9 cross-reactive antibodies are also cross-reactive with each of rat NAV1.7, rat NAV1.8, rat NAV1.9, mouse NAV1.7, mouse NAV1.8 and mouse NAV1.9. In a further embodiment, the anti-NAV1.7, NAV1.8 and NAV1.9 cross-reactive antibodies are also cross-reactive with each of rat NAV1.7, rat NAV1.8, rat NAV1.9, cyno NAV1.7, cyno NAV1.8 and cyno NAV1.9. In a further embodiment, the anti-NAV1.7, NAV1.8 and NAV1.9 cross-reactive antibodies are also cross-reactive with each of mouse NAV1.7, mouse NAV1.8, mouse NAV1.9, cyno NAV1.7, cyno NAV1.8 and cyno NAV1.9. In a further embodiment, the anti-NAV1.7, NAV1.8 and NAV1.9 cross-reactive antibodies are also cross-reactive with each of rat NAV1.7, rat NAV1.8, rat NAV1.9, mouse NAV1.7, mouse NAV1.8, mouse NAV1.9, cyno NAV1.7, cyno NAV1.8 and cyno NAV1.9. In a further embodiment, the anti-NAV1.7 and NAV1.9 cross-reactive antibodies are also cross-reactive with three of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the anti-NAV1.7 and NAV1.9 cross-reactive antibodies are also cross-reactive with four of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the anti-NAV1.7 and NAV1.9 cross-reactive antibodies are also cross-reactive with five of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the anti-NAV1.7 and NAV1.9 cross-reactive antibodies are also cross-reactive with six of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the anti-NAV1.7 and NAV1.9 cross-reactive antibodies are also cross-reactive with seven of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the anti-NAV1.7 and NAV1.9 cross-reactive antibodies are also cross-reactive with eight of: rat NAV1.7, cyno NAV1.7, mouse NAV1.7, rat NAV1.8, cyno NAV1.8, mouse NAV1.8, rat NAV1.9, cyno NAV1.9 and mouse NAV1.9. In a further embodiment, the antibodies are also cross-reactive with the corresponding loop sequences in rat NAV1.7, rat NAV1.9, mouse NAV1.7, mouse NAV1.9, cyno NAV1.7 and/or cyno NAV1.9 in any of the preceding embodiments.

### Other Selective Antibodies

Other anti-NAV antibodies which are selective over one or more other NAV proteins are described in the sentences below.

Sentence 90. An antibody or fragment thereof which binds to human NAV1.7 (SEQ ID No:2), and comprises one, more (e.g. 2, 3, 4 or 5), or all of the features a. to s.:
a. is selective for a first polypeptide comprising the sequence LTEF (SEQ ID No:169, NAV1.7 motif) over a second polypeptide comprising the sequence ITEF (SEQ ID No:170, NAV1.6 motif);
b. is selective for a first polypeptide comprising the sequence LTEFVNLGN (SEQ ID No:4, NAV1.7 motif) over a second polypeptide comprising the sequence ITEFVNLGN (SEQ ID No:148, NAV1.6 motif);
c. is selective for a first polypeptide comprising the sequence LTEFVN (SEQ ID No:171, NAV1.7 motif) over a second polypeptide comprising the sequence VTEFVD (SEQ ID No:172, NAV1.1, NAV1.2 and NAV1.3 motif);
d. is selective for a first polypeptide comprising the sequence LTEF (SEQ ID No:169, NAV1.7 motif) over a second polypeptide comprising the sequence VTEF (SEQ ID No:240, NAV1.1, NAV1.2 and NAV1.3 motif);
e. is selective for a first polypeptide comprising the sequence LTEFV (SEQ ID No:305, NAV1.7 motif) over a second polypeptide comprising the sequence VTEFV (SEQ ID No:303, NAV1.1, NAV1.2 and NAV1.3 motif);
f. is selective for a first polypeptide comprising the sequence LTEFVNLGN (SEQ ID No:4, NAV1.7 motif) over a second polypeptide comprising the sequence VTEFVDLGN (SEQ ID No:58, NAV1.1, NAV1.2 and NAV1.3 motif);
g. is selective for a first polypeptide comprising the sequence FVNLG (SEQ ID No:173, NAV1.7 motif) over a second polypeptide comprising the sequence FVDLG (SEQ ID No:174, NAV1.1, NAV1.2, NAV1.3, NAV1.4 and NAV1.5 motif);
h. is selective for a first polypeptide comprising the sequence LTEFVN (SEQ ID No:171, NAV1.7 motif) over a second polypeptide comprising the sequence LTEFVD (SEQ ID No:239, NAV1.4 motif);
i. is selective for a first polypeptide comprising the sequence LTEFVNLGN (SEQ ID No:4, NAV1.7 motif) over a second polypeptide comprising the sequence LTEFVDLGN (SEQ ID No:112, NAV1.4 motif);
j. is selective for a first polypeptide comprising the sequence LTEFVN (SEQ ID No:171, NAV1.7 motif) over a second polypeptide comprising the sequence TTEFVD (SEQ ID No:175, NAV1.5 motif);
k. is selective for a first polypeptide comprising the sequence LTEFV (SEQ ID No:305, NAV1.7 motif) over a second polypeptide comprising the sequence TTEFV (SEQ ID No:306, NAV1.5 motif);
l. is selective for a first polypeptide comprising the sequence LTEFVNLGN (SEQ ID No:4, NAV1.7 motif) over a second polypeptide comprising the sequence TTEFVDLGN (SEQ ID No:130, NAV1.5 motif);
m. is selective for a first polypeptide comprising the sequence LTEFV (SEQ ID No:305, NAV1.7 motif) over a second polypeptide comprising the sequence VGTAI (SEQ ID No:307, NAV1.8 motif);
n. is selective for a first polypeptide comprising the sequence EFVNL (SEQ ID No:300, NAV1.7 motif) over a second polypeptide comprising the sequence TAIDL (SEQ ID No:196, NAV1.8 motif);
o. is selective for a first polypeptide comprising the sequence FVNLG (SEQ ID No:173, NAV1.7 motif) over a second polypeptide comprising the sequence IDLRG (SEQ ID No:236, NAV1.8 motif);
p. is selective for a first polypeptide comprising the sequence LTEFVNLGN (SEQ ID No:4, NAV1.7 motif) over a second polypeptide comprising the sequence VGTAIDLRG (SEQ ID No:22, NAV1.8 motif);
q. is selective for a first polypeptide comprising the sequence LTEFV (SEQ ID No:305, NAV1.7 motif) over a second polypeptide comprising the sequence VSYIP (SEQ ID No:298, NAV1.9 motif);
r. is selective for a first polypeptide comprising the sequence VNLGN (SEQ ID No:237, NAV1.7 motif) over a second polypeptide comprising the sequence GITIK (SEQ ID No:302, NAV1.9 motif); and
s. is selective for a first polypeptide comprising the sequence LTEFVNLGN (SEQ ID No:4, NAV1.7 motif) over a second polypeptide comprising the sequence VSYIPGITIK (SEQ ID No:40, NAV1.9 motif).

Sentence 91. An antibody or fragment according to:
a. Sentence 90a or sentence 90b, wherein the second polypeptide is a human NAV1.6 protein; or
b. Sentence 90c, 90d, 90e, 90f or sentence 90g, wherein the second polypeptide is a human NAV1.1 protein, a human NAV1.2 protein or a human NAV1.3 protein; or
c. Sentence 90g, 90h or sentence 90i, wherein the second polypeptide is a human NAV1.4 protein; or
d. Sentence 90g, 90j, 90k or sentence 90l, wherein the second polypeptide is a human NAV1.5 protein; or
e. Sentence 90m, 90n, 900 or sentence 90p, wherein the second polypeptide is a human NAV1.8 protein; or
f. Sentence 90q, 90r or sentence 90s, wherein the second polypeptide is a human NAV1.9 protein.

Sentence 92. An antibody or fragment according to sentence 90 or sentence 91 which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 93. An antibody or fragment according to sentence 92, wherein the antibody or fragment binds to the D1E2 loop (SEQ ID No:4) of human NAV1.7.

Sentence 94. An antibody or fragment according to any one of sentences 90 to 93, wherein the first polypeptide is a NAV1.7 protein.

Sentence 95. An antibody or fragment according to any one of sentences 90 to 94, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 96. An antibody or fragment thereof according to any one of sentences 90 to 95, which has the features of one or both of sentence 90a and sentence 90b, and one, more or all of the features of sentence 90g, sentence 90h and sentence 90i.

Sentence 97. An antibody or fragment thereof according to any one of sentences 90 to 95, which has the features of one or both of sentence 90a and sentence 90b; and one, more (e.g. 2 or 3) or all of the features of sentence 90g, sentence 90j, sentence 90k and sentence 90l.

Sentence 98. An antibody or fragment thereof according to any one of sentences 90 to 95 which
a. has the features of sentence 84g; or
b. has one or both of the features of sentence 90h and sentence 90i, and one, more (e.g. 2 or 3) or all of the features of sentence 90j, sentence 90k and sentence 90l.

Sentence 99. An antibody or fragment thereof according to any one of sentences 90 to 95, which has the features of one or both of sentence 90a and sentence 90b; and one, more (e.g. 2 or 3) or all of the features of sentence 90g, sentence 90j, sentence 90k and sentence 90l; and one, more (e.g. 2) or all of the features of sentence 90j, sentence 90k and sentence 90l.

Sentence 107. An antibody or fragment thereof which binds to human NAV1.7 (SEQ ID No:2), and comprises one, more (e.g. 2, 3, 4 or 5), or all of the features a. to cc.:
a. is selective for a first polypeptide comprising the sequence VELF (SEQ ID No:176, NAV1.7 motif) over a second polypeptide comprising the sequence MELS (SEQ ID No:177, NAV1.6 motif);
b. is selective for a first polypeptide comprising the sequence VELFLADVEG (SEQ ID No:8, NAV1.7 motif) over a second polypeptide comprising the sequence MELSLADVEG (SEQ ID No:152, NAV1.6 motif);
c. is selective for a first polypeptide comprising the sequence VELF (SEQ ID No:176, NAV1.7 motif) over a second polypeptide comprising the sequence VELG (SEQ ID No:88, NAV1.1 motif);
d. is selective for a first polypeptide comprising the sequence FLAD (SEQ ID No:178, NAV1.7 motif) over a second polypeptide comprising the sequence GLAN (SEQ ID No:179, NAV1.1, NAV1.2 and NAV1.4 motif);
e. is selective for a first polypeptide comprising the sequence FLADV (SEQ ID No:259, NAV1.7 motif) over a second polypeptide comprising the sequence GLANV (SEQ ID No:250, NAV1.1, NAV1.2 and NAV1.4 motif);
f. is selective for a first polypeptide comprising the sequence LADVEG (SEQ ID No:243, NAV1.7 motif) over a second polypeptide comprising the sequence LANVEG (SEQ ID No:248, NAV1.1 and NAV1.2 motif);
g. is selective for a first polypeptide comprising the sequence VELFLADVEG (SEQ ID No:8, NAV1.7 motif) over a second polypeptide comprising the sequence VELGLANVEG (SEQ ID No:62, NAV1.1 motif)
h. is selective for a first polypeptide comprising the sequence VELF (SEQ ID No:176, NAV1.7 motif) over a second polypeptide comprising the sequence MELG (SEQ ID No:253, NAV1.2, NAV1.3 and NAV1.5 motif);
i. is selective for a first polypeptide comprising the sequence FLADVEG (SEQ ID No:180, NAV1.7 motif) over a second polypeptide comprising the sequence MELGLAN (SEQ ID No:181, NAV1.2 motif);
j. is selective for a first polypeptide comprising the sequence VELFLADVEG (SEQ ID No:8, NAV1.7 motif) over a second polypeptide comprising the sequence MELGLANVEG (SEQ ID No:80, NAV1.2 motif);
k. is selective for a first polypeptide comprising the sequence VELFLAD (SEQ ID No:180, NAV1.7 motif) over a second polypeptide comprising the sequence MELGLSN (SEQ ID No:182, NAV1.3 motif);
l. is selective for a first polypeptide comprising the sequence FLADV (SEQ ID No:259, NAV1.7 motif) over a second polypeptide comprising the sequence GLSNV (SEQ ID No:255, NAV1.3 motif);
m. is selective for a first polypeptide comprising the sequence LADVEG (SEQ ID No:243, NAV1.7 motif) over a second polypeptide comprising the sequence LSNVEG (SEQ ID No:254, NAV1.3 motif);
n. is selective for a first polypeptide comprising the sequence VELFLADVEG (SEQ ID No:8, NAV1.7 motif) over a second polypeptide comprising the sequence MELGLSNVEG (SEQ ID No:98, NAV1.3 motif);
o. is selective for a first polypeptide comprising the sequence FLADVE (SEQ ID No:183, NAV1.7 motif) over a second polypeptide comprising the sequence GLANVQ (SEQ ID No:184, NAV1.4 motif);
p. is selective for a first polypeptide comprising the sequence LADVEG (SEQ ID No:243, NAV1.7 motif) over a second polypeptide comprising the sequence LANVQG (SEQ ID No:256, NAV1.4 motif);
q. is selective for a first polypeptide comprising the sequence VELFLADVEG (SEQ ID No:8, NAV1.7 motif) over a second polypeptide comprising the sequence VELGLANVQG (SEQ ID No:116, NAV1.4 motif);
r. is selective for a first polypeptide comprising the sequence LADVEG (SEQ ID No:243, NAV1.7 motif) over a second polypeptide comprising the sequence LSRMSN (SEQ ID No:258, NAV1.5 motif);
s. is selective for a first polypeptide comprising the sequence FLADV (SEQ ID No:259, NAV1.7 motif) over a second polypeptide comprising the sequence GLSRM (SEQ ID No:257, NAV1.5 motif);
t. is selective for a first polypeptide comprising the sequence VELFLAD (SEQ ID No:180, NAV1.7 motif) over a second polypeptide comprising the sequence MELGLSR (SEQ ID No:94, NAV1.5 motif);r
u. is selective for a first polypeptide comprising the sequence VELFLADVEG (SEQ ID No:8, NAV1.7 motif) over a second polypeptide comprising the sequence MELGLSRMSN (SEQ ID No:134, NAV1.5 motif);
v. is selective for a first polypeptide comprising the sequence VELF (SEQ ID No:176, NAV1.7 motif) over a second polypeptide comprising the sequence LELG (SEQ ID No:241, NAV1.8 motif);
w. is selective for a first polypeptide comprising the sequence LADVEG (SEQ ID No:243, NAV1.7 motif) over a second polypeptide comprising the sequence VAKKGS (SEQ ID No:242, NAV1.8 motif);
x. is selective for a first polypeptide comprising the sequence FLADV (SEQ ID No:259, NAV1.7 motif) over a second polypeptide comprising the sequence GVAKK (SEQ ID No:249, NAV1.8 motif);
y. is selective for a first polypeptide comprising the sequence VELFLADVEG (SEQ ID No:8, NAV1.7 motif) over a second polypeptide comprising the sequence LELGVAKKGS (SEQ ID No:26, NAV1.8 motif);
z. is selective for a first polypeptide comprising the sequence VELF (SEQ ID No:176, NAV1.7 motif) over a second polypeptide comprising the sequence ADVM (SEQ ID No:76, NAV1.9 motif);
aa. is selective for a first polypeptide comprising the sequence VELFLAD (SEQ ID No:180, NAV1.7 motif) over a second polypeptide comprising the sequence ADVMNCV (SEQ ID No:260, NAV1.9 motif);
bb. is selective for a first polypeptide comprising the sequence LADVEG (SEQ ID No:243, NAV1.7 motif) over a second polypeptide comprising the sequence VLQKRS (SEQ ID No:261, NAV1.9 motif); and
cc. is selective for a first polypeptide comprising the sequence VELFLADVEG (SEQ ID No:8, NAV1.7 motif) over a second polypeptide comprising the sequence ADVMNCVLQKRS (SEQ ID No:44, NAV1.9 motif).

Sentence 108. An antibody or fragment according to:
a. Sentence 107a or sentence 107b, wherein the second polypeptide is a human NAV1.6 protein; or
b. Sentence 107c, 107d, 107e, 107f or sentence 107g, wherein the second polypeptide is a human NAV1.1 protein; or
c. Sentence 107c, 107d, 107e, 107f, 107h, 107i, or sentence 107j, wherein the second polypeptide is a human NAV1.2 protein; or
d. Sentence 107h, 107k, 107l, 107m or sentence 107n, wherein the second polypeptide is a human NAV1.3 protein; or
e. Sentence 107d, 107e, 107o, 107p or sentence 107q, wherein the second polypeptide is a human NAV1.4 protein; or
f. Sentence 107h, 107r, 107s, 107t or sentence 107u, wherein the second polypeptide is a human NAV1.5 protein; or
g. Sentence 107v, 107w, 107x or sentence 107y, wherein the second polypeptide is a human NAV1.8 protein; or
h. Sentence 107z, 107aa, 107bb or sentence 107cc, wherein the second polypeptide is a human NAV1.9 protein.

Sentence 109. An antibody or fragment according to sentence 107 or sentence 108 which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 110. An antibody or fragment according to sentence 109, wherein the antibody or fragment binds to the D2E2 loop (SEQ ID No:8) of human NAV1.7.

Sentence 111. An antibody or fragment according to any one of sentences 107 to 110, wherein the first polypeptide is a NAV1.7 protein.

Sentence 112. An antibody or fragment according to any one of sentences 107 to 111, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 113. An antibody or fragment thereof according to any one of sentences 107 to 112, which has the features of sentence 107a and/or sentence 107b; and one, more (e.g. 2 or 3) or all of the features of sentence 107d, sentence 107e, sentence 107o, sentence 107p and sentence 107q.

Sentence 114. An antibody or fragment thereof according to any one of sentences 107 to 112, which has the features of sentence 107a and/or sentence 107b; and one, more (e.g. 2 or 3) or all of the features of sentence 107h, sentence 107r, sentence 107s, sentence 107t and sentence 107u.

Sentence 115. An antibody or fragment thereof according to any one of sentences 107 to 112, which has the features of sentence 107d, sentence 107e, sentence 107o, sentence 107p and/or sentence 107q; and one, more (e.g. 2 or 3) or all of the features of sentence 107h, sentence 107r, sentence 107s, sentence 107t and sentence 107u.

Sentence 116. An antibody or fragment thereof according to any one of sentences 107 to 112, which has the features of sentence 107d, sentence 107e, sentence 107o, sentence 107p and/or sentence 107q; and one, more (e.g. 2 or 3) or all of the features of sentence 107h, sentence 107r, sentence 107s, sentence 107t and sentence 107u; and one or both of the features of sentence 107a and sentence 107b.

Sentence 124. An antibody or fragment thereof which binds to human NAV1.7 (SEQ ID No:2 and comprises one, more (e.g. 2, 3, 4 or 5), or all of the features a. to pp.:
a. binds to a first polypeptide comprising the sequence VTLVA (SEQ ID No:193, NAV1.7 motif) over a second polypeptide comprising the sequence VSLIA (SEQ ID No:262, NAV1.6 motif);
b. binds to a first polypeptide comprising the sequence GYSDL (SEQ ID No:198, NAV1.7 motif) over a second polypeptide comprising the sequence GYSEL (SEQ ID No:88, NAV1.6, NAV1.1, NAV1.2, NAV1.3 and NAV1.4 motif);
c. binds to a first polypeptide comprising the sequence TLVANT (SEQ ID No:185, NAV1.7 motif) over a second polypeptide comprising the sequence SLIANA (SEQ ID No:186, NAV1.6 motif);
d. binds to a first polypeptide comprising the sequence TLVAN (SEQ ID No:200, NAV1.7 motif) over a second polypeptide comprising the sequence SLIAN (SEQ ID No:320, NAV1.6 motif);
e. binds to a first polypeptide comprising the sequence SDLGP (SEQ ID No:187, NAV1.7 motif) over a second polypeptide comprising the sequence SELGA (SEQ ID No:188, NAV1.6, NAV1.1, NAV1.2 and NAV1.3 motif);
f. binds to a first polypeptide comprising the sequence TLGYSD (SEQ ID No:191, NAV1.7 motif) over a second polypeptide comprising the sequence ALGYSE (SEQ ID No:192, NAV1.6, NAV1.1, NAV1.2 and NAV1.3 motif);
g. binds to a first polypeptide comprising the sequence TLVANTLGYSDLGP (SEQ ID No:231, NAV1.7 motif) over a second polypeptide comprising the sequence SLIANALGYSELGA (SEQ ID No:327, NAV1.6 motif);
h. binds to a first polypeptide comprising the sequence VTLVANTLGYSDLGPIK (SEQ ID No:12, NAV1.7 motif) over a second polypeptide comprising the sequence VSLIANALGYSELGAIK (SEQ ID No:156, NAV1.6 motif);
i. binds to a first polypeptide comprising the sequence TLVANT (SEQ ID No:185, NAV1.7 motif) over a second polypeptide comprising the sequence SLTANA (SEQ ID No:189, NAV1.1 and NAV1.2 motif);
j. binds to a first polypeptide comprising the sequence VTLVA (SEQ ID No:193, NAV1.7 motif) over a second polypeptide comprising the sequence VSLTA (SEQ ID No:268, NAV1.1 and NAV1.2 motif);
k. binds to a first polypeptide comprising the sequence TLVAN (SEQ ID No:200, NAV1.7 motif) over a second polypeptide comprising the sequence SLTAN (SEQ ID No:323, NAV1.1 and NAV1.2 motif);
l. binds to a first polypeptide comprising the sequence TLVANTLGYSDLGP (SEQ ID No:231, NAV1.7 motif) over a second polypeptide comprising the sequence SLTANALGYSELGA (SEQ ID No:233, NAV1.1 and NAV1.2 motif);
m. binds to a first polypeptide comprising the sequence VTLVANTLGYSDLGPIK (SEQ ID No:12, NAV1.7 motif) over a second polypeptide comprising the sequence VSLTANALGYSELGAIK (SEQ ID No:66, NAV1.1 and NAV1.2 motif);
n. binds to a first polypeptide comprising the sequence TLVANT (SEQ ID No:185, NAV1.7 motif) over a second polypeptide comprising the sequence SLVANA (SEQ ID No:182, NAV1.3 motif);
o. binds to a first polypeptide comprising the sequence VTLVA (SEQ ID No:193, NAV1.7 motif) over a second polypeptide comprising the sequence VSLVA (SEQ ID No:270, NAV1.3 and NAV1.5 motif);
p. binds to a first polypeptide comprising the sequence TLVANTLGYSDLGP (SEQ ID No:231, NAV1.7 motif) over a second polypeptide comprising the sequence SLVANALGYSELGA (SEQ ID No:232, NAV1.3 motif);
q. binds to a first polypeptide comprising the sequence TLVANTLGYSDLGPIK (SEQ ID No:12, NAV1.7 motif) over a second polypeptide comprising the sequence SLVANALGYSELGAIK (SEQ ID No:102, NAV1.3 motif);
r. binds to a first polypeptide comprising the sequence VTLVA (SEQ ID No:193, NAV1.7 motif) over a second polypeptide comprising the sequence ISLVA (SEQ ID No:194, NAV1.4 motif);
s. binds to a first polypeptide comprising the sequence TLVANT (SEQ ID No:185, NAV1.7 motif) over a second polypeptide comprising the sequence SLVANW (SEQ ID No:195, NAV1.4 motif);
t. binds to a first polypeptide comprising the sequence TLGYSD (SEQ ID No:191, NAV1.7 motif) over a second polypeptide comprising the sequence WLGYSE (SEQ ID No:197, NAV1.4 motif);
u. binds to a first polypeptide comprising the sequence SDLGP (SEQ ID No:187, NAV1.7 motif) over a second polypeptide comprising the sequence SELGP (SEQ ID No:273, NAV1.4 motif);
v. binds to a first polypeptide comprising the sequence VTLVANTLGYSD (SEQ ID No:227, NAV1.7 motif) over a second polypeptide comprising the sequence ISLVANWLGYSE (SEQ ID No:228, NAV1.4 motif);
w. binds to a first polypeptide comprising the sequence VTLVANTLGYSDLGPIK (SEQ ID No:12, NAV1.7 motif) over a second polypeptide comprising the sequence ISLVANWLGYSELGPIK (SEQ ID No:120, NAV1.4 motif);
x. binds to a first polypeptide comprising the sequence TLVANT (SEQ ID No:185, NAV1.7 motif) over a second polypeptide comprising the sequence SLVANT (SEQ ID No:277, NAV1.5 motif);
y. binds to a first polypeptide comprising the sequence SDLGP (SEQ ID No:187, NAV1.7 motif) over a second polypeptide comprising the sequence AEMGP (SEQ ID No:276, NAV1.5 motif);
z. binds to a first polypeptide comprising the sequence GYSDL (SEQ ID No:198, NAV1.7 motif) over a second polypeptide comprising the sequence GFAEM (SEQ ID No:199, NAV1.5 motif);
aa. binds to a first polypeptide comprising the sequence TLVAN (SEQ ID No:200, NAV1.7 motif) over a second polypeptide comprising the sequence SLVAN (SEQ ID No:201, NAV1.3, NAV1.4 and NAV1.5 motif);
bb. binds to a first polypeptide comprising the sequence TLGYSD (SEQ ID No:191, NAV1.7 motif) over a second polypeptide comprising the sequence TLGFAE (SEQ ID No:84, NAV1.5 motif);
cc. binds to a first polypeptide comprising the sequence TLVANTLGYSDL (SEQ ID No:325, NAV1.7 motif) over a second polypeptide comprising the sequence SLVANTLGFAEM (SEQ ID No:326, NAV1.5 motif);
dd. binds to a first polypeptide comprising the sequence VTLVANTLGYSDLGPIK (SEQ ID No:12, NAV1.7 motif) over a second polypeptide comprising the sequence VSLVANTLGFAEMGPIK (SEQ ID No:138, NAV1.5 motif);
ee. binds to a first polypeptide comprising the sequence VTLVA (SEQ ID No:193, NAV1.7 motif) over a second polypeptide comprising the sequence ISLTA (SEQ ID No:202, NAV1.8 motif);
ff. binds to a first polypeptide comprising the sequence VANTLG (SEQ ID No:203, NAV1.7 motif) over a second polypeptide comprising the sequence TAKILE (SEQ ID No:204, NAV1.8 motif);
gg. binds to a first polypeptide comprising the sequence GYSDLG (SEQ ID No:205, NAV1.7 motif) over a second polypeptide comprising the sequence EYSEVA (SEQ ID No:206, NAV1.8 motif);
hh. binds to a first polypeptide comprising the sequence TLVAN (SEQ ID No:200, NAV1.7 motif) over a second polypeptide comprising the sequence SLTAK (SEQ ID No:324, NAV1.8 motif);
ii. binds to a first polypeptide comprising the sequence TLVANT (SEQ ID No:185, NAV1.7 motif) over a second polypeptide comprising the sequence SLTAKI (SEQ ID No:265, NAV1.8 motif);
jj. binds to a first polypeptide comprising the sequence SDLGP (SEQ ID No:187, NAV1.7 motif) over a second polypeptide comprising the sequence SEVAP (SEQ ID No:264, NAV1.8 motif);
kk. binds to a first polypeptide comprising the sequence TLGYSD (SEQ ID No:191, NAV1.7 motif) over a second polypeptide comprising the sequence ILEYSE (SEQ ID No:328, NAV1.8 motif);
ii. binds to a first polypeptide comprising the sequence VTLVANTLGYSDLG (SEQ ID No:158, NAV1.7 motif) over a second polypeptide comprising the sequence ISLTAKILEYSEVA (SEQ ID No:162, NAV1.8 motif);
mm. binds to a first polypeptide comprising the sequence VTLVANTLGYSDLGPIK (SEQ ID No:12, NAV1.7 motif) over a second polypeptide comprising the sequence ISLTAKILEYSEVAPIK (SEQ ID No:30, NAV1.8 motif);
nn. binds to a first polypeptide comprising the sequence VTLVA (SEQ ID No:193, NAV1.7 motif) over a second polypeptide comprising the sequence TTLIN (SEQ ID No:207, NAV1.9 motif);
oo. binds to a first polypeptide comprising the sequence LGPI (SEQ ID No:208, NAV1.7 motif) over a second polypeptide comprising the sequence LMEL (SEQ ID No:209, NAV1.9 motif); and
pp. binds to a first polypeptide comprising the sequence VTLVANTLGYSDLGPIK (SEQ ID No:12, NAV1.7 motif) over a second polypeptide comprising the sequence TTLINLMELK (SEQ ID No:48, NAV1.9 motif).

Sentence 125. An antibody or fragment according to:
a. Sentence 124a, 124b, 124c, 124d, 124e, 124f, 124g or sentence 124h, wherein the second polypeptide is a human NAV1.6 protein; or
b. Sentence 124b, 124e, 124f, 124i, 124j, 124k, 124l or sentence 124m, wherein the second polypeptide is a human NAV1.1 protein or a human NAV1.2 protein; or
c. Sentence 124b, 124e, 124f, 124n, 124o, 124p, 124q or sentence 124aa, wherein the second polypeptide is a human NAV1.3 protein; or
d. Sentence 124b, 124r, 124s, 124t, 124u, 124v, 124w or sentence 124aa, wherein the second polypeptide is a human NAV1.4 protein; or
e. Sentence 124o, 124x, 124y, 124z, 124aa, 124bb, 124cc or sentence 124dd, wherein the second polypeptide is a human NAV1.5 protein; or
f. Sentence 124ee, 124ff, 124gg, 124hh, 124ii, 124jj, 124kk, 124ll or sentence 124 mm, wherein the second polypeptide is a human NAV1.8 protein; or
g. Sentence 124nn, 124oo or sentence 124pp, wherein the second polypeptide is a human NAV1.9 protein.

Sentence 126. An antibody or fragment according to sentence 124 or sentence 125 which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 127. An antibody or fragment according to sentence 126, wherein the antibody or fragment binds to the D3E2 loop (SEQ ID No:12) of human NAV1.7.

Sentence 128. An antibody or fragment according to any one of sentences 124 to 127, wherein the first polypeptide is a NAV1.7 protein.

Sentence 129. An antibody or fragment according to any one of sentences 124 to 128, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 130. An antibody or fragment thereof according to any one of sentences 124 to 129 which
a. has the features of sentence 124b; or
b. has the features of sentence 124a, sentence 124c, sentence 124d, sentence 124e, sentence 124f, sentence 124g and/or sentence 124h, and one, more (e.g. 2, 3, or 4) or all of the features of sentence 124r, sentence 124s, sentence 124t, sentence 124u, sentence 124v and sentence 124w.

Sentence 131. An antibody or fragment thereof according to any one of sentences 124 to 129, which has the features of sentence 124a, sentence 124c, sentence 124d, sentence 124e, sentence 124f, sentence 124g and/or sentence 124h, and one, more (e.g. 2, 3, or 4) or all of the features of sentence 124o, sentence 124x, sentence 124y, sentence 124z, sentence 124bb, sentence 124cc and sentence 124dd.

Sentence 132. An antibody or fragment thereof according to any one of sentences 124 to 129, which:
a. has the features of sentence 124aa; or
b. has the features of sentence 124r, sentence 124s, sentence 124t, sentence 124u, sentence 124v and/or sentence 124w, and one, more (e.g. 2, 3, or 4) or all of the features of sentence 124o, sentence 124x, sentence 124y, sentence 124z, sentence 124bb, sentence 124cc and sentence 124dd.

Sentence 133. An antibody or fragment thereof according to any one of sentences 124 to 129, which:
a. has the features of sentence 124b and of sentence 124aa; or
b. has the features of sentence 124r, sentence 124s, sentence 124t, sentence 124u, sentence 124v and/or sentence 124w; and one, more (e.g. 2, 3, or 4) or all of the features of sentence 124o, sentence 124x, sentence 124y, sentence 124z, sentence 124bb, sentence 124cc and sentence 124dd; and one, more (e.g. 2, 3, or 4) or all of the features of sentence 124a, sentence 124c, sentence 124d, sentence 124e, sentence 124f, sentence 124g and sentence 124h.

Sentence 141. An antibody or fragment thereof which binds to human NAV1.7 (SEQ ID No:2), and comprises one, more (e.g. 2, 3, 4 or 5), or all of the features a. to ee.:
a. is selective for a first polypeptide comprising the sequence LIET (SEQ ID No:210, NAV1.7 motif) over a second polypeptide comprising the sequence IIEK (SEQ ID No:211, NAV1.6 motif);
b. is selective for a first polypeptide comprising the sequence VGMFLADLIETYFVSPTLFR (SEQ ID No:16, NAV1.7 motif) over a second polypeptide comprising the sequence VGMFLADIIEKYFVSPTLFR (SEQ ID No:160, NAV1.6 motif);
c. is selective for a first polypeptide comprising the sequence DLIET (SEQ ID No:212, NAV1.7 motif) over a second polypeptide comprising the sequence ELIEK (SEQ ID No:213, NAV1.1 and NAV1.2 motif);
d. is selective for a first polypeptide comprising the sequence MFLADLIET (SEQ ID No:229, NAV1.7 motif) over a second polypeptide comprising the sequence MFLAELIEK (SEQ ID No:154, NAV1.1 and NAV1.2 motif);
e. is selective for a first polypeptide comprising the sequence ADLIET (SEQ ID No:221), NAV1.7 motif) over a second polypeptide comprising the sequence AELIEK (SEQ ID No:150, NAV1.1 and NAV1.2 motif);
f. is selective for a first polypeptide comprising the sequence MFLADLIETYFV (SEQ ID No:225, NAV1.7 motif) over a second polypeptide comprising the sequence MFLAELIEKYFV (SEQ ID No:144, NAV1.1 and NAV1.2 motif);
g. is selective for a first polypeptide comprising the sequence VGMFLADLIETYFVSPTLFR (SEQ ID No:16, NAV1.7 motif) over a second polypeptide comprising the sequence VGMFLAELIEKYFVSPTLFR (SEQ ID No:160, NAV1.1 and NAV1.2 motif);
h. is selective for a first polypeptide comprising the sequence DLIET (SEQ ID No:212, NAV1.7 motif) over a second polypeptide comprising the sequence EMIEK (SEQ ID No:214, NAV1.3 motif);
i. is selective for a first polypeptide comprising the sequence MFLADLIET (SEQ ID No:229, NAV1.7 motif) over a second polypeptide comprising the sequence MFLAEMIEK (SEQ ID No:140, NAV1.3 motif);
j. is selective for a first polypeptide comprising the sequence ADLIET (SEQ ID No:221, NAV1.7 motif) over a second polypeptide comprising the sequence AEMIEK (SEQ ID No:136, NAV1.3 motif);
k. is selective for a first polypeptide comprising the sequence MFLADLIETYFV (SEQ ID No:225, NAV1.7 motif) over a second polypeptide comprising the sequence MFLAEMIEKYFV (SEQ ID No:132, NAV1.3 motif);
l. is selective for a first polypeptide comprising the sequence VGMFLADLIETYFVSPTLFR (SEQ ID No:16, NAV1.7 motif) over a second polypeptide comprising the sequence VGMFLAEMIEKYFVSPTLFR (SEQ ID No:106, NAV1.3 motif);
m. is selective for a first polypeptide comprising the sequence MFLAD (SEQ ID No:215, NAV1.7 motif) over a second polypeptide comprising the sequence LALSD (SEQ ID No:216, NAV1.4 motif);
n. is selective for a first polypeptide comprising the sequence LIET (SEQ ID No:210, NAV1.7 motif) over a second polypeptide comprising the sequence LIQK (SEQ ID No:217, NAV1.4 motif);
o. is selective for a first polypeptide comprising the sequence ADLIE (SEQ ID No:218, NAV1.7 motif) over a second polypeptide comprising the sequence SDLIQ (SEQ ID No:219, NAV1.4 motif);
p. is selective for a first polypeptide comprising the sequence MFLADLIET (SEQ ID No:229, NAV1.7 motif) over a second polypeptide comprising the sequence LALSDLIQK (SEQ ID No:230, NAV1.4 motif);
q. is selective for a first polypeptide comprising the sequence ADLIET (SEQ ID No:221, NAV1.7 motif) over a second polypeptide comprising the sequence SDLIQK (SEQ ID No:126, NAV1.4 motif);
r. is selective for a first polypeptide comprising the sequence ETYFV (SEQ ID No:223, NAV1.7 motif) over a second polypeptide comprising the sequence QKYFV (SEQ ID No:122, NAV1.4 motif);
s. is selective for a first polypeptide comprising the sequence VGMFLADLIETYFVSPTLFR (SEQ ID No:16, NAV1.7 motif) over a second polypeptide comprising the sequence VGLALSDLIQKYFVSPTLFR (SEQ ID No:124, NAV1.4 motif);
t. is selective for a first polypeptide comprising the sequence MFLAD (SEQ ID No:215, NAV1.7 motif) over a second polypeptide comprising the sequence TVLSD (SEQ ID No:220, NAV1.5 motif);
u. is selective for a first polypeptide comprising the sequence ADLIET (SEQ ID No:221, NAV1.7 motif) over a second polypeptide comprising the sequence SDIIQK (SEQ ID No:222, NAV1.5 motif);
v. is selective for a first polypeptide comprising the sequence ETYFV (SEQ ID No:223, NAV1.7 motif) over a second polypeptide comprising the sequence QKYFF (SEQ ID No:224, NAV1.5 motif);
w. is selective for a first polypeptide comprising the sequence MFLADLIETYFV (SEQ ID No:225, NAV1.7 motif) over a second polypeptide comprising the sequence TVLSDIIQKYFF (SEQ ID No:226, NAV1.5 motif);
x. is selective for a first polypeptide comprising the sequence MFLADLIET (SEQ ID No:229, NAV1.7 motif) over a second polypeptide comprising the sequence TVLSDIIQK (SEQ ID No:118, NAV1.5 motif);
y. is selective for a first polypeptide comprising the sequence VGMFLADLIETYFVSPTLFR (SEQ ID No:16, NAV1.7 motif) over a second polypeptide comprising the sequence VGTVLSDIIQKYFFSPTLFR (SEQ ID No:142, NAV1.5 motif);
z. is selective for a first polypeptide comprising the sequence VGMFLADLIETYFV (SEQ ID No:114, NAV1.7 motif) over a second polypeptide comprising the sequence ASLIFSAILKSLQSYF (SEQ ID No:108, NAV1.8 motif);
aa. is selective for a first polypeptide comprising the sequence MFLADLIET (SEQ ID No:229, NAV1.7 motif) over a second polypeptide comprising the sequence LIFSAILKS (SEQ ID No:104, NAV1.8 motif);
bb. is selective for a first polypeptide comprising the sequence VGMFLADLIETYFVSPTLFR (SEQ ID No:16, NAV1.7 motif) over a second polypeptide comprising the sequence ASLIFSAILKSLQSYFSPTLFR (SEQ ID No:34, NAV1.8 motif);
cc. is selective for a first polypeptide comprising the sequence VGMFLADLIETYFVS (SEQ ID No:100, NAV1.7 motif) over a second polypeptide comprising the sequence VSTMISTLENQEHIPFP (SEQ ID No:96, NAV1.8 motif);
dd. is selective for a first polypeptide comprising the sequence MFLADLIET (SEQ ID No:229, NAV1.7 motif) over a second polypeptide comprising the sequence TMISTLEN (SEQ ID No:90, NAV1.8 motif); and
ee. is selective for a first polypeptide comprising the sequence VGMFLADLIETYFVSPTLFR (SEQ ID No:16, NAV1.7 motif) over a second polypeptide comprising the sequence STMISTLENQEHIPFPPTLFR (SEQ ID No:52, NAV1.8 motif).

Sentence 142. An antibody or fragment according to:
a. Sentence 141a or sentence 141b, wherein the second polypeptide is a human NAV1.6 protein; or
b. Sentence 141c, 141d, 141e, 141f or sentence 141g, wherein the second polypeptide is a human NAV1.1 protein or a NAV1.2 protein; or
c. Sentence 141h, 141i, 141j, 141k or sentence 141l, wherein the second polypeptide is a human NAV1.3 protein; or
d. Sentence 141m, 141n, 141o, 141p, 141q, 141r or sentence 141s, wherein the second polypeptide is a human NAV1.4 protein; or
e. Sentence 141t, 141u, 141v, 141w, 141x or sentence 141y, wherein the second polypeptide is a human NAV1.5 protein; or
f. Sentence 141z, 141aa or sentence 141bb, wherein the second polypeptide is a human NAV1.8 protein; or
g. Sentence 141cc, 141dd or sentence 141ee, wherein the second polypeptide is a human NAV1.9 protein.

Sentence 143. An antibody or fragment according to sentence 141 or sentence 142 which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 144. An antibody or fragment according to sentence 143, wherein the antibody or fragment binds to the D4E2 loop (SEQ ID No:16) of human NAV1.7.

Sentence 145. An antibody or fragment according to any one of sentences 141 to 144, wherein the first polypeptide is a NAV1.7 protein.

Sentence 146. An antibody or fragment according to any one of sentences 141 to 145, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 147. An antibody or fragment thereof according to any one of sentences 141 to 146, which has the features of sentence 141a and/or sentence 141b; and one, more (e.g. 2, 3 or 4) or all of the features of sentence 141m, sentence 141n, sentence 141o, sentence 141p, sentence 141q, sentence 141r and sentence 141s.

Sentence 148. An antibody or fragment thereof according to any one of sentences 141 to 146, which has the features of sentence 141a and/or sentence 141b; and one, more (e.g. 2, 3 or 4) or all of the features of sentence 141t, sentence 141u, sentence 141v, sentence 141w, sentence 141x and sentence 141y.

Sentence 149. An antibody or fragment thereof according to any one of sentences 141 to 146, which has the features of one, more (e.g. 2, 3 or 4) or all of sentence 141m, sentence 141n, sentence 141o, sentence 141p, sentence 141q, sentence 141r and sentence 141s; and one, more (e.g. 2, 3 or 4) or all of the features of sentence 141t, sentence 141u, sentence 141v, sentence 141w, sentence 141x and sentence 141y.

Sentence 150. An antibody or fragment thereof according to any one of sentences 141 to 146, which has the features of one, more (e.g. 2, 3 or 4) or all of sentence 141m, sentence 141n, sentence 141o, sentence 141p, sentence 141q, sentence 141r and sentence 141s; and one, more (e.g. 2, 3 or 4) or all of the features of sentence 141t, sentence 141u, sentence 141v, sentence 141w, sentence 141x and sentence 141y; and the features of one or both of sentence 141a and sentence 141b.

Sentence 158. An antibody or fragment thereof which binds to human NAV1.8 (SEQ ID No:20), and comprises one, more (e.g. 2, 3, 4 or 5), or all of the features a. to o.:
a. is selective for a first polypeptide comprising the sequence VGTAIDLRG (SEQ ID No:22, NAV1.8 motif) over a second polypeptide comprising the sequence ITEFVNLGN (SEQ ID No:148, NAV1.6 motif);
b. is selective for a first polypeptide comprising the sequence VGTAID (SEQ ID No:234, NAV1.8 motif) over a second polypeptide comprising the sequence ITEFVN (SEQ ID No:235, NAV1.6 motif);
c. is selective for a first polypeptide comprising the sequence IDLRG (SEQ ID No:236, NAV1.8 motif) over a second polypeptide comprising the sequence VNLGN (SEQ ID No:172, NAV1.6 and NAV1.7 motif);
d. is selective for a first polypeptide comprising the sequence VGTAIDLRG (SEQ ID No:22, NAV1.8 motif) over a second polypeptide comprising the sequence VTEFVDLGN (SEQ ID No:58, NAV1.1, NAV1.2 and NAV1.3 motif);
e. is selective for a first polypeptide comprising the sequence VGTAID (SEQ ID No:234, NAV1.8 motif) over a second polypeptide comprising the sequence VTEFVD (SEQ ID No:172, NAV1.1, NAV1.2 and NAV1.3 motif);
f. is selective for a first polypeptide comprising the sequence IDLRG (SEQ ID No:236, NAV1.8 motif) over a second polypeptide comprising the sequence VDLGN (SEQ ID No:238, NAV1.1, NAV1.2, NAV1.3, NAV1.4 and NAV1.5 motif);
g. is selective for a first polypeptide comprising the sequence VGTAIDLRG (SEQ ID No:22, NAV1.8 motif) over a second polypeptide comprising the sequence VTEFVDLGN (SEQ ID No:112, NAV1.4 motif);
h. is selective for a first polypeptide comprising the sequence VGTAID (SEQ ID No:234, NAV1.8 motif) over a second polypeptide comprising the sequence LTEFVD (SEQ ID No:239, NAV1.4 motif);
i. is selective for a first polypeptide comprising the sequence VGTAIDLRG (SEQ ID No:22, NAV1.8 motif) over a second polypeptide comprising the sequence TTEFVDLGN (SEQ ID No:130, NAV1.5 motif);
j. is selective for a first polypeptide comprising the sequence VGTAID (SEQ ID No:234, NAV1.8 motif) over a second polypeptide comprising the sequence TTEFVD (SEQ ID No:175, NAV1.5 motif);
k. is selective for a first polypeptide comprising the sequence VGTAIDLRG (SEQ ID No:22, NAV1.8 motif) over a second polypeptide comprising the sequence LTEFVNLGN (SEQ ID No:4, NAV1.7 motif);
l. is selective for a first polypeptide comprising the sequence VGTAID (SEQ ID No:234, NAV1.8 motif) over a second polypeptide comprising the sequence LTEFVN (SEQ ID No:171, NAV1.7 motif);
m. is selective for a first polypeptide comprising the sequence VGTAID (SEQ ID No:234, NAV1.8 motif) over a second polypeptide comprising the sequence VSYIPG (SEQ ID No:86, NAV1.9 motif);
n. is selective for a first polypeptide comprising the sequence IDLRG (SEQ ID No:236, NAV1.8 motif) over a second polypeptide comprising the sequence GITIK (SEQ ID No:302, NAV1.9 motif); and
o. is selective for a first polypeptide comprising the sequence VGTAIDLRG (SEQ ID No:22, NAV1.8 motif) over a second polypeptide comprising the sequence VSYIPGMK (SEQ ID No:40, NAV1.9 motif).

Sentence 159. An antibody or fragment according to:
h. Sentence 158a, 158b or sentence 158c, wherein the second polypeptide is a human NAV1.6 protein; or
i. Sentence 158d, 158e or sentence 158f, wherein the second polypeptide is a human NAV1.1 protein, a human NAV1.2 protein or a human NAV1.3 protein; or
j. Sentence 158f, 158g or sentence 158h, wherein the second polypeptide is a human NAV1.4 protein; or
k. Sentence 158f, 158i or sentence 158j, wherein the second polypeptide is a human NAV1.5 protein; or
l. Sentence 158c, 158k or sentence 158l, wherein the second polypeptide is a human NAV1.7 protein; or
m. Sentence 159m, 158n or sentence 158o, wherein the second polypeptide is a human NAV1.9 protein.

Sentence 160. An antibody or fragment according to sentence 158 or sentence 159 which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 161. An antibody or fragment according to sentence 160, wherein the antibody or fragment binds to the D1E2 loop (SEQ ID No:22) of human NAV1.8.

Sentence 162. An antibody or fragment according to any one of sentences 158 to 161, wherein the first polypeptide is a NAV1.8 protein.

Sentence 163. An antibody or fragment according to any one of sentences 158 to 162, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 164. An antibody or fragment thereof according to any one of sentences 158 to 163, which has the one, two or all of the features of sentence 158a, sentence 158b and sentence 158c, and one or both of the features of sentence 158g and sentence 158h.

Sentence 165. An antibody or fragment thereof according to any one of sentences 158 to 163, which has one, two or all of the features of sentence 158a, sentence 158b and sentence 158c, and one or both of the features of sentence 158i and sentence 158j.

Sentence 166. An antibody or fragment thereof according to any one of sentences 158 to 163, which
a. has the features of sentence 158f; or
b. has the features of sentence 158g and/or sentence 158h; and one or both of the features of sentence 158i and sentence 158j.

Sentence 167. An antibody or fragment thereof according to any one of sentences 158 to 163, which has the features of sentence 158g and/or sentence 158h; and one or both of the features of sentence 158i and sentence 158j; and one, two or all of the features of sentence 158a, sentence 158b and sentence 158c.

Sentence 175. An antibody or fragment thereof which binds to human NAV1.8 (SEQ ID No:20), and comprises one, more (e.g. 2, 3, 4 or 5), or all of the features a. to z.:
a. is selective for a first polypeptide comprising the sequence LELG (SEQ ID No:241, NAV1.8 motif) over a second polypeptide comprising the sequence MELS (SEQ ID No:177, NAV1.6 motif);
b. is selective for a first polypeptide comprising the sequence LELGV (SEQ ID No:246, NAV1.8 motif) over a second polypeptide comprising the sequence MELSL (SEQ ID No:251, NAV1.6 motif);
c. is selective for a first r polypeptide comprising the sequence VAKKGS (SEQ ID No:242, NAV1.8 motif) over a second polypeptide comprising the sequence LADVEG (SEQ ID No:243, NAV1.6 and NAV1.7 motif);
d. is selective for a first polypeptide comprising the sequence LGVAK (SEQ ID No:244, NAV1.8 motif) over a second polypeptide comprising the sequence LSLAD (SEQ ID No:245, NAV1.6 motif);
e. is selective for a first polypeptide comprising the sequence LELGVAKKGS (SEQ ID No:26, NAV1.8 motif) over a second polypeptide comprising the sequence MELSLADVEG (SEQ ID No:152, NAV1.6 motif);
f. is selective for a first polypeptide comprising the sequence LELGV (SEQ ID No:246, NAV1.8 motif) over a second polypeptide comprising the sequence VELGL (SEQ ID No:247, NAV1.1 and NAV1.4 motif);
g. is selective for a first polypeptide comprising the sequence VAKKGS (SEQ ID No:242, NAV1.8 motif) over a second polypeptide comprising the sequence LANVEG (SEQ ID No:248, NAV1.1 and NAV1.2 motif);
h. is selective for a first polypeptide comprising the sequence GVAKK (SEQ ID No:249, NAV1.8 motif) over a second polypeptide comprising the sequence GLANV (SEQ ID No:250, NAV1.1, NAV1.2 and NAV1.4 motif);
i. is selective for a first polypeptide comprising the sequence LELGVAKKGS (SEQ ID No:26, NAV1.8 motif) over a second polypeptide comprising the sequence VELGLANVEG (SEQ ID No:62, NAV1.1 motif);
j. is selective for a first polypeptide comprising the sequence LELGV (SEQ ID No:246, NAV1.8 motif) over a second r polypeptide comprising the sequence MELGL (SEQ ID No:252, NAV1.2, NAV1.3 and NAV1.5 motif);
k. is selective for a first polypeptide comprising the sequence LELG (SEQ ID No:241, NAV1.8 motif) over a second polypeptide comprising the sequence MELG (SEQ ID No:253, NAV1.2, NAV1.3 and NAV1.5 motif);
l. is selective for a first polypeptide comprising the sequence LELGVAKKGS (SEQ ID No:26, NAV1.8 motif) over a second polypeptide comprising the sequence MELGLANVEG (SEQ ID No:80, NAV1.2 motif);
m. is selective for a first polypeptide comprising the sequence VAKKGS (SEQ ID No:242, NAV1.8 motif) over a second polypeptide comprising the sequence LSNVEG (SEQ ID No:254, NAV1.3 motif);
n. is selective for a first polypeptide comprising the sequence GVAKK (SEQ ID No:249, NAV1.8 motif) over a second polypeptide comprising the sequence GLSNV (SEQ ID No:255, NAV1.3 motif);
o. is selective for a first polypeptide comprising the sequence LELGVAKKGS (SEQ ID No:26, NAV1.8 motif) over a second polypeptide comprising the sequence MELGLSNVEG (SEQ ID No:98, NAV1.3 motif);
p. is selective for a first polypeptide comprising the sequence VAKKGS (SEQ ID No:242, NAV1.8 motif) over a second polypeptide comprising the sequence LANVQG (SEQ ID No:256, NAV1.4 motif);
q. is selective for a first polypeptide comprising the sequence LELGVAKKGS (SEQ ID No:26, NAV1.8 motif) over a second polypeptide comprising the sequence VELGLANVQG (SEQ ID No:116, NAV1.4 motif);
r. is selective for a first polypeptide comprising the sequence GVAKK (SEQ ID No:249, NAV1.8 motif) over a second polypeptide comprising the sequence GLSRM (SEQ ID No:257, NAV1.5 motif);
s. is selective for a first polypeptide comprising the sequence VAKKGS (SEQ ID No:242, NAV1.8 motif) over a second polypeptide comprising the sequence LSRMSN (SEQ ID No:258, NAV1.5 motif);
t. is selective for a first polypeptide comprising the sequence LELGVAKKGS (SEQ ID No:26, NAV1.8 motif) over a second polypeptide comprising the sequence MELGLSRMSN (SEQ ID No:134, NAV1.5 motif);
u. is selective for a first polypeptide comprising the sequence LELG (SEQ ID No:241, NAV1.8 motif) over a second polypeptide comprising the sequence VELF (SEQ ID No:176, NAV1.7 motif);
v. is selective for a first polypeptide comprising the sequence GVAKK (SEQ ID No:249, NAV1.8 motif) over a second polypeptide comprising the sequence FLADV (SEQ ID No:259, NAV1.7 motif);
w. is selective for a first polypeptide comprising the sequence LELGVAKKGS (SEQ ID No:26, NAV1.8 motif) over a second polypeptide comprising the sequence VELFLADVEG (SEQ ID No:8, NAV1.7 motif);
x. is selective for a first polypeptide comprising the sequence LELGV (SEQ ID No:246, NAV1.8 motif) over a second polypeptide comprising the sequence ADVMNCV (SEQ ID No:260, NAV1.9 motif);
y. is selective for a first polypeptide comprising the sequence VAKKGS (SEQ ID No:242, NAV1.8 motif) over a second polypeptide comprising the sequence VLQKRS (SEQ ID No:261, NAV1.9 motif);
z. is selective for a first polypeptide comprising the sequence LELGVAKKGS (SEQ ID No:26, NAV1.8 motif) over a second polypeptide comprising the sequence ADVMNCVLQKRS (SEQ ID No:44, NAV1.9 motif).

Sentence 176. An antibody or fragment according to:
a. Sentence 175a, 175b, 175c, 175d or sentence 175e, wherein the second polypeptide is a human NAV1.6 protein; or
b. Sentence 175f, 175g, 175h or sentence 175i, wherein the second polypeptide is a human NAV1.1 protein; or
c. Sentence 175g, 175h, 175j, 175k or sentence 175l, wherein the second polypeptide is a human NAV1.2 protein; or
d. Sentence 175j, 175k, 175m, 175n or sentence 175o, wherein the second polypeptide is a human NAV1.3 protein; or
e. Sentence 175f, 175h, 175p or sentence 175q, wherein the second polypeptide is a human NAV1.4 protein; or
f. Sentence 175j, 175k, 175r, 175s or sentence 175t, wherein the second polypeptide is a human NAV1.5 protein; or
g. Sentence 175c, 175u, 175v or sentence 175w, wherein the second polypeptide is a human NAV1.7 protein; or
h. Sentence 175x, 175y or sentence 175z, wherein the second polypeptide is a human NAV1.9 protein.

Sentence 177. An antibody or fragment according to sentence 175 or sentence 176 which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 178. An antibody or fragment according to sentence 177, wherein the antibody or fragment binds to the D2E2 loop (SEQ ID No:26) of human NAV1.8.

Sentence 179. An antibody or fragment according to any one of sentences 175 to 178, wherein the first polypeptide is a NAV1.8 protein.

Sentence 180. An antibody or fragment according to any one of sentences 175 to 179, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 181. An antibody or fragment thereof according to any one of sentences 175 to 180, which has one, more (e.g. 2, 3 or 4) or all of the features of sentence 175a, sentence 175b, sentence 175c, sentence 175d and sentence 175e; and one, more (e.g. 2, 3 or 4) or all of the features of sentence 175f, sentence 175h, sentence 175p and sentence 175q.

Sentence 182. An antibody or fragment thereof according to any one of sentences 175 to 180, which has one, more (e.g. 2, 3 or 4) or all of the features of sentence 175a, sentence 175b, sentence 175c, sentence 175d and sentence 114e; and one, more (e.g. 2, 3 or 4) or all of the features of sentence 175j, sentence 175k, sentence 175r, sentence 175s and sentence 175t.

Sentence 183. An antibody or fragment thereof according to any one of sentences 175 to 180, which has one, more (e.g. 2 or 3) or all of the features of sentence 175f, sentence 175h, sentence 175p and sentence 175q; and one, more (e.g. 2, 3 or 4) or all of the features of sentence 175j, sentence 175k, sentence 175r, sentence 175s and sentence 175t.

Sentence 184. An antibody or fragment thereof according to any one of sentences 175 to 180, which has one, more (e.g. 2 or 3) or all of the features of sentence 175f, sentence 175h, sentence 175p and sentence 175q; and one, more (e.g. 2, 3 or 4) or all of the features of sentence 175j, sentence 175k, sentence 175r, sentence 175s and sentence 175t; and one, more (e.g. 2, 3 or 4) or all of the features of sentence 175a, sentence 175b, sentence 175c, sentence 175d and sentence 175e.

Sentence 192. An antibody or fragment thereof which binds to human NAV1.8 (SEQ ID No:20), and comprises one, more (e.g. 2, 3, 4 or 5), or all of the features a. to gg.:
a. is selective for a first polypeptide comprising the sequence ISLTA (SEQ ID No:202, NAV1.8 motif) over a second polypeptide comprising the sequence VSLIA (SEQ ID No:262, NAV1.6 motif);
b. is selective for a first polypeptide comprising the sequence TAKILE (SEQ ID No:204, NAV1.8 motif) over a second polypeptide comprising the sequence IANALG (SEQ ID No:263, NAV1.6 motif);
c. is selective for a first polypeptide comprising the sequence SEVAP (SEQ ID No:264, NAV1.8 motif) over a second polypeptide comprising the sequence SELGA (SEQ ID No:188, NAV1.6, NAV1.1, NAV1.2 and NAV1.3 motif);
d. is selective for a first polypeptide comprising the sequence SLTAKI (SEQ ID No:265, NAV1.8 motif) over a second polypeptide comprising the sequence SLIANA (SEQ ID No:186, NAV1.6 motif);
e. is selective for a first polypeptide comprising the sequence KILEY (SEQ ID No:266, NAV1.8 motif) over a second polypeptide comprising the sequence NALGY (SEQ ID No:267, NAV1.6, NAV1.1, NAV1.2 and NAV1.3 motif);
f. is selective for a first polypeptide comprising the sequence ISLTAKILEYSEVAPIK (SEQ ID No:30, NAV1.8 motif) over a second polypeptide comprising the sequence VSLIANALGYSELGAIK (SEQ ID No:156, NAV1.6 motif);
g. is selective for a first polypeptide comprising the sequence ISLTA (SEQ ID No:202, NAV1.8 motif) over a second polypeptide comprising the sequence VSLTA (SEQ ID No:268, NAV1.1 and NAV1.2 motif);
h. is selective for a first polypeptide comprising the sequence TAKILE (SEQ ID No:204, NAV1.8 motif) over a second polypeptide comprising the sequence TANALG (SEQ ID No:269, NAV1.1 and NAV1.2 motif);
i. is selective for a first polypeptide comprising the sequence SLTAKI (SEQ ID No:265, NAV1.8 motif) over a second polypeptide comprising the sequence SLTANA (SEQ ID No:189, NAV1.1 and NAV1.2 motif);
j. is selective for a first polypeptide comprising the sequence ISLTAKILEYSEVAPIK (SEQ ID No:30, NAV1.8 motif) over a second polypeptide comprising the sequence VSLTANALGYSELGAIK (SEQ ID No:66, NAV1.1 and NAV1.2 motif);
k. is selective for a first polypeptide comprising the sequence ISLTA (SEQ ID No:202, NAV1.8 motif) over a second polypeptide comprising the sequence VSLVA (SEQ ID No:270, NAV1.3 and NAV1.5 motif);
l. is selective for a first polypeptide comprising the sequence TAKILE (SEQ ID No:204, NAV1.8 motif) over a second polypeptide comprising the sequence VANALG (SEQ ID No:271, NAV1.3 motif);
m. is selective for a first r polypeptide comprising the sequence SLTAKI (SEQ ID No:265, NAV1.8 motif) over a second polypeptide comprising the sequence SLVANA (SEQ ID No:190, NAV1.3 motif);
n. is selective for a first polypeptide comprising the sequence ISLTAKILEYSEVAPIK (SEQ ID No:30, NAV1.8 motif) over a second polypeptide comprising the sequence VSLVANALGYSELGAIK (SEQ ID No:102, NAV1.3 motif);
o. is selective for a first polypeptide comprising the sequence ISLTA (SEQ ID No:202, NAV1.8 motif) over a second polypeptide comprising the sequence ISLVA (SEQ ID No:194, NAV1.4 motif);
p. is selective for a first polypeptide comprising the sequence TAKILE (SEQ ID No:204, NAV1.8 motif) over a second polypeptide comprising the sequence VANWLG (SEQ ID No:272, NAV1.4 motif);
q. is selective for a first polypeptide comprising the sequence SEVAP (SEQ ID No:264, NAV1.8 motif) over a second polypeptide comprising the sequence SELGP (SEQ ID No:273, NAV1.4 motif);
r. is selective for a first polypeptide comprising the sequence SLTAKI (SEQ ID No:265, NAV1.8 motif) over a second polypeptide comprising the sequence SLVANW (SEQ ID No:195, NAV1.4 motif);
s. is selective for a first polypeptide comprising the sequence KILEY (SEQ ID No:266, NAV1.8 motif) over a second polypeptide comprising the sequence NWLGY (SEQ ID No:274, NAV1.4 motif);
t. is selective for a first polypeptide comprising the sequence ISLTAKILEYSEVAPIK (SEQ ID No:30, NAV1.8 motif) over a second polypeptide comprising the sequence ISLVANWLGYSELGPIK (SEQ ID No:120, NAV1.4 motif);
u. is selective for a first polypeptide comprising the sequence TAKILE (SEQ ID No:204, NAV1.8 motif) over a second polypeptide comprising the sequence VANTLG (SEQ ID No:203, NAV1.5 and NAV1.7 motif);
v. is selective for a first polypeptide comprising the sequence SEVAP (SEQ ID No:264, NAV1.8 motif) over a second polypeptide comprising the sequence AEMGP (SEQ ID No:276, NAV1.5 motif);
w. is selective for a first polypeptide comprising the sequence SLTAKI (SEQ ID No:265, NAV1.8 motif) over a second polypeptide comprising the sequence SLVANT (SEQ ID No:277, NAV1.5 motif);
x. is selective for a first polypeptide comprising the sequence KILEY (SEQ ID No:266, NAV1.8 motif) over a second polypeptide comprising the sequence ANTLGF (SEQ ID No:278, NAV1.5 motif);
y. is selective for a first polypeptide comprising the sequence ISLTAKILEYSEVAPIK (SEQ ID No:30, NAV1.8 motif) over a second polypeptide comprising the sequence VSLVANTLGFAEMGPIK (SEQ ID No:138, NAV1.5 motif);
z. is selective for a first polypeptide comprising the sequence ISLTA (SEQ ID No:202, NAV1.8 motif) over a second polypeptide comprising the sequence VTLVA (SEQ ID No:193, NAV1.7 motif);
aa. is selective for a first polypeptide comprising the sequence SEVAP (SEQ ID No:264, NAV1.8 motif) over a second polypeptide comprising the sequence SDLGP (SEQ ID No:187, NAV1.7 motif);
bb. is selective for a first polypeptide comprising the sequence SLTAKI (SEQ ID No:265, NAV1.8 motif) over a second polypeptide comprising the sequence TLVANT (SEQ ID No:185, NAV1.7 motif);
cc. is selective for a first polypeptide comprising the sequence KILEY (SEQ ID No:266, NAV1.8 motif) over a second polypeptide comprising the sequence ANTLGY (SEQ ID No:279, NAV1.7 motif);
dd. is selective for a first polypeptide comprising the sequence ISLTAKILEYSEVAPIK (SEQ ID No:30, NAV1.8 motif) over a second polypeptide comprising the sequence VTLVANTLGYSDLGPIK (SEQ ID No:12, NAV1.7 motif);
ee. is selective for a first polypeptide comprising the sequence ISLTA (SEQ ID No:202, NAV1.8 motif) over a second polypeptide comprising the sequence TTLIN (SEQ ID No:207, NAV1.9 motif);
ff. is selective for a first polypeptide comprising the sequence YSEV (SEQ ID No:280, NAV1.8 motif) over a second polypeptide comprising the sequence LMEL (SEQ ID No:209, NAV1.9 motif); and
gg. is selective for a first polypeptide comprising the sequence ISLTAKILEYSEVAPIK (SEQ ID No:30, NAV1.8 motif) over a second polypeptide comprising the sequence TTLINLMELK (SEQ ID No:48, NAV1.9 motif).

Sentence 193. An antibody or fragment according to:
n. Sentence 192a, 192b, 192c, 192d, 192e or sentence 192f, wherein the second polypeptide is a human NAV1.6 protein; or
o. Sentence 192c, 192e, 192g, 192h, 192i or sentence 192j, wherein the second polypeptide is a human NAV1.1 protein or a human NAV1.2 protein; or
p. Sentence 192c, 192e, 192k, 192l, 192m or sentence 192n, wherein the second polypeptide is a human NAV1.3 protein; or
q. Sentence 192o, 192p, 192q, 192r, 192s or sentence 192t, wherein the second polypeptide is a human NAV1.4 protein; or
r. Sentence 192k, 192u, 192v, 192w, 192x or sentence 192y, wherein the second polypeptide is a human NAV1.5 protein; or
s. Sentence 192u, 192z, 192aa, 192bb, 192cc or sentence 192dd, wherein the second polypeptide is a human NAV1.7 protein; or
t. Sentence 192ee, 192ff or sentence 192gg, wherein the second polypeptide is a human NAV1.9 protein.

Sentence 194. An antibody or fragment according to sentence 192 or sentence 193 which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 195. An antibody or fragment according to sentence 194, wherein the antibody or fragment binds to the D3E2 loop (SEQ ID No:30) of human NAV1.8.

Sentence 196. An antibody or fragment according to any one of sentences 192 to 195, wherein the first polypeptide is a NAV1.8 protein.

Sentence 197. An antibody or fragment according to any one of sentences 192 to 196, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 198. An antibody or fragment thereof according to any one of sentences 192 to 197, which has one, more (e.g. 2, 3, 4 or 5) or all of the features of sentence 192a, sentence 192b, sentence 192c, sentence 192d, sentence 192e and sentence 192f; and one more (e.g. 2, 3, 4 or 5) or all of the features of sentence 192o, sentence 192p, sentence 192q, sentence 192r, sentence 192s and sentence 192t.

Sentence 199. An antibody or fragment thereof according to any one of sentences 192 to 197, which has one, more (e.g. 2, 3, 4 or 5) or all of the features of sentence 192a, sentence 192b, sentence 192c, sentence 192d, sentence 192e and sentence 192f; and one, more (e.g. 2, 3, 4 or 5) or all of the features of sentence 192k, sentence 192u, sentence 192v, sentence 192w, sentence 192x and sentence 192y.

Sentence 200. An antibody or fragment thereof according to any one of sentences 192 to 197, which has one, more (e.g. 2, 3, 4 or 5) or all of the features of sentence 192o, sentence 192p, sentence 192q, sentence 192r, sentence 192s and sentence 192t; and one, more (e.g. 2, 3, 4 or 5) or all of the features of sentence 192k, sentence 192u, sentence 192v, sentence 192w, sentence 192x and sentence 192y.

Sentence 201. An antibody or fragment thereof according to any one of sentences 192 to 197, which has one, more (e.g. 2, 3, 4 or 5) or all of the features of sentence 192o, sentence 192p, sentence 192q, sentence 192r, sentence 192s and sentence 192t; and one, more (e.g. 2, 3, 4 or 5) or all of the features of sentence 192k, sentence 192u, sentence 192v, sentence 192w, sentence 192x and sentence 192y; and one, more (e.g. 2, 3, 4 or 5) or all of the features of sentence 192a, sentence 192b, sentence 192c, sentence 192d, sentence 192e and sentence 192f.

Sentence 209. An antibody or fragment thereof which binds to human NAV1.8 (SEQ ID No:20), and comprises one, more (e.g. 2, 3, 4 or 5), or all of the features a. to u.:
a. is selective for a first polypeptide comprising the sequence ASLIFSA (SEQ ID No:282, NAV1.8 motif) over a second polypeptide comprising the sequence VGMFLAD (SEQ ID No:283, NAV1.6 and NAV1.7 motif);
b. is selective for a first polypeptide comprising the sequence IFSAILK (SEQ ID No:284, NAV1.8 motif) over a second polypeptide comprising the sequence FLADIIE (SEQ ID No:285, NAV1.6 motif);
c. is selective for a first polypeptide comprising the sequence ASLIFSAILKSLQSYFSPTLFR (SEQ ID No:34, NAV1.8 motif) over a second polypeptide comprising the sequence VGMFLADIIEKYFVSPTLFR (SEQ ID No:160, NAV1.6 motif);
d. is selective for a first polypeptide comprising the sequence ASLIFSA (SEQ ID No:282, NAV1.8 motif) over a second polypeptide comprising the sequence VGMFLAE (SEQ ID No:286, NAV1.1, NAV1.2 and NAV1.3 motif);
e. is selective for a first polypeptide comprising the sequence IFSAILK (SEQ ID No:284, NAV1.8 motif) over a second polypeptide comprising the sequence FLAELIE (SEQ ID No:287, NAV1.1 and NAV1.2 motif);
f. is selective for a first polypeptide comprising the sequence ASLIFSAILKSLQSYFSPTLFR (SEQ ID No:34, NAV1.8 motif) over a second polypeptide comprising the sequence VGMFLAELIEKYFVSPTLFR (SEQ ID No:70, NAV1.1 and NAV1.2 motif);
g. is selective for a first polypeptide comprising the sequence IFSAILK (SEQ ID No:284, NAV1.8 motif) over a second polypeptide comprising the sequence FLAEMIE (SEQ ID No:288, NAV1.3 motif);
h. is selective for a first polypeptide comprising the sequence ASLIFSAILKSLQSYFSPTLFR (SEQ ID No:34, NAV1.8 motif) over a second polypeptide comprising the sequence VGMFLAEMIEKYFVSPTLFR (SEQ ID No:106, NAV1.3 motif);
i. is selective for a first polypeptide comprising the sequence ASLIFSA (SEQ ID No:282, NAV1.8 motif) over a second polypeptide comprising the sequence VGLALSD (SEQ ID No:289, NAV1.4 motif);
j. is selective for a first polypeptide comprising the sequence IFSAILK (SEQ ID No:284, NAV1.8 motif) over a second polypeptide comprising the sequence ALSDLIQ (SEQ ID No:290, NAV1.4 motif);
k. is selective for a first polypeptide comprising the sequence ASLIFSAILKSLQSYFSPTLFR (SEQ ID No:34, NAV1.8 motif) over a second polypeptide comprising the sequence VGLALSDLIQKYFVSPTLFR (SEQ ID No:124, NAV1.4 motif);
l. is selective for a first polypeptide comprising the sequence ASLIFSA (SEQ ID No:282, NAV1.8 motif) over a second polypeptide comprising the sequence VGTVLSD (SEQ ID No:291, NAV1.5 motif);
m. is selective for a first polypeptide comprising the sequence IFSAILK (SEQ ID No:284, NAV1.8 motif) over a second polypeptide comprising the sequence VLSDIIQ (SEQ ID No:292, NAV1.5 motif);
n. is selective for a first polypeptide comprising the sequence ASLIFSAILKSLQSYFSPTLFR (SEQ ID No:34, NAV1.8 motif) over a second polypeptide comprising the sequence VGTVLSDIIQKYFFSPTLFR (SEQ ID No:142, NAV1.5 motif);
o. is selective for a first polypeptide comprising the sequence IFSAILK (SEQ ID No:284, NAV1.8 motif) over a second polypeptide comprising the sequence FLADLIE (SEQ ID No:293, NAV1.7 motif);
p. is selective for a first polypeptide comprising the sequence QSYFSP (SEQ ID No:296, NAV1.8 motif) over a second polypeptide comprising the sequence TYFVSP (SEQ ID No:318, NAV1.7 motif);
q. is selective for a first polypeptide comprising the sequence ASLIFSAILKSLQSYFSPTLFR (SEQ ID No:34, NAV1.8 motif) over a second polypeptide comprising the sequence VGMFLADLIETYFVSPTLFR (SEQ ID No:16, NAV1.7 motif);
r. is selective for a first polypeptide comprising the sequence ASLIFSA (SEQ ID No:282, NAV1.8 motif) over a second polypeptide comprising the sequence VSTMIST (SEQ ID No:294, NAV1.9 motif);
s. is selective for a first polypeptide comprising the sequence IFSAILK (SEQ ID No:284, NAV1.8 motif) over a second polypeptide comprising the sequence MISTLEN (SEQ ID No:295, NAV1.9 motif);
t. is selective for a first polypeptide comprising the sequence QSYFSP (SEQ ID No:296, NAV1.8 motif) over a second polypeptide comprising the sequence HIPFPP (SEQ ID No:297, NAV1.9 motif); and
u. is selective for a first polypeptide comprising the sequence ASLIFSAILKSLQSYFSPTLFR (SEQ ID No:34, NAV1.8 motif) over a second polypeptide comprising the sequence VSTMISTLENQEHIPFPPTLFR (SEQ ID No:52, NAV1.9 motif).

Sentence 210. An antibody or fragment according to:
u. Sentence 209a, 209b or sentence 209c, wherein the second polypeptide is a human NAV1.6 protein; or
v. Sentence 209d, 209e or sentence 209f, wherein the second polypeptide is a human NAV1.1 protein or a human NAV1.2 protein; or
w. Sentence 209d, 209g or sentence 209h, wherein the second polypeptide is a human NAV1.3 protein; or
x. Sentence 209i, 209j or sentence 209k, wherein the second polypeptide is a human NAV1.4 protein; or
y. Sentence 209l, 209m or sentence 209n, wherein the second polypeptide is a human NAV1.5 protein; or
z. Sentence 209a, 209o, 209p or sentence 209q, wherein the second polypeptide is a human NAV1.7 protein; or
aa. Sentence 209r, 209s, 209t or sentence 209u, wherein the second polypeptide is a human NAV1.9 protein.

Sentence 211. An antibody or fragment according to sentence 209 or sentence 210 which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 212. An antibody or fragment according to sentence 211, wherein the antibody or fragment binds to the D4E2 loop (SEQ ID No:34) of human NAV1.8.

Sentence 213. An antibody or fragment according to any one of sentences 209 to 212, wherein the first polypeptide is a NAV1.8 protein.

Sentence 214. An antibody or fragment according to any one of sentences 209 to 213, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 215. An antibody or fragment thereof according to any one of sentences 209 to 214, which has one, two or all of the features of sentence 209a, sentence 209b and sentence 209c; and one, two or all of the features of sentence 209l, sentence 209j and sentence 209k.

Sentence 216. An antibody or fragment thereof according to any one of sentences 209 to 214, which has one, two or all of the features of sentence 209a, sentence 209b and sentence 209c; and one, two or all of the features of sentence 209l sentence 209m and sentence 209n.

Sentence 217. An antibody or fragment thereof according to any one of sentences 209 to 214, which has the features of sentence 126i, sentence 126j and/or sentence 126k, and the features of sentence 126l sentence 126m and/or sentence 126n.

Sentence 218. An antibody or fragment thereof according to any one of sentences 209 to 214, which has one, two of all of the features of sentence 209i, sentence 209j and sentence 209k; and one, two or all of the features of sentence 209l, sentence 209m and sentence 209n; and one, two or all of the features of sentence 209a, sentence 209b and sentence 209c.

Sentence 226. An antibody or fragment thereof which specifically binds to human NAV1.9 (SEQ ID No:38), and comprises one, more (e.g. 2, 3, 4 or 5), or all of the features a. to q.:
a. is selective for a first polypeptide comprising the sequence VSYIP (SEQ ID No:298, NAV1.9 motif) over a second polypeptide comprising the sequence ITEFV (SEQ ID No:299, NAV1.6 motif);
b. is selective for a first polypeptide comprising the sequence YIPGI (SEQ ID No:301, NAV1.9 motif) over a second polypeptide comprising the sequence EFVNL (SEQ ID No:300, NAV1.6 and NAV1.7 motif);
c. is selective for a first polypeptide comprising the sequence GMK (SEQ ID No:302, NAV1.9 motif) over a second polypeptide comprising the sequence VNLGN (SEQ ID No:237, NAV1.6 and NAV1.7 motif);
d. is selective for a first polypeptide comprising the sequence VSYIPGITIK (SEQ ID No:40, NAV1.9 motif) over a second polypeptide comprising the sequence ITEFVNLGN (SEQ ID No:148, NAV1.6 motif);
e. is selective for a first polypeptide comprising the sequence VSYIP (SEQ ID No:298, NAV1.9 motif) over a second polypeptide comprising the sequence VTEFV (SEQ ID No:303, NAV1.1, NAV1.2 and NAV1.3 motif);
f. is selective for a first polypeptide comprising the sequence YIPGI (SEQ ID No:301, NAV1.9 motif) over a second polypeptide comprising the sequence EFVDL (SEQ ID No:304, NAV1.1, NAV1.2, NAV1.3, NAV1.4 and NAV1.5 motif);
g. is selective for a first polypeptide comprising the sequence GITIK (SEQ ID No:302, NAV1.9 motif) over a second polypeptide comprising the sequence VDLGN (SEQ ID No:238, NAV1.1, NAV1.2, NAV1.3, NAV1.4 and NAV1.5 motif);
h. is selective for a first polypeptide comprising the sequence VSYIPGITIK (SEQ ID No:40, NAV1.9 motif) over a second polypeptide comprising the sequence VTEFVDLGN (SEQ ID No:58, NAV1.1, NAV1.2 and NAV1.3 motif);
i. is selective for a first polypeptide comprising the sequence VSYIP (SEQ ID No:298, NAV1.9 motif) over a second polypeptide comprising the sequence LTEFV (SEQ ID No:305, NAV1.4 and NAV1.7 motif);
j. is selective for a first polypeptide comprising the sequence VSYIPGITIK (SEQ ID No:40, NAV1.9 motif) over a second polypeptide comprising the sequence LTEFVDLGN (SEQ ID No:112, NAV1.4 motif);
k. is selective for a first polypeptide comprising the sequence VSYIP (SEQ ID No:298, NAV1.9 motif) over a second polypeptide comprising the sequence TTEFV (SEQ ID No:306, NAV1.5 motif);
l. is selective for a first polypeptide comprising the sequence VSYIPGITIK (SEQ ID No:40, NAV1.9 motif) over a second polypeptide comprising the sequence TTEFVDLGN (SEQ ID No:130, NAV1.5 motif);
m. is selective for a first polypeptide comprising the sequence VSYIPGITIK (SEQ ID No:40, NAV1.9 motif) over a second r polypeptide comprising the sequence LTEFVNLGN (SEQ ID No:4, NAV1.7 motif);
n. is selective for a first polypeptide comprising the sequence VSYIP (SEQ ID No:298, NAV1.9 motif) over a second polypeptide comprising the sequence VGTAI (SEQ ID No:307, NAV1.7 motif);
o. is selective for a first polypeptide comprising the sequence YIPGI (SEQ ID No:301, NAV1.9 motif) over a second polypeptide comprising the sequence TAIDL (SEQ ID No:196, NAV1.8 motif);
p. is selective for a first polypeptide comprising the sequence GITIK (SEQ ID No:302, NAV1.9 motif) over a second polypeptide comprising the sequence IDLRG (SEQ ID No:236, NAV1.8 motif); and
q. is selective for a first polypeptide comprising the sequence VSYIPGITIK (SEQ ID No:40, NAV1.9 motif) over a second polypeptide comprising the sequence VGTAIDLRG (SEQ ID No:22, NAV1.8 motif).

Sentence 227. An antibody or fragment according to:
a. Sentence 226a, 226b, 226c or sentence 226d, wherein the second polypeptide is a human NAV1.6 protein; or
b. Sentence 226e, 226f, 226g or sentence 226h, wherein the second polypeptide is a human NAV1.1 protein, or a human NAV1.2 protein or a human NAV1.3 protein; or
c. Sentence 226f, 226g, 226i or sentence 226j, wherein the second polypeptide is a human NAV1.4 protein; or
d. Sentence 226f, 226g, 226k or sentence 226l, wherein the second polypeptide is a human NAV1.5 protein; or
e. Sentence 226b, 226c, 226i, 226m or sentence 226n, wherein the second polypeptide is a human NAV1.7 protein; or
f. Sentence 226o, 226p or sentence 226q, wherein the second polypeptide is a human NAV1.8 protein.

Sentence 228. An antibody or fragment according to sentence 226 or sentence 227 which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 229. An antibody or fragment according to sentence 228, wherein the antibody or fragment binds to the D1E2 loop (SEQ ID No:40) of human NAV1.9.

Sentence 230. An antibody or fragment according to any one of sentences 226 to 229, wherein the first polypeptide is a NAV1.9 protein.

Sentence 231. An antibody or fragment according to any one of sentences 226 to 230, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 232. An antibody or fragment thereof according to any one of sentences 226 to 231, which has the features of sentence 132a, sentence 132b, sentence 132c and/or sentence 132d, and the features of sentence 132i and/or sentence 132j.

Sentence 233. An antibody or fragment thereof according to any one of sentences 226 to 231, which has one, more (e.g. 2 or 3) or all of the features of sentence 226a, sentence 226b, sentence 226c and sentence 226d; and one or both of the features of sentence 226k and sentence 226l.

Sentence 234. An antibody or fragment thereof according to any one of sentences 226 to 231, which
a. has the features of sentence 226g; or
b. has the features of sentence 226f; or
c. has one or both of the features of sentence 226i and sentence 226j; and one or both of the features of sentence 226k and sentence 226l.

Sentence 235. An antibody or fragment thereof according to any one of sentences 226 to 231, which has one or both of the features of sentence 226i and sentence 226j; and one or both of the features of sentence 226k and sentence 226l; and one, more (e.g. 2 or 3) or all of the features of sentence 226a, sentence 226b, sentence 226c and sentence 226d.

Sentence 243. An antibody or fragment thereof which binds to human NAV1.9 (SEQ ID No:38), and comprises one, more (e.g. 2, 3, 4 or 5), or all of the features a. to s.:
a. is selective for a first polypeptide comprising the sequence ADVM (SEQ ID No:76, NAV1.9 motif) over a second polypeptide comprising the sequence MELS (SEQ ID No:177, NAV1.6 motif);
b. is selective for a first polypeptide comprising the sequence VLQKRS (SEQ ID No:261, NAV1.9 motif) over a second polypeptide comprising the sequence LADVEG (SEQ ID No:243, NAV1.6 and NAV1.7 motif);
c. is selective for a first polypeptide comprising the sequence ADVMNCVLQKRS (SEQ ID No:44, NAV1.9 motif) over a second polypeptide comprising the sequence MELSLADVEG (SEQ ID No:152, NAV1.6 motif);
d. is selective for a first polypeptide comprising the sequence ADVM (SEQ ID No:76, NAV1.9 motif) over a second polypeptide comprising the sequence VELG (SEQ ID No:88, NAV1.1 and NAV1.4 motif);
e. is selective for a first polypeptide comprising the sequence VLQKRS (SEQ ID No:261, NAV1.9 motif) over a second polypeptide comprising the sequence LANVEG (SEQ ID No:248, NAV1.1 and NAV1.2 motif);
f. is selective for a first polypeptide comprising the sequence ADVMNCVLQKRS (SEQ ID No:44, NAV1.9 motif) over a second polypeptide comprising the sequence VELGLANVEG (SEQ ID No:62, NAV1.1 motif);
g. is selective for a first polypeptide comprising the sequence ADVM (SEQ ID No:76, NAV1.9 motif) over a second polypeptide comprising the sequence MELG (SEQ ID No:253, NAV1.2, NAV1.3 and NAV1.5 motif);
h. is selective for a first polypeptide comprising the sequence ADVMNCVLQKRS (SEQ ID No:44, NAV1.9 motif) over a second polypeptide comprising the sequence MELGLANVEG (SEQ ID No:80, NAV1.2 motif);
i. is selective for a first polypeptide comprising the sequence VLQKRS (SEQ ID No:261, NAV1.9 motif) over a second polypeptide comprising the sequence LSNVEG (SEQ ID No:254, NAV1.3 motif);
j. is selective for a first polypeptide comprising the sequence ADVMNCVLQKRS (SEQ ID No:44, NAV1.9 motif) over a second polypeptide comprising the sequence MELGLSNVEG (SEQ ID No:98, NAV1.3 motif);
k. is selective for a first polypeptide comprising the sequence VLQKRS (SEQ ID No:261, NAV1.9 motif) over a second polypeptide comprising the sequence LANVQG (SEQ ID No:256, NAV1.4 motif);
l. is selective for a first polypeptide comprising the sequence ADVMNCVLQKRS (SEQ ID No:44, NAV1.9 motif) over a second polypeptide comprising the sequence VELGLANVQG (SEQ ID No:116, NAV1.4 motif);
m. is selective for a first polypeptide comprising the sequence VLQKRS (SEQ ID No:261, NAV1.9 motif) over a second polypeptide comprising the sequence LSRMSN (SEQ ID No:258, NAV1.5 motif);
n. is selective for a first polypeptide comprising the sequence ADVMNCVLQKRS (SEQ ID No:44, NAV1.9 motif) over a second polypeptide comprising the sequence MELGLSRMSN (SEQ ID No:134, NAV1.5 motif);
o. is selective for a first polypeptide comprising the sequence ADVM (SEQ ID No:76, NAV1.9 motif) over a second polypeptide comprising the sequence VELF (SEQ ID No:176, NAV1.7 motif);
p. is selective for a first polypeptide comprising the sequence ADVMNCVLQKRS (SEQ ID No:44, NAV1.9 motif) over a second polypeptide comprising the sequence VELFLANVEG (SEQ ID No:8, NAV1.7 motif);
q. is selective for a first polypeptide comprising the sequence ADVM (SEQ ID No:76, NAV1.9 motif) over a second polypeptide comprising the sequence LELG (SEQ ID No:241, NAV1.8 motif);
r. is selective for a first polypeptide comprising the sequence VLQKRS (SEQ ID No:261, NAV1.9 motif) over a second polypeptide comprising the sequence VAKKGS (SEQ ID No:242, NAV1.8 motif); and
s. is selective for a first polypeptide comprising the sequence ADVMNCVLQKRS (SEQ ID No:44, NAV1.9 motif) over a second polypeptide comprising the sequence LELGVAKKGS (SEQ ID No:26, NAV1.8 motif).

Sentence 244. An antibody or fragment according to:
a. Sentence 244a, 244b or sentence 244c, wherein the second polypeptide is a human NAV1.6 protein; or
b. Sentence 244d, 244e or sentence 244f, wherein the second polypeptide is a human NAV1.1 protein; or
c. Sentence 244e, 244g or sentence 244h, wherein the second polypeptide is a human NAV1.2 protein; or
d. Sentence 244g, 244i or sentence 244j, wherein the second polypeptide is a human NAV1.3 protein; or
e. Sentence 244d, 244k or sentence 244l, wherein the second polypeptide is a human NAV1.4 protein; or
f. Sentence 244g, 244m or sentence 244n, wherein the second polypeptide is a human NAV1.5 protein; or
g. Sentence 244b, 244o or sentence 244p, wherein the second polypeptide is a human NAV1.7 protein; or
h. Sentence 244q, 244r or sentence 244s, wherein the second polypeptide is a human NAV1.8 protein.

Sentence 245. An antibody or fragment according to sentence 244 or sentence 245 which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 246. An antibody or fragment according to sentence 245, wherein the antibody or fragment binds to the D2E2 loop (SEQ ID No:44) of human NAV1.9.

Sentence 247. An antibody or fragment according to any one of sentences 244 to 246, wherein the first polypeptide is a NAV1.9 protein.

Sentence 248. An antibody or fragment according to any one of sentences 244 to 247, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 249. An antibody or fragment thereof according to any one of sentences 244 to 248, which has one, two or all of the features of sentence 244a, sentence 244b and sentence 244c; and one, two or all of the features of sentence 244d, sentence 244k and sentence 244l.

Sentence 250. An antibody or fragment thereof according to any one of sentences 244 to 248, which has one, two or all of the features of sentence 244a, sentence 244b and sentence 244c; and one, two or all of the features of sentence 244g, sentence 244m and sentence 244n.

Sentence 251. An antibody or fragment thereof according to any one of sentences 244 to 248, which has one, two or all of the features of sentence 244d, sentence 244k and sentence 244l; and one, two or all of the features of sentence 244g, sentence 244m and sentence 244n.

Sentence 252. An antibody or fragment thereof according to any one of sentences 244 to 248, which has one, two or all of the features of sentence 244d, sentence 244k and sentence 244l; and one, two or all of the features of sentence 244g, sentence 244m and sentence 244n; and one, two or all of the features of sentence 244a, sentence 244b and sentence 244c.

Sentence 260. An antibody or fragment thereof which specifically binds to human NAV1.9 (SEQ ID No:38), and comprises one, more (e.g. 2, 3, 4 or 5), or all of the features a. to q.:
a. is selective for a first polypeptide comprising the sequence TTLIN (SEQ ID No:207, NAV1.9 motif) over a second polypeptide comprising the sequence VSLIA (SEQ ID No:262, NAV1.6 motif);
b. is selective for a first polypeptide comprising the sequence LMELK (SEQ ID No:275, NAV1.9 motif) over a second polypeptide comprising the sequence LGAIK (SEQ ID No:281, NAV1.6, NAV1.1., NAV1.2 and NAV1.3 motif);
c. is selective for a first polypeptide comprising the sequence TTLINLMELK (SEQ ID No:48, NAV1.9 motif) over a second polypeptide comprising the sequence VSLIANALGYSELGAIK (SEQ ID No:156, NAV1.6 motif);
d. is selective for a first polypeptide comprising the sequence TTLIN (SEQ ID No:207, NAV1.9 motif) over a second polypeptide comprising the sequence VSLTA (SEQ ID No:268, NAV1.1 and NAV1.2 motif);
e. is selective for a first polypeptide comprising the sequence TTLINLMELK (SEQ ID No:48, NAV1.9 motif) over a second polypeptide comprising the sequence VSLTANALGYSELGAIK (SEQ ID No:66, NAV1.1 and NAV1.2 motif);
f. is selective for a first polypeptide comprising the sequence TTLIN (SEQ ID No:207, NAV1.9 motif) over a second polypeptide comprising the sequence VSLVA (SEQ ID No:270, NAV1.3 and NAV1.5 motif);
g. is selective for a first polypeptide comprising the sequence TTLINLMELK (SEQ ID No:48, NAV1.9 motif) over a second polypeptide comprising the sequence VSLVANALGYSELGAIK (SEQ ID No:102, NAV1.3 motif);
h. is selective for a first polypeptide comprising the sequence TTLIN (SEQ ID No:207, NAV1.9 motif) over a second polypeptide comprising the sequence ISLVA (SEQ ID No:194, NAV1.4 motif);
i. is selective for a first polypeptide comprising the sequence LMELK (SEQ ID No:275, NAV1.9 motif) over a second polypeptide comprising the sequence LGPIK (SEQ ID No:308, NAV1.4 and NAV1.7 motif);
j. is selective for a first polypeptide comprising the sequence TTLINLMELK (SEQ ID No:48, NAV1.9 motif) over a second polypeptide comprising the sequence ISLVANWLGYSELGPIK (SEQ ID No:120, NAV1.4 motif);
k. is selective for a first polypeptide comprising the sequence LMELK (SEQ ID No:275, NAV1.9 motif) over a second polypeptide comprising the sequence MGPIK (SEQ ID No:309, NAV1.5 motif);
l. is selective for a first polypeptide comprising the sequence TTLINLMELK (SEQ ID No:48, NAV1.9 motif) over a second polypeptide comprising the sequence VSLVANTLGFAEMGPIK (SEQ ID No:138, NAV1.5 motif);
m. is selective for a first polypeptide comprising the sequence TTLIN (SEQ ID No:207, NAV1.9 motif) over a second polypeptide comprising the sequence VTLVA (SEQ ID No:193, NAV1.7 motif);
n. is selective for a first polypeptide comprising the sequence TTLINLMELK (SEQ ID No:48, NAV1.9 motif) over a second polypeptide comprising the sequence VTLVANTLGYSDLGPIK (SEQ ID No:12, NAV1.7 motif);
o. is selective for a first polypeptide comprising the sequence TTLIN (SEQ ID No:207, NAV1.9 motif) over a second polypeptide comprising the sequence ISLTA (SEQ ID No:202, NAV1.8 motif);
p. is selective for a first polypeptide comprising the sequence LMELK (SEQ ID No:275, NAV1.9 motif) over a second polypeptide comprising the sequence VAPIK (SEQ ID No:310, NAV1.8 motif); and
q. is selective for a first polypeptide comprising the sequence TTLINLMELK (SEQ ID No:48, NAV1.9 motif) over a second polypeptide comprising the sequence ISLTAKILEYSEVAPIK (SEQ ID No:30, NAV1.8 motif).

Sentence 261. An antibody or fragment according to:
a. Sentence 260a, 260b or sentence 260c, wherein the second polypeptide is a human NAV1.6 protein; or
b. Sentence 260b, 260d or sentence 260e, wherein the second polypeptide is a human NAV1.1 protein or a human NAV1.2 protein; or
c. Sentence 260b, 260f or sentence 260g, wherein the second polypeptide is a human NAV1.3 protein; or
d. Sentence 260h, 260i or sentence 260j, wherein the second polypeptide is a human NAV1.4 protein; or
e. Sentence 260f, 260k or sentence 260l, wherein the second polypeptide is a human NAV1.5 protein; or
f. Sentence 260i, 260m or sentence 260n, wherein the second polypeptide is a human NAV1.7 protein; or
g. Sentence 260o, 260p or sentence 260q, wherein the second polypeptide is a human NAV1.8 protein.

Sentence 262. An antibody or fragment according to sentence 260 or sentence 261 which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 263. An antibody or fragment according to sentence 262, wherein the antibody or fragment binds to the D3E2 loop (SEQ ID No:48) of human NAV1.9.

Sentence 264. An antibody or fragment according to any one of sentences 260 to 263, wherein the first polypeptide is a NAV1.9 protein.

Sentence 265. An antibody or fragment according to any one of sentences 260 to 264, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 266. An antibody or fragment thereof according to any one of sentences 260 to 265, which has one, two or all of the features of sentence 260a, sentence 260b and sentence 260c; and one, two or all of the features of sentence 260h, sentence 260i and sentence 260j.

Sentence 267. An antibody or fragment thereof according to any one of sentences 260 to 265, which has one, two or all of the features of sentence 260a, sentence 260b and sentence 260c; and one, two or all of the features of sentence 260f, sentence 260k and sentence 260l.

Sentence 268. An antibody or fragment thereof according to any one of sentences 260 to 265, which has one, two or all of the features of sentence 260h, sentence 260i and sentence 260j; and one, two or all of the features of sentence 260f, sentence 260k and sentence 260l.

Sentence 269. An antibody or fragment thereof according to any one of sentences 260 to 265, which has one, two or all of the features of sentence 260h, sentence 260i and sentence 260j; and one, two or all of the features of sentence 260f, sentence 260k and sentence 260l; and one, two or all of the features of sentence 260a, sentence 260b and sentence 260c.

Sentence 277. An antibody or fragment thereof which binds to human NAV1.9 (SEQ ID No:38), and comprises one, more (e.g. 2, 3, 4 or 5), or all of the features a. to dd.:
a. is selective for a first polypeptide comprising the sequence HIPFPP (SEQ ID No:297, NAV1.9 motif) over a second polypeptide comprising the sequence KYFVSP (SEQ ID No:311, NAV1.6, NAV1.1, NAV1.2, NAV1.3 and NAV1.4 motif);
b. is selective for a first polypeptide comprising the sequence VSTMIST (SEQ ID No:294, NAV1.9 motif) over a second polypeptide comprising the sequence VGMFLAD (SEQ ID No:283, NAV1.6 and NAV1.7 motif);
c. is selective for a first polypeptide comprising the sequence MISTLEN (SEQ ID No:295, NAV1.9 motif) over a second polypeptide comprising the sequence FLADIIE (SEQ ID No:285, NAV1.6 motif);
d. is selective for a first polypeptide comprising the sequence STMISTLEN (SEQ ID No:312, NAV1.9 motif) over a second polypeptide comprising the sequence GMFLADIIE (SEQ ID No:313, NAV1.6 motif);
e. is selective for a first polypeptide comprising the sequence VSTMISTLENQEHIPFPPTLFR (SEQ ID No:52, NAV1.9 motif) over a second polypeptide comprising the sequence VGMFLADIIEKYFVSPTLFR (SEQ ID No:160, NAV1.6 motif);
f. is selective for a first polypeptide comprising the sequence VSTMIST (SEQ ID No:294, NAV1.9 motif) over a second polypeptide comprising the sequence VGMFLAE (SEQ ID No:286, NAV1.1, NAV1.2 and NAV1.3 motif);
g. is selective for a first polypeptide comprising the sequence MISTLEN (SEQ ID No:295, NAV1.9 motif) over a second polypeptide comprising the sequence FLAELIE (SEQ ID No:287, NAV1.1 and NAV1.2 motif);
h. is selective for a first polypeptide comprising the sequence STMISTLEN (SEQ ID No:312, NAV1.9 motif) over a second polypeptide comprising the sequence GMFLAELIE (SEQ ID No:314, NAV1.1 and NAV1.2 motif);
i. is selective for a first polypeptide comprising the sequence VSTMISTLENQEHIPFPPTLFR (SEQ ID No:52, NAV1.9 motif) over a second polypeptide comprising the sequence VGMFLAELIEKYFVSPTLFR (SEQ ID No:70, NAV1.1 and NAV1.2 motif);
j. is selective for a first polypeptide comprising the sequence MISTLEN (SEQ ID No:295, NAV1.9 motif) over a second polypeptide comprising the sequence FLAEMIE (SEQ ID No:288, NAV1.3 motif);
k. is selective for a first polypeptide comprising the sequence STMISTLEN (SEQ ID No:312, NAV1.9 motif) over a second polypeptide comprising the sequence GMFLAEMIE (SEQ ID No:315, NAV1.3 motif);
l. is selective for a first polypeptide comprising the sequence VSTMISTLENQEHIPFPPTLFR (SEQ ID No:52, NAV1.9 motif) over a second polypeptide comprising the sequence VGMFLAEMIEKYFVSPTLFR (SEQ ID No:106, NAV1.3 motif);
m. is selective for a first polypeptide comprising the sequence VSTMIST (SEQ ID No:294, NAV1.9 motif) over a second polypeptide comprising the sequence VGLALSD (SEQ ID No:289, NAV1.4 motif);
n. is selective for a first polypeptide comprising the sequence MISTLEN (SEQ ID No:295, NAV1.9 motif) over a second polypeptide comprising the sequence ALSDLIQ (SEQ ID No:290, NAV1.4 motif);
o. is selective for a first polypeptide comprising the sequence STMISTLEN (SEQ ID No:312, NAV1.9 motif) over a second polypeptide comprising the sequence GLALSDLIQ (SEQ ID No:322, NAV1.4 motif);
p. is selective for a first polypeptide comprising the sequence VSTMISTLENQEHIPFPPTLFR (SEQ ID No:52, NAV1.9 motif) over a second polypeptide comprising the sequence VGLALSDLIQKYFVSPTLFR (SEQ ID No:124, NAV1.4 motif);
q. is selective for a first polypeptide comprising the sequence HIPFPP (SEQ ID No:297, NAV1.9 motif) over a second polypeptide comprising the sequence KYFFSP (SEQ ID No:316, NAV1.5 motif);
r. is selective for a first polypeptide comprising the sequence VSTMIST (SEQ ID No:294, NAV1.9 motif) over a second polypeptide comprising the sequence VGTVLSD (SEQ ID No:291, NAV1.5 motif);
s. is selective for a first polypeptide comprising the sequence MISTLEN (SEQ ID No:295, NAV1.9 motif) over a second polypeptide comprising the sequence VLSDIIQ (SEQ ID No:292, NAV1.5 motif);
t. is selective for a first polypeptide comprising the sequence STMISTLEN (SEQ ID No:312, NAV1.9 motif) over a second polypeptide comprising the sequence VGTVLSDIIQ (SEQ ID No:317, NAV1.5 motif);
u. is selective for a first polypeptide comprising the sequence VSTMISTLENQEHIPFPPTLFR (SEQ ID No:52, NAV1.9 motif) over a second polypeptide comprising the sequence VGTVLSDIIQKYFFSPTLFR (SEQ ID No:142, NAV1.5 motif);
v. is selective for a first polypeptide comprising the sequence HIPFPP (SEQ ID No:297, NAV1.9 motif) over a second polypeptide comprising the sequence TYFVSP (SEQ ID No:318, NAV1.7 motif);
w. is selective for a first polypeptide comprising the sequence MISTLEN (SEQ ID No:295, NAV1.9 motif) over a second polypeptide comprising the sequence FLADLIE (SEQ ID No:293, NAV1.7 motif);
x. is selective for a first polypeptide comprising the sequence STMISTLEN (SEQ ID No:312, NAV1.9 motif) over a second polypeptide comprising the sequence GMFLADLIE (SEQ ID No:319, NAV1.7 motif);
y. is selective for a first polypeptide comprising the sequence VSTMISTLENQEHIPFPPTLFR (SEQ ID No:52, NAV1.9 motif) over a second polypeptide comprising the sequence VGMFLADLIETYFVSPTLFR (SEQ ID No:16, NAV1.7 motif);
z. is selective for a first polypeptide comprising the sequence HIPFPP (SEQ ID No:297, NAV1.9 motif) over a second polypeptide comprising the sequence QSYFSP (SEQ ID No:296, NAV1.8 motif);
aa. is selective for a first polypeptide comprising the sequence VSTMIST (SEQ ID No:294, NAV1.9 motif) over a second polypeptide comprising the sequence ASLIFSA (SEQ ID No:282, NAV1.8 motif);
bb. is selective for a first polypeptide comprising the sequence MISTLEN (SEQ ID No:295, NAV1.9 motif) over a second polypeptide comprising the sequence IFSAILK (SEQ ID No:284, NAV1.8 motif);
cc. is selective for a first polypeptide comprising the sequence STMISTLEN (SEQ ID No:312, NAV1.9 motif) over a second polypeptide comprising the sequence ASLIFSAILK (SEQ ID No:321, NAV1.8 motif);
dd. is selective for a first polypeptide comprising the sequence VSTMISTLENQEHIPFPPTLFR (SEQ ID No:52, NAV1.9 motif) over a second polypeptide comprising the sequence ASILFSAILKSLQSYFSPTLFR (SEQ ID No:34, NAV1.8 motif).

Sentence 278. An antibody or fragment according to:
a. Sentence 277a, 277b, 277c, 277d or sentence 277e, wherein the second polypeptide is a human NAV1.6 protein; or
b. Sentence 277a, 277f, 277g, 277h or sentence 277i, wherein the second polypeptide is a human NAV1.1 protein or a human NAV1.2 protein; or
c. Sentence 277a, 277f, 277j, 277k or sentence 277l, wherein the second polypeptide is a human NAV1.3 protein; or
d. Sentence 277a, 277m, 277n, 277o or sentence 277p, wherein the second polypeptide is a human NAV1.4 protein; or
e. Sentence 277q, 277r, 277s, 277t or sentence 277u, wherein the second polypeptide is a human NAV1.5 protein; or
f. Sentence 277b, 277v, 277w, 277x or sentence 277y, wherein the second polypeptide is a human NAV1.7 protein; or
g. Sentence 277z, 277aa, 277bb, 277cc or sentence 277dd, wherein the second polypeptide is a human NAV1.8 protein.

Sentence 279. An antibody or fragment according to sentence 277 or sentence 278 which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 280. An antibody or fragment according to sentence 279, wherein the antibody or fragment binds to the D4E2 loop (SEQ ID No:52) of human NAV1.9.

Sentence 281. An antibody or fragment according to any one of sentences 277 to 280, wherein the first polypeptide is a NAV1.9 protein.

Sentence 282. An antibody or fragment according to any one of sentences 277 to 281, wherein the antibody or fragment is selective for the first polypeptide over the second polypeptide by at least 10-fold, at least a 25-fold, at least 50-fold, at least 100-fold, at least 150-fold, at least 200-fold, at least 300-fold, at least 400-fold, at least 500-fold, at least 700-fold or at least 1000-fold (such as at least 200-fold, at least 300-fold, at least 400-fold, at least 500-fold, at least 700-fold or at least 1000-fold, e.g. at least at least 400-fold, at least 500-fold, at least 700-fold or at least 1000-fold), optionally as measured in a standard whole cell patch clamp assay and optionally as measured by comparison of either IC₅₀, degree of maximum inhibition or by SPR.

Sentence 283. An antibody or fragment thereof according to any one of sentences 277 to 282, which
a. has the features of sentence 277a; or
b. has one, more (e.g. 2, or 3) or all of the features of sentence 277b, sentence 277c, sentence 277d and sentence 277e; and one, more (e.g. 2, or 3) or all of the features of sentence 277m, sentence 277n, sentence 277o and sentence 277p.

Sentence 284. An antibody or fragment thereof according to any one of sentences 277 to 282, which has one, more (e.g. 2, or 3) or all of the features of sentence 277b, sentence 277c, sentence 277d and sentence 277e; and one, more (e.g. 2, 3 or 4) or all of the features of sentence 277q, sentence 277r, sentences 277s, sentence 277t and sentence 277u.

Sentence 285. An antibody or fragment thereof according to any one of sentences 277 to 282, which has one, more (e.g. 2, or 3) or all of the features of sentence 277m, sentence 277n, sentence 277o and sentence 277p; and one, more (e.g. 2, 3 or 4) or all of the features of sentence 277q, sentence 277r, sentences 277s, sentence 277t and sentence 277u.

Sentence 286. An antibody or fragment thereof according to any one of sentences 277 to 282, which has one, more (e.g. 2, or 3) or all of the features of sentence 277m, sentence 277n, sentence 277o and sentence 277p; and one, more (e.g. 2, 3 or 4) or all of the features of sentence 277q, sentence 277r, sentences 277s, sentence 277t and sentence 277u; and one, more (e.g. 2, or 3) or all of the features of sentence 277b, sentence 277c, sentence 277d and sentence 277e.

### Other Cross Reactive Antibodies

Other anti-NAV antibodies which are cross reactive with one or more other NAV proteins are described in the sentences below. Reference is made to the sentences included in the preceding section.

Sentence 100. An antibody or fragment thereof which binds to human NAV1.7 (SEQ ID No:2), and comprises one, more (e.g. 2, 3, 4 or 5), or all of the features a. to g.:
a. binds to a first polypeptide comprising the sequence LTEFV (SEQ ID No:305, NAV1.7 motif) with a comparable affinity, potency and/or efficacy to a second polypeptide comprising the sequence VGTAI (SEQ ID No:307, NAV1.8 motif);
b. binds to a first polypeptide comprising the sequence EFVNL (SEQ ID No:300, NAV1.7 motif) with a comparable affinity, potency and/or efficacy to a second polypeptide comprising the sequence TAIDL (SEQ ID No:196, NAV1.8 motif);
c. binds to a first polypeptide comprising the sequence FVNLG (SEQ ID No:173, NAV1.7 motif) with a comparable affinity, potency and/or efficacy to a second polypeptide comprising the sequence IDLRG (SEQ ID No:236, NAV1.8 motif);
d. binds to a first polypeptide comprising the sequence LTEFVNLGN (SEQ ID No:4, NAV1.7 motif) with a comparable affinity, potency and/or efficacy to a second polypeptide comprising the sequence VGTAIDLRG (SEQ ID No:22, NAV1.8 motif);
e. binds to a first polypeptide comprising the sequence LTEFV (SEQ ID No:305, NAV1.7 motif) with a comparable affinity, potency and/or efficacy to a second polypeptide comprising the sequence VSYIP (SEQ ID No:298, NAV1.9 motif);
f. binds to a first polypeptide comprising the sequence VNLGN (SEQ ID No:237, NAV1.7 motif) with a comparable affinity, potency and/or efficacy to a second polypeptide comprising the sequence GITIK (SEQ ID No:302, NAV1.9 motif); and
g. binds to a first polypeptide comprising the sequence LTEFVNLGN (SEQ ID No:4, NAV1.7 motif) with a comparable affinity, potency and/or efficacy to a second polypeptide comprising the sequence VSYIPGITIK (SEQ ID No:40, NAV1.9 motif);

Sentence 101. An antibody or fragment according to:
a. Sentence 100a, 100b, 100c or sentence 100d, wherein the second polypeptide is a human NAV1.8 protein; or
b. Sentence 100e, 100f or sentence 100g, wherein the second polypeptide is a human NAV1.9 protein.

Sentence 102. An antibody or fragment according to sentence 100 or 101, which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 103. An antibody or fragment according to sentence 102, wherein the antibody or fragment binds to the D1E2 loop (SEQ ID No:4) of human NAV1.7.

Sentence 104. An antibody or fragment according to any one of sentences 100 to 103, wherein the first polypeptide is a NAV1.7 protein.

Sentence 105. An antibody or fragment according to any one of sentences 100 to 104, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 106. An antibody or fragment according any one of sentences 100 to 104, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 117. An antibody or fragment thereof which binds to human NAV1.7 (SEQ ID No:2), and comprises one, more (e.g. 2, 3, 4 or 5), or all of the features a. to h.:
a. binds to a first polypeptide comprising the sequence VELF (SEQ ID No:176, NAV1.7 motif) with a comparable affinity, potency and/or efficacy to a second polypeptide comprising the sequence LELG (SEQ ID No:241, NAV1.8 motif);
b. binds to a first polypeptide comprising the sequence LADVEG (SEQ ID No:243, NAV1.7 motif) with a comparable affinity, potency and/or efficacy to a second polypeptide comprising the sequence VAKKGS (SEQ ID No:242, NAV1.8 motif);
c. binds to a first polypeptide comprising the sequence FLADV (SEQ ID No:259, NAV1.7 motif) with a comparable affinity, potency and/or efficacy to a second polypeptide comprising the sequence GVAKK (SEQ ID No:249, NAV1.8 motif);
d. binds to a first polypeptide comprising the sequence VELFLADVEG (SEQ ID No:8, NAV1.7 motif) with a comparable affinity, potency and/or efficacy to a second polypeptide comprising the sequence LELGVAKKGS (SEQ ID No:26, NAV1.8 motif);
e. binds to a first polypeptide comprising the sequence VELF (SEQ ID No:176, NAV1.7 motif) with a comparable affinity, potency and/or efficacy to a second polypeptide comprising the sequence ADVM (SEQ ID No:76, NAV1.9 motif);
f. binds to a first polypeptide comprising the sequence VELFLAD (SEQ ID No:180, NAV1.7 motif) with a comparable affinity, potency and/or efficacy to a second polypeptide comprising the sequence ADVMNCV (SEQ ID No:260, NAV1.9 motif);
g. binds to a first polypeptide comprising the sequence LADVEG (SEQ ID No:243, NAV1.7 motif) with a comparable affinity, potency and/or efficacy to a second polypeptide comprising the sequence VLQKRS (SEQ ID No:261, NAV1.9 motif); and
h. binds to a first polypeptide comprising the sequence VELFLADVEG (SEQ ID No:8, NAV1.7 motif) with a comparable affinity, potency and/or efficacy to a second polypeptide comprising the sequence ADVMNCVLQKRS (SEQ ID No:44, NAV1.9 motif).

Sentence 118. An antibody or fragment according to:
a. Sentence 117a, 117b, 117c or sentence 117d, wherein the second polypeptide is a human NAV1.8 protein; or
b. Sentence 117e, 117f, 117g or sentence 117h, wherein the second polypeptide is a human NAV1.9 protein.

Sentence 119. An antibody or fragment according to sentence 116 or 117, which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 120. An antibody or fragment according to sentence 119, wherein the antibody or fragment binds to the D2E2 loop (SEQ ID No:8) of human NAV1.7.

Sentence 121. An antibody or fragment according to any one of sentences 116 to 120, wherein the first polypeptide is a NAV1.7 protein.

Sentence 122. An antibody or fragment according to any one of sentences 116 to 121, wherein the affinity, potency and/or efficacy is measured by comparison of IC₅O, degree of maximum inhibition and/or SPR respectively, and optionally wherein the affinity, potency and/or efficacy are within less than 1.5-fold, at least 2-fold, at least 3-fold, at least 5-fold, at least 7-fold, at least 10-fold, at least 15-fold, at least 20-fold or at least 30-fold of each other.

Sentence 123. An antibody or fragment according any one of sentences 116 to 121, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 134. An antibody or fragment thereof which binds to human NAV1.7 (SEQ ID No:2), and comprises one, more (e.g. 2, 3, 4 or 5), or all of the features a. to l.:
a. binds to a first polypeptide comprising the sequence VTLVA (SEQ ID No:193, NAV1.7 motif) with a comparable affinity, potency and/or efficacy to a second polypeptide comprising the sequence ISLTA (SEQ ID No:202, NAV1.8 motif);
b. binds to a first polypeptide comprising the sequence VANTLG (SEQ ID No:203, NAV1.7 motif) with a comparable affinity, potency and/or efficacy to a second polypeptide comprising the sequence TAKILE (SEQ ID No:204, NAV1.8 motif);
c. binds to a first polypeptide comprising the sequence GYSDLG (SEQ ID No:205, NAV1.7 motif) with a comparable affinity, potency and/or efficacy to a second polypeptide comprising the sequence EYSEVA (SEQ ID No:206, NAV1.8 motif);
d. binds to a first polypeptide comprising the sequence TLVAN (SEQ ID No:200, NAV1.7 motif) with a comparable affinity, potency and/or efficacy to a second polypeptide comprising the sequence SLTAK (SEQ ID No:324, NAV1.8 motif);
e. binds to a first polypeptide comprising the sequence TLVANT (SEQ ID No:185, NAV1.7 motif) with a comparable affinity, potency and/or efficacy to a second polypeptide comprising the sequence SLTAKI (SEQ ID No:265, NAV1.8 motif);
f. binds to a first polypeptide comprising the sequence SDLGP (SEQ ID No:187, NAV1.7 motif) with a comparable affinity, potency and/or efficacy to a second polypeptide comprising the sequence SEVAP (SEQ ID No:264, NAV1.8 motif);
g. binds to a first polypeptide comprising the sequence TLGYSD (SEQ ID No:191, NAV1.7 motif) with a comparable affinity, potency and/or efficacy to a second polypeptide comprising the sequence ILEYSE (SEQ ID No:328, NAV1.8 motif);
h. binds to a first polypeptide comprising the sequence VTLVANTLGYSDLG (SEQ ID No:158, NAV1.7 motif) with a comparable affinity, potency and/or efficacy to a second polypeptide comprising the sequence ISLTAKILEYSEVA (SEQ ID No:162, NAV1.8 motif);
i. binds to a first polypeptide comprising the sequence VTLVANTLGYSDLGPIK (SEQ ID No:12, NAV1.7 motif) with a comparable affinity, potency and/or efficacy to a second polypeptide comprising the sequence ISLTAKILEYSEVAPIK (SEQ ID No:30, NAV1.8 motif);
j. binds to a first polypeptide comprising the sequence VTLVA (SEQ ID No:193, NAV1.7 motif) with a comparable affinity, potency and/or efficacy to a second polypeptide comprising the sequence TTLIN (SEQ ID No:207, NAV1.9 motif);
k. binds to a first polypeptide comprising the sequence LGPI (SEQ ID No:208, NAV1.7 motif) with a comparable affinity, potency and/or efficacy to a second polypeptide comprising the sequence LMEL (SEQ ID No:209, NAV1.9 motif); and
l. binds to a first polypeptide comprising the sequence VTLVANTLGYSDLGPIK (SEQ ID No:12, NAV1.7 motif) with a comparable affinity, potency and/or efficacy to a second polypeptide comprising the sequence TTLINLMELK (SEQ ID No:48, NAV1.9 motif).

Sentence 135. An antibody or fragment according to:
a. Sentence 134a, 134b, 134c, 134d, 134e, 134f, 134g, 134h or sentence 134i, wherein the second polypeptide is a human NAV1.8 protein; or
b. Sentence 134j, 134k, or sentence 134l, wherein the second polypeptide is a human NAV1.9 protein.

Sentence 136. An antibody or fragment according to sentence 134 or 135, which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 137. An antibody or fragment according to sentence 136, wherein the antibody or fragment binds to the D1E2 loop (SEQ ID No:4) of human NAV1.7.

Sentence 138. An antibody or fragment according to any one of sentences 134 to 137, wherein the first polypeptide is a NAV1.7 protein.

Sentence 139. An antibody or fragment according to any one of sentences 134 to 138, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 140. An antibody or fragment according any one of sentences 134 to 139, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 151. An antibody or fragment thereof which binds to human NAV1.7 (SEQ ID No:2), and comprises one, more (e.g. 2, 3, 4 or 5), or all of the features a. to f.:
a. binds to a first polypeptide comprising the sequence VGMFLADLIETYFV (SEQ ID No:114, NAV1.7 motif) with a comparable affinity, potency and/or efficacy to a second polypeptide comprising the sequence ASLIFSAILKSLQSYF (SEQ ID No:108, NAV1.8 motif);
b. binds to a first polypeptide comprising the sequence MFLADLIET (SEQ ID No:229, NAV1.7 motif) with a comparable affinity, potency and/or efficacy to a second polypeptide comprising the sequence LIFSAILKS (SEQ ID No:104, NAV1.8 motif);
c. binds to a first polypeptide comprising the sequence VGMFLADLIETYFVSPTLFR (SEQ ID No:16, NAV1.7 motif) with a comparable affinity, potency and/or efficacy to a second polypeptide comprising the sequence ASLIFSAILKSLQSYFSPTLFR (SEQ ID No:34, NAV1.8 motif);
d. binds to a first polypeptide comprising the sequence VGMFLADLIETYFVS (SEQ ID No:100, NAV1.7 motif) with a comparable affinity, potency and/or efficacy to a second polypeptide comprising the sequence VSTMISTLENQEHIPFP (SEQ ID No:96, NAV1.9 motif);
e. binds to a first polypeptide comprising the sequence MFLADLIET (SEQ ID No:229, NAV1.7 motif) with a comparable affinity, potency and/or efficacy to a second polypeptide comprising the sequence TMISTLEN (SEQ ID No:90, NAV1.9 motif); and
f. binds to a first polypeptide comprising the sequence VGMFLADLIETYFVSPTLFR (SEQ ID No:16), NAV1.7 motif) with a comparable affinity, potency and/or efficacy to a second polypeptide comprising the sequence STMISTLENQEHIPFPPTLFR (SEQ ID No:52, NAV1.9 motif).

Sentence 152. An antibody or fragment according to:
a. Sentence 151a, 151b or sentence 151c, wherein the second polypeptide is a human NAV1.8 protein; or
b. Sentence 151d, 151e or sentence 151f, wherein the second polypeptide is a human NAV1.9 protein.

Sentence 153. An antibody or fragment according to sentence 151 or 152, which binds to human NAV1.7 (SEQ ID No:2) with an IC₅₀ of less than 100 nM, and/or a degree of maximum inhibition of at least 50%, optionally as measured in a standard whole cell patch clamp assay.

Sentence 154. An antibody or fragment according to sentence 153, wherein the antibody or fragment binds to the D4E2 loop (SEQ ID No:16) of human NAV1.7.

Sentence 155. An antibody or fragment according to any one of sentences 151 to 154, wherein the first polypeptide is a NAV1.7 protein.

Sentence 156. An antibody or fragment according to any one of sentences 151 to 155, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 157. An antibody or fragment according any one of sentences 151 to 156, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 168. An antibody or fragment thereof which binds to human NAV1.8 (SEQ ID No:20), and comprises one, more (e.g. 2, 3, 4 or 5), or all of the features a. to f.:
a. binds to a first polypeptide comprising the sequence IDLRG (SEQ ID No:236, NAV1.8 motif) with a comparable affinity, potency and/or efficacy to a second polypeptide comprising the sequence VNLGN (SEQ ID No:172, NAV1.7 motif);
b. binds to a first polypeptide comprising the sequence VGTAIDLRG (SEQ ID No:22, NAV1.8 motif) with a comparable affinity, potency and/or efficacy to a second polypeptide comprising the sequence LTEFVNLGN (SEQ ID No:4, NAV1.7 motif);
c. binds to a first polypeptide comprising the sequence VGTAID (SEQ ID No:234, NAV1.8 motif) with a comparable affinity, potency and/or efficacy to a second polypeptide comprising the sequence LTEFVN (SEQ ID No:171, NAV1.7 motif);
d. binds to a first polypeptide comprising the sequence VGTAID (SEQ ID No:234, NAV1.8 motif) with a comparable affinity, potency and/or efficacy to a second polypeptide comprising the sequence VSYIPG (SEQ ID No:86, NAV1.9 motif);
e. binds to a first polypeptide comprising the sequence IDLRG (SEQ ID No:236, NAV1.8 motif) with a comparable affinity, potency and/or efficacy to a second polypeptide comprising the sequence GITIK (SEQ ID No:302, NAV1.9 motif); and
f. binds to a first polypeptide comprising the sequence VGTAIDLRG (SEQ ID No:22, NAV1.8 motif) with a comparable affinity, potency and/or efficacy to a second polypeptide comprising the sequence VSYIPGITIK (SEQ ID No:40, NAV1.9 motif).

Sentence 169. An antibody or fragment according to:
a. Sentence 168a, 168b or sentence 168c, wherein the second polypeptide is a human NAV1.7 protein; or
b. Sentence 168d, 168e or sentence 168f, wherein the second polypeptide is a human NAV1.9 protein.

Sentence 170. An antibody or fragment according to sentence 168 or 169, which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 171. An antibody or fragment according to sentence 170, wherein the antibody or fragment binds to the D1E2 loop (SEQ ID No:22) of human NAV1.8.

Sentence 172. An antibody or fragment according to any one of sentences 168 to 171, wherein the first polypeptide is a NAV1.8 protein.

Sentence 173. An antibody or fragment according to any one of sentences 168 to 172, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 174. An antibody or fragment according any one of sentences 168 to 172, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 185. An antibody or fragment thereof which binds to human NAV1.8 (SEQ ID No:20), and comprises one, more (e.g. 2, 3, 4 or 5), or all of the features a. to g.:
a. binds to a first polypeptide comprising the sequence VAKKGS (SEQ ID No:242, NAV1.8 motif) with a comparable affinity, potency and/or efficacy to a second polypeptide comprising the sequence LADVEG (SEQ ID No:243, NAV1.7 motif);
b. binds to a first polypeptide comprising the sequence LELG (SEQ ID No:241, NAV1.8 motif) with a comparable affinity, potency and/or efficacy to a second polypeptide comprising the sequence VELF (SEQ ID No:176, NAV1.7 motif);
c. binds to a first polypeptide comprising the sequence GVAKK (SEQ ID No:249, NAV1.8 motif) with a comparable affinity, potency and/or efficacy to a second polypeptide comprising the sequence FLADV (SEQ ID No:259, NAV1.7 motif);
d. binds to a first polypeptide comprising the sequence LELGVAKKGS (SEQ ID No:26, NAV1.8 motif) with a comparable affinity, potency and/or efficacy to a second polypeptide comprising the sequence VELFLADVEG (SEQ ID No:8, NAV1.7 motif);
e. binds to a first polypeptide comprising the sequence LELGV (SEQ ID No:246, NAV1.8 motif) with a comparable affinity, potency and/or efficacy to a second polypeptide comprising the sequence ADVMNCV (SEQ ID No:260, NAV1.9 motif);
f. binds to a first polypeptide comprising the sequence VAKKGS (SEQ ID No:242, NAV1.8 motif) with a comparable affinity, potency and/or efficacy to a second polypeptide comprising the sequence VLQKRS (SEQ ID No:261, NAV1.9 motif)
g. binds to a first polypeptide comprising the sequence LELGVAKKGS (SEQ ID No:26, NAV1.8 motif) with a comparable affinity, potency and/or efficacy to a second polypeptide comprising the sequence ADVMNCVLQKRS (SEQ ID No:44, NAV1.9 motif.

Sentence 186. An antibody or fragment according to:
a. Sentence 185a, 185b, 185c or sentence 185d, wherein the second polypeptide is a human NAV1.7 protein; or
b. Sentence 185e, 185f or sentence 185g, wherein the second polypeptide is a human NAV1.9 protein.

Sentence 187. An antibody or fragment according to sentence 185 or 186, which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 188. An antibody or fragment according to sentence 187, wherein the antibody or fragment binds to the D2E2 loop (SEQ ID No:26) of human NAV1.8.

Sentence 189. An antibody or fragment according to any one of sentences 185 to 188, wherein the first polypeptide is a NAV1.8 protein.

Sentence 190. An antibody or fragment according to any one of sentences 185 to 189, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 191. An antibody or fragment according any one of sentences 100 to 104, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 202. An antibody or fragment thereof which binds to human NAV1.8 (SEQ ID No:20), and comprises one, more (e.g. 2, 3, 4 or 5), or all of the features a. to i.:
a. binds to a first polypeptide comprising the sequence TAKILE (SEQ ID No:204, NAV1.8 motif) with a comparable affinity, potency and/or efficacy to a second polypeptide comprising the sequence VANTLG (SEQ ID No:203, NAV1.7 motif);
b. binds to a first polypeptide comprising the sequence ISLTA (SEQ ID No:202, NAV1.8 motif) with a comparable affinity, potency and/or efficacy to a second polypeptide comprising the sequence VTLVA (SEQ ID No:193, NAV1.7 motif);
c. binds to a first polypeptide comprising the sequence SEVAP (SEQ ID No:264, NAV1.8 motif) with a comparable affinity, potency and/or efficacy to a second polypeptide comprising the sequence SDLGP (SEQ ID No:187, NAV1.7 motif);
d. binds to a first polypeptide comprising the sequence SLTAKI (SEQ ID No:265, NAV1.8 motif) with a comparable affinity, potency and/or efficacy to a second polypeptide comprising the sequence TLVANT (SEQ ID No:185, NAV1.7 motif);
e. binds to a first polypeptide comprising the sequence KILEY (SEQ ID No:266, NAV1.8 motif) with a comparable affinity, potency and/or efficacy to a second polypeptide comprising the sequence ANTLGY (SEQ ID No:279, NAV1.7 motif);
f. binds to a first polypeptide comprising the sequence ISLTAKILEYSEVAPIK (SEQ ID No:30, NAV1.8 motif) with a comparable affinity, potency and/or efficacy to a second polypeptide comprising the sequence VTLVANTLGYSDLGPIK (SEQ ID No:12, NAV1.7 motif);
g. binds to a first polypeptide comprising the sequence ISLTA (SEQ ID No:202, NAV1.8 motif) with a comparable affinity, potency and/or efficacy to a second polypeptide comprising the sequence TTLIN (SEQ ID No:207, NAV1.9 motif);
h. binds to a first polypeptide comprising the sequence YSEV (SEQ ID No:280, NAV1.8 motif) with a comparable affinity, potency and/or efficacy to a second polypeptide comprising the sequence LMEL (SEQ ID No:209, NAV1.9 motif); and
i. binds to a first polypeptide comprising the sequence ISLTAKILEYSEVAPIK (SEQ ID No:30, NAV1.8 motif) with a comparable affinity, potency and/or efficacy to a second polypeptide comprising the sequence TTLINLMELK (SEQ ID No:48, NAV1.9 motif).

Sentence 203. An antibody or fragment according to:
a. Sentence 201a, 201b, 201c, 201d, 201e or sentence 201f, wherein the second polypeptide is a human NAV1.7 protein; or
b. Sentence 201g, 201h or sentence 201i, wherein the second polypeptide is a human NAV1.9 protein.

Sentence 204. An antibody or fragment according to sentence 201 or 202, which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 205. An antibody or fragment according to sentence 204, wherein the antibody or fragment binds to the D3E2 loop (SEQ ID No:30) of human NAV1.8.

Sentence 206. An antibody or fragment according to any one of sentences 201 to 205, wherein the first polypeptide is a NAV1.8 protein.

Sentence 207. An antibody or fragment according to any one of sentences 201 to 206, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 208. An antibody or fragment according any one of sentences 201 to 206, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 219. An antibody or fragment thereof which binds to human NAV1.8 (SEQ ID No:20), and comprises one, more (e.g. 2, 3, 4 or 5), or all of the features a. to h.:
a. binds to a first polypeptide comprising the sequence ASLIFSA (SEQ ID No:282, NAV1.8 motif) with a comparable affinity, potency and/or efficacy to a second polypeptide comprising the sequence VGMFLAD (SEQ ID No:283, NAV1.7 motif);
b. binds to a first polypeptide comprising the sequence IFSAILK (SEQ ID No:284, NAV1.8 motif) with a comparable affinity, potency and/or efficacy to a second polypeptide comprising the sequence FLADLIE (SEQ ID No:293, NAV1.7 motif);
c. binds to a first polypeptide comprising the sequence QSYFSP (SEQ ID No:296, NAV1.8 motif) with a comparable affinity, potency and/or efficacy to a second polypeptide comprising the sequence TYFVSP (SEQ ID No:318, NAV1.7 motif);
d. binds to a first polypeptide comprising the sequence ASLIFSAILKSLQSYFSPTLFR (SEQ ID No:34, NAV1.8 motif) with a comparable affinity, potency and/or efficacy to a second polypeptide comprising the sequence VGMFLADLIETYFVSPTLFR (SEQ ID No:16, NAV1.7 motif);
e. binds to a first polypeptide comprising the sequence ASLIFSA (SEQ ID No:282, NAV1.8 motif) with a comparable affinity, potency and/or efficacy to a second polypeptide comprising the sequence VSTMIST (SEQ ID No:294, NAV1.9 motif);
f. binds to a first polypeptide comprising the sequence IFSAILK (SEQ ID No:284, NAV1.8 motif) with a comparable affinity, potency and/or efficacy to a second polypeptide comprising the sequence MISTLEN (SEQ ID No:295, NAV1.9 motif);
g. binds to a first polypeptide comprising the sequence QSYFSP (SEQ ID No:296, NAV1.8 motif) with a comparable affinity, potency and/or efficacy to a second polypeptide comprising the sequence HIPFPP (SEQ ID No:297, NAV1.9 motif); and
h. binds to a first polypeptide comprising the sequence ASLIFSAILKSLQSYFSPTLFR (SEQ ID No:34, NAV1.8 motif) with a comparable affinity, potency and/or efficacy to a second polypeptide comprising the sequence VSTMISTLENQEHIPFPPTLFR (SEQ ID No:52, NAV1.9 motif).

Sentence 220. An antibody or fragment according to:
a. Sentence 219a, 219b, 219c or sentence 219d, wherein the second polypeptide is a human NAV1.7 protein; or
b. Sentence 219e, 219f, 219g or sentence 219h, wherein the second polypeptide is a human NAV1.9 protein.

Sentence 221. An antibody or fragment according to sentence 219 or 220, which binds to human NAV1.8 (SEQ ID No:20) with an IC₅₀ of less than 100 nM, and/or a degree of maximum inhibition of at least 50%, optionally as measured in a standard whole cell patch clamp assay.

Sentence 222. An antibody or fragment according to sentence 221, wherein the antibody or fragment binds to the D4E2 loop (SEQ ID No:34) of human NAV1.8.

Sentence 223. An antibody or fragment according to any one of sentences 219 to 222, wherein the first polypeptide is a NAV1.8 protein.

Sentence 224. An antibody or fragment according to any one of sentences 219 to 223, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 225. An antibody or fragment according any one of sentences 219 to 223, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 236. An antibody or fragment thereof which specifically binds to human NAV1.9 (SEQ ID No:38), and comprises one, more (e.g. 2, 3, 4 or 5), or all of the features a. to h.:
a. binds to a first polypeptide comprising the sequence YIPGI (SEQ ID No:301, NAV1.9 motif) with a comparable affinity, potency and/or efficacy to a second polypeptide comprising the sequence EFVNL (SEQ ID No:300, NAV1.7 motif);
b. binds to a first polypeptide comprising the sequence GITIK (SEQ ID No:302, NAV1.9 motif) with a comparable affinity, potency and/or efficacy to a second polypeptide comprising the sequence VNLGN (SEQ ID No:237, NAV1.7 motif);
c. binds to a first polypeptide comprising the sequence VSYIP (SEQ ID No:298, NAV1.9 motif) with a comparable affinity, potency and/or efficacy to a second polypeptide comprising the sequence LTEFV (SEQ ID No:305, NAV1.7 motif);
d. binds to a first polypeptide comprising the sequence VSYIPGITIK (SEQ ID No:40, NAV1.9 motif) with a comparable affinity, potency and/or efficacy to a second polypeptide comprising the sequence LTEFVNLGN (SEQ ID No:4, NAV1.7 motif);
e. binds to a first polypeptide comprising the sequence VSYIP (SEQ ID No:298, NAV1.9 motif) with a comparable affinity, potency and/or efficacy to a second polypeptide comprising the sequence VGTAI (SEQ ID No:307, NAV1.8 motif);
f. binds to a first polypeptide comprising the sequence YIPGI (SEQ ID No:301, NAV1.9 motif) with a comparable affinity, potency and/or efficacy to a second polypeptide comprising the sequence TAIDL (SEQ ID No:196, NAV1.8 motif);
g. binds to a first polypeptide comprising the sequence GITIK (SEQ ID No:302, NAV1.9 motif) with a comparable affinity, potency and/or efficacy to a second polypeptide comprising the sequence IDLRG (SEQ ID No:236, NAV1.8 motif); and
h. binds to a first polypeptide comprising the sequence VSYIPGITIK (SEQ ID No:40, NAV1.9 motif) with a comparable affinity, potency and/or efficacy to a second polypeptide comprising the sequence VGTAIDLRG (SEQ ID No:22, NAV1.8 motif).

Sentence 237. An antibody or fragment according to:
a. Sentence 236a, 236b, 236c or sentence 236d, wherein the second polypeptide is a human NAV1.7 protein; or
b. Sentence 236e, 236f, 236g or sentence 236h, wherein the second polypeptide is a human NAV1.8 protein.

Sentence 238. An antibody or fragment according to sentence 236 or 237, which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 239. An antibody or fragment according to sentence 238, wherein the antibody or fragment binds to the D1E2 loop (SEQ ID No:40) of human NAV1.9.

Sentence 240. An antibody or fragment according to any one of sentences 236 to 239, wherein the first polypeptide is a NAV1.8 protein.

Sentence 241. An antibody or fragment according to any one of sentences 236 to 240, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 242. An antibody or fragment according any one of sentences 236 to 124o, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 253. An antibody or fragment thereof which specifically binds to human NAV1.9 (SEQ ID No:38), and comprises one, more (e.g. 2, 3, 4 or 5), or all of the features a. to f.:
a. binds to a first polypeptide comprising the sequence ADVM (SEQ ID No:76, NAV1.9 motif) with a comparable affinity, potency and/or efficacy to a second polypeptide comprising the sequence VELF (SEQ ID No:176, NAV1.7 motif);
b. binds to a first polypeptide comprising the sequence VLQKRS (SEQ ID No:261, NAV1.9 motif) with a comparable affinity, potency and/or efficacy to a second polypeptide comprising the sequence LADVEG (SEQ ID No:243, NAV1.7 motif);
c. binds to a first polypeptide comprising the sequence ADVMNCVLQKRS (SEQ ID No:44, NAV1.9 motif) with a comparable affinity, potency and/or efficacy to a second polypeptide comprising the sequence VELFLANVEG (SEQ ID No:8, NAV1.7 motif);
d. binds to a first polypeptide comprising the sequence ADVM (SEQ ID No:76, NAV1.9 motif) with a comparable affinity, potency and/or efficacy to a second polypeptide comprising the sequence LELG (SEQ ID No:241, NAV1.8 motif);
e. binds to a first polypeptide comprising the sequence VLQKRS (SEQ ID No:261, NAV1.9 motif) with a comparable affinity, potency and/or efficacy to a second polypeptide comprising the sequence VAKKGS (SEQ ID No:242, NAV1.8 motif); and
f. binds to a first polypeptide comprising the sequence ADVMNCVLQKRS (SEQ ID No:44, NAV1.9 motif) with a comparable affinity, potency and/or efficacy to a second polypeptide comprising the sequence LELGVAKKGS (SEQ ID No:26, NAV1.8 motif).

Sentence 254. An antibody or fragment according to:
a. Sentence 253a, 253b or sentence 253c, wherein the second polypeptide is a human NAV1.7 protein; or
b. Sentence 253d, 253e or sentence 253f, wherein the second polypeptide is a human NAV1.8 protein.

Sentence 255. An antibody or fragment according to sentence 253 or 254, which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 256. An antibody or fragment according to sentence 255, wherein the antibody or fragment binds to the D2E2 loop (SEQ ID No:44) of human NAV1.9.

Sentence 257. An antibody or fragment according to any one of sentences 253 to 256, wherein the first polypeptide is a NAV1.8 protein.

Sentence 258. An antibody or fragment according to any one of sentences 253 to 257, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 259. An antibody or fragment according any one of sentences 253 to 257, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 270. An antibody or fragment thereof which binds to human NAV1.9 (SEQ ID No:38), and comprises one, more (e.g. 2, 3, 4 or 5), or all of the features a. to f.:
a. binds to a first polypeptide comprising the sequence LMELK (SEQ ID No:275, NAV1.9 motif) with a comparable affinity, potency and/or efficacy to a second polypeptide comprising the sequence LGPIK (SEQ ID No:308, NAV1.7 motif);
b. binds to a first polypeptide comprising the sequence TTLIN (SEQ ID No:207, NAV1.9 motif) with a comparable affinity, potency and/or efficacy to a second polypeptide comprising the sequence VTLVA (SEQ ID No:193, NAV1.7 motif);
c. binds to a first polypeptide comprising the sequence TTLINLMELK (SEQ ID No:48, NAV1.9 motif) with a comparable affinity, potency and/or efficacy to a second polypeptide comprising the sequence VTLVANTLGYSDLGPIK (SEQ ID No:12, NAV1.7 motif);
d. binds to a first polypeptide comprising the sequence TTLIN (SEQ ID No:207, NAV1.9 motif) with a comparable affinity, potency and/or efficacy to a second polypeptide comprising the sequence ISLTA (SEQ ID No:202, NAV1.8 motif);
e. binds to a first polypeptide comprising the sequence LMELK (SEQ ID No:275, NAV1.9 motif) with a comparable affinity, potency and/or efficacy to a second polypeptide comprising the sequence VAPIK (SEQ ID No:310, NAV1.8 motif); and
f. binds to a first polypeptide comprising the sequence TTLINLMELK (SEQ ID No:48, NAV1.9 motif) with a comparable affinity, potency and/or efficacy to a second polypeptide comprising the sequence ISLTAKILEYSEVAPIK (SEQ ID No:30, NAV1.8 motif).

Sentence 271. An antibody or fragment according to:
a. Sentence 270a, 270b, or sentence 270c, wherein the second polypeptide is a human NAV1.7 protein; or
b. Sentence 270d, 270e or sentence 270f, wherein the second polypeptide is a human NAV1.8 protein.

Sentence 272. An antibody or fragment according to sentence 270 or 271, which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 273. An antibody or fragment according to sentence 272, wherein the antibody or fragment binds to the D3E2 loop (SEQ ID No:48) of human NAV1.9.

Sentence 274. An antibody or fragment according to any one of sentences 270 to 273, wherein the first polypeptide is a NAV1.8 protein.

Sentence 275. An antibody or fragment according to any one of sentences 270 to 274, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 276. An antibody or fragment according any one of sentences 270 to 274, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

Sentence 287. An antibody or fragment thereof which binds to human NAV1.9 (SEQ ID No:38), and comprises one, more (e.g. 2, 3, 4 or 5), or all of the features a. to j.:
a. binds to a first polypeptide comprising the sequence VSTMIST (SEQ ID No:294, NAV1.9 motif) with a comparable affinity, potency and/or efficacy to a second polypeptide comprising the sequence VGMFLAD (SEQ ID No:283, NAV1.7 motif);
b. binds to a first polypeptide comprising the sequence HIPFPP (SEQ ID No:297, NAV1.9 motif) with a comparable affinity, potency and/or efficacy to a second polypeptide comprising the sequence TYFVSP (SEQ ID No:318, NAV1.7 motif);
c. binds to a first polypeptide comprising the sequence MISTLEN (SEQ ID No:295, NAV1.9 motif) with a comparable affinity, potency and/or efficacy to a second polypeptide comprising the sequence FLADLIE (SEQ ID No:293, NAV1.7 motif);
d. binds to a first polypeptide comprising the sequence STMISTLEN (SEQ ID No:312, NAV1.9 motif) with a comparable affinity, potency and/or efficacy to a second polypeptide comprising the sequence GMFLADLIE (SEQ ID No:319, NAV1.7 motif);
e. binds to a first polypeptide comprising the sequence VSTMISTLENQEHIPFPPTLFR (SEQ ID No:52, NAV1.9 motif) with a comparable affinity, potency and/or efficacy to a second polypeptide comprising the sequence VGMFLADLIETYFVSPTLFR (SEQ ID No:16, NAV1.7 motif);
f. binds to a first polypeptide comprising the sequence HIPFPP (SEQ ID No:297, NAV1.9 motif) with a comparable affinity, potency and/or efficacy to a second polypeptide comprising the sequence QSYFSP (SEQ ID No:296, NAV1.8 motif);
g. binds to a first polypeptide comprising the sequence VSTMIST (SEQ ID No:294, NAV1.9 motif) with a comparable affinity, potency and/or efficacy to a second polypeptide comprising the sequence ASLIFSA (SEQ ID No:282, NAV1.8 motif);
h. binds to a first polypeptide comprising the sequence MISTLEN (SEQ ID No:295, NAV1.9 motif) with a comparable affinity, potency and/or efficacy to a second polypeptide comprising the sequence IFSAILK (SEQ ID No:284, NAV1.8 motif);
i. binds to a first polypeptide comprising the sequence STMISTLEN (SEQ ID No:312, NAV1.9 motif) with a comparable affinity, potency and/or efficacy to a second polypeptide comprising the sequence ASLIFSAILK (SEQ ID No:321, NAV1.8 motif); and
j. binds to a first polypeptide comprising the sequence VSTMISTLENQEHIPFPPTLFR (SEQ ID No:52, NAV1.9 motif) with a comparable affinity, potency and/or efficacy to a second polypeptide comprising the sequence ASILFSAILKSLQSYFSPTLFR (SEQ ID No:34, NAV1.8 motif).

Sentence 288. An antibody or fragment according to:
a. Sentence 288a, 288b, 288c, 288d or sentence 288e, wherein the second polypeptide is a human NAV1.7 protein; or
b. Sentence 288f, 288g, 288h, 288i or sentence 288j, wherein the second polypeptide is a human NAV1.8 protein.

Sentence 289. An antibody or fragment according to sentence 288 or 289, which binds to human NAV1.9 (SEQ ID No:38) with an IC₅₀ of less than 100 nM, and/or a degree of maximum inhibition of at least 50%, optionally as measured in a standard whole cell patch clamp assay.

Sentence 290. An antibody or fragment according to sentence 289, wherein the antibody or fragment binds to the D4E2 loop (SEQ ID No:52) of human NAV1.9.

Sentence 291. An antibody or fragment according to any one of sentences 288 to 290, wherein the first polypeptide is a NAV1.8 protein.

Sentence 292. An antibody or fragment according to any one of sentences 288 to 291, wherein the affinity, potency and/or efficacy is measured by comparison of IC₅₀, degree of maximum inhibition and/or SPR respectively, and optionally wherein the affinity, potency and/or efficacy are within at least 1.5-fold, at least 2-fold, at least 3-fold, at least 5-fold, at least 7-fold, at least 10-fold, at least 15-fold, at least 20-fold or at least 30-fold of each other.

Sentence 293. An antibody or fragment according any one of sentences 288 to 291, wherein the potency is measured by IC₅₀, and the IC₅₀ is less than 10 nM, less than 7 nM, less than 5 nM, less than 3 nM or less than 1 nM for both the first polypeptide and for the second polypeptide; or wherein the efficacy is measured by degree of maximum inhibition, and the degree of maximum inhibition is greater than 50%, greater than 60%, greater than 70%, greater than 80%, greater than 90% or greater than 95% for both the first polypeptide and for the second polypeptide.

The priority application, filed as GB1418713.2 on 21 Oct. 2014 and published with this PCT application, contains 398 claims, which can be found on pages 203 to 298 which are specifically incorporated herein by reference.

Further embodiments of the invention may also be described according to the following aspects set out below. For the avoidance of doubt, all other embodiments, aspects and configurations of the invention disclosed herein may equally be applied to these sentences as appropriate.

Sentence 1. An antibody or fragment thereof which binds to human Nav1.7 (Seq ID No:2) and comprises a VH region which comprises:
a. a CDRH3 sequence of 22D04 selected from SEQ ID Nos: 338 and 339;
b. a CDRH3 sequence of 22G08 selected from SEQ ID Nos: 354 and 355;
c. a CDRH3 sequence of 22G09 selected from SEQ ID Nos: 370 and 371;
d. a CDRH3 sequence of 25A01 selected from SEQ ID Nos: 386 and 387;
e. a CDRH3 sequence of 25C01 selected from SEQ ID Nos: 402 and 403;
f. a CDRH3 sequence of 25F08 selected from SEQ ID Nos: 418 and 419;
g. a CDRH3 sequence of 28B08 selected from SEQ ID Nos: 450 and 451;
h. a CDRH3 sequence of 28C11 selected from SEQ ID Nos: 466 and 467;
i. a CDRH3 sequence of 32B04 selected from SEQ ID Nos: 514 and 515;
j. a CDRH3 sequence of 32D04 selected from SEQ ID Nos: 530 and 531;
k. a CDRH3 sequence of 35A06 selected from SEQ ID Nos: 562 and 563; or
l. a CDRH3 sequence of 35E11 selected from SEQ ID Nos: 594 and 595.

Sentence 2. An antibody or fragment according to sentence 1, wherein:
a. for part a), the antibody or fragment further comprises a CDRH1 sequence of 22D04 selected from SEQ ID Nos: 334 and 335;
b. for part b), the antibody or fragment further comprises a CDRH1 sequence of 22G08 selected from SEQ ID Nos: 350 and 351;
c. for part c), the antibody or fragment further comprises a CDRH1 sequence of 22G09 selected from SEQ ID Nos: 366 and 367;
d. for part d), the antibody or fragment further comprises a CDRH1 sequence of 25A01 selected from SEQ ID Nos: 382 and 383;
e. for part e), the antibody or fragment further comprises a CDRH1 sequence of 25C01 selected from SEQ ID Nos: 398 and 399;
f. for part f), the antibody or fragment further comprises a CDRH1 sequence of 25F08 selected from SEQ ID Nos: 414 and 415;
g. for part g), the antibody or fragment further comprises a CDRH1 sequence of 28B08 selected from SEQ ID Nos: 446 and 447;
h. for part h), the antibody or fragment further comprises a CDRH1 sequence of 28C11 selected from SEQ ID Nos: 462 and 463;
i. for part i), the antibody or fragment further comprises a CDRH1 sequence of 32B04 selected from SEQ ID Nos: 510 and 511;
j. for part j), the antibody or fragment further comprises a CDRH1 sequence of 32D04 selected from SEQ ID Nos: 526 and 527;
k. for part k), the antibody or fragment further comprises a CDRH1 sequence of 35A06 selected from SEQ ID Nos: 558 and 559; or
l. for part l), the antibody or fragment further comprises a CDRH1 sequence of 35E11 selected from SEQ ID Nos: 590 and 591.

Sentence 3. An antibody or fragment according to sentence 1 or sentence 2, wherein:
a. for part a), the antibody or fragment further comprises a CDRH2 sequence of 22D04 selected from SEQ ID Nos: 336 and 337;
b. for part b), the antibody or fragment further comprises a CDRH2 sequence of 22G08 selected from SEQ ID Nos: 352 and 353;
c. for part c), the antibody or fragment further comprises a CDRH2 sequence of 22G09 selected from SEQ ID Nos: 368 and 369;
d. for part d), the antibody or fragment further comprises a CDRH2 sequence of 25A01 selected from SEQ ID Nos: 384 and 385;
e. for part e), the antibody or fragment further comprises a CDRH2 sequence of 25C01 selected from SEQ ID Nos: 400 and 401;
f. for part f), the antibody or fragment further comprises a CDRH2 sequence of 25F08 selected from SEQ ID Nos: 416 and 417;
g. for part g), the antibody or fragment further comprises a CDRH2 sequence of 28B08 selected from SEQ ID Nos: 448 and 449;
h. for part h), the antibody or fragment further comprises a CDRH2 sequence of 28C11 selected from SEQ ID Nos: 464 and 465;
i. for part i), the antibody or fragment further comprises a CDRH2 sequence of 32B04 selected from SEQ ID Nos: 512 and 513;
j. for part j), the antibody or fragment further comprises a CDRH2 sequence of 32D04 selected from SEQ ID Nos: 528 and 529;
k. for part k), the antibody or fragment further comprises a CDRH2 sequence of 35A06 selected from SEQ ID Nos: 560 and 561; or
l. for part l), the antibody or fragment further comprises a CDRH2 sequence of 35E11 selected from SEQ ID Nos: 592 and 593.

Sentence 4. An antibody or fragment according to any one of sentences 1 to 3, wherein:
a. for part a), the antibody or fragment comprises the VH region of 22D04 (SEQ ID No: 331);
b. for part b), the antibody or fragment comprises the VH region of 22G08 (SEQ ID No: 347);
c. for part c), the antibody or fragment comprises the VH region of 22G09 (SEQ ID No: 363);
d. for part d), the antibody or fragment comprises the VH region of 25A01 (SEQ ID No: 379);
e. for part e), the antibody or fragment comprises the VH region of 25C01 (SEQ ID No: 395);
f. for part f), the antibody or fragment comprises the VH region of 25F08 (SEQ ID No: 411);
g. for part g), the antibody or fragment comprises the VH region of 28B08 (SEQ ID No: 443);
h. for part h), the antibody or fragment comprises the VH region of 28C11 (SEQ ID No: 459);
i. for part i), the antibody or fragment comprises the VH region of 32B04 (SEQ ID No: 507);
j. for part j), the antibody or fragment comprises the VH region of 32D04 (SEQ ID No: 523);
k. for part k), the antibody or fragment comprises the VH region of 35A06 (SEQ ID No: 555); or
l. for part l), the antibody or fragment comprises the VH region of 35E11 (SEQ ID No: 588).

Sentence 5. An antibody or fragment according to any one of sentences 1 to 4, which further comprises a V_{L} region which comprises:
a. for part a), a CDRL1 sequence of 22D04 selected from SEQ ID Nos: 340 and 341;
b. for part b), a CDRL1 sequence of 22G08 selected from SEQ ID Nos: 356 and 357;
c. for part c), a CDRL1 sequence of 22G09 selected from SEQ ID Nos: 372 and 373;
d. for part d), a CDRL1 sequence of 25A01 selected from SEQ ID Nos: 388 and 389;
e. for part e), a CDRL1 sequence of 25C01 selected from SEQ ID Nos: 404 and 405;
f. for part f), a CDRL1 sequence of 25F08 selected from SEQ ID Nos: 420 and 421;
g. for part g), a CDRL1 sequence of 28B08 selected from SEQ ID Nos: 452 and 453;
h. for part h), a CDRL1 sequence of 28C11 selected from SEQ ID Nos: 468 and 469;
i. for part i), a CDRL1 sequence of 32B04 selected from SEQ ID Nos: 516 and 517;
j. for part j), a CDRL1 sequence of 32D04 selected from SEQ ID Nos: 532 and 533;
k. for part k), a CDRL1 sequence of 35A06 selected from SEQ ID Nos: 564 and 565; or
l. for part l), a CDRL1 sequence of 35E11 selected from SEQ ID Nos: 596 and 597.

Sentence 6. An antibody or fragment according to any one of sentences 1 to 5, which further comprises a VL region which comprises:
a. for part a), a CDRL2 sequence of 22D04 selected from SEQ ID Nos: 342 and 343;
b. for part b), a CDRL2 sequence of 22G08 selected from SEQ ID Nos: 358 and 359;
c. for part c), a CDRL2 sequence of 22G09 selected from SEQ ID Nos: 374 and 375;
d. for part d), a CDRL2 sequence of 25A01 selected from SEQ ID Nos: 390 and 391;
e. for part e), a CDRL2 sequence of 25C01 selected from SEQ ID Nos: 406 and 407;
f. for part f), a CDRL2 sequence of 25F08 selected from SEQ ID Nos: 422 and 423;
g. for part g), a CDRL2 sequence of 28B08 selected from SEQ ID Nos: 454 and 455;
h. for part h), a CDRL2 sequence of 28C11 selected from SEQ ID Nos: 470 and 471;
i. for part i), a CDRL2 sequence of 32B04 selected from SEQ ID Nos: 518 and 519;
j. for part j), a CDRL2 sequence of 32D04 selected from SEQ ID Nos: 534 and 535;
k. for part k), a CDRL2 sequence of 35A06 selected from SEQ ID Nos: 566 and 567; or
l. for part l), a CDRL2 sequence of 35E11 selected from SEQ ID Nos: 598 and 599.

Sentence 7. An antibody or fragment according to any one of sentences 1 to 6, which further comprises a VL region which comprises:
a. for part a), a CDRL3 sequence of 22D04 selected from SEQ ID Nos: 344 and 345;
b. for part b), a CDRL3 sequence of 22G08 selected from SEQ ID Nos: 360 and 361;
c. for part c), a CDRL3 sequence of 22G09 selected from SEQ ID Nos: 376 and 377;
d. for part d), a CDRL3 sequence of 25A01 selected from SEQ ID Nos: 392 and 393;
e. for part e), a CDRL3 sequence of 25C01 selected from SEQ ID Nos: 408 and 409;
f. for part f), a CDRL3 sequence of 25F08 selected from SEQ ID Nos: 424 and 425;
g. for part g), a CDRL3 sequence of 28B08 selected from SEQ ID Nos: 456 and 457;
h. for part h), a CDRL3 sequence of 28C11 selected from SEQ ID Nos: 472 and 473;
i. for part i), a CDRL3 sequence of 32B04 selected from SEQ ID Nos: 520 and 521;
j. for part j), a CDRL3 sequence of 32D04 selected from SEQ ID Nos: 536 and 537;
k. for part k), a CDRL3 sequence of 35A06 selected from SEQ ID Nos: 568 and 569; or
l. for part l), a CDRL3 sequence of 35E11 selected from SEQ ID Nos: 600 and 601.

Sentence 8. An antibody or fragment according to any one of sentences 1 to 7, wherein:
a. for part a), the antibody or fragment comprises the VL region of 22D04 (SEQ ID No: 333);
b. for part b), the antibody or fragment comprises the VL region of 22G08 (SEQ ID No: 349);
c. for part c), the antibody or fragment comprises the VL region of 22G09 (SEQ ID No: 365);
d. for part d), the antibody or fragment comprises the VL region of 25A01 (SEQ ID No: 381);
e. for part e), the antibody or fragment comprises the VL region of 25C01 (SEQ ID No: 397);
f. for part f), the antibody or fragment comprises the VL region of 25F08 (SEQ ID No: 413);
g. for part g), the antibody or fragment comprises the VL region of 28B08 (SEQ ID No: 445);
h. for part h), the antibody or fragment comprises the VL region of 28C11 (SEQ ID No: 461);
i. for part i), the antibody or fragment comprises the VL region of 32B04 (SEQ ID No: 509);
j. for part j), the antibody or fragment comprises the VL region of 32D04 (SEQ ID No: 525);
k. for part k), the antibody or fragment comprises the VL region of 35A06 (SEQ ID No: 557); or
l. for part l), the antibody or fragment comprises the VL region of 35E11 (SEQ ID No: 589).

The antibodies and fragments described in sentences 1 to 8 may comprise 1, 2, 3, 4, 5 or all 6 CDR regions. Therefore, the following discussion regarding antibodies equally encompasses fragments of antibodies as appropriate.

In one aspect, there is provided an antibody which has the CDRH1 and CDRH2 sequences of 22D04. In another aspect, there is provided an antibody which has the CDRH1 and CDRH3 sequences of 22D04. In another aspect, there is provided an antibody which has the CDRH1 and CDRL1 sequences of 22D04. In another aspect, there is provided an antibody which has the CDRH1 and CDRL2 sequences of 22D04. In another aspect, there is provided an antibody which has the CDRH1 and CDRL3 sequences of 22D04. In another aspect, there is provided an antibody which has the CDRH2 and CDRH3 sequences of 22D04. In another aspect, there is provided an antibody which has the CDRH2 and CDRL1 sequences of 22D04. In another aspect, there is provided an antibody which has the CDRH2 and CDRL2 sequences of 22D04. In another aspect, there is provided an antibody which has the CDRH2 and CDRL3 sequences of 22D04. In another aspect, there is provided an antibody which has the CDRH3 and CDRL1 sequences of 22D04. In another aspect, there is provided an antibody which has the CDRH3 and CDRL2 sequences of 22D04. In another aspect, there is provided an antibody which has the CDRH3 and CDRL3 sequences of 22D04. In another aspect, there is provided an antibody which has the CDRL1 and CDRL2 sequences of 22D04. In another aspect, there is provided an antibody which has the CDRL1 and CDRL3 sequences of 22D04. In another aspect, there is provided an antibody which has the CDRL2 and CDRL3 sequences of 22D04. In another aspect, there is provided an antibody which has the CDRH1, CDRH2 and CDRH3 sequences of 22D04. In another aspect, there is provided an antibody which has the CDRH1, CDRH2 and CDRL1 sequences of 22D04. In another aspect, there is provided an antibody which has the CDRH1, CDRH2 and CDRL2 sequences of 22D04. In another aspect, there is provided an antibody which has the CDRH1, CDRH2 and CDRL3 sequences of 22D04. In another aspect, there is provided an antibody which has the CDRH1, CDRH3 and CDRL1 sequences of 22D04. In another aspect, there is provided an antibody which has the CDRH1, CDRH3 and CDRL2 sequences of 22D04. In another aspect, there is provided an antibody which has the CDRH1, CDRH3 and CDRL3 sequences of 22D04. In another aspect, there is provided an antibody which has the CDRH1, CDRL1 and CDRL2 sequences of 22D04. In another aspect, there is provided an antibody which has the CDRH1, CDRL1 and CDRL3 sequences of 22D04. In another aspect, there is provided an antibody which has the CDRH1, CDRL2 and CDRL3 sequences of 22D04. In another aspect, there is provided an antibody which has the CDRH2, CDRH3 and CDRL1 sequences of 22D04. In another aspect, there is provided an antibody which has the CDRH2, CDRL1 and CDRL2 sequences of 22D04. In another aspect, there is provided an antibody which has the CDRH2, CDRL1 and CDRL3 sequences of 22D04. In another aspect, there is provided an antibody which has the CDRH3, CDRL1 and CDRL2 sequences of 22D04. In another aspect, there is provided an antibody which has the CDRH3, CDRL1 and CDRL3 sequences of 22D04. In another aspect, there is provided an antibody which has the CDRH3, CDRL2 and CDRL3 sequences of 22D04. In another aspect, there is provided an antibody which has the CDRL1, CDRL2 and CDRL3 sequences of 22D04.

In another aspect, there is provided an antibody which has the CDRH1, CDRH2, CDRH3 and CDRL1 sequences of 22D04. In another aspect, there is provided an antibody which has the CDRH1, CDRH2, CDRH3 and CDRL2 sequences of 22D04. In another aspect, there is provided an antibody which has the CDRH1, CDRH2, CDRH3 and CDRL3 sequences of 22D04. In another aspect, there is provided an antibody which has the CDRH1, CDRH3, CDRL1 and CDRL2 sequences of 22D04. In another aspect, there is provided an antibody which has the CDRH1, CDRH3, CDRL1 and CDRL3 sequences of 22D04. In another aspect, there is provided an antibody which has the CDRH1, CDRH3, CDRL1 and CDRL2 sequences of 22D04. In another aspect, there is provided an antibody which has the CDRH1, CDRH3, CDRL1 and CDRL3 sequences of 22D04. In another aspect, there is provided an antibody which has the CDRH1, CDRL1, CDRL2 and CDRL3 sequences of 22D04. In another aspect, there is provided an antibody which has the CDRH2, CDRH3, CDRL1 and CDRL2 sequences of 22D04. In another aspect, there is provided an antibody which has the CDRH2, CDRH3, CDRL1 and CDRL3 sequences of 22D04. In another aspect, there is provided an antibody which has the CDRH2, CDRL1, CDRL2 and CDRL3 sequences of 22D04. In another aspect, there is provided an antibody which has the CDRH3, CDRL1, CDRL2 and CDRL3 sequences of 22D04.

In another aspect, there is provided an antibody which has the CDRH1, CDRH2, CDRH3, CDRL1 and CDRL2 sequences of 22D04. In another aspect, there is provided an antibody which has the CDRH1, CDRH2, CDRH3, CDRL1 and CDRL3 sequences of 22D04. In another aspect, there is provided an antibody which has the CDRH1, CDRH3, CDRL1, CDRL2 and CDRL3 sequences of 22D04. In another aspect, there is provided an antibody which has the CDRH1, CDRH2, CDRL1, CDRL2 and CDRL3 sequences of 22D04. In another aspect, there is provided an antibody which has the CDRH2, CDRH3, CDRL1, CDRL2 and CDRL3 sequences of 22D04. In another aspect, there is provided an antibody which has the CDRH1, CDRH2, CDRH3, CDRL2 and CDRL3 sequences of 22D04.

In another aspect, there is provided an antibody which has the CDRH1, CDRH2, CDRH3, CDRL1, CDRL2 and CDRL3 sequences of 22D04.

In one aspect, there is provided an antibody which has the CDRH1 and CDRH2 sequences of 22G08. In another aspect, there is provided an antibody which has the CDRH1 and CDRH3 sequences of 22G08. In another aspect, there is provided an antibody which has the CDRH1 and CDRL1 sequences of 22G08. In another aspect, there is provided an antibody which has the CDRH1 and CDRL2 sequences of 22G08. In another aspect, there is provided an antibody which has the CDRH1 and CDRL3 sequences of 22G08. In another aspect, there is provided an antibody which has the CDRH2 and CDRH3 sequences of 22G08. In another aspect, there is provided an antibody which has the CDRH2 and CDRL1 sequences of 22G08. In another aspect, there is provided an antibody which has the CDRH2 and CDRL2 sequences of 22G08. In another aspect, there is provided an antibody which has the CDRH2 and CDRL3 sequences of 22G08. In another aspect, there is provided an antibody which has the CDRH3 and CDRL1 sequences of 22G08. In another aspect, there is provided an antibody which has the CDRH3 and CDRL2 sequences of 22G08. In another aspect, there is provided an antibody which has the CDRH3 and CDRL3 sequences of 22G08. In another aspect, there is provided an antibody which has the CDRL1 and CDRL2 sequences of 22G08. In another aspect, there is provided an antibody which has the CDRL1 and CDRL3 sequences of 22G08. In another aspect, there is provided an antibody which has the CDRL2 and CDRL3 sequences of 22G08. In another aspect, there is provided an antibody which has the CDRH1, CDRH2 and CDRH3 sequences of 22G08. In another aspect, there is provided an antibody which has the CDRH1, CDRH2 and CDRL1 sequences of 22G08. In another aspect, there is provided an antibody which has the CDRH1, CDRH2 and CDRL2 sequences of 22G08. In another aspect, there is provided an antibody which has the CDRH1, CDRH2 and CDRL3 sequences of 22G08. In another aspect, there is provided an antibody which has the CDRH1, CDRH3 and CDRL1 sequences of 22G08. In another aspect, there is provided an antibody which has the CDRH1, CDRH3 and CDRL2 sequences of 22G08. In another aspect, there is provided an antibody which has the CDRH1, CDRH3 and CDRL3 sequences of 22G08. In another aspect, there is provided an antibody which has the CDRH1, CDRL1 and CDRL2 sequences of 22G08. In another aspect, there is provided an antibody which has the CDRH1, CDRL1 and CDRL3 sequences of 22G08. In another aspect, there is provided an antibody which has the CDRH1, CDRL2 and CDRL3 sequences of 22G08. In another aspect, there is provided an antibody which has the CDRH2, CDRH3 and CDRL1 sequences of 22G08. In another aspect, there is provided an antibody which has the CDRH2, CDRL1 and CDRL2 sequences of 22G08. In another aspect, there is provided an antibody which has the CDRH2, CDRL1 and CDRL3 sequences of 22G08. In another aspect, there is provided an antibody which has the CDRH3, CDRL1 and CDRL2 sequences of 22G08. In another aspect, there is provided an antibody which has the CDRH3, CDRL1 and CDRL3 sequences of 22G08. In another aspect, there is provided an antibody which has the CDRH3, CDRL2 and CDRL3 sequences of 22G08. In another aspect, there is provided an antibody which has the CDRL1, CDRL2 and CDRL3 sequences of 22G08.

In another aspect, there is provided an antibody which has the CDRH1, CDRH2, CDRH3 and CDRL1 sequences of 22G08. In another aspect, there is provided an antibody which has the CDRH1, CDRH2, CDRH3 and CDRL2 sequences of 22G08. In another aspect, there is provided an antibody which has the CDRH1, CDRH2, CDRH3 and CDRL3 sequences of 22G08. In another aspect, there is provided an antibody which has the CDRH1, CDRH3, CDRL1 and CDRL2 sequences of 22G08. In another aspect, there is provided an antibody which has the CDRH1, CDRH3, CDRL1 and CDRL3 sequences of 22G08. In another aspect, there is provided an antibody which has the CDRH1, CDRH3, CDRL1 and CDRL2 sequences of 22G08. In another aspect, there is provided an antibody which has the CDRH1, CDRH3, CDRL1 and CDRL3 sequences of 22G08. In another aspect, there is provided an antibody which has the CDRH1, CDRL1, CDRL2 and CDRL3 sequences of 22G08. In another aspect, there is provided an antibody which has the CDRH2, CDRH3, CDRL1 and CDRL2 sequences of 22G08. In another aspect, there is provided an antibody which has the CDRH2, CDRH3, CDRL1 and CDRL3 sequences of 22G08. In another aspect, there is provided an antibody which has the CDRH2, CDRL1, CDRL2 and CDRL3 sequences of 22G08. In another aspect, there is provided an antibody which has the CDRH3, CDRL1, CDRL2 and CDRL3 sequences of 22G08.

In another aspect, there is provided an antibody which has the CDRH1, CDRH2, CDRH3, CDRL1 and CDRL2 sequences of 22G08. In another aspect, there is provided an antibody which has the CDRH1, CDRH2, CDRH3, CDRL1 and CDRL3 sequences of 22G08. In another aspect, there is provided an antibody which has the CDRH1, CDRH3, CDRL1, CDRL2 and CDRL3 sequences of 22G08. In another aspect, there is provided an antibody which has the CDRH1, CDRH2, CDRL1, CDRL2 and CDRL3 sequences of 22G08. In another aspect, there is provided an antibody which has the CDRH2, CDRH3, CDRL1, CDRL2 and CDRL3 sequences of 22G08. In another aspect, there is provided an antibody which has the CDRH1, CDRH2, CDRH3, CDRL2 and CDRL3 sequences of 22G08.

In another aspect, there is provided an antibody which has the CDRH1, CDRH2, CDRH3, CDRL1, CDRL2 and CDRL3 sequences of 22G08.

In one aspect, there is provided an antibody which has the CDRH1 and CDRH2 sequences of 22G09. In another aspect, there is provided an antibody which has the CDRH1 and CDRH3 sequences of 22G09. In another aspect, there is provided an antibody which has the CDRH1 and CDRL1 sequences of 22G09. In another aspect, there is provided an antibody which has the CDRH1 and CDRL2 sequences of 22G09. In another aspect, there is provided an antibody which has the CDRH1 and CDRL3 sequences of 22G09. In another aspect, there is provided an antibody which has the CDRH2 and CDRH3 sequences of 22G09. In another aspect, there is provided an antibody which has the CDRH2 and CDRL1 sequences of 22G09. In another aspect, there is provided an antibody which has the CDRH2 and CDRL2 sequences of 22G09. In another aspect, there is provided an antibody which has the CDRH2 and CDRL3 sequences of 22G09. In another aspect, there is provided an antibody which has the CDRH3 and CDRL1 sequences of 22G09. In another aspect, there is provided an antibody which has the CDRH3 and CDRL2 sequences of 22G09. In another aspect, there is provided an antibody which has the CDRH3 and CDRL3 sequences of 22G09. In another aspect, there is provided an antibody which has the CDRL1 and CDRL2 sequences of 22G09. In another aspect, there is provided an antibody which has the CDRL1 and CDRL3 sequences of 22G09. In another aspect, there is provided an antibody which has the CDRL2 and CDRL3 sequences of 22G09. In another aspect, there is provided an antibody which has the CDRH1, CDRH2 and CDRH3 sequences of 22G09. In another aspect, there is provided an antibody which has the CDRH1, CDRH2 and CDRL1 sequences of 22G09. In another aspect, there is provided an antibody which has the CDRH1, CDRH2 and CDRL2 sequences of 22G09. In another aspect, there is provided an antibody which has the CDRH1, CDRH2 and CDRL3 sequences of 22G09. In another aspect, there is provided an antibody which has the CDRH1, CDRH3 and CDRL1 sequences of 22G09. In another aspect, there is provided an antibody which has the CDRH1, CDRH3 and CDRL2 sequences of 22G09. In another aspect, there is provided an antibody which has the CDRH1, CDRH3 and CDRL3 sequences of 22G09. In another aspect, there is provided an antibody which has the CDRH1, CDRL1 and CDRL2 sequences of 22G09. In another aspect, there is provided an antibody which has the CDRH1, CDRL1 and CDRL3 sequences of 22G09. In another aspect, there is provided an antibody which has the CDRH1, CDRL2 and CDRL3 sequences of 22G09. In another aspect, there is provided an antibody which has the CDRH2, CDRH3 and CDRL1 sequences of 22G09. In another aspect, there is provided an antibody which has the CDRH2, CDRL1 and CDRL2 sequences of 22G09. In another aspect, there is provided an antibody which has the CDRH2, CDRL1 and CDRL3 sequences of 22G09. In another aspect, there is provided an antibody which has the CDRH3, CDRL1 and CDRL2 sequences of 22G09. In another aspect, there is provided an antibody which has the CDRH3, CDRL1 and CDRL3 sequences of 22G09. In another aspect, there is provided an antibody which has the CDRH3, CDRL2 and CDRL3 sequences of 22G09. In another aspect, there is provided an antibody which has the CDRL1, CDRL2 and CDRL3 sequences of 22G09.

In another aspect, there is provided an antibody which has the CDRH1, CDRH2, CDRH3 and CDRL1 sequences of 22G09. In another aspect, there is provided an antibody which has the CDRH1, CDRH2, CDRH3 and CDRL2 sequences of 22G09. In another aspect, there is provided an antibody which has the CDRH1, CDRH2, CDRH3 and CDRL3 sequences of 22G09. In another aspect, there is provided an antibody which has the CDRH1, CDRH3, CDRL1 and CDRL2 sequences of 22G09. In another aspect, there is provided an antibody which has the CDRH1, CDRH3, CDRL1 and CDRL3 sequences of 22G09. In another aspect, there is provided an antibody which has the CDRH1, CDRH3, CDRL1 and CDRL2 sequences of 22G09. In another aspect, there is provided an antibody which has the CDRH1, CDRH3, CDRL1 and CDRL3 sequences of 22G09. In another aspect, there is provided an antibody which has the CDRH1, CDRL1, CDRL2 and CDRL3 sequences of 22G09. In another aspect, there is provided an antibody which has the CDRH2, CDRH3, CDRL1 and CDRL2 sequences of 22G09. In another aspect, there is provided an antibody which has the CDRH2, CDRH3, CDRL1 and CDRL3 sequences of 22G09. In another aspect, there is provided an antibody which has the CDRH2, CDRL1, CDRL2 and CDRL3 sequences of 22G09. In another aspect, there is provided an antibody which has the CDRH3, CDRL1, CDRL2 and CDRL3 sequences of 22G09.

In another aspect, there is provided an antibody which has the CDRH1, CDRH2, CDRH3, CDRL1 and CDRL2 sequences of 22G09. In another aspect, there is provided an antibody which has the CDRH1, CDRH2, CDRH3, CDRL1 and CDRL3 sequences of 22G09. In another aspect, there is provided an antibody which has the CDRH1, CDRH3, CDRL1, CDRL2 and CDRL3 sequences of 22G09. In another aspect, there is provided an antibody which has the CDRH1, CDRH2, CDRL1, CDRL2 and CDRL3 sequences of 22G09. In another aspect, there is provided an antibody which has the CDRH2, CDRH3, CDRL1, CDRL2 and CDRL3 sequences of 22G09. In another aspect, there is provided an antibody which has the CDRH1, CDRH2, CDRH3, CDRL2 and CDRL3 sequences of 22G09.

In another aspect, there is provided an antibody which has the CDRH1, CDRH2, CDRH3, CDRL1, CDRL2 and CDRL3 sequences of 22G09.

In one aspect, there is provided an antibody which has the CDRH1 and CDRH2 sequences of 25A01. In another aspect, there is provided an antibody which has the CDRH1 and CDRH3 sequences of 25A01. In another aspect, there is provided an antibody which has the CDRH1 and CDRL1 sequences of 25A01. In another aspect, there is provided an antibody which has the CDRH1 and CDRL2 sequences of 25A01. In another aspect, there is provided an antibody which has the CDRH1 and CDRL3 sequences of 25A01. In another aspect, there is provided an antibody which has the CDRH2 and CDRH3 sequences of 25A01. In another aspect, there is provided an antibody which has the CDRH2 and CDRL1 sequences of 25A01. In another aspect, there is provided an antibody which has the CDRH2 and CDRL2 sequences of 25A01. In another aspect, there is provided an antibody which has the CDRH2 and CDRL3 sequences of 25A01. In another aspect, there is provided an antibody which has the CDRH3 and CDRL1 sequences of 25A01. In another aspect, there is provided an antibody which has the CDRH3 and CDRL2 sequences of 25A01. In another aspect, there is provided an antibody which has the CDRH3 and CDRL3 sequences of 25A01. In another aspect, there is provided an antibody which has the CDRL1 and CDRL2 sequences of 25A01. In another aspect, there is provided an antibody which has the CDRL1 and CDRL3 sequences of 25A01. In another aspect, there is provided an antibody which has the CDRL2 and CDRL3 sequences of 25A01. In another aspect, there is provided an antibody which has the CDRH1, CDRH2 and CDRH3 sequences of 25A01. In another aspect, there is provided an antibody which has the CDRH1, CDRH2 and CDRL1 sequences of 25A01. In another aspect, there is provided an antibody which has the CDRH1, CDRH2 and CDRL2 sequences of 25A01. In another aspect, there is provided an antibody which has the CDRH1, CDRH2 and CDRL3 sequences of 25A01. In another aspect, there is provided an antibody which has the CDRH1, CDRH3 and CDRL1 sequences of 25A01. In another aspect, there is provided an antibody which has the CDRH1, CDRH3 and CDRL2 sequences of 25A01. In another aspect, there is provided an antibody which has the CDRH1, CDRH3 and CDRL3 sequences of 25A01. In another aspect, there is provided an antibody which has the CDRH1, CDRL1 and CDRL2 sequences of 25A01. In another aspect, there is provided an antibody which has the CDRH1, CDRL1 and CDRL3 sequences of 25A01. In another aspect, there is provided an antibody which has the CDRH1, CDRL2 and CDRL3 sequences of 25A01. In another aspect, there is provided an antibody which has the CDRH2, CDRH3 and CDRL1 sequences of 25A01. In another aspect, there is provided an antibody which has the CDRH2, CDRL1 and CDRL2 sequences of 25A01. In another aspect, there is provided an antibody which has the CDRH2, CDRL1 and CDRL3 sequences of 25A01. In another aspect, there is provided an antibody which has the CDRH3, CDRL1 and CDRL2 sequences of 25A01. In another aspect, there is provided an antibody which has the CDRH3, CDRL1 and CDRL3 sequences of 25A01. In another aspect, there is provided an antibody which has the CDRH3, CDRL2 and CDRL3 sequences of 25A01. In another aspect, there is provided an antibody which has the CDRL1, CDRL2 and CDRL3 sequences of 25A01.

In another aspect, there is provided an antibody which has the CDRH1, CDRH2, CDRH3 and CDRL1 sequences of 25A01. In another aspect, there is provided an antibody which has the CDRH1, CDRH2, CDRH3 and CDRL2 sequences of 25A01. In another aspect, there is provided an antibody which has the CDRH1, CDRH2, CDRH3 and CDRL3 sequences of 25A01. In another aspect, there is provided an antibody which has the CDRH1, CDRH3, CDRL1 and CDRL2 sequences of 25A01. In another aspect, there is provided an antibody which has the CDRH1, CDRH3, CDRL1 and CDRL3 sequences of 25A01. In another aspect, there is provided an antibody which has the CDRH1, CDRH3, CDRL1 and CDRL2 sequences of 25A01. In another aspect, there is provided an antibody which has the CDRH1, CDRH3, CDRL1 and CDRL3 sequences of 25A01. In another aspect, there is provided an antibody which has the CDRH1, CDRL1, CDRL2 and CDRL3 sequences of 25A01. In another aspect, there is provided an antibody which has the CDRH2, CDRH3, CDRL1 and CDRL2 sequences of 25A01. In another aspect, there is provided an antibody which has the CDRH2, CDRH3, CDRL1 and CDRL3 sequences of 25A01. In another aspect, there is provided an antibody which has the CDRH2, CDRL1, CDRL2 and CDRL3 sequences of 25A01. In another aspect, there is provided an antibody which has the CDRH3, CDRL1, CDRL2 and CDRL3 sequences of 25A01.

In another aspect, there is provided an antibody which has the CDRH1, CDRH2, CDRH3, CDRL1 and CDRL2 sequences of 25A01. In another aspect, there is provided an antibody which has the CDRH1, CDRH2, CDRH3, CDRL1 and CDRL3 sequences of 25A01. In another aspect, there is provided an antibody which has the CDRH1, CDRH3, CDRL1, CDRL2 and CDRL3 sequences of 25A01. In another aspect, there is provided an antibody which has the CDRH1, CDRH2, CDRL1, CDRL2 and CDRL3 sequences of 25A01. In another aspect, there is provided an antibody which has the CDRH2, CDRH3, CDRL1, CDRL2 and CDRL3 sequences of 25A01. In another aspect, there is provided an antibody which has the CDRH1, CDRH2, CDRH3, CDRL2 and CDRL3 sequences of 25A014.

In another aspect, there is provided an antibody which has the CDRH1, CDRH2, CDRH3, CDRL1, CDRL2 and CDRL3 sequences of 25A01.

In one aspect, there is provided an antibody which has the CDRH1 and CDRH2 sequences of 25C01. In another aspect, there is provided an antibody which has the CDRH1 and CDRH3 sequences of 25C01. In another aspect, there is provided an antibody which has the CDRH1 and CDRL1 sequences of 25C01. In another aspect, there is provided an antibody which has the CDRH1 and CDRL2 sequences of 25C01. In another aspect, there is provided an antibody which has the CDRH1 and CDRL3 sequences of 25C01. In another aspect, there is provided an antibody which has the CDRH2 and CDRH3 sequences of 25C01. In another aspect, there is provided an antibody which has the CDRH2 and CDRL1 sequences of 25C01. In another aspect, there is provided an antibody which has the CDRH2 and CDRL2 sequences of 25C01. In another aspect, there is provided an antibody which has the CDRH2 and CDRL3 sequences of 25C01. In another aspect, there is provided an antibody which has the CDRH3 and CDRL1 sequences of 25C01. In another aspect, there is provided an antibody which has the CDRH3 and CDRL2 sequences of 25C01. In another aspect, there is provided an antibody which has the CDRH3 and CDRL3 sequences of 25C01. In another aspect, there is provided an antibody which has the CDRL1 and CDRL2 sequences of 25C01. In another aspect, there is provided an antibody which has the CDRL1 and CDRL3 sequences of 25C01. In another aspect, there is provided an antibody which has the CDRL2 and CDRL3 sequences of 25C01. In another aspect, there is provided an antibody which has the CDRH1, CDRH2 and CDRH3 sequences of 25C01. In another aspect, there is provided an antibody which has the CDRH1, CDRH2 and CDRL1 sequences of 25C01. In another aspect, there is provided an antibody which has the CDRH1, CDRH2 and CDRL2 sequences of 25C01. In another aspect, there is provided an antibody which has the CDRH1, CDRH2 and CDRL3 sequences of 25C01. In another aspect, there is provided an antibody which has the CDRH1, CDRH3 and CDRL1 sequences of 25C01. In another aspect, there is provided an antibody which has the CDRH1, CDRH3 and CDRL2 sequences of 25C01. In another aspect, there is provided an antibody which has the CDRH1, CDRH3 and CDRL3 sequences of 25C01. In another aspect, there is provided an antibody which has the CDRH1, CDRL1 and CDRL2 sequences of 25C01. In another aspect, there is provided an antibody which has the CDRH1, CDRL1 and CDRL3 sequences of 25C01. In another aspect, there is provided an antibody which has the CDRH1, CDRL2 and CDRL3 sequences of 25C01. In another aspect, there is provided an antibody which has the CDRH2, CDRH3 and CDRL1 sequences of 25C01. In another aspect, there is provided an antibody which has the CDRH2, CDRL1 and CDRL2 sequences of 25C01. In another aspect, there is provided an antibody which has the CDRH2, CDRL1 and CDRL3 sequences of 25C01. In another aspect, there is provided an antibody which has the CDRH3, CDRL1 and CDRL2 sequences of 25C01. In another aspect, there is provided an antibody which has the CDRH3, CDRL1 and CDRL3 sequences of 25C01. In another aspect, there is provided an antibody which has the CDRH3, CDRL2 and CDRL3 sequences of 25C01. In another aspect, there is provided an antibody which has the CDRL1, CDRL2 and CDRL3 sequences of 25C01.

In another aspect, there is provided an antibody which has the CDRH1, CDRH2, CDRH3 and CDRL1 sequences of 25C01. In another aspect, there is provided an antibody which has the CDRH1, CDRH2, CDRH3 and CDRL2 sequences of 25C01. In another aspect, there is provided an antibody which has the CDRH1, CDRH2, CDRH3 and CDRL3 sequences of 25C01. In another aspect, there is provided an antibody which has the CDRH1, CDRH3, CDRL1 and CDRL2 sequences of 25C01. In another aspect, there is provided an antibody which has the CDRH1, CDRH3, CDRL1 and CDRL3 sequences of 25C01. In another aspect, there is provided an antibody which has the CDRH1, CDRH3, CDRL1 and CDRL2 sequences of 25C01. In another aspect, there is provided an antibody which has the CDRH1, CDRH3, CDRL1 and CDRL3 sequences of 25C01. In another aspect, there is provided an antibody which has the CDRH1, CDRL1, CDRL2 and CDRL3 sequences of 25C01. In another aspect, there is provided an antibody which has the CDRH2, CDRH3, CDRL1 and CDRL2 sequences of 25C01. In another aspect, there is provided an antibody which has the CDRH2, CDRH3, CDRL1 and CDRL3 sequences of 25C01. In another aspect, there is provided an antibody which has the CDRH2, CDRL1, CDRL2 and CDRL3 sequences of 25C01. In another aspect, there is provided an antibody which has the CDRH3, CDRL1, CDRL2 and CDRL3 sequences of 25C01.

In another aspect, there is provided an antibody which has the CDRH1, CDRH2, CDRH3, CDRL1 and CDRL2 sequences of 25C01. In another aspect, there is provided an antibody which has the CDRH1, CDRH2, CDRH3, CDRL1 and CDRL3 sequences of 25C01. In another aspect, there is provided an antibody which has the CDRH1, CDRH3, CDRL1, CDRL2 and CDRL3 sequences of 25C01. In another aspect, there is provided an antibody which has the CDRH1, CDRH2, CDRL1, CDRL2 and CDRL3 sequences of 25C01. In another aspect, there is provided an antibody which has the CDRH2, CDRH3, CDRL1, CDRL2 and CDRL3 sequences of 25C01. In another aspect, there is provided an antibody which has the CDRH1, CDRH2, CDRH3, CDRL2 and CDRL3 sequences of 25C01.

In another aspect, there is provided an antibody which has the CDRH1, CDRH2, CDRH3, CDRL1, CDRL2 and CDRL3 sequences of 25C01.

In one aspect, there is provided an antibody which has the CDRH1 and CDRH2 sequences of 25F08. In another aspect, there is provided an antibody which has the CDRH1 and CDRH3 sequences of 25F08. In another aspect, there is provided an antibody which has the CDRH1 and CDRL1 sequences of 25F08. In another aspect, there is provided an antibody which has the CDRH1 and CDRL2 sequences of 25F08. In another aspect, there is provided an antibody which has the CDRH1 and CDRL3 sequences of 25F08. In another aspect, there is provided an antibody which has the CDRH2 and CDRH3 sequences of 25F08. In another aspect, there is provided an antibody which has the CDRH2 and CDRL1 sequences of 25F08. In another aspect, there is provided an antibody which has the CDRH2 and CDRL2 sequences of 25F08. In another aspect, there is provided an antibody which has the CDRH2 and CDRL3 sequences of 25F08. In another aspect, there is provided an antibody which has the CDRH3 and CDRL1 sequences of 25F08. In another aspect, there is provided an antibody which has the CDRH3 and CDRL2 sequences of 25F08. In another aspect, there is provided an antibody which has the CDRH3 and CDRL3 sequences of 25F08. In another aspect, there is provided an antibody which has the CDRL1 and CDRL2 sequences of 25F08. In another aspect, there is provided an antibody which has the CDRL1 and CDRL3 sequences of 25F08. In another aspect, there is provided an antibody which has the CDRL2 and CDRL3 sequences of 25F08. In another aspect, there is provided an antibody which has the CDRH1, CDRH2 and CDRH3 sequences of 25F08. In another aspect, there is provided an antibody which has the CDRH1, CDRH2 and CDRL1 sequences of 25F08. In another aspect, there is provided an antibody which has the CDRH1, CDRH2 and CDRL2 sequences of 25F08. In another aspect, there is provided an antibody which has the CDRH1, CDRH2 and CDRL3 sequences of 25F08. In another aspect, there is provided an antibody which has the CDRH1, CDRH3 and CDRL1 sequences of 25F08. In another aspect, there is provided an antibody which has the CDRH1, CDRH3 and CDRL2 sequences of 25F08. In another aspect, there is provided an antibody which has the CDRH1, CDRH3 and CDRL3 sequences of 25F08. In another aspect, there is provided an antibody which has the CDRH1, CDRL1 and CDRL2 sequences of 25F08. In another aspect, there is provided an antibody which has the CDRH1, CDRL1 and CDRL3 sequences of 25F08. In another aspect, there is provided an antibody which has the CDRH1, CDRL2 and CDRL3 sequences of 25F08. In another aspect, there is provided an antibody which has the CDRH2, CDRH3 and CDRL1 sequences of 25F08. In another aspect, there is provided an antibody which has the CDRH2, CDRL1 and CDRL2 sequences of 25F08. In another aspect, there is provided an antibody which has the CDRH2, CDRL1 and CDRL3 sequences of 25F08. In another aspect, there is provided an antibody which has the CDRH3, CDRL1 and CDRL2 sequences of 25F08. In another aspect, there is provided an antibody which has the CDRH3, CDRL1 and CDRL3 sequences of 25F08. In another aspect, there is provided an antibody which has the CDRH3, CDRL2 and CDRL3 sequences of 25F08. In another aspect, there is provided an antibody which has the CDRL1, CDRL2 and CDRL3 sequences of 25F08.

In another aspect, there is provided an antibody which has the CDRH1, CDRH2, CDRH3 and CDRL1 sequences of 25F08. In another aspect, there is provided an antibody which has the CDRH1, CDRH2, CDRH3 and CDRL2 sequences of 25F08. In another aspect, there is provided an antibody which has the CDRH1, CDRH2, CDRH3 and CDRL3 sequences of 25F08. In another aspect, there is provided an antibody which has the CDRH1, CDRH3, CDRL1 and CDRL2 sequences of 25F08. In another aspect, there is provided an antibody which has the CDRH1, CDRH3, CDRL1 and CDRL3 sequences of 25F08. In another aspect, there is provided an antibody which has the CDRH1, CDRH3, CDRL1 and CDRL2 sequences of 25F08. In another aspect, there is provided an antibody which has the CDRH1, CDRH3, CDRL1 and CDRL3 sequences of 25F08. In another aspect, there is provided an antibody which has the CDRH1, CDRL1, CDRL2 and CDRL3 sequences of 25F08. In another aspect, there is provided an antibody which has the CDRH2, CDRH3, CDRL1 and CDRL2 sequences of 25F08. In another aspect, there is provided an antibody which has the CDRH2, CDRH3, CDRL1 and CDRL3 sequences of 25F08. In another aspect, there is provided an antibody which has the CDRH2, CDRL1, CDRL2 and CDRL3 sequences of 25F08. In another aspect, there is provided an antibody which has the CDRH3, CDRL1, CDRL2 and CDRL3 sequences of 25F08.

In another aspect, there is provided an antibody which has the CDRH1, CDRH2, CDRH3, CDRL1 and CDRL2 sequences of 25F08. In another aspect, there is provided an antibody which has the CDRH1, CDRH2, CDRH3, CDRL1 and CDRL3 sequences of 25F08. In another aspect, there is provided an antibody which has the CDRH1, CDRH3, CDRL1, CDRL2 and CDRL3 sequences of 25F08. In another aspect, there is provided an antibody which has the CDRH1, CDRH2, CDRL1, CDRL2 and CDRL3 sequences of 25F08. In another aspect, there is provided an antibody which has the CDRH2, CDRH3, CDRL1, CDRL2 and CDRL3 sequences of 25F08. In another aspect, there is provided an antibody which has the CDRH1, CDRH2, CDRH3, CDRL2 and CDRL3 sequences of 25F08.

In another aspect, there is provided an antibody which has the CDRH1, CDRH2, CDRH3, CDRL1, CDRL2 and CDRL3 sequences of 25F08.

In one aspect, there is provided an antibody which has the CDRH1 and CDRH2 sequences of 28B08. In another aspect, there is provided an antibody which has the CDRH1 and CDRH3 sequences of 28B08. In another aspect, there is provided an antibody which has the CDRH1 and CDRL1 sequences of 28B08. In another aspect, there is provided an antibody which has the CDRH1 and CDRL2 sequences of 28B08. In another aspect, there is provided an antibody which has the CDRH1 and CDRL3 sequences of 28B08. In another aspect, there is provided an antibody which has the CDRH2 and CDRH3 sequences of 28B08. In another aspect, there is provided an antibody which has the CDRH2 and CDRL1 sequences of 28B08. In another aspect, there is provided an antibody which has the CDRH2 and CDRL2 sequences of 28B08. In another aspect, there is provided an antibody which has the CDRH2 and CDRL3 sequences of 28B08. In another aspect, there is provided an antibody which has the CDRH3 and CDRL1 sequences of 28B08. In another aspect, there is provided an antibody which has the CDRH3 and CDRL2 sequences of 28B08. In another aspect, there is provided an antibody which has the CDRH3 and CDRL3 sequences of 28B08. In another aspect, there is provided an antibody which has the CDRL1 and CDRL2 sequences of 28B08. In another aspect, there is provided an antibody which has the CDRL1 and CDRL3 sequences of 28B08. In another aspect, there is provided an antibody which has the CDRL2 and CDRL3 sequences of 28B08. In another aspect, there is provided an antibody which has the CDRH1, CDRH2 and CDRH3 sequences of 28B08. In another aspect, there is provided an antibody which has the CDRH1, CDRH2 and CDRL1 sequences of 28B08. In another aspect, there is provided an antibody which has the CDRH1, CDRH2 and CDRL2 sequences of 28B08. In another aspect, there is provided an antibody which has the CDRH1, CDRH2 and CDRL3 sequences of 28B08. In another aspect, there is provided an antibody which has the CDRH1, CDRH3 and CDRL1 sequences of 28B08. In another aspect, there is provided an antibody which has the CDRH1, CDRH3 and CDRL2 sequences of 28B08. In another aspect, there is provided an antibody which has the CDRH1, CDRH3 and CDRL3 sequences of 28B08. In another aspect, there is provided an antibody which has the CDRH1, CDRL1 and CDRL2 sequences of 28B08. In another aspect, there is provided an antibody which has the CDRH1, CDRL1 and CDRL3 sequences of 28B08. In another aspect, there is provided an antibody which has the CDRH1, CDRL2 and CDRL3 sequences of 28B08. In another aspect, there is provided an antibody which has the CDRH2, CDRH3 and CDRL1 sequences of 28B08. In another aspect, there is provided an antibody which has the CDRH2, CDRL1 and CDRL2 sequences of 28B08. In another aspect, there is provided an antibody which has the CDRH2, CDRL1 and CDRL3 sequences of 28B08. In another aspect, there is provided an antibody which has the CDRH3, CDRL1 and CDRL2 sequences of 28B08. In another aspect, there is provided an antibody which has the CDRH3, CDRL1 and CDRL3 sequences of 28B08. In another aspect, there is provided an antibody which has the CDRH3, CDRL2 and CDRL3 sequences of 28B08. In another aspect, there is provided an antibody which has the CDRL1, CDRL2 and CDRL3 sequences of 28B08.

In another aspect, there is provided an antibody which has the CDRH1, CDRH2, CDRH3 and CDRL1 sequences of 28B08. In another aspect, there is provided an antibody which has the CDRH1, CDRH2, CDRH3 and CDRL2 sequences of 28B08. In another aspect, there is provided an antibody which has the CDRH1, CDRH2, CDRH3 and CDRL3 sequences of 28B08. In another aspect, there is provided an antibody which has the CDRH1, CDRH3, CDRL1 and CDRL2 sequences of 28B08. In another aspect, there is provided an antibody which has the CDRH1, CDRH3, CDRL1 and CDRL3 sequences of 28B08. In another aspect, there is provided an antibody which has the CDRH1, CDRH3, CDRL1 and CDRL2 sequences of 28B08. In another aspect, there is provided an antibody which has the CDRH1, CDRH3, CDRL1 and CDRL3 sequences of 28B08. In another aspect, there is provided an antibody which has the CDRH1, CDRL1, CDRL2 and CDRL3 sequences of 28B08. In another aspect, there is provided an antibody which has the CDRH2, CDRH3, CDRL1 and CDRL2 sequences of 28B08. In another aspect, there is provided an antibody which has the CDRH2, CDRH3, CDRL1 and CDRL3 sequences of 28B08. In another aspect, there is provided an antibody which has the CDRH2, CDRL1, CDRL2 and CDRL3 sequences of 28B08. In another aspect, there is provided an antibody which has the CDRH3, CDRL1, CDRL2 and CDRL3 sequences of 28B08.

In another aspect, there is provided an antibody which has the CDRH1, CDRH2, CDRH3, CDRL1 and CDRL2 sequences of 28B08. In another aspect, there is provided an antibody which has the CDRH1, CDRH2, CDRH3, CDRL1 and CDRL3 sequences of 28B08. In another aspect, there is provided an antibody which has the CDRH1, CDRH3, CDRL1, CDRL2 and CDRL3 sequences of 28B08. In another aspect, there is provided an antibody which has the CDRH1, CDRH2, CDRL1, CDRL2 and CDRL3 sequences of 28B08. In another aspect, there is provided an antibody which has the CDRH2, CDRH3, CDRL1, CDRL2 and CDRL3 sequences of 28B08. In another aspect, there is provided an antibody which has the CDRH1, CDRH2, CDRH3, CDRL2 and CDRL3 sequences of 28B08.

In another aspect, there is provided an antibody which has the CDRH1, CDRH2, CDRH3, CDRL1, CDRL2 and CDRL3 sequences of 28B08.

In one aspect, there is provided an antibody which has the CDRH1 and CDRH2 sequences of 28C11. In another aspect, there is provided an antibody which has the CDRH1 and CDRH3 sequences of 28C11. In another aspect, there is provided an antibody which has the CDRH1 and CDRL1 sequences of 28C11. In another aspect, there is provided an antibody which has the CDRH1 and CDRL2 sequences of 28C11. In another aspect, there is provided an antibody which has the CDRH1 and CDRL3 sequences of 28C11. In another aspect, there is provided an antibody which has the CDRH2 and CDRH3 sequences of 28C11. In another aspect, there is provided an antibody which has the CDRH2 and CDRL1 sequences of 28C11. In another aspect, there is provided an antibody which has the CDRH2 and CDRL2 sequences of 28C11. In another aspect, there is provided an antibody which has the CDRH2 and CDRL3 sequences of 28C11. In another aspect, there is provided an antibody which has the CDRH3 and CDRL1 sequences of 28C11. In another aspect, there is provided an antibody which has the CDRH3 and CDRL2 sequences of 28C11. In another aspect, there is provided an antibody which has the CDRH3 and CDRL3 sequences of 28C11. In another aspect, there is provided an antibody which has the CDRL1 and CDRL2 sequences of 28C11. In another aspect, there is provided an antibody which has the CDRL1 and CDRL3 sequences of 28C11. In another aspect, there is provided an antibody which has the CDRL2 and CDRL3 sequences of 28C11. In another aspect, there is provided an antibody which has the CDRH1, CDRH2 and CDRH3 sequences of 28C11. In another aspect, there is provided an antibody which has the CDRH1, CDRH2 and CDRL1 sequences of 28C11. In another aspect, there is provided an antibody which has the CDRH1, CDRH2 and CDRL2 sequences of 28C11. In another aspect, there is provided an antibody which has the CDRH1, CDRH2 and CDRL3 sequences of 28C11. In another aspect, there is provided an antibody which has the CDRH1, CDRH3 and CDRL1 sequences of 28C11. In another aspect, there is provided an antibody which has the CDRH1, CDRH3 and CDRL2 sequences of 28C11. In another aspect, there is provided an antibody which has the CDRH1, CDRH3 and CDRL3 sequences of 28C11. In another aspect, there is provided an antibody which has the CDRH1, CDRL1 and CDRL2 sequences of 28C11. In another aspect, there is provided an antibody which has the CDRH1, CDRL1 and CDRL3 sequences of 28C11. In another aspect, there is provided an antibody which has the CDRH1, CDRL2 and CDRL3 sequences of 28C11. In another aspect, there is provided an antibody which has the CDRH2, CDRH3 and CDRL1 sequences of 28C11. In another aspect, there is provided an antibody which has the CDRH2, CDRL1 and CDRL2 sequences of 28C11. In another aspect, there is provided an antibody which has the CDRH2, CDRL1 and CDRL3 sequences of 28C11. In another aspect, there is provided an antibody which has the CDRH3, CDRL1 and CDRL2 sequences of 28C11. In another aspect, there is provided an antibody which has the CDRH3, CDRL1 and CDRL3 sequences of 28C11. In another aspect, there is provided an antibody which has the CDRH3, CDRL2 and CDRL3 sequences of 28C11. In another aspect, there is provided an antibody which has the CDRL1, CDRL2 and CDRL3 sequences of 28C11.

In another aspect, there is provided an antibody which has the CDRH1, CDRH2, CDRH3 and CDRL1 sequences of 28C11. In another aspect, there is provided an antibody which has the CDRH1, CDRH2, CDRH3 and CDRL2 sequences of 28C11. In another aspect, there is provided an antibody which has the CDRH1, CDRH2, CDRH3 and CDRL3 sequences of 28C11. In another aspect, there is provided an antibody which has the CDRH1, CDRH3, CDRL1 and CDRL2 sequences of 28C11. In another aspect, there is provided an antibody which has the CDRH1, CDRH3, CDRL1 and CDRL3 sequences of 28C11. In another aspect, there is provided an antibody which has the CDRH1, CDRH3, CDRL1 and CDRL2 sequences of 28C11. In another aspect, there is provided an antibody which has the CDRH1, CDRH3, CDRL1 and CDRL3 sequences of 28C11. In another aspect, there is provided an antibody which has the CDRH1, CDRL1, CDRL2 and CDRL3 sequences of 28C11. In another aspect, there is provided an antibody which has the CDRH2, CDRH3, CDRL1 and CDRL2 sequences of 28C11. In another aspect, there is provided an antibody which has the CDRH2, CDRH3, CDRL1 and CDRL3 sequences of 28C11. In another aspect, there is provided an antibody which has the CDRH2, CDRL1, CDRL2 and CDRL3 sequences of 28C11. In another aspect, there is provided an antibody which has the CDRH3, CDRL1, CDRL2 and CDRL3 sequences of 28C11.

In another aspect, there is provided an antibody which has the CDRH1, CDRH2, CDRH3, CDRL1 and CDRL2 sequences of 28C11. In another aspect, there is provided an antibody which has the CDRH1, CDRH2, CDRH3, CDRL1 and CDRL3 sequences of 28C11. In another aspect, there is provided an antibody which has the CDRH1, CDRH3, CDRL1, CDRL2 and CDRL3 sequences of 28C11. In another aspect, there is provided an antibody which has the CDRH1, CDRH2, CDRL1, CDRL2 and CDRL3 sequences of 28C11. In another aspect, there is provided an antibody which has the CDRH2, CDRH3, CDRL1, CDRL2 and CDRL3 sequences of 28C11. In another aspect, there is provided an antibody which has the CDRH1, CDRH2, CDRH3, CDRL2 and CDRL3 sequences of 28C11.

In another aspect, there is provided an antibody which has the CDRH1, CDRH2, CDRH3, CDRL1, CDRL2 and CDRL3 sequences of 28C11.

In one aspect, there is provided an antibody which has the CDRH1 and CDRH2 sequences of 32B04. In another aspect, there is provided an antibody which has the CDRH1 and CDRH3 sequences of 32B04. In another aspect, there is provided an antibody which has the CDRH1 and CDRL1 sequences of 32B04. In another aspect, there is provided an antibody which has the CDRH1 and CDRL2 sequences of 32B04. In another aspect, there is provided an antibody which has the CDRH1 and CDRL3 sequences of 32B04. In another aspect, there is provided an antibody which has the CDRH2 and CDRH3 sequences of 32B04. In another aspect, there is provided an antibody which has the CDRH2 and CDRL1 sequences of 32B04. In another aspect, there is provided an antibody which has the CDRH2 and CDRL2 sequences of 32B04. In another aspect, there is provided an antibody which has the CDRH2 and CDRL3 sequences of 32B04. In another aspect, there is provided an antibody which has the CDRH3 and CDRL1 sequences of 32B04. In another aspect, there is provided an antibody which has the CDRH3 and CDRL2 sequences of 32B04. In another aspect, there is provided an antibody which has the CDRH3 and CDRL3 sequences of 32B04. In another aspect, there is provided an antibody which has the CDRL1 and CDRL2 sequences of 32B04. In another aspect, there is provided an antibody which has the CDRL1 and CDRL3 sequences of 32B04. In another aspect, there is provided an antibody which has the CDRL2 and CDRL3 sequences of 32B04. In another aspect, there is provided an antibody which has the CDRH1, CDRH2 and CDRH3 sequences of 32B04. In another aspect, there is provided an antibody which has the CDRH1, CDRH2 and CDRL1 sequences of 32B04. In another aspect, there is provided an antibody which has the CDRH1, CDRH2 and CDRL2 sequences of 32B04. In another aspect, there is provided an antibody which has the CDRH1, CDRH2 and CDRL3 sequences of 32B04. In another aspect, there is provided an antibody which has the CDRH1, CDRH3 and CDRL1 sequences of 32B04. In another aspect, there is provided an antibody which has the CDRH1, CDRH3 and CDRL2 sequences of 32B04. In another aspect, there is provided an antibody which has the CDRH1, CDRH3 and CDRL3 sequences of 32B04. In another aspect, there is provided an antibody which has the CDRH1, CDRL1 and CDRL2 sequences of 32B04. In another aspect, there is provided an antibody which has the CDRH1, CDRL1 and CDRL3 sequences of 32B04. In another aspect, there is provided an antibody which has the CDRH1, CDRL2 and CDRL3 sequences of 32B04. In another aspect, there is provided an antibody which has the CDRH2, CDRH3 and CDRL1 sequences of 32B04. In another aspect, there is provided an antibody which has the CDRH2, CDRL1 and CDRL2 sequences of 32B04. In another aspect, there is provided an antibody which has the CDRH2, CDRL1 and CDRL3 sequences of 32B04. In another aspect, there is provided an antibody which has the CDRH3, CDRL1 and CDRL2 sequences of 32B04. In another aspect, there is provided an antibody which has the CDRH3, CDRL1 and CDRL3 sequences of 32B04. In another aspect, there is provided an antibody which has the CDRH3, CDRL2 and CDRL3 sequences of 32B04. In another aspect, there is provided an antibody which has the CDRL1, CDRL2 and CDRL3 sequences of 32B04.

In another aspect, there is provided an antibody which has the CDRH1, CDRH2, CDRH3 and CDRL1 sequences of 32B04. In another aspect, there is provided an antibody which has the CDRH1, CDRH2, CDRH3 and CDRL2 sequences of 32B04. In another aspect, there is provided an antibody which has the CDRH1, CDRH2, CDRH3 and CDRL3 sequences of 32B04. In another aspect, there is provided an antibody which has the CDRH1, CDRH3, CDRL1 and CDRL2 sequences of 32B04. In another aspect, there is provided an antibody which has the CDRH1, CDRH3, CDRL1 and CDRL3 sequences of 32B04. In another aspect, there is provided an antibody which has the CDRH1, CDRH3, CDRL1 and CDRL2 sequences of 32B04. In another aspect, there is provided an antibody which has the CDRH1, CDRH3, CDRL1 and CDRL3 sequences of 32B04. In another aspect, there is provided an antibody which has the CDRH1, CDRL1, CDRL2 and CDRL3 sequences of 32B04. In another aspect, there is provided an antibody which has the CDRH2, CDRH3, CDRL1 and CDRL2 sequences of 32B04. In another aspect, there is provided an antibody which has the CDRH2, CDRH3, CDRL1 and CDRL3 sequences of 32B04. In another aspect, there is provided an antibody which has the CDRH2, CDRL1, CDRL2 and CDRL3 sequences of 32B04. In another aspect, there is provided an antibody which has the CDRH3, CDRL1, CDRL2 and CDRL3 sequences of 32B04.

In another aspect, there is provided an antibody which has the CDRH1, CDRH2, CDRH3, CDRL1 and CDRL2 sequences of 32B04. In another aspect, there is provided an antibody which has the CDRH1, CDRH2, CDRH3, CDRL1 and CDRL3 sequences of 32B04. In another aspect, there is provided an antibody which has the CDRH1, CDRH3, CDRL1, CDRL2 and CDRL3 sequences of 32B04. In another aspect, there is provided an antibody which has the CDRH1, CDRH2, CDRL1, CDRL2 and CDRL3 sequences of 32B04. In another aspect, there is provided an antibody which has the CDRH2, CDRH3, CDRL1, CDRL2 and CDRL3 sequences of 32B04. In another aspect, there is provided an antibody which has the CDRH1, CDRH2, CDRH3, CDRL2 and CDRL3 sequences of 32B04.

In another aspect, there is provided an antibody which has the CDRH1, CDRH2, CDRH3, CDRL1, CDRL2 and CDRL3 sequences of 32B04.

In one aspect, there is provided an antibody which has the CDRH1 and CDRH2 sequences of 32D04. In another aspect, there is provided an antibody which has the CDRH1 and CDRH3 sequences of 32D04. In another aspect, there is provided an antibody which has the CDRH1 and CDRL1 sequences of 32D04. In another aspect, there is provided an antibody which has the CDRH1 and CDRL2 sequences of 32D04. In another aspect, there is provided an antibody which has the CDRH1 and CDRL3 sequences of 32D04. In another aspect, there is provided an antibody which has the CDRH2 and CDRH3 sequences of 32D04. In another aspect, there is provided an antibody which has the CDRH2 and CDRL1 sequences of 32D04. In another aspect, there is provided an antibody which has the CDRH2 and CDRL2 sequences of 32D04. In another aspect, there is provided an antibody which has the CDRH2 and CDRL3 sequences of 32D04. In another aspect, there is provided an antibody which has the CDRH3 and CDRL1 sequences of 32D04. In another aspect, there is provided an antibody which has the CDRH3 and CDRL2 sequences of 32D04. In another aspect, there is provided an antibody which has the CDRH3 and CDRL3 sequences of 32D04. In another aspect, there is provided an antibody which has the CDRL1 and CDRL2 sequences of 32D04. In another aspect, there is provided an antibody which has the CDRL1 and CDRL3 sequences of 32D04. In another aspect, there is provided an antibody which has the CDRL2 and CDRL3 sequences of 32D04. In another aspect, there is provided an antibody which has the CDRH1, CDRH2 and CDRH3 sequences of 32D04. In another aspect, there is provided an antibody which has the CDRH1, CDRH2 and CDRL1 sequences of 32D04. In another aspect, there is provided an antibody which has the CDRH1, CDRH2 and CDRL2 sequences of 32D04. In another aspect, there is provided an antibody which has the CDRH1, CDRH2 and CDRL3 sequences of 32D04. In another aspect, there is provided an antibody which has the CDRH1, CDRH3 and CDRL1 sequences of 32D04. In another aspect, there is provided an antibody which has the CDRH1, CDRH3 and CDRL2 sequences of 32D04. In another aspect, there is provided an antibody which has the CDRH1, CDRH3 and CDRL3 sequences of 32D04. In another aspect, there is provided an antibody which has the CDRH1, CDRL1 and CDRL2 sequences of 32D04. In another aspect, there is provided an antibody which has the CDRH1, CDRL1 and CDRL3 sequences of 32D04. In another aspect, there is provided an antibody which has the CDRH1, CDRL2 and CDRL3 sequences of 32D04. In another aspect, there is provided an antibody which has the CDRH2, CDRH3 and CDRL1 sequences of 32D04. In another aspect, there is provided an antibody which has the CDRH2, CDRL1 and CDRL2 sequences of 32D04. In another aspect, there is provided an antibody which has the CDRH2, CDRL1 and CDRL3 sequences of 32D04. In another aspect, there is provided an antibody which has the CDRH3, CDRL1 and CDRL2 sequences of 32D04. In another aspect, there is provided an antibody which has the CDRH3, CDRL1 and CDRL3 sequences of 32D04. In another aspect, there is provided an antibody which has the CDRH3, CDRL2 and CDRL3 sequences of 32D04. In another aspect, there is provided an antibody which has the CDRL1, CDRL2 and CDRL3 sequences of 32D04.

In another aspect, there is provided an antibody which has the CDRH1, CDRH2, CDRH3 and CDRL1 sequences of 32D04. In another aspect, there is provided an antibody which has the CDRH1, CDRH2, CDRH3 and CDRL2 sequences of 32D04. In another aspect, there is provided an antibody which has the CDRH1, CDRH2, CDRH3 and CDRL3 sequences of 32D04. In another aspect, there is provided an antibody which has the CDRH1, CDRH3, CDRL1 and CDRL2 sequences of 32D04. In another aspect, there is provided an antibody which has the CDRH1, CDRH3, CDRL1 and CDRL3 sequences of 32D04. In another aspect, there is provided an antibody which has the CDRH1, CDRH3, CDRL1 and CDRL2 sequences of 32D04. In another aspect, there is provided an antibody which has the CDRH1, CDRH3, CDRL1 and CDRL3 sequences of 32D04. In another aspect, there is provided an antibody which has the CDRH1, CDRL1, CDRL2 and CDRL3 sequences of 32D04. In another aspect, there is provided an antibody which has the CDRH2, CDRH3, CDRL1 and CDRL2 sequences of 32D04. In another aspect, there is provided an antibody which has the CDRH2, CDRH3, CDRL1 and CDRL3 sequences of 32D04. In another aspect, there is provided an antibody which has the CDRH2, CDRL1, CDRL2 and CDRL3 sequences of 32D04. In another aspect, there is provided an antibody which has the CDRH3, CDRL1, CDRL2 and CDRL3 sequences of 32D04.

In another aspect, there is provided an antibody which has the CDRH1, CDRH2, CDRH3, CDRL1 and CDRL2 sequences of 32D04. In another aspect, there is provided an antibody which has the CDRH1, CDRH2, CDRH3, CDRL1 and CDRL3 sequences of 32D04. In another aspect, there is provided an antibody which has the CDRH1, CDRH3, CDRL1, CDRL2 and CDRL3 sequences of 32D04. In another aspect, there is provided an antibody which has the CDRH1, CDRH2, CDRL1, CDRL2 and CDRL3 sequences of 32D04. In another aspect, there is provided an antibody which has the CDRH2, CDRH3, CDRL1, CDRL2 and CDRL3 sequences of 32D04. In another aspect, there is provided an antibody which has the CDRH1, CDRH2, CDRH3, CDRL2 and CDRL3 sequences of 32D04.

In another aspect, there is provided an antibody which has the CDRH1, CDRH2, CDRH3, CDRL1, CDRL2 and CDRL3 sequences of 32D04.

In one aspect, there is provided an antibody which has the CDRH1 and CDRH2 sequences of 35A06. In another aspect, there is provided an antibody which has the CDRH1 and CDRH3 sequences of 35A06. In another aspect, there is provided an antibody which has the CDRH1 and CDRL1 sequences of 35A06. In another aspect, there is provided an antibody which has the CDRH1 and CDRL2 sequences of 35A06. In another aspect, there is provided an antibody which has the CDRH1 and CDRL3 sequences of 35A06. In another aspect, there is provided an antibody which has the CDRH2 and CDRH3 sequences of 35A06. In another aspect, there is provided an antibody which has the CDRH2 and CDRL1 sequences of 35A06. In another aspect, there is provided an antibody which has the CDRH2 and CDRL2 sequences of 35A06. In another aspect, there is provided an antibody which has the CDRH2 and CDRL3 sequences of 35A06. In another aspect, there is provided an antibody which has the CDRH3 and CDRL1 sequences of 35A06. In another aspect, there is provided an antibody which has the CDRH3 and CDRL2 sequences of 35A06. In another aspect, there is provided an antibody which has the CDRH3 and CDRL3 sequences of 35A06. In another aspect, there is provided an antibody which has the CDRL1 and CDRL2 sequences of 35A06. In another aspect, there is provided an antibody which has the CDRL1 and CDRL3 sequences of 35A06. In another aspect, there is provided an antibody which has the CDRL2 and CDRL3 sequences of 35A06. In another aspect, there is provided an antibody which has the CDRH1, CDRH2 and CDRH3 sequences of 35A06. In another aspect, there is provided an antibody which has the CDRH1, CDRH2 and CDRL1 sequences of 35A06. In another aspect, there is provided an antibody which has the CDRH1, CDRH2 and CDRL2 sequences of 35A06. In another aspect, there is provided an antibody which has the CDRH1, CDRH2 and CDRL3 sequences of 35A06. In another aspect, there is provided an antibody which has the CDRH1, CDRH3 and CDRL1 sequences of 35A06. In another aspect, there is provided an antibody which has the CDRH1, CDRH3 and CDRL2 sequences of 35A06. In another aspect, there is provided an antibody which has the CDRH1, CDRH3 and CDRL3 sequences of 35A06. In another aspect, there is provided an antibody which has the CDRH1, CDRL1 and CDRL2 sequences of 35A06. In another aspect, there is provided an antibody which has the CDRH1, CDRL1 and CDRL3 sequences of 35A06. In another aspect, there is provided an antibody which has the CDRH1, CDRL2 and CDRL3 sequences of 35A06. In another aspect, there is provided an antibody which has the CDRH2, CDRH3 and CDRL1 sequences of 35A06. In another aspect, there is provided an antibody which has the CDRH2, CDRL1 and CDRL2 sequences of 35A06. In another aspect, there is provided an antibody which has the CDRH2, CDRL1 and CDRL3 sequences of 35A06. In another aspect, there is provided an antibody which has the CDRH3, CDRL1 and CDRL2 sequences of 35A06. In another aspect, there is provided an antibody which has the CDRH3, CDRL1 and CDRL3 sequences of 35A06. In another aspect, there is provided an antibody which has the CDRH3, CDRL2 and CDRL3 sequences of 35A06. In another aspect, there is provided an antibody which has the CDRL1, CDRL2 and CDRL3 sequences of 35A06.

In another aspect, there is provided an antibody which has the CDRH1, CDRH2, CDRH3 and CDRL1 sequences of 35A06. In another aspect, there is provided an antibody which has the CDRH1, CDRH2, CDRH3 and CDRL2 sequences of 35A06. In another aspect, there is provided an antibody which has the CDRH1, CDRH2, CDRH3 and CDRL3 sequences of 35A06. In another aspect, there is provided an antibody which has the CDRH1, CDRH3, CDRL1 and CDRL2 sequences of 35A06. In another aspect, there is provided an antibody which has the CDRH1, CDRH3, CDRL1 and CDRL3 sequences of 35A06. In another aspect, there is provided an antibody which has the CDRH1, CDRH3, CDRL1 and CDRL2 sequences of 35A06. In another aspect, there is provided an antibody which has the CDRH1, CDRH3, CDRL1 and CDRL3 sequences of 35A06. In another aspect, there is provided an antibody which has the CDRH1, CDRL1, CDRL2 and CDRL3 sequences of 35A06. In another aspect, there is provided an antibody which has the CDRH2, CDRH3, CDRL1 and CDRL2 sequences of 35A06. In another aspect, there is provided an antibody which has the CDRH2, CDRH3, CDRL1 and CDRL3 sequences of 35A06. In another aspect, there is provided an antibody which has the CDRH2, CDRL1, CDRL2 and CDRL3 sequences of 35A06. In another aspect, there is provided an antibody which has the CDRH3, CDRL1, CDRL2 and CDRL3 sequences of 35A06.

In another aspect, there is provided an antibody which has the CDRH1, CDRH2, CDRH3, CDRL1 and CDRL2 sequences of 35A06. In another aspect, there is provided an antibody which has the CDRH1, CDRH2, CDRH3, CDRL1 and CDRL3 sequences of 35A06. In another aspect, there is provided an antibody which has the CDRH1, CDRH3, CDRL1, CDRL2 and CDRL3 sequences of 35A06. In another aspect, there is provided an antibody which has the CDRH1, CDRH2, CDRL1, CDRL2 and CDRL3 sequences of 35A06. In another aspect, there is provided an antibody which has the CDRH2, CDRH3, CDRL1, CDRL2 and CDRL3 sequences of 35A06. In another aspect, there is provided an antibody which has the CDRH1, CDRH2, CDRH3, CDRL2 and CDRL3 sequences of 35A0622D04.

In another aspect, there is provided an antibody which has the CDRH1, CDRH2, CDRH3, CDRL1, CDRL2 and CDRL3 sequences of 35A06.

In one aspect, there is provided an antibody which has the CDRH1 and CDRH2 sequences of 35E11. In another aspect, there is provided an antibody which has the CDRH1 and CDRH3 sequences of 35E11. In another aspect, there is provided an antibody which has the CDRH1 and CDRL1 sequences of 35E11. In another aspect, there is provided an antibody which has the CDRH1 and CDRL2 sequences of 35E11. In another aspect, there is provided an antibody which has the CDRH1 and CDRL3 sequences of 35E11. In another aspect, there is provided an antibody which has the CDRH2 and CDRH3 sequences of 35E11. In another aspect, there is provided an antibody which has the CDRH2 and CDRL1 sequences of 35E11. In another aspect, there is provided an antibody which has the CDRH2 and CDRL2 sequences of 35E11. In another aspect, there is provided an antibody which has the CDRH2 and CDRL3 sequences of 35E11. In another aspect, there is provided an antibody which has the CDRH3 and CDRL1 sequences of 35E11. In another aspect, there is provided an antibody which has the CDRH3 and CDRL2 sequences of 35E11. In another aspect, there is provided an antibody which has the CDRH3 and CDRL3 sequences of 35E11. In another aspect, there is provided an antibody which has the CDRL1 and CDRL2 sequences of 35E11. In another aspect, there is provided an antibody which has the CDRL1 and CDRL3 sequences of 35E11. In another aspect, there is provided an antibody which has the CDRL2 and CDRL3 sequences of 35E11. In another aspect, there is provided an antibody which has the CDRH1, CDRH2 and CDRH3 sequences of 35E11. In another aspect, there is provided an antibody which has the CDRH1, CDRH2 and CDRL1 sequences of 35E11. In another aspect, there is provided an antibody which has the CDRH1, CDRH2 and CDRL2 sequences of 35E11. In another aspect, there is provided an antibody which has the CDRH1, CDRH2 and CDRL3 sequences of 35E11. In another aspect, there is provided an antibody which has the CDRH1, CDRH3 and CDRL1 sequences of 35E11. In another aspect, there is provided an antibody which has the CDRH1, CDRH3 and CDRL2 sequences of 35E11. In another aspect, there is provided an antibody which has the CDRH1, CDRH3 and CDRL3 sequences of 35E11. In another aspect, there is provided an antibody which has the CDRH1, CDRL1 and CDRL2 sequences of 35E11. In another aspect, there is provided an antibody which has the CDRH1, CDRL1 and CDRL3 sequences of 35E11. In another aspect, there is provided an antibody which has the CDRH1, CDRL2 and CDRL3 sequences of 35E11. In another aspect, there is provided an antibody which has the CDRH2, CDRH3 and CDRL1 sequences of 35E11. In another aspect, there is provided an antibody which has the CDRH2, CDRL1 and CDRL2 sequences of 35E11. In another aspect, there is provided an antibody which has the CDRH2, CDRL1 and CDRL3 sequences of 35E11. In another aspect, there is provided an antibody which has the CDRH3, CDRL1 and CDRL2 sequences of 35E11. In another aspect, there is provided an antibody which has the CDRH3, CDRL1 and CDRL3 sequences of 35E11. In another aspect, there is provided an antibody which has the CDRH3, CDRL2 and CDRL3 sequences of 35E11. In another aspect, there is provided an antibody which has the CDRL1, CDRL2 and CDRL3 sequences of 35E11.

In another aspect, there is provided an antibody which has the CDRH1, CDRH2, CDRH3 and CDRL1 sequences of 35E11. In another aspect, there is provided an antibody which has the CDRH1, CDRH2, CDRH3 and CDRL2 sequences of 35E11. In another aspect, there is provided an antibody which has the CDRH1, CDRH2, CDRH3 and CDRL3 sequences of 35E11. In another aspect, there is provided an antibody which has the CDRH1, CDRH3, CDRL1 and CDRL2 sequences of 35E11. In another aspect, there is provided an antibody which has the CDRH1, CDRH3, CDRL1 and CDRL3 sequences of 35E11. In another aspect, there is provided an antibody which has the CDRH1, CDRH3, CDRL1 and CDRL2 sequences of 35E11. In another aspect, there is provided an antibody which has the CDRH1, CDRH3, CDRL1 and CDRL3 sequences of 35E11. In another aspect, there is provided an antibody which has the CDRH1, CDRL1, CDRL2 and CDRL3 sequences of 35E11. In another aspect, there is provided an antibody which has the CDRH2, CDRH3, CDRL1 and CDRL2 sequences of 35E11. In another aspect, there is provided an antibody which has the CDRH2, CDRH3, CDRL1 and CDRL3 sequences of 35E11. In another aspect, there is provided an antibody which has the CDRH2, CDRL1, CDRL2 and CDRL3 sequences of 35E11. In another aspect, there is provided an antibody which has the CDRH3, CDRL1, CDRL2 and CDRL3 sequences of 35E11.

In another aspect, there is provided an antibody which has the CDRH1, CDRH2, CDRH3, CDRL1 and CDRL2 sequences of 35E11. In another aspect, there is provided an antibody which has the CDRH1, CDRH2, CDRH3, CDRL1 and CDRL3 sequences of 35E11. In another aspect, there is provided an antibody which has the CDRH1, CDRH3, CDRL1, CDRL2 and CDRL3 sequences of 35E11. In another aspect, there is provided an antibody which has the CDRH1, CDRH2, CDRL1, CDRL2 and CDRL3 sequences of 35E11. In another aspect, there is provided an antibody which has the CDRH2, CDRH3, CDRL1, CDRL2 and CDRL3 sequences of 35E11. In another aspect, there is provided an antibody which has the CDRH1, CDRH2, CDRH3, CDRL2 and CDRL3 sequences of 35E11.

In another aspect, there is provided an antibody which has the CDRH1, CDRH2, CDRH3, CDRL1, CDRL2 and CDRL3 sequences of 35E11.

Sentence 9. An antibody or fragment according to any one of sentences 1 to 8, which has an isotype selected from IgG, IgE, IgM, IgD and IgA.

Sentence 10. An antibody or fragment according to sentence 9, which has an isotype selected from IgG1, IgG2, IgG3 and IgG4, for example, the isotype is IgG1 or IgG4, and optionally is IgG2a or IgG2c.

Sentence 11. An antibody or fragment according to any one of sentences 1 to 10 which further comprises a heavy chain constant region which is IgG4-PE (Seq ID No: 602).

Sentence 12. An antibody or fragment which binds to the same epitope of human Nav1.7 (SEQ ID No:2) as an antibody as defined in any one of sentences 1 to 11.

Sentence 13. An antibody or fragment which competes for binding to human Nav1.7 (SEQ ID No:2) with an antibody as defined in any one of sentences 1 to 11, optionally as measured by SPR or ELISA.

Sentence 14. An antibody or fragment thereof according to any one of sentences 1 to 13 which binds to human NAV1.7 (SEQ ID No:2) with an IC₅₀ of less than 100 nM, and/or a degree of maximum inhibition of at least 50%.

Sentence 15. An antibody or fragment according sentence 14, wherein the IC₅₀ is less than 50 nM, less than 10 nM, less than 5 nM, less than 1 nM, less than 0.5 nM, less than 100 pM, less than 50 pM, less than 20 pM, less than 10 pM or less than 1 pM.

Sentence 16. An antibody or fragment according to sentence 15 wherein the IC₅₀ is less than 100 pM.

Sentence 17. An antibody or fragment according to any one of sentences 14 to 16, where in the degree of maximum inhibition is at least 55%, at least 60%, at least 65%, at least 70%, at least 75%, at least 80%, at least 85%, at least 90%, at least 92%, at least 95%, at least 97% or is 100%.

Sentence 18. An antibody or fragment according to any one of sentences 14 to 27, wherein the IC₅₀ and/or % maximum inhibition are determined according to a standard whole cell patch clamp assay.

Sentence 19. An antibody or fragment according to sentence 18, wherein the patch clamp assay is performed as described in Example 2F, Example 2G, Example 2H or Example 2I.

Sentence 20. An antibody or fragment according to sentence 18, wherein the patch clamp assay comprises the following steps:
a. Providing cells (e.g. HEK293 cell) expressing (e.g. stably expressing) a human NAV1.7 protein;
b. Taking baseline electrophysiological recordings by measuring ion currents (e.g. by ion current measurements in the perforated patch clamp configuration (for example with 200 μgml-1 amphoterocin) at room temperature (for example at 21-23° C.) using an IonWorks Quattro instrument in population patch clamp (PPC) mode), optionally wherein the cells are clamped at a holding potential of −90 mV for 30 s and then repeatedly stepped to 0 mV for 20 ms at a frequency of 10 Hz, and wherein currents are measured from the 1st and 25th steps and referenced to the holding current, and optionally wherein the internal solution contains (mM): 90 K gluconate, 40 KCl, 10 NaCl, 3.2 MgCl2, 3.2 EGTA, 5 HEPES and is buffered to pH 7.3, and the external solution contains (mM): 137 NaCl, 4 KCl, 1.8 CaCl₂, 1 MgCl₂, 10 HEPES also buffered to pH 7.3;
c. contacting said cells with a test antibody (e.g. for 5 to 7 minutes);
d. And repeating step b. to obtain a second measurement using an identical pulse train; and
e. Comparing the measurements taken in step b. and step d. to obtain IC₅₀ and/or degree of maximum inhibition values; and optionally
f. Repeating steps c., d. and e. with a positive control (such as a known toxin, e.g. tetracaine or ProTxII) instead of a test antibody to obtain IC₅₀ and/or degree of maximum inhibition values for said positive control.

Sentence 21. An antibody or fragment according to any one of sentences 14 to 20, wherein the IC₅₀ is calculated according to the formula:

IC₅₀=half maximal inhibitory concentration=concentration of antibody required for 50% inhibition

Sentence 22. An antibody or fragment according to any one of sentences 14 to 21, wherein the degree of maximum inhibition is calculated according to the formula:

Maximum inhibition (Max) (%)=maximal inhibition induced by antibody

Sentence 23. An antibody or fragment according to any one of sentences 1 to 22, wherein the antibody or fragment has an affinity (Kd) of less than 100 nM and optionally wherein the affinity is measured using an SPR method on a NAV1.7 polypeptide, wherein the SPR method comprises the following steps:
a. Coupling anti-mouse (or other relevant human, rat or non-human vertebrate antibody constant region species-matched) IgG (e.g., Biacore™ BR-1008-38) to a biosensor chip (e.g., GLM chip) such as by primary amine coupling;
b. Exposing the anti-mouse IgG (or other matched species antibody) to a test IgG antibody to capture test antibody on the chip;
c. Passing the test antigen over the chip's capture surface at 1024 nM, 256 nM, 64 nM, 16 nM, 4 nM with a 0 nM (i.e. buffer alone); and
d. And determining the affinity of binding of test antibody to test antigen using surface plasmon resonance, e.g., at 25° C. or at 37° C., optionally in physiological buffer such as a buffer at pH7.6, comprising 150 mM NaCl, 0.05% detergent (e.g., Polysorbate 20) and 3 mM EDTA, or a buffer containing 10 mM Hepes, or a buffer which is HBS-EP.

Sentence 24. An antibody or fragment according to sentence 23, wherein the antibody or fragment has an affinity (Kd) of less than 50 nM, less than 10 nM, less than 1 nM, less than 500 pM or less than 100 pM.

Sentence 25. An antibody fragment according to any one of sentences 1 to 24, wherein the fragment is selected from a Fab, a Fab′, a F(ab′)2, a bispecific Fab, a dsFv, a camelized V_{H}, a bispecific scFv, a diabody, a triabody and a scFv.

Sentence 26. An antibody or fragment according to any one of sentences 1 to 25 which is monoclonal.

Sentence 27. An antibody or fragment according to any one of sentences 1 to 26 wherein the antibody or fragment binds a NAV1.7 protein at 25° C. and acidic pH with a dissociative half-life (t½) of less than about 4.5 minutes (such as less than about 2 minutes, e.g. less than about 1.5 minutes), and wherein the antibody or fragment binds a NAV protein at 25° C. and neutral pH with a t½ of greater than about 35 minutes.

Sentence 28. A nucleic acid encoding an antibody or fragment as defined in any one of sentences 1 to 27.

Sentence 29. A nucleic acid that encodes a VH domain and/or a VL domain of an antibody or fragment as defined in any one of sentences 1 to 27.

Sentence 30. A nucleic acid that encodes a CHRH1, CDRH2, CDRH3, CDRL1, CDRL2 and/or CDRL3 (for example CHRH1, CDRH2 and/or CDRH3; or CDRL1, CDRL2 and/or CDRL3, in particular either CDRH3 or CDRL3) of an antibody or fragment as defined in any one of sentences 1 to 27.

Sentence 31. A nucleic acid that encodes a heavy chain or a light chain of an antibody or fragment as defined in any one of sentences 1 to 27.

Sentence 32. A vector comprising the nucleic acid as defined in any one of sentences 29 to 31, optionally wherein the vector is a CHO or HEK293 vector.

Sentence 33. A host cell comprising the nucleic acid as defined in any one of sentences 29 to 31 or the vector as defined in claim 32.

Sentence 34. A host cell according to sentence 33, which is selected from CHO, HEK (e.g. HEK293), NSO, and COS (e.g. COS7).

Sentence 35. A hybridoma expressing an antibody or fragment as defined in any one of sentences 1 to 27.

Sentence 36. A pharmaceutical composition comprising an antibody or fragment as defined in any one of sentences 1 to 27, and a diluent, excipient or carrier.

Sentence 37. A pharmaceutical composition according to sentence 36, further comprising an anti-nociceptive drug.

Sentence 38. A pharmaceutical composition for use in treating, preventing and/or reducing the risk of a NAV1.7-mediated condition or disease, the composition comprising an antibody or fragment as defined in any one of sentences 1 to 27, and a diluent, excipient or carrier; and optionally further comprising an anti-nociceptive drug.

Sentence 39. A pharmaceutical composition according to sentence 37 or sentence 38, wherein the anti-nociceptive drug is selected from an opioid analgesic (e.g. morphine, diamorphine, codeine, dihydrocodeine, fentanyl, oxycodone, buprenorphine, dextropropoxyphene, tramadol, meptazinol, pethidine or pantazocine), paracetamol, a non-steroidal anti-inflammatory (e.g. aspirin, ibuprofen, ketoprofen, naproxen, indomethacin, diclofenac, celecoxib, ketorolac, mefenamic acid, meloxicam, piroxicam, nabumetone, parecoxib, sulindac or tenoxicam), a local anaesthetic (e.g. bupivacaine, lignocaine), a 5HT1 agonist (e.g. sumptriptan or naratriptan), an anti-epileptic/antidepressant (e.g. carbamazepine, gabapentin, pregabalin or duloxetine), an anxiolytic/muscle relaxant (e.g. diazepam, tizanidine or cyclobenzaprine), ziconitide, botulinum toxin, tetrahydrocannabinol, cannabidiol, capsaicin, an anti-NGF drug, and anti-TrkA drug, an anti-CGRP drug, p75NTR-Fc, a COX-1 antagonist, a COX-2 antagonist, a TRPV1 antagonist, a TRPV3 agonist, a voltage-gated sodium channel blocker or a FAAH inhibitor.

Sentence 40. An antibody or fragment as defined in any one of sentences 1 to 27 for use in therapy.

Sentence 41. An antibody or fragment as defined in any one of sentences 1 to 27 for use in the treatment and/or prevention of a NAV1.7-mediated disease or condition.

Sentence 42. Use of an antibody or fragment as defined in any one of sentences 1 to 27 in the manufacture of a medicament for administration to a human, for treating and/or preventing a NAV1.7-mediated disease or condition in the human.

Sentence 43. A method of treating and/or preventing and/or reducing the risk of a NAV1.7-mediated disease or condition in a human by administering to said human a therapeutically effective amount of an antibody or fragment as defined in any one of sentences 1 to 27.

Sentence 44. The antibody or fragment for the use as defined in sentence 41 or the use of the antibody or fragment as defined in sentence 42 or the method as defined in sentence 43, wherein the NAV1.7-mediated disease or condition is selected from:
a. Neuropathic/neurogenic pain (for example arising from painful diabetic neuropathy (PDN), post-herpetic neuropathy (PHN), central neuropathy, peripheral neuropathy, trigeminal neuralgia (TN), anaesthesia dolorosa, spinal cord injuries, multiple sclerosis, phantom limb pain, hyperalgesia, hyperpathia, paresthesia, psychogenic pain, post-stroke pain and HIV-associated pain, back pain, chronic back pain, osteoarthritis, cancer, breakthrough pain, erythromelalgia [e.g. primary erythromelalgia], paroxysmal extreme pain disorder, nerve compression and/or entrapment [such as carpal tunnel syndrome, tarsal tunnel syndrome, ulnar nerve entrapment, compression radiculopathy, radicular low back pain, spinal root lesions, spinal root compression, lumbar spinal stenosis, sciatic nerve compression, intercostal neuralgia], neuritis, pain from chemotherapy, congenital defect/channelopathy [e.g. channelopathy-associated insensitivity to pain and congenital insensitivity to pain], chronic alcoholism [alcoholic polyneuropathy]);
b. inflammation (such as osteoarthritis, chronic back pain, rheumatoid arthritis, cancer, breakthrough pain, burns, encephalitis, bone fracture, neuritis, autoimmune diseases, postoperative pain, dental pain, bacterial infection, radiotherapy, gout and irritable bowel syndrome);
c. pain from trauma (such as from lacerations, incisions, burns, foreign bodies or bullet and/or shrapnel injuries, spinal cord injury, brachial plexus avulsion, nerve crush and/or entrapment (such as carpal tunnel syndrome, tarsal tunnel syndrome, ulnar nerve entrapment, compression radiculopathy, radicular low back pain, spinal root lesions, spinal root compression, lumbar spinal stenosis, sciatic nerve compression, intercostal neuralgia), nerve transection, post-operative pain, dental pain and toxic exposure);
d. pain from infection (such as post-herpetic neuropathy (PHN), HIV-associated pain small pox infection, encephalitis, herpes infection, and bacterial infection);
e. pain from malignancy (such as cancer pain, breakthrough pain, and nerve compression pain);
f. visceral pain (such as renal/ureteral colic, irritable bowel syndrome, angina/cardiac pain, cardiac arrhythmia, period pain, interstitial cystitis, rectal pain, pain associated with diarrhoea, appendicitis, cholecystitis and pancreatitis);
g. metabolic/chronic disease (such as multiple sclerosis, cancer pain, breakthrough pain, gout, peripheral diabetic neuropathy, chronic alcoholism [alcoholic polyneuropathy], uremia, hypothyroidism and vitamin deficiency);
h. headache pain (such as tension headache, migraine and cluster headaches);
i. idiopathic pain (such as trigeminal neuralgia, complex regional pain syndromes [e.g. complex regional pain syndrome I and complex regional pain syndrome II], allodynia and fibromyalgia);
j. respiratory pain (such as pain associated with asthma, airway hyper-reactivity in asthma, chronic cough, e.g. in asthma and/or chronic obstructive pulmonary disorder); or
k. other pain (such as pain associated with hormonal therapy, diabetes, hypothyroidism, epilepsy, ataxia, periodic paralysis, acute itch and chronic itch).

Sentence 45. The antibody, use or method according to sentence 44, wherein the NAV1.7-mediated disease or condition is selected from painful diabetic neuropathy, post-herpetic neuropathy, trigeminal neuralgia, osteoarthritis, chronic back pain, nerve compression pain (e.g. sciatic nerve compression) or cancer pain; or is selected from migraine, post-operative pain and fibromyalgia.

### Other Physical Characteristics of Anti-NAV Antibodies Described Herein

As discussed above antibodies and antibody fragments, as disclosed herein in embodiment, or combination of embodiments may take a variety of formats. Any discussion with respect to antibodies applies mutatis mutandis to antibody fragments of the invention.

The antibodies as disclosed herein, from any embodiment, or combination of embodiments, may be from any animal origin including birds and mammals (e.g., human, murine, donkey, sheep, rabbit, goat, guinea pig, camel, horse, or chicken) and in one embodiment are of human or mouse origin. In certain embodiments, the antibodies as disclosed herein are chimeric, humanised, or fully human antibodies. In another embodiment, the antibodies are recombinant fully human antibodies.

In one embodiment, the antibodies as disclosed herein are chimeric antibodies, which may be chimeric antibodies comprising human variable domains and non-human constant regions. Such non-human constant regions may be derived from a number of non-human species, such as rodent, rat, mouse, human, rabbit, chicken, Camelid, sheep, bovine, non-human primate or shark. In one embodiment, the non-human constant region is derived from a rodent, such as a mouse or a rat, e.g. a mouse. In a further embodiment, the constant region is expressed from an Ig locus comprising mouse 129 strain constant regions.

In another embodiment, the antibodies as disclosed herein comprise human variable regions. In another embodiment, the antibodies as disclosed herein comprise human variable regions which comprise mouse pattern terminal deoxynucleotidyl transferase (TdT) mutation, in particular a mouse 129 strain TdT mutation.

In a particular embodiment, the antibodies as disclosed herein are fully human antibodies, such as fully human antibodies that bind a NAV protein polypeptide, a NAV protein polypeptide fragment, or a NAV protein epitope, such as an epitope from one or two of the external loop regions (e.g. from one or two of the D1E1, D1E2, D1E3, D2E1, D2E2, D2E3, D3E1, D3E2, D3E3, D4E1, D4E2 and the D4E3 loop regions). Such fully human antibodies would be advantageous over fully mouse (or other full or partial non-human species antibodies), humanized antibodies, or chimeric antibodies to minimize the development of unwanted or unneeded side effects, such as immune responses directed toward non-fully human antibodies) when administered to the subject.

The antibodies as disclosed herein may be monospecific, bispecific, trispecific or of greater multispecificity, e.g. bispecific or trispecific. Multispecific antibodies may bind to different epitopes of a NAV protein polypeptide or may be specific for both a NAV protein polypeptide as well as for a heterologous epitope, such as a heterologous polypeptide or solid support material. In another embodiment, the multispecific (e.g. bispecific or trispecific) antibodies as disclosed herein bind an epitope on a molecule selected from Calcitonin Gene-Related Peptide (CGRP), Nerve growth factor (NGF), Transient Receptor Potential Cation Channel subfamily V Member 1 (TRPV1), Transient Receptor Potential Cation Channel subfamily V Member 3 (TRPV3), TRK1-transforming tyrosine kinase protein (TRK-A), human p75 neurotrophin receptor (p75NTR) and Fatty Acid Amide Hydrolase (FAAH). In a particular embodiment, the multispecific (e.g. bispecific or trispecific) antibodies as disclosed herein bind an epitope on a molecule selected from CGRP, NGF, TRPV1 and TRK-A.

In certain embodiments, the antibodies disclosed herein are monoclonal antibodies. In other embodiments, the antibodies disclosed herein are isolated monoclonal antibodies. In other embodiments, the antibodies disclosed herein are recombinant, monoclonal antibodies.

In some embodiments, the antibodies provided herein bind to a NAV protein epitope wherein the binding to the NAV protein epitope by the antibody is competitively blocked (e.g., in a dose-dependent manner) by a toxin selected from tetrodotoxin (TTX), saxitoxin (STX), hanatoxin, centipede toxin, μ-SLPTX-Ssm6a, Protoxin-I (ProTx-I), Protoxin-II (ProTx-II), Huwentoxin-IV (HwTx-IV) and a conotoxin (such as μ-GIIIA, μ-GIIIB, μ-GIIIC, μ-PIIIA, μ-TIIIA, μ-SmIIIA, μ-KIIIA, μ-SIIIA, μ-CoIIIA, μ-CoIIIB, μ-CIIIA, μ-MIIIA, μO-MrVIA, μO-MrVIB, δ-TxVIA, δ-TxVIB, δGmVIA, δ-PVIA, δ-NgVIA, δ-EVIA and δ-SVIE, in particular μ-KIIIA).

In certain embodiments, an antibody is provided herein that binds to a NAV protein epitope wherein the binding to the NAV protein epitope by the antibody is competitively blocked (e.g., in a dose-dependent manner) by an antibody or fragment of the invention. The antibody may or may not be a fully human antibody. In preferred embodiments, the antibody is a fully human monoclonal anti-NAV protein antibody, and even more preferably a fully human, monoclonal, antagonistic anti-NAV protein antibody. Exemplary competitive blocking tests that can be used are known to those skilled in the art.

Preferably, the antibodies are fully human, monoclonal antibodies, such as fully human, monoclonal antagonist antibodies, that bind to NAV protein.

In certain embodiments, the anti-NAV protein antibody comprises less than six CDRs. In some embodiments, the anti-NAV protein antibody comprises or consists of one, two, three, four, or five CDRs selected from the group consisting of HCDR1, HCDR2, HCDR3, LCDR1, LCDR2, and LCDR3.

In an example, the antibody or fragment is a lambda-type antibody or fragment (i.e., whose variable domains are lambda variable domains). Optionally, the antibody or fragment also comprises lambda constant domains.

There are provided antibodies that bind to a NAV protein antigen which comprise a framework region known to those of skill in the art (e.g., a human or non-human fragment). The framework region may, for example, be naturally occurring or consensus framework regions. Most preferably, the framework region of an anti-NAV protein antibody as disclosed herein is human (see, e.g., Chothia et al., 1998, J. Mol. Biol. 278:457-479 for a listing of human framework regions, which is incorporated by reference herein in its entirety). See also Kabat et al. (1991) Sequences of Proteins of Immunological Interest (U.S. Department of Health and Human Services, Washington, D.C.) 5th ed.

The antibodies as disclosed herein include antibodies that are chemically modified, i.e., by the covalent attachment of any type of molecule to the antibody. For example, but not by way of limitation, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formulation, metabolic synthesis of tunicamycin, etc. Additionally, the antibody may contain one or more non-classical amino acids.

### Determination of Biological Properties

For all the anti-NAV protein antibodies described herein in any embodiment, or combination of embodiments, biological properties may be determined in a number of assays, known to those skilled in the art.

In one aspect, the antibodies provided herein bind to a NAV protein of interest (e.g. hNAV1.7, hNAV1.8 and/or hNAV1.9). The potency of binding to the NAV protein of interest may be determined with functional NAV proteins or polypeptides in terms of IC₅₀. Alternatively, the efficacy of binding to the NAV protein of interest may be determined with functional NAV proteins or polypeptides in terms of degree of maximum inhibition. Alternatively, the binding affinity to the NAV protein of interest may be determined with NAV polypeptides, e.g. loop polypeptides or fragments thereof, by SPR, e.g. by Biacore.

The antibodies disclosed herein (in any of the embodiments or combination of embodiments) bind the NAV protein polypeptides of interest, such as hNAV1.7, hNAV1.8 and/or hNAV1.9 protein polypeptides, in particular in an external loop polypeptides selected from D1E1, D1E2, D1E3, D2E1, D2E2, D2E3, D3E1, D3E2, D3E3, D4E1, D4E2 and D4E3 or fragments thereof (e.g., selected from D1E2, D2E2, D3E2 and D4E2 loop region polypeptides, for example any of SEQ ID No: 4, 8, 12, 16, 22, 26, 30, 34, 40, 44, 48, 52, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328) with a potency (IC₅₀) of less than 100 nM, less than 50 nM, less than 10 nM, less than 5 nM, less than 1 nM, less than 0.5 nM, less than 100 pM, less than 50 pM, less than 20 pM, less than 10 pM or less than 1 pM. The IC₅₀ may be determined in a number of functional assays, e.g. in a standard whole cell Patch Clamp (PC) assay, for example under PC conditions disclosed herein). In another embodiment, the antibodies disclosed herein bind the NAV protein polypeptides of interest, with a potency (IC₅₀) of less than 100 nM. In another embodiment, the antibodies disclosed herein bind the NAV protein polypeptides of interest, with a potency (IC₅₀) of less than 50 nM. In another embodiment, the antibodies disclosed herein bind the NAV protein polypeptides of interest, with a potency (IC₅₀) of less than 10 nM. In another embodiment, the antibodies disclosed herein bind the NAV protein polypeptides of interest, with a potency (IC₅₀) of less than 100 pM. In another embodiment, the antibodies disclosed herein bind the NAV protein polypeptides of interest, with a potency (IC₅₀) of less than 50 pM. In another embodiment, the antibodies disclosed herein bind the NAV protein polypeptides of interest, with a potency (IC₅₀) of less than 10 pM. In one embodiment, the IC₅₀ is calculated according to the following formula:

IC₅₀=half maximal inhibitory concentration=concentration of antibody required for 50% inhibition

The antibodies disclosed herein (in any of the embodiments or combination of embodiments) bind the NAV protein polypeptides of interest, such as hNAV1.7, hNAV1.8 and/or hNAV1.9 protein polypeptides, in particular in an external loop polypeptides selected from D1E1, D1E2, D1E3, D2E1, D2E2, D2E3, D3E1, D3E2, D3E3, D4E1, D4E2 and D4E3 or fragments thereof (e.g., selected from D1E2, D2E2, D3E2 and D4E2 loop region polypeptides, for example SEQ ID No: 4, 8, 12, 16, 22, 26, 30, 34, 40, 44, 48, 52, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328) with an efficacy (degree of maximum inhibition) which is at least 50%, at least 55%, at least 60%, at least 65%, at least 70%, at least 75%, at least 80%, at least 85%, at least 90%, at least 92%, at least 95%, at least 97% or is 100% The degree of maximum inhibition may be determined in a number of functional assays known to those skilled in the art, e.g. in a standard whole cell Patch Clamp (PC) assay, for example under PC conditions disclosed herein). In another embodiment, the antibodies disclosed herein bind the NAV protein polypeptides of interest, with an efficacy (degree of maximum inhibition) which is at least 50%. In another embodiment, the antibodies disclosed herein bind the NAV protein polypeptides of interest, with an efficacy (degree of maximum inhibition) which is at least 70%. In another embodiment, the antibodies disclosed herein bind the NAV protein polypeptides of interest, with an efficacy (degree of maximum inhibition) which is at least 80%. In another embodiment, the antibodies disclosed herein bind the NAV protein polypeptides of interest, with an efficacy (degree of maximum inhibition) which is at least 90%. In another embodiment, the antibodies disclosed herein bind the NAV protein polypeptides of interest, with an efficacy (degree of maximum inhibition) which is at least 95%. In another embodiment, the antibodies disclosed herein bind the NAV protein polypeptides of interest, with an efficacy (degree of maximum inhibition) which is 100%. In one embodiment, the degree of maximum inhibition is calculated according to the formula:

Maximum inhibition (Max) (%)=maximal inhibition induced by antibody

Such potency and/or efficacy measurements can be made using a variety of binding assays known in the art, e.g. using an IonWorks® Quattro (IWQ) Patch Clamp Device (as described in Example 2G), using a Port-a-Patch® Patch Clamp Device (as described in Example 2H below), using a Q-Patch Clamp Device (as described in Example 2I below) or generally in a standard whole cell PC assay as described in Example 2F below.

In one embodiment, the standard whole cell PC assay is as described in sentence 317:

Sentence 317. An antibody or fragment as described herein (in any of the embodiments or combination of embodiments) wherein the patch clamp assay comprises the following steps:
a. Providing cells expressing a NAV protein of interest;
b. Taking baseline electrophysiological recordings by measuring ion currents;
c. contacting said cells with a test antibody;
d. And repeating step b. to obtain a second measurement using an identical pulse train; and
e. Comparing the measurements taken in step b. and step d. to obtain IC₅₀ and/or degree of maximum inhibition values; and optionally
f. Repeating steps c., d. and e. with a positive control instead of a test antibody to obtain IC₅₀ and/or degree of maximum inhibition values for said positive control.

In one embodiment the cells are HEK293 cells. In another embodiment, the cells stably express the NAV protein of interest. In a further embodiment, the cells are HEK293 cells which stably express the NAV protein of interest (e.g. full length hNAV1.7, full length hNAV1.8 and/or full length hNAV1.9).

In one embodiment, the baseline recordings of step b. are ion current measurements in the perforated patch clamp configuration (for example with 200 μgml-1 amphoterocin) using an IonWorks Quattro instrument in population patch clamp (PPC) mode). In another embodiment, the cells are clamped at a holding potential of −90 mV for 30 s and then repeatedly stepped to 0 mV for 20 ms at a frequency of 10 Hz. In a further embodiment, the currents are measured from the 1st and 25th steps and referenced to the holding current.

In another embodiment, the PC assay comprises an internal solution, which contains (mM): 90 K gluconate, 40 KCl, 10 NaCl, 3.2 MgCl₂, 3.2 EGTA, 5 HEPES and is buffered to pH 7.3. In another embodiment, the PC assay comprises an external solution, which contains (mM): 137 NaCl, 4 KCl, 1.8 CaCl₂, 1 MgCl₂, 10 HEPES also buffered to pH 7.3

In another embodiment, the ion current measurements (of step b. and/or step d.) are recorded at room temperature, for example at 21-23° C.

In a further embodiment, the test antibody is contacted with the cells for about 5 to 7 minutes.

In another embodiment, the positive control is a known toxin, e.g. tetracaine or ProTxII.

The antibodies disclosed herein (in any of the embodiments or combination of embodiments) bind the NAV protein polypeptides of interest, such as hNAV1.7, hNAV1.8 and/or hNAV1.9 protein polypeptides, in particular in an external loop polypeptides selected from D1E1, D1E2, D1E3, D2E1, D2E2, D2E3, D3E1, D3E2, D3E3, D4E1, D4E2 and D4E3 or fragments thereof (e.g., selected from D1E2, D2E2, D3E2 and D4E2 loop region polypeptides, for example SEQ ID No: 4, 8, 12, 16, 22, 26, 30, 34, 40, 44, 48, 52, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328) with an affinity (apparent affinity, Kd) of less than 1 mM, 1000 nM to 100 nM, 100 nM to 10 nM, 10 nM to 1 nM, 1000 pM to 500 pM, 500 pM to 200 pM, less than 200 pM, 200 pM to 150 pM, 200 pM to 100 pM, 100 pM to 10 pM, 10 pM to 1 pM, e.g., in the range of 1 mM to 1 pM (e.g., 1 mM to 100 pM; 10 nM to 100 pM; 1 nM to 10 pM; or 100 pM to 1 pM) as determined by SPR, e.g., under SPR conditions disclosed herein). In another embodiment, the antibodies disclosed herein bind the NAV protein polypeptides of interest, such as hNAV1.7, hNAV1.8 and/or hNAV1.9 protein polypeptides, in particular in an external loop polypeptides selected from D1E1, D1E2, D1E3, D2E1, D2E2, D2E3, D3E1, D3E2, D3E3, D4E1, D4E2 and D4E3 or fragments thereof (e.g., selected from D1E2, D2E2, D3E2 and D4E2 loop region polypeptides, for example SEQ ID No: 4, 8, 12, 16, 22, 26, 30, 34, 40, 44, 48, 52, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328) with an affinity (apparent affinity, Kd) of less than 10 pM, less than 1 pM, less than 100 nM, less than 10 nM, less than 1 nM, less than 100 pM, less than 10 pM, or less that 1 pM (e.g. less than 10 nM or less than 1 nM).

Such binding measurements can be made using a variety of binding assays known in the art, e.g., using surface plasmon resonance (SPR), such as by Biacore™ or using the ProteOn XPR36™ (Bio-Rad®), or using KinExA® (Sapidyne Instruments, Inc).

Binding affinity (Kd, K_{off} and/or Kₒₙ) can be determined by any routine method in the art, eg, by surface plasmon resonance (SPR).

In one embodiment, the surface plasmon resonance (SPR) is carried out at 25° C. In another embodiment, the SPR is carried out at 37° C.

In one embodiment, the SPR is carried out at physiological pH, such as about pH7 or at pH7.6 (e.g., using Hepes buffered saline at pH7.6 (also referred to as HBS-EP)).

In one embodiment, the SPR is carried out at a physiological salt level, e.g., 150 mM NaCl.

In one embodiment, the SPR is carried out at a detergent level of no greater than 0.05% by volume, e.g., in the presence of P20 (polysorbate 20; e.g., Tween-20m) at 0.05% and EDTA at 3 mM.

In one example, the SPR is carried out at 25° C. or 37° C. in a buffer at pH7.6, 150 mM NaCl, 0.05% detergent (e.g., P20) and 3 mM EDTA. The buffer can contain 10 mM Hepes. In one example, the SPR is carried out at 25° C. or 37° C. in HBS-EP. HBS-EP is available from Teknova Inc (California; catalogue number H8022).

In an example, the affinity of the antibody or fragment is determined using SPR by
1. Coupling anti-mouse (or other relevant human, rat or non-human vertebrate antibody constant region species-matched) IgG (e.g., Biacore™ BR-1008-38) to a biosensor chip (e.g., GLM chip) such as by primary amine coupling;
2. Exposing the anti-mouse IgG (or other matched species antibody) to a test IgG antibody to capture test antibody on the chip;
3. Passing the test antigen over the chip's capture surface at 1024 nM, 256 nM, 64 nM, 16 nM, 4 nM with a 0 nM (i.e. buffer alone); and
4. And determining the affinity of binding of test antibody to test antigen using surface plasmon resonance, e.g., under an SPR condition discussed above (e.g., at 25° C. in physiological buffer). SPR can be carried out using any standard SPR apparatus, such as by Biacore™ or using the ProteOn XPR36™ (Bio-Rad®).

Regeneration of the capture surface can be carried out with 10 mM glycine at pH1.7. This removes the captured antibody and allows the surface to be used for another interaction. The binding data can be fitted to 1:1 model inherent using standard techniques, e.g., using a model inherent to the ProteOn XPR36™ analysis software.

As discussed above, the anti-NAV protein antibodies as disclosed herein may be selective over other NAV proteins of interest (e.g. an anti-hNAV1.7 antibody may be selective over one, two or all of hNAV1.4, hNAV1.5 and hNAV1.6). A given anti-NAV protein antibody is considered to be selective when it has an improved binding affinity, efficacy or potency (e.g. as measured by Kd, degree of maximum inhibition or IC₅₀) over the other NAV protein of interest.

The binding affinity (e.g. Kd) is considered to be selective, in one embodiment, when the affinity for a first NAV polypeptide is at least 10-fold, 20-fold, 30-fold, 50-fold, 100-fold, 200-fold, 500-fold, 1000-fold or 1,500-fold greater than the affinity for a second NAV polypeptide (e.g. when the affinity for an external loop region polypeptide of NAV1.7 is 100-fold greater than the affinity to an external loop region polypeptide of NAV14, NAV1.5 and/or NAV1.6). In another embodiment, the binding affinity (e.g. Kd) is considered to be selective when the affinity for a first NAV polypeptide is at least 50-fold, 100-fold, 200-fold, 500-fold, 1000-fold or 1,500-fold greater than the affinity for a second NAV polypeptide. In another embodiment, the binding affinity (e.g. Kd) is considered to be selective when the affinity for a first NAV polypeptide is at least 200-fold, 500-fold, 1000-fold or 1,500-fold greater than the affinity for a second NAV polypeptide. In another embodiment, the binding affinity (e.g. Kd) is considered to be selective when the affinity for a first NAV polypeptide is at least 500-fold greater than the affinity for a second NAV polypeptide. In another embodiment, the binding affinity (e.g. Kd) is considered to be selective when the affinity for a first NAV polypeptide is at least 1000-fold greater than the affinity for a second NAV polypeptide. In another embodiment, the binding affinity (e.g. Kd) is considered to be selective when the affinity for a first NAV polypeptide is at least 1,500-fold greater than the affinity for a second NAV polypeptide. In another embodiment, the binding affinity (e.g. Kd) is considered to be selective when the affinity for a first NAV polypeptide is at least 10-fold, at least a 25-fold, at least 50-fold, at least 100-fold, at least 150-fold, at least 200-fold, at least 300-fold, at least 400-fold, at least 500-fold, at least 700-fold or at least 1000-fold greater than the affinity for a second NAV polypeptide. In another embodiment, the binding affinity (e.g. Kd) is considered to be selective when the affinity for a first NAV polypeptide is at least 200-fold, at least 300-fold, at least 400-fold, at least 500-fold, at least 700-fold or at least 1000-fold greater than the affinity for a second NAV polypeptide. In another embodiment, the binding affinity (e.g. Kd) is considered to be selective when the affinity for a first NAV polypeptide is at least at least 400-fold, at least 500-fold, at least 700-fold or at least 1000-fold greater than the affinity for a second NAV polypeptide.

The potency (e.g. IC₅₀) is considered to be selective, in one embodiment, when the IC₅₀ of a first NAV protein is at least 10-fold, 20-fold, 30-fold, 50-fold, 100-fold, 200-fold, 500-fold, 1000-fold or 1,500-fold greater than the IC₅₀ of a second NAV protein (e.g. when the IC₅₀ of a NAV1.7 protein is 100-fold greater than the IC₅₀ of a NAV1.4, NAV1.5 and/or NAV1.6 protein). In another embodiment, the potency (e.g. IC₅₀) is considered to be selective when the IC₅₀ of a first NAV protein is at least 50-fold, 100-fold, 200-fold, 500-fold, 1000-fold or 1,500-fold greater than the IC₅₀ of a second NAV protein. In another embodiment, the potency (e.g. IC₅₀) is considered to be selective when the IC₅₀ of a first NAV protein is at least 200-fold, 500-fold, 1000-fold or 1,500-fold greater than the IC₅₀ of a second NAV protein. In another embodiment, the potency (e.g. IC₅₀) is considered to be selective when the IC₅₀ of a first NAV protein is at least 500-fold greater than the IC₅₀ of a second NAV protein. In another embodiment, the potency (e.g. IC₅₀) is considered to be selective when the IC₅₀ of a first NAV protein is at least 1000-fold greater than the IC₅₀ of a second NAV protein. In another embodiment, the potency (e.g. IC₅₀) is considered to be selective when the IC₅₀ of a first NAV protein is at least 1,500-fold greater than the IC₅₀ of a second NAV protein. In another embodiment, the potency (e.g. IC₅₀) is considered to be selective when the IC₅₀ for a first NAV polypeptide is at least 10-fold, at least a 25-fold, at least 50-fold, at least 100-fold, at least 150-fold, at least 200-fold, at least 300-fold, at least 400-fold, at least 500-fold, at least 700-fold or at least 1000-fold greater than the IC₅₀ for a second NAV polypeptide. In another embodiment, the potency (e.g. IC₅₀) is considered to be selective when the IC₅₀ for a first NAV polypeptide is at least 200-fold, at least 300-fold, at least 400-fold, at least 500-fold, at least 700-fold or at least 1000-fold greater than the IC₅₀ for a second NAV polypeptide. In another embodiment, the potency (e.g. IC₅₀) is considered to be selective when the IC₅₀ for a first NAV polypeptide is at least at least 400-fold, at least 500-fold, at least 700-fold or at least 1000-fold greater than the IC₅₀ for a second NAV polypeptide.

The efficacy (e.g. degree of maximum inhibition) is considered to be selective, in one embodiment, when the degree of maximum inhibition of a first NAV protein is at least 10-fold, 20-fold, 30-fold, 50-fold, 100-fold, 200-fold, 500-fold, 1000-fold or 1,500-fold greater than the degree of maximum inhibition of a second NAV protein (e.g. when the degree of maximum inhibition of a NAV1.7 protein is 100-fold greater than the degree of maximum inhibition of a NAV14, NAV1.5 and/or NAV1.6 protein). In another embodiment, the efficacy (e.g. degree of maximum inhibition) is considered to be selective when the degree of maximum inhibition of a first NAV protein is at least 50-fold, 100-fold, 200-fold, 500-fold, 1000-fold or 1,500-fold greater than the degree of maximum inhibition of a second NAV protein. In another embodiment, the efficacy (e.g. degree of maximum inhibition) is considered to be selective when the degree of maximum inhibition of a first NAV protein is at least 200-fold, 500-fold, 1000-fold or 1,500-fold greater than the degree of maximum inhibition of a second NAV protein. In another embodiment, the efficacy (e.g. degree of maximum inhibition) is considered to be selective when the degree of maximum inhibition of a first NAV protein is at least 500-fold greater than the degree of maximum inhibition of a second NAV protein. In another embodiment, the efficacy (e.g. degree of maximum inhibition) is considered to be selective when the degree of maximum inhibition of a first NAV protein is at least 1000-fold greater than the degree of maximum inhibition of a second NAV protein. In another embodiment, the efficacy (e.g. degree of maximum inhibition) is considered to be selective when the degree of maximum inhibition of a first NAV protein is at least 1,500-fold greater than the degree of maximum inhibition of a second NAV protein. In another embodiment, the efficacy (e.g. degree of maximum inhibition) is considered to be selective when the degree of maximum inhibition for a first NAV protein is at least 10-fold, at least a 25-fold, at least 50-fold, at least 100-fold, at least 150-fold, at least 200-fold, at least 300-fold, at least 400-fold, at least 500-fold, at least 700-fold or at least 1000-fold greater than the degree of maximum inhibition for a second NAV protein. In another embodiment, the efficacy (e.g. degree of maximum inhibition) is considered to be selective when the degree of maximum inhibition for a first NAV protein is at least 200-fold, at least 300-fold, at least 400-fold, at least 500-fold, at least 700-fold or at least 1000-fold greater than the degree of maximum inhibition for a second NAV protein. In another embodiment, the efficacy (e.g. degree of maximum inhibition) is considered to be selective when the degree of maximum inhibition for a first NAV protein is at least at least 400-fold, at least 500-fold, at least 700-fold or at least 1000-fold greater than the degree of maximum inhibition for a second NAV protein.

As discussed above, the anti-NAV protein antibodies as disclosed herein may be cross reactive with other NAV proteins of interest from the same species (e.g. an anti-hNAV1.7 antibody may be cross reactive with either or both or hNAV1.8 and hNAV1.9). A given anti-NAV protein antibody is considered to be cross reactive when it has a comparable binding affinity, efficacy or potency (e.g. as Kd, degree of maximum inhibition or IC₅₀) to the other NAV protein of interest from the same species.

The binding affinity, potency and/or efficacy is considered to be comparable, in one embodiment, when the affinity, potency and/or efficacy of a first and second NAV protein or polypeptide are within at least 1.5-fold, at least 2-fold, at least 3-fold, at least 5-fold, at least 7-fold, at least 10-fold, at least 15-fold, at least 20-fold or at least 30-fold of each other. In another embodiment, the affinity, potency and/or efficacy are within at least 1.5-fold, at least 2-fold, at least 3-fold or at least 5-fold of each other.

The affinity is considered to be comparable, in another embodiment, when the affinity is measured by Kd, and is less than 10 nM, less than 7 nM, less than 5 nM, less than 3 nM or less than 1 nM (such as less the 5 nM, e.g. less than 1 nM) for both a first NAV protein polypeptide and for a second NAV protein polypeptide. In another embodiment, the affinity is considered to be comparable when the affinity is measured by Kd, and is less than 100 nM, less than 50 nM, less than 10 nM, less than 5 nM, less than 1 nM, less than 0.5 nM, less than 100 pM, less than 50 pM, less than 20 pM, less than 10 pM or less than 1 pM (such as less the 100 pM, e.g. less than 20 pM) for both the first polypeptide and for the second polypeptide. In one embodiment, the first and second polypeptide are extracellular loop polypeptides of two different NAV proteins of interest (e.g. an extracellular loop region of hNAV1.7 and an extracellular loop region of hNAV1.8 or hNAV1.9, e.g. the E2 loop region).

The potency is considered to be comparable, in another embodiment, when the potency is measured by IC₅₀, and is less than 10 nM, less than 7 nM, less than 5 nM, less than 3 nM or less than 1 nM (such as less the 5 nM, e.g. less than 1 nM) for both the first polypeptide and for the second polypeptide. In another embodiment, the potency is considered to be comparable when the potency is measured by IC₅₀, and is less than 100 nM, less than 50 nM, less than 10 nM, less than 5 nM, less than 1 nM, less than 0.5 nM, less than 100 pM, less than 50 pM, less than 20 pM, less than 10 pM or less than 1 pM (such as less the 100 pM, e.g. less than 20 pM) for both the first polypeptide and for the second polypeptide. In one embodiment, the first and second polypeptide are full length, NAV proteins of interest (e.g. full length hNAV1.7 and full length hNAV1.8 or hNAV1.9). In another embodiment, the first and second polypeptide are full length, NAV proteins of interest expressed on the surface of a cell.

The efficacy is considered to be comparable, in another embodiment, when the efficacy is measured by degree of maximum inhibition, and is greater than 50%, greater than 60%, greater than 70%, greater than 80%, greater than 90% or greater than 95% (such as greater than 70%, e.g. greater than 80%) for both the first polypeptide and for the second polypeptide. In another embodiment, the efficacy is considered to be comparable when the efficacy is measured by degree of maximum inhibition, and is greater than 80%, greater than 90%, greater than 95% (such as greater than 90%, e.g. greater than 95%) for both the first polypeptide and for the second polypeptide. In one embodiment, the first and second polypeptide are full length, NAV proteins of interest (e.g. full length hNAV1.7 and full length hNAV1.8 or hNAV1.9). In another embodiment, the first and second polypeptide are full length, NAV proteins of interest expressed on the surface of a cell.

### Methods of Producing Antibodies

Antibodies that bind to a NAV protein antigen (as disclosed herein in any embodiment, or combination of embodiments) can be produced by any method known in the art for the synthesis of antibodies, in particular, by chemical synthesis or preferably, by recombinant expression techniques. Unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art may be used to produce the antibodies disclosed herein. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, N.Y.; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

Polyclonal antibodies that bind to an antigen can be produced by various procedures well-known in the art. For example, a human antigen can be administered to various host animals including, but not limited to, rabbits, mice, rats, etc. to induce the production of sera containing polyclonal antibodies specific for the human antigen.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981) (said references incorporated by reference in their entireties). Other exemplary methods of producing monoclonal antibodies are discussed elsewhere herein, such as e.g., use of the KM Mouse™. Additional exemplary methods of producing monoclonal antibodies are provided in the Examples herein.

Antibody fragments which recognize NAV protein antigens may be generated by any technique known to those of skill in the art. For example, Fab and F(ab′)₂ fragments may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain. Further, the anti-NAV protein antibodies as disclosed herein can also be generated using various phage display methods known in the art.

For example, antibodies can also be generated using various phage display methods. Alternatively, fully human antibodies may be generated through the in vitro screening of phage display antibody libraries; see e.g., Hoogenboom et al., J. Mol. Biol., 227:381 (1991); Marks et al., J. Mol. Biol., 222:581 (1991), incorporated herein by reference. Various antibody-containing phage display libraries have been described and may be readily prepared by one skilled in the art. Libraries may contain a diversity of human antibody sequences, such as human Fab, Fv, and scFv fragments, that may be screened against an appropriate target. After phage selection, the antibody-coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)₂ fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. The V_{H} and V_{L} domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or fragment thereof which is capable of binding to a predetermined antigen and which comprises a framework region having substantially the amino acid sequence of a human immunoglobulin, and a CDR having substantially the amino acid sequence of a non-human immunoglobulin. A humanized antibody comprises substantially all of at least one, and typically two, variable domains (Fab, Fab′, F(ab′)2, Fabc, Fv) in which all or substantially all of the CDR regions correspond to those of a non-human immunoglobulin (i.e., donor antibody) and all or substantially all of the framework regions are those of a human immunoglobulin consensus sequence. Humanized antibodies can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu 3 S, Gene 150(2):409-10 (1994), and Pedersen et al., 3. Mol. Biol. 235(3):959-73 (1994). See also U.S. Patent Pub. No. US 2005/0042664 A1 (Feb. 24, 2005), which is incorporated by reference herein in its entirety.

Single domain antibodies, for example, antibodies lacking the light chains, can be produced by methods well-known in the art. See Riechmann et al., 1999, J. Immunol. 231:25-38; Nuttall et al, 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that bind to a NAV protein antigen can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438). Also provided herein is a B-cell (e.g., an immortalised B-cell) or a hybridoma that produces an anti-NAV protein antibody or fragment described herein.

The antibodies as disclosed herein include antibodies that are chemically modified, i.e., by the covalent attachment of any type of molecule to the antibody to provide a fusion protein or antibody conjugate. For example, the antibody derivatives include antibodies that have been chemically modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formulation, metabolic synthesis of tunicamycin, etc. Additionally, the antibody may contain one or more non-classical amino acids.

There are also provided antibodies that bind to a NAV protein antigen which comprise a framework region known to those of skill in the art (e.g., a human or non-human fragment). The framework region may, for example, be naturally occurring or consensus framework regions. Most preferably, the framework region of an antibody as disclosed herein is human (see, e.g., Chothia et al., 1998, J. Mol. Biol. 278:457-479 for a listing of human framework regions, which is incorporated by reference herein in its entirety). See also Kabat et al. (1991) Sequences of Proteins of Immunological Interest (U.S. Department of Health and Human Services, Washington, D.C.) 5th ed. In some embodiments, human antibodies are produced. Human antibodies and/or fully human antibodies can be produced using any method known in the art, including the Examples provided herein and discussed in further detail below. Exemplary methods include immunization with a NAV protein antigen (any NAV protein polypeptide capable of eliciting an immune response, and optionally conjugated to a carrier, see the Examples below for exemplary immunisation schedules) of transgenic animals (e.g., mice) that are capable of producing a repertoire of human antibodies in the absence of endogenous immunoglobulin production; see, e.g., Jakobovits et al., (1993) Proc. Natl. Acad. Sci., 90:2551; Jakobovits et al., (1993) Nature, 362:255 258 (1993); Bruggermann et al., (1993) Year in Immunol., 7:33. Other methods of producing fully human anti-NAV protein antibodies can be found in the Examples provided herein. In addition, companies such as Abgenix, Inc. (Freemont, Calif.) and Genpharm (San Jose, Calif.) can be engaged to provide human antibodies directed against a selected antigen using technology similar to that described above.

In an example, the binding site(s) of the antibody are selected from a plurality (e.g., library) of binding sites. For example, the plurality of binding sites comprises or consists of a plurality of 4-chain antibodies or fragments thereof, e.g., dAbs, Fabs or scFvs. Suitable methods for producing pluralities of binding sites for screening include phage display (producing a phage display library of antibody binding sites), ribosome display (producing a ribosome display library of antibody binding sites), yeast display (producing a yeast display library of antibody binding sites), or immunisation of a non-human vertebrate (e.g., a rodent, e.g., a mouse or rat, such as a Velocimouse™, Kymouse™, Xenomouse™, Aliva Mouse™, HuMab Mouse™, Omnimouse™, Omnirat™ or MeMo Mouse™) with NAV protein or a NAV protein polypeptide epitope and isolation of a repertoire of antibody-producing cells (e.g., a B-cell, plasma cell or plasmablast repertoire) and/or a repertoire of isolated antibodies, fragments or binding sites.

Because of the complex nature of the sodium channel architecture, immunisation of an animal with a linear peptide may not always generate antibodies which are capable of binding (and therefore blocking ion transport) in the native, membrane bound form. In one embodiment, the animal (e.g. mouse, such as a Kymouse™) is immunised with a linear NAV polypeptide of interest (such as a polypeptide as in any of SEQ ID No: 4, 6, 8, 10, 12, 14, 16, 18, 22, 24, 26, 28, 30, 32, 34, 36, 40, 42, 44, 46, 48, 50, 52, 54, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328, in particular SEQ ID No: 6, 10, 14, 18, 24, 28, 32, 36, 42, 46, 50, 54). However, in order to increase the likelihood of isolating antibodies which are able to bind and block native NAV proteins, there is provided an immunisation procedure to generate an antibody against a NAV protein of interest as set out in any one of sentences 363 to 397:

Sentence 363. A method of generating an antibody against a NAV protein of interest comprising the steps of:
a. Immunising a non-human mammal with MEF or HEK (e.g. HEK) cells which express said NAV protein of interest on its surface; and
b. Immunising said non-human mammal with one or two fragment(s) of said NAV protein of interest.

Sentence 364. The method according to sentence 363 further comprising the steps of:
c. Immunising said non-human mammal with MEF or HEK (e.g. HEK) cells which express said NAV protein of interest on its surface; and
d. Immunising said non-human mammal with said one or two fragment(s) of the NAV protein of interest.

In the immunisation procedures as disclosed herein, when two fragments of the NAV protein of interest are employed, in a first embodiment, the two fragments have the same linear amino acid sequence of the NAV protein of interest (e.g. from one of the external loop sequences (i.e. E1, E2 or E3) of any of the four domains of the NAV proteins), but one fragment has been modified to include a cysteine at the C terminus and the other has been modified to include a cysteine at the N terminus, to each of which a carrier (e.g. KLH) is attached. In another embodiment, one of the two fragments of the NAV protein of interest comprises a linear amino acid sequence of the NAV protein of interest (e.g. from one of the external loop sequences (i.e. E1, E2 or E3 of any of the four domains of the NAV proteins), which been modified to include a cysteine at either the C terminus or the N terminus, to which a carrier (e.g. KLH) is attached and the other fragment comprises a cyclic amino acid sequence of the NAV protein of interest (e.g. from one of the external loop sequences (i.e. E1, E2 or E3) of any of the four domains of the NAV proteins, such as from the same loop as the linear fragment). In another embodiment, two linear conjugated fragments are used (as described above), but each fragment has an amino acid sequences which corresponds to a different external loop of the NAV protein of interest (e.g. the first fragment comprises amino acids from the D1E1 loop, and the second fragment comprises amino acids from the D1E2 loop). In another embodiment only one fragment (either a linear fragment conjugated to a carrier at either the C or N terminus, or a cyclic fragment as described above) is employed in the method.

Sentence 365. The method according to sentence 364 further comprising the step of either:
e. Immunising said non-human mammal with MEF or HEK (e.g. HEK) cells which express said NAV protein of interest on its surface; or
f. Immunising said non-human mammal with said one or two fragment(s) of the NAV protein of interest.

In one embodiment, the cells are HEK cells expressing the NAV protein of interest (e.g. hNAV1.7, hNAV1.8 and/or hNAV1.9)

Sentence 366. The method of sentence 365, further comprising the step of:
g. Immunising said non-human mammal with said one or two fragment(s) of the NAV protein of interest.

Sentence 367. The method according to sentence 364, wherein the order of the steps is a. then b. then c. then d.

Sentence 368. The method according to sentence 364, wherein the order of the steps is b. then a. then d. then c.

Sentence 369. The method according to sentence 365, wherein the order of steps is b. then a. then
d. then c. then f.

Sentence 370. The method according to sentence 366, wherein the order of the steps is b. then a. then d. then c. then f. then g.

Sentence 371. The method according to any one of sentences 364 to 370, which comprises an additional step of priming said non-human mammal by immunising with MEF or HEK cells which express said NAV protein of interest on its surface before the first step of any one of sentences 364 to 370.

Sentence 372. The method according to sentence 371, wherein from 1×10⁶ to 3×10⁶ (e.g. about 2×10⁶), or from 0.5×10⁷ to 3×10⁷ (e.g. about 1×10⁷, or about 2×10⁷) of MEF or HEK (e.g. HEK) cells are used in the priming dose.

Sentence 373. The method according to sentence 371 or sentence 372, wherein the HEK or MEK (e.g. HEK) cells are adjuvanted with (S6322), optionally at a concentration of 10-50%% (v/v).

Sentence 374. The method according to any one of sentences 364 to 370, which comprises an additional step of priming said non-human mammal by immunising said non-human mammal with said one or two fragment(s) of said NAV protein of interest before the first step of any one of sentences 364 to 370.

Sentence 375. The method according to sentence 374, wherein said one or two fragment(s) of said NAV protein of interest is/are adjuvanted with a combination of CFA (optionally at a concentration of 50% (v/v)), CpG (optionally at a concentration of 0.1 mg/ml of ODN 1826) and Alum (optionally at a concentration of 25% (v/v) of Alhydrogel 2%).

Sentence 376. The method according to any one of sentences 364 to 375, wherein the NAV protein of interest is NAV1.7, NAV1.8 or NAV1.9.

Sentence 377. The method according to any one of sentences 364 to 376, wherein the one or two fragment(s) of said NAV protein of interest is one or two loop region(s) on said NAV protein of interest or a fragment thereof.

Sentence 378. The method according to sentence 377, wherein the one or two loop region(s) are selected from the D1E1, the D1E2, the D1E3, the D2E1, the D2E2, the D2E3, the D3E1, the D3E2, the D3E3, the D4E1, the D4E2 or the D4E3 loop regions on the NAV protein of interest, or a fragment thereof.

Sentence 379. The method according to sentence 377, where in one of the loop regions is selected from the D1E2, the D2E2, the D3E2 or the D4E2 loop regions of the NAV protein of interest, or a fragment thereof.

Sentence 380. The method according to sentence 377, wherein one or both of the fragments of said NAV protein of interest comprise a sequence selected from SEQ ID No: 4, 6, 8, 10, 12, 14, 16, 18, 22, 24, 26, 28, 30, 32, 34, 36, 40, 42, 44, 46, 48, 50, 52, 54, 86, 90, 96, 100, 104, 108, 114, 158, 162, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328 (in particular SEQ ID No: 6, 10, 14, 18, 24, 28, 32, 36, 42, 46, 50, 54), optionally wherein the fragment(s) are conjugated to a carrier, such as KLH or ova, e.g. KLH.

When conjugated, the fragment(s) of the NAV protein of interest are modified to include a cysteine residue at either the C terminus or the N terminus, which allows the carrier to be chemically conjugated to the fragment.

Sentence 381. The method according to any one of sentences 364 to 379, wherein the immunisations are carried out with a single fragment of said NAV protein of interest.

Sentence 382. The method according to sentence 380 wherein:
a. the amount of said fragment of said NAV protein of interest is 20 μg in the first immunisation with said fragment (step b); and
b. the amount of said fragment of said NAV protein of interest is 5 μg in the second immunisation with said fragment (step d); and optionally
c. the amount of said fragment of said NAV protein of interest is 1 μg in the third immunisation with said fragment (step f); and optionally
d. the amount of said fragment of said NAV protein of interest is 1 μg in the fourth immunisation with said fragment (step g).

Sentence 383. The method according to any one of sentences 364 to 379, wherein the immunisations are carried out with a two different fragments of said NAV protein of interest.

Sentence 384. The method according to sentence 382 wherein:
a. the amount of said two fragments of said NAV protein of interest is either 10 μg of each or 5 μg of each in the first immunisation with said fragments (step b); and
b. the amount of said two fragments of said NAV protein of interest is 3 μg of each in the second immunisation with said fragments (step d); and optionally
c. the amount of said fragment of said NAV protein of interest is either 3 μg of each or 0.5 μg of each in the third immunisation with said fragments (step f); and optionally
d. the amount of said fragment of said NAV protein of interest is either 1 μg of each or 0.5 μg of each in the fourth immunisation with said fragments (step g).

When two fragments are employed, the total amount of polypeptide used in the immunisation is, in one embodiment, the same as the amount which would have been used if a single fragment were employed. As known to a person skilled in the art, with each immunisation, it is usual for the amount of polypeptide employed to decrease.

Sentence 385. The method according to any one of sentences 364 to 384, wherein the HEK or MEK (e.g. HEK) cells used in the immunisations are administered intraperitoneally or intravenously (e.g. intraperitoneally), optionally in an amount of from 1×10⁶ to 3×10⁶ cells (e.g. about 2×10⁶) or from 4×10⁶ to 6×10⁶ (e.g. about 5×10⁶), optionally adjuvanted with Sigma adjuvant (e.g. S6322).

Sentence 386. The method according to sentence 385, wherein the HEK or MEK (e.g. HEK) cells are unadjuvanted in step a., step c. or step e.

When used in the priming dose, the cells are usually adjuvanted, whereas, in one embodiment, the booster doses comprise unadjuvanted cells. When the last booster is a booster of cells, then, in another embodiment, no adjuvant is employed. The final boosting dose is usually administered intravenously.

Sentence 387. The method according to any one of sentences 364 to 386, wherein the one or two fragment(s) of said NAV protein of interest is/are adjuvanted in one, two or all of step b., step d., step f. and step g. with (i) a combination of Sigma (optionally at a concentration of 2% (v/v)), CpG (optionally at a concentration of 0.1 mg/ml of oligodeoxynucleotide ODN 1826) and Alum (optionally at a concentration of 25% hydrogel (v/v)); or a combination of IFA (optionally at a concentration of 50% (v/v)), CpG (optionally at a concentration of 0.1 mg/ml of ODN 1826) and Alum (optionally at a concentration of 25% Alhydrogel 2% (v/v).

Sentence 388. The method according to any one of sentences 364 to 387, wherein the one or two fragment(s) of said NAV protein of interest is/are administered intraperitoneally or intravenously (e.g. intraperitoneally).

In some embodiments, all doses, whether of cells or of fragment(s), except for the final dose, are administered intraperitoneally. The final dose may be administered intravenously.

Sentence 389. The method according to any one of sentences 364 to 388, wherein the final step of immunisation is carried out intravenously without any adjuvant.

Sentence 390. The method according to any one of sentences 364 to 389, wherein the non-human animal has, before each immunisation, been dosed with a compound which stabilises said NAV protein of interest in an open, closed or activated conformation, in an amount sufficient to stabilise said NAV protein of interest in said open conformation, optionally wherein the compound is selected from tetrodotoxin (TTX), saxitoxin (STX), hanatoxin, centipede toxin, μ-SLPTX-Ssm6a, Protoxin-I (ProTx-I), Protoxin-II (ProTx-II), Huwentoxin-IV (HwTx-IV) and a conotoxin (such as μ-GIIIA, μ-GIIIB, μ-GIIIC, μ-PIIIA, μ-TIIIA, μ-SmIIIA, μ-KIIIA, μ-SIIIA, μ-CoIIIA, μ-CoIIIB, μ-CIIIA, μ-MIIIA, μO-MrVIA, μO-MrVIB, δ-TxVIA, δ-TxVIB, δGmVIA, δ-PVIA, δ-NgVIA, δ-EVIA and δ-SVIE, in particular μ-KIIIA).

By stabilising the NAV protein of interest in a particular conformation, the likelihood of isolating an antibody which binds to the native NAV protein of interest when in that same conformation is increased.

Sentence 391. The method according to any one of sentences 364 to 390, wherein the non-human mammal is mouse or rat, e.g., a Velocimouse™, Kymouse™, Xenomouse™, Aliva Mouse™, HuMab Mouse™, Omnimouse™, Omnirat™ or MeMo Mouse™ (e.g. KyMouse™).

Sentence 392. The method according to any one of sentences 364 to 390, which further comprises the step of isolating an antibody which specifically binds the NAV protein of interest.

Sentence 393. The method according to sentence 392, wherein the isolated antibody is screened in one or more in vitro assays for activity against the NAV protein of interest.

The assays employed may be any of the assays described hereinabove, including analysis of affinity, potency and/or efficacy (e.g. Kd, IC₅₀ and/or degree of maximum inhibition).

Sentence 394. The method according to sentence 393, wherein the assay is a standard whole cell patch clamp assay, optionally as defined in any one of sentences 316 to 319.

Sentence 395. The method according to sentence 393 or sentence 296, wherein the assay comprises screening for binding against said NAV protein of interest, wherein said NAV protein of interest has been stabilised in the open, closed or activated state, optionally by binding of a compound selected from tetrodotoxin (TTX), saxitoxin (STX), hanatoxin, centipede toxin, μ-SLPTX-Ssm6a, Protoxin-I (ProTx-I), Protoxin-II (ProTx-II), Huwentoxin-IV (HwTx-IV) and a conotoxin (such as μ-GIIIA, μ-GIIIB, μ-GIIIC, μ-PIIIA, μ-TIIIA, μ-SmIIIA, μ-KIIIA, μ-SIIIA, μ-CoIIIA, μ-CoIIIB, μ-CIIIA, μ-MIIIA, μO-MrVIA, μO-MrVIB, δ-TxVIA, δ-TxVIB, δGmVIA, δ-NgVIA, δ-EVIA and δ-SVIE, in particular μ-KIIIA).

Sentence 396. The method according to any one of sentences 392 to 395, which further comprises humanising the isolated antibody.

Sentence 397. The method according to sentence 396, which further comprises stably expressing said isolated, humanised antibody.

Various adjuvants may be used in any of the above sentences to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

In any of the sentences above (363 to 397), the doses (i.e. priming and boosters) are spaced apart by 10 to 37 days, e.g. 12-16 days, or 19-23 days or 22-26 days. Each of the dose volumes may be 50 μl, 100 μl or 200 μl, e.g. 100 μl.

Additionally, a RIMMS (repetitive immunization multiple sites) technique can be used to immunize an animal (Kilptrack et al., 1997 Hybridoma 16:381-9, incorporated by reference in its entirety). The hybridoma clones are then assayed by methods known in the art for cells that secrete antibodies capable of binding a NAV protein antigen. Ascites fluid, which generally contains high levels of antibodies, can be generated by immunizing mice with positive hybridoma clones.

Also provided herein is an antibody obtainable by the method as defined in any one of sentences 364 to 397.

Methods for producing and screening for specific antibodies using hybridoma technology are routine and well known in the art. Briefly, mice can be immunized with a NAV protein antigen and once an immune response is detected, e.g., antibodies specific for NAV protein antigen are detected in the mouse serum, the mouse spleen is harvested and splenocytes isolated. The splenocytes are then fused by well-known techniques to any suitable myeloma cells, for example cells from cell line SP20 available from the ATCC. Hybridomas are selected and cloned by limited dilution.

Accordingly, there are provided methods of generating antibodies by culturing a hybridoma cell secreting an anti-NAV protein antibody as disclosed herein, wherein in one embodiment, the hybridoma is generated by fusing splenocytes isolated from a mouse immunized with a NAV protein antigen with myeloma cells and then screening the hybridomas resulting from the fusion for hybridoma clones that secrete an antibody able to bind to a NAV protein antigen.

In another aspect, provided herein are isolated nucleic acids encoding antibodies that bind to a NAV protein of interest (e.g., a cell surface-expressed NAV protein), a NAV protein polypeptide, or a NAV protein epitope. In certain embodiments, the nucleic acid encodes a V_{H} chain or a V_{L} chain. In certain other embodiments, the nucleic acid encodes a heavy chain or a light chain. In other embodiment, the nucleic acid encodes a V_{H} chain and a V_{L} chain of an anti-NAV protein antibody as described herein (in any of the embodiments or combination of embodiments). In another embodiment, there is provided a nucleic acid that encodes a CHRH1, CDRH2, CDRH3, CDRL1, CDRL2 and/or CDRL3 of an anti-NAV antibody as disclosed herein. In another embodiment, there is provided a nucleic acid that encodes CHRH1, CDRH2 and/or CDRH3 of an anti-NAV antibody as disclosed herein. In another embodiment, there is provided a nucleic acid that encodes CDRL1, CDRL2 and/or CDRL3 of an anti-NAV antibody as disclosed herein. In another embodiment, there is provided a nucleic acid that encodes either CDRH3 or CDRL3 of an anti-NAV antibody as disclosed herein.

In another aspect, provided herein are vectors (such as HEK293 vectors or CHO vectors) and host cells (such as HEK cells, CHO cells, NSO cells or COS cells, e.g. HEK293 cells, or COS7 cells) comprising nucleic acids encoding antibodies or fragments of the invention. In one embodiment, the antibodies as described herein are expressed from a CHO cell.

### Antibody Conjugates and Fusion Proteins

The following discussion on conjugates and fusion proteins also applies to fragments so that disclosure mentioning antibodies that bind to a NAV protein antigen (as disclosed herein in any embodiment, or combination of embodiments) can also apply mutatis mutandis to fragments of the invention.

There are provided fusion proteins comprising an antibody provided herein that binds to a NAV protein antigen and a heterologous polypeptide or chemical entity. In some embodiments, the heterologous polypeptide to which the antibody is fused is useful for targeting the antibody to cells having cell surface-expressed NAV protein.

In some embodiments, anti-NAV antibodies as disclosed herein are conjugated or recombinantly fused to a diagnostic, detectable or therapeutic agent or any other molecule. The conjugated or recombinantly fused antibodies can be useful, e.g., for monitoring or prognosing the onset, development, progression and/or severity of a NAV protein-mediated disease as part of a clinical testing procedure, such as determining the efficacy of a particular therapy.

Such diagnosis and detection can accomplished, for example, by coupling the anti-NAV antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I), carbon (¹⁴C), sulfur (³⁵S), tritium (³H), indium (¹¹⁵In, ¹¹³In, ¹¹²In, and ¹¹¹In), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe) and fluorine (¹⁸F); and positron emitting metals using various positron emission tomographies, and non-radioactive paramagnetic metal ions.

There are provided uses of the anti-NAV antibodies as disclosed herein conjugated or recombinantly fused to a therapeutic moiety (or one or more therapeutic moieties). The antibody may be conjugated or recombinantly fused to a therapeutic moiety, such as an opioid analgesic (e.g. morphine, diamorphine, codeine, dihydrocodeine, fentanyl, oxycodone, buprenorphine, dextropropoxyphene, tramadol, meptazinol, pethidine or pantazocine), paracetamol, a non-steroidal anti-inflammatory (e.g. aspirin, ibuprofen, ketoprofen, naproxen, indomethacin, diclofenac, celecoxib, ketorolac, mefenamic acid, meloxicam, piroxicam, nabumetone, parecoxib, sulindac or tenoxicam), a local anaesthetic (e.g. bupivacaine, lignocaine), a 5HT1 agonist (e.g. sumptriptan or naratriptan), an anti-epileptic/antidepressant (e.g. carbamazepine, gabapentin, pregabalin or duloxetine), an anxiolytic/muscle relaxant (e.g. diazepam, tizanidine or cyclobenzaprine), ziconitide, botulinum toxin, tetrahydrocannabinol, cannabidiol, capsaicin, an anti-NGF drug, and anti-TrkA drug, an anti-CGRP drug, p75NTR-Fc, a COX-1 antagonist, a COX-2 antagonist, a TRPV1 antagonist, a TRPV3 agonist, a voltage-gated sodium channel blocker or a FAAH inhibitor

There are provided anti-NAV antibodies as disclosed herein recombinantly fused or chemically conjugated (covalent or non-covalent conjugations) to a heterologous protein or polypeptide to generate fusion proteins. In particular, there are provided fusion proteins comprising an antigen-binding fragment of an anti-NAV protein antibody as disclosed herein (e.g., a Fab fragment, Fd fragment, Fv fragment, F(ab)₂ fragment, a V_{H} domain, a V_{H} CDR, a V_{L} domain or a V_{L} CDR) and a heterologous protein or polypeptide.

Moreover, anti-NAV antibodies as disclosed herein can be fused to marker sequences, such as a peptide to facilitate purification. In preferred embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et at, Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992, which are incorporated herein by reference in their entireties.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of anti-NAV protein antibodies as disclosed herein (e.g., antibodies with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313 (each of these patents and publications are hereby incorporated by reference in its entirety). Antibodies, or the encoded antibodies, may be altered by being subjected to random mutagenesis by error-prone PCR, random nucleotide insertion or other methods prior to recombination. A polynucleotide encoding an anti-NAV protein antibodies as disclosed herein may be recombined with one or more components, motifs, sections, parts, domains, fragments, etc. of one or more heterologous molecules.

Anti-NAV protein antibodies as disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Compositions

The following discussion on pharmaceutical compositions comprising antibodies as disclosed herein also applies mutatis mutandis to fragments of the invention. In one embodiment, there is provided a pharmaceutical composition comprising an anti-NAV protein antibody as described herein in any embodiment, or combination of embodiments, and a diluent, excipient or carrier.

In an example, an antibody as disclosed herein is contained in a medical container, e.g., a vial, syringe, IV container or an injection device (such as an intraocular or intravitreal injection device). In an example, the antibody is in vitro, for example, in a sterile container.

In a specific embodiment, a composition comprises one, two or more anti-NAV protein antibody(s). In another embodiment, a composition comprises one, two or more anti-NAV protein antibody(s) and a prophylactic or therapeutic agent other than an anti-NAV protein antibody as disclosed herein, such as an anti-nociceptive drug. Preferably, the agents are known to be useful for or have been or are currently used for the prevention, management, treatment and/or amelioration of a NAV protein-mediated disease, such as pain management. In one embodiment, the anti-nociceptive drug is selected from an opioid analgesic (e.g. morphine, diamorphine, codeine, dihydrocodeine, fentanyl, oxycodone, buprenorphine, dextropropoxyphene, tramadol, meptazinol, pethidine or pantazocine), paracetamol, a non-steroidal anti-inflammatory (e.g. aspirin, ibuprofen, ketoprofen, naproxen, indomethacin, diclofenac, celecoxib, ketorolac, mefenamic acid, meloxicam, piroxicam, nabumetone, parecoxib, sulindac or tenoxicam), a local anaesthetic (e.g. bupivacaine, lignocaine), a 5HT1 agonist (e.g. sumptriptan or naratriptan), an anti-epileptic/antidepressant (e.g. carbamazepine, gabapentin, pregabalin or duloxetine), an anxiolytic/muscle relaxant (e.g. diazepam, tizanidine or cyclobenzaprine), ziconitide, botulinum toxin, tetrahydrocannabinol, cannabidiol, capsaicin, an anti-NGF drug, and anti-TrkA drug, an anti-CGRP drug, p75NTR-Fc, a COX-1 antagonist, a COX-2 antagonist, a TRPV1 antagonist, a TRPV3 agonist, a voltage-gated sodium channel blocker or a FAAH inhibitor.

In addition to prophylactic or therapeutic agents (e.g. anti-nociceptive drugs as described above), the compositions may also comprise a carrier.

In a specific embodiment, the term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

The pharmaceutical compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences (1990) Mack Publishing Co., Easton, Pa. Such compositions will contain a prophylactically or therapeutically effective amount of the anti-NAV protein antibody, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In another embodiment, a prophylactic or therapeutic agent, or a composition as described herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition as described herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et at, 1980, Surgery 88:507; Saudek et al., 1989, N. Engl. J. Med. 321:574). In another embodiment, polymeric materials can be used to achieve controlled or sustained release of a prophylactic or therapeutic agent (e.g., an anti-NAV protein antibody as described herein) or a composition as described herein. In a preferred embodiment, the polymer used in a sustained release formulation is inert, free of leachable impurities, stable on storage, sterile, and biodegradable.

In a specific embodiment, where the composition is a nucleic acid encoding a prophylactic or therapeutic agent (e.g., an anti-NAV protein antibody), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

In another embodiment, there is provided a pharmaceutical composition for use in treating and/or preventing a NAV-mediated condition or disease, the composition comprising an antibody or fragment as described herein in any embodiment or combination of embodiments (and optionally an anti-nociceptive drug as described hereinabove) optionally in combination with a label or instructions for use to treat and/or prevent said disease or condition in a human; optionally wherein the label or instructions comprise a marketing authorisation number (e.g., an FDA or EMA authorisation number).

### Methods of Administration, Treatment and Dosing

There are provided compositions comprising one or more anti-NAV protein antibody(s) as disclosed herein in any embodiment, or combination or embodiments, for use in the prevention, management, treatment and/or amelioration of a NAV protein-mediated disease (or symptom thereof). Discussion in respect of antibodies also applies mutatis mutandis to fragments of the invention.

In certain embodiments, the route of administration for a dose of an anti-NAV protein antibody as disclosed herein to a patient is intranasal, intramuscular, intravenous, or a combination thereof, but other routes described herein are also acceptable. Each dose may or may not be administered by an identical route of administration. In some embodiments, an anti-NAV protein antibody as disclosed herein may be administered via multiple routes of administration simultaneously or subsequently to other doses of the same or a different anti-NAV protein antibody as disclosed herein.

Various delivery systems are known and can be used to administer a prophylactic or therapeutic agent (e.g., an antibody as disclosed herein), including, but not limited to, encapsulation in liposomes, microparticles, microcapsules, recombinant cells capable of expressing the antibody, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an antibody as disclosed herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antibody as disclosed herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents, such as an anti-nociceptive drug. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO92/19244, WO97/32572, WO97/44013, WO98/31346, and WO99/66903, each of which is incorporated herein by reference their entirety.

In a specific embodiment, it may be desirable to administer a prophylactic or therapeutic agent, or a pharmaceutical composition as described herein locally to the area in need of treatment. This may be achieved by, for example, local infusion, by topical administration (e.g., by intranasal spray), by injection, or by means of an implant, said implant being of a porous, non-porous, or gelatinous material, including membranes, such as sialastic membranes, or fibres. Preferably, when administering an anti-NAV protein antibody, care must be taken to use materials to which the antibody does not absorb.

In an aspect, there is provided a method of treating and/or preventing and/or reducing the risk of a NAV-mediated disease or condition in a subject (e.g. a human subject) by administering to said subject a therapeutically effective amount of an antibody that binds to a NAV protein of interest (e.g. a hNAV1.7, hNAV1.8 and/or hNAV1.9 protein, optionally as a full-length NAV protein expressed on a cell surface), wherein the disease or condition is treated and/or prevented, and/or wherein the risk of the disease or condition is reduced by the antibody. In an example, the method comprises decreasing or inhibiting a NAV protein biological activity, such as reducing ion flux, in the subject.

In another aspect, there is provided an anti-NAV protein antibody (as described in any of the embodiments, or combination of embodiments herein, such as an anti-hNAV1.7, anti-hNAV1.8 and/or anti-hNAV1.9 antibody) for use in therapy. In an alternative embodiment, there is provided an anti-NAV protein antibody (as described in any of the embodiments, or combination of embodiments herein, such as an anti-hNAV1.7, anti-hNAV1.8 and/or anti-hNAV1.9 antibody) for use in the treatment (e.g. the treatment) and/or prevention of a NAV-mediated disease or condition.

In an alternative embodiment, there use on is provided the use of an anti-NAV protein antibody (as described in any of the embodiments, or combination of embodiments herein, such as an anti-hNAV1.7, anti-hNAV1.8 and/or anti-hNAV1.9 antibody) in the manufacture of a medicament for administration to a human, for treating and/or preventing (e.g. treating) a NAV-mediated disease or condition in the human.

In another embodiment, there is provided a pharmaceutical composition for use in treating, preventing and/or reducing the risk of a NAV-mediated condition or disease, the composition comprising an antibody as described herein in any embodiment or combination of embodiments, and a diluent, excipient or carrier; and optionally further comprising an anti-nociceptive drug.

The NAV-mediated disease or condition may be a NAV1.7-mediated disease or condition, a NAV1.8-mediated disease or condition and/or a NAV1.9-mediated disease or condition.

The NAV-mediates diseases or conditions may be selected from:

a. Neuropathic/neurogenic pain (for example arising from painful diabetic neuropathy (PDN), post-herpetic neuropathy (PHN), central neuropathy, peripheral neuropathy, trigeminal neuralgia (TN), anaesthesia dolorosa, spinal cord injuries, multiple sclerosis, phantom limb pain, hyperalgesia, hyperpathia, paresthesia, psychogenic pain, post-stroke pain and HIV-associated pain, back pain, chronic back pain, osteoarthritis, cancer, breakthrough pain, erythromelalgia [e.g. primary erythromelalgia], paroxysmal extreme pain disorder, nerve compression and/or entrapment [such as carpal tunnel syndrome, tarsal tunnel syndrome, ulnar nerve entrapment, compression radiculopathy, radicular low back pain, spinal root lesions, spinal root compression, lumbar spinal stenosis, sciatic nerve compression, intercostal neuralgia], neuritis, pain from chemotherapy, congenital defect/channelopathy [e.g. channelopathy-associated insensitivity to pain and congenital insensitivity to pain], chronic alcoholism [alcoholic polyneuropathy]);

b. inflammation (such as osteoarthritis, chronic back pain, rheumatoid arthritis, cancer, breakthrough pain, burns, encephalitis, bone fracture, neuritis, autoimmune diseases, postoperative pain, dental pain, bacterial infection, radiotherapy, gout and irritable bowel syndrome);

c. pain from trauma (such as from lacerations, incisions, burns, foreign bodies or bullet and/or shrapnel injuries, spinal cord injury, brachial plexus avulsion, nerve crush and/or entrapment (such as carpal tunnel syndrome, tarsal tunnel syndrome, ulnar nerve entrapment, compression radiculopathy, radicular low back pain, spinal root lesions, spinal root compression, lumbar spinal stenosis, sciatic nerve compression, intercostal neuralgia), nerve transection, post-operative pain, dental pain and toxic exposure);

d. pain from infection (such as post-herpetic neuropathy (PHN), HIV-associated pain small pox infection, encephalitis, herpes infection, and bacterial infection);

e. pain from malignancy (such as cancer pain, breakthrough pain, and nerve compression pain);

f. visceral pain (such as renal/ureteral colic, irritable bowel syndrome, angina/cardiac pain, cardiac arrhythmia, period pain, interstitial cystitis, rectal pain, pain associated with diarrhoea, appendicitis, cholecystitis and pancreatitis);

g. metabolic/chronic disease (such as multiple sclerosis, cancer pain, breakthrough pain, gout, peripheral diabetic neuropathy, chronic alcoholism [alcoholic polyneuropathy], uremia, hypothyroidism and vitamin deficiency);

h. headache pain (such as tension headache, migraine and cluster headaches);

i. idiopathic pain (such as trigeminal neuralgia, complex regional pain syndromes [e.g. complex regional pain syndrome I and complex regional pain syndrome II], allodynia and fibromyalgia);

j. respiratory pain (such as pain associated with asthma, airway hyper-reactivity in asthma, chronic cough, e.g. in asthma and/or chronic obstructive pulmonary disorder); or

k. other pain (such as pain associated with hormonal therapy, diabetes, hypothyroidism, epilepsy, ataxia, periodic paralysis, acute itch and chronic itch).

In another embodiment, the NAV-mediated disease or condition is selected from painful diabetic neuropathy, post-herpetic neuropathy, trigeminal neuralgia, osteoarthritis, chronic back pain, nerve compression pain (e.g. sciatic nerve compression) or cancer pain. In a further embodiment, the NAV-mediated disease or condition is selected from migraine, post-operative pain and fibromyalgia. In a further embodiment, the NAV-mediated disease or condition is a channelopathy. In another embodiment, the NAV-mediated disease or condition is painful diabetic neuropathy. In another embodiment, the NAV-mediated disease or condition is post-herpetic neuropathy. In another embodiment, the NAV-mediated disease or condition is trigeminal neuralgia. In another embodiment, the NAV-mediated disease or condition is osteoarthritis. In another embodiment, the NAV-mediated disease or condition is chronic back pain. In another embodiment, the NAV-mediated disease or condition is nerve compression pain (e.g. sciatic nerve compression). In another embodiment, the NAV-mediated disease or condition is cancer pain. In another embodiment, the NAV-mediated disease or condition is post-operative pain. In another embodiment, the NAV-mediated disease or condition is migraine. In another embodiment, the NAV-mediated disease or condition is fibromyalgia.

In other embodiments, there are provided methods of targeting certain NAV proteins, as set out in sentences 351 to 362 below:

Sentence 351. A method of selectively targeting NAV1.7 in a human, comprising administering to said human patient an antibody as defined in any embodiment or combination of embodiments described herein, including in sentence 398 wherein NAV1.7 is targeted.

Sentence 352. The method according to sentence 351, wherein the method targets NAV1.7 over one, more (e.g. 2, 3, 4, 5) or all of NAV1.1, NAV1.2, NAV1.3, NAV1.4, NAV1.5 and NAV1.6.

Sentence 353. The method according to sentence 351, wherein the method targets NAV1.7 over NAV1.8 and/or NAV 1.9.

Sentence 354. The method according to any one of sentences 351 to 353, wherein the method treats or reduces the risk of a NAV1.7-mediated disease or condition in said human.

Sentence 355. A method of selectively targeting NAV1.8 in a human, comprising administering to said human patient an antibody as defined in any embodiment or combination of embodiments described herein, including in sentence 398, wherein NAV1.8 is targeted.

Sentence 356. The method according to sentence 355, wherein the method targets NAV1.8 over one, more (e.g. 2, 3, 4, 5) or all of NAV1.1, NAV1.2, NAV1.3, NAV1.4, NAV1.5 and NAV1.6.

Sentence 357. The method according to sentence 355, wherein the method targets NAV1.8 over NAV1.7 and/or NAV 1.9.

Sentence 358. The method according to any one of sentences 355 to 357, wherein the method treats or reduces the risk of a NAV1.8-mediated disease or condition in said human.

Sentence 359. A method of selectively targeting NAV1.9 in a human, comprising administering to said human patient an antibody as defined in any embodiment or combination of embodiments described herein, including in sentence 398, wherein NAV1.9 is targeted.

Sentence 360. The method according to sentence 359, wherein the method targets NAV1.9 over one, more (e.g. 2, 3, 4, 5) or all of NAV1.1, NAV1.2, NAV1.3, NAV1.4, NAV1.5 and NAV1.6.

Sentence 361. The method according to sentence 359, wherein the method targets NAV1.9 over NAV1.7 and/or NAV 1.8.

Sentence 362. The method according to any one of sentences 359 to 361, wherein the method treats or reduces the risk of a NAV1.9-mediated disease or condition in said human.

In any of sentences 352, 356 or 360, the method may target NAV1.7, NAV1.8 or NAV1.9 over at least NAV1.4 and NAV1.5. In a further embodiment, the method may target NAV1.7, NAV1.8 or NAV1.9 over at least NAV1.4 and NAV1.6. In a further embodiment, the method may target NAV1.7, NAV1.8 or NAV1.9 over at least NAV1.5 and NAV1.6. In a further embodiment, the method may target NAV1.7, NAV1.8 or NAV1.9 over at least NAV1.4, NAV1.5 and NAV1.6.

In any of sentences 352, 356 or 360, the method may target NAV1.7, NAV1.8 or NAV1.9 over at least three other NAV proteins. Thus, in a further embodiment, the method may target NAV1.7, NAV1.8 or NAV1.9 over at least NAV1.1, NAV1.2 and NAV1.3. In a further embodiment, the method may target NAV1.7, NAV1.8 or NAV1.9 over at least NAV1.1, NAV1.2 and NAV1.4. In a further embodiment, the method may target NAV1.7, NAV1.8 or NAV1.9 over at least NAV1.1, NAV1.2 and NAV1.5. In a further embodiment, the method may target NAV1.7, NAV1.8 or NAV1.9 over at least NAV1.1, NAV1.2 and NAV1.6. In a further embodiment, the method may target NAV1.7, NAV1.8 or NAV1.9 over at least NAV1.1, NAV1.3 and NAV1.4. In a further embodiment, the method may target NAV1.7, NAV1.8 or NAV1.9 over at least NAV1.1, NAV1.3 and NAV1.5. In a further embodiment, the method may target NAV1.7, NAV1.8 or NAV1.9 over at least NAV1.1, NAV1.3 and NAV1.6. In a further embodiment, the method may target NAV1.7, NAV1.8 or NAV1.9 over at least NAV1.1, NAV1.4 and NAV1.5. In a further embodiment, the method may target NAV1.7, NAV1.8 or NAV1.9 over at least NAV1.1, NAV1.4 and NAV1.6. In a further embodiment, the method may target NAV1.7, NAV1.8 or NAV1.9 over at least NAV1.2, NAV1.3 and NAV1.4. In a further embodiment, the method may target NAV1.7, NAV1.8 or NAV1.9 over at least NAV1.2, NAV1.3 and NAV1.5. In a further embodiment, the method may target NAV1.7, NAV1.8 or NAV1.9 over at least NAV1.2, NAV1.3 and NAV1.6. In a further embodiment, the method may target NAV1.7, NAV1.8 or NAV1.9 over at least NAV1.3, NAV1.4 and NAV1.5. In a further embodiment, the method may target NAV1.7, NAV1.8 or NAV1.9 over at least NAV1.3, NAV1.4 and NAV1.6.

The amount of a prophylactic or therapeutic agent (e.g., an anti-NAV protein antibody as described herein), or a composition as described herein that will be effective in the prevention, management, treatment and/or amelioration of a NAV protein-mediated disease can be determined by standard clinical techniques.

Accordingly, a dosage of an antibody or a composition that results in a serum titer of from about 0.1 μg/ml to about 450 μg/ml can be administered to a human for the prevention, management, treatment and/or amelioration of a NAV protein-mediated disease. In addition, in vitro assays may optionally be employed to help identify optimal dosage ranges. Effective doses may be extrapolated from dose-response curves derived from in vitro or animal model test systems.

The precise dose to be employed in the formulation will also depend on the route of administration, and the seriousness of a NAV protein-mediated disease, and should be decided according to the judgment of the practitioner and each patient's circumstances.

For the anti-NAV protein antibodies as disclosed herein, the dosage administered to a patient is typically 0.1 mg/kg to 100 mg/kg of the patient's body weight. In some embodiments, the dosage administered to the patient is about 1 mg/kg to about 75 mg/kg of the patient's body weight. Preferably, the dosage administered to a patient is between 1 mg/kg and 20 mg/kg of the patient's body weight, more preferably 1 mg/kg to 5 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the anti-NAV protein antibodies as disclosed herein may be reduced by enhancing uptake and tissue penetration of the antibodies by modifications such as, for example, lipidation.

In certain embodiments, anti-NAV protein antibodies as disclosed herein are administered prophylactically or therapeutically to a subject. Anti-NAV protein antibodies as disclosed herein can be prophylactically or therapeutically administered to a subject so as to prevent, lessen or ameliorate a NAV protein-mediated disease or symptom thereof.

### Gene Therapy

In a specific embodiment, nucleic acids or nucleotide sequences as disclosed herein are administered to prevent, manage, treat and/or ameliorate a NAV protein-mediated disease by way of gene therapy. Gene therapy refers to therapy performed by the administration to a subject of an expressed or expressible nucleic acid. In an embodiment, the nucleic acids produce their encoded antibody, and the antibody mediates a prophylactic or therapeutic effect.

Any of the methods for gene therapy available in the art can be used according to the methods disclosed herein.

### Diagnostic Use of Antibodies

Although antibodies (as described herein, in any embodiment or combination of embodiments) are mentioned in respect of diagnostic uses, this disclosure is to be read as also applying mutatis mutandis to the fragments of the invention.

Labelled antibodies as disclosed herein, which bind to a NAV protein antigen of interest can be used for diagnostic purposes to detect, diagnose, or monitor a NAV protein-mediated disease. There are provided methods for the detection of a NAV protein-mediated disease comprising: (a) assaying the expression of a NAV protein antigen in cells or a tissue sample of a subject using one or more anti-NAV protein antibodies as disclosed herein that bind to the NAV protein antigen; and (b) comparing the level of the NAV protein antigen of interest with a control level, e.g., levels in normal tissue samples (e.g., from a patient not having a NAV protein-mediated disease, or from the same patient before disease onset), whereby an increase in the assayed level of NAV protein antigen compared to the control level of the NAV protein antigen is indicative of a NAV protein-mediated disease.

There is provided a diagnostic assay for diagnosing a NAV protein-mediated disease comprising: (a) assaying for the level of a NAV protein antigen in cells or a tissue sample of an individual using one or more anti-NAV protein antibodies as disclosed herein that bind to a NAV protein antigen; and (b) comparing the level of the NAV protein antigen with a control level, e.g., levels in normal tissue samples, whereby an increase in the assayed NAV protein antigen level compared to the control level of the NAV protein antigen is indicative of a NAV protein-mediated disease. A more definitive diagnosis of a NAV protein-mediated disease may allow health professionals to employ preventative measures or aggressive treatment earlier thereby preventing the development or further progression of the NAV protein-mediated disease.

Anti-NAV antibodies as disclosed herein can be used to assay NAV protein antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Various conjugates as described hereinabove which may be of use in these assays.

In one aspect, there is provided the detection and diagnosis of a NAV protein-mediated disease in a human. In one embodiment, diagnosis comprises: a) administering (for example, parenterally, subcutaneously, or intraperitoneally) to a subject an effective amount of a labelled antibody that binds to a NAV protein antigen of interest; b) waiting for a time interval following the administering for permitting the labelled antibody to preferentially concentrate at sites in the subject where the NAV protein antigen is expressed (and for unbound labelled molecule to be cleared to background level); c) determining background level; and d) detecting the labelled antibody in the subject, such that detection of labelled antibody above the background level indicates that the subject has a NAV protein-mediated disease. Background level can be determined by various methods including, comparing the amount of labelled molecule detected to a standard value previously determined for a particular system.

In one embodiment, monitoring of a NAV protein-mediated disease is carried out by repeating the method for diagnosing the a NAV protein-mediated disease, for example, one month after initial diagnosis, six months after initial diagnosis, one year after initial diagnosis, etc.

Presence of the labelled molecule can be detected in the subject using methods known in the art for in vivo scanning. These methods depend upon the type of label used. Skilled artisans will be able to determine the appropriate method for detecting a particular label. Methods and devices that may be used in the diagnostic methods include, but are not limited to, computed tomography (CT), whole body scan such as position emission tomography (PET), magnetic resonance imaging (MRI), and sonography.

In a specific embodiment, the molecule is labelled with a radioisotope and is detected in the patient using a radiation responsive surgical instrument (Thurston et al., U.S. Pat. No. 5,441,050). In another embodiment, the molecule is labelled with a fluorescent compound and is detected in the patient using a fluorescence responsive scanning instrument. In another embodiment, the molecule is labelled with a positron emitting metal and is detected in the patient using positron emission-tomography. In yet another embodiment, the molecule is labelled with a paramagnetic label and is detected in a patient using magnetic resonance imaging (MRI).

Anti-NAV antibodies as disclosed herein may be used, for example, to purify, detect, and target NAV protein antigens, in both in vitro and in vivo diagnostic and therapeutic methods. For example, certain antibodies have use in immunoassays for qualitatively and quantitatively measuring levels of NAV protein in biological samples. See, e.g., Harlow et al., Antibodies: A Laboratory Manual, (Cold Spring Harbor Laboratory Press, 2nd ed. 1988) (incorporated by reference herein in its entirety)

### Kits

There is also provided a pharmaceutical or diagnostic pack or kit comprising one or more containers filled with one or more of the ingredients of the pharmaceutical compositions as disclosed herein, such as one or more anti-NAV antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration, e.g., an authorisation number.

There are provided kits that can be used in the above methods. In one embodiment, a kit comprises an anti-NAV antibody as disclosed herein, preferably a purified antibody, in one or more containers. In a specific embodiment, the kits contain a substantially isolated NAV protein antigen as a control. Preferably, the kits further comprise a control antibody which does not react with the NAV protein antigen of interest. In another specific embodiment, the kits contain a means for detecting the binding of an antibody to a NAV protein antigen (e.g., the antibody may be conjugated to a detectable substrate as described hereinabove, or a second antibody which recognizes the first antibody may be conjugated to a detectable substrate). In specific embodiments, the kit may include a recombinantly produced or chemically synthesized NAV protein antigen. The NAV protein antigen provided in the kit may also be attached to a solid support. In a more specific embodiment, the detecting means of the above described kit includes a solid support to which NAV protein antigen is attached. Such a kit may also include a non-attached reporter-labelled anti-human antibody. In this embodiment, binding of the antibody to the NAV protein antigen can be detected by binding of the said reporter-labelled antibody.

In one embodiment, there is provided a kit for treating and/or preventing a NAV-mediated condition or disease, the kit comprising an antibody or fragment as disclosed herein in any embodiment or combination of embodiments (and optionally an anti-nociceptive drug as described hereinabove) optionally in combination with a label or instructions for use to treat and/or prevent said disease or condition in a human; optionally wherein the label or instructions comprise a marketing authorisation number (e.g., an FDA or EMA authorisation number); optionally wherein the kit comprises an IV or injection device that comprises the antibody or fragment.

It will be understood that particular configurations, aspects, examples, clauses and embodiments described herein are shown by way of illustration and not as limitations of the invention. Any part of this disclosure may be read in combination with any other part of the disclosure, unless otherwise apparent from the context.

While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended sentences.

### EXAMPLES

### Sequence Alignment of Human NAV Proteins—NAV1.1 to NAV1.9

Multiple protein sequences of Nav family members were aligned using the algorithm, Clustal W method (Thompson, J. D. et al. Nucleic Acids Research, 1994, 22: 4673-4680). The new loop was based on the regions between S1-52, S3-S4 and S5-S6 regions of annotated Nav1.7 sequence shown in the Supplementary Table S3 (Yang, Y et al. Nat Commun. 2012. 3: 1186).

### Example 1—Antigen Preparation, Immunization Procedures, and Hybridoma Generation

The following example provides a detailed description of the generation and identification of a panel of anti-human NAV1.7 monoclonal antibodies using the Kymouse™ system (see, eg, WO2011/004192). To this end, genetically engineered mice containing a large number of human immunoglobulin genes were immunized with human NAV1.7 E2 loop polypeptides and surface expressed human NAV1.7 displayed on Human Embryonic Kidney (HEK) cells. Various immunization regimes, involving a mixture of peptide and HEK boosters were set up, boosting animals over several weeks (see detailed methods below). At the end of each regime, secondary lymphoid tissue such as the spleen, and in some cases, the lymph nodes are removed. Tissues are prepared into a single cell suspension and fused with SP2/0 cells to generate a stable hybridoma cell line.

### Materials and Methods

### A: Generation of Stably Transfected HEK Cells Expressing Human NAV1.7:

The full length human NAV1.7 sequences were codon optimized for mammalian expression and cloned into an expression vector under the CMV promoter flanked by 3′ and 5′ piggyBac specific terminal repeat sequences facilitating stable integration into the cell genome (see: “A hyperactive piggyBac transposase for mammalian applications”; Yusa K, Zhou L, Li M A, Bradley A, Craig N L. Proc Natl Acad Sci USA. 2011 Jan. 25). Furthermore, the expression vector contained either a puromycin or neomycin selection cassette to facilitate stable cell line generation. The NAV protein expression plasmid was co-transfected with a plasmid encoding piggyBac transposase into a HEK293 cell line (available from Invitrogen) using the FreeStyle Max transfection reagent (Invitrogen) according to manufacturer instructions. 24 hours after transfection, the media was supplemented with puromycin or G418 and grown for at least 1 week to select a stable cell line with media being exchanged every 3-4 days. The expression of NAV protein was assessed by RNA expression levels, and functional assays conducted according to Example 2G below. Cells were stored in HEK media until use.

### B: Preparation of HEK Cells for Mouse Immunizations:

Cell culture medium was removed and cells washed once with 1×PBS. Cells were treated for 5 minutes with trypsin to loosen cells from tissue culture surface. Cells were collected and trypsin neutralized by the addition of complete media containing 10% v/v fetal bovine serum (FCS). Cells were then centrifuged at 300×g for 10 minutes and washed with 25 ml of 1×PBS. Cells were counted and resuspended at the appropriate concentration in 1×PBS.

### C: Immunization Procedure and Schedules:

Transgenic Kymice™ were immunized with HEK cells expressing NAV protein (as in Example 1A above). For each peptide, the KLH carrier is conjugated through the cysteine. For cyclic peptides (as in SEQ ID No: 14, 28, 42 and 54), the cyclisation occurs at the cysteine, which is also conjugated to the KLH. All mice were bled before being primed and then boosted according to the schedules below. In each group, there were 12 to 37 days between each injection. Mice were bled 7 days after certain booster injections (generally after the second and third boosts) and analysed for NAV protein specific IgG titre using an ELISA (based on the peptide-conjugate sequences), flow cytometry based assay (based on the HEK cells expressing the NAV protein of interest) and functional assays conducted according to Example 2G below. Final boosts were administered 3-5 days prior to tissue collection. 50 μl, 100 μl or 200 μl was administered on each dosing.

Titre data is used to select mice for fusion and hybridoma generation. Spleen tissues or lymph nodes are taken and subjected to hybridoma fusion or B-cell cloning. Briefly, B cell cloning is carried out as follows. B cells are probed with biotin-labelled peptide antigen and single-cell sorted. Subsequently, RT-PCR is carried out on each cell, followed by bridge PCR to generate expression cassettes. The expression cassettes are expressed in HEK cells and screened for the desired properties.

Mice were divided into seven groups according to immunization procedure and immunised as follows:

Group one as in Table 2.

Group two as in Table 3.

Group three as in Table 4.

Group four as in Table 5.

Group 5 as in Table 6.

Further immunisations may be carried out using other antigens as appropriate. Intramuscular DNA immunisations may be carried out by co-injection with the NAV protein expression plasmid (as described in Example 1A) and a plasmid encoding piggyBac transposase. Mice may be injected with a priming dose, followed by two boosters.

Alternatively, mice may be immunised with the carrier-conjugated peptide antigens alone (i.e. without any immunisation with HEK cells expressing the NAV protein), such as those peptide antigens in SEQ ID No: 6, 10, 14, 18, 24, 28, 32, 36, 42, 46, 50 and 54, or any other peptide derived from an external loop of the NAV protein of interest). The mice are primed intraperitoneally with an adjuvanted dose (either containing one, two or three [such as KLH-conjugated peptide at the C-terminus, at the N-terminus and cyclic peptide] conjugated peptide antigens), and then boosted intraperitoneally at least 3 times (or 4 times or 5 times). The final booster dose is administered intravenously without any adjuvant. The adjuvant used may be CFA/Sigma with Alum and CpG in the priming dose, and may be either IFA/Sigma with Alum and CpG for the booster doses.

Alternatively, mice may be immunised with HEK cells expressing the NAV protein of interest alone (i.e. without any immunisation with carrier-conjugated peptide antigens). The mice are primed intraperitoneally with or without Sigma adjuvant, followed by at least 3 (or 4 or 5) intraperitoneal booster doses, also with or without Sigma adjuvant. The final booster dose is administered intraperitoneally without any adjuvant.

Alternatively, mice may be immunised using the Rapid Immunisation at Multiple Sites (RIMS) procedure as described in “Rapid Development of Affinity Matured Monoclonal Antibodies Using RIMMS”; Kilpatrick et al. Hybridoma, 1997.

TABLE 2

| | Prime, IP¹ | | Boost 1, IP | | Boost 2, IP | | Boost 3, IP | | Boost 4, IP | | Boost 5, IV² | |
|---|---|---|---|---|---|---|---|---|---|---|---|---|
| | | Adju- | | Adju- | | Adju- | | Adju- | | Adju- | | Adju- |
| Mouse | Antigen | vant | Antigen | vant | Antigen | vant | Antigen | vant | Antigen | vant | Antigen | vant |
| 1 | 10⁷ | Sigma³ | 10 μg KP1.1⁴ | Sigma⁶ | 2 × 10⁶ | no | 3 μg KP1.1 | Sigma | 2 × 10⁶ | no | 0.5 μg KP1.1 | no |
| | hNav1.7 | | peptide-KLH | CpG⁷ | hNav1.7 | | peptide-KLH | CpG | hNav1.7 | | peptide-KLH | |
| | HEKs | | 10 μg KP1.2⁵ | Alum⁸ | HEKs | | 3 μg KP1.2 | Alum | HEKs | | 0.5 μg KP1.2 | |
| | | | peptide-KLH | | | | peptide-KLH | | | | peptide-KLH | |
| 2 | 10⁷ | Sigma | 10 μg KP1.1 | Sigma | 2 × 10⁶ | no | 3 μg KP1.1 | Sigma | 2 × 10⁶ | no | 0.5 μg KP1.1 | no |
| | hNav1.7 | | peptide-KLH | CpG | hNav1.7 | | peptide-KLH; | CpG | hNav1.7 | | peptide-KLH | |
| | HEKs | | 10 μg KP1.2 | Alum | HEKs | | 3 μg KP1.2 | Alum | HEKs | | 0.5 μg KP1.2 | |
| | | | peptide-KLH | | | | peptide-KLH | | | | peptide-KLH | |
| 3 | 10⁷ | Sigma | 10 μg KP1.1 | Sigma | 2 × 10⁶ | no | 3 μg KP1.1 | Sigma | 2 × 10⁶ | no | 0.5 μg KP1.1 | no |
| | hNav1.7 | | peptide-KLH | CpG | hNav1.7 | | peptide-KLH; | CpG | hNav1.7 | | peptide-KLH | |
| | HEKs | | 10 μg KP1.2 | Alum | HEKs | | 3 μg KP1.2 | Alum | HEKs | | 0.5 μg KP1.2 | |
| | | | peptide-KLH | | | | peptide-KLH | | | | peptide-KLH | |
| 4 | 10⁷ | Sigma | 10 μg KP1.1 | Sigma | 2 × 10⁶ | no | 3 μg KP1.1 | Sigma | 2 × 10⁶ | no | 0.5 μg KP1.1 | no |
| | hNav1.7 | | peptide-KLH | CpG | hNav1.7 | | peptide-KLH; | CpG | hNav1.7 | | peptide-KLH | |
| | HEKs | | 10 μg KP1.2 | Alum | HEKs | | 3 μg KP1.2 | Alum | HEKs | | 0.5 μg KP1.2 | |
| | | | peptide-KLH | | | | peptide-KLH | | | | peptide-KLH | |
| 5 | 10⁷ | Sigma | 10 μg KP1.1 | Sigma | 2 × 10⁶ | no | 3 μg KP1.1 | Sigma | 2 × 10⁶ | no | 0.5 μg KP1.1 | no |
| | hNav1.7 | | peptide-KLH | CpG | hNav1.7 | | peptide-KLH; | CpG | hNav1.7 | | peptide-KLH | |
| | HEKs | | 10 μg KP1.2 | Alum | HEKs | | 3 μg KP1.2 | Alum | HEKs | | 0.5 μg KP1.2 | |
| | | | peptide-KLH | | | | peptide-KLH | | | | peptide-KLH | |
| 6 | 10⁷ | Sigma | 10 μg KP1.1 | Sigma | 2 × 10⁶ | no | 3 μg KP1.1 | Sigma | 2 × 10⁶ | no | 0.5 μg KP1.1 | no |
| | hNav1.7 | | peptide-KLH | CpG | hNav1.7 | | peptide-KLH | CpG | hNav1.7 | | peptide-KLH | |
| | HEKs | | 10 μg KP1.2 | Alum | HEKs | | 3 μg KP1.2 | Alum | HEKs | | 0.5 μg KP1.2 | |
| | | | peptide-KLH | | | | peptide-KLH | | | | peptide-KLH | |

¹IP = intraperitoneally
²IV = intravenously
³Sigma (S6322) concentration of 10-50% (v/v)
⁴KP1.1 = SEQ ID No: 6
⁵KP1.2 = SEQ ID No: 10
⁶Sigma (S6322) concentration of 2% (v/v)
⁷CpG concentration of 0.1 mg/ml of oligodeoxynucleotide ODN 1826
⁸Alum concentration of 25% Alhydrogel 2% (v/v)

TABLE 3

| | Prime, IP | | Boost 1, IP | | Boost 2, IP | | Boost 3, IP | | Boost 4, IP | | Boost 5, IV | |
|---|---|---|---|---|---|---|---|---|---|---|---|---|
| | | Adju- | | Adju- | | Adju- | | Adju- | | Adju- | | Adju- |
| Mouse | Antigen | vant | Antigen | vant | Antigen | vant | Antigen | vant | Antigen | vant | Antigen | vant |
| 1 | 10⁷ | Sigma¹ | 20 μg KP2.3² | Sigma³ | 2 × 10⁶ | no | 5 μg KP2.3 | Sigma | 2 × 10⁶ | no | 1 μg KP2.3 | no |
| | hNav1.7 | | peptide-KLH | CpG⁴ | hNav1.7 | | peptide-KLH | CpG | hNav1.7 | | peptide-KLH | |
| | HEKs | | | Alum⁵ | HEKs | | | Alum | HEKs | | | |
| 2 | 10⁷ | Sigma | 20 μg KP2.3 | Sigma | 2 × 10⁶ | no | 5 μg KP2.3 | Sigma | 2 × 10⁶ | no | 1 μg KP2.3 | no |
| | hNav1.7 | | peptide-KLH | CpG | hNav1.7 | | peptide-KLH | CpG | hNav1.7 | | peptide-KLH | |
| | HEKs | | | Alum | HEKs | | | Alum | HEKs | | | |
| 3 | 10⁷ | Sigma | 20 μg KP2.3 | Sigma | 2 × 10⁶ | no | 5 μg KP2.3 | Sigma | 2 × 10⁶ | no | 1 μg KP2.3 | no |
| | hNav1.7 | | peptide-KLH | CpG | hNav1.7 | | peptide-KLH | CpG | hNav1.7 | | peptide-KLH | |
| | HEKs | | | Alum | HEKs | | | Alum | HEKs | | | |
| 4 | 10⁷ | Sigma | 20 μg KP2.3 | Sigma | 2 × 10⁶ | no | 5 μg KP2.3 | Sigma | 2 × 10⁶ | no | 1 μg KP2.3 | no |
| | hNav1.7 | | peptide-KLH | CpG | hNav1.7 | | peptide-KLH | CpG | hNav1.7 | | peptide-KLH | |
| | HEKs | | | Alum | HEKs | | | Alum | HEKs | | | |
| 5 | 10⁷ | Sigma | 20 μg KP2.3 | Sigma | 2 × 10⁶ | no | 5 μg KP2.3 | Sigma | 2 × 10⁶ | no | 1 μg KP2.3 | no |
| | hNav1.7 | | peptide-KLH | CpG | hNav1.7 | | peptide-KLH | CpG | hNav1.7 | | peptide-KLH | |
| | HEKs | | | Alum | HEKs | | | Alum | HEKs | | | |
| 6 | 10⁷ | Sigma | 20 μg KP2.3 | Sigma | 2 × 10⁶ | no | 5 μg KP2.3 | Sigma | 2 × 10⁶ | no | 1 μg KP2.3 | no |
| | hNav1.7 | | peptide-KLH | CpG | hNav1.7 | | peptide-KLH | CpG | hNav1.7 | | peptide-KLH | |
| | HEKs | | | Alum | HEKs | | | Alum | HEKs | | | |

¹Sigma (S6322) concentration of 10-50% (v/v)
²KP2.3 = SEQ ID No: 28
³Sigma (S6322) concentration of 2% (v/v)
⁴CpG concentration of 0.1 mg/ml of oligodeoxynucleotide ODN 1826
⁵Alum concentration of 25% Alhydrogel 2% (v/v)

TABLE 4

| | Prime, IP | | | | | | |
|---|---|---|---|---|---|---|---|
| | | Adju- | Boost 1, IP | | Boost 2, IP | | Boost 3, IP |
| Mouse | Antigen | vant | Antigen | Adj | Antigen | Adj | Antigen |
| 1 | 10⁷ | Sigma¹ | 10 μg KP5.1² | Sigma⁴ | 2 × 10⁶ | no | 3 μg KP5.1 |
| | hNav1.7 | | peptide-KLH | CpG⁵ | hNav1.7 | | peptide-KLH |
| | HEKs | | 10 μg KP5.3³ | Alum⁶ | HEKs | | 3 μg KP5.3 |
| | | | peptide-KLH | | | | peptide-KLH |
| 2 | 10⁷ | Sigma | 10 μg KP5.1 | Sigma | 2 × 10⁶ | no | 3 μg KP5.1 |
| | hNav1.7 | | peptide-KLH | CpG | hNav1.7 | | peptide-KLH |
| | HEKs | | 10 μg KP5.3 | Alum | HEKs | | 3 μg KP5.3 |
| | | | peptide-KLH | | | | peptide-KLH |
| 3 | 10⁷ | Sigma | 10 μg KP5.1 | Sigma | 2 × 10⁶ | no | 3 μg KP5.1 |
| | hNav1.7 | | peptide-KLH | CpG | hNav1.7 | | peptide-KLH |
| | HEKs | | 10 μg KP5.3 | Alum | HEKs | | 3 μg KP5.3 |
| | | | peptide-KLH | | | | peptide-KLH |
| 4 | 10⁷ | Sigma | 10 μg KP5.1 | Sigma | 2 × 10⁶ | no | 3 μg KP5.1 |
| | hNav1.7 | | peptide-KLH | CpG | hNav1.7 | | peptide-KLH |
| | HEKs | | 10 μg KP5.3 | Alum | HEKs | | 3 μg KP5.3 |
| | | | peptide-KLH | | | | peptide-KLH |
| 5 | 10⁷ | Sigma | 10 μg KP5.1 | Sigma | 2 × 10⁶ | no | 3 μg KP5.1 |
| | hNav1.7 | | peptide-KLH | CpG | hNav1.7 | | peptide-KLH |
| | HEKs | | 10 μg KP5.3 | Alum | HEKs | | 3 μg KP5.3 |
| | | | peptide-KLH | | | | peptide-KLH |

| | Boost 3, IP | Boost 4, IP | | Boost 5, IP | | Boost 6, IV | |
|---|---|---|---|---|---|---|---|
| Mouse | Adj | Antigen | Adj | Antigen | Adj | Antigen | Adj |
| 1 | Sigma | 2 × 10⁶ | no | 3 μg KP5.1 | Sigma | 0.5 μg KP5.1 | no |
| | CpG | hNav1.7 | | peptide-KLH | CpG | peptide-KLH | |
| | Alum | HEKs | | 3 μg KP5.3 | Alum | 0.5 μg KP5.3 | |
| | | | | peptide-KLH | | peptide-KLH | |
| 2 | Sigma | 2 × 10⁶ | no | 3 μg KP5.1 | Sigma | 0.5 μg KP5.1 | no |
| | CpG | hNav1.7 | | peptide-KLH | CpG | peptide-KLH | |
| | Alum | HEKs | | 3 μg KP5.3 | Alum | 0.5 μg KP5.3 | |
| | | | | peptide-KLH | | peptide-KLH | |
| 3 | Sigma | 2 × 10⁶ | no | 3 μg KP5.1 | Sigma | 0.5 μg KP5.1 | no |
| | CpG | hNav1.7 | | peptide-KLH | CpG | peptide-KLH | |
| | Alum | HEKs | | 3 μg KP5.3 | Alum | 0.5 μg KP5.3 | |
| | | | | peptide-KLH | | peptide-KLH | |
| 4 | Sigma | 2 × 10⁶ | no | 3 μg KP5.1 | Sigma | 0.5 μg KP5.1 | no |
| | CpG | hNav1.7 | | peptide-KLH | CpG | peptide-KLH | |
| | Alum | HEKs | | 3 μg KP5.3 | Alum | 0.5 μg KP5.3 | |
| | | | | peptide-KLH | | peptide-KLH | |
| 5 | Sigma | 2 × 10⁶ | no | 3 μg KP5.1 | Sigma | 0.5 μg KP5.1 | no |
| | CpG | hNav1.7 | | peptide-KLH | CpG | peptide-KLH | |
| | Alum | HEKs | | 3 μg KP5.3 | Alum | 0.5 μg KP5.3 | |
| | | | | peptide-KLH | | peptide-KLH | |

¹Sigma (S6322) concentration of 10-50% (v/v)
²KP5.1 = SEQ ID No: 32
³KP5.2 = SEQ ID No: 42
⁴Sigma (S6322) concentration of 2% (v/v)
⁵CpG concentration of 0.1 mg/ml of oligodeoxynucleotide ODN 1826
⁶Alum concentration of 25% Alhydrogel 2% (v/v)

TABLE 5

| | Prime, IP | | | | | | |
|---|---|---|---|---|---|---|---|
| | | Adju- | Boost 1, IP | | Boost 2, IP | | Boost 3, IP |
| Mouse | Antigen | vant | Antigen | Adj | Antigen | Adj | Antigen |
| 1 | 10⁷ | Sigma¹ | 10 μg KP6.1² | Sigma⁴ | 5 × 10⁶ | no | 3 μg KP6.1 |
| | hNav1.7 | | peptide-KLH | CpG⁵ | hNav1.7 | | peptide-KLH |
| | HEKs | | 10 μg KP6.3³ | Alum⁶ | HEKs | | 3 μg KP6.3 |
| | | | peptide-KLH | | | | peptide-KLH |
| 2 | 10⁷ | Sigma | 10 μg KP6.1 | Sigma | 5 × 10⁶ | no | 3 μg KP6.1 |
| | hNav1.7 | | peptide-KLH | CpG | hNav1.7 | | peptide-KLH |
| | HEKs | | 10 μg KP6.3 | Alum | HEKs | | 3 μg KP6.3 |
| | | | peptide-KLH | | | | peptide-KLH |
| 3 | 10⁷ | Sigma | 10 μg KP6.1 | Sigma | 5 × 10⁶ | no | 3 μg KP6.1 |
| | hNav1.7 | | peptide-KLH | CpG | hNav1.7 | | peptide-KLH |
| | HEKs | | 10 μg KP6.3 | Alum | HEKs | | 3 μg KP6.3 |
| | | | peptide-KLH | | | | peptide-KLH |
| 4 | 10⁷ | Sigma | 10 μg KP6.1 | Sigma | 5 × 10⁶ | no | 3 μg KP6.1 |
| | hNav1.7 | | peptide-KLH | CpG | hNav1.7 | | peptide-KLH |
| | HEKs | | 10 μg KP6.3 | Alum | HEKs | | 3 μg KP6.3 |
| | | | peptide-KLH | | | | peptide-KLH |
| 5 | 10⁷ | Sigma | 10 μg KP6.1 | Sigma | 5 × 10⁶ | no | 3 μg KP6.1 |
| | hNav1.7 | | peptide-KLH | CpG | hNav1.7 | | peptide-KLH |
| | HEKs | | 10 μg KP6.3 | Alum | HEKs | | 3 μg KP6.3 |
| | | | peptide-KLH | | | | peptide-KLH |

| | Boost 3, IP | Boost 4, IP | | Boost 5, IP | | Boost 6, IV | |
|---|---|---|---|---|---|---|---|
| Mouse | Adj | Antigen | Adj | Antigen | Adj | Antigen | Adj |
| 1 | Sigma | 5 × 10⁶ | no | 3 μg KP6.1 | Sigma | 0.5 μg KP6.1 | no |
| | CpG | hNav1.7 | | peptide-KLH | CpG | peptide-KLH | |
| | Alum | HEKs | | 3 μg KP6.3 | Alum | 0.5 μg KP6.3 | |
| | | | | peptide-KLH | | peptide-KLH | |
| 2 | Sigma | 5 × 10⁶ | no | 3 μg KP6.1 | Sigma | 0.5 μg KP6.1 | no |
| | CpG | hNav1.7 | | peptide-KLH | CpG | peptide-KLH | |
| | Alum | HEKs | | 3 μg KP6.3 | Alum | 0.5 μg KP6.3 | |
| | | | | peptide-KLH | | peptide-KLH | |
| 3 | Sigma | 5 × 10⁶ | no | 3 μg KP6.1 | Sigma | 0.5 μg KP6.1 | no |
| | CpG | hNav1.7 | | peptide-KLH | CpG | peptide-KLH | |
| | Alum | HEKs | | 3 μg KP6.3 | Alum | 0.5 μg KP6.3 | |
| | | | | peptide-KLH | | peptide-KLH | |
| 4 | Sigma | 5 × 10⁶ | no | 3 μg KP6.1 | Sigma | 0.5 μg KP6.1 | no |
| | CpG | hNav1.7 | | peptide-KLH | CpG | peptide-KLH | |
| | Alum | HEKs | | 3 μg KP6.3 | Alum | 0.5 μg KP6.3 | |
| | | | | peptide-KLH | | peptide-KLH | |
| 5 | Sigma | 5 × 10⁶ | no | 3 μg KP6.1 | Sigma | 0.5 μg KP6.1 | no |
| | CpG | hNav1.7 | | peptide-KLH | CpG | peptide-KLH | |
| | Alum | HEKs | | 3 μg KP6.3 | Alum | 0.5 μg KP6.3 | |
| | | | | peptide-KLH | | peptide-KLH | |

¹Sigma (S6322) concentration of 10-50% (v/v)
²KP6.1 = SEQ ID No: 46
³KP6.3 = SEQ ID No: 54
⁴Sigma (S6322) concentration of 2% (v/v)
⁵CpG concentration of 0.1 mg/ml of oligodeoxynucleotide ODN 1826
⁶Alum concentration of 25% Alhydrogel 2% (v/v)

TABLE 6

| | Prime, IP | | Boost 1, IP | | Boost 2, IP | | Boost 3, IP |
|---|---|---|---|---|---|---|---|
| Mouse | Antigen | Adj | Antigen | Adj | Antigen | Adj | Antigen |
| 1 | 20 μg KP1.2 | CFA¹ | 5 μg KP1.2⁴ | IFA⁶ | 5 × 10⁶ | Sigma ⁷ | 3 μg KP1.2 |
| | peptide-KLH | CpG² | peptide-KLH | CpG² | hNav1.7 | | peptide-KLH |
| | 20 μg KP5.1 | Alum³ | 5 μg KP5.1⁵ | Alum³ | HEKs | | 3 μg KP5.1 |
| | peptide-KLH | | peptide-KLH | | | | peptide-KLH |
| 2 | 20 μg KP1.2 | CFA | 5 μg KP1.2 | IFA | 5 × 10⁶ | Sigma | 3 μg KP1.2 |
| | peptide-KLH | CpG | peptide-KLH | CpG | hNav1.7 | | peptide-KLH |
| | 20 μg KP5.1 | Alum | 5 μg KP5.1 | Alum | HEKs | | 3 μg KP5.1 |
| | peptide-KLH | | peptide-KLH | | | | peptide-KLH |
| 3 | 20 μg KP1.2 | CFA | 5 μg KP1.2 | IFA | 5 × 10⁶ | Sigma | 3 μg KP1.2 |
| | peptide-KLH | CpG | peptide-KLH | CpG | hNav1.7 | | peptide-KLH |
| | 20 μg KP5.1 | Alum | 5 μg KP5.1 | Alum | HEKs | | 3 μg KP5.1 |
| | peptide-KLH | | peptide-KLH | | | | peptide-KLH |
| 4 | 20 μg KP1.2 | CFA | 5 μg KP1.2 | IFA | 5 × 10⁶ | Sigma | 3 μg KP1.2 |
| | peptide-KLH | CpG | peptide-KLH | CpG | hNav1.7 | | peptide-KLH |
| | 20 μg KP5.1 | Alum | 5 μg KP5.1 | Alum | HEKs | | 3 μg KP5.1 |
| | peptide-KLH | | peptide-KLH | | | | peptide-KLH |

| | Boost 3, IP | Boost 4, IP | | Boost 5, IP | | Boost 6, IV | |
|---|---|---|---|---|---|---|---|
| Mouse | Adj | Antigen | Adj | Antigen | Adj | Antigen | Adj |
| 1 | IFA | 2 × 10⁶ | Sigma | 3 μg KP1.2 | IFA | 1 μg KP1.2 | no |
| | CpG | hNav1.7 | | peptide-KLH | CpG | peptide-KLH | |
| | Alum | HEKs | | 3 μg KP5.1 | Alum | 1 μg KP5.1 | |
| | | | | peptide-KLH | | peptide-KLH | |
| 2 | IFA | 2 × 10⁶ | Sigma | 3 μg KP1.2 | IFA | 1 μg KP1.2 | no |
| | CpG | hNav1.7 | | peptide-KLH | CpG | peptide-KLH | |
| | Alum | HEKs | | 3 μg KP5.1 | Alum | 1 μg KP5.1 | |
| | | | | peptide-KLH | | peptide-KLH | |
| 3 | IFA | 2 × 10⁶ | Sigma | 3 μg KP1.2 | IFA | 1 μg KP1.2 | no |
| | CpG | hNav1.7 | | peptide-KLH | CpG | peptide-KLH | |
| | Alum | HEKs | | 3 μg KP5.1 | Alum | 1 μg KP5.1 | |
| | | | | peptide-KLH | | peptide-KLH | |
| 4 | IFA | 2 × 10⁶ | Sigma | 3 μg KP1.2 | IFA | 1 μg KP1.2 | no |
| | CpG | hNav1.7 | | peptide-KLH | CpG | peptide-KLH | |
| | Alum | HEKs | | 3 μg KP5.1 | Alum | 1 μg KP5.1 | |
| | | | | peptide-KLH | | peptide-KLH | |

¹Complete Freud's Adjuvant at a concentration of 50% (v/v)
²CpG concentration of 0.1 mg/ml of oligodeoxynucleotide ODN 1826
³Alum concentration of 25% Alhydrogel 2% (v/v)
⁴KP1.2 = SEQ ID No: 10
⁵KP5.1 = SEQ ID No: 32
⁶Incomplete Freud's Adjuvant at a concentration of 50% (v/v)
⁷ Sigma (S6322) concentration of 2% (v/v)

### D: Murine Tissue Isolation and Preparation:

Spleens are excised from immunised mice and washed in 1×PBS and kept on ice until further processing. Where used, axillary, inguinal as well as mesenteric lymph nodes are removed and placed in sterile 1×PBS on ice until further processing. Tissues are prepared in buffer containing 1×PBS (Invitrogen) and 3% heat-inactivated FBS (Invitrogen). Splenocytes are dispersed by mashing the tissue through a 45 μm strainer (BD Falcon) and rinsing with ˜30 ml 3% FBS/PBS buffer before centrifugation at ˜700 g for ˜10 minutes at 4° C. To remove red blood cells, the pelleted splenocytes are resuspended in ˜4 ml of Red Blood Cell Lysis Buffer (Sigma). After ˜4 minutes of incubation, the lysis reaction is stopped by addition of 3% FBS/1×PBS buffer. Cell clumps are filtered out with a 45 μm strainer. The remaining splenocytes and lymph node cells are pelleted for further procedures.

### E: Hybridoma Fusion:

Following final boost, spleens or lymph nodes are taken and B-cells subjected to a positive selection method using the MACS® Separation system. Briefly, where lymph nodes are used, those cells are pooled with the splenocytes from the corresponding mice after red blood cell lysis and total cell number determined. Cells are resuspended in 80 μl 3% FBS/PBS buffer per 1×10⁷ cells, before adding the anti-mouse IgG1 plus anti-mouse IgG2a+b MicroBeads (Miltenyi Biotec) and incubated for ˜15 minutes at ˜4° C. The cells/MicroBeads mixture are then applied to a pre-wetted LS column placed in a magnetic MACS Separator and washed with 3% FBS/PBS buffer. IgG positive cells are collected in the labelled, column-bound fraction in 3% FBS/PBS buffer.

Enriched B-cells are treated with CpG overnight (final concentration 25 pM) and the following day washed once in BSA fusion buffer (0.3M D-Sorbitol, 0.11 mM calcium acetate hydrate, 0.5 mM magnesium acetate tetrahydrate and 0.1% BSA (v/w), adjusted to pH7.2). Washed cells are resuspended in 200 μl of BSA fusion buffer and cell count determined. SP2/0 cells are treated in the same way, but washed twice instead of once with BSA fusion buffer. B-cells are fused at a ratio of 3:1 with SP2/0 myeloma cells by electrofusion using a BTX ECM 2001 Electro Cell Manipulator (Harvard Apparatus). Each fusion is left overnight in recovery medium (Dulbecco's Modified Eagle's Medium—high glucose (no phenol red, no L-G) containing OPI (Sigma), L-Glutamax (Gibco), 20% FBS (Gibco, batch-tested for hybridoma) and 2-mercaptoethanol, resuspended in 1 part recovery medium and 9 parts semi-solid medium (ClonaCell-HY Hybridoma Selection Medium D, Stemcell Technologies) and then seeded onto 10 cm petri dishes. Visible colonies are picked 12 days later into 96-well plates and cultured for another 2-3 days prior to screening.

### F: Monocloning of Hybridoma Wells:

Hybridomas found to be polyclonal are monocloned using the following procedure. Cells taken from an existing colony growing in one well of a 24 well plate are counted using Trypan Blue exclusion on the Cedex cell counter and seeded at a final concentration of 400 viable cells/mil in 1 part Hybridoma Maintenance Medium (Advanced DMEM, L-Glutamax, 20% FBS (Gibco, batch-tested for hybridoma), HT supplement, Penicillin-Streptomycin and 2-mercaptoethanol) to 9 parts semi-solid medium (ClonaCell-HY Hybridoma Selection Medium D) onto 10 cm petri dishes. Visible colonies are picked 12 days later into 96 well plates and cultured for another 2-3 days prior to screening. A maximum of 4×96 well plates are picked per parental colony and screened for NAV protein binding and functionality. The best monocloned hybridoma clone is taken forward.

### G: Hybridoma Supernatant Screening

After generation of hybridoma clones, the hybridoma supernatant is assessed in a sequential primary and secondary screen and appropriate hybridoma clones selected based on criteria of antibody binding to NAV protein and functional activity in a Patch Clamp assay.

Alternatively, peptide-specific B cells from immunized mice are single cell sorted, and the antibody sequence is rescued by RT-PCR, bridged to expression cassette, and expressed in HEK cells for the similar screening as listed above.

### Example 2: In Vitro Characterisation of Antibodies

### 2A: Binding Affinities by SPR

The binding affinities of anti-NAV antibodies to NAV loop peptides may be determined by Biacore.

For monovalent kinetic experiments, antibodies are captured through the Fc region via a goat anti-Fc-coupled biosensor surface, and the peptides (as analyte, e.g. SEQ ID No: 4, 6, 8, 10, 12, 14, 16, 18, 22, 24, 26, 28, 30, 32, 34, 36, 40, 42, 44, 46, 48, 50, 52, 54, 86, 90, 96, 100, 104, 108, 114, 158, 169, 171, 173, 176, 178, 180, 183, 185, 187, 191, 193, 196, 198, 200, 202-210, 212, 215, 218, 221, 223, 225, 227, 229, 231, 237, 243, 246, 249, 259-261, 264-266, 275, 279, 280, 282-284, 293-298, 300-302, 305, 307, 308, 310, 312, 318, 319, 321, 324, 325, 328 (in particular SEQ ID No: 6, 10, 14, 18, 24, 28, 32, 36, 42, 46, 50, 54) are injected over this captured antibody surface.

For bivalent (avidity-driven) kinetic experiments, biotinylated peptides corresponding to the NAV loop sequences (e.g. for NAV1.7, SEQ ID NOs: 4, 8, 12 and 16; for NAV1.8, SEQ ID No:22, 26, 30 and 34; and for NAV1.9, SEQ ID No: 40, 44, 48 and 52), or the biotinylated peptides corresponding to the loop motifs (e.g. for NAV 1.7, SEQ ID NOs: 6, 10, 14, 18, 24, 28, 32, 36, 42, 46, 50, 54, 100, 104, 108, 114, 158, 162, 169, 171, 173, 176, 178, 180, 183, 185, 187, 191, 193, 198, 200, 203, 205, 208, 210, 212, 215, 218, 221, 223, 225, 227, 229, 231, 237, 243, 259, 279, 283, 293, 300, 305, 307, 308, 318, 319 and 325 (in particular SEQ ID No: 6, 10, 14, 18, 24, 28, 32, 36, 42, 46, 50, 54); for NAV1.8, SEQ ID No: 90, 104, 108, 162, 196, 202, 204, 206, 234, 236, 241, 242, 244, 246, 249, 264, 265, 266, 280, 282, 284, 296, 310, 321, 324 and 328; and for NAV1.9, SEQ ID No: 86, 96, 207, 209, 260, 261, 275, 294, 295, 297, 298, 301, 302 and 312) are captured on a NeutrAvidin sensor surface, and anti-NAV antibodies (as analyte) are injected over this surface. Following the capture step, each analyte is individually injected at several concentrations over its respective capture surface, and changes in bound surface units (RU) are monitored. The dissociation rate is independent of the concentration of analyte used in the experiment, and the dissociation rate constant (kd) is determined from the change in antibody-bound analyte RU over time. The Biacore kinetic data is obtained using a double referencing procedure. The double referencing is conducted by first subtracting any interaction of the analyte over the reference surface (i.e., the anti-Fe coupled surface alone or the NeutrAvidin surface alone), thereby correcting for nonspecific binding to capture surface and for refractive index changes. Control buffer injections (no analyte) over the antibody- or peptide-captured surfaces are also performed to allow subtraction of RU signal changes resulting from the natural dissociation of captured binding partner from the sensor surface. The kinetic parameters are obtained by globally fitting the data for all concentrations tested for a given peptide or antibody to a 1:1 binding model using Biacore T100 Evaluation Software version 2.0.2. The K_{D} was calculated as the dissociation rate constant divided by the association rate constant (K_{D}=K_{D}/K_{A}). The dissociative half-life (t_{1/2}) was calculated from the dissociation rate constant (t_{1/2}=ln 2/k_{D}).

Synthetic peptides may be purchased for any appropriate supplier. For biotinylated forms, biotin moieties may be covalently attached to the peptide at either the C-terminus or the N-terminus via a G₄S linker.

Peptides from other species (e.g. mouse, rat, cyno) may be used to determine binding specificity.

### 2Aa: Antibody Binding by DELFIA Assay

Four mice were immunised substantially as detailed in Table 6. The immunisations of Boost 6 each used 3 μg of KP1.2-KLH and 3 μg KP5.1-KLH, adjuvanted with IFA, CpG and Alum as set out in Table 6. A seventh booster immunisation was added, which consisted of 2 μg of KP1.2-KLH and 2 μg KP5.1-KLH, adjuvanted with IFA, CpG and Alum as set out in Table 6. Antibodies were isolated from these mice. 17 antibodies were identified using B-cell cloning technique as described above with labelled KP1.2 peptide (SEQ ID No:10). These 17 antibodies were further tested for binding to human NAV1.7 peptide using a DELFIA assay.

Streptavidin Even Coat 96 well plates (R&D Systems) were coated with 50 μL of 4 μg/ml KP1.2 peptide (SEQ ID No:10, synthesised by Mimotopes) diluted in 1×PBS (Sigma) for 1 hr at room temperature (RT). Plates were then washed 3× with 1×PBS+0.1% tween (200 μL/well). Plates were blocked with 200 μL PBS+1% BSA (Sigma) for 1 hr at RT. Plates were then washed again 3× with 1×PBS+0.1% tween (Sigma) (200 μL/well). Purified antibodies were then added non-diluted to washed plate and incubated for 1 hr at RT (50 μL/well). Plates were then washed 3× with 1×PBS+0.1% tween (200 μL/well). 50 μL/well of Dissociation-Enhanced Lanthanide Fluorescent Immunoassay (DELFIA) Europium-labelled Anti-Human IgG antibody (Perkin Elmer) diluted 1:500 in DELFIA assay buffer (Perkin Elmer) for 1 hr at RT. Plates were then washed 3× with DELFIA wash buffer (Perkin Elmer) (200 μL/well). 50 μL/well of DELFIA Enhancement Solution (Perkin Elmer, warmed to RT before use) was added to each well and incubated for 5 min at RT on shaker (Heidolph Titramax). Plate was then read on Envision plate reader (Perkin Elmer) using filter-based time-resolved fluorescence (615 nM). Values were plotted using Prism (Graph Pad Prism).

12 out of the 17 tested antibodies were identified as binding to the recombinant expressed peptide of the D2E2 loop of Nav1.7 channel compared to the isotype control. The remaining antibodies, 27A03, 32A07, 35A10, 32E01 and 30G01 may be useful as negative controls for comparison in various assays as described herein.

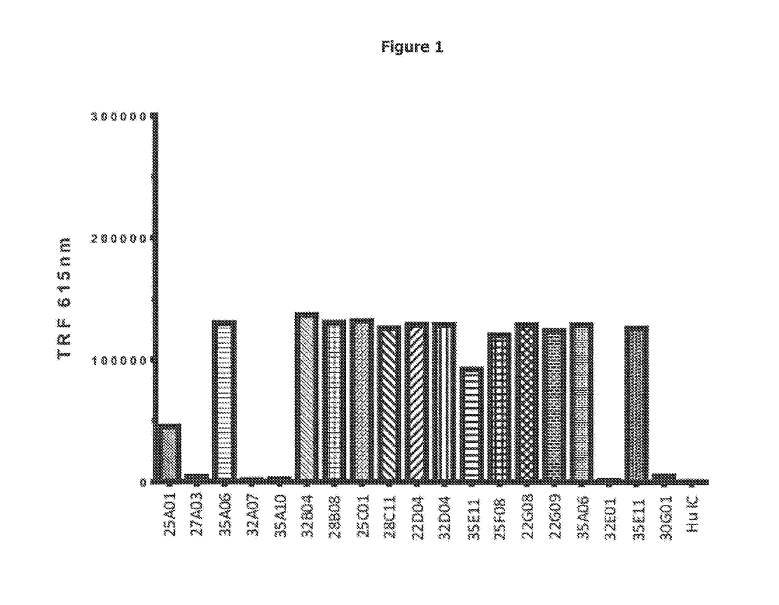

These data are depicted in FIG. 1.

### 2B: Antibody Binding to Cells Expressing NAV—Method 1

To further characterize anti-NAV antibodies, cells of the human embryonic kidney 293 cell line (HEK293) may be genetically engineered to overexpress full length NAV sequences (e.g. human NAV1.7, SEQ ID NO:2)

The binding of anti-NAV human antibodies to full-length NAV proteins expressed in HEK293 cells is determined by flow cytometry (FACS). HEK293 cells are stably-transfected with a full length NAV protein sequence fused at its N-terminus to the green fluorescent protein (GFP) to generate cell line NAV GFP-HEK293. Antibody binding to the transfected cells overexpressing the NAV proteins are compared to binding to the parental HEK293 cell line. To perform the binding experiments, adherent cells are collected using 1 mM EDTA in PBS, washed and re-suspended in cold PBS containing 5% FBS. For each binding experiment, the anti-NAV antibody (at concentrations ranging from 1 nM to 13 nM), was added to 250,000 cells in 500 μL of PBS with 5% FBS. After incubation for 20 minutes on ice, the secondary antibody, recognizing either human-Fc and conjugated to cyanine 5 (Cy5) or recognizing mouse-Fc and conjugated to allophyocyanin (APC), is then added to the cell mixture at a final secondary antibody concentration of 1.7 nM. After incubating for 20 minutes on ice, the cells are resuspended in PBS+5% FBS and then sorted and analyzed on a flow cytometer to determine relative binding by the candidate antibodies. For FACS analysis, gating is applied to examine only healthy live cells in the antibody binding experiments, and percentage stained was recorded. Specific binding is measured as the percent binding to NAV-GFP-HEK293 cells minus percent binding to parental HEK293 cells.

Cross reactivity may be measured by comparison of the binding to cells expressing the different NAV proteins of interest.

### 2C: Antibody Binding to Cells Expressing NAV—Method 2

In a similar experiment, anti-NAV antibodies may be tested for binding to a HEK293 cell that is genetically engineered to overexpress full length NAV proteins using an immunostaining procedure.

As in Example 2A, HEK293 cells are stably-transfected with full-length NAV protein fused at its N-terminus to a green fluorescent protein (GFP) to generate the cell line NAV-GFP-HEK293. Antibody binding to the transfected cells overexpressing the NAV protein are compared to binding to the parental HEK293 cell line. Cross-reactivity of anti-NAV protein antibodies are tested using HEK293 cells that express a different full length NAV protein after overnight induction with 1 μg/ml doxycycline.

Antibody binding to the transfected cells overexpressing the different NAV protein are compared to binding to the same HEK293 cell line without induction. Briefly, cells are plated onto POL coated 8 chambers culture slides at a density of 150,000 cells/well overnight at 37° C. The following day, media is removed and the cells are washed 3× with PBS. The cells are fixed with 4% PFA at RT for 20 minutes, then permeabilized with 0.05% Triton X-100 for 5 minutes at RT. The cells are blocked with superblock at RT for 1 hour, then incubated with 1 μg of anti-NAV antibodies at 4° overnight. The cells are then incubated in a 1:400 dilution of anti-human Alexa Fluor® 594 (Invitrogen) conjugated secondary antibody for 1 hr at RT and then imaged under a fluorescent microscope using a green filter.

### 2D: Antibody Binding to Cells Expressing NAV—Method 3

In another experiment, the anti-NAV antibodies may be tested for binding to full length NAV proteins expressed in the HEK293 cells that are genetically engineered to overexpress full length NAV proteins (as described in Example 2B and 2C) using a Western Blot procedure.

Cells are harvested using RIPA buffer supplemented with protease inhibitor. Fifteen microliters of cell lysate is combined with 15 μL of SDS sample buffer in a microfuge tube and the mixture is heated at 100° C. for 5 minutes. The samples are separated by SDS-PAGE and transferred to PVDF membrane. The membrane is blocked with 5% (w/v) non-fat dry milk for 1 hr at RT and then incubated with the anti-NAV antibody at 4° C. overnight. The membrane is washed with TBS-T 3×5 min and then incubated with secondary antibody (1:10,000 dilution) for 1 hr at RT. The secondary antibody is HRP-conjugated anti-human IgG or anti-mouse IgG according to the Fc fragment of the tested anti-NAV antibody. After washing the membrane 3×15 min, it is developed using Thermo Scientific ECL solution and exposed on light-sensitive film.

### 2E: Specificity of Anti-NAV Antibodies Binding to Cell-Surface NAV Proteins as Assessed by Peptide Binding Competition

The specificity of binding of anti-NAV antibodies to full-length NAV proteins expressed in HEK293 cells may be determined by peptide competition experiments using flow cytometry.

HEK293 cells stably-transfected with full-length NAV proteins fused at its N-terminus to GFP (NAV-GFP-HEK293) are used for this study. Antibody binding to the transfected cells overexpressing the NAV protein of interest is compared to binding in the presence of excess peptide and to binding to the parental HEK293 cell line. To perform the binding experiments, adherent cells are collected using 1 mM EDTA in PBS, washed and re-suspended in cold PBS containing 5% FBS. For each binding experiment, the anti-NAV antibody at a final concentration of 3.3 nM (in PBS/5% FBS) is added to cells either directly or after incubating for 10 min on ice with a 1000-fold molar excess (3.3 uM final concentration in PBS/5% FBS) of synthetic peptide with a sequence corresponding to the loop specificity of the antibody. The antibody or antibody-peptide mixture is then allowed to incubate with cells for 20 min on ice, followed by addition of 500 ul of cold PBS/5% FBS. Cells are collected by centrifugation, resuspended in 500 ul of cold PBS/5% FBS, and then secondary antibody matched to the antibody isotype (either anti-human-Fc conjugated to Cyanine 5 or anti-mouse Fc conjugated to allophyococyanin) was added. Binding is detected on the flow cytometry instrument and analyzed as percent binding using the instrument software. For FACS analysis, appropriate gating is applied so that only healthy, live cells are counted. Background staining of secondary antibodies alone to NAV-GFP-HEK293 cells are tested and recorded.

### 2F: Whole-Cell Patch-Clamp Recordings in HEK293 Cells

HEK293 cells are transfected with plasmids containing NaV channel cDNAs mixed with the plasmid containing GFP using lipofectamine 2000 (Invitrogen) at 1 mg of DNA per well of a 6-well plate. For NaV1.1 and NaV1.2, 1 mg of b1-subunit cDNA is cotransfected with the channel cDNAs. Approximately, 24 hr after transfection, whole-cell recordings are performed on single isolated green cell identified under a fluorescence microscope at RT. Glass pipettes are prepared (2-3 MU) using a vertical puller. Data are acquired with an Axopatch 200B amplifier controlled by Clampex 10 via a Digidata 1440A data acquisition system (Axon Instruments). Currents are sampled at a rate of 10 kHz and filtered at 3 kHz. The pipette solution contains (in mM): 10 NaCl, 110 CsCl, 20 TEA, 2.5 MgCl2, 5 EGTA, 3 ATP, 5 HEPES, pH 7.0 (adjusted with CsOH), and the osmolarity is adjusted to 300 mOsmol/L with glucose. The extracellular bath solution contains (in mM): 100 NaCl, 5 CsCl, 30 TEA, 1.8 CaCl₂, 1 MgCl₂, 0.1 CdCl₂, 5 HEPES, 25 Glucose, 5 4-aminopyridine, pH 7.4 (adjusted with CsOH), and the osmolarity is adjusted to 300 mOs-mol/L with glucose.

To record current-voltage relationships, after establishing the whole cell configuration, cells are held at −120 mV and current traces are elicited using 30 ms voltage steps between −80 and +60 mV with 10 mV increments. I-V curves are generated by plotting normalized peak currents (I/Imax) as a function of depolarization potential.

The voltage-dependence of channel activation is calculated by measuring the peak currents at test potentials ranging from −90 mV to +10 mV evoked in 5 mV increments from a holding potential of −120 mV. The conductance (GNa) is calculated according to the equation GNa=INa/(Vg−Vr), where INa is the peak amplitude of the Na+ current, Vg is the test potential, and Vr is the reversal potential for Na⁺. The conductance-voltage curves are drawn according to the equation GNa/maxGNa=1/{1+exp [(Vg0.5−Vg)/kg]}, where maxGNa is the maximum value for GNa, Vg0.5 is the potential at which GNa is 0.5 maxGNa, and kg is the slope factor (potential required for an e-fold change). The voltage-dependence of steady-state inactivation is determined using 500 ms conditioning pre-pulses ranging from −110 mV to −30 mV from a holding potential of −120 mV in 5 mV increments, followed by a test pulse to −10 mV for 30 ms. The peak INa is normalized to its respective maximum value (maxINa) and plotted as a function of the pre-pulse potential. The steady-state inactivation curves are drawn according to the equation INa/maxINa=1/{1+exp [(Vh−Vh0.5)/kh]} where Vh is the pre-pulse potential, Vh0.5 is the potential at which INa is 0.5 ₘₐₓI_{Na}, and kₕ is the slope factor. Data analysis and curve fitting are performed with OrignPro (OriginLab Corp).

### 2G: Whole-Cell Patch-Clamp Recordings in HEK293 Cells as Determined by IonWorks® Quattro (IWO) Patch Clamp Device

### Compound Handling

Samples are diluted in extracellular buffer containing 0.3% BSA and added to row H of a 96-well polypropylene micro-titre plate. A 3-fold up-plate serial dilution is performed. The standard Nay inhibitor tetracaine is included on each plate; 30 mM stock is diluted 1:100 in extracellular buffer to give a 300 pM solution and then an 8-point 1:3 serial dilution is performed. The IonWorks instrument performs a further 1:3 dilution by adding 3.5 μl to 7 μl in each assay well.

### IonWorks Electrophysiology

Electrophysiological recordings are made from a Human Embryonic Kidney (HEK293) cell line stably expressing a full length NAV protein of interest (e.g. a full length human Nav1.7 channel). Ionic currents are measured in the perforated patch clamp configuration (200 μg ml⁻¹ amphoterocin) at room temperature (21-23° C.) using an IonWorks Quattro instrument in population patch clamp (PPC) mode (Finkel et al, 2006, J Biomol Screen, 5:488). The internal solution contains (mM): 90 K gluconate, 40 KCl, 10 NaCl, 3.2 MgCl2, 3.2 EGTA, 5 HEPES and is buffered to pH 7.3. The external solution contain (mM): 137 NaCl, 4 KCl, 1.8 CaCl₂, 1 MgCl₂, 10 HEPES also buffered to pH 7.3. Cells are clamped at a holding potential of −90 mV for 30 s and then repeatedly stepped to 0 mV for 20 ms at a frequency of 10 Hz. Currents are measured from the 1^{st} and 25^{th} steps and referenced to the holding current. Compounds are then incubated for 5-7 min prior to a 2^{nd} measurement using an identical pulse train.

### Data Analysis

Data is analysed using IonWorks software v.2.0.4.4, Microsoft Excel (v7.0), XLFit (IDBS, v5.2.0.0) and GraphPad Prism (v5).

The following well QC criteria are employed. Cells failing these criteria are excluded from all subsequent analyses.

1. Seal resistance >20 MΩ on pre- and post-compound reads.

2. Peak Nav1.7 current amplitude >400 pA.

3. <150 pA change in baseline current across the 2.5 s post-compound read.

The following plate QC criteria are employed:

1. Z′ value>0.4 (Zhang et al, 1999, J Biomol Screen, 4:67).

2. Average seal resistance of >40 MΩ.

3. Average mean current amplitude of >0.5 nA.

After exclusion of wells that did not pass the QC criteria, the effects of compounds are quantified by dividing the NAV current amplitude in the presence of compound by the amplitude of the pre-compound NAV current, multiplied by 100. This % inhibition value is then normalised to the effect of the time/vehicle (low) control change, by dividing it by the average values for that test plate (see equation below). To ensure appropriate time matched controls, the vehicle controls for the corresponding half of the plate are used for normalisation. Sample or standard potency is determined by fitting a four parameter logistic equation to the responses across the concentration range, yielding an pIC₅₀/IC₅₀ value.

$$Norm . \% I = \frac{100 - ( ( post / pre ) \times 100 )}{Average ( ( VEHICLEpost / VEHICLEpre ) \times 100 )}$$

### 2H: Whole-Cell Patch-Clamp Recordings in HEK293 Cells as Determined by Port-a-Patch® Patch Clamp Device

A microchip-based patch-clamp system (Port-a-Patch®, Nanion Technologies, Munich, Germany) may be used to determine the ability of anti-NAV protein antibodies to inhibit voltage gated ion flux through the NAV ion channels. For these experiments, a HEK293 cell line stably transfected with a NAV protein of interest fused at its N-terminus to GFP is used (NAV-GFP-HEK293).

On the day of the recording, cells are harvested by treatment with trypsin (0.025%), centrifuged and resuspended in 500 ul of the extracellular buffer solution (140 mM NaCl, 4 mM KCl, 1 mM MgCl₂, 2 mM CaCl₂, 5 mM glucose and 10 mM HEPES, adjusted to pH 7.3 with NaOH). Five microliters of the cell suspension are then loaded onto the recording chip. Cells are first perfused with the extracellular buffer solution containing 0.1% (w/v) bovine serum albumin for about 5 minutes to stabilize the patch. The NAV current is elicited with a repetitive depolarizing step to 0 mV for 20 ms from a holding potential of −100 mV, every 10 s. Cells exhibiting a sodium inward current greater than 1 nA are tested for ion-channel inhibition by addition of test or control antibody solutions at 300, 50 or 30 nM final concentration. The composition of the intracellular recording solution is 140 mM CsF, 10 mM NaCl, 1 mM EGTA and 10 mM HEPES, adjusted to pH 7.3 with CsOH. Tetrodotoxin, a well-validated voltage-gated sodium channel blocker is applied at the end of the experiment as a positive control.

Channel blocking is measured as percent inhibition of observed current flux in the presence of antibody relative to current flux in the absence of antibody, averaged over multiple blocking experiments.

### 2I: Whole-Cell Patch-Clamp Recordings in HEK293 Cells as Determined by Q-Patch Clamp Device

A NAV protein stable cell line or a stably-transfected HEK293 cell line expressing full length NAV receptor, plus a GFP tag at the N-terminus, may be used to test the effect of the anti-NAV protein antibodies on NAV protein current using the Q-Patch (Sophion Biosciences) automated patch clamp platform.

On the day of the recordings, cells are harvested with accutase cell detachment solution (Millipore, cat# SRC005) and resuspended in 1 ml of a serum free solution [CHO-SFM-11 media (Invitrogen, #31033), HEPES 25 mM and penicillin/streptomycin 100 units/ml]. The cell suspension is left on a shaker at RT for 30 minutes before they are loaded on the Q-Patch. NAV protein current is elicited by one depolarizing pulse to −30 mV for 20 ms followed by a depolarizing pulse to 0 mV for 20 ms (5 seconds apart) from a holding potential of −100 mV, every 30 seconds. Anti-NAV protein antibodies are diluted to a final concentration of 100 nM in the extracellular buffer (137 mM NaCl, 4 mM KCl, 1.8 mM CaCl₂, 1 mM MgCl2, 10 mM Glucose, 10 mM HEPES, pH=7.3) containing 0.2% Bovine Serum Albumin (BSA). TTX (100 nM) is included at the end of the experiment as a positive control.

Cells are first incubated with 0.2% BSA for 16 minutes with repetitive pulsing to allow stabilization of the current and with the anti-NAV protein antibodies in the presence of 0.2% BSA for another 16 minutes with repetitive pulsing. The voltage-dependence of the current is recorded at the end of the incubation with 0.2% BSA and at the end of the incubation with the antibody, currents are elicited with step depolarization from −85 mV to +30 mV in 5 my increment from a holding potential of −100 mV. All antibodies are tested at least in duplicate on three or more separate days. Channel blocking is measured as percent inhibition of observed current flux in the presence of antibody and 0.2% BSA relative to current flux in the absence of antibody, averaged over multiple blocking experiments.

### 2J: Whole-Cell Patch-Clamp Recordings in Dissociated DRG Neurons and Whole-Mount DRG

The dissociated DRGs are removed aseptically from mice (4-6 weeks) and incubated with collagenase (1.25 mg/ml, Roche)/dispase-II (2.4 units/ml, Roche) at 37° C. for 90 min, then digested with 0.25% trypsin for 8 min at 37° C., followed by 0.25% trypsin inhibitor. Cells are mechanically dissociated with a flame polished Pasteur pipette in the presence of 0.05% DNase I (Sigma). DRG cells are plated on glass coverslips and grown in a neurobasal defined medium (with 2% B27 supplement, Invitrogen) with 5 mM AraC and 5% carbon dioxide at 36.5° C. DRG neurons are grown for 24 hr before use.

The L4-L5 whole mount DRGs are carefully removed from the vertebral column and placed in cold oxygenated ACSF. The connective tissue is gently removed under a microscope and the ganglia were digested with a mixture of 1.0 mg/ml protease and 1.6 mg/ml collagenase (Sigma) for 30 min at 37° C. The ganglion is transferred into a holding chamber containing normal Mg²⁺-free ACSF with CNQX (2 pM) bubbled with 95% 02 and 5% CO₂ at room temperature.

Whole-cell voltage and current clamp recordings are performed at room temperature to measure transient and persistent sodium currents and action potentials, respectively, with Axopatch-200B amplifier (Axon Instruments) and Digidata 1440A data acquisition system (Axon Instruments). The patch pipettes are pulled from borosilicate capillaries (Chase Scientific Glass Inc.). When filled with the pipette solution, the resistance of the pipettes was 4-5 MΩ. The recording chamber (300 ml) is continuously superfused (3-4 ml/min). Series resistance is compensated for (>80%), and leak subtraction is performed. Data are lowpass-filtered at 2 KHz, sampled at 10 KHz. The pClamp10 (Axon Instruments) software is used during experiments and analysis.

For sodium current recording, pipette solution contains (in mM): CsCl 100, sodium L-glutamic acid 5, TEACl 30, CaCl₂ 0.1, MgCl₂ 2, EGTA 11, HEPES 10, adjusted to pH 7.4 with CsOH. The external solution is composed of (in mM): NaCl 90, choline chloride 30, TEACl 20, CaCl₂ 0.1, MgCl₂ 5, CoCl₂ 5, HEPES 10, glucose 10 adjusted to pH 7.4 with NaOH. In voltage-clamp experiments, the transient sodium current (INa) is evoked by a test pulse to +0 mV from the holding potential, −70 mV. The persistent sodium current (INaP) is recorded by applying a 3 s depolarization ramp current from −80 to −10 mV at a holding potential of −60 mV. The plot is fitted using the Origin software (Origin, Northampton, Mass., USA). The pipette solution for current-clamp experiments is composed of (in mM): K-gluconate 145, MgCl₂ 2, CaCl₂ 1, EGTA 10, HEPES 5, K₂ATP 5, adjusted to pH 7.4 with KOH. The external solution contains (in mM): NaCl 140, KCl 5, MgCl₂ 1, CaCl₂ 2, HEPES 10, glucose 10, adjusted to pH 7.4 with NaOH. In current-clamp experiments, action potentials are recorded under current clamp (−60 mV), with 1 s depolarizing current pulses with 200 pA amplitude.

### Example 3: In Vivo Characterisation of Antibodies

### 3A: Itch Models and Behavioural Testing of Itch

Mice are habituated to the testing environment daily for at least two days before analysis. Mice are shaved at the back of the neck the day before injection. Mice are left in small plastic chambers on an elevated metal mesh floor and allowed 30 min for habituation before examination. To elicit acute itch, 50 ml of pruritic agent compound 48/80 (100 mg) or chloroquine (200 mg, Sigma Aldrich) is injected intradermally in the nape of the neck or GRP (1 nmol) intrathecally and the number of scratches are counted every 5 min for 30 min after the injection. A scratch is counted when a mouse lifts its hindpaw to scratch the shaved region and returns the paw to the floor or to the mouth for licking.

To induce chronic itch, the neck skin is painted with acetone and diethyether (1:1) following by water (AEW) twice a day for 4 days and spontaneous itch is examined by counting the number of scratches for 60 min on day 5. To determine chronic itch-induced synaptic plasticity in the lumbar superficial spinal cord, the hindpaw is painted with AEW. The allergic contact dermatitis (ACD) model of chronic itch is generated by applying the hapten 2,4-dinitrofluorobenzene (DNFB) to the back skin. DNFB is dissolved in a mixture of acetone:olive oil (4:1) for sensitization and challenge. One day before sensitization, the surface of abdomen and the nape of neck is shaved. Mice are sensitized with 0.5% DNFB solution (50 ml) by topical application to a ˜2 cm² area of shaved abdomen skin. Five days later, mice are challenged with 0.2% DNFB solution (30 ml) by painting the shaved neck area, then every other day for one week. Spontaneous scratching behaviours are videoed for 1 hr, at 24 hr after each challenge. The behavioral tests of itch are performed blindly.

### 3B: Effect of Anti-NNAV Protein Antibodies In Vivo in a Rat Model of Acute Nociception

A study is conducted to evaluate the effects of certain of the anti-NAV protein antibodies in rats to determine their effect on acute nociception. Anti-NAV protein antibodies, as well as a control isotype-matched antibody are administered via intraperitoneal injection at a dose of 50 mg/kg.

Mechanical and thermal nociceptive threshold (paw pressure, and Hargreaves' test, respectively) testing is conducted before antibody administration and again approximately 24 and 48 hours after injection of the antibodies.

Male Sprague-Dawley rats are housed 3 per cage, acclimated to the facility prior to study initiation and dosed in a fed state. All testing is done in a blinded manner.

After the pre-treatment baseline assessment, animals are assigned to treatment groups based on baseline response thresholds for the paw pressure endpoint, so that group means are approximately equal. Briefly, all animals that met the inclusion criteria above are ranked by response threshold from lowest to highest and treatments were assigned as follows (e.g. A, B, C, D, E, B, C, D, E, A, C, D, E, A, B, D, E, A, B, C, etc). The animals are then dosed in sequence, based on treatment time, so that the distribution of treatment across a given set of animals is not predictable.

Antibodies are prepared individually by dilution with Phosphate Buffered Saline (Sigma Phosphate Buffered Saline 10× Concentrate, diluted 1:9 vol:vol with saline solution 0.9%). Briefly, antibodies stored frozen are allowed to reach room temperature, and then adjusted to a concentration of 50 mg/ml from the pre-labelled concentration. All antibodies are administered via intraperitoneal injection in a dose volume of 1 ml/kg based on individual animal body weight.

### Mechanical Threshold Testing

Baseline and post-treatment paw withdrawal thresholds to a mechanical stimulus are measured using the Randall-Selitto paw pressure apparatus (Ugo Basile Analgesymeter). This apparatus generates a linearly increasing mechanical force. The stimulus is applied to the plantar surface of the hind paw by a dome-shaped plastic tip placed between the 3rd and 4th metatarsus. To avoid tissue damage, a cutoff pressure is set at 250 g. Mechanical thresholds are defined as the force in grams at the first pain behavior, which includes paw withdrawal, struggle, and/or vocalization. The mean and standard error of the mean (SEM) are determined for the injured paws for each treatment group.

### Thermal Threshold Testing

Baseline and post-treatment paw withdrawal latencies to a noxious thermal stimulus are measured using the radiant heat test (Hargreaves, K. et al., 1988, Pain, Vol. 32(1):77-88) using a plantar test apparatus. The stimulus intensity is set to 30% of maximum output and the cut-off time is set at 45 seconds. Rats are placed on a glass platform warmed to 28±2° C. and allowed to habituate to the testing chambers for a minimum of 15 minutes prior to each test session. The thermal stimulus is applied to the plantar surface of the paw, and three readings per rat per paw are taken at each test session. Thermal thresholds are defined as the latency in seconds to the first pain behavior, which includes nocifensive paw withdrawal, flinching, biting and/or licking of the stimulated paw. Three readings for each paw per animal are averaged at each individual time point, and the mean and standard error of the mean (SEM) are determined for the left and right paws (pooled values) for each treatment group.

To determine whether the test articles significantly alter paw withdrawal thresholds or thermal nociceptive responses, an unpaired t-test is run at each time point (1, 2, and 4 hours post-treatment) comparing a control antibody, with the given candidate antibodies. Statistical analyses are conducted using Prism™ 5.01.

### 3C: Effect of Anti-Nav1.7 Antibodies on Reduction of Pain In Vivo in a Carageenan Pain Model

Injection of A-carrageenan, a polysaccharide obtained from seaweed extract, produces robust inflammation and nociceptive hypersensitivity with a peak effect at 3-5 hours post-injection. Anti-NAV protein antibodies may be tested for the ability to decrease A-carrageenan-induced thermal nociceptive hypersensitivity.

C57BL/6 mice are separated into groups of eight mice per antibody tested. All mice receive a dose of about 50 mg/kg of antibody by subcutaneous injection. A control group of C57BL/6 mice receive an irrelevant antibody of the same isotype as the test antibodies.

Peripheral inflammation is produced in the mice by a 25 μL subcutaneous (s.c.) injection of a 1%-2% λ-carrageenan solution (dissolved in saline) into the subplantar side of the left hind paw. The hind paw thermal sensitivity of the mice before and at 1 and 3 hours after λ-carrageenan injection is tested using the Hargreaves' apparatus, which measures the latency of the animals to withdraw their hind paw from a noxious thermal stimulus.

Three separate measurements are performed for each mouse and the mean thermal nociception threshold for each group is calculated (mean±SEM). The mean values for each group are statistically compared to the control group using a one-way analysis of variance (ANOVA). The amount of edema present is also determined by measuring hindpaw thickness with calipers before and at 3 hours after λ-carrageenan injection. Blood is collected at the end of the experiment and the levels of circulating anti-NAV protein antibodies (serum Ab) are determined using a standard ELISA assay. Briefly, plates are coated with a goat anti-human Fc antibody to capture serum Ab. Serum is then added to the plates, and captured anti-NAV protein antibodies are detected by colorimetric substrate using a horseradish peroxidase (HRP) conjugated goat anti-human IgG antibody.

The animals receiving an effective dose of an anti-NAV protein antibody sufficient to block or neutralize NAV protein activity will demonstrate a significant reduction in thermal sensitivity as compared to animals receiving an irrelevant antibody of the same isotype.

### 3D: Inflammatory and Neuropathic Pain Models in Mice

To produce inflammatory pain, diluted formalin (5%, 20 ml) is injected into the plantar surface of a hindpaw. Neuropathic pain is produced by chronic constriction injury (CCI) of the sciatic nerve. Mice are anesthetized with isoflurane, and three ligatures with 7-0 prolene are placed around the nerve proximal to the trifurcation (1 mm between ligatures). The ligatures are loosely tied until a short flick of the ipsilateral hind limb was observed.

Animals are habituated to the environment for at least 2 days before the testing. All the behaviours are tested blindly. Formalin-evoked spontaneous inflammatory pain is investigated by measuring the time (seconds) mice spent on licking or flinching the affected paw every 5 min for 45 min. For testing mechanical sensitivity after nerve injury, mice are confined in boxes placed on an elevated metal mesh floor and their hindpaws are stimulated with a series of von Frey hairs with logarithmically increasing stiffness (0.02-2.56 g, Stoelting), presented perpendicularly to the central plantar surface. The 50% paw withdrawal threshold is determined by Dixon's up-down method (Dixon, 1980). For testing motor function, a rota-rod system is used. Mice are tested for three trails separated by 10 min intervals and during the tests, the speed of rotation is accelerated from 2 to 20 r.p.m. in 3 min, and the falling latency is recorded (Liu et al., 2012). The behavioral tests of pain are performed blindly.

| SEQ | | |
|---|---|---|
| ID No | Sequence | Description |
| 1 | ATGGCTATGCTGCCTCCACCTGGCCCTCAGAGCTTCGTGCACTTCACC | hNAv1.7-nucleotide |
| | AAGCAGAGCCTGGCCCTGATCGAGCAGAGAATCGCCGAGAGAAAGAG | (codon optimised) |
| | CAAAGAGCCCAAAGAGGAAAAGAAGGACGACGACGAGGAAGCCCCCAA | |
| | GCCCAGCAGCGATCTGGAAGCTGGAAAGCAGCTGCCCTTCATCTACGG | |
| | CGACATCCCCCCTGGCATGGTGTCCGAGCCTCTGGAAGATCTGGACCC | |
| | CTACTACGCCGACAAGAAAACCTTCATCGTGCTGAACAAGGGCAAGAC | |
| | CATCTTCAGGTTCAACGCCACCCCTGCCCTGTACATGCTGAGCCCCTT | |
| | CAGCCCCCTGAGAAGAATCAGCATCAAGATCCTGGTGCACAGCCTGTT | |
| | CTCCATGCTGATCATGTGCACCATCCTGACCAACTGCATCTTCATGACC | |
| | ATGAACAACCCCCCCGACTGGACCAAGAACGTGGAGTACACCTTCACC | |
| | GGCATCTACACCTTCGAGAGCCTCGTGAAGATTCTGGCCAGGGGCTTC | |
| | TGCGTGGGCGAGTTCACATTCCTGAGGGACCCCTGGAACTGGCTGGA | |
| | CTTCGTCGTGATCGTGTTCGCCTACCTGACCGAGTTCGTGAACCTGGG | |
| | CAACGTGTCCGCCCTGAGAACCTTCAGAGTGCTGAGAGCCCTGAAAAC | |
| | CATCAGCGTGATCCCCGGCCTGAAAACAATCGTGGGCGCCCTGATCCA | |
| | GAGCGTGAAGAAACTGAGCGACGTGATGATCCTGACCGTGTTCTGCCT | |
| | GTCTGTGTTCGCTCTGATCGGCCTGCAGCTGTTCATGGGCAACCTGAA | |
| | GCACAAGTGCTTCCGGAACAGCCTGGAAAACAACGAGACACTGGAATC | |
| | CATCATGAACACCCTGGAATCCGAAGAGGATTTCCGCAAGTACTTCTA | |
| | CTACCTGGAAGGCAGCAAGGACGCCCTGCTGTGCGGCTTCTCTACAGA | |
| | CAGCGGCCAGTGCCCCGAGGGCTACACCTGTGTGAAGATCGGCAGAA | |
| | ACCCCGACTACGGCTACACCAGCTTCGATACCTTCAGCTGGGCCTTCC | |
| | TGGCTCTGTTCAGACTGATGACCCAGGACTACTGGGAGAACCTGTACC | |
| | AGCAGACCCTGAGAGCCGCTGGCAAGACCTACATGATCTTTTTCGTGG | |
| | TCGTGATCTTCCTGGGCAGCTTCTACCTGATCAACCTGATCCTGGCTG | |
| | TGGTGGCCATGGCTTACGAGGAACAGAACCAGGCCAACATCGAAGAG | |
| | GCCAAGCAGAAAGAGCTGGAATTTCAGCAGATGCTGGACCGGCTGAA | |
| | GAAAGAACAGGAAGAGGCCGAGGCCATTGCCGCCGCTGCTGCCGAGT | |
| | ACACATCCATCAGGCGGAGCAGAATCATGGGCCTGAGCGAGAGCAGCA | |
| | GCGAGACAAGCAAGCTGAGCAGCAAGTCCGCCAAAGAGAGAAGAAACC | |
| | GGCGCAAGAAGAAGAACCAGAAGAAGCTGTCCAGCGGCGAGGAAAAG | |
| | GGCGACGCCGAGAAACTGTCCAAGAGCGAGTCCGAGGACAGCATCAG | |
| | AAGAAAGTCCTTCCACCTGGGCGTGGAAGGCCACAGAAGGGCCCACGA | |
| | GAAGAGACTGAGCACCCCCAACCAGAGCCCTCTGAGCATCAGGGGCAG | |
| | CCTGTTTAGCGCCAGAAGATCCAGCAGAACCTCCCTGTTCAGCTTCAA | |
| | GGGCAGAGGCAGAGACATCGGCTCCGAGACAGAGTTCGCCGACGATG | |
| | AGCACAGCATCTTCGGCGATAACGAGAGCAGACGGGGCTCTCTGTTCG | |
| | TGCCCCACAGACCCCAGGAAAGAAGAAGCAGCAACATCAGCCAGGCCA | |
| | GCAGATCCCCCCCCATGCTGCCTGTGAACGGCAAGATGCACAGCGCCG | |
| | TGGACTGCAACGGCGTGGTGTCTCTGGTGGATGGCAGATCCGCACTG | |
| | ATGCTGCCCAACGGCCAGCTGCTGCCTGAGGGCACAACAAATCAAATA | |
| | CACAAGAAAAGGCGTTGTAGTTCCTATCTCCTTTCAGAGGATATGCTG | |
| | AACGACCCCAACCTGAGACAGAGAGCCATGAGCAGAGCCAGCATCCTG | |
| | ACCAACACCGTGGAAGAACTGGAAGAGTCCAGACAGAAATGCCCCCCC | |
| | TGGTGGTACAGATTCGCCCACAAGTTTCTGATCTGGAACTGCAGCCCC | |
| | TACTGGATCAAGTTCAAGAAGTGCATCTACTTCATCGTGATGGACCCC | |
| | TTCGTGGACCTGGCCATCACCATCTGCATCGTGCTGAACACCCTGTTC | |
| | ATGGCTATGGAACACCACCCCATGACCGAGGAATTCAAGAACGTGCTG | |
| | GCCATCGGCAACCTGGTGTTCACCGGCATCTTCGCCGCCGAGATGGTG | |
| | CTGAAGCTGATCGCCATGGACCCTTACGAGTACTTCCAAGTGGGCTGG | |
| | AACATCTTCGACAGCCTGATCGTGACCCTGAGCCTGGTGGAACTGTTC | |
| | CTGGCCGACGTGGAAGGCCTGAGCGTGCTGAGAAGCTTCAGACTGCT | |
| | GAGAGTGTTCAAGCTGGCCAAGAGCTGGCCCACCCTGAACATGCTGAT | |
| | CAAGATCATCGGAAACAGCGTGGGCGCCCTGGGCAACCTGACACTGGT | |
| | GCTGGCTATCATCGTGTTCATCTTCGCTGTCGTGGGCATGCAGCTGTT | |
| | CGGCAAGAGCTACAAAGAATGCGTGTGCAAGATCAACGACGACTGCAC | |
| | CCTGCCCAGATGGCACATGAACGATTTCTTCCACAGCTTTCTGATCGT | |
| | GTTCCGGGTGCTGTGCGGCGAGTGGATCGAGACAATGTGGGACTGCA | |
| | TGGAAGTGGCTGGCCAGGCCATGTGCCTGATTGTGTACATGATGGTCA | |
| | TGGTCATCGGGAATCTGGTGGTGCTGAACCTGTTTCTGGCCCTGCTGC | |
| | TGTCCAGCTTCTCCAGCGATAACCTGACCGCCATCGAAGAGGACCCCG | |
| | ACGCCAACAACCTGCAGATCGCCGTGACCAGAATCAAGAAAGGCATCA | |
| | ACTACGTGAAGCAGACCCTGCGCGAGTTCATCCTGAAGGCTTTCAGCA | |
| | AGAAGCCCAAGATCAGCAGAGAGATCAGACAGGCCGAGGACCTGAACA | |
| | CCAAGAAAGAGAACTACATCAGCAACCACACCCTGGCCGAGATGAGCA | |
| | AGGGCCATAACTTTCTGAAAGAGAAGGACAAGATCTCCGGCTTCGGCA | |
| | GCAGCGTGGACAAGCACCTGATGGAAGATAGCGACGGCCAGAGCTTC | |
| | ATCCACAACCCCTCCCTGACCGTGACCGTGCCTATCGCTCCTGGCGAG | |
| | AGCGACCTGGAAAACATGAACGCCGAGGAACTGAGCAGCGACAGCGA | |
| | CTCCGAGTACAGCAAAGTGCGGCTGAACAGATCCAGCAGCAGCGAGTG | |
| | CTCCACCGTGGACAATCCTCTGCCAGGCGAGGGCGAGGAAGCTGAGG | |
| | CTGAGCCTATGAACAGCGACGAGCCCGAGGCCTGTTTCACCGATGGCT | |
| | GCGTGCGGAGATTCAGCTGCTGCCAAGTGAACATCGAGAGCGGCAAG | |
| | GGCAAGATCTGGTGGAATATCAGAAAGACCTGCTACAAGATCGTGGAA | |
| | CACAGTTGGTTCGAGAGCTTTATTGTGCTGATGATCCTGCTGTCCTCC | |
| | GGCGCTCTGGCCTTCGAGGACATCTACATCGAGCGGAAGAAAACCATC | |
| | AAGATTATCCTGGAATACGCCGATAAGATCTTCACCTACATCTTCATCC | |
| | TGGAAATGCTGCTGAAGTGGATCGCTTACGGCTACAAGACCTACTTCA | |
| | CCAACGCCTGGTGTTGGCTGGACTTTCTGATTGTGGACGTGTCCCTCG | |
| | TGACTCTGGTGGCCAACACCCTGGGCTACAGCGATCTGGGCCCCATCA | |
| | AGAGCCTGAGAACCCTGAGGGCTCTGAGGCCACTGAGAGCCCTGTCCA | |
| | GATTCGAGGGCATGAGAGTGGTTGTGAATGCACTCATAGGAGCAATTC | |
| | CTTCCATCATGAATGTGCTACTTGTGTGTCTGATCTTCTGGCTGATCTT | |
| | CAGCATCATGGGCGTGAACCTGTTCGCCGGCAAGTTCTACGAGTGCAT | |
| | CAACACCACCGACGGCAGCAGATTCCCCGCCAGCCAGGTGCCAAACAG | |
| | ATCCGAGTGCTTCGCCCTGATGAACGTGTCCCAGAACGTGCGGTGGAA | |
| | GAACCTGAAAGTGAACTTCGACAACGTGGGCCTGGGCTACCTGAGCCT | |
| | GCTGCAGGTGGCCACATTCAAGGGCTGGACCATCATTATGTACGCCGC | |
| | CGTGGACAGCGTGAACGTGGACAAGCAGCCTAAGTACGAGTACAGCCT | |
| | GTACATGTATATCTACTTCGTGGTGTTTATTATCTTCGGCAGCTTCTTC | |
| | ACCCTGAATCTGTTCATCGGCGTGATCATCGACAACTTCAACCAGCAG | |
| | AGAAGAAACTGGGCGGCCAGGACATCTTCATGACCGAGGAACAGAAA | |
| | AAGTACTACAACGCCATGAAGAAGCTGGGCAGCAAGAAGCCCCAGAAG | |
| | CCCATCCCCAGACCCGGCAACAAGATCCAGGGCTGCATCTTCGACCTC | |
| | GTGACCAACCAGGCCTTCGACATCTCCATCATGGTGCTGATCTGCCTG | |
| | AACATGGTCACAATGATGGTGGAAAAAGAGGGCCAGAGCCAGCACATG | |
| | ACAGAGGTGCTGTACTGGATCAACGTGGTGTTCATCATCCTGTTCACC | |
| | GGCGAGTGCGTGCTGAAGCTGATCTCCCTGCGGCACTACTACTTCACC | |
| | GTGGGCTGGAACATCTTCGATTTCGTGGTCGTGATCATTTCTATCGTG | |
| | GGCATGTTCCTGGCCGACCTGATCGAGACATACTTCGTGTCCCCCACC | |
| | CTGTTCAGAGTGATCAGACTGGCCAGAATCGGCAGAATCCTGAGACTC | |
| | GTGAAGGGCGCCAAGGGCATCAGAACCCTGCTGTTCGCTCTGATGATG | |
| | AGCCTGCCCGCCCTGTTCAATATCGGCCTGCTGCTGTTCCTCGTGATG | |
| | TTCATCTACGCCATCTTCGGGATGAGCAACTTCGCCTACGTGAAGAAA | |
| | AGGACGGCATCAACGACATGTTCAACTTCGAGACATTCGGCAACAGC | |
| | ATGATCTGTCTGTTCCAGATCACCACCAGCGCCGGCTGGGATGGACTG | |
| | CTGGCTCCTATCCTGAACAGCAAGCCCCCCGACTGCGACCCCAAGAAG | |
| | GTGCACCCTGGCAGCAGCGTGGAAGGCGACTGTGGCAACCCTAGCGT | |
| | GGGCATCTTCTACTTTGTGTCCTATATCATCATTAGCTTTCTGGTGGTC | |
| | GTGAACATGTACATTGCCGTGATCCTGGAAAACTTCAGCGTGGCCACC | |
| | GAGGAAAGCACCGAGCCTCTGAGCGAGGACGACTTCGAGATGTTCTAC | |
| | GAAGTGTGGGAGAAGTTCGACCCCGACGCCACCCAGTTCATCGAGTTC | |
| | AGCAAGCTGAGCGACTTCGCTGCCGCCCTGGACCCTCCTCTGCTGATC | |
| | GCCAAGCCTAACAAGGTGCAGCTGATCGCTATGGACCTGCCCATGGTG | |
| | TCCGGCGACAGAATCCACTGCCTGGACATCCTGTTTGCCTTCACCAAG | |
| | AGAGTGCTGGGCGAGAGCGGCGAGATGGACAGCCTGAGAAGCCAGAT | |
| | GGAAGAAAGATTCATGAGCGCCAACCCCAGCAAGGTGTCCTACGAGCC | |
| | CATCACCACAACCCTGAAGAGAAAGCAGGAAGATGTGTCCGCCACCGT | |
| | GATCCAGAGAGCCTACAGAAGATACAGGCTGAGGCAGAATGTGAAGAA | |
| | CATCAGCAGCATCTACATCAAGGACGGCGACAGGGACGACGACCTGCT | |
| | GAACAAGAAAGACATGGCCTTCGATAACGTGAACGAGAACAGCTCCCC | |
| | CGAAAAGACAGACGCCACCAGCAGCACCACCTCCCCACCTAGCTACGA | |
| | CTCCGTGACCAAGCCCGACAAAGAGAAGTACGAGCAGGACAGAACCGA | |
| | GAAAGAAGATAAGGGCAAGGACAGCAAAGAAAGCAAGAAGTGA | |
| 2 | MAMLPPPGPQSFVHFTKQSLALIEQRIAERKSKEPKEEKKDDDEEAPKPS | hNAv1.7-amino acid |
| | SDLEAGKQLPFIYGDIPPGMVSEPLEDLDPYYADKKTFIVLNKGKTIFRFN | NP_002968 |
| | ATPALYMLSPFSPLRRISIKILVHSLFSMLIMCTILTNCIFMTMNNPPDVVT | |
| | KNVEYTFTGIYTFESLVKILARGFCVGEFTFLRDPWNWLDFVVIVFAYLTE | |
| | FVNLGNVSALRTFRVLRALKTISVIPGLKTIVGALIQSVKKLSDVMILTVFC | |
| | LSVFALIGLQLFMGNLKHKCFRNSLENNETLESIMNTLESEEDFRKYFYYL | |
| | EGSKDALLCGFSTDSGQCPEGYTCVKIGRNPDYGYTSFDTFSWAFLALFR | |
| | LMTQDYWENLYQQTLRAAGKTYMIFFVVVIFLGSFYLINLILAVVAMAYE | |
| | EQNQANIEEAKQKE1EFQQMLDRLKKEQEEAEAIAAAAAEYTSIRRSRIM | |
| | GLSESSSETSKLSSKSAKERRNRRKKKNQKKLSSGEEKGDAEKLSKSESE | |
| | DSIRRKSFHLGVEGHRRAHEKRLSTPNQSPLSIRGSLFSARRSSRTSLFSF | |
| | KGRGRDIGSETEFADDEHSIFGDNESRRGSLFVPHRPQERRSSNISQASR | |
| | SPPMLPVNGKMHSAVDCNGVVSLVDGRSALMLPNGQLLPEGTTNQIHKK | |
| | RRCSSYLLSEDMLNDPNLRQRAMSRASILTNTVEEIEESRQKCPPWWYR | |
| | FAHKFLIWNCSPYWIKFKKCIYFIVMDPFVDLATTICIVLNTLFMAMEHHP | |
| | MTEEFKNVLAIGNLVFTGIFAAEMVLKLIAMDPYEYFQVGWNIFDSLIVTL | |
| | SLVEIFLADVEGLSVLRSFRLLRVFKLAKSWPTLNMLIKIIGNSVGALGNLT | |
| | LVLAIIVFIFAVVGMQLFGKSYKECVCKINDDCTLPRWHMNDFFHSFLIVF | |
| | RVLCGEWIETMWDCMEVAGQAMCLIVYMMVMVIGNLVVLNLFLALLLSS | |
| | FSSDNLTAIEEDPDANNLQIAVTRIKKGINYVKQTLREFILKAFSKKPKISR | |
| | EIRQAEDLNTKKENYISNHTLAEMSKGHNFLKEKDKISGFGSSVDKHLME | |
| | DSDGQSFIHNPSLTVTVPIAPGESDLENMNAEE1SSDSDSEYSKVRLNRS | |
| | SSSECSTVDNPLPGEGEEAEAEPMNSDEPEACFTDGCVRRFSCCQVNIES | |
| | GKGKIWWNIRKTCYKIVEHSWFESFIVLMILLSSGALAFEDIYIERKKTIKI | |
| | ILEYADKIFTYIFILEMLLKWIAYGYKTYFTNAWCWLDFLIVDVSLVTLVA | |
| | NTLGYSDLGPIKSLRTLRALRPLRALSRFEGMRVVVNALIGAIPSIMNVLL | |
| | VCLIFWLIFSIMGVNLFAGKFYECINTTDGSRFPASQVPNRSECFALMNVS | |
| | QNVRWKNLIWNFDNVGLGYLSLLQVATFKGWTIIMYAAVDSVNVDKQP | |
| | KYEYSLYMYIYFVVFIIFGSFFTLNLFIGVIIDNFNQQKKKLGGQDIFMTEE | |
| | QKKYYNAMKKLGSKKPQKPIPRPGNKIQGCIFDLVINQAFDISIMVLICLN | |
| | MVTMMVEKEGQSQHMTEVLYVVINVVFIILFTGECVLKLISLRHYYFTVG | |
| | WNIFDFVVVIISIVGMFLADLIETYFVSPTLFRVIRLARIGRILRLVKGAKGI | |
| | RTLLFALMMSLPALFNIGLLLFLVMFIYAIFGMSNFAYVKKEDGINDMFNF | |
| | ETFGNSMICLFQTTTSAGWDGLLAPILNSKPPDCDPKKVHPGSSVEGDCG | |
| | NPSVGIFYFVSYIIISFLVVVNMYIAVILENFSVATEESTEPLSEDDFEMFYE | |
| | VWEKFDPDATQFIEFSKLSDFAAALDPPLLIAKPNKVQLIAMDLPMVSGD | |
| | RIHCLDILFAFTKRVLGESGEMDSLRSQMEERFMSANPSKVSYEPITTTLK | |
| | RKQEDVSATVIQRAYRRYRLRQNVKNISSIYIKDGDRDDDLLNKKDMAF | |
| | DNVNENSSPEKTDATSSTTSPPSYDSVTKPDKEKYEQDRTEKEDKGKDS | |
| | KESKK | |
| 3 | FMTMNNPP | hNAv1.7D1 E1 loop |
| 4 | LTEFVNLGN | hNAv1.7D1 E2 loop |
| 5 | MGNLKHKCFRNSLENNETLESIMNTLESEEDFRKYFYYLEGSKDALLCGF | hNAv1.7D1 E3 loop |
| | STDSGQCPEGYTCVKIGRNPDYGYTSFDTFSWAFLALFRLMTQDYWENL | |
| | YQQTLRAAGK | |
| 6 | H-CVEIFLADVEG-NH₂ | KP1.1 |
| 7 | MAMEHHPMTEEFK | hNAv1.7D2 E1 loop |
| 8 | VE1FLADVEG | hNAv1.7D2 E2 loop |
| 9 | GKSYKECVCKINDDCTLPRWHMNDFFHSFLIVFRVLCGEWIETMWDCMEVA | hNAv1.7D2 E3 loop |
| | GQA | |
| 10 | H-VEIFLADVEGC-NH₂ | KP1.2 |
| 11 | LAFEDIYIERKKTIK | hNAv1.7D3 E1 loop |
| 12 | VTLVANTLGYSDLGPIK | hNAv1.7D3 E2 loop |
| 13 | AGKFYECINTTIDGSRFPASQVPNRSECFALMNVSQNVRWKNLINNFDNV | hNAv1.7D3 E3 loop |
| | GLGYLSLLQVATFKGWTIIMYAAVDSVNVDKQPKYEYSL | |
| 14 | H-CVEIFLADVEGC-NH₂ | KP1.3 (cyclic) |
| 15 | MMVEKEGQSQHMTE | hNAv1.7D4 E1 loop |
| 16 | VGMFLADLIETYFVSPTLFR | hNAv1.7D4 E2 loop |
| 17 | AYVKKEDGINDMFNFETFGNSMICLFQITTSAGWDGLLAPILNSKPPDCD | hNAv1.7D4 E3 loop |
| | PKINHPGSSVEGDCGNPS | |
| 18 | H-CVGMFLADLIETYFVSPTL-NH₂ | KP2.1 |
| 19 | ATGGAATTCCCCATTGGATCCCTCGAAACTAACAACTTCCGTCGCTTTA | hNAv1.8-nucleotide |
| | CTCCGGAGTCACTGGTGGAGATAGAGAAGCAAATTGCTGCCAAGCAG | NM_006514 |
| | GGAACAAAGAAAGCCAGAGAGAAGCATAGGGAGCAGAAGGACCAAGA | |
| | AGAGAAGCCTCGGCCCCAGCTGGACTTGAAAGCCTGCAACCAGCTGCC | |
| | CAAGTTCTATGGTGAGCTCCCAGCAGAACTGATCGGGGAGCCCCTGGA | |
| | GGATCTAGATCCGTTCTACAGCACACACCGGACATTTATGGTGCTGAA | |
| | CAAAGGGAGGACCATTTCCCGGTTTAGTGCCACTCGGGCCCTGTGGCT | |
| | ATTCAGTCCTTTCAACCTGATCAGAAGAACGGCCATCAAAGTGTCTGT | |
| | CCACTCGTGGTTCAGTTTATTTATTACGGTCACTATTTTGGTTAATTGT | |
| | GTGTGCATGACCCGAACTGACCTTCCAGAGAAAATTGAATATGTCTTC | |
| | ACTGTCATTTACACCTTTGAAGCCTTGATAAAGATACTGGCAAGAGGA | |
| | TTTTGTCTAAATGAGTTCACGTACCTGAGAGATCCTTGGAACTGGCTG | |
| | GATTTTAGCGTCATTACCCTGGCATATGTTGGCACAGCAATAGATCTC | |
| | CGTGGGATCTCAGGCCTGCGGACATTCAGAGTTCTTAGAGCATTAAAA | |
| | ACAGTTCTGTGATCCCAGGCCTGAAGGTCATTGTGGGGGCCCTGATT | |
| | CACTCAGTGAAGAAACTGGCTGATGTGACCATCCTCACCATCTTCTGC | |
| | CTAAGTGTTTTTGCCTGGTGGGGCTGCAACTCTTCAAGGGCAACCTC | |
| | AAAAATAAATGTGTCAAGAATGACATGGCTGTCAATGAGACAACCAAC | |
| | TACTCATCTCACAGAAAACCAGATATCTACATAAATAAGCGAGGCACTT | |
| | CTGACCCCTTACTGTGTGGCAATGGATCTGACTCAGGCCACTGCCCTG | |
| | ATGGTTATATCTGCCTTAAAACTTCTGACAACCCGGATTTTAACTACAC | |
| | CAGCTTTGATTCCTTTGCTTGGGCTTTCCTCTCACTGTTCCGCCTCATG | |
| | ACACAGGATTCCTGGGAACGCCTCTACCAGCAGACCCTGAGGACTTCT | |
| | GGGAAAATCTATATGATCTTTTTTGTGCTCGTAATCTTCCTGGGATCT1 | |
| | TCTACCTGGTCAACTTGATCTTGGCTGTAGTCACCATGGCGTATGAGG | |
| | AGCAGAACCAGGCAACCACTGATGAAATTGAAGCAAAGGAGAAGAAGT | |
| | TCCAGGAGGCCCTCGAGATGCTCCGGAAGGAGCAGGAGGTGCTAGCA | |
| | GCACTAGGGATTGACACAACCTCTCTCCACTCCCACAATGGATCACCTT | |
| | TAACCTCCAAAAATGCCAGTGAGAGAAGGCATAGAATAAAGCCAAGAG | |
| | TGTCAGAGGGCTCCACAGAAGACAACAAATCACCCCGCTCTGATCCTT | |
| | ACAACCAGCGCAGGATGTCTTTTCTAGGCCTCGCCTCTGGAAAACGCC | |
| | GGCTAGTCATGGCAGTGTGTTCCATTTCCGGTCCCCTGGCCGAGATA | |
| | TCTCACTCCCTGAGGGAGTCACAGATGATGGAGTCTTTCCTGGAGACC | |
| | ACGAAAGCCATCGGGGCTCTCTGCTGCTGGGTGGGGGTGCTGGCCAG | |
| | CAAGGCCCCCTCCCTAGAAGCCCTCTCCTCAACCCAGCAACCCTGACT | |
| | CCAGGCATGGAGAAGATGAACACCAACCGCCGCCCACTAGTGAGCTTG | |
| | CCCCTGGAGCTGTCGATGTCTCGGCATTCGATGCAGGACAAAAGAAGA | |
| | CTTTCTTGCAGCAGAATACTTAGATGAACLTTTCCGGGCCCAAAGGG | |
| | CAATGAGTGTTGTCAGTATCATAACCTCCGTCCTTGAGGAACTCGAGG | |
| | AGTCTGAACAGAAGTGCCCACCCTGCTTGACCAGCTTGTCTCAGAAGT | |
| | ATCTGATCTGGGATTGCTGCCCCATGTGGGTGAAGCTCAAGACAATTC | |
| | TCTTGGGCTTGTGACGGATCTTTGCAGAGCTCACCATCACCTTGT | |
| | GCATCGTGGTGAACACCATCTTCATGGCCATGGAGCACCATGGCATGA | |
| | GCCCTACCTTCGAAGCCATGCTCCAGATAGGCAACATCGTCTTTACCA | |
| | TATTTTTTACTGCTGAAATGGTCTCAAAATCATTGCCTCGACCCATA | |
| | CTATTATTTCCAGAAGAAGTGGAATATCTTTGACTGCATCATCGTCACT | |
| | GTGAGTCTGCTAGAGCTGGGCGTGGCCAAGAAGGGAAGCCTGTCTGT | |
| | GCTGCGGAGCTTCCGCTTGCTGCGCGTATTCAAGCTGGCCAAATCCTG | |
| | GCCCACCTTAAACACACTCATCAAGATCATCGGAAACTCAGTGGGGGC | |
| | ACTGGGGAACCTCACCATCATCCTGGCCATCATTGTCTTTGTCTTTGCT | |
| | CTGGTTGGCAAGCAGCTCCTAGGGGAAAACTACCGTAACAACCGAAAA | |
| | AATATCTCCGCGCCCCATGAAGACTGGCCCCGCTGGCACATGCACGAC | |
| | TCTCCACTCTTTCCTCATTGTCTCCGTATCCCTGTGGAGAGTGGA | |
| | TTGAGAACATGTGGGCCTGCATGGAAGTTGGCCAAAAATCCATATGCC | |
| | TCATCCTTTTCTTGACGGTGATGGTGCTAGGGAACCTGGTGGTGCTTA | |
| | ACCTGTTCATCGCCCTGCTATTGAACTCTTTCAGTGCTGACAACCTCAC | |
| | AGCCCCGGAGGACGATGGGGAGGTGAACAACCTGCAGGTGGCCCTGG | |
| | CACGGATCCAGGTCTTTGGCCATCGTACCAAACAGGCTCTTTGCAGCT | |
| | TCTTCAGCAGGTCCTGCCCATTCCCCCAGCCCAAGGCAGAGCCTGAGC | |
| | TGGTGGTGAAACTCCCACTCTCCAGCTCCAAGGCTGAGAACCACATTG | |
| | CTGCCAACACTGCCAGGGGGAGCTCTGGAGGGCTCCAAGCTCCCAGA | |
| | GGCCCCAGGGATGAGCACAGTGACTTCATCGCTAATCCGACTGTGTGG | |
| | GTCTCTGTGCCCATTGCTGAGGGTGAATCTGATCTTGATGACTTGGAG | |
| | GATGATGGTGGGGAAGATGCTCAGAGCTTCCAGCAGGAAGTGATCCC | |
| | CAAAGGACAGCAGGAGCAGCTGCAGCAAGTCGAGAGGTGTGGGGACC | |
| | ACCTGACACCCAGGAGCCCAGGCACTGGAACATCTTCTGAGGACCTGG | |
| | CTCCATCCCTGGGTGAGACGTGGAAAGATGAGTCTGTTCCTCAGGTCC | |
| | CTGCTGAGGGAGTGGACGACACAAGCTCCTCTGAGGGCAGCACGGTG | |
| | GACTGCCTAGATCCTGAGGAAATCCTGAGGAAGATCCCTGAGCTGGCA | |
| | GATGACCTGGAAGAACCAGATGACTGCTTCACAGAAGGATGCATTCGC | |
| | CACTGTCCCTGCTGCAAACTGGATACCACCAAGAGTCCATGGGATGTG | |
| | GGCTGGCAGGTGCGCAAGACTTGCTACCGTATCGTGGAGCACAGCTG | |
| | GTTTGAGAGCTTCATCATCTTCATGATCCTGCTCAGCAGTGGATCTCT | |
| | GGCCTTTGAAGACTATTACCTGGACCAGAAGCCCACGGTGAAAGCTTT | |
| | GCTGGAGTACACTGACAGGGTCTTCACCTTTATCTTTGTGTTCGAGAT | |
| | GCTGCTTAAGTGGGTGGCCTATGGCTTCAAAAAGTACTCACCAATGC | |
| | CTGGTGCTGGCTGGACTTCCTCATTGTGAATATCTCACTGATAAGTCT | |
| | CACAGCGAAGATTCTGGAATATTCTGAAGTGGCTCCCATCAAAGCCCT | |
| | TCGAACCCTCGCGCTCTGCGGCCACTGCGGGCTGGCTCGATTTGA | |
| | AGGCATGCGGGTGGTGGTGGATGCCCTGGTGGGCGCCATCCCATCCA | |
| | TCATGAATGTCCTCCTCGTCTGCCTCATCTTCTGGCTCATCTTCAGCAT | |
| | CATGGGTGTGAACCCTCGCAGGGAAGTTTGGAGGTGCATCAACTA | |
| | TACCGATGGAGAGTTTTCCCTGTACCMGTCGATTGTGAATAACAA | |
| | GTCTGACTGCAAGATTCAAAACTCCACTGGCAGCTCTTCTGGGTCAA | |
| | TGTGAAAGTCAACTTTGATAATGTTGCAATGGGTTACCTGCACTCT | |
| | GCAGGTGGCAACCTTTAAAGGCTGGATGGACATTATGTATGCAGCTGT | |
| | TGATTCCCGGGAGGTCAACATGCAACCCAAGTGGGAGGACAACGTGTA | |
| | CATGTATTGTACTTTGCATCTTCATCATTTTTGGAGGCTTCTTCACA | |
| | CTGAATCTCTTGTGGGGTCATAATTGACAACTTCAATCAACAGAAAA | |
| | AAAAGTTAGGGGGCCAGGACATCTTCATGACAGAGGAGCAGAAGAAAT | |
| | ACTACAATGCCATGAAGAAGTTGGGCTCCAAGAAGCCCCAGAAGCCCA | |
| | TCCCACGGCCCCTGAACAAGTTCCAGGGTTTTGTCTTTGACATCGTGA | |
| | CCAGACAAGCTTTTGACATCACCATCATGGTCCTCATCTGCCTCAACAT | |
| | GATCACCATGATGGTGGAGACTGATGACCAAAGTGAAGAAAAGACGAA | |
| | AATTCTGGGCAAAATCAACCAGTTCTTTGTGGCCGTCTTCACAGGCGA | |
| | ATGTGTCATGAAGATGTTCGCTTTGAGGCAGTACTACTCACAAATGG | |
| | CTGGAATGTGTTTGACTCATTGTGGGGTCTCTCCATTGCGAGCCT | |
| | GATTTTTTCTGCAATTCTTAAGTCACTCAAAGTTACTTCTCCCCAACG | |
| | CTCTCAGAGTCATCCGCCTGGCCCGAATTGGCCGCATCCTCAGACTG | |
| | ATCCGAGCGGCCAAGGGGATCCGCACACTGCTCTTTGCCCTCATGATG | |
| | TCCCTGCCTGCCCTCTTCAACATCGGGCTGTTGCTATTCCTTGTCATGT | |
| | TCATCTACTCTATCTTCGGTATGTCCAGTTTCCCCATGTGAGGTGGG | |
| | AGGCTGGCATCGACGACATGTTCAACTTCCAGACCTTCGCCAACAGCA | |
| | TGCTGTGCCTCTCCAGATTACCACGTCGGCCGGCTGGGATGGCCTCC | |
| | TCAGCCCCATCCTCAACACAGGGCCCCCCTACTGTGACCCCAATCTGC | |
| | CCAACAGCAATGGCACCAGAGGGGACTGTGGGAGCCCAGCCGTAGGC | |
| | ATCATCTCTTCACCACCTACATCATCATCTCCTTCCTCATCATGGTCA | |
| | ACATGTACATTGCAGTGATTCTGGAGAACTTCAATGTGGCCACGGAGG | |
| | AGAGCACTGAGCCCCTGAGTGAGGACGACTTTGACATGTTCTATGAGA | |
| | CCTGGGAGAAGTTTGACCCAGAGGCCACTCAGTTTATTACCTTTTCTG | |
| | CTCTCTCGGACTTTGCAGACACTCTCTCTGGTCCCCTGAGAATCCCAAA | |
| | ACCCAATCGAAATATACTGATCCAGATGGACCTGCCTTTGGTCCCTGG | |
| | AGATAAGATCCACTGCTTGGACATCCTTTTTGCTTTCACCAAGAATGTC | |
| | CTAGGAGAATCCGGGGAGTTGGATTCTCTGAAGGCAAATATGGAGGA | |
| | GAATTTTATGGCAACTAATCTTTCAAAATCATCCTATGAACCAATAGCA | |
| | ACCACTCTCCGATGGAAGCAAGAAGACATTTCAGCCACTGTCATTCAA | |
| | AAGGCCTATCGGAGCTATGTGCTGCACCGCTCCATGGCACTCTCTAAC | |
| | ACCCCATGTGTGCCCAGAGCTGAGGAGGAGGCTGCATCACTCCCAGAT | |
| | GAAGGTTTTGTTGCATTCACAGCAAATGAAAATTGTGTACTCCCAGAC | |
| | AAATCTGAAACTGCTTCTGCCACATCATTCCCACCGTCCTATGAGAGTG | |
| | TCACTAGAGGCCTTAGTGATAGAGTCAACATGAGGACATCTAGCTCAA | |
| | TACAAAATGAAGATGAAGCCACCAGTATGGAGCTGATTGCCCCTGGGC | |
| | CCTAGTGA | |
| 20 | MEFPIGSLETNNFRRFTPESLVEIEKQIAAKQGTKKAREKHREQKDQEEK | hNAv1.8-amino acid |
| | PRPQLDLKACNQLPKFYGEIPAEIIGEPLEDLDPFYSTHRTFMVLNKGRTI | NP_006505 |
| | SRFSATRALWLFSPFNLIRRTAIKVSVHSWFSLFTEVTILVNCVCMTRTDL | |
| | PEKIEYVFTVIYTFEALIKILARGFCLNEFTYLRDPWNWLDFSVITLAYVGT | |
| | AIDLRGISGLRTFRVLRALKTVSVIPGLINIVGALIHSVKKLADVTILTIFCL | |
| | SVFALVGLQLFKGNLKNKCVKNDMAVNETTNYSSHRKPDIYINKRGTSDP | |
| | LLCGNGSDSGHCPDGYICLKTSDNPDFNYTSFDSFAWAFLSLFRLMTQD | |
| | SWERLYQQTLRTSGKIYMIFFVLVIFLGSFYLVNLILAVVTMAYEEQNQAT | |
| | TDEIEAKEKKFQEALEMLRKEQEVLAALGIDTTSLHSHNGSPLTSKNASE | |
| | RRHRIKPRVSEGSTEDNKSPRSDPYNQRRMSFLGLASGKRRASHGSVFH | |
| | FRSPGRDISLPEGVTDDGVFPGDHESHRGSLLLGGGAGQQGPLPRSPLP | |
| | QPSNPDSRHGEDEHQPPPTSEIAPGAVDVSAFDAGQKKTFLSAEYLDEP | |
| | FRAQRAMSVVSIITSVLEEIEESEQKCPPCLTSLSQKYLIWDCCPMVVVKL | |
| | KTILFGLVTDPFAELITTLCIVVNTIFMAMEHHGMSPTFEAMLQIGNIVFTI | |
| | FFTAEMVFKIIAFDPYYYFQKKWNIFDCIIVIVSLLEIGVAKKGSLSVLRSF | |
| | RLLRVFKLAKSWPTLNTLIKIIGNSVGALGNLTIILAIIVFVFALVGKQLLGE | |
| | NYRNNRKNISAPHEDWPRWHMHDFFHSFLIVFRILCGEWIENMWACME | |
| | VGQKSICLILFLTVMVLGNLVVLNLFIALLLNSFSADNLTAPEDDGEVNNL | |
| | QVALARIQVFGHRTKQALCSFFSRSCPFPQPKAEPEIVVKLPLSSSKAENH | |
| | IAANTARGSSGGLQAPRGPRDEHSDFIANPTVWVSVPIAEGESDLDDLE | |
| | DDGGEDAQSFQQEVIPKGQQEQLQQVERCGDHLTPRSPGTGTSSEDLA | |
| | PSLGETWKDESVPQVPAEGVDDISSSEGSTVDCLDPEEILRKIPE1ADDL | |
| | EEPDDCFTEGCIRHCPCCKLDTTKSPWDVGWQVRKTCYRIVEHSWFESF | |
| | IIFMILLSSGSLAFEDYYLDQKPTVKALLEYTDRVFTFIFVFEMLLKVVVAYG | |
| | FKKYFTNAWCWLDFLIVNISLISLTAKILEYSEVAPIALRTLRALRPLRAL | |
| | SRFEGMRVVVDALVGAIPSIMNVLLVCLIFWLIFSIMGVNLFAGKFWRCIN | |
| | YTDGEFSLVPLSIVNNKSDCKIQNSTGSFFWVNVKVNFDNVAMGYLALL | |
| | QVATFKGWMDIMYAAVDSREVNMQPKWEDNVYMYLYFVIFIIFGGFFTL | |
| | NLFVGVIIDNFNQQKKKLGGQDIFMTEEQKKYYNAMKKLGSKKPQKPIPR | |
| | PLNKFQGFVFDIVTRQAFDITIMVLICLNMITMMVETDDQSEEKTKILGKI | |
| | NQFFVAVFTGECVMKMFALRQYYFTNGWNVFDFMNLSIASLIFSAILKS | |
| | LQSYFSPTLFRVIRLARIGRILRLIRAAKGIRTLLFALMMSLPALFNIGLLLF | |
| | LVMFIYSIFGMSSFPHVRWEAGIDDMFNFQTFANSMLCLFQITTSAGWD | |
| | GLLSPILNTGPPYCDPNLPNSNGTRGDCGSPAVGIIFFTTYIIISFLIMVNM | |
| | YIAVILENFNVATEESTEPLSEDDFDMFYETVVEKFDPEATQFITFSALSDF | |
| | ADTLSGPLRIPKPNRNILIQMDLPLVPGDKIHCLDILFAFTKNVLGESGE1 | |
| | DSLKANMEEKFMATNLSKSSYEPIATTLRWKQEDISATVIQKAYRSYVLH | |
| | RSMALSNTPCVPRAEEEAASLPDEGFVAFTANENCVLPDKSETASATSFP | |
| | PSYESVTRGLSDRVNMRTSSSIQNEDEATSMEIIAPGP | |
| 21 | CMTRTDLP | hNAv1.8D1 E1 loop |
| 22 | VGTAIDLRG | hNAv1.8D1 E2 loop |
| 23 | KGNLKNKCVKNDMAVNETTNYSSHRKPDIYINKRGTSDPLLCGNGSDSG | hNAv1.8D1 E3 loop |
| | HCPDGYICLKTSDNPDFNYTSFDSFAWAFLSLFRLMTQDSWERLYQQTL | |
| | RTSGK | |
| 24 | H-VGMFLADLIETYFVSPTLC-NH₂ | KP2.2 |
| 25 | MAMEHHGMSPTFE | hNAv1.8D2 E1 loop |
| 26 | LEIGVAKKGS | hNAv1.8D2 E2 loop |
| 27 | GENYRNNRKNISAPHEDWPRWHMHDFFHSFLIVFRILCGEWIENMWAC | hNAv1.8D2 E3 loop |
| | MEVGQKS | |
| 28 | H-CVGMFLADLIETYFVSPTLC-NH₂ | KP2.3 (cyclic) |
| 29 | LAFEDYYLDQKPTVK | hNAv1.8D3 E1 loop |
| 30 | ISLTAKILEYSEVAPIK | hNAv1.8D3 E2 loop |
| 31 | AGKFWRCINYTDGEFSLVPLSIVNNKSDCKIQNSTGSFFWVNVKVNFDN | hNAv1.8D3 E3 loop |
| | VAMGYLALLQVATFKGWMDIMYAAVDSREVNMQPKWEDNV | |
| 32 | H-CLTEFVNLGN-NH₂ | KP5.1 |
| 33 | MMVETDDQSEEKTK | hNAv1.8D4 E1 loop |
| 34 | ASLIFSAILKSLQSYFSPTLFR | hNAv1.8D4 E2 loop |
| 35 | PHVRWEAGIDDMFNFQTFANSMLCLFQITTSAGWDGLLSPILNTGPPYC | hNAv1.8D4 E3 loop |
| | DPNLPNSNGTRGDCGSPA | |
| 36 | H-LTEFVNLGNC-NH₂ | KP5.2 |
| 37 | ATGGATGACAGATGCTACCCAGTAATCTTTCCAGATGAGCGGAATTTC | hNAv1.9-nucleotide |
| | CGCCCCTTCACTTCCGACTCTCTGGCTGCAATTGAGAAGCGGATTGCC | NM_014139 |
| | ATCCAAAAGGAGAAAAAGAAGTCTAAAGACCAGACAGGAGAAGTACCC | |
| | CAGCCTCGGCCTCAGCTTGACCTAAAGGCCTCCAGGAAGTTGCCCAAG | |
| | CTCTATGGCGACATTCCTCGTGAGCTCATAGGAAAGCCTCTGGAAGAC | |
| | TTGGACCCATTCTACCGAAATCATAAGACATTTATGGTGTTAAACAGAA | |
| | AGAGGACAATCTACCGCTTCAGTGCCAAGCATGCCTTGTTCATTTTG | |
| | GGCCTTTCAATTCAATCAGAAGTTTAGCCATTAGAGTCTCAGTCCATTC | |
| | ATTGTTCAGCATGTTCATTATCGGCACCGTTATCATCAACTGCGTGTTC | |
| | ATGGCTACAGGGCCTGCTAAAAACAGCAACAGTAACAATACTGACATT | |
| | GCAGAGTGTGTCTTCACTGGGATTTATATTTTTGAAGCTTTGATTAAA | |
| | ATATTGGCAAGAGTTTTTCATTCTGGATGAGTTTTCTTTCCTTCGAGAT | |
| | CCATGGAACTGGCTGGACTCCATTGTCATTGGAATAGCGATTGTGTCA | |
| | TATATTCCAGGAATCACCATCAAACTATTGCCCCTGCGTACCTTCCGTG | |
| | TGTTCAGAGCTTTGAAAGCAATTTCAGTAGTTTCACGTCTGAAGGTCA | |
| | TCGTGGGGGCCTTGCTACGCTCTGTGAAGAAGCTGGTCAACGTGATTA | |
| | TCCTCACCTTCTTTTGCCTCAGCATCTTTGCCCTGGTAGGTCAGCAGCT | |
| | CTTCATGGGAAGTCTGAACCTGAAATGCATCTCGAGGGACTGTAAAAA | |
| | TATCAGTAACCCGGAAGCTTATGACCATTGCTTTGAAAAGAAAGAAAA | |
| | TTCACCTGAATTCAAAATGTGTGGCATCTGGATGGGTAACAGTGCCTG | |
| | TTCCATACAATATGAATGTAAGCACACCAAAATTAATCCTGACTATAAT | |
| | TATACGAATTTTGACAACTTTGGCTGGTCTTTTCTTGCCATGTTCCGG | |
| | CTGATGACCCAAGATTCCTGGGAGAAGCTTTATCAACAGACCCTGCGT | |
| | ACTACTGGGCTCTACTCAGTCTTCTTCTTCATTGTGGTCATTTTCCTGG | |
| | GCTCCTTCTACCTGATTAACTTAACCCTGGCTGTTGTTACCATGGCATA | |
| | TGAGGAGCAGAACAAGAATGTAGCTGCAGAGATAGAGGCCAAGGAAA | |
| | AGATCGTTTCAGGAAGCCCAGCAGCTGTTAAAGGAGGAAAAGGAGGCT | |
| | CTGGTTGCCATGGGAATTGACAGAAGTTCACTTACTTCCCTTGAAACA | |
| | TCATTTTACCCCAAAAAAGAGAAAGCTCTTTGGTAATAAGAAAAGGA | |
| | AGTCCTTCTTTTTGAGAGAGTCTGGGAAAGACCAGCCTCCTGGGTCAG | |
| | ATTCTGATGAAGATTGCCAAAAAAAGCCACAGCTCCTAGAGCAAACCA | |
| | AACGACTGTCCCAGAATCTATCACTGGACCACTTTGATGAGCATGGAG | |
| | ATCCTCTCCAAAGGCAGAGAGCACTGAGTGCTGTCAGCATCCTCACCA | |
| | TCACCATGAAGGAACAAGAAAAATCACAAGAGCCTTGTCTCCCTTGTG | |
| | GAGAAAACCTGGCATCCAAGTACCTCGTGTGGAACTGTTGCCCCCAGT | |
| | GGCTGTGCGTTAAGAAGGTCCTGAGAACTGTGATGACTGACCCGTTTA | |
| | CTGAGCTGGCCATCACCATCTGCATCATCATCAACACTGTCTTCTTGGC | |
| | CATGGAGCATCACAAGATGGAGGCCAGTTTTGAGAAGATGTTGAATAT | |
| | AGGGAATTTGGTTTTCACTAGCATTTTTATAGCAGAAATGTGCCTAAA | |
| | AATCATTGCGCTCGATCCCTACCACTACTTTCGCCGAGGCTGGAACAT | |
| | TTTTGACAGCATTGTTGCTCTTCTGAGTTTTGCAGATGTAATGAACTG | |
| | TGTACTTCAAAAGAGAAGCTGGCCATTCTTGCGTTCAAACAGAGTGCT | |
| | CAGGGTCTTCAAGTTAGCCAAATCCTGGCCAACTTTGAACACACTAKTT | |
| | AAGATAATCGGCAACTCTGTCGGAGCCCTTGGAAGCCTGACTGTGGTC | |
| | CTGGTCATTGTGATCTTTATTTTCTCAGTAGTTGGCATGCAGCTTTTT | |
| | GGCCGTAGCTTCAATTCCCAAAAGAGTCCAAAACTCTGTAACCCGACA | |
| | GGCCCGACAGTCTCATGTTTACGGCACTGGCACATGGGGGATTTCTGG | |
| | CACTCCTTCCTAGTGGTATTCCGCATCCTCTGCGGGGAATGGATCGAA | |
| | AATATGTGGGAATGTATGCAAGAAGCGAATGCATCATCATCATTGTGT | |
| | GTTATTGTCTTCATATTGATCACGGTGATAGGAAAACTTGTGGTGCTC | |
| | ACCTCTTCATTGCCTTACTGCTCAATTCCTTTAGCAATGAGGAAAGAA | |
| | ATGGAAACTTAGAAGGAGAGGCCAGGAAAACTAAAGTCCAGTTAGCAC | |
| | GGATCGATTCCGCCGGGCTTTTTGTTTTGTGAGACACACTCTTGAGC | |
| | ATTTCTGTCACAAGTGGTGCAGGAAGCAAAACTTACCACAGCAAAAAG | |
| | AGGTGGCAGGAGGCTGTGCTGCACAAAGCAAAGACATCATTCCCCTGG | |
| | TCATGGAGATGAAAAGGGGCTCAGAGACCCAGGAGGAGCTTGGTATA | |
| | CTAACCTCTGTACCAAAGACCCTGGGCGTCAGGCATGATTGGACTTGG | |
| | TTGGCACCACTTGCGGAGGAGGAAGATGACGTTGAATTTTCTGGTGAA | |
| | GATAATGCACAGCGCATCACACAACCTGAGCCTGAACAACAGGCCTAT | |
| | GAGCTCCATCAGGAGAACAAGAAGCCCACGAGCCAGAGAGTTCAAAGT | |
| | GTGGAAATTGACATGTTCTCTGAAGATGAGCCTCATCTGACCATACAG | |
| | GATCCCCGAAAGAAGTCTGATGTTACCAGTATACTATCAGAATGTAGC | |
| | ACCATTGATCTTCAGGATGGCTTTGGATGGTTACCTGAGATGGTTCCC | |
| | AAAAAGCAACCAGAGAGATGTTTGCCCAAAGGCTTTGGTTGCTGCTTT | |
| | CCATGCTGTAGCGTGGACAAGAGAAAGCCTCCCTGGGTCATTTGGTGG | |
| | AACCTGCGGAAAACCTGCTACCAAATAGTGAAACACAGCTGGTTTGAG | |
| | AGCTTTATTATCTTTGTGATTCTGCTGAGCAGTGGGGCACTGATATTT | |
| | GAAGATGTTCACCTTGAGAACCAACCCAAAATCCAAGAATTACTAAATT | |
| | GTACTGACATTATTTTTACACATATTTTTATCCTGGAGATGGTACTAAA | |
| | ATGGGTAGCCTTCGGATTTGGAAAGTATTTCACCAGTGCCTGGTGCTG | |
| | CCTTGATTTCATCATTGTGATTGTCTCTGTGACCACCCTCATTAACTTA | |
| | ATGGAATTGAAGTCCTTCCGGACTCTACGAGCACTGAGGCCTCTTCGT | |
| | GCGCTGTCCCAGTTTGAAGGAATGAAGGTGGTGGTCAATGCTCTCATA | |
| | GGTGCCATACCTGCCATTCTGAATGTTTTGCTTGTCTGCCTCATTTTCT | |
| | GGCTCGTATTTTGTATTCTGGGAGTATACTTCTTTTCTGGAAAATTTG | |
| | GGAAATGCATTAATGGAACAGACTCAGTTATAAATTATACCATCATTAC | |
| | AAATAAAAGTCAATGTGAAAGTGGCAATTTCTCTTGGATCAACCAGAA | |
| | AGTCAACTTTGACAATGTGGGAAATGCTTACCTCGCTCTGCTGCAAGT | |
| | GGCAACATTTAAGGGCTGGATGGATATTATATATGCAGCTGTTGATTC | |
| | CACAGAGAAAGAACAACAGCCAGAGTTTGAGAGCAATTCACTCGGTTA | |
| | CATTTACTTCGTAGThTTTATCATCTTTGGCTCATTCTTCACTCTGAAT | |
| | CTCTTCATTGGCGTTATCATTGACAACTTCAACCAACAGCAGAAAAAGT | |
| | TAGGTGGCCAAGACATTTTTATGACAGAAGAACAGAAGAAATACTATA | |
| | ATGCAATGAAAAAATTAGGATCCAAAAAACCTCAAAAACCCATTCCACG | |
| | GCCTCTGAACAAATGTCAAGGTCTCGTGTTCGACATAGTCACAAGCCA | |
| | GATCTTTGACATCATCATCATAAGTCTCATTATCCTAAACATGATTAGC | |
| | ATGATGGCTGAATCATACAACCAACCCAAAGCCATGAAATCCATCCTTG | |
| | ACCATCTCAACTGGGThTTTGTGGTCATCTTTACGTTAGAATGTCTCAT | |
| | CAAAATCTTTGTTTGAGGCAATACTACTTCACCAATGGCTGGAATTTA | |
| | TTTGACTGTGTGGTCGTGCTTCTTTCCATTGTTAGTACAATGATTTCTA | |
| | CCTTGGAAAATCAGGAGCACATTCCTTTCCCTCCGACGCTCTTCAGAAT | |
| | TGTCCGCTTGGCTCGGATTGGCCGAATCCTGAGGCTTGTCCGGGCTGC | |
| | ACGAGGAATCAGGACTCTCCTCTTTGCTCTGATGATGTCGCTTCCTTC | |
| | TCTGTTCAACATTGGTCTTCTACTCTTTCTGATTATGTTTATCTATGCC | |
| | ATTCTGGGTATGAACTGGTTTTCCAAAGTGAATCCAGAGTCTGGAATC | |
| | GATGACATATTCAACTTCAAGACTTTTGCCAGCAGCATGCTCTGTCTCT | |
| | TCCAGATAAGCACATCAGCAGGTTGGGATTCCCTGCTCAGCCCCATGC | |
| | TGCGATCAAAAGAATCATGTAACTCTTCCTCAGAAAACTGCCACCTCCC | |
| | TGGCATAGCCACATCCTACTTTGTCAGTTACATTATCATCTCCTTTCTC | |
| | ATTGTTGTCAACATGTACATTGCTGTGATTTTAGAGAACTTCAATACAG | |
| | CCACTGAAGAAAGTGAGGACCCTTTGGGTGAAGATGALTTTGACATAT | |
| | TTTATGAAGTGTGGGAAAAGTTTGACCCAGAAGCAACACAATTTATCA | |
| | AATATTCTGCCCTTTCTGATTTGCTGATGCCTTGCCTGAGATTTGC | |
| | GTGTCGCAAAGCCAAATAAATATCAATTTCTAGTAATGGACTTGCCCAT | |
| | GGTGAGTGAAGATCGCCTCCACTGCATGGATATTCTTTTCGCCTTCAC | |
| | CGCTAGGGTACTCGGTGGCTCTGATGGCCTAGATAGTATGAAAGCAAT | |
| | GATGGAAGAGAAGTTCATGGAAGCCAATCCTCTCAAGAAGTTGTATGA | |
| | ACCCATAGTCACCACCACCAAGAGAAAGGAAGAGGAAAGAGGTGCTGC | |
| | TATTATTCAAAAGGCCTTTCGAAAGTACATGATGAAGGTGACCAAGGG | |
| | TGACCAAGGTGACCAAAATGACTTGGAAAACGGGCCTCATTCACCACT | |
| | CCAGACTTTTTGCAATGGAGACTTGTCTAGCTTTGGGGTGGCCAAGGG | |
| | CAAGGTCCACTGTGACTGA | |
| 38 | MDDRCYPVIFPDERNFRPFTSDSLAAIEKRIAIQKEKKKSKDQTGEVPQP | hNAv1.9-amino acid |
| | RPQLDLKASRKLPKLYGDIPRE1IGKPLEDLDPFYRNHKTFMVLNRKRTIY | NP_054858 |
| | RFSAKHALFIFGPFNSIRSLAIRVSVHSLFSMFIIGTVIINCVFMATGPAKN | |
| | SNSNNTDIAECVFTGIYIFEALIKILARGFILDEFSFLRDPWNWLDSIVIGI | |
| | AIVSYIPGITIKLLPLRTFRVFRALMISVVSRLKVIVGALLRSVKKLVNVIIL | |
| | TFFCLSIFALVGQQLFMGSLNLKCISRDCKNISNPEAYDHCFEKKENSPEF | |
| | KMCGIWMGNSACSIQYECKHTKINPDYNYTNFDNFGWSFLAMFRLMTQ | |
| | DSWEKLYQQTLRTTGLYSVFFFIVVIFLGSFYLINLTLAVVTMAYEEQNKN | |
| | VAAEIEAKEKMFQEAQQLLKEEKEALVAMGIDRSSLTSLETSYFTPKKRKL | |
| | FGNKKRKSFFLRESGKDQPPGSDSDEDCQKKPQLLEQTKRLSQNLSLDH | |
| | FDEHGDPLQRQRALSAVSILTITMKEQEKSQEPCLPCGENLASKYLVVVNC | |
| | CPQWLCVKINLRTVMTDPFTE1AMCIIINTVFLAMEHHKMEASFEKML | |
| | NIGNLVFTSIFIAEMCLKIIALDPYHYFRRGWNIFDSIVALLSFADVMNCVL | |
| | QKRSWPFLRSFRVLRVFKLAKSWPTLNTLIKIIGNSVGALGSLTVVLVIVIF | |
| | IFSVVGMQLFGRSFNSQKSPKLCNPTGPTVSCLRHWHMGDFWHSFLVVF | |
| | RILCGEWIENMWECMQEANASSSLCVIVFILITVIGKLVVLNLFIALLLNSF | |
| | SNEERNGNLEGEARKTKVQLALDRFRRAFCFVRHTLEHFCHKWCRKQNL | |
| | PQQKEVAGGCAAQSKDIIPLVMEMKRGSETQEE1GILTSVPKTLGVRHD | |
| | VVTWLAPLAEEEDDVEFSGEDNAQRITQPEPEQQAYE1HQENKKPTSQR | |
| | VQSVEIDMFSEDEPHLTIQDPRKKSDVTSILSECSTIDLQDGFGWLPEMV | |
| | PKKQPERCLPKGFGCCFPCCSVDKRKPPWVIWWNLRKTCYQIVKHSWF | |
| | ESFIIFVILLSSGALIFEDVHLENQPKIQE1LNCTDIIFTHIFILEMVLKWVA | |
| | FGFGKYFTSAWCCLDFIIVIVSVTTLINLME1KSFRTLRALRPLRALSQFEG | |
| | MKVVVNALIGAIPAILNVLLVCLIFWLVFCILGVYFFSGKFGKCINGTDSVI | |
| | NYTIITNKSQCESGNFSWINQKVNFDNVGNAYLALLQVATFKGWMDIIY | |
| | AAVDSTEKEQQPEFESNSLGYIYFVVFIIFGSFFTLNLFIGVIIDNFNQQQK | |
| | KLGGQDIFMTEEQKKYYNAMKKLGSKKPQKPIPRPLNKCQGLVFDIVTSQ | |
| | IFDIIIISLIILNMISMMAESYNQPKAMKSILDHLNWVFWIFTLECLIKIFA | |
| | LRQYYFTNGWNLFDCVVVLLSIVSTMISTLENQEHIPFPPTLFRIVRLARI | |
| | GRILRLVRAARGIRTLLFALMMSLPSLFNIGE1FLIMFIYAILGMNWFSKV | |
| | NPESGIDDIFNFKTFASSMLCLFQISTSAGWDSLLSPMLRSKESCNSSSEN | |
| | CHLPGIATSYFVSYIIISFLIVVNMYIAVILENFNTATEESEDPLGEDDFDIF | |
| | YEVWEKFDPEATQFIKYSALSDFADALPEPLRVAKPNKYQFLVMDLPMVS | |
| | EDRLHCMDILFAFTARVLGGSDGLDSMKAMMEEKFMEANPLKKLYEPIV | |
| | TTTKRKEEERGAAIIQKAFRKYMMIWTKGDQGDQNDLENGPHSPLQTLC | |
| | NGDLSSFGVAKGKVHCD | |
| 39 | FMATGPAK | hNAv1.9-D1 E1 loop |
| 40 | VSYIPGITIK | hNAv1.9-DI E2 loop |
| 41 | MGSLNLKCISRDCKNISNPEAYDHCFEKKENSPEFKMCGIWMGNSACSI | |
| | QYECKHTKINPDYNYTNFDNFGWSFLAMFRLMTQDSWEKLYQQTLRTT | hNAv1.9-D1 E3 loop |
| | GL | |
| 42 | H-CLTEFVNLGNC-NH₂ | KP5.3 (cyclic) |
| 43 | LAMEHHKMEASFE | hNAv1.9-D2 E1 loop |
| 44 | ADVMNCVLQKRS | hNAv1.9-D2 E2 loop |
| 45 | MGSLNLKCISRDCKNISNPEAYDHCFEKKENSPEFKMCGIWMGNSACSIQYE | hNAv1.9-D2 E3 loop |
| | CKHTKINPDYNYTNFDNFGWSFLAMFRLMTQDSWEKLYQQTLRTTGL | |
| 46 | H-CVTLVANTLGYSDLGPI-NH₂ | KP6.1 |
| 47 | LIFEDVHLENQPKIQ | hNAv1.9-D3 E1 loop |
| 48 | TTLINMEIK | hNAv1.9-D3 E2 loop |
| 49 | SGKFGKCINGTDSVINYTIITNKSQCESGNFSWINQKVNFDNVGNAYLALLQ | hNAv1.9-D3 E3 loop |
| | VATFKGWMDIIYAAVDSTEKEQQPEFESNS | |
| 50 | H-VTLVANTLGYSDLGPIC-NH₂ | KP6.2 |
| 51 | MMAESYNQPKAMKS | hNAv1.9-D4 E1 loop |
| 52 | VSTMISTLENQEHIPFPPTLFR | hNAv1.9-D4 E2 loop |
| 53 | SKVNPESGIDDIFNFKTFASSMLCLFQISTSAGWDSLLSPMLRSKESCNSS | hNAv1.9-D4 E3 loop |
| | SENCHLPG | |
| 54 | H-CVTLVANTLGYSDLGPIC-NH₂ | KP6.3 (cyclic) |
| 55 | ATGGAGCAAACAGTGCTTGTACCACCAGGACCTGACAGCTTCAACTTC | hNAv1.1nucleotide |
| | TTCACCAGAGAATCTCTTGCGGCTATTGAAAGACGCATTGCAGAAGAA | |
| | AAGGCAAAGAATCCCAAACCAGACAAAAAAGATGACGACGAAAATGGC | |
| | CCAAAGCCAAATAGTGACTTGGAAGCTGGAAAGAACCTTCCATTTATT | |
| | TATGGAGACATTCCTCCAGAGATGGTGTCAGAGCCCCTGGAGGACCTG | |
| | GACCCCTACTATATCAATAAGAAAATTTTTATAGTATTGAATAAAGGGA | |
| | AGGCCATCTTCCGGTTCAGTGCCACCTCTGCCCTGTACATTTTAACTCC | |
| | CTTCAATCCTCTTAGGAAAATAGCTATTAAGATTTTGGTACATTCATTA | |
| | TTCAGCATGCTAATTATGTGCACTATTTGACAAACTGTGTGTTTATGA | |
| | CAATGAGTAACCCTCCTGATTGGACAAAGAATGTAGAATACACCTTCA | |
| | CAGGAATATATACTTTTGAATCACTTATAAAAATTATTGCAAGGGGATT | |
| | CTGTTTAGMGATTTTAdTTTCCTTCGGGATCCATGGAACTGGCTCGA | |
| | TTTCACTGTCATTACATTTGCGTACGTCACAGAGTTTGTGGACCTGGG | |
| | CAATGTCTCGGCATTGAGAACATTCAGAGTTCTCCGAGCATTGAAGAC | |
| | GATTTCAGTCATTCCAGGCCTGAAAACCATTGTGGGAGCCCTGATCCA | |
| | GTCTGTGAAGAAGCTCTCAGATGTAATGATCCTGACTGTGTTCTGTCT | |
| | GAGCGTATTTGCTCTAATTGGGCTGCAGCTGTTCATGGGCAACCTGAG | |
| | GAATAAATGTATACAATGGCCTCCCACCAATGCTTCCTTGGAGGAACA | |
| | TAGTATAGAAAAGAATATAACTGTGAATTATAATGGTACACTTATAAAT | |
| | GAAACTGTCTTTGAGTTTGACTGGAAGTCATATATTCAAGATTCAAGA | |
| | TATCATTATTTCCTGGAGGGTTTTTTAGATGCACTACTATGTGGAAAT | |
| | AGCTCTGATGCAGGCCAATGTCCAGAGGGATATATGTGTGTGAAAGCT | |
| | GGTAGAAATCCCAATTATGGCTACACAAGCTTTGATACCTTCAGTTGG | |
| | GCTTTTTTGTCCTTGTTTCGACTAATGACTCAGGACTTCTGGGAAAAT | |
| | CTTTATCAACTGACATTACGTGCTGCTGGGAAAACGTACATGATA | |
| | TTGTATTGGTCATTTTCTTGGGCTCATTCTACCTAATAAATTTGATCCT | |
| | GGCTGTGGTGGCCATGGCCTACGAGGAACAGAATCAGGCCACCTTGG | |
| | AAGAAGCAGAACAGAAAGAGGCCGAATTTCAGCAGATGATTGAACAGC | |
| | TTAAAAAGCAACAGGAGGCAGCTCAGCAGGCAGCAACGGCAACTGCCT | |
| | CAGAACATTCCAGAGAGCCCAGTGCAGCAGGCAGGCTCTCAGACAGCT | |
| | CATCTGAAGCCTCTAAGTTGAGTTCCAAGAGTGCTAAGGAAAGAAGAA | |
| | ATCGGAGGAAGAAAAGAAAACAGAAAGAGCAGTCTGGTGGGGAAGAG | |
| | AAAGATGAGGATGAATTCCAAAAATCTGAATCTGAGGACAGCATCAGG | |
| | AGGAAAGGTTTTCGCTTCTCCATTGAAGGGAACCGATTGACATATGAA | |
| | AAGAGGTACTCCTCCCCACACCAGTCTTTGTTGAGCATCCGTGGCTCC | |
| | CTATTTTCACCAAGGCGAAATAGCAGMCAAGCTTTTTCAGCTTTAGA | |
| | GGGCGAGCAAAGGATGTGGGATCTGAGAACGACTTCGCAGATGATGA | |
| | GCACAGCACCTTTGAGGATAACGAGAGCCGTAGAGATTCCTTGTTTGT | |
| | GCCCCGACGACACGGAGAGAGACGCAACAGCAACCTGAGTCAGACCAG | |
| | TAGGTCATCCCGGATGCTGGCAGTGTTTCCAGCGAATGGGAAGATGCA | |
| | CAGCACTGTGGATTGCAATGGTGTGGTTTCCTTGGTTGGTGGACCTTC | |
| | AGTTCCTACATCGCCTGTTGGACAGCTTCTGCCAGAGGTGATAATAGA | |
| | TAAGCCAGCTACTGATGACAATGGAACAACCACTGAAACTGAAATGAG | |
| | AAAGAGAAGGTCAAGTTCTTTCCACGTTTCCATGGACTTTCTAGAAGA | |
| | TCCTTCCCAAAGGCAACGAGCAATGAGTATAGCCAGCATTCTAACAAA | |
| | TACAGTAGAAGAACTTGAAGAATCCAGGCAGAAATGCCCACCCTGTTG | |
| | GTATAAATTTTCCAACATATTCTTAATCTGGGACTGTTCTCCATATTGG | |
| | TTAAAAGTGAAACATGTTGTCAACCTGGTTGTGATGGACCCATTTGTT | |
| | GACCTGGCCATCACCATCTGTATTGTCTTAAATACTCTTTTCATGGCCA | |
| | TGGAGCACTATCCAATGACGGACCATTTCAATAATGTGCTTACAGTAG | |
| | GAAACTTGGTTTTCACTGGGATCTTTACAGCAGAAATGTTTCTGAAAA | |
| | TTATTGCCATGGATCCTTACTATTATTTCCAAGAAGGCTGGAATATCTT | |
| | TGACGGTTTTATTGTGACGCTTAGCCTGGTAGAACTTGGACTCGCCAA | |
| | TGTGGAAGGATTATCTGTTCTCCGTTCATTTCGATTGCTGCGAGTTTT | |
| | CAAGTTGGCAAAATCTTGGCCAACGTTAAATATGCTAATAAAGATCATC | |
| | GGCAATTCCGTGGGGGCTCTGGGAAATTTAACCCTCGTCTTGGCCATC | |
| | ATCGTCTTCATTTTTGCCGTGGTCGGCATGCAGCTCTTTGGTAAAAGC | |
| | TACAAAGATTGTGTCTGCAAGATCGCCAGTGATTGTCAACTCCCACGC | |
| | TGGCACATGAATGACTTCTTCCACTCCTTCCTGATTGTGTTCCGCGTG | |
| | CTGTGTGGGGAGTGGATAGAGACCATGTGGGACTGTATGGAGGTTGC | |
| | TGGTCAAGCCATGTGCCTTACTGTCTTCATGATGGTCATGGTGATTGG | |
| | AAACCTAGTGGTCCTGAATCTCTTTCTGGCCTTGCTTCTGAGCTCATTT | |
| | AGTGCAGACAACCTTGCAGCCACTGATGATGATAATGAAATGAATAAT | |
| | CTCCAAATTGCTGTGGATAGGATGCACAAAGGAGTAGCTTATGTGAAA | |
| | AGAAAAATATATGAATTTATTCAACAGTCCTTCATTAGGAAACAAAAGA | |
| | TTTTAGATGAAATTAAACCACTTGATGATCTAAACAACAAGAAAGACAG | |
| | TTGTATGTCCAATCATACAGCAGAAATTGGGAAAGATCTTGACTATCT | |
| | TAAAGATGTAAATGGAACTACAAGTGGTATAGGAACTGGCAGCAGTGT | |
| | TGAAAAATACATTATTGATGAAAGTGATTACATGTCATTCATAAACAAC | |
| | CCCAGTCTTACTGTGACTGTACCAATTGCTGTAGGAGAATCTGACTTT | |
| | GAAAATTTAAACACGGAAGACTTTAGTAGTGAATCGGATCTGGAAGAA | |
| | AGCAAAGAGAAACTGAATGAAAGCAGTAGCTCATCAGAAGGTAGCACT | |
| | GTGGACATCGGCGCACCTGTAGAAGAACAGCCCGTAGTGGAACCTGAA | |
| | GAAACTCTTGAACCAGAAGCTTGTTTCACTGAAGGCTGTGTACAAAGA | |
| | TTCAAGTGTTGTCAAATCAATGTGGAAGAAGGCAGAGGAAAACAATGG | |
| | TGGAACCTGAGAAGGACGTGTTTCCGAATAGTTGAACATAACTGGTTT | |
| | GAGACCTTCATTGTTTTCATGATTCTCCTTAGTAGTGGTGCTCTGGCA | |
| | TTTGAAGATATATATATTGATCAGCGAAAGACGATTAAGACGATGTTG | |
| | GAATATGCTGACAAGGTTTTCACTTACATTTTCATTCTGGAAATGCTTC | |
| | TAAAATGGGTGGCATATGGCTATCAAACATATTTCACCAATGCCTGGT | |
| | GTTGGCTGGACTTCTTAATTGTTGATGTTTCATTGGTCAGTTTAACAG | |
| | CAAATGCCTTGGGTTACTCAGAACTTGGAGCCATCAAATCTCTCAGGA | |
| | CACTAAGAGCTCTGAGACCTCTAAGAGCCTTATCTCGATTTGAAGGGA | |
| | TGAGGGTGGTTGTGAATGCATTTTAGGAGCAATTCCATCCATCATGA | |
| | ATGTGCTTCTGGTTTGTCTTATATTCTGGCTAATTTTCAGCATCATGG | |
| | GCGTAAATTTGTTTGCTGGCAAATTCTACCACTGTATTAACACCACAAC | |
| | TGGTGACAGGTTTGACATCGAAGACGTGAATAATCATACTGATTGCCT | |
| | AAAACTAATAGAAAGAAATGAGACTGCTCGATGGAAAAATGTGAAAGT | |
| | AAATTTTGATAATGTAGGATTTGGGTATCTCTTTTTGCTTCAAGTTGC | |
| | CACATTCAAAGGATGGATGGATATAATGTATGCAGCAGTTGATTCCAG | |
| | AAATGTGGAACTCCAGCCTAAGTATGAAGAAAGTCTGTACATGTATCT | |
| | TTACTTTGTTATTTTCATCATCTTTGGGTCCTTCTTCACCTTGAACCTG | |
| | TTTATTGGTGTCATCATAGATAATTTCAACCAGCAGAAAAAGAAGTTTG | |
| | GAGGTCAAGACATCTTTATGACAGAAGAACAGAAGAAATACTATAATG | |
| | CAATGAAAAAATTAGGATCGAAAAAACCGCAAAAGCCTATACCTCGAC | |
| | CAGGAAACAAATTTCAAGGAATGGTCTTTGACTTCGTAACCAGACAAG | |
| | TTTTTGACATAAGCATCATGATTCTCATCTGTCTTAACATGGTCACAAT | |
| | GATGGTGGAAACAGATGACCAGAGTGAATATGTGACTACCATTTTGTC | |
| | ACGCATCAATCTGGTGTTCATTGTGCTATTTACTGGAGAGTGTGTACT | |
| | GAAACTCATCTCTCTACGCCATTATTATTTTACCATTGGATGGAATATT | |
| | TTTGATTTTGTGGTTGTCATTCTCTCCATTGTAGGTATGTTTCTTGCCG | |
| | AGCTGATAGAAAAGTATTTCGTGTCCCCTACCCTGTTCCGAGTGATCC | |
| | GTCTTGCTAGGATTGGCCGAATCCTACGTCTGATCAAAGGAGCAAAGG | |
| | GGATCCGCACGCTGCTCTTTGCTTTGATGATGTCCCTTCCTGCGTTGT | |
| | TTAACATCGGCCTCCTACTCTTCCTAGTCATGTTCATCTACGCCATCTT | |
| | TGGGATGTCCAACTTTGCCTATGTTAAGAGGGAAGTTGGGATCGATGA | |
| | CATGTTCAACTTTGAGACCTTTGGCAACAGCATGATCTGCCTATTCCAA | |
| | ATTACAACCTCTGCTGGCTGGGATGGATTGCTAGCACCCATTCTCAAC | |
| | AGTAAGCCACCCGACTGTGACCCTAATAAAGTTAACCCTGGAAGCTCA | |
| | GTTAAGGGAGACTGTGGGAACCCATCTGTTGGAATTTTTTTTTTTGTC | |
| | AGTTACATCATCATATCCTTCCTGGTTGTGGTGAACATGTACATCGCG | |
| | GTCATCCTGGAGAACTTCAGTGTTGCTACTGAAGAAAGTGCAGAGCCT | |
| | CTGAGTGAGGATGATTTGAGATGTTCTATGAGGTTTGGGAGAAGTTT | |
| | GATCCCGATGCAACTCAGTTCATGGAATTTGAAAAATTATCTCAGTTT | |
| | GCAGCTGCGCTTGAACCGCCTCTCAATCTGCCACAACCAAACAAACTC | |
| | CAGCTCATTGCCATGGATTTGCCCATGGTGAGTGGTGACCGGATCCAC | |
| | TGTCTTGATATCTTATTTGCTTTTACAAAGCGGGTTCTAGGAGAGAGT | |
| | GGAGAGATGGATGCTCTACGAATACAGATGGAAGAGCGATTCATGGCT | |
| | TCCAATCCTTCCAAGGTCTCCTATCAGCCAATCACTACTACTTTAAAAC | |
| | GAAAACAAGAGGAAGTATCTGCTGTCATTATTCAGCGTGCTTACAGAC | |
| | GCCACCTTTTAAAGCGAACTGTAAAACAAGCTTCCTTTACGTACAATAA | |
| | AAACAAAATCAAAGGTGGGGCTAATCTTCTTATAAAAGAAGACATGAT | |
| | AATTGACAGAATAAATGAAAACTCTATTACAGAAAAAACTGATCTGACC | |
| | ATGTCCACTGCAGCTTGTCCACCTTCCTATGACCGGGTGACAAAGCCA | |
| | ATTGTGGAAAAACATGAGCAAGAAGGCAAAGATGAAAAAGCCAAAGGG | |
| | AAATAA | |
| 56 | MEQTVLVPPGPDSFNFFTRESLAAIERRIAEEKAKNPKPDKKDDDENGPK | hNAv1.1-amino acid |
| | PNSDLEAGKNLPFIYGDIPPEMVSEPLEDLDPYYINKKTFIVLNKGKAIFRF | NP_001189364 |
| | SATSALYILTPFNPLRKIAIKILVHSLFSMLIMCTILTNCVFMTMSNPPDWT | |
| | KNVEYTFTGIYTFESLIKIIARGFCLEDFTFLRDPWNWLDFTVITFAYVTEF | |
| | VDLGNVSALRTFRVLRALKTISVIPGLKTIVGALIQSVKKLSDVMILTVFCL | |
| | SVFALIGLQLFMGNLRNKCIQWPPTNASLEEHSIEKNMNYNGTLINETV | |
| | FEFDWKSYIQDSRYHYFLEGFLDALLCGNSSDAGQCPEGYMCVKAGRNP | |
| | NYGYTSFDTFSWAFLSLFRLMTQDFWENLYQLTLRAAGKTYMIFFVLVIF | |
| | LGSFYLINLILAVVAMAYEEQNQATLEEAEQKEAEFQQMIEQLKKQQEAA | |
| | QQAATATASEHSREPSAAGRLSDSSSEASKLSSKSAKERRNRRKKRKQKE | |
| | QSGGEEKDEDEFQKSESEDSIRRKGFRFSIEGNRLTYEKRYSSPHQSLLSI | |
| | RGSLFSPRRNSRTSLFSFRGRAKDVGSENDFADDEHSTFEDNESRRDSLF | |
| | VPRRHGERRNSNLSQTSRSSRMLAVFPANGKMHSTVDCNGVVSLVGGP | |
| | SVPTSPVGQLLPEVIIDKPATDDNGIIIETEMRKRRSSSFHVSMDFLEDP | |
| | SQRQRAMSTASILTNITVEELEESRQKCPPCWYKFSNIFLIWDCSPYWLKV | |
| | KHVVNLWMDPFVDLAITICIVLNTLFMAMEHYPMTDHFNNM:TVGNLVF | |
| | TGIFTAEMFLKIIAMDPYYYFQEGWNIFDGFIVTLSLVELGLANVEGLSVL | |
| | RSFRLLRVFKLAKSWPTLNMLIKIIGNSVGALGNLTLVLAIIVFIFAVVGMQ | |
| | LFGKSYKDCVCKIASDCQLPRWHMNDFFHSFLIVFRVLCGEWIETMWDC | |
| | MEVAGQAMCLTVFMMVMVIGNLVVLNLFLALLLSSFSADNLAATDDDNE | |
| | MNNLQIAVDRMHKGVAYVKRKIYEFIQQSFIRKQKILDEIKPLDDLNNKK | |
| | DSCMSNHTAEIGKDLDYLKDVNGTTSGIGTGSSVEKYIIDESDYMSFINN | |
| | PSLIVTVPIAVGESDFENLNTEDFSSESDLEESKEKLNESSSSSEGSTVDI | |
| | GAPVEEQPVVEPEETLEPEACFTEGCVQRFKCCQINVEEGRGKQWVVNLR | |
| | RTCFRIVEHNWFETFIVFMILLSSGALAFEDIYIDQRKTIKTMLEYADKVF | |
| | TYIFILEMLLKWVAYGYQTYFTNAWCWLDFLIVDVSLVSLTANALGYSE1 | |
| | GAIKSLRTLRALRPLRALSRFEGMRVVVNALLGAIPSIMNVLLVCLIFWLIF | |
| | SIMGVNLFAGKFYHCINTTTGDRFDIEDVNNHTDCLKLIERNETARWKN | |
| | VINNFDNVGFGYLSLLQVATFKGWMDIMYAAVDSRNVEIQPKYEESLYM | |
| | YLYFVIFIIFGSFFTLNLFIGVIIDNFNQQKKKFGGQDIFMTEEQKKYYNA | |
| | MKKLGSKKPQKPIPRPGNKFQGMVFDFVTRQVFDISIMILICLNMVTMMV | |
| | ETDDQSEYVTTILSRINLVFIVLFTGECVLKLISLRHYYFTIGWNIFDFVVV | |
| | ILSIVGMFLAEIIEKYFVSPTLFRVIRLARIGRILRLIKGAKGIRTLLFALMM | |
| | SLPALFNIGLLLFLVMFIYAIFGMSNFAYVKREVGIDDMFNFETFGNSMIC | |
| | LFQITTSAGWDGLLAPILNSKPPDCDPNWNPGSSVKGDCGNPSVGIFFF | |
| | VSYIIISFLVVVNMYIAVILENFSVATEESAEPLSEDDFEMFYEVWEKFDPD | |
| | ATQFMEFEKLSQFAAALEPPLNLPQPNKLQLIAMDLPMVSGDRIHCLDILF | |
| | AFTKRVLGESGEMDALRIQMEERFMASNPSKVSYQPITTTLKRKQEEVSA | |
| | VIIQRAYRRHLLKRIVKQASFTYNKNKIKGGANLLIKEDMIIDRINENSTTE | |
| | KTDLTMSTAACPPSYDRVTKPIVEKHEQEGKDEKAKGK | |
| 57 | FMTMSNPP | hNAv1.1D1 E1 loop |
| 58 | VTEFVDLGN | hNAv1.1&1.2&1.3D1 |
| | | E2 loop |
| 59 | MGNLRNKCIQWPPTNASLEEHSIEKNITVNYNGTLINETVFEFDWKSYIQ | hNAv1.1D1 E3 loop |
| | DSRYHYFLEGFLDALLCGNSSDAGQCPEGYMCVKAGRNPNYGYTSFDTF | |
| | SWAFLSLFRLMTQDFWENLYQLTLRAAGK | |
| 60 | TEFVNLGNVSALRT | Residues 202-215 of |
| | | NAV1.7 |
| 61 | MAMEHYPMTDHFN | hNAv1.1D2 E1 loop |
| 62 | VEIGLANVEG | hNAv1.1D2 E2 loop |
| 63 | GKSYKDCVCKIASDCQLPRWHMNDFFHSFLIVFRVLCGEWIETMWDCM | hNAv1.1D2 E3 loop |
| | EVAGQA | |
| 64 | TEFVNLGNVSALRTFRVLRALKTISVIPGLK | Residues 202-232 of |
| | | NAV1.7 |
| 65 | LAFEDIYIDQRKTIK | hNAv1.1D3 E1 loop |
| 66 | VSLTANALGYSE1GAIK | hNAv1.1&1.2D3 E2 |
| | | loop |
| 67 | AGKFYHCINTTTGDRFDIEDVNNHTDCLKLIERNETARWKNVKVNFDNV | hNAv1.1D3 E3 loop |
| | GFGYLSLLQVATFKGWMDIMYAAVDSRNVE1QPKYEESL | |
| 68 | NLGNVS | Residues 206-211 of |
| | | NAV1.7 |
| 69 | MMVETDDQSEYVTT | hNAv1.1D4 E1 loop |
| 70 | VGMFLAEIIEKYFVSPTLFR | hNAv1.1&1.2D4 E2 |
| | | loop |
| 71 | AYVKREVGIDDMFNFETFGNSMICLFQITTSAGWDGLLAPILNSKPPDCD | hNAv1.1D4 E3 loop |
| | PNKVNPGSSVKGDCGNPS | |
| 72 | MAMLPPPGPQSFVHFTKQSLALIEQRISEEKAKGHKDEKKDDEEEGPKPS | Mouse NAV1. 7 |
| | SDLEAGKQLPFIYGDIPPGMVSEPLEDLDPYYADKKTFIVLNKGKAIFRFN | Mus musculus |
| | ATPALYMLSPFSPLRRISIKILVHSLFSMLIMCTILTNCIFMTMSNPPDVNTK | NP_001277603 |
| | NVEYTFTGIYTFESLIKILARGFCVGEFTFLRDPWNWLDFVVIVFAYLTEF | |
| | VNLGNVSALRTFRVLRALKTISVIPGLKTIVGALIQSVKKLSDVMILTVFCL | |
| | SVFALIGLQLFMGNLKHKCFRKDLEQNETLESIMSTAESEEE1KRYFYYLE | |
| | GSKDALLCGFSTDSGQCPEGYECITTAGRNPDYGYTSFDTFGWAFLALFR | |
| | LMTQDYWENLYQQTLRAAGKTYMIFFVVVIFLGSFYLINLILAVVAMAYE | |
| | EQNQANIEEAKQKE1EFQQMLDRLKKEQEEAEAIAAAAAEYTSLGRSRIM | |
| | GLSESSSETSRLSSKSAKERRNRRKKKKQKLSSGEEKGDDEKLSKSGSEE | |
| | SIRKKSFHLGVEGHHRAREKRLSTPNQSPLSIRGSLFSARRSSRTSLFSFK | |
| | GRGRDLGSETEFADDEHSIFGDNESRRGSLFVPHRPRERRSSNISQASRS | |
| | PPVLPVNGKMHSAVDCNGVVSLVDGPSALMLPNGQLLPEVIIDKATSDDS | |
| | GTTNQMRKKRLSSSYFLSEDMLNDPHLRQRAMSRASILTNTNEELEESR | |
| | QKCPPWWYRFAHTFLIWNCSPYVVIKFKKFIYFIVMDPFVDLAMCIVLNT | |
| | LFMAMEHHPMTDEFKNVLAVGNLVFTGIFAAEMVLKLIAMDPYEYFQVG | |
| | WNIFDSLIVTLSLVE1FLADVEGLSVLRSFRLLRVFKLAKSWPTLNMLIKII | |
| | GNSVGALGNLTLVLAIIVFIFAVVGMQLFGKSYKECVCKINENCKLPRWH | |
| | MNDFFHSFLIVFRVLCGEWIETMWDCMEVAGQTMCLIVYMMVMVIGNL | |
| | VVLNLFLALLLSSFSSDNLTAIEEDTDANNLQIAVARIKRGINYVKQTLREF | |
| | ILKSFSKKPKGSKDTKRTADPNNKRENYISNRTLAEISKDHNFLKEKDKIS | |
| | GFSSSLDKSFMDENDYQSFIHNPSLIVTVPIAPGESDLENMNTEEISSDS | |
| | DSDYSKERRNRSSSSECSTVDNPLPGEEEAEAEPINADEPEACFTDGCVR | |
| | RFPCCQVNIDSGKGKVWWTIRKTCYRIVEHSWFESFIVLMILLSSGALAF | |
| | EDIYIEKKKTIKIILEYADKIFTYIFILEMLLKIMAYGYKTYFTNAWCVVLDF | |
| | LIVDVSLVTLVANTLGYSDLGPIKSLRTLRALRPLRALSRFEGMRVVVNALI | |
| | GAIPSIMNVLLVCLIFWLIFSIMGVNLFAGKFYECVNTTDGSRFSVSQVAN | |
| | RSECFALMNVSGNVRWKNLINNFDNVGLGYLSLLQVATFKGWMDIMYA | |
| | AVDSVNVNAQPIYEYNLYMYIYFVIFIIFGSFFTLNLFIGVIIDNFNQQKKK | |
| | LGGQDIFMTEEQKKYYNAMKKLGSKKPQKPIPRPGNKFQGCIFDLVTNQ | |
| | AFDITIMVLICLNMVTMMVEKEGQTDYMSFVLWVNVVFIILFTGECVLKL | |
| | ISLRHYYFTVGWNIFDFVVVILSIVGMFLAEMIEKYFVSPTLFRVIRLARIG | |
| | RILRLIKGAKGIRTLLFALMMSLPALFNIGLLLFLVMFIYAIFGMSNFAYVKK | |
| | EAGINDMFNFETFGNSMICLFQITTSAGWDGLLAPILNSAPPDCDPKKVH | |
| | PGSSVEGDCGNPSVGIFYFVSYIIISFLVVVNMYIAVILENFSVATEESTEP | |
| | LSEDDFEMFYEVWEKFDPDATQFIEFCKLSDFAAALDPPLLIAKPNKVQLI | |
| | AMDLPMVSGDRIHCLDILFAFTKRVLGESGEMDSLRSQMEERFMSANPS | |
| | KVSYEPITTTLKRKQEDVSATIIQRAYRRYRLRQNVKNISSIYIKDGDRDD | |
| | DLPNKEDIVFDNVNENSSPEKTDATASTISPPSYDSVTKP | |
| | DQEKYETDKTEKEDKEKDESRK | |
| 73 | ATGCTGTTTTCTAACAGACATTGGGTACCATCGAATGACTGTCAGAAC | hNAv1.2-nucleotide |
| | AGAAAGCTAAGGCAAAGGAGGGAGGATGCTGTGGTCATCCTTTCTTGT | NM_001040142 |
| | TTTTTTCTTCTTTAATGAGGATAGAGCACATGTGAGATTTTACTTTCTA | |
| | CTCCAGTAAAAATTCTGAAGAATTGCATTGGAGACTGTTATATTCAACA | |
| | CATACGTGGATTCTGTGTTATGATTTACATTTTCTTTATTTCAGCACT | |
| | TTCTTATGCAAGGAGCTAAACAGTGATTAAAGGAGCAGGATGAAAAGA | |
| | TGGCACAGTCAGTGCTGGTACCGCCAGGACCTGACAGCTTCCGCTTCT | |
| | TTACCAGGGAATCCCTTGCTGCTATTGAACAACGCATTGCAGAAGAGA | |
| | AAGCTAAGAGACCCAAACAGGAACGCAAGGATGAGGATGATGAAAATG | |
| | GCCCAAAGCCAAACAGTGACTTGGAAGCAGGAAAATCTCTTCCATTTA | |
| | TTTATGGAGACATTCCTCCAGAGATGGTGTCAGTGCCCCTGGAGGATC | |
| | TGGACCCCTACTATATCAATAAGAAAACGTTTATAGTATTGAATAAAGG | |
| | GAAAGCAATCTCTCGATTCAGTGCCACCCCTGCCCTTTACATTTTAACT | |
| | CCCTTCAACCCTATTAGAAAATTAGCTATTAAGATTTTGGTACATTCTT | |
| | TATTCAATATGCTCATTATGTGCACGATTCTTACCAACTGTGTATTTAT | |
| | GACCATGAGTAACCCTCCAGACTGGACAAAGAATGTGGAGTATACCTT | |
| | TACAGGAATTTATACTTTTGAATCACTTATTAAAATACTTGCAAGGGGC | |
| | TTTTGTTTAGAAGATTTCACATTTTTACGGGATCCATGGAATTGGTTG | |
| | GATTTCACAGTCATTACTTTTGCATATGTGACAGAGTTTGTGGACCTG | |
| | GGCAATGTCTCAGCGTTGAGAACATTCAGAGTTCTCCGAGCATTGAAA | |
| | ACAATTTCAGTCATTCCAGGCCTGAAGACCATTGTGGGGGCCCTGATC | |
| | CAGTCAGTGAAGAAGCTTTCTGATGTCATGATCTTGACTGTGTTCTGT | |
| | CTAAGCGTGTTTGCGCTAATAGGATTGCAGTTGTTCATGGGCAACCTA | |
| | CGAAATAAATGTTTGCAATGGCCTCCAGATAATTCTTCCTTTGAAATAA | |
| | ATATCACTTCCTTCTTTAACAATTCATTGGATGGGAATGGTACTACTTT | |
| | CAATAGGACAGTGAGCATATTTAACTGGGATGAATATATTGAGGATAA | |
| | AAGTCACTTTTATTTTTTAGAGGGGCAAAATGATGCTCTGCTTTGTGG | |
| | CAACAGCTCAGATGCAGGCCAGTGTCCTGAAGGATACATCTGTGTGAA | |
| | GGCTGGTAGAAACCCCAACTATGGCTACACGAGCTTTGACACTTTTAG | |
| | TTGGGCCTTTtTGTCCTTATTTCGTCTCATGACTCAAGACTTCTGGGA | |
| | AAACCTTTATCAACTGACACTACGTGCTGCTGGGAAAACGTACATGAT | |
| | ATTTTTTGTGCTGGTCATTTTCTTGGGCTCATTCTATCTAATAAATTTG | |
| | ATCTTGGCTGTGGTGGCCATGGCCTATGAGGAACAGAATCAGGCCACA | |
| | TTGGAAGAGGCTGAACAGAAGGAAGCTGAATTTCAGCAGATGCTCGAA | |
| | CAGTTGAAAAAGCAACAAGAAGAAGCTCAGGCGGCAGCTGCAGCCGCA | |
| | TCTGCTGAATCAAGAGACTTCAGTGGTGCTGGTGGGATAGGAGTTTTT | |
| | TCAGAGAGTTCTTCAGTAGCATCTAAGTTGAGCTCCAAAAGTGAAAAA | |
| | GAGCTGAAAAACAGAAGAAAGAAAAAGAAACAGAAAGAACAGTCTGGA | |
| | GAAGAAGAGAAAAATGACAGAGTCCGAAAATCGGAATCTGAAGACAGC | |
| | ATAAGAAGAAAAGGTTTCCGTTTTTCCTTGGAAGGAAGTAGGCTGACA | |
| | TATGAAAAGAGATTTTCTTCTCCACACCAGTCCTTACTGAGCATCCGTG | |
| | GCTCCCTTTTCTCTCCAAGACGCAACAGTAGGGCGAGCCTTTTCAGCT | |
| | TCAGAGGTCGAGCAAAGGACATTGGCTCTGAGAATGACTTTGCTGATG | |
| | ATGAGCACAGCACCTTTGAGGACAATGACAGCCGAAGAGACTCTCTGT | |
| | TCGTGCCGCACAGACATGGAGAACGGCGCCACAGCAATGTCAGCCAGG | |
| | CCAGCCGTGCCTCCAGGGTGCTCCCCATCCTGCCCATGAATGGGAAGA | |
| | TGCATAGCGCTGTGGACTGCAATGGTGTGGTCTCCCTGGTCGGGGGC | |
| | CCTTCTACCCTCACATCTGCTGGGCAGCTCCTACCAGAGGGCACAACT | |
| | ACTGAAACAGAAATAAGAAAGAGACGGTCCAGTTCTTATCATGTTTCC | |
| | ATGGATTTATTGGAAGATCCTACATCAAGGCAAAGAGCAATGAGTATA | |
| | GCCAGTATTTTGACCAACACCATGGAAGAACTTGAAGAATCCAGACAG | |
| | AAATGCCCACCATGCTGGTATAAATTTGCTAATATGTGTTTGATTTGG | |
| | GACTGTTGTAAACCATGGTTAAAGGTGAAACACCTTGTCAACCTGGTT | |
| | GTAATGGACCCATTTGTTGACCTGGCCATCACCATCTGCATTGTCTTA | |
| | AATACACTCTTCATGGCTATGGAGCACTATCCCATGACGGAGCAGTTC | |
| | AGCAGTGTACTGTCTGTTGGAAACCTGGTCTTCACAGGGATCTTCACA | |
| | GCAGAAATGTTTCTCAAGATAATTGCCATGGATCCATATTATTACTTTC | |
| | AAGAAGGCTGGAATATTTTTGATGGTTTTATTGTGAGCCTTAGTTTAA | |
| | TGGAACTTGGTTTGGCAAATGTGGAAGGATTGTCAGTTCTCCGATCAT | |
| | TCCGGCTGCTCCGAGTTTTCAAGTTGGCAAAATCTTGGCCAACTCTAA | |
| | ATATGCTAATTAAGATCATTGGCAATTCTGTGGGGGCTCTAGGAAACC | |
| | TCACAAAGGTATTGGCCATCATCGTCTTCATTGCTGTGGAAAGTTGCA | |
| | TGCAGCTCTTTGGTAAGAGCTACAAAGAATGTGTCTGCAAGATTTCCA | |
| | ATGATTGTGAACTCCCACGCTGGCACATGCATGACTTTTTCCACTCCTT | |
| | CCTGATCGTGTTCCGCGTGCTGTGTGGAGAGTGGATAGAGACCATGT | |
| | GGGACTGTATGGAGGTCGCTGGCCAAACCATGTGCCTTACTGTCTTCA | |
| | TGATGGTCATGGTGATTGGAAATCTAGTGGTTCTGAACCTCTTCTTGG | |
| | CCTTGCTTTTGAGTTCCTTCAGTTCTGACAATCTTGCTGCCACTGATGA | |
| | TGATAACGAAATGAATAATCTCCAGATTGCTGTGGGAAGGATGCAGAA | |
| | AGGAATCGATTTGTTAAAAGAAAAATACGTGAATTTATTCAGAAAGC | |
| | CTTTGTTAGGAAGCAGAAAGCTTTAGATGAAATTAAACCGCTTGAAGA | |
| | TCTAAATAATAAAAAAGACAGCTGTATTTCCAACCATACCACCATAGAA | |
| | ATAGGCAAAGACCTCAATTATCTCAAAGACGGAAATGGAACTACTAGT | |
| | GGCATAGGCAGCAGTGTAGAAAAATATGTCGTGGATGAAAGTGATTAC | |
| | ATGTCATTTATAAACAACCCTAGCCTCACTGTGACAGTACCAATTGCTG | |
| | TTGGAGAATCTGACTTTGAAAATTTAAATACTGAAGAATTCAGCAGCG | |
| | AGTCAGATATGGAGGAAAGCAAAGAGAAGCTAAATGCAACTAGTTCAT | |
| | CTGAAGGCAGCACGGTTGATATTGGAGCTCCCGCCGAGGGAGAACAG | |
| | CCTGAGGTTGAACCTGAGGAATCCCTTGAACCTGAAGCCTGTTTTACA | |
| | GAAGACTGTGTACGGAAGTTCAAGTGTTGTCAGATAAGCATAGAAGAA | |
| | GGCAAAGGGAAACTCTGGTGGAATTTGAGGAAAACATGCTATAAGATA | |
| | GTGGAGCACAATTGGTTCGAAACCTTCATTGTCTTCATGATTCTGCTG | |
| | AGCAGTGGGGCTCTGGCCTTTGAAGATATATACATTGAGCAGCGAAAA | |
| | ACCATTAAGACCATGTTAGAATATGCTGACAAGGTTTTCACTTACATAT | |
| | TCATTCTGGAAATGCTGCTAAAGTGGGTTGCATATGGTTTTCAAGTGT | |
| | ATTTTACCAATGCCTGGTGCTGGCTAGACTTCCTGATTGTTGATGTCT | |
| | CACTGGTTAGCTTAACTGCAAATGCCTTGGGTTACTCAGAACTTGGTG | |
| | CCATCAAATCCCTCAGAACACTAAGAGCTCTGAGGCCACTGAGAGCTT | |
| | TGTCCCGGTTTGAAGGAATGAGGGTTGTTGTAAATGCTCTTTTAGGAG | |
| | CCATTCCATCTATCATGAATGTACTTCTGGTTTGTCTGATCTTTTGGCT | |
| | AATATTCAGTATCATGGGAGTGAATCTCTTTGCTGGCAAGTTTTACCA | |
| | TTGTATTAATTACACCACTGGAGAGATGTTTGATGTAAGCGTGGTCAA | |
| | CAACTACAGTGAGTGCAAAGCTCTCATTGAGAGCAATCAAACTGCCAG | |
| | GTGGAAAAATGTGAAAGTAAATTTTGATAACGTAGGACTTGGATATCT | |
| | GTCTCTACTTCAAGTAGCCACGTTTAAGGGATGGATGGATATTATGTA | |
| | TGCAGCTGTTGATTCACGAAATGTAGAATTACAACCCAAGTATGAAGA | |
| | CAACCTGTACATGTATCTTTATTTTGTCATCTTTATTATTTTTGGTTCA | |
| | TTCTTTACCTTGAATCTTTTCATTGGTGTCATCATAGATAACTTCAACC | |
| | AACAGAAAAAGAAGTTTGGAGGTCAAGACATTTTTATGACAGAAGAAC | |
| | AGAAGAAATACTACAATGCAATGAAAAAACTGGGTTCAAAGAAACCAC | |
| | AAAAACCCATACCTCGACCTGCTAACAAATTCCAAGGAATGGTCTTTGA | |
| | TTTTGTAACCAAACAAGTCTTTGATATCAGCATCATGATCCTCATCTGC | |
| | CTTAACATGGTCACCATGATGGTGGAAACCGATGACCAGAGTCAAGAA | |
| | ATGACAAACATTCTGTACTGGATTAATCTGGTGTTTATTGTTCTGTTCA | |
| | CTGGAGAATGTGTGCTGAAACTGATCTCTCTTCGTTACTACTATTTCAC | |
| | TATTGGATGGAATATTTTTGATTAAAAAAAAAAAAAAAAAAAAAAAATT | |
| | AGGAATGTTTCTGGCTGAACTGATAGAAAAGTATTTTGTGTCCCCTAC | |
| | CCTGTTCCGAGTGATCCGTCTTGCCAGGATTGGCCGAATCCTACGTCT | |
| | GATCAAAGGAGCAAAGGGGATCCGCACGCTGCTCTTTGCTTTGATGAT | |
| | GTCCCTTCCTGCGTTGTTTAACATCGGCCTCCTTCTTTTCCTGGTCATG | |
| | TTCATCTACGCCATCTTTGGGATGTCCAATTTTGCCTATGTTAAGAGG | |
| | GAAGTTGGGATCGATGACATGTTCAACTTTGAGACCTTTGGCAACAGC | |
| | ATGATCTGCCTGTTCCAAATTACAACCTCTGCTGGCTGGGATGGATTG | |
| | CTAGCACCTATTCTTAATAGTGGACCTCCAGACTGTGACCCTGACAAA | |
| | GATCACCCTGGAAGCTCAGTTAAAGGAGACTGTGGGAACCCATCTGTT | |
| | GGGATTTTCTTTTTTGTCAGTTACATCATCATATCCTTCCTGGTTGTGG | |
| | TGAACATGTACATCGCGGTCATCCTGGAGAACTTCAGTGTTGCTACTG | |
| | AAGAAAGTGCAGAGCCTCTGAGTGAGGATGACTTTGAGATGTTCTATG | |
| | AGGTTTGGGAGAAGTTTGATCCCGATGCGACCCAGTTTATAGAGTTTG | |
| | CCAAACTTTCTGATTTTGCAGATGCCCTGGATCCTCCTCTTCTCATAGC | |
| | AAAACCCAACAAAGTCCAGCTCATTGCCATGGATCTGCCCATGGTGAG | |
| | TGGTGACCGGATCCACTGTCTTGACATCTTATTTATTTTACAAAGCG | |
| | TGTTTTGGGTGAGAGTGGAGAGATGGATGCCCTTCGAATACAGATGG | |
| | AAGAGCGATTCATGGCATCAAACCCCTCCAAAGTCTCTTATGAGCCCA | |
| | TTACGACCACGTTGAAACGCAAACAAGAGGAGGTGTCTGCTATTATTA | |
| | TCCAGAGGGCTTACAGACGCTACCTCTTGAAGCAAAAAGTTAAAAAGG | |
| | TATCAAGTATATACAAGAAAGACAAAGGCAAAGAATGTGATGGAACAC | |
| | CCATCAAAGAAGATACTCTCATTGATAAACTGAATGAGAATTCAACTCC | |
| | AGAGAAAACCGATATGACGCCTTCCACCACGTCTCCACCCTCGTATGA | |
| | TAGTGTGACCAAACCAGAAAAAGAAAAATTTGAAAAAGACAAATCAGA | |
| | AAAGGAAGACAAAGGGAAAGATATCAGGGAAAGTAAAAAGTAAAAAGA | |
| | AACCAAGAATTTTCCATTTETGTGATCAATTGTTTACAGCCCGTGATGGT | |
| | GATGTGTTTGTGTCAACAGGACTCCCACAGGAGGTCTATGCCAAACTG | |
| | ACTGTTTTTACAAATGTATACTTAAGGTCAGTGCCTATAACAAGACAGA | |
| | GACCTCTGGTCAGCAAACTGGAACTCAGTAAACTGGAGAAATAGTATC | |
| | GATGGGAGGTTTCTATTTTCACAACCAGCTGACACTGCTGAAGAGCAG | |
| | AGGCGTAATGGCTACTCAGACGATAGGAACCAATTTAAAGGGGGGAG | |
| | GGAAGTTAAATTTTTATGTAAATTCAACATGTGACACTTGATAATAGTA | |
| | ATTGTCACCAGTGTTTATGTTTTAACTGCCACACCTGCCATATTTTTAC | |
| | AAAACGTGTGCTGTGAATTTATCACTTTTCTTTTTAATTCACAGGTTGT | |
| | TTACTATTATATGTGACTATTTTTGTAAATGGGTTTGTGTTTGGGGAG | |
| | AGGGATTAAAGGGAGGGAATTCTACATTTCTCTATTGTATTGTATAAC | |
| | TGGATATATTTTAAATGGAGGCATGCTGCAATTCTCATTCACACATAAA | |
| | AAAATCACATCACAAAAGGGAAGAGTTTACTTCTTGTTTCAGGATGTT | |
| | TTTAGATTTTTGAGGTGCTTAAATAGCTATTCGTATTTTTAAGGTGTCT | |
| | CATCCAGAAAAAATTTAATGTGCCTGTAAATGTTCCATAGAATCACAAG | |
| | CATTAAAGAGTTGTTTTATTTTTACATAACCCATTAAATGTACATGTAT | |
| | ATATGTATATATGTATATGTGCGTGTATATACATATATATGTATACACA | |
| | CATGCACACACAGAGATATACACATACCATTACATTGTCATTCACAGTC | |
| | CCAGCAGCATGACTATCACATTTTTGATAAGTGTCCTTTGGCATAAAAT | |
| | AAAAATATCCTATCAGTCCTTTCTAAGAAGCCTGAATTGACCAAAAAAC | |
| | ATCCCCACCACCACTTTATAAAGTTGATTCTGCTTTATCCTGCAGTATT | |
| | GTTTAGCCATCTTCTGCTCTTGGTAAGGTTGACATAGTATATGTCAAT | |
| | TTAAAAAATAAAAGTCTGCTTTGTAAATAGTAATTTTACCCAGTGGTGC | |
| | ATGTTTGAGCAAACAAAAATGATGATTTAAGCACACTACTTATTGCATC | |
| | AAATATGTACCACAGTAAGTATAGTTTGCAAGCTTTCAACAGGTAATAT | |
| | GATGTAATTGGTTCCATTATAGTTTGAAGCTGTCACTGCTGCATTTTT | |
| | ATCTTGCCTATGCTGCTGTATCTTATTCCTTCCACTGTTCAGAAGTCTA | |
| | ATATGGGAAGCCATATATCAGTGGTAAAGTGAAGCAAATTGTTCTACC | |
| | AAGACCTCATTCTTCATGTCATTAAGCAATAGGTTGCAGCAAACAAGG | |
| | AAGAGCTTCTTATTTTTATTCTTCCAACCTTAATTGAACACTCAATGA | |
| | TGAAAAGCCCGACTGTACAAACATGTTGCAAGCTGCTTAAATCTGTTT | |
| | AAAATATATGGTTAGAGTTTTCTAAGAAAATATAAATACTGTAAAAAGT | |
| | TCATTTTATTTTATTTTTCAGCCTTTTGTACGTAAAATGAGAAATTAAA | |
| | AGTATCTTCAGGTGGATGTCACAGTCACTATTGTTAGTTTCTGTTCCT | |
| | AGCACTTTTAAATTGAAGCACTTCACAAAATAAGAAGCAAGGACTAGG | |
| | ATGCAGTGTAGGTTTCTGCTTTTTTATTAGTACTGTAAACTTGCACACA | |
| | TTTCAATGTGAAACAAATCTCAAACTGAGTTCAAThTTTATTTGCTTTC | |
| | AATAGTAATGCCTTATCATTGAAAGAGGCTTAAAGAAAAAAAAAATCA | |
| | GCTGATACTCTTGGCATTGCTTGAATCCAATGTTTCCACCTAGTCTTTT | |
| | TATTCAGTAATCATCAGTTTTTTCCAATGTTTGTTTACACAGATAGATC | |
| | TTATTGACCCATATGGCACTAGAACTGTATCAGATATAATATGGGATC | |
| | CCAGCTTTTTTTCCTCTCCCACAAAACCAGGTAGTGAAGTTATATTACC | |
| | AGTTACAGCAAAATATTTTGTGTTTCACAAGCAACAATAAATGTAGATT | |
| | TTTATACTGAAGCTATTGACTTGTAGTGTGTTGGTGAAATGCATGCA | |
| | GGAAAATGCTGTTACCATAAAGAACGGTAAACCACATTACAATCAAGC | |
| | CAAAAGAATAAAGGTTTCATTTTGTTTTTGTATTTAATTGTTGTOTT | |
| | GTTTCTATCTTTGAAATGCCATTTAAAGGTAGATTTCTATCATGTAAAA | |
| | ATAATCTATCTGAAAAACAAATGTAAAGAACACACATTAATTACTATAA | |
| | TTCATCTTTCAATTTTTTCATGGAATGGAAGTTAATTAAGAAGAGTGTA | |
| | TTGGATAACTACTTTAATATTGGCCAAAAAGCTAGATATGGCATCAGG | |
| | TAGACTAGTGGAAAGTTACAAAAATTAATAAAAAATTGACTAACATur | |
| | AAAAAAAAAAAAAAAAAA | |
| 74 | MAQSVLVPPGPDSFRFFTRESLAAIEQRIAEEKAKRPKQERKDEDDENGP | hNAv1.2amino acid |
| | KPNSDLEAGKSLPFIYGDIPPEMVSVPLEDLDPYYINKKTFIVLNKGKAISR | NP_001035232 |
| | FSATPALYILTPFNPIRKLAIKILVHSLFNMLIMCTILTNCVFMTMSNPPDW | |
| | TKNVEYTFTGIYTFESLIKILARGFCLEDFTFLRDPWNWLDFTVITFAYVT | |
| | EFVDLGNVSALRTFRVLRALKTISVIPGLKTIVGALIQSVKKLSDVMILTVF | |
| | CLSVFALIGLQLFMGNLRNKCLQWPPDNSSFEINITSFFNNSLDGNGTIF | |
| | NRTVSIFNWDEYIEDKSHFYFLEGQNDALLCGNSSDAGQCPEGYICVKA | |
| | GRNPNYGYTSFDTFSWAFLSLFRLMTQDFWENLYQLTLRAAGKTYMIFF | |
| | VLVIFLGSFYLINLILAVVAMAYEEQNQATLEEAEQKEAEFQQMLEQLKKQ | |
| | QEEAQAAAAAASAESRDFSGAGGIGVFSESSSVASKLSSKSEKE1KNRRK | |
| | KKKQKEQSGEEEKNDRVRKSESEDSIRRKGFRFSLEGSRLTYEKRFSSPH | |
| | QSLLSIRGSLFSPRRNSRASLFSFRGRAKDIGSENDFADDEHSTFEDNDS | |
| | RRDSLFVPHRHGERRHSNVSQASRASRVLPILPMNGKMHSAVDCNGVVS | |
| | LVGGPSTLTSAGQLLPEGHIETEIRKRRSSSYHVSMDLLEDPTSRQRAM | |
| | SIASILTNTMEELEESRQKCPPCVVYKFANMCLIWDCCKPWLKVKHLVNL | |
| | VVMDPFVDLAITICIVLNTLFMAMEHYPMTEQFSSVLSVGNLVFTGIFTAE | |
| | MFLKIIAMDPYYYFQEGWNIFDGFIVSLSLMEIGLANVEGLSVLRSFRLLR | |
| | VFKLAKSWPTLNMLIKIIGNSVGALGNLTLVLAIIVFIFAVVGMQLFGKSYK | |
| | ECVCKISNDCEIPRWHMHDFFHSFLIVFRVLCGEWIETMWDCMEVAGQ | |
| | TMCLTVFMMVMVIGNLVVLNLFLALLLSSFSSDNLAATDDDNEMNNLQI | |
| | AVGRMQKGIDFVKRKIREFIQKAFVRKQKALDEIKPLEDLNNKKDSCISN | |
| | HTTIEIGKDLNYLKDGNGTTSGIGSSVEKYVVDESDYMSFINNPSLTVTV | |
| | PIAVGESDFENLNTEEFSSESDMEESKEKLNATSSSEGSTVDIGAPAEGE | |
| | QPEVEPEESLEPEACFTEDCVRKFKCCQISIEEGKGKLVVWNLRKTCYKIV | |
| | EHNWFETFIVFMILLSSGALAFEDIYIEQRKTIKTMLEYADKVFFYIFILEM | |
| | LLKWVAYGFQVYFTNAWCWLDFLIVDVSLVSLTANALGYSEIGAIKSLRT | |
| | LRALRPLRALSRFEGMRVVVNALLGAIPSIMNVLLVCLIFWLIFSIMGVNLF | |
| | AGKFYHCINYTTGEMFDVSVVNNYSECKALIESNQTARWKNVKVNFDNV | |
| | GLGYLSLLQVATFKGWMDIMYAAVDSRNVEIQPKYEDNLYMYLYFVIFII | |
| | FGSFFTLNLFIGVIIDNFNQQKKKFGGQDIFMTEEQKKYYNAMKKLGSKK | |
| | PQKPIPRPANKFQGMVFDFVTKQVFDISIMILICLNMVTMMVETDDQSQ | |
| | EMTNILYWINLVFIVLFTGECVLKLISLRYYYFFIGWNIFDFVVVILSIVGM | |
| | FLAE1IEKYFVSPTLFRVIRLARIGRILRLIKGAKGIRTLLFALMMSLPALFNI | |
| | GLLLFLVMFIYAIFGMSNFAYVKREVGIDDMFNFETFGNSMICLFQITTSA | |
| | GWDGLLAPILNSGPPDCDPDKDHPGSSVKGDCGNPSVGIFFFVSYIIISFL | |
| | VVVNMYIAVILENFSVATEESAEPLSEDDFEMFYEVWEKFDPDATQFIEF | |
| | AKLSDFADALDPPLLIAKPNKVQLIAMDLPMVSGDRIHCLDILFAFTKRVL | |
| | GESGEMDALRIQMEERFMASNPSINSYEPITTTLKRKQEEVSAIIIQRAYR | |
| | RYLLKQKVKKVSSIYKKDKGKECDGTPIKEDTLIDKLNENSTPEKTDMTPS | |
| | TTSPPSYDSVTKPEKEKFEKDKSEKEDKGKDIRESKK | |
| 75 | FMTMSNPP | hNAv1.2D1 E1 loop |
| 76 | VTEFVDLGN | hNAv1.2D1 E2 loop |
| 77 | MGNLRNKCLQWPPDNSSFEINITSFFNNSLDGNGTTFNRTVSIFNWDEY | hNAv1.2D1 E3 loop |
| | IEDKSHFYFLEGQNDALLCGNSSDAGQCPEGYICVKAGRNPNYGYTSFD | |
| | TFSWAFLSLFRLMTQDFWENLYQLTLRAAGK | |
| 78 | MEFPFGSVGTINFRRFTPESLAEIEKQIAAHRAAKKGRPKQRGQKDKSEK | Mouse NAV1.8 |
| | PRPQLDLKACNQLPRFYGEIPAEIVGEPLEDLDPFYSTHRTFIVLDKSRTI | Mus musculus |
| | SRFSATWALWLFSPFNLIRRTAIKVSVHSWFSIFITVTILVNCVCMTRIDL | NP_001192250 |
| | PEKLEYAFTVVYTFEALIKILARGFCLNEFTYLRDPWNWLDFSVITLAYVG | |
| | AAIDLRGISGLRTFRVLRALKTVSVIPGLKWVGALIHSVRKLADVTILTVF | |
| | CLSVFALVGLQLFKGNLKNKCIKNGTDPHKADNLSSEMAGDIFIKPGIiD | |
| | PLLCGNGSDAGHCPNDYVCRKTSDNPDFNYTSFDSFAWAFLSLFRLMTQ | |
| | DSWERLYQQTLRASGKMYMVFFVLVIFLGSFYLVNLILAWTMAYEEQSQ | |
| | ATIAEIEAKEKKFKEALEVLQKEQEVLAALGIDTTSLYSHNGSPLAPKNAN | |
| | ERRPRVKSRMSEGSTDDNRSLQSDPYNQRRMSFLGLSSGRRRASHSSVF | |
| | HFRAPSQDVSFPDGILDDGVFHGDQESRRSSILLGRGAGQAGPLPRSPLP | |
| | QSPNPGPRRGEEGQRGVPTGEIATGAPEGPALDAAGQKNFLSADYLNEP | |
| | FRAQRAMSVVSIMTSVIEEIEESKLKCPPCLISLAQKYLIWECCPKWKKFK | |
| | MVLFEIVTDPFAEITITLCIVVNTVFMAMEHYPMTDAFDAMLQAGNIVFT | |
| | VFFTMEMAFKIIAFDPYYYFQKKWNIFDCVIVTVSLLEISTSKKGSLSVLR | |
| | TFRLLRVFKLAKSWPTLNMLIKIIGNSVGALGNLTFILAIIVFIFALVGKQLL | |
| | SENYGCRRDGISVWNGERLRWHMCDFFHSFLVVFRILCGEWIENMWVC | |
| | MEVSQDYICLTLFLTVMVLGNLWLNLFIALLLNSFSADNLTAPEDDGEVN | |
| | NLQVALARIQVFGHRASRAITSYIRSHCRLRWPKVETQLGMKPPLTSCKA | |
| | ENHIATDAVNAAVGNLAKPALGGPKENHGDFITDPNVWVSVPIAEGESD | |
| | LDE1EEDVEHASQSSWQEESPKGQQEILQQVQKCEDHQAARSPPSGMS | |
| | SEDLAPYLGERWQREESPRVPAEGVDDTSSSEGSTVDCPDPEEILRKIPE | |
| | LAEE1DEPDDCFPEGGRRCPCCKVNTSKFPWATGWQVRKTCYRIVEHS | |
| | WFESFIIFMILLSSGALAFEDNYLEEKPRVKSVLEYTDRVFTFIFVFEMLLK | |
| | WVAYGFKKYFTNAWCWLDFLIVNISLTSLIAKILEYSDVASIKALRTLRAL | |
| | RPLRALSRFEGMRVVVDALVGAIPSIMNVLLVCLIFWLIFSIMGVNLFAGK | |
| | FSRCVDTRSNPFSVVNSTFVTNKSDCYNQNNTGHFFWVNVKVNFDNVA | |
| | MGYLALLQVATFKGWMDIMYAAVDSRDINSQPNWEESLYMYLYFVVFII | |
| | FGGFFTLNLFVGVIIDNFNQQKKKIRGQDIFMTEEQKKYYNAMKKLGSKK | |
| | PQKPIPRPLNKYQGFVFDIVTRQAFDIIIMALICLNMITMMVETDNQSEEK | |
| | TKVLGRINQFFVAVFTGECVMKMFALRQYYFTNGWNVFDFIVVILSISSLL | |
| | FSAILSSLESYFSPTLLRVIRLARIGRILRLIRAAKGIRTLLFALMMSLPALFN | |
| | IGLLLFLVMFIYSIFGMASFANVIDEAGIDDMFNFKTFGNSMLCLFQITTS | |
| | AGWDGLLSPILNTGPPYCDPNRPNSNGSKGNCGSPAVGILFFTTYIIISFL | |
| | IVVNMYIAVILENFNVATEESTEPLSEDDFDMFYETVVEKFDPEATQFIAFS | |
| | ALSDFADTLSGPLRIPKPNQNILIQMDLPLVPGDKIHCLDILFAFTKNVLGE | |
| | SGE1DSLKTNMEEKFMATNLSKASYEPIATTLRCKQEDISATIIQKAYRNY | |
| | MLQRSLMLSNPLHVPRAEEDGVSLPREGYVTFMANDNGGLPDKSETASA | |
| | TSFPPSYDSVTRGLSDRANISTSSSMQNEDEVTAKEGKSPGPQ | |
| 79 | MAMEHYPMTEQFS | hNAv1.2-D2 E1 loop |
| 80 | MEIGLANVEG | hNAv1.2-D2 E2 loop |
| 81 | GKSYKECVCKISNDCEIPRWHMHFFHSFLIVFRVLCGEWIETMWDCM | hNAv1.2-D2 E3 loop |
| | EVAGQT | |
| 82 | MEERYYPVIFPDERNFRPFTFDSLAAIEKRMQKEKKKSKDKAATEPQPR | Mouse NAV1.9 |
| | PQLDLKASRKLPKLYGDVPPDLIAKPLEDLDPFYKDHKTFMVLNKKRTIYR | Mus musculus: |
| | FSAKRALFILGPFNPIRSFMIRISVHSVFSMFIICTVIINCMFMANNSSVDS | NP_036017 |
| | RPSSNIPEYVFIGIYVLEAVIKILARGFIVDEFSYLRDPWNWLDFIVIGTAI | |
| | APCFLGNKVNNLSTLRTFRVLRALKAISVISGLKVIVGALLRSVKKLVDVM | |
| | VLTLFCLSIFALVGQQLFMGILSQKCIKDDCGPNAFSNKDCFVKENDSED | |
| | FIMCGNWLGRRSCPDGSTCNKTTFNPDYNYTNFDSFGWSFLAMFRVMT | |
| | QDSWEKLYRQILRTSGIYFVFFFVWIFLGSFYLLNLTLAVVTMAYEEQNR | |
| | NVAAETEAKEKMFQEAQQLLREEKEALVAMGIDRTSLNSLQASSFSPKKR | |
| | KFFGSKTRKSFFMRGSKTARASASDSEDDASKNPQLLEQTKRLSQNLPVE | |
| | LFDEHVDPLHRQRALSAVSILTITMQEQEKSQEPCFPCGKNLASKYLVWE | |
| | CSPPWLCIKKVLQTIMTDPFTE1AITICIIVNTVFLAMEHHNMDNSLKDIL | |
| | KIGNWVFTGIFIAEMCLKIIALDPYHYFRHGWNIFDSIVALVSLADVLFHK | |
| | LSKNLSFLASLRVLRVFKLAKSWPTLNTLIKIIGHSVGALGNLTVVLTIVVFI | |
| | FSVVGMRLFGAKFNKTCSTSPESLRRWHMGDFYHSFLVVFRILCGEWIE | |
| | NMWECMQEMEGSPLCVIVFVLIMVVGKLVVLNLFIALLLNSFSNEEKDGN | |
| | PEGETRKTKVQLALDRFSRAFYFMARALQNFCCKRCRRQNSPKPNEATE | |
| | SFAGESRDTATLDTRSWKEYDSEMTLYTGQAGAPLAPLAKEEDDMECCG | |
| | ECDASPTSQPSEEAQACDLPLKTKRLPSPDDHGVEMEVFSEEDPNLTIQS | |
| | ARKKSDAASMLSECSTIDLNDIFRNLQKTVSPQKQPDRCFPKGLSCIFLCC | |
| | KTIKKKSPWVLWWNLRKTCYQIVKHSWFESFIIFVILLSSGALIFEDVNLP | |
| | SRPQVEKLLKCTDNIFFIFLLEMILKWVAFGFRKYFTSAWCWLDFLIVVV | |
| | SGLSLTNLPNLKSFRNLRALRPLRALSQFEGMKVVVNALMSAIPAILNVLL | |
| | VCLIFWLIFCILGVNFFSGKFGRCINGTDINKYFNASNVPNQSQCLVSNYT | |
| | WKWNVNFDNVGNAYLALLQVATYKGWLDIMNAAVDSRGKDEQPAFEA | |
| | NLYAYLYFVVFIIFGSFFTLNLFIGVIIDNFNQQQKKLGGQDIFMTEEQKK | |
| | YYNAMKKLGTKKPQKPIPRPLNKCQAFVFDLVTSQVFDVIILGLIVTNMII | |
| | MMAESEGQPNEVKKIFDILNIVFVVIFTVECLIKVFALRQHYFTNGWNLFD | |
| | CVVVVLSIISTLVSGLENSNVFPPTLFRIVRLARIGRILRLVRAARGIRTLLF | |
| | ALMMSLPSLFNIGLLLFLVMFIYAIFGMNWFSINKRGSGIDDIFNFDTFSG | |
| | SMLCLFQITTSAGWDALLNPMLESKASCNSSSQESCQQPQIAIVYFVSYII | |
| | ISFLIWNMYIAVILENFNTATEESEDPLGEDDFEIFYEIWEKFDPEATQFI | |
| | QYSSLSDFADALPEPLRVAKPNRFQFLMMDLPMVMGDRLHCMDVLFAFT | |
| | TRVLGNSSGLDTMKAMMEEKFMEANPFKKLYEPIVITTKRKEEEECAAVI | |
| | QRAYRRHMEKMIKLKLKGRSSSSLQVFCNGDLSSLDVPKIKVHCD | |
| 83 | LAFEDIYIEQRKTIK | hNAv1.2D3 E1 loop |
| 84 | VSLTANALGYSE1GAIK | hNAv1.2D3 E2 loop |
| 85 | AGKFYHCINYTTGEMFDVSVVNNYSECKALIESNQTARWKNVINNFDNV | |
| | GLGYLSLLQVATFKGWMDIMYAAVDSRNVEIQPKYEDNL | hNAv1.2D3 E3 loop |
| 86 | VSYIPG | hNAV1.9 motif |
| 87 | MMVETDDQSQEMTN | hNAv1.2D4 E1 loop |
| 88 | VGMFLAEIIEKYFVSPTLFR | hNAv1.2D4 E2 loop |
| 89 | AYVKREVGIDDMFNFETFGNSMICLFQITTSAGWDGLLAPILNSGPPDCD | |
| | PDKDHPGSSVKGDCGNPS | hNAv1.2D4 E3 loop |
| 90 | TMISTLEN | hNAV1.8 motif |
| 91 | ATGGCACAGGCACTGTTGGTACCCCCAGGACCTGAAAGCTTCCGCCTT | hNAv1.3-nucleotide |
| | TTTACTAGAGAATCTCTTGCTGCTATCGAAAAACGTGCTGCAGAAGAG | NM_006922 |
| | AAAGCCAAGAAGCCCAAAAAGGAACAAGATAATGATGATGAGAACAAA | |
| | CCAAAGCCAAATAGTGACTTGGAAGCTGGAAAGAACCTTCCATTTATT | |
| | TATGGAGACATTCCTCCAGAGATGGTGTCAGAGCCCCTGGAGGACCTG | |
| | GATCCCTACTATATCAATAAGAAAACTTTTATAGTAATGAATAAAGGAA | |
| | AGGCAATTTTCCGATTCAGTGCCACCTCTGCCTTGTATATTTTAACTCC | |
| | ACTAAACCCTGTTAGGAAAATTGCTATCAAGATTTTGGTACATTTGA | |
| | TTCAGCATGCTTATCATGTGCACTATTTTGACCAACTGTGTATTTATGA | |
| | CCTTGAGCAACCCTCCTGACTGGACAAAGAATGTAGAGTACACATTCA | |
| | CTGGAATCTATACCTTTGAGTCACTTATAAAAATCTTGGCAAGAGGGT | |
| | TTTGCTTAGAAGATTTACGTTTCTTCGTGATCCATGGAACTGGCTGG | |
| | ATTTCAGTGTCATTGTGATGGCATATGTGACAGAGTTTGTGGACCTGG | |
| | GCAATGTCTCAGCGTTGAGAACATTCAGAGTTCTCCGAGCACTGAAAA | |
| | CAATTTCAGTCATTCCAGGTTTAAAGACCATTGTGGGGGCCCTGATCC | |
| | AGTCGGTAAAGAAGCTTTCTGATGTGATGATCCTGACTGTGTTCTGTC | |
| | TGAGCGTGTTTGCTCTCATTGGGCTGCAGCTGTTCATGGGCAATCTGA | |
| | GGAATAAATGTTTGCAGTGGCCCCCAAGCGATTCTGCTTTTGAAACCA | |
| | ACACCACTTCCTACTTTAATGGCACAATGGATTCAAATGGGACATTTGT | |
| | TAATGTAACAATGAGCACATTTAACTGGAAGGATTACATTGGAGATGA | |
| | CAGTCACTTTTATGTTTTGGATGGGCAAAAAGACCCTTTACTCTGTGG | |
| | AAATGGCTCAGATGCAGGCCAGTGTCCAGAAGGATACATCTGTGTGAA | |
| | GGCTGGTCGAAACCCCAACTATGGCTACACAAGCTTTGACACCTTTAG | |
| | CTGGGCTTTCCTGTCTCTATTTCGACTCATGACTCAAGACTACTGGGA | |
| | AAATCTTTACCAGTTGACATTACGTGCTGCTGGGAAAACATACATGAT | |
| | ATTTTTTGTCCTGGTCATTTTCTTGGGCTCATTTTATTTGGTGAATTTG | |
| | ATCCTGGCTGTGGTGGCCATGGCCTATGAGGAGCAGAATCAGGCCACC | |
| | TTGGAAGAAGCAGAACAAAAAGAGGCCGAATTTCAGCAGATGCTCGAA | |
| | CAGCTTAAAAAGCAACAGGAAGAAGCTCAGGCAGTTGCGGCAGCATCA | |
| | GCTGCTTCAAGAGATTTCAGTGGAATAGGTGGGTTAGGAGAGCTGTT | |
| | GGAAAGTTCTTCAGAAGCATCAAAGTTGAGTTCCAAAAGTGCTAAAGA | |
| | ATGGAGGAACCGAAGGAAGAAAAGAAGACAGAGAGAGCACCTTGAAG | |
| | GAAACAACAAAGGAGAGAGAGACAGCTTTCCCAAATCCGAATCTGAAG | |
| | ACAGCGTCAAAAGAAGCAGCTTCCTTTTCTCCATGGATGGAAACAGAC | |
| | TGACCAGTGACAAAAAATTCTGCTCCCCTCATCAGTCTCTCTTGAGTAT | |
| | CCGTGGCTCCCTGTTTTCCCCAAGACGCAATAGCAAAACAAGCA | |
| | CAGTTTCAGAGGTCGGGCAAAGGATGTTGGATCTGAAAATGACTTTGC | |
| | TGATGATGAACACAGCACATTTGAAGACAGCGAAAGCAGGAGAGACTC | |
| | ACTGTTTGTGCCGCACAGACATGGAGAGCGACGCAACAGTAACGTTAG | |
| | TCAGGCCAGTATGTCATCCAGGATGGTGCCAGGGCTTCCAGCAAATGG | |
| | GAAGATGCACAGCACTGTGGATTGCAATGGTGTGGTTTCCTTGGTGG | |
| | GTGGACCTTCAGCTCTAACGTCACCTACTGGACAACTTCCCCCAGAGG | |
| | GCACCACCACAGAAACGGAAGTCAGAAAGAGAAGGTTAAGCTCTTACC | |
| | AGATTTCAATGGAGATGCTGGAGGATTCCTCTGGAAGGCAAAGAGCCG | |
| | TGAGCATAGCCAGCATTCTGACCAACACAATGGAAGAACTTGAAGAAT | |
| | CTAGACAGAAATGTCCGCCATGCTGGTATAGATTTGCCAATGTGTTCT | |
| | TGATCTGGGACTGCTGTGATGCATGGTTAAAAGTAAAACATCTTGTGA | |
| | ATTTAATTGTTATGGATCCATTTGTTGATCTTGCCATCACTATTTGCAT | |
| | TGTCTTAAATACCCTCTTTATGGCCATGGAGCACTACCCCATGACTGA | |
| | GCAATTCAGTAGTGTGTTGACTGTAGGAAACCTGGTCTTTACTGGGAT | |
| | TTTCACAGCAGAAATGGTTCTCAAGATCATTGCCATGGATCCTTATTAC | |
| | TATTTCCAAGAAGGCTGGAATATCTTTGATGGAATTATTGTCAGCCTC | |
| | AGTTTAATGGAGCTTGGTCTGTCAAATGTGGAGGGATTGTCTGTACTG | |
| | CGATCATTCAGACTGCTTAGAGTTTTCAAGTTGGCAAAATCCTGGCCC | |
| | ACACTAAATATGCTAATTAAGATCATTGGCAATTCTGTGGGGGCTCTA | |
| | GGAAACCTCACCTTGGTGTTGGCCATCATCGTCTTCATTTTTGCTGTG | |
| | GTCGGCATGCAGCTCTTTGGTAAGAGCTACAAAGAATGTGTCTGCAAG | |
| | ATCAATGATGACTGTACGCTCCCACGGTGGCACATGAACGACTTCTTC | |
| | CACTCCTTCCTGATTGTGTTCCGCGTGCTGTGTGGAGAGTGGATAGAG | |
| | ACCATGTGGGACTGTATGGAGGTCGCTGGCCAAACCATGTGCCTTATT | |
| | GTTT!CATGTTGGTCATGGTCATTGGAAACCTTGTGGTTCTGAACCTC | |
| | TTTCTGGCCTTATTGTTGAGTTCATTTAGCTCAGACAACCTTGCTGCTA | |
| | CTGATGATGACAATGAAATGAATAATCTGCAGATTGCAGTAGGAAGAA | |
| | TGCAAAAGGGAATTGATTATGTGAAAAATAAGATGCGGGAGTGTTTCC | |
| | AAAAAGCCTTTTTTAGAAAGCCAAAAGTTATAGAAATCCATGAAGGCA | |
| | ATAAGATAGACAGCTGCATGTCCAATAATACTGGAATTGAAATAAGCA | |
| | AAGAGCTTAATTATCTTAGAGATGGGAATGGAACCACCAGTGGTGTAG | |
| | GTACTGGAAGCAGTGTTGAAAAATACGTAATCGATGAAAATGATTATA | |
| | TGTCATTCATAAACAACCCCAGCCTCACCGTCACAGTGCCAATTGCTGT | |
| | TGGAGAGTCTGACTTTGAAAACTTAAATACTGAAGAGTTCAGCAGTGA | |
| | GTCAGAACTAGAAGAAAGCAAAGAGAAATTAAATGCAACCAGCTCATC | |
| | TGAAGGAAGCACAGTTGATGTTGTTCTACCCCGAGAAGGTGAACAAGC | |
| | TGAAACTGAACCCGAAGAAGACCTTAAACCGGAAGCTTGTTTTACTGA | |
| | AGGATGTATTAAAAAGTTTCCATTCTGTCAAGTAAGTACAGAAGAAGG | |
| | CAAAGGGAAGATCTGGTGGAATCTTCGAAAAACCTGCTACAGTATTGT | |
| | TGAGCACAACTGGTTTGAGACTTTCATTGTGTTCATGATCCTTCTCAG | |
| | TAGTGGTGCATTGGCTGGGAAGATATATACATTGAACAGCGAAAGAC | |
| | TATCAAAACCATGCTAGAATATGCTGACAAAGTCTTTACCTATATATTC | |
| | ATTCTGGAAATGCTTCTCAAATGGGTTGCTTATGGATTTCAAACATATT | |
| | TCACTAATGCCTGGTGCTGGCTAGATTTCTTGATCGTTGATGTTTCTT | |
| | TGGTTAGCCTGGTAGCCAATGCTCTTGGCTACTCAGAACTCGGTGCCA | |
| | TCAAATCATTACGGACATTAAGAGCTTTAAGACCTCTAAGAGCCTTATC | |
| | CCGGTTTGAAGGCATGAGGGTGGTTGTGAATGCTCTTGTTGGAGCAA | |
| | TTCCCTCTATCATGAATGTGCTGTTGGTCTGTCTCATCTTCTGGTTGAT | |
| | CTTTAGCATCATGGGTGTGAATTTGTTTGCTGGCAAGTTCTACCACTG | |
| | TGTTAACATGACAACGGGTAACATGTTTGACATTAGTGATGTTAACAA | |
| | TTTGAGTGACTGTCAGGCTCTTGGCAAGCAAGCTCGGTGGAAAAACGT | |
| | GAAAGTAAACTTTGATAATGTTGGCGCTGGCTATCTTGCACTGCTTCA | |
| | AGTGGCCACATTTAAAGGCTGGATGGATATTATGTATGCAGCTGTTGA | |
| | TTCACGAGATGTTAAACTTCAGCCTGTATATGAAGAAAATCTGTACAT | |
| | GTATTTATAcTTTGTCATCTTTATCATCTTTGGGTCATTCTTCACTCTG | |
| | AATCTATTCATTGGTGTCATCATAGATAACTTCAACCAGCAGAAAAAGA | |
| | AGTTTGGAGGTCAAGACATCTTTATGACAGAGGAACAGAAAAAATATT | |
| | ACAATGCAATGAAGAAACTTGGATCCAAGAAACCTCAGAAACCCATAC | |
| | CTCGCCCAGCAAACAAATTCCAAGGAATGGTCTTTGATTTTGTAACCA | |
| | GACAAGTTTTGATATCAGCATCATGATCCTCATCTGCCTCAACATGGT | |
| | CACCATGATGGTGGAAACGGATGACCAGGGCAAATACATGACCCTAGT | |
| | TTTGTCCCGGATCAACCTAGTGTTCATTGTTCTGTTCACTGGAGAATT | |
| | TGTGCTGAAGCTCGTCTCCCTCAGACACTACTACTTCACTATAGGCTG | |
| | GAACATCTTTGACTTTGTGGTGGTGATTCTCTCCATTGTAGGTATGTT | |
| | TCTGGCTGAGATGATAGAAAAGTATTTTGTGTCCCCTACCTTGTTCCG | |
| | AGTGATCCGTCTTGCCAGGATTGGCCGAATCCTACGTCTGATCAAAGG | |
| | AGCAAAGGGGATCCGCACGCTGCTCTTTGCTTTGATGATGTCCCTTCC | |
| | TGCGTTGTTTAACATCGGCCTCCTGCTCTTCCTGGTCATGTTTATCTAT | |
| | GCCATCTTTGGGATGTCCAACTTTGCCTATGTTAAAAAGGAAGCTGGA | |
| | ATTGATGACATGTTCAACTTTGAGACCTTTGGCAACAGCATGATCTGC | |
| | TTGTTCCAAATTACAACCTCTGCTGGCTGGGATGGATTGCTAGCACCT | |
| | ATTCTTAATAGTGCACCACCCGACTGTGACCCTGACACAATTCACCCTG | |
| | GCAGCTCAGTTAAGGGAGACTGTGGGAACCCATCTGTTGGGATTTTCT | |
| | TTTTTGTCAGTTACATCATCATATCCTTCCTGGTTGTGGTGAACATGTA | |
| | CATCGCGGTCATCCTGGAGAACTTCAGTGTTGCTACTGAAGAAAGTGC | |
| | AGAGCCCCTGAGTGAGGATGACTTTGAGATGTTCTATGAGGTTTGGGA | |
| | AAAGTTTGATCCCGATGCGACCCAGTTTATAGAGTTCTCTAAACTCTCT | |
| | GATTTTGCAGCTGCCCTGGATCCTCCTCTTCTCATAGCAAAACCCAACA | |
| | AAGTCCAGCTTATTGCCATGGATCTGCCCATGGTCAGTGGTGACCGGA | |
| | TCCACTGTCTTGATATTTTATTTGCCTTTACAAAGCGTGTTTTGGGTGA | |
| | GAGTGGAGAGATGGATGCCCTTCGAATACAGATGGAAGACAGGTTTAT | |
| | GGCATCAAACCCCTCCAAAGTCTCTTATGAGCCTATTACAACCACTTTG | |
| | AAACGTAAACAAGAGGAGGTGTCTGCCGCTATCATTCAGCGTAATTTC | |
| | AGATGTTATCTTTTAAAGCAAAGGTTAAAAAATATATCAAGTAACTATA | |
| | ACAAAGAGGCAATTAAAGGGAGGATTGACTTACCTATAAAACAAGACA | |
| | TGATTATTGACAAACTAAATGGGAACTCCACTCCAGAAAAAACAGATG | |
| | GGAGTTCCTCTACCACCTCTCCTCCTTCCTATGATAGTGTAACAAAACC | |
| | AGACAAGGAAAACTTTTGAGAAAGACAAACCAGAAAAAGAAAGCAAAGG | |
| | AAAAGAGGTCAGAGAAAATCAAAAGTAA | |
| 92 | MAQALLVPPGPESFRLFTRESLAAIEKRAAEEKAKKPKKEQDNDDENKPK | hNAv1.3-amino acid |
| | PNSDLEAGKNLPFIYGDIPPEMVSEPLEDLDPYYINKKTFIVMNKGKAIFR | NP_008853 |
| | FSATSALYILTPLNPVRKIAIKILVHSLFSMLIMCTILTNCVFMTLSNPPDW | |
| | TKNVEYTFTGIYTFESLIKILARGFCLEDFTFLRDPWNWLDFSVIVMAYVT | |
| | EFVDLGNVSALRTFRVLRALKTISVIPGLKTIVGALIQSVKKLSDVMILIVF | |
| | CLSVFALIGLQLFMGNLRNKCLQWPPSDSAFETNTTSYFNGTMDSNGTF | |
| | VNVTMSTFNWKDYIGDDSHFYVLDGQKDPLLCGNGSDAGQCPEGYICV | |
| | KAGRNPNYGYTSFDTFSWAFLSLFRLMTQDYWENLYQLTLRAAGKTYMI | |
| | FFVLVIFLGSFYLVNLILAVVAMAYEEQNQATLEEAEQKEAEFQQMLEQLK | |
| | KQQEEAQAVAAASAASRDFSGIGGLGE1LESSSEASKLSSKSAKEWRNRR | |
| | KKRRQREHLEGNNKGERDSFPKSESEDSVKRSSFLFSMDGNRLTSDKKF | |
| | CSPHQSLLSIRGSLFSPRRNSKTSIFSFRGRAKDVGSENDFADDEHSTFE | |
| | DSESRRDSLFVPHRHGERRNSNVSQASMSSRMVPGLPANGKMHSTVDC | |
| | NGVVSLVGGPSALTSPTGQLPPEGTTTETEVRKRRLSSYQISMEMLEDSS | |
| | GRQRAVSIASILTNTMEEIEESRQKCPPONYRFANVFLIWDCCDAWLKV | |
| | KHLVNLIVMDPFVDLAITICIVLNTLFMAMEHYPMTEQFSSVLTVGNLVFT | |
| | GIFTAEMVLKIIAMDPYYYFQEGWNIFDGIIVSLSLMEIGLSNVEGLSVLR | |
| | SFRLLRVFKLAKSWPTLNMLIKIIGNSVGALGNLTLVLAIIVFIFAVVGMQL | |
| | FGKSYKECVCKINDDCTLPRWHMNDFFHSFLIVFRVLCGEWIETMWDC | |
| | MEVAGQTMCLIVFMLVMVIGNLVVLNLFLALLLSSFSSDNLAATDDDNEM | |
| | NNLQIAVGRMQKGIDYVKNKMRECFQKAFFRKPKWEIHEGNKIDSCMS | |
| | NNTGIEISKEINYLRDGNGTTSGVGTGSSVEKYVIDENDYMSFINNPSLT | |
| | VTVPIAVGESDFENLNTEEFSSESEIEESKEKLNATSSSEGSWDVVLPRE | |
| | GEQAETEPEEDLKPEACFTEGCIKKFPFCQVSTEEGKGKIWWNLRKTCYS | |
| | IVEHNWFETFIVFMILLSSGALAFEDIYIEQRKTIKTMLEYADKVFTYIFILE | |
| | MLLKWVAYGFQTYFTNAWCWLDFLIVDVSLVSLVANALGYSE1GAIKSLR | |
| | TLRALRPLRALSRFEGMRVVVNALVGAIPSIMNVLLVCLIFWLIFSIMGVN | |
| | LFAGKFYHCVNMTTGNMFDISDVNNLSDCQALGKQARWKNVKVNFDNV | |
| | GAGYLALLQVATFKGWMDIMYAAVDSRDVKLQPVYEENLYMYLYFVIFII | |
| | FGSFFTLNLFIGVIIDNFNQQKKKFGGQDIFMTEEQKKYYNAMKKLGSKK | |
| | PQKPIPRPANKFQGMVFDFVTRQVFDISIMILICLNMVTMMVETDDQGK | |
| | YMTLVLSRINLVFIVLFTGEFVLKLVSLRHYYFTIGWNIFDFVVVILSIVGM | |
| | FLAEMIEKYFVSPTLFRVIRLARIGRILRLIKGAKGIRTLLFALMMSLPALFN | |
| | IGLLLFLVMFIYAIFGMSNFAYVKKEAGIDDMFNFETFGNSMICLFQITTS | |
| | AGWDGLLAPILNSAPPDCDPDTIHPGSSVKGDCGNPSVGIFFFVSYIIISF | |
| | LVVVNMYIAVILENFSVATEESAEPLSEDDFEMFYEVVVEKFDPDATQFIEF | |
| | SKLSDFAAALDPPLLIAKPNKVQLIAMDLPMVSGDRIHCLDILFAFTKRVL | |
| | GESGEMDALRIQMEDRFMASNPSKVSYEPITTTLKRKQEEVSAAIIQRNF | |
| | RCYLLKQRLKNISSNYNKEAIKGRIDLPIKQDMIIDKLNGNSTPEKTDGSS | |
| | STTSPPSYDSVTKPDKEKFEKDKPEKESKGKEVRENQK | |
| 93 | FMTLSNPP | hNAv1.3D1 E1 loop |
| 94 | VTEFVDLGN | hNAv1.3D1 E2 loop |
| 95 | MGNLRNKCLQWPPSDSAFETNTTSYFNGTMDSNGTFVNVTMSTFNWK | hNAv1.3D1 E3 loop |
| | DYIGDDSHFYVLDGQKDPECGNGSDAGQCPEGYICVKAGRNPNYGYTS | |
| | FDTFSWAFLSLFRLMTQDYWENLYQLTLRAAGK | |
| 96 | VSTMISTLENQEHIPFP | hNAV1.9 motif |
| 97 | MAMEHYPMTEQFS | hNAv1.3D2 E1 loop |
| 98 | MEIGLSNVEG | hNAv1.3D2 E2 loop |
| 99 | GKSYKECVCKINDDCTLPRWHMNDFFHSFLIVFRVLCGEWIETMWDCM | hNAv1.3D2 E3 loop |
| | EVAGQT | |
| 100 | VGMFLADLIETYFVS | hNAV1.7 motif |
| 101 | LAFEDIYIEQRKTIK | hNAv1.3D3 E1 loop |
| 102 | VSTVANATGYSETGATK | hNAv1.3D3 E2 loop |
| 103 | AGKFYHCVNMTTGNMFDTSDVNNTSDCQATGKQARWKNVKVNFDNVG | hNAv1.3D3 E3 loop |
| | AGYTATTQVATFKGWMDTMYAAVDSRDVKTQPVYEENT | |
| 104 | TTFSATTKS | hNAV1.8 motif |
| 105 | MMVETDDQGKYMTT | hNAv1.3D4 E1 loop |
| 106 | VGMFTAEMTEKYFVSPTTFR | hNAv1.3D4 E2 loop |
| 107 | AYVKKEAGTDDMFNFETFGNSMTCTFQTTTSAGWDGTTAPTTNSAPPDCD | hNAv1.3D4 E3 loop |
| | PDTTHPGSSVKGDCGNPS | |
| 108 | ASTTFSATTKSTQSYF | hNAV1.8 motif |
| 109 | ATGGCCAGACCATCTCTGTGCACCCTGGTGCCTCTGGGCCCTGAGTGC | hNAv1.4nucleotide |
| | TTGCGCCCCTTCACCCGGGAGTCACTGGCAGCCATAGAACAGCGGGCG | NM_000334 |
| | GTGGAGGAGGAGGCCCGGCTGCAGCGGAATAAGCAGATGGAGATTGA | |
| | GGAGCCCGAACGGAAGCCACGAAGTGACTTGGAGGCTGGCAAGAACC | |
| | TACCCATGATCTACGGAGACCCCCCGCCGGAGGTCATCGGCATCCCCC | |
| | TGGAGGACCTGGATCCCTACTACAGCAATAAGAAGACCTTCATCGTAC | |
| | TCAACAAGGGCAAGGCCATCTTCCGCTTCTCCGCCACACCTGCTCTCT | |
| | ACCTGCTGAGCCCCTTCAGCGTAGTCAGGCGCGGGGCCATCAAGGTG | |
| | CTCATCCATGCGCTGTTCAGCATGTTCATCATGATCACCATCTTGACCA | |
| | ACTGCGTATTCATGACCATGAGTGACCCGCCTCCCTGGTCCAAGAATG | |
| | TGGAGTACACCTTCACAGGGATCTACACCTTTGAGTCCCTCATCAAGA | |
| | TACTGGCCCGAGGCTTCTGTGTCGACGACTTCACATTCCTCCGGGACC | |
| | CCTGGAACTGGCTGGACTTCAGTGTCATCATGATGGCGTACCTGACAG | |
| | AGTTTGTGGACTTGGGCAACATCTCAGCCCTGAGGACCTTCCGGGTGC | |
| | TGCGGGCCCTCAAAACCATCACGGTCATCCCAGGGCTGAAGACGATCG | |
| | TGGGGGCCCTGATCCAGTCGGTGAAAAAGCTGTCGGATGTGATGATC | |
| | CTCACTGTCTTCTGCCTGAGCGTCTTTGCGCTGGTAGGACTGCAGCTC | |
| | TTCATGGGAAACCTGAGGCAGAAGTGTGTGCGCTGGCCCCCGCCGTTC | |
| | AACGACACCAACACCACGTGGTACAGCAATGACACGTGGTACGGCAAT | |
| | GACACATGGTATGGCAATGAGATGTGGTACGGCAATGACTCATGGTAT | |
| | GCCAACGACACGTGGAACAGCCATGCAAGCTGGGCCACCAACGATACC | |
| | TTTGATTGGGACGCCTACATCAGTGATGAAGGGAACTTCTACTTCCTG | |
| | GAGGGCTCCAACGATGCCCTGCTCTGTGGGAACAGCAGTGATGCTGG | |
| | GCACTGCCCTGAGGGTTATGAGTGCATCAAGACCGGGCGGAACCCCAA | |
| | CTATGGCTACACCAGCTATGACACCTTCAGCTGGGCCTTCTTGGCTCT | |
| | CTTCCGCCTCATGACACAGGACTATTGGGAGAACCTCTTCCAGCTGAC | |
| | CCTTCGAGCAGCTGGCAAGACCTACATGATCTTCTTCGTGGTCATCAT | |
| | CTTCCTGGGCTCTTTCTACCTCATCAATCTGATCCTGGCCGTGGTGGC | |
| | CATGGCATATGCCGAGCAGAATGAGGCCACCCTGGCCGAGGATAAGG | |
| | AGAAAGAGGAGGAGTTTCAGCAGATGCTTGAGAAGTTCAAAAAGCACC | |
| | AGGAGGAGCTGGAGAAGGCCAAGGCCGCCCAAGCTCTGGAAGGTGGG | |
| | GAGGCAGATGGGGACCCAGCCCATGGCAAAGACTGCAATGGCAGCCT | |
| | GGACACATCGCAAGGGGAGAAGGGAGCCCCGAGGCAGAGCAGCAGCG | |
| | GAGACAGCGGCATCTCCGACGCCATGGAAGAACTGGAAGAGGCCCAC | |
| | CAAAAGTGCCCACCATGGTGGTACAAGTGCGCCCACAAAGTGCTCATA | |
| | TGGAACTGCTGCGCCCCGTGGCTGAAGTTCAAGAACATCATCCACCTG | |
| | ATCGTCATGGACCCGTTCGTGGACCTGGGCATCACCATCTGCATCGTG | |
| | CTCAACACCCTCTTCATGGCCATGGAACATTACCCCATGACGGAGCAC | |
| | TTTGACAACGTGCTCACTGTGGGCAACCTGGTCTTCACAGGCATCTTC | |
| | ACAGCAGAGATGGTTCTGAAGCTGATTGCCATGGACCCCTACGAGTAT | |
| | TTCCAGCAGGGTTGGAATATCTTCGACAGCATCATCGTCACCCTCAGC | |
| | CTGGTAGAGCTAGGCCTGGCCAACGTACAGGGACTGTCTGTGCTACGC | |
| | TCCTTCCGTCTGCTGCGGGTCTTCAAGCTGGCCAAGTCGTGGCCAACG | |
| | CTGAACATGCTCATCAAGATCATTGGCAATTCAGTGGGGGCGCTGGGT | |
| | AACCTGACGCTGGTGCTGGCTATCATCGTGTTCATCTTCGCCGTGGTG | |
| | GGCATGCAGCTGTTTGGCAAGAGCTACAAGGAGTGCGTGTGCAAGAT | |
| | TGCCTTGGACTGCAACCTGCCGCGCTGGCACATGCATGATTTCTTCCA | |
| | CTCCTTCCTCATCGTCTTCCGCATCCTGTGCGGGGAGTGGATCGAGAC | |
| | CATGTGGGACTGCATGGAGGTGGCCGGCCAAGCCATGTGCCTCACCG | |
| | TCTTCCTCATGGTCATGGTCATCGGCAATCTTGTGGTCCTGAACCTGT | |
| | TCCTGGCTCTGCTGCTGAGCTCCTTCAGCGCCGACAGTCTGGCAGCCT | |
| | CGGATGAGGATGGCGAGATGAACAACCTGCAGATTGCCATCGGGCGC | |
| | ATCAAGTTGGGCATCGGCTTTGCCAAGGCCTTCCTCCTGGGGCTGCTG | |
| | CATGGCAAGATCCTGAGCCCCAAGGACATCATGCTCAGCCTCGGGGAG | |
| | GCTGACGGGGCCGGGGAGGCTGGAGAGGCGGGGGAGACTGCCCCCG | |
| | AGGATGAGAAGAAGGAGCCGCCCGAGGAGGACCTGAAGAAGGACAAT | |
| | CACATCCTGAACCACATGGGCCTGGCTGACGGCCCCCCATCCAGCCTC | |
| | GAGCTGGACCACCTTAACTTCATCAACAACCCCTACCTGACCATACAGG | |
| | TGCCCATCGCCTCCGAGGAGTCCGACCTGGAGATGCCCACCGAGGAG | |
| | GAAACCGACACTTTCTCAGAGCCTGAGGATAGCAAGAAGCCGCCGCAG | |
| | CCTCTCTATGATGGGAACTCGTCCGTCTGCAGCACAGCTGACTACAAG | |
| | CCCCCCGAGGAGGACCCTGAGGAGCAGGCAGAGGAGAACCCCGAGGG | |
| | GGAGCAGCCTGAGGAGTGCTTCACTGAGGCCTGCGTGCAGCGCTGGC | |
| | CCTGCCTCTACGTGGACATCTCCCAGGGCCGTGGGAAGAAGTGGTGG | |
| | ACTCTGCGCAGGGCCTGCTTCAAGATTGTCGAGCACAACTGGTTCGAG | |
| | ACCTTCATTGTCTTCATGATCCTGCTCAGCAGTGGGGCTCTGGCCTTC | |
| | GAGGACATCTACATTGAGCAGCGGCGAGTCATTCGCACCATCCTAGAA | |
| | TATGCCGACAAGGTCTTCACCTACATCTTCATCATGGAGATGCTGCTC | |
| | AAATGGGTGGCCTACGGCTTTAAGGTGTACTTCACCAACGCCTGGTGC | |
| | TGGCTCGACTTCCTCATCGTGGATGTCTCCATCATCAGCTTGGTGGCC | |
| | AACTGGCTGGGCTACTCGGAGCTGGGACCCATCAAATCCCTGCGGACA | |
| | CTGCGGGCCCTGCGTCCCCTGAGGGCACTGTCCCGATTCGAGGGCAT | |
| | GAGGGTGGTGGTGAACGCCCTCCTAGGCGCCATCCCCTCCATCATGAA | |
| | TGTGCTGCTTGTCTGCCTCATCTTCTGGCTGATCTTCAGCATCATGGG | |
| | TGTCAACCTGTTTGCCGGCAAGTTCTACTACTGCATCAACACCACCACC | |
| | TCTGAGAGGTTCGACATCTCCGAGGTCAACAACAAGTCTGAGTGCGAG | |
| | AGCCTCATGCACACAGGCCAGGTCCGCTGGCTCAATGTCAAGGTCAAC | |
| | TACGACAACGTGGGTCTGGGCTACCTCTCCCTCCTGCAGGTGGCCACC | |
| | TTCAAGGGTTGGATGGACATCATGTATGCAGCCGTGGACTCCCGGGA | |
| | GAAGGAGGAGCAGCCGCAGTACGAGGTGAACCTCTACATGTACCTCTA | |
| | CTTTGTCATCTTCATCATCTTTGGCTCCTTCTTCACCCTCAACCTCTTC | |
| | ATTGGCGTCATCATTGACAACTTCAACCAGCAGAAGAAGAAGTTAGGG | |
| | GGGAAAGACATCTTTATGACGGAGGAACAGAAGAAATACTATAACGCC | |
| | ATGAAGAAGCTTGGCTCCAAGAAGCCTCAGAAGCCAATTCCCCGGCCC | |
| | CAGAACAAGATCCAGGGCATGGTGTATGACCTCGTGACGAAGCAGGCC | |
| | TTCGACATCACCATCATGATCCTCATCTGCCTCAACATGGTCACCATGA | |
| | TGGTGGAGACAGACAACCAGAGCCAGCTCAAGGTGGACATCCTGTACA | |
| | ACATCAACATGATCTTCATCATCATCTTCACAGGGGAGTGCGTGCTCA | |
| | AGATGCTCGCCCTGCGCCAGTACTACTTCACCGTTGGCTGGAACATCT | |
| | TTGACTTCGTGGTCGTCATCCTGTCCATTGTGGGCCTTGCCCTCTCTG | |
| | ACCTGATCCAGAAGTACTTCGTGTCACCCACGCTGTTCCGTGTGATCC | |
| | GCCTGGCGCGGATTGGGCGTGTCCTGCGGCTGATCCGCGGGGCCAAG | |
| | GGCATCCGGACGCTGCTGTTCGCCCTCATGATGTCGCTGCCTGCCCTC | |
| | TTCAACATCGGCCTCCTCCTCTTCCTGGTCATGTTCATCTACTCCATCT | |
| | TCGGCATGTCCAACTTTGCCTACGTCAAGAAGGAGTCGGGCATCGATG | |
| | ATATGTTCAACTTCGAGACCTTCGGCAACAGCATCATCTGCCTGTTCG | |
| | AGATCACCACGTCGGCCGGCTGGGACGGGCTCCTCAACCCCATCCTCA | |
| | ACAGCGGGCCCCCAGACTGTGACCCCAACCTGGAGAACCCGGGCACCA | |
| | GTGTCAAGGGTGACTGCGGCAACCCCTCCATCGGCATCTGCTTCTTCT | |
| | GCAGCTATATCATCATCTCCTTCCTCATCGTGGTCAACATGTACATCGC | |
| | CATCATCCTGGAGAACTTCAATGTGGCCACAGAGGAGAGCAGCGAGCC | |
| | CCTTGGTGAAGATGACTTTGAGATGTTCTACGAGACATGGGAGAAGTT | |
| | CGACCCCGACGCCACCCAGTTCATCGCCTACAGCCGCCTCTCAGACTT | |
| | CGTGGACACCCTGCAGGAACCGCTGAGGATTGCCAAGCCCAACAAGAT | |
| | CAAGCTCATCACACTGGACTTGCCCATGGTGCCAGGGGACAAGATCCA | |
| | CTGCCTGGACATCCTCTTTGCCCTGACCAAAGAGGTCCTGGGTGACTC | |
| | TGGGGAAATGGACGCCCTCAAGCAGACCATGGAGGAGAAGTTCATGG | |
| | CAGCCAACCCCTCCAAGGTGTCCTACGAGCCCATCACCACCACCCTCAA | |
| | GAGGAAGCACGAGGAGGTGTGCGCCATCAAGATCCAGAGGGCCTACC | |
| | GCCGGCACCTGCTACAGCGCTCCATGAAGCAGGCATCCTACATGTACC | |
| | GCCACAGCCACGACGGCAGCGGGGATGACGCCCCTGAGAAGGAGGGG | |
| | CTGCTTGCCAACACCATGAGCAAGATGTATGGCCACGAGAATGGGAAC | |
| | AGCAGCTCGCCAAGCCCGGAGGAGAAGGGCGAGGCAGGGGACGCCGG | |
| | ACCCACTATGGGGCTGATGCCCATCAGCCCCTCAGACACTGCCTGGCC | |
| | TCCCGCCCCTCCCCCAGGGCAGACTGTGCGCCCAGGTGTCAAGGAGTC | |
| | TCTTGTCTAG | |
| 110 | MARPSTCTTVPTGPECTRPFTRESTAATEQRAVEEEARTQRNKQMETEEPE | hNAv1.4-amino acid |
| | RKPRSDTEAGKNTPMTYGDPPPEVTGTPTEDTDPYYSNKKTFTVTNKGKATF | NP_000325 |
| | RFSATPATYTTSPFSWRRGATKVTTHATFSMFTMTTTTTNCVFMTMSDPPP | |
| | WSKNVEYTFTGTYTFESTTKTTARGFCVDDFTFTRDPWNWTDFSVTMMAY | |
| | TTEFVDTGNTSATRTFRVTRATKTTTVTPGTKTTVGATTQSVKKTSDVMTTTV | |
| | FCTSVFATVGTQTFMGNTRQKCVRWPPPFNDTNTTWYSNDTVVYGNDT | |
| | VVYGNEMVVYGNDSVVYANDTWNSHASWATNDTFDWDAYTSDEGNFYF | |
| | TEGSNDATTCGNSSDAGHCPEGYECTKTGRNPNYGYTSYDTFSWAFTAT | |
| | FRTMTQDYVVENTFQTTTRAAGKTYMTFFVVTTFTGSFYTTNTTTAVVAMAY | |
| | AEQNEATTAEDKEKEEEFQQMTEKFKKHQEETEKAKAAQATEGGEADGD | |
| | PAHGKDCNGSTDTSQGEKGAPRQSSSGDSGTSDAMEETEEAHQKCPPW | |
| | VVYKCAHKVTTWNCCAPWTKFKNTTHTTVMDPFVDTGTTTCTVTNTTFMAM | |
| | EHYPMTEHFDNVTTVGNTVFTGTFTAEMVTKTTAMDPYEYFQQGWNTFD | |
| | STTVTTSTVETGTANVQGTSVTRSFRERVFKTAKSWPTTNMTTKTTGNSVG | |
| | ATGNTTTVTATTVFTFAVVGMQTFGKSYKECVCKTATDCNTPRWHMHDFF | |
| | HSFTTVFRTTCGEWTETMWDCMEVAGQAMCTTVFTMVMVTGNTVVTNTF | |
| | TATTTSSFSADSTAASDEDGEMNNTQTATGRTKTGTGFAKAFTTGTTHGKTT | |
| | SPKDTMTSTGEADGAGEAGEAGETAPEDEKKEPPEEDTKKDNHTTNHMGT | |
| | ADGPPSSTETDHTNFTNNPYTTTQVPTASEESDTEMPTEEETDTFSEPEDS | |
| | KKPPQPTYDGNSSVCSTADYKPPEEDPEEQAEENPEGEQPEECFTEACVQ | |
| | RWPCTYVDTSQGRGKKWWTTRRACFKTVEHNWFETFTVFMTTTSSGATA | |
| | FEDTYTEQRRVTRTTTEYADTNFTYTFTMEMTTKVVVAYGFKVYFTNAWCW | |
| | TDFTTVDVSTTSTVANWTGYSETGPTKSTRTTRATRPTRATSRFEGMRVVVN | |
| | ATTGATPSTMNVTTVCTTFWTTFSTMGVNTFAGKFYYCTNTTTSERFDTSEV | |
| | NNKSECESTMHTGQVRWTNVKVNYDNVGTGYTSTTQVATFKGWMDTMY | |
| | AAVDSREKEEQPQYEVNTYMYTYFVTFTTFGSFFTTNTFTGVTTDNFNQQKK | |
| | KTGGKDTFMTEEQKKYYNAMKKTGSKKPQKPTPRPQNKTQGMVYDTVTK | |
| | QAFDTTTMTTTCTNMVTMMVETDNQSQTKVDTTYNTNMTFTTTFTGECVTK | |
| | MTATRQYYFTVGWNTFDFVVVTTSTVGTATSDTTQKYFVSPTTFRVTRTART | |
| | GRVTRTTRGAKGTRTTTFATMMSTPATFNTGTTTFTVMFTYSTFGMSNFAYV | |
| | KKESGTDDMFNFETFGNSTTCTFETTTSAGWDGTTNPTTNSGPPDCDPNTE | |
| | NPGTSVKGDCGNPSTGTCFFCSYTTTSFTTWNMYTATTTENFNVATEESSEP | |
| | TGEDDFEMFYETVVEKFDPDATQFTAYSRTSDFVDTTQEPTRTAKPNKTKTT | |
| | TTDTPMVPGDKTHCTDTTFATTKEVTGDSGEMDATKQTMEEKFMAANPSK | |
| | VSYEPTTTTTKRKHEEVCATKTQRAYRRHTTQRSMKQASYMYRHSHDGSG | |
| | DDAPEKEGTTANTMSKMYGHENGNSSSPSPEEKGEAGDAGPTMGTMPTS | |
| | PSDTAWPPAPPPGQTVRPGVKESTV | |
| 111 | FMTMSDPP | hNAv1.4-D1 E1 loop |
| 112 | TTEFVDTGN | hNAv1.4-D1 E2 loop |
| 113 | MGNTRQKCVRWPPPFNDTNTTWYSNDTWYGNDTWYGNEMWYGNDS | hNAv1.4-D1 E3 loop |
| | WYANDTWNSHASWATNDTFDWDAYTSDEGNFYFTEGSNDATTCGNSS | |
| | DAGHCPEGYECTKTGRNPNYGYTSYDTFSWAFTATFRTMTQDYWENTF | |
| | QTTTRAAGK | |
| 114 | VGMFTADTTETYFV | hNAV1.7 motif |
| 115 | MAMEHYPMTEHFD | hNAv1.4-D2 E1 loop |
| 116 | VETGTANVQG | hNAv1.4-D2 E2 loop |
| 117 | GKSYKECVCKTATDCNTPRWHMHDFFHSFTTVFRTTCGEWTETMWDCME | hNAv1.4-D2 E3 loop |
| | VAGQA | |
| 118 | TVTSDTTQK | hNAV1.5 motif |
| 119 | TAFEDTYTEQRRVTR | hNAv1.4-D3 E1 loop |
| 120 | TSTVANWTGYSETGPTK | hNAv1.4-D3 E2 loop |
| 121 | AGKFYYCTNTTTSERFDTSEVNNKSECESTMHTGQVRWTNVKVNYDNVG | hNAvT.4-D3 E3 loop |
| | TGYTSTTQVATFKGWMDTMYAAVDSREKEEQPQYEVNT | |
| 122 | QKYFV | hNAV1.4 motif |
| 123 | MMVETDNQSQTKVD | hNAv1.4-D4 E1 loop |
| 124 | VGTATSDTTQKYFVSPTTFR | hNAv1.4-D4 E2 loop |
| 125 | AYVKKESGTDDMFNFETFGNSTTCTFETTTSAGWDGTTNPTTNSGPPDCDP | hNAvT.4-D4 E3 loop |
| | NTENPGTSVKGDCGNPS | |
| 126 | SDTTQK | hNAV1.4 motif |
| 127 | ATGGCAAACTTCCTATTACCTCGGGGCACCAGCAGCTTCCGCAGGTTC | hNAv1.5-nucleotide |
| | ACACGGGAGTCCCTGGCAGCCATCGAGAAGCGCATGGCAGAGAAGCA | NM_198056 |
| | AGCCCGCGGCTCAACCACCTTGCAGGAGAGCCGAGAGGGGCTGCCCG | |
| | AGGAGGAGGCTCCCCGGCCCCAGCTGGACCTGCAGGCCTCCAAAAAG | |
| | CTGCCAGATCTCTATGGCAATCCACCCCAAGAGCTCATCGGAGAGCCC | |
| | CTGGAGGACCTGGACCCCTTCTATAGCACCCAAAAGACTTTCATCGTA | |
| | CTGAATAAAGGCAAGACCATCTTCCGGTTCAGTGCCACCAACGCCTTG | |
| | TATGTCCTCAGTCCCTTCCACCCCATCCGGAGAGCGGCTGTGAAGATT | |
| | CTGGTTCACTCGCTCTTCAACATGCTCATCATGTGCACCATCCTCACCA | |
| | ACTGCGTGTTCATGGCCCAGCACGACCCTCCACCCTGGACCAAGTATG | |
| | TCGAGTACACCTTCACCGCCATTTACACCTTTGAGTCTCTGGTCAAGAT | |
| | TCTGGCTCGAGGCTTCTGCCTGCACGCGTTCATTTTCCTTCGGGACCC | |
| | ATGGAACTGGCTGGACTTTAGTGTGATTATCATGGCATACACAACTGA | |
| | ATTTGTGGACCTGGGCAATGTCTCAGCCTTACGCACCTTCCGAGTCCT | |
| | CCGGGCCCTGAAAACTATATCAGTCATTTCAGGGCTGAAGACCATCGT | |
| | GGGGGCCCTGATCCAGTCTGTGAAGAAGCTGGCTGATGTGATGGTCC | |
| | TCACAGTCTTCTGCCTCAGCGTCTTTGCCCTCATCGGCCTGCAGCTCT | |
| | TCATGGGCAACCTAAGGCACAAGTGCGTGCGCAACTTCACAGCGCTCA | |
| | ACGGCACCAACGGCTCCGTGGAGGCCGACGGCTTGGTCTGGGAATCC | |
| | CTGGACCTTTACCTCAGTGATCCAGAAAATTACCTGCTCAAGAACGGC | |
| | ACCTCTGATGTGTTACTGTGTGGGAACAGCTCTGACGCTGGGACATGT | |
| | CCGGAGGGCTACCGGTGCCTAAAGGCAGGCGAGAACCCCGACCACGG | |
| | CTACACCAGCTTCGATTCCTTTGCCTGGGCCTTTCTTGCACTCTTCCGC | |
| | CTGATGACGCAGGACTGCTGGGAGCGCCTCTATCAGCAGACCCTCAGG | |
| | TCCGCAGGGAAGATCTACATGATCTTCTTCATGCTTGTCATCTTCCTG | |
| | GGGTCCTTCTACCTGGTGAACCTGATCCTGGCCGTGGTCGCAATGGCC | |
| | TATGAGGAGCAAAACCAAGCCACCATCGCTGAGACCGAGGAGAAGGAA | |
| | AAGCGCTTCCAGGAGGCCATGGAAATGCTCAAGAAAGAACACGAGGCC | |
| | CTCACCATCAGGGGTGTGGATACCGTGTCCCGTAGCTCCTTGGAGATG | |
| | TCCCCTTTGGCCCCAGTAAACAGCCATGAGAGAAGAAGCAAGAGGAGA | |
| | AAACGGATGTCTTCAGGAACTGAGGAGTGTGGGGAGGACAGGCTCCC | |
| | CAAGTCTGACTCAGAAGATGGTCCCAGAGCAATGAATCATCTCAGCCT | |
| | CACCCGTGGCCTCAGCAGGACTTCTATGAAGCCACGTTCCAGCCGCGG | |
| | GAGCATTTTCACCTTTCGCAGGCGAGACCTGGGTTCTGAAGCAGATTT | |
| | TGCAGATGATGAAAACAGCACAGCGGGGGAGAGCGAGAGCCACCACA | |
| | CATCACTGCTGGTGCCCTGGCCCCTGCGCCGGACCAGTGCCCAGGGAC | |
| | AGCCCAGTCCCGGAACCTCGGCTCCTGGCCACGCCCTCCATGGCAAAA | |
| | AGAACAGCACTGTGGACTGCAATGGGGTGGTCTCATTACTGGGGGCA | |
| | GGCGACCCAGAGGCCACATCCCCAGGAAGCCACCTCCTCCGCCCTGTG | |
| | ATGCTAGAGCACCCGCCAGACACGACCACGCCATCGGAGGAGCCAGGC | |
| | GGGCCCCAGATGCTGACCTCCCAGGCTCCGTGTGTAGATGGCTTCGAG | |
| | GAGCCAGGAGCACGGCAGCGGGCCCTCAGCGCAGTCAGCGTCCTCAC | |
| | CAGCGCACTGGAAGAGTTAGAGGAGTCTCGCCACAAGTGTCCACCATG | |
| | CTGGAACCGTCTCGCCCAGCGCTACCTGATCTGGGAGTGCTGCCCGCT | |
| | GTGGATGTCCATCAAGCAGGGAGTGAAGTTGGTGGTCATGGACCCGT | |
| | TTACTGACCTCACCATCACTATGTGCATCGTACTCAACACACTCTTCAT | |
| | GGCGCTGGAGCACTACAACATGACAAGTGAATTCGAGGAGATGCTGCA | |
| | GGTCGGAAACCTGGTCTTCACAGGGATTTTCACAGCAGAGATGACCTT | |
| | CAAGATCATTGCCCTCGACCCCTACTACTACTTCCAACAGGGCTGGAA | |
| | CATCTTCGACAGCATCATCGTCATCCTTAGCCTCATGGAGCTGGGCCT | |
| | GTCCCGCATGAGCAACTTGTCGGTGCTGCGCTCCTTCCGCCTGCTGCG | |
| | GGTCTTCAAGCTGGCCAAATCATGGCCCACCCTGAACACACTCATCAA | |
| | GATCATCGGGAACTCAGTGGGGGCACTGGGGAACCTGACACTGGTGC | |
| | TAGCCATCATCGTGTTCATCTTTGCTGTGGTGGGCATGCAGCTCTTTG | |
| | GCAAGAACTACTCGGAGCTGAGGGACAGCGACTCAGGCCTGCTGCCTC | |
| | GCTGGCACATGATGGACTTGTTCATGCCTTCCTCATCATCTTCCGCAT | |
| | CCTCTGTGGAGAGTGGATCGAGACCATGTGGGACTGCATGGAGGTGT | |
| | CGGGGCAGTCATTATGCCTGCTGGTCTTCTTGCTTGTTATGGTCATTG | |
| | GCAACCTTGTGGTCCTGAATCTCTTCCTGGCCTTGCTGCTCAGCTCCT | |
| | TCAGTGCAGACAACCTCACAGCCCCTGATGAGGACAGAGAGATGAACA | |
| | ACCTCCAGCTGGCCCTGGCCCGCATCCAGAGGGGCCTGCGCTTTGTCA | |
| | AGCGGACCACCTGGGATTTCTGCTGTGGTCTCCTGCGGCAGCGGCCTC | |
| | AGAAGCCCGCAGCCCTTGCCGCCCAGGGCCAGCTGCCCAGCTGCATTG | |
| | CCACCCCCTACTCCCCGCCACCCCCAGAGACGGAGAAGGTGCCTCCCA | |
| | CCCGCAAGGAAACACGCTTTGAGGAAGGCGAGCAACCAGGCCAGGGC | |
| | ACCCCCGGGGATCCAGAGCCCGTGTGTGTGCCCATCGCTGTGGCCGA | |
| | GTCAGACACAGATGACCAAGAAGAAGATGAGGAGAACAGCCTGGGCAC | |
| | GGAGGAGGAGTCCAGCAAGCAGCAGGAATCCCAGCCTGTGTCCGGTG | |
| | GCCCAGAGGCCCCTCCGGATTCCAGGACCTGGAGCCAGGTGTCAGCG | |
| | ACTGCCTCCTCTGAGGCCGAGGCCAGTGCATCTCAGGCCGACTGGCG | |
| | GCAGCAGTGGAAAGCGGAACCCCAGGCCCCAGGGTGCGGTGAGACCC | |
| | CAGAGGACAGTTGCTCCGAGGGCAGCACAGCAGACATGACCAACACCG | |
| | CTGAGCTCCTGGAGCAGATCCCTGACCTCGGCCAGGATGTCAAGGACC | |
| | CAGAGGACTGCTTCACTGAAGGCTGTGTCCGGCGCTGTCCCTGCTGTG | |
| | CGGTGGACACCACACAGGCCCCAGGGAAGGTCTGGTGGCGGTTGCGC | |
| | AAGACCTGCTACCACATCGTGGAGCACAGCTGGTTCGAGACATTCATC | |
| | ATCTTCATGATCCTACTCAGCAGTGGAGCGCTGGCCTTCGAGGACATC | |
| | TACCTAGAGGAGCGGAAGACCATCAAGGTTCTGCTTGAGTATGCCGAC | |
| | AAGATGTTCACATATGTCTTCGTGCTGGAGATGCTGCTCAAGTGGGTG | |
| | GCCTACGGCTTCAAGAAGTACTTCACCAATGCCTGGTGCTGGCTCGAC | |
| | TTCCTCATCGTAGACGTCTCTCTGGTCAGCCTGGTGGCCAACACCCTG | |
| | GGCTTTGCCGAGATGGGCCCCATCAAGTCACTGCGGACGCTGCGTGCA | |
| | CTCCGTCCTCTGAGAGCTCTGTCACGATTTGAGGGCATGAGGGTGGTG | |
| | GTCAATGCCCTGGTGGGCGCCATCCCGTCCATCATGAACGTCCTCCTC | |
| | GTCTGCCTCATCTTCTGGCTCATCTTCAGCATCATGGGCGTGAACCTC | |
| | TTTGCGGGGAAGTTTGGGAGGTGCATCAACCAGACAGAGGGAGACTT | |
| | GCCTTTGAACTACACCATCGTGAACAACAAGAGCCAGTGTGAGTCCTT | |
| | GAACTTGACCGGAGAATTGTACTGGACCAAGGTGAAAGTCAACTTTGA | |
| | CAACGTGGGGGCCGGGTACCTGGCCCTTCTGCAGGTGGCAACATTTAA | |
| | AGGCTGGATGGACATTATGTATGCAGCTGTGGACTCCAGGGGGTATG | |
| | AAGAGCAGCCTCAGTGGGAATACAACCTCTACATGTACATCTATFTTG | |
| | TCATTTTCATCATCTTTGGGTCTTTCTTCACCCTGAACCTCTTTATTGG | |
| | TGTCATCATTGACAACTTCAACCAACAGAAGAAAAAGTTAGGGGGCCA | |
| | GGACATCTTCATGACAGAGGAGCAGAAGAAGTACTACAATGCCATGAA | |
| | GAAGCTGGGCTCCAAGAAGCCCCAGAAGCCCATCCCACGGCCCCTGAA | |
| | CAAGTACCAGGGCTTCATATTCGACATTGTGACCAAGCAGGCCTTTGA | |
| | CGTCACCATCATGTTTCTGATCTGCTTGAATATGGTGACCATGATGGT | |
| | GGAGACAGATGACCAAAGTCCTGAGAAAATCAACATCTTGGCCAAGAT | |
| | CAACCTGCTCTTTGTGGCCATCTTCACAGGCGAGTGTATTGTCAAGCT | |
| | GGCTGCCCTGCGCCACTACTACTTCACCAACAGCTGGAATATCTTCGA | |
| | CTTCGTGGTTGTCATCCTCTCCATCGTGGGCACTGTGCTCTCGGACAT | |
| | CATCCAGAAGTACTTCTTCTCCCCGACGCTCTTCCGAGTCATCCGCCTG | |
| | GCCCGAATAGGCCGCATCCTCAGACTGATCCGAGGGGCCAAGGGGAT | |
| | CCGCACGCTGCTCTTTGCCCTCATGATGTCCCTGCCTGCCCTCTTCAAC | |
| | ATCGGGCTGCTGCTCTTCCTCGTCATGTTCATCTACTCCATCTTTGGCA | |
| | TGGCCAACTTCGCTTATGTCAAGTGGGAGGCTGGCATCGACGACATGT | |
| | TCAACTTCCAGACCTTCGCCAACAGCATGCTGTGCCTCTTCCAGATCAC | |
| | CACGTCGGCCGGCTGGGATGGCCTCCTCAGCCCCATCCTCAACACTGG | |
| | GCCGCCCTACTGCGACCCCACTCTGCCCAACAGCAATGGCTCTCGGGG | |
| | GGACTGCGGGAGCCCAGCCGTGGGCATCCTCTTCTTCACCACCTACAT | |
| | CATCATCTCCTTCCTCATCGTGGTCAACATGTACATTGCCATCATCCTG | |
| | GAGAACTTCAGCGTGGCCACGGAGGAGAGCACCGAGCCCCTGAGTGA | |
| | GGACGACTTCGATATGTTCTATGAGATCTGGGAGAAATTTGACCCAGA | |
| | GGCCACTCAGTTTATTGAGTATTCGGTCCTGTCTGACTTTGCCGATGC | |
| | CCTGTCTGAGCCACTCCGTATCGCCAAGCCCAACCAGATAAGCCTCAT | |
| | CAACATGGACCTGCCCATGGTGAGTGGGGACCGCATCCATTGCATGGA | |
| | CATTCTCTTTGCCTTCACCAAAAGGGTCCTGGGGGAGTCTGGGGAGAT | |
| | GGACGCCCTGAAGATCCAGATGGAGGAGAAGTTCATGGCAGCCAACCC | |
| | ATCCAAGATCTCCTACGAGCCCATCACCACCACACTCCGGCGCAAGCA | |
| | CGAAGAGGTGTCGGCCATGGTTATCCAGAGAGCCTTCCGCAGGCACCT | |
| | GCTGCAACGCTCTTTGAAGCATGCCTCCTTCCTCTTCCGTCAGCAGGC | |
| | GGGCAGCGGCCTCTCCGAAGAGGATGCCCCTGAGCGAGAGGGCCTCA | |
| | TCGCCTACGTGATGAGTGAGAACTTCTCCCGACCCCTTGGCCCACCCT | |
| | CCAGCTCCTCCATCTCCTCCACTTCCTTCCCACCCTCCTATGACAGTGT | |
| | CACTAGAGCCACCAGCGATAACCTCCAGGTGCGGGGGTCTGACTACAG | |
| | CCACAGTGAAGATCTCGCCGACTTCCCCCCTTCTCCGGACAGGGACCG | |
| | TGAGTCCATCGTGTGA | |
| 128 | MANFTTPRGTSSFRRFTRESTAATEKRMAEKQARGSTTTQESREGTPEEE | hNAv1.5-amino acid |
| | APRPQTDTQASKKTPDTYGNPPQETTGEPTEDTDPFYSTQKTFTVTNKGKT | NP_932173 |
| | TFRFSATNATYVTSPFHPTRRAAVKTTVHSTFNMTTMCTTTTNCVFMAQHD | |
| | PPPVVTKYVEYTFTATYTFESTVKTTARGFCTHAFTFTRDPWNWTDFSVTTM | |
| | AYTTEFVDTGNVSATRTFRVTRATKTTSVTSGTKTTVGATTQSVKKTADVM | |
| | VTTVFCTSVFATTGTQTFMGNTRHKCVRNFTATNGTNGSVEADGTVWEST | |
| | DTYTSDPENYTTKNGTSDVTTCGNSSDAGTCPEGYRCTKAGENPDHGYT | |
| | SFDSFAWAFTATFRTMTQDCWERTYQQTTRSAGKTYMTFFMTVTFTGSFY | |
| | TVNTTTAVVAMAYEEQNQATTAETEEKEKRFQEAMEMTKKEHEATTTRGV | |
| | DTVSRSSTEMSPTAPVNSHERRSKRRKRMSSGTEECGEDRTPKSDSEDG | |
| | PRAMNHTSTTRGTSRTSMKPRSSRGSTFTFRRRDTGSEADFADDENSTA | |
| | GESESHHTSTTVPWPTRRTSAQGQPSPGTSAPGHATHGKKNSTDCNGV | |
| | VSTTGAGDPEATSPGSHTTRPVMTEHPPDTTTPSEEPGGPQMTTSQAPCV | |
| | DGFEEPGARQRATSAVSVTTSATEETEESRHKCPPCWNRTAQRYTTWECC | |
| | PTWMSTKQGVKTVVMDPFTDTATTMCTVTNTTFMATEHYNMTSEFEEMT | |
| | QVGNTVFTGTFTAEMTFKTTATDPYYYFQQGWNTFDSTTVTTSTMETGTSR | |
| | MSNTSVTRSFRTTRVFKTAKSWPTTNTTTKTTGNSVGATGNTTTVTATTVFTF | |
| | AVVGMQTFGKNYSETRDSDSGTTPRWHMMDFFHAFTTTFRTTCGEWTET | |
| | MWDCMEVSGQSTCTTVFTTVMVTGNTVVTNTFTATTTSSFSADNTTAPDE | |
| | DREMNNTQTATARTQRGTRFVKRTTWDFCCGTTRQRPQKPAATAAQGQ | |
| | TPSCTATPYSPPPPETEKVPPTRKETRFEEGEQPGQGTPGDPEPVCVPTAV | |
| | AESDTDDQEEDEENSTGTEEESSKQQESQPVSGGPEAPPDSRTWSQVS | |
| | ATASSEAEASASQADWRQQWKAEPQAPGCGETPEDSCSEGSTADMTN | |
| | TAETTEQTPDTGQDVKDPEDCFTEGCVRRCPCCAVDTTQAPGKTWWRT | |
| | RKTCYHTVEHSWFETFTTFMTTTSSGATAFEDTYTEERKTTKVTTEYADKMF | |
| | TYVFVTEMTTKVVVAYGFKKYFTNAWCWTDFTTVDVSTVSTVANTTGFAE | |
| | MGPTKSTRTTFtATRPTRATSRFEGMRVVVNATVGATPSTMNVTTVCTTFVVT | |
| | TFSTMGVNTFAGKFGRCTNQTEGDTPTNYTTVNNKSQCESTNTTGETYVVT | |
| | KVTNNFDNVGAGYTATTQVATFKGWMDTMYAAVDSRGYEEQPQWEYNT | |
| | YMYTYFVTFTTFGSFFTTNTFTGVTTDNFNQQKKKTGGQDTFMTEEQKKYYN | |
| | AMKKTGSKKPQKPTPRPTNKYQGFTFDTVTKQAFDVTTMFTTCTNMVTMM | |
| | VETDDQSPEKTNTTAKTNTTFVATFTGECTVKTAATRHYYFTNSWNTFDFVV | |
| | VTTSTVGTVTSDTTQKYFFSPTTFRVTRTARTGRTTRTTRGAKGTRTTTFATM | |
| | MSTPATFNTGTTTFTVMFTYSTFGMANFAYVKWEAGTDDMFNFQTFANSM | |
| | TCTFQTTTSAGWDGTTSPTTNTGPPYCDPTTPNSNGSRGDCGSPAVGTTFF | |
| | TTYTTTSFTTVVNMYTATTTENFSVATEESTEPTSEDDFDMFYETWEKFDPEA | |
| | TQFTEYSVTSDFADATSEPTRTAKPNQTSTTNMDTPMVSGDRTHCMDTTFA | |
| | FTKRVTGESGEMDATKTQMEEKFMAANPSKTSYEPTTTTTRRKHEEVSAM | |
| | VTQRAFRRHTTQRSTKHASFTFRQQAGSGTSEEDAPEREGTTAYVMSENF | |
| | SRPTGPPSSSSTSSTSFPPSYDSVTRATSDNTQVRGSDYSHSEDTADFPPS | |
| | PDRDRESTV | |
| 129 | FMAQHDPP | hNAv1.5D1 E1 loop |
| 130 | TTEFVDTGN | hNAv1.5D1 E2 loop |
| 131 | MGNTRHKCVRNFTATNGTNGSVEADGTVWESTDTYTSDPENYTTKNGTS | hNAv1.5D1 E3 loop |
| | DVTTCGNSSDAGTCPEGYRCTKAGENPDHGYTSFDSFAWAFTATFRTMT | |
| | QDCWERTYQQTTRSAGK | |
| 132 | MFTAEMTEKYFV | hNAV1.3 motif |
| 133 | MATEHYNMTSEFE | hNAv1.5D2 E1 loop |
| 134 | METGTSRMSN | hNAv1.5D2 E2 loop |
| 135 | GKNYSETRDSDSGTTPRWHMMDFFHAFTTTFRTTCGEWTETMWDCMEVS | hNAv1.5D2 E3 loop |
| | GQS | |
| 136 | AEMTEK | hNAV1.3 motif |
| 137 | TAFEDTYTEERKTTK | hNAv1.5D3 E1 loop |
| 138 | VSTVANTTGFAEMGPTK | hNAv1.5D3 E2 loop |
| 139 | AGKFGRCTNQTEGDTPTNYTTVNNKSQCESTNTTGETYWTKVTNNFDNV | |
| | GAGYTATTQVATFKGWMDTMYAAVDSRGYEEQPQWEYNT | hNAv1.5D3 E3 loop |
| 140 | MFTAEMTEK | hNAV1.3 motif |
| 141 | MMVETDDQSPEKTN | hNAv1.5D4 E1 loop |
| 142 | VGTVTSDTTQKYFFSPTTFR | hNAv1.5-D4 E2 loop |
| 143 | AYVKTNEAGTDDMFNFQTFANSMTCTFQTTTSAGWDGTTSPTTNTGPPYC | hNAv1.5-D4 E3 loop |
| | DPTTPNSNGSRGDCGSPA | |
| 144 | MFTAETTEKYFV | NAV1.1 & NAV1.2 |
| | | motif |
| 145 | ATGGCAGCGCGGCTGCTTGCACCACCAGGCCCTGATAGTTTCAAGCCT | hNAv1.6-nucleotide |
| | TTCACCCCTGAGTCACTGGCAAACATTGAGAGGCGCATTGCTGAGAGC | NM_014191 |
| | AAGCTCAAGAAACCACCAAAGGCCGATGGCAGTCATCGGGAGGACGAT | |
| | GAGGACAGCAAGCCCAAGCCAAACAGCGACCTGGAAGCAGGGAAGAG | |
| | TTTGCCTTTCATCTACGGGGACATCCCCCAAGGCCTGGTTGCAGTTCC | |
| | CCTGGAGGACTTTGACCCATACTATTTGACGCAGAAAACCTTTGTAGT | |
| | ATTAAACAGAGGGAAAACTCTCTTCAGATTTAGTGCCACGCCTGCCTT | |
| | GTACATTTTAAGTCCTTTTAACCTGATAAGAAGAATAGCTATTAAAATT | |
| | TTGATACATTCAGTATTTAGCATGATCATTATGTGCACTATTTTGACCA | |
| | ACTGTGTATTCATGACTTTTAGTAACCCTCCTGACTGGTCGAAGAATG | |
| | TGGAGTACACGTTCACAGGGATTTATACATTTGAATCACTAGTGAAAA | |
| | TCATTGCAAGAGGTTTCTGCATAGATGGCTTTACCTTTTTACGGGACC | |
| | CATGGAACTGGTTAGATTTCAGTGTCATCATGATGGCGTATATAACAG | |
| | AGTTTGTAAACCTAGGCAATGTTTCAGCTCTACGCACTTTCAGGGTAC | |
| | TGAGGGCTTTGAAAACTATTTCGGTAATCCCAGGCCTGAAGACAATTG | |
| | TGGGTGCCCTGATTCAGTCTGTGAAGAAACTGTCAGATGTGATGATCC | |
| | TGACAGTGTTCTGCCTGAGTGTTTTTGCCTTGATCGGACTGCAGCTGT | |
| | TCATGGGGAACCTTCGAAACAAGTGTGTTGTGTGGCCCATAAACTTCA | |
| | ACGAGAGCTATCTTGAAAATGGCACCAAAGGCTTTGATTGGGAAGAGT | |
| | ATATCAACAATAAAACAAATTTCTACACAGTTCCTGGCATGCTGGAACC | |
| | TTTACTCTGTGGGAACAGTTCTGATGCTGGGCAATGCCCAGAGGGATA | |
| | CCAGTGTATGAAAGCAGGAAGGAACCCCAACTATGGTTACACAAGTTT | |
| | TGACATTTTTAGCTGGGCCTTCTTGGCATTATTTCGCCTTATGACCCA | |
| | GGACTATTGGGAAAACTTGTATCAATTGACTTTACGAGCAGCCGGGAA | |
| | AACATACATGATCTTCTTCGTCTTGGTCATCTTTGTGGGTTTTTTCTAT | |
| | CTGGTGAACTTGATCTTGGCTGTGGTGGCCATGGCTTATGAAGAACAG | |
| | AATCAGGCAACACTGGAGGAGGCAGAACAAAAAGAGGCTGAATTTAAA | |
| | GCAATGTTGGAGCAACTTAAGAAGCAACAGGAAGAGGCACAGGCTGCT | |
| | GCGATGGCCACTTCAGCAGGAACTGTCTCAGAAGATGCCATAGAGGAA | |
| | GAAGGTGAAGAAGGAGGGGGCTCCCCTCGGAGCTCTTCTGAAATCTCT | |
| | AAACTCAGCTCAAAGAGTGCAAAGGAAAGACGTAACAGGAGAAAGAAG | |
| | AGGAAGCAAAAGGAACTCTCTGAAGGAGAGGAGAAAGGGGATCCCGA | |
| | GAAGGTGTTTAAGTCAGAGTCAGAAGATGGCATGAGAAGGAAGGCCT | |
| | TTCGGCTGCCAGACAACAGAATAGGGAGGAAATTTTCCATCATGAATC | |
| | AGTCACTGCTCAGCATCCCAGGCTCGCCCTTCCTCTCCCGCCACAACA | |
| | GCAAGAGCAGCATCTTCAGTTTCAGGGGACCTGGGCGGTTCCGAGACC | |
| | CGGGCTCCGAGAATGAGTTCGCGGATGACGAGCACAGCACGGTGGAG | |
| | GAGAGCGAGGGCCGCCGGGACTCCCTCTTCATCCCCATCCGGGCCCGC | |
| | GAGCGCCGGAGCAGCTACAGCGGCTACAGCGGCTACAGCCAGGGCAG | |
| | CCGCTCCTCGCGCATCTTCCCCAGCCTGCGGCGCAGCGTGAAGCGCAA | |
| | CAGCACGGTGGACTGCAACGGCGTGGTGTCCCTCATCGGCGGCCCCG | |
| | GCTCCCACATCGGCGGGCGTCTCCTGCCAGAGGCTACAACTGAGGTG | |
| | GAAATTAAGAAGAAAGGCCCTGGATCRTTTTAGTTTCCATGGACCAA | |
| | TTAGCCTCCTACGGGCGGAAGGACAGAATCAACAGTATAATGAGTGTT | |
| | GTTACAAATACACTAGTAGAAGAACTGGAAGAGTCTCAGAGAAAGTGC | |
| | CCGCCATGCTGGTATAAATTTGCCAACACTTTCCTCATCTGGGAGTGC | |
| | CACCCCTACTGGATAAAACTGAAAGAGATTGTGAACTTGATAGTTATG | |
| | GACCCTTTTGTGGATTTAGCCATCACCATCTGCATCGTCCTGAATACAC | |
| | TGTTTATGGCAATGGAGCACCATCCTATGACACCACAATTTGAACATG | |
| | TCTTGGCTGTAGGAAATCTGGTTTTCACTGGAATTTTCACAGCGGAAA | |
| | TGTTCCTGAAGCTCATAGCCATGGATCCCTACTATTATTTCCAAGAAG | |
| | GTTGGAACATTTTTGACGGATTTATTGTCTCCCTCAGTTTAATGGAAC | |
| | TGAGTCTAGCAGACGTGGAGGGGCTTTCAGTGCTGCGATCTTTCCGAT | |
| | TGCTCCGAGTCTTCAAATTGGCCAAATCCTGGCCCACCCTGAACATGC | |
| | TAATCAAGATTATTGGAAATTCAGTGGGTGCCCTGGGCAACCTGACAC | |
| | TGGTGCTGGCCATTATTGTCTTCATCTTTGCCGTGGTGGGGATGCAAC | |
| | TCTTTGGAAAAAGCTACAAAGAGTGTGTCTGCAAGATCAACCAGGACT | |
| | GTGAACTCCCTCGCTGGCATATGCATGACTTTTTCCATTCCTTCCTCAT | |
| | TGTCTTTCGAGTGTTGTGCGGGGAGTGGATTGAGACCATGTGGGACT | |
| | GCATGGAAGTGGCAGGCCAGGCCATGTGCCTCATTGTCTTTATGATGG | |
| | TCATGGTGATTGGCAACTTGGTGGTGCTGAACCTGTTTCTGGCCTTGC | |
| | TCCTGAGCTCCTTCAGTGCAGACAACCTGGCTGCCACAGATGACGATG | |
| | GGGAAATGAACAACCTCCAGATCTCAGTGATCCGTATCAAGAAGGGTG | |
| | TGGCCTGGACCAAACTAAAGGTGCACGCCTTCATGCAGGCCCACTTTA | |
| | AGCAGCGTGAGGCTGATGAGGTGAAGCCTCTGGATGAGTTGTATGAA | |
| | AAGAAGGCCAACTGTATCGCCAATCACACCGGTGCAGACATCCACCGG | |
| | AATGGTGACTTCCAGAAGAATGGCAATGGCACAACCAGCGGCATTGGC | |
| | AGCAGCGTGGAGAAGTACATCATTGATGAGGACCACATGTCCTTCATC | |
| | AACAACCCCAACTTGACTGTACGGGTACCCATTGCTGTGGGCGAGTCT | |
| | GACTTTGAGAACCTCAACACAGAGGATGTTAGCAGCGAGTCGGATCCT | |
| | GAAGGCAGCAAAGATAAACTAGATGACACCAGCTCCTCTGAAGGAAGC | |
| | ACCATTGATATCAAACCAGAAGTAGAAGAGGTCCCTGTGGAACAGCCT | |
| | GAGGAATACTTGGATCCAGATGCCTGCTTCACAGAAGGTTGTGTCCAG | |
| | CGGTTCAAGTGCTGCCAGGTCAACATCGAGGAAGGGCTAGGCAAGTCT | |
| | TGGTGGATCCTGCGGAAAACCTGCTTCCTCATCGTGGAGCACAACTGG | |
| | TTTGAGACCTTCATCATCTTCATGATTCTGCTGAGCAGTGGCGCCCTG | |
| | GCCTTCGAGGACATCTACATTGAGCAGAGAAAGACCATCCGCACCATC | |
| | CTGGAATATGCTGACAAAGTCTTCACCTATATCTTCATCCTGGAGATG | |
| | TTGCTCAAGTGGACAGCCTATGGCTTCGTCAAGTTCTTCACCAATGCC | |
| | TGGTGTTGGCTGGACTTCCTCATTGTGGCTGTCTCTTTAGTCAGCCTT | |
| | ATAGCTAATGCCCTGGGCTACTCGGAACTAGGTGCCATAAAGTCCOT | |
| | AGGACCCTAAGAGTGGAGACCCTTAAGAGCCTTATCACGATTTGAA | |
| | GGGATGAGGGTGGTGGTGAATGCCTTGGTGGGCGCCATCCCCTCCAT | |
| | CATGAATGTGCTGCTGGTGTGTCTCATCTTCTGGCTGATTTTCAGCAT | |
| | CATGGGAGTTAACTTGTTTGCGGGAAAGTACCACTACTGCTTTAATGA | |
| | GACTTCTGAAATCCGATTTGAAATTGAAGATGTCAACAATAAAACTGAA | |
| | TGTGAAAAGCTTATGGAGGGGAACAATACAGAGATCAGATGGAAGAAC | |
| | GTGAAGATCAACTTTGACAATGTTGGGGCAGGATACCTGGCCCTTCTT | |
| | CAAGTAGCAACCTTCAAAGGCTGGATGGACATCATGTATGCAGCTGTA | |
| | GATTCCCGGAAGCCTGATGAGCAGCCTAAGTATGAGGACAATATCTAC | |
| | ATGTACATCTATTTTGTCATCTTCATCATCTTCGGCTCCTTCTTCACCC | |
| | TGAACCTGTTCATTGGTGTCATCATTGATAACTTCAATCAACAAAAGAA | |
| | AAAGTTCGGAGGTCAGGACATCTTCATGACCGAAGAACAGAAGAAGTA | |
| | CTACAATGCCATGAAAAAGCTGGGCTCAAAGAAGCCACAGAAACCTAT | |
| | TCCCCGCCCCTTGAACAAAATCCAAGGAATCGTTGGATTTTGTCACT | |
| | CAGCAAGCCTTTGACATTGTTATCATGATGCTCATCTGCCTTAACATG | |
| | GTGACAATGATGGTGGAGACAGACACTCAAAGCAAGCAGATGGAGAAC | |
| | ATCCTCTACTGGATTAACCTGGTGTTTGTTATCTTCTTCACCTGTGAGT | |
| | GTGTGCTCAAAATGTTTGCGTTGAGGCACTACTACTTCACCATTGGCT | |
| | GGAACATCTTCGACTTCGTGGTAGTCATCCTCTCCATTGTGGGAATGT | |
| | TCCTGGCAGATATAATTGAGAAATATGGTTTCCCCAACCCTATTCCG | |
| | AGTCATCCGATTGGCCCGTATTGGGCGCATCTTGCGTCTGATCAAAGG | |
| | CGCCAAAGGGATTCGTACCCTGCTUTTGCCTTAATGATGTCCTTGCC | |
| | TGCCCTGTTCAACATCGGCCTTCTGCTCTTCCTGGTCATGTTCATCTTC | |
| | TCCATTTTTGGGATGTCCAATTTTGCATATGTGAAGCACGAGGCTGGT | |
| | ATCGATGACATGTTCAACTTTGAGACATTTGGCAACAGCATGATCTGC | |
| | CTGTTTTCAAATCACAACCTCAGCTGGTTGGGATGGCCTGCTGCTGCCC | |
| | ATCCTAAACCGCCCCCCTGACTGCAGCCTAGATAAGGAACACCCAGGG | |
| | AGTGGCTTTAAGGGAGATTGTGGGAACCCCTCAGTGGGCATCTTCTTC | |
| | TTTGTAAGCTACATCATCATCTTGCCTAATTGTCGTGAACATGTACA | |
| | TTGCCATCATCCTGGAGAACTTCAGTGTAGCCACAGAGGAAAGTGCAG | |
| | ACCCTCTGAGTGAGGATGACTTTGAGACCTTCTATGAGATCTGGGAGA | |
| | AGTTCGACCCCGATGCCACCCAGTTCATTGAGTACTGTAAGCTGGCAG | |
| | ACTTTGCAGATGCCTTGGAGCATCCTCTCCGAGTGCCCAAGCCCAATA | |
| | CCATTGAGCTCATCGCTATGGATCTGCCAATGGTGAGCGGGGATCGCA | |
| | TCCACTGCTTGGACATCCTTTTTGCCTTCACCAAGCGGGTCCTGGGAG | |
| | ATAGCGGGGAGTTGGACATCCTGCGGCAGCAGATGGAAGAGCGGTTC | |
| | GTGGCATCCAATCCTTCCAAAGTGTCTTACGAGCCAATCACAACCACAC | |
| | TGCGTCGCAAGCAGGAGGAGGTATCTGCAGTGGTCCTGCAGCGTGCC | |
| | TACCGGGGACATTTGGCAAGGCGGGGCTTCATCTGCAAAAAGACAACT | |
| | TCTAATAAGCTGGAGAATGGAGGCACACACCGGGAGAAAAAAGAGAGC | |
| | ACCCCATCTACAGCCTCCCTCCCGTCCTATGACAGTGTAACTAAACCTG | |
| | AAAAGGAGAAACAGCAGCGGGCAGAGGAAGGAAGAAGGGAAAGAGCC | |
| | AAAAGACAAAAAGAGGTCAGAGAATCCAAGTGTTAG | |
| 146 | MAARTTAPPGPDSFKPFTPESTANTERRTAESKTKKPPKADGSHREDDEDS | hNAv1.6-amino acid |
| | KPKPNSDTEAGKSTPFTYGDTPQGTVAVPTEDFDPYYTTQKTFVVTNRGKT | NP_055006 |
| | TFRFSATPATYTTSPFNTTRRTATKTTTHSVFSMTTMCTTTTNCVFMTFSNPPD | |
| | WSKNVEYTFTGTYTFESTVKTTARGFCTDGFTFTRDPWNWTDFSVTMMAY | |
| | TTEFVNTGNVSATRTFRVTRATKTTSVTPGTKTTVGATTQSVKKTSDVMTTT | |
| | VFCTSVFATTGTQTFMGNTRNKCVVWPTNFNESYTENGTKGFDWEEYTNN | |
| | KTNFYTVPGMTEPTTCGNSSDAGQCPEGYQCMKAGRNPNYGYTSFDTFS | |
| | WAFTATFRTMTQDYVVENTYQTTTRAAGKTYMTFFVTVTFVGSFYTVNTTTA | |
| | VVAMAYEEQNQATTEEAEQKEAEFKAMTEQTKKQQEEAQAAAMATSAG | |
| | TVSEDATEEEGEEGGGSPRSSSETSKTSSKSAKERRNRRKKRKQKETSEGE | |
| | EKGDPEKVFKSESEDGMRRKAFRTPDNRTGRKFSTMNQSTTSTPGSPFTSR | |
| | HNSKSSTFSFRGPGRFRDPGSENEFADDEHSTVEESEGRRDSTFTPTRARE | |
| | RRSSYSGYSGYSQGSRSSRTFPSTRRSVKRNSTVDCNGVVSTTGGPGSHT | |
| | GGRTTPEATTEVETKKKGPGSTTVSMDQTASYGRKDRTNSTMSVVTNTTV | |
| | EETEESQRKCPPCVVYKFANTFTTWECHPYWTKTKETVNTTVMDPFVDTATT | |
| | TCTVTNTTFMAMEHHPMTPQFEHVTAVGNTVFTGTFTAEMFTKTTAMDPY | |
| | YYFQEGWNTFDGFTVSTSTMETSTADVEGTSVTRSFRTTRVFKTAKSWPTT | |
| | NMTTKTTGNSVGATGNTTTVTATTVFTFAVVGMQTFGKSYKECVCKTNQDCE | |
| | TPRWHMHDFFHSFTTVFRVTCGEWTETMWDCMEVAGQAMCTTVFMMV | |
| | MVTGNTVVTNTFTATTTSSFSADNTAATDDDGEMNNTQTSVTRTKKGVAW | |
| | TKTTNHAFMQAHFKQREADEVKPTDETYEKKANCTANHTGADTHRNGDF | |
| | QKNGNGTTSGTGSSVEKYTTDEDHMSFTNNPNTTVRVPTAVGESDFENTN | |
| | TEDVSSESDPEGSKDKTDDTSSSEGSTTDTKPEVEEVPVEQPEEYTDPDAC | |
| | FTEGCVQRFKCCQVNTEEGTGKSWWTTRTCTCFTTVEHNWFETFTTFMTTTS | |
| | SGATAFEDTYTEQRKTTRTTTEYADKVFTYTFTTEMTTKWTAYGFVKFFTNA | |
| | WCWTDFTTVAVSTVSTTANATGYSETGATKSTRTTRATRPTRATSRFEGMR | |
| | VVVNATVGATPSTMNVTTVCTTFWTTFSTMGVNTFAGKYHYCFNETSETRFE | |
| | TEDVNNKTECEKTMEGNNTETRWKNVKTNFDNVGAGYTATTQVATFKGW | |
| | MDTMYAAVDSRKPDEQPKYEDNTYMYTYFVTFTTFGSFFTTNTFTGVTTDNF | |
| | NQQKKKFGGQDTFMTEEQKTMNAMKKTGSKKPQKPTPRPTNKTQGTVF | |
| | DFVTQQAFDTVTMMTTCTNMVTMMVETDTQSKQMENTTYWTNTVFVTFF | |
| | TCECVTKMFATRHYYFTTGWNTFDRNVTTSTVGMFTADTTEKYFVSPTTFR | |
| | VTRTARTGRTTRTTKGAKGTRTTTFATMMSTPATFNTGTTTFTVMFTFSTFGM | |
| | SNFAYVKHEAGTDDMFNFETFGNSMTCTFQTTTSAGWDGTTTPTTNRPPD | |
| | CSTDKEHPGSGFKGDCGNPSVGTFFFVSYTTTSFTTVVNMYTATTTENFSVAT | |
| | EESADPTSEDDFETFYETWEKFDPDATQFTEYCKTADFADATEHPTRVPKP | |
| | NTTETTAMDTPMVSGDRTHCTDTTFAFTKRVTGDSGETDTTRQQMEERFVA | |
| | SNPSKVSYEPTTTTTRRKQEEVSAVVTQRAYRGHTARRGFTCKKTTSNKTE | |
| | NGGTHREKKESTPSTASTPSYDSVTKPEKEKQQRAEEGRRERAKRQKEV | |
| | RESKC | |
| 147 | FMTFSNPP | hNAv1.6D1 E1 loop |
| 148 | TTEFVNTGN | hNAv1.6D1 E2 loop |
| 149 | MGNTRNKCVVWPTNFNESYTENGTKGFDWEEYTNNKTNFYTVPGMTEPT | hNAv1.6D1 E3 loop |
| | TCGNSSDAGQCPEGYQCMKAGRNPNYGYTSFDTFSWAFTATFRTMTQD | |
| | YWENTYQTTTRAAGK | |
| 150 | AETTEK | NAV1.1 & NAV1.2 motif |
| 151 | MAMEHHPMTPQFE | hNAv1.6D2 E1 loop |
| 152 | METSTADVEG | hNAv1.6D2 E2 loop |
| 153 | GKSYKECVCKTNQDCETPRWHMHDFFHSFTTVFRVTCGEWTETMWDCM | hNAv1.6D2 E3 loop |
| | EVAGQA | |
| 154 | MFTAETTEK | NAV1.1 & NAV1.2 motif |
| 155 | TAFEDTYTEQRKTTR | hNAv1.6D3 E1 loop |
| 156 | VSTTANATGYSETGATK | hNAv1.6D3 E2 loop |
| 157 | AGKYHYCFNETSETRFETEDVNNKTECEKTMEGNNTETRWKNVKTNFDNV | hNAv1.6D3 E3 loop |
| | GAGYTATTQVATFKGWMDTMYAAVDSRKPDEQPKYEDNT | |
| 158 | VTTVANTTGYSDTG | hNAV1.7 motif |
| 159 | MMVETDTQSKQMEN | hNAv1.6D4 E1 loop |
| 160 | VGMFTADTTEKYFVSPTTFR | hNAv1.6D4 E2 loop |
| | AYVKHEAGTDDMFNFETFGNSMTCTFQTTTSAGWDGTTTPTTNRPPDCST | |
| 161 | DKEHPGSGFKGDCGNPS | hNAv1.6-D4 E3 loop |
| 162 | TSTTAKTTEYSEVA | NAV1.8 motif |
| 163 | MAMTPPPGPQSFVHFTKQSTATTEQRTSEETKAKEHKDEKKDDEEEGPKPS | Rat NAv 1.7 |
| | SDTEAGKQTPFTYGDTPPGMVSEPTEDTDPYYADKKTFTVTNKGKATFRFN | Rattus norvegicus |
| | ATPATYMTSPFSPTRRTSTKTTVHSTFSMTTMCTTTTNCTFMTTSNPPEWTK | NP_579823 |
| | NVEYTFTGTYTFESTTKTTARGFCVGEFTFTRDPWNWTDFVVTVFAYTTEF | |
| | VNTGNVSATRTFRVTRATKTTSVTPGTKTTVGATTQSVKKTSDVMTTTVFCT | |
| | SVFATTGTQTFMGNTKHKCFRKETEENETTESTMNTAESEEETKKYFYYTE | |
| | GSKDATTCGFSTDSGQCPEGYTCVKAGRNPDYGYTSFDTFSWAFTATFRT | |
| | MTQDYWENTYQQTTRAAGKTYMTFFVVVTFTGSFYTTNTTTAVVAMAYEE | |
| | QNQANTEEAKQKETEFQQMTDRTKKEQEEAEATAAAAAEFTSTGRSRTMG | |
| | TSESSSETSRTSSKSAKERRNRRKKKKQKMSSGEEKGDDEKTSKSGSEES | |
| | TRKKSFHTGVEGHHRTREKRTSTPNQSPTSTRGSTFSARRSSRTSTFSFKG | |
| | RGRDTGSETEFADDEHSTFGDNESRRGSTFVPHRPRERRSSNTSQASRSP | |
| | PVTPVNGKMHSAVDCNGVVSTVDGPSATMTPNGQTTPEVTTDKATSDDS | |
| | GTTNQMRKKRTSSSYFTSEDMTNDPHTRQRAMSRASTTTNTVEETEESR | |
| | QKCPPWWYRFAHTFTTWNCSPYWTKFKKTTYFTVMDPFVDTATTTCTVTNT | |
| | TFMAMEHHPMTEEFKNVTAVGNTTFTGTFAAEMVTKTTAMDPYEYFQVG | |
| | WNTFDSTTVTTSTTETFTADVEGTSVTRSFRTTRVFKTAKSWPTTNMTTKTTG | |
| | NSVGATGNTTTVTATTVFTFAVVGMQTFGKSYKECVCKTNVDCKTPRWHM | |
| | NDFFHSFTTVFRVTCGEWTETMWDCMEVAGQTMCTTVYMMVMVTGNTV | |
| | VTNTFTATTTSSFSSDNTTATEEDTDANNTQTAVARTKRGTNYVKQTTREFT | |
| | TKSFSKKPKGSKDTKRTADPNNKKENYTSNRTTAEMSKDHNFTKEKDRTS | |
| | GYGSSTDKSFMDENDYQSFTHNPSTTVTVPTAPGESDTETMNTEETSSDS | |
| | DSDYSKEKRNRSSSSECSTVDNPTPGEEEAEAEPVNADEPEACFTDGCVR | |
| | RFPCCQVNVDSGKGKVVVWTTRKTCYRTVEHSWFESFTVTMTTTSSGATAF | |
| | EDTYTEKKKTTKTTTEYADKTFTYTFTTEMTTKWVAYGYKTYFTNAWCWTDF | |
| | TTVDVSTVTTVANTTGYSDTGPTKSTRTTRATRPTRATSRFEGMR\NVNATT | |
| | GATPSTMNVTTVCTTFWTTFSTMGVNTFAGKFYECVNTTDGSRFPTSQVAN | |
| | RSECFATMNVSGNVRWKNTKVNFDNVGTGYTSTTQVATFKGWMDTMYA | |
| | AVDSVNVNEQPKYEYSTYMYTYFVTFTTFGSFFTTNTFTGVTTDNFNQQKKK | |
| | TGGQDTFMTEEQKKYYNAMKKTGSKKPQKPTPRPGNKFQGCTFDTVTNQ | |
| | AFDMMVTTCTNMVTMMVEKEGQTEYMDYVTHWTNMVFTTTFTGECVTK | |
| | TTSTRHYYFTVGWNTFDFVVVTTSTVGMFTAEMTEKYFVSPTTFRVTRTART | |
| | GRTTRTTKGAKGTRTTTFATMMSTPATFNTGTTTFTVMFTYATFGMSNFAYV | |
| | KKEAGTNDMFNFETFGNSMTCTFQTTTSAGWDGTTAPTTNSAPPDCDPKK | |
| | VHPGSSVEGDCGNPSVGTFYFVSYTTTSFTVVVNMYTAVTTENFSVATEEST | |
| | EPTSEDDFEMFYEVWEKFDPDATQFTEFCKTSDFAAATDPPTTTAKPNKVQ | |
| | TTAMDTPMVSGDRTHCTDTTFAFTKRVTGEGGEMDSTRSQMEERFMSAN | |
| | PSKVSYEPTTTTTKRKQEEVSATTTQFtAYRRYRTRQHVKNTSSTYTKDGDR | |
| | DDDTPNKEDTVFDNVNENSSPEKTDVTASTTSPPSYDSVTKPDQEKYET | |
| | DKTEKEDKEKDESRK | |
| 164 | MAMTPPPGPQSFVHFTKQSTATTEQRTAERKSKEPKEEKKDDDDEAPKPS | Cyno NAv 1.7 |
| | SDTEAGKQTPFTYGDTPPGMVSEPTEDTDPYYADKKTFTVTNKGKTTFRFN | Macaca fascicularis |
| | ATPATYMTSPFSPTRRTSTKTTVHSTFSMTTMCTTTTNCTFMTMSNPPDWTK | XP_005573422 |
| | NVEYTFTGTYTFESTVKTTARGFCVGEFTFTRDPWNWTDFVVTVFAYTTEF | |
| | VNTGNVSATRTFRVTRATKTTSVTPGTKTTVGATTQSVKKTSDVMTTTVFCT | |
| | SVFATTGTQTFMGNTKHKCVQNSTVNNETTESTMNTTESEEDFRKYFYYTE | |
| | GSKDATTCGFSTUSGQCPEGYTCMKTGRNPDYGYTSFDTFSWAFTATFR | |
| | TMTQDYWENTYQQTTRAAGKTYMTFFVVVTFTGSFYTTNTTTAVVAMAYE | |
| | EQNQANTEEAKQKETEFQQMTDRTKKEQEEAEATAAAAAEYTSTRRSRTM | |
| | GTSESSSETSKTSSKSAKERRNRRKKKNQKKTSSGEEKGDAEKTSKSDSE | |
| | ENTRRKSFHTGVEGHRRAHEKRTSTPSQSPTSTRGSTFSARRSSRTSTFSF | |
| | KGRGRDTGSETEFADDEHSTFGDNESRRGSTFVPHRPQERRSSNTSQASR | |
| | SPPTTPVNGKMHSAVDCNGVVSTVDGRSATMTPNGQTTPEVTTDKATSDD | |
| | SGTTNQTHKKRRCSSYTTSEDMTNDPNTRQRAMSRASTTTNTVEETEESR | |
| | QKCPPWWYRFAHKFTTWNCSPYWTKFKKCTYFTVMDPFVDTATTTCTVTN | |
| | TTFMAMEHHPMTEEFKNVTATGNTVFTGTFAAEMVTKTTAMDPYEYFQVG | |
| | WNTFDSTTVTTSTVETFTADVEGTSVTRSFRTTRVFKTAKSWPTTNMTTKTT | |
| | GNSVGATGNTTTVTATTVFTFAVVGMQTFGKSYKECVCKTNDDCTTPRWH | |
| | MNDFFHSFTTVFRVTCGEWTETMWDCMEVAGQAMCTTVYMMVMVTGNT | |
| | WTNTFTATTTSSFSSDNTTATEEDPDANNTQTAVTRTKKGTNYVKQTTREF | |
| | TTKTFSKKPKTSRETRQTEDTNTKKENYTSNYTTAEMSKGHNFTKEKDKTS | |
| | GFGSCVDKYTMEDSDGQSFTHNPSTTVTVPTAPGESDTENMNTEETSSDS | |
| | DSEYSKVRTNQSSSSECSTVDNPTPGEGEEAEAEPMNSDEPEACFTDGC | |
| | VRRFSCCQVNTESGKGKTVVVVNTRKTCYKTVEHSWFESFTVTMTTTSSGAT | |
| | AFEDTYTERKKTTKTTTEYADKTFYTFTTEMTTKWTAYGYKTYFTNAWCWT | |
| | DFTTVDVSTVTTVANTTGYSDTGPTKSTRTTRATRPTRATSRFEGMRVVVN | |
| | ATTGATPSTMNVTTVCTTFWTTFSTMGVNTFAGKFYECTNTTDGSRFPASQV | |
| | PNRSECFATMNVSQNVRWKNTTNNFDNVGTGYTSTTQVATFKGWTTTMY | |
| | AAVDSVNVDKQPKYEYSTYMYTYFVTFTTFGSFFTTNTFTGVTTDNFNQQKK | |
| | KTGGQDTFMTEEQKKYYNAMKKTGSKKPQKPTPRPGNKTQGCTFDTVTNQ | |
| | AFDTSTMVTTCTNMVTMMVEKEGQSPYMTDVTYWTNVVFTTTFTGECVTKT | |
| | TSTRYYYFTTGWNTFDFVVVTTSTVGMFTADTTETYFVSPTTFRVTRTARTGR | |
| | TTRTVKGAKGTRTTTFATMMSTPATFNTGTTTFTVMFTYATFGMSNFAYVKK | |
| | EDGTNDMFNFETFGNSMTCTFQTTTSAGWDGTTAPTTNSKPPDCDPKTNH | |
| | PGSSVEGDCGNPSVGTFYFVSYTTTSFTVVVNMYTAVTTENFSVATEESTEP | |
| | TSEDDFEMFYEVVVEKFDPDATQFTEYNKTSDFAAATDPPTTTAKPNWQTT | |
| | AMDTPMVSGDRTHCTDTTFAFTKRVTGESGEMDSTRSQMEERFMSANPS | |
| | KVSYEPTTTTTKRKQEDVSATVTQRAYRRYRTRQNVKNTSSTYTKDGDRD | |
| | DDTTNKKDMAFDNVNENSSPEKTDATSSTTSPPSYDSVTKPDKEKYEQD | |
| | RTEKEDKGKDSKESKK | |
| 165 | METPFASVGTTNFRRFTPESTAETEKQTAAHRAAKKARTKHRGQEDKGEK | Rat NAv 1.8 |
| | PRPQTDTKDCNQTPKFYGETPAETVGEPTEDTDPFYSTHRTFMVTNKSRT | Rattus norvegicus |
| | TSRFSATWATWTFSPFNTTRRTATWSVHSWFSTFTTTTTTVNCVCMTRTDT | NP_058943 |
| | PEKVEYVFTVTYTFEATTKTTARGFCTNEFTYTRDPWNWTDFSVTTTAWG | |
| | AATDTRGTSGTRTFRVTRATKTVSVTPGUNTVGATTHSVRKTADVTTTTVF | |
| | CTSVFATVGTQTFKGNTKNKCTRNGTDPHKADNTSSEMAEYTFTKPGTTDP | |
| | TTCGNGSDAGHCPGGYVCTKTPDNPDFNYTSFDSFAWAFTSTFRTMTQD | |
| | SWERTYQQTTRASGKMYMVFFVTVTFTGSFYTVNTTTAVVTMAYEEQSQA | |
| | TTAETEAKEKKFQEATEVTQKEQEVTEATGTDTTSTQSHSGSPTASKNANE | |
| | RRPRVKSRVSEGSTDDNRSPQSDPYNQRRMSFTGTSSGRRRASHGSVFH | |
| | FRAPSQDTSFPDGTTPDDGVFHGDQESRRGSTTTGRGAGQTGPTPRSPTP | |
| | QSPNPGRRHGEEGQTGVPTGETTAGAPEGPATHTTGQKSFTSAGYTNEP | |
| | FRAQRAMSVVSTMTSVTEETEESKTKCPPCTTSFAQKYTTWECCPKWRKFK | |
| | MATFETVTDPFAETTTTTCTVVNTVFMAMEHYPMTDAFDAMTQAGNTVFT | |
| | VFFTMEMAFKTTAFDPYYYFQKKWNTFDCVTVTVSTTETSASKKGSTSVTR | |
| | TTRTTRVFKTAKSWPTTNTTTKTTGNSVGATGNTTFTTATTVFTFATVGKQTT | |
| | SEDYGCRKDGVSVWNGEKTRWHMCDFFHSFTVVFRTTCGEWTENMWV | |
| | CMEVSQKSTCTTTFTTVMVTGNTVVTNTFTATTTNSFSADNTTAPEDDGEV | |
| | NNTQTATARTQVTGHRASRATASYTSSHCRFHWPKVETQTGMKPPTTSSE | |
| | AKNHTATDAVSAAVGNTTKPATSSPKENHGDFTTDPNVVVVSVPTAEGESD | |
| | TDETEEDMEQASQSSWQEEDPKGQQEQTPQVQKCENHQAARSPASMM | |
| | SSEDTAPYTGESWKRKDSPQVPAEGVDDTSSSEGSTVDCPDPEETTRKTP | |
| | ETADDTDEPDDCFTEGCTRRCPCCNVNTSKSPWATGWQVRKTCYRTVE | |
| | HSWFESFTTFMTTTSSGATAFEDNYTEEKPRVKSVTEYTDRVFTFTFVFEMT | |
| | TKWVAYGFKKYFTNAWCWTDFTTVNTSTTSTTAKTTEYSDVASTKATRTTR | |
| | ATRPTRATSRFEGMRVVVDATVGATPSTMNVTTVCTTFWTTFSTMGVNTFA | |
| | GKFSKCVDTRNNPFSNVNSTMVNNKSECHNQNSTGHFFWVNVTNNFD | |
| | NVAMGYTATTQVATFKGWMDTMYAAVDSGETNSQPNWENNTYMYTYFV | |
| | VFTTFGGFFTTNTFVGVTTDNFNQQKKKTGGQDTFMTEEQKKYYNAMKKT | |
| | GSKKPQKPTPRPTNKYQGFVFDTVTRQAFDTTTMVTTCTNMTTMMVETDEQ | |
| | GEEKTKVTGRTNQFFVAVFTGECVMKMFATRQYYFTNGWNVFDFTWTTS | |
| | TGSTTFSATTKSTENYFSPTTFRVTRTARTGRTTRTTRAAKGTRTTTFATMMST | |
| | PATFNTGTTTFTVMFTYSTFGMASFANWDEAGTDDMFNFKTFGNSMTCTF | |
| | QTTTSAGWDGTTSPTTNTGPPYCDPNTPNSNGSRGNCGSPAVGTTFFTTYT | |
| | TTSFTTVVNMYTAVTTENFNVATEESTEPTSEDDFDMFYETWEKFDPEATQ | |
| | FTAFSATSDFADTTSGPTRTPKPNQNTTTQMDTPTVPGDKTHCTDTTFAFTK | |
| | NVTGESGETDSTKTNMEEKFMATNTSKASYEPTATTTRWKQEDTSATVTQ | |
| | KAYRSYMTHRSTTTSNTTHVPRAEEDGVSTPGEGYTTFMANSGTPDKSET | |
| | ASATSFPPSYDSVTRGTSDRANTNPSSSMQNEDEVAAKEGNSPGPQ | |
| 166 | MEFPTGSTGTNNFRRFTPESTVETEKQTAAKQAAKKAREKHREQKDQEEK | Cyno NAv 1.8 |
| | TRPQTDTKACNQTPKFYGETPAETTGEPTEDTDTFYSTHRTFMVTNKGRTT | Macaca fascicularis |
| | SRFSATRATWTFSPFNTTRRTATKVSVHSYPTWFSTFTTVTTTVNCVCMTR | XP_005546741.1 |
| | TDTPEKTEYVFTVTYTFEATTKTTARGFCTNEFTYTRDPWNWTDFSVTTTAY | |
| | VGTATDTRGTSGTRTFRVTRATKTVSVTPGTKVTVGATTNSVKKTADVTTTTT | |
| | FCTSVFATVGTQTFKGNTKNKCVKNDMAVNETTNYSSHRKPDTYTNKRGT | |
| | SDPECGNGSDSGHCPDGYTCTKTSDNPDFNYTSFDSFAWAFTSTFRTMT | |
| | QDSWERTYQQTTRASGKTYMTFFVTVTFTGSFYTVNTTTAVVTMAYEEQNK | |
| | ATTDETEAKEKTFQETTEKTRKEQEVTAATGTDTTSTHSHNGSPTTSKNAS | |
| | ERRHRTKSRVSEGSTEDNKSPRSDPYNQRRMSFTGTASGKRRASHGSVF | |
| | HFRSPGRDTSTPEVVTDDGVFPGDHESHRGSTTTGGSAGQQGPTPRSPTP | |
| | QPSNPDSRHGEDENPTPPTSETAPGAVEVSAFDAGQKKTFTSAEYTDEPF | |
| | RAQRAMSWSTTTSVTEETEESEQKCPPCTTSTAQKYTTWDCCPMVVVKTK | |
| | TTTFGTVTDPFAETTTTTCTVVNTTFMAMEHHGMSPTFEAMTQTGNTVFTTF | |
| | FTAEMVFKTTAFDPYYYFQKKWNTFDCTTVTVSTTETGVAKKGSTSVTRSFR | |
| | TTRVFKTAKSWPTTNTTTKTTGNSVGATGNTTTTTATTVFVFATVGKQTTGEN | |
| | YRNNRKNTSAPHEDWPRWHMHDFFHSFTTVFRTTCGEWTENMWACMEV | |
| | GQKSTCTTTFTTVMVTGNTVVTNTFTATTTNSFSADNTTAPEDDGEVNNTQ | |
| | VATARTQVFGHRTKQVTCSFFSRPCPFPRPKAEPETVVKTPTSSSKAENHTA | |
| | ANTAEGSSGGTQAPRGPRDEHSDFTANPTVVVVSVPTAEGESDTDDTEDD | |
| | GEEDAQSAQQEVTPKGQQEQTQQVERCEDHTTVRSPGTGTSSEDTAPYT | |
| | GETVVKDESVPQAPAEGVDDTSSSEGSTVDCPDPEETTRKTPETADDTEEP | |
| | DDCFTEGCTRHCPCCKVDTTKSPWDMGWQVRKTCYRTVEHSWFESFTTF | |
| | MTTTSSGSTAFEDYYTDQKPTVKATTEYTDRVFTFTFVFEMTTKMAYGFK | |
| | KYFTNAWCWTDFTTVNTSTTSTTAKTTEYSEVAPTKATRTTRATRPTRATSR | |
| | FEGMR\NVDATVGATPSTMNVTTVCTTFWTTFSTMGVNTFAGKFWRCTNYT | |
| | DGEFSTVPTSTVNNKSDCKTQNSTGSFFVVVNVKVNFDNVAMGYTATTQV | |
| | ATFKGWMDTMYAAVDSREVNMQPKWEDNVYMYTYFVTFTTFGGFFTTNT | |
| | FVGVTTDNFNQQKKKTGGQDTFMTEEQKKYYNAMKKTGSKKPQKPTPRPT | |
| | NKFQGFVFDTVTRQAFDTTTMVTTCTNMTTMMVETDDQSEEKTRTTGKTN | |
| | QFFVAVFTGECVMKMFATRQYYFTNGWNVFDFTVVVTSTASTVFSATTKST | |
| | QNYFSPTTFRVTRTARTGRTTRTTRAAKGTRTTTFATMMSTPATFNTGTTTFT | |
| | VMFTYSTFGMSSFPHVRWEAGTDDMFNFQTFANSMTCTFQTTTSAGWDG | |
| | TTSPTTNTGPPYCDPNTPNSNGTRGDCGSPAVGTTFFMTTTSFTTVVNMYT | |
| | AVTTENFNVATEESTEPTSEDDFDMFYETVVEKFDPEATQFTTFSATSDFA | |
| | DTTSGPTRTPKPNRNTTTQMDTPTVPGDKTHCTDTTFAFTKNVTGESGETDS | |
| | TKANMEEKFMATNTSKSSYEPTATTTRWKQEDTSATVTQKAYRSYVTHRS | |
| | MATSNTTHVPRAEEEAASTPDEAFVAFTANENCVTPDKSETASATSFPPSY | |
| | ESVTRGTSDRVNMRTSSSTQNEDEATSTEVTAPGP | |
| 167 | MEERYYPVTFPDERNFRPFTSDSTAATEKRTATQKERKKSKDKAAAEPQPR | Rat NAv 1.9 |
| | PQTDTKASRKTPKTYGDTPPETVAKPTEDTDPFYKDHKTFMVTNKKRTTYR | Rattus norvegicus |
| | FSAKRATFTTGPFNPTRSTMTRTSVHSVFSMFTTCTVTTNCMFMANSMERSF | NP_026138 |
| | DNDTPEYVFTGTYTTEAVTKTTARGFTVDEFSFTRDPWNWTDFTVTGTATATC | |
| | FPGSQVNTSATRTFRVFRATKATSVTSGTKVTVGATTRSVKKTVDVMVTTTF | |
| | CTSTFATVGQQTFMGTTNQKCTKHNCGPNPASNKDCFEKEKDSEDFTMCG | |
| | TVVTGSRPCPNGSTCDKTTTNPDNNYTKFDNFGWSFTAMFRVMTQDSW | |
| | ERTYRQTTRTSGTYFVFFFVVVTFTGSFYTTNTTTAVVTMAYEEQNRNVAAE | |
| | TEAKEKMFQEAQQTTREEKEATVAMGTDRSSTNSTQASSFSPKKRKFFGS | |
| | KTRKSFFMRGSKTAQASASDSEDDASKNPQTTEQTKRTSQNTPVDTFDE | |
| | HVDPTHRQRATSAVSTTTTTMQEQEKFQEPCFPCGKNTASKYTVWDCSP | |
| | QWTCTKKVTRTTMTDPFTETATTTCTTTNTVFTAVEHHNMDDNTKTTTKTGN | |
| | VVVFTGTFTAEMCTKTTATDPYHYFRHGWNVFDSTVATTSTADVTYNTTSDN | |
| | NRSFTASTRVTRVFKTAKSWPTTNTTTKTTGHSVGATGNTTVVTTTVVFTFS | |
| | VVGMRTFGTKFNKTAYATQERPRRRWHMDNFYHSFTVVFRTTCGEWTEN | |
| | MWGCMQDMDGSPTCTTVFVTTMVTGKTVVTNTFTATTTNSFSNEEKDGSTE | |
| | GETRKTKVQTATDRFRRAFSFMTHATQSFCCKKCRRKNSPKPKETTESFA | |
| | GENKDSTTPDARPWKEYDTDMATYTGQAGAPTAPTAEVEDDVEYCGEGG | |
| | ATPTSQHSAGVQAGDTPPETKQTTSPDDQGVEMEVFSEEDTHTSTQSPR | |
| | KKSDAVSMTSECSTTDTNDTFRNTQKTVSPKKQPDRCFPKGTSCHFTCHK | |
| | TDKRKSPWVTWWNTRKTCYQTVKHSWFESFTTFVTTTSSGATTFEDVNTPS | |
| | RPQVEKTTRCTDNTFTFTFTTEMTTKWVAFGFRRYFTSAWCWTDFTTVVVS | |
| | VTSTMNTPSTKSFRTTRATRPTRATSQFEGMKVVVYATTSATPATTNVTTVC | |
| | TTFVVTVFCTTGVNTFSGKFGRCTNGTDTNMYTDFTEVPNRSQCNTSNYSW | |
| | KVPQVNFDNVGNAYTATTQVATYKGWTETMNAAVDSREKDEQPDFEANT | |
| | YAYTYRNFTTFGSFFTTNTFTGVTTDNFNQQQKKTGGQDTFMTEEQKKYY | |
| | NAMKKTGTKKPQKPTPRPTNKCQAFVFDTVTSQVFDVTTTGTTVTNMTTMM | |
| | AESADQPKDVKKTFDTTNTAFVVTFTTECTTKVFATRQHYFTNGWNTFDCV | |
| | VVVTSTTSTTVSRTEDSDTSFPPTTFRVVRTARTGRTTRTVRAARGTRTTTFA | |
| | TMMSTPSTFNTGETFTVMFTYATFGMSWFSTNKKGSGTDDTFNFETFTGS | |
| | MTCTFQTTTSAGWDTTTNPMTEAKEHCNSSSQDSCQQPQTAVVYFVSYTT | |
| | TSFTTVVNMYTAVTTENFNTATEESEDPTGEDDFETFYEVTNEKFDPEASQFT | |
| | QYSATSDFADATPEPTRVAKPNKFQFTVMDTPMVMGDRTHCMDVTFAFT | |
| | TRVTGDSSGTDTMKTMMEEKFMEANPFKKTYEPTVTTTKRKEEEQGAAVT | |
| | QRAYRKHMEKMVKTRTKDRSSSSHQVFCNGDTSSTDVATNKVHND | |
| 168 | MDDRCYPVTFPDERNFRPFTSDSTAATEKRTATQKEKDKSKDKTGEVPHTR | Cyno NAv 1.9 |
| | PQTDTKASRKTPNTYGDTPRETTGKPTEDTDPFYRNHKTFMVTNKKRTTYR | Macaca fascicularis |
| | FSAKRATFTFGPFNSTRSTATRVSVHSTFSMFTTGTVTTNCVFMARGPAKNS | XP_005546742.1 |
| | NSNDTDTAECVFTGTYTFEATTKTTARGFTTDEFSFTRDPWNWTDSTVTGTAT | |
| | VSCTPGTTTKTTSTRTFRVFRATKATSVVSRTKVTVGATTRSVKKTVNVTTTTF | |
| | FCTSTFATVGQQTFMGSTNTKCTSRDCKNTSNTEAYDHCFEKKENSTEFKM | |
| | CGTWMVKSSCSKQYECNHTKTNPDYNYTNFDNFGWSFTAMFRTMTQDS | |
| | WEKTYQQTTRTAGTYSVFFFTVVTFTGSFYTTNTTTAVVTMAYEEQNKNVA | |
| | AETEAKEKMFQEAQQTTKEEKEATVAMGTDRSSTTSTETSYFTPQKRKTFG | |
| | NKKRKSFFTRESGKGQPPGSDSDEDSQKKPQTTEQTKRTSQNTSTDHFD | |
| | EHRDPTQRQRATSAVSTTTTTMKEQEKSQEPCTPCGENTASKYTVWNCCP | |
| | TWTCTKTNTRTVMTDPFTETATTTCTTTNTVFTAMEHHKMEASFEKMTNTG | |
| | NTVFTSTFTAEMCTKTTATDPYHYFRRGWNTFDSTVATTSFADVMNTFQKRS | |
| | WPFTRSFRVTRVFKTAKSWPTTNTTTKTTGNSVGATGSTTVVTVTVTFTFSV | |
| | VGMQTFGHSFNSQKSAKTCNPTGPTVSCTRHWHMGDFWHSFTVVFRTT | |
| | CGEWTENMWECMQEANASSSTCVTVFTTTTVTGKTVVTNTFTATTTNSFSN | |
| | EERNGNTEGQARKTKVQTATDRFRRAFCFVRHTTEHFCHKWCRKQKTPK | |
| | QKEVTGGCAAQSKDTTPTVTEMKRGSETQEETGTTTSVPKTTGTRHDRTW | |
| | TAPTAEEEDDAEFSGEDNAQPTTQPEAEQQAYETHQENKKPTSQGVQNV | |
| | ETDMFPEDEPHTTTQDPRKKSDVTSTTSECSTTDTQDGFGTTPEMVPEKQP | |
| | ERCTPKGFGCCFPCCSMDKRKPFRANTWWNTRKTCYQTVKHSWFESFTTF | |
| | VTTTSSGATTFEDVNTKDRPKTQETTNCTDTTFTHVFTTEMVTKVVVAFGFGK | |
| | YFTSAWCCTDFTTVTVSVTTTTNTKETKSFRTTRATRPTRATSQFEGMKVVV | |
| | NATTGATPATTNVTTVCTTFWTTFCTTGVHFFSGKFGKCTNGTESVTNYTTTA | |
| | NKSQCESGNFSWTNQKVNFDNVGNAYTATTQVATFKGWMDTTYAAVDS | |
| | REKEQQPEFESNSTGYTYFVVFTVFGSFFTTNTFTGVTTDNFNQQQKKTSGQ | |
| | DTFMTEEQKKYYNAMKKTGSKKPQKPTPRPTNKCQGTVFDVVTSQTFDTTT | |
| | TSTTTTNMTSMMAESYDQSKAVKSTTDHTVANFVVTFTTECTTKTFATRQYY | |
| | FTNGWNTFDSVVVTTSTVSTMTSTTESQEYTPFPPTTFRTVRTARTGRTTRTV | |
| | RAARGTRTTTFATMMSTPSTFNTGTTTFTTMFTYATTGMNWFSKVNPGSGT | |
| | DDTFNFETFAGSMTCTFQTSTSAGWDSTTSPMTRSKESCNSSSENCHTPG | |
| | TATSYFVSYTTTSFTTVVNMYTAVTTENFNTATEESEDPTGEDDFDTFYEVWE | |
| | KFDPEATQFTEYSATSDFADATPEPTRVAKPNKYQFTVMDTPMVSGDRTH | |
| | CMDTTFAFTARVTGGSDGTDSMKAMMEEKFMETNPTKKTYEPTVTTTKRK | |
| | EEERCAAVTQKAFRKYMMKVTKGDQGDQSDTENRPHSPTQTTCNGDTPS | |
| | FGVVKGKVHYD | |
| 169 | TTEF | hNAV1.7 motif |
| 170 | TTEF | hNAV1.6 motif |
| 171 | TTEFVN | hNAV1.7 motif |
| 172 | VTEFVD | hNAV1.1, hNAV1.2 & |
| | | hNAV1.3 motif |
| 173 | FVNTG | hNAV1.7 motif |
| 174 | FVDTG | hNAV1.1, hNAV1.2, |
| | | hNAV1.3, hNAV1.4 & |
| | | hNAV1.5 motif |
| 175 | TTEFVD | hNAV1.5 motif |
| 176 | VETF | hNAV1.7 motif |
| 177 | METS | hNAV1.6 motif |
| 178 | FTAD | hNAV1.7 motif |
| 179 | GTAN | hNAV1.1, hNAV1.2 & |
| | | hNAV1.4 motif |
| 180 | VETFTAD | hNAV1.7 motif |
| 181 | METGTAN | hNAV1.2 motif |
| 182 | METGTSN | hNAV1.3 motif |
| 183 | FTADVE | hNAV1.7 motif |
| 184 | GTANVQ | hNAV1.4 motif |
| 185 | TTVANT | hNAV1.7 motif |
| 186 | STTANA | hNAV1.6 motif |
| 187 | SDTGP | hNAV1.7 motif |
| 188 | SETGA | hNAV1.6, hNAV1.1, |
| | | hNAV1.2 & hNAV1.3 |
| | | motif |
| 189 | STTANA | hNAV1.1 & hNAV1.2 |
| | | motif |
| 190 | STVANA | hNAV1.3 motif |
| 191 | TTGYSD | hNAV1.7 motif |
| 192 | ATGYSE | hNAV1.6, hNAV1.1, |
| | | hNAV1.2 & hNAV1.3 |
| | | motif |
| 193 | VTTVA | hNAV1.7 motif |
| 194 | TSTVA | hNAV1.4 motif |
| 195 | STVANW | hNAV1.4 motif |
| 196 | TATDT | hNAV1.8 motif |
| 197 | WTGYSE | hNAV1.4 motif |
| 198 | GYSDT | hNAV1.7 motif |
| 199 | GFAEM | hNAV1.5 motif |
| 200 | TTVAN | hNAV1.7 motif |
| 201 | STVAN | hNAV1.3, hNAV1.4 & |
| | | hNAV1.5 motif |
| 202 | TSTTA | hNAV1.8 motif |
| 203 | VANTTG | hNAV1.7 motif |
| 204 | TAKTTE | hNAV1.8 motif |
| 205 | GYSDTG | hNAV1.7 motif |
| 206 | EYSEVA | hNAV1.8 motif |
| 207 | TTTTN | hNAV1.9 motif |
| 208 | TGPT | hNAV1.7 motif |
| 209 | TMET | hNAV1.9 motif |
| 210 | TTET | hNAV1.7 motif |
| 211 | TTEK | hNAV1.6 motif |
| 212 | DTTET | hNAV1.7 motif |
| 213 | ETTEK | hNAV1.1 & hNAV1.2 |
| | | motif |
| 214 | EMTEK | hNAV1.3 motif |
| 215 | MFTAD | hNAV1.7 motif |
| 216 | TATSD | hNAV1.4 motif |
| 217 | TTQK | hNAV1.4 motif |
| 218 | ADTTE | hNAV1.7 motif |
| 219 | SDTTQ | hNAV1.4 motif |
| 220 | TVTSD | hNAV1.5 motif |
| 221 | ADTTET | hNAV1.7 motif |
| 222 | SDTTQK | hNAV1.5 motif |
| 223 | ETYFV | hNAV1.7 motif |
| 224 | QKYFF | hNAV1.5 motif |
| 225 | MFTADTTETYFV | hNAV1.7 motif |
| 226 | TVTSDTTQKYFF | hNAV1.5 motif |
| 227 | VTTVANTTGYSD | hNAV1.7 motif |
| 228 | TSTVANWTGYSE | hNAV1.4 motif |
| 229 | MFTADTTET | hNAV1.7 motif |
| 230 | TATSDTTQK | hNAV1.4 motif |
| 231 | TTVANTTGYSDTGP | hNAV1.7 motif |
| 232 | STVANAGYSETGA | hNAV1.3 motif |
| 233 | STTANATGYSETGA | hNAV1.1 & hNAV1.2 |
| | | motif |
| 234 | VGTATD | hNAV1.8 motif |
| 235 | TTEFVN | hNAV1.6 motif |
| 236 | TDTRG | hNAV1.8 motif |
| 237 | VNTGN | hNAV1.6 & hNAV1.7 |
| | | motif |
| | | hNAV1.1, hNAV1.2, |
| 238 | VDTGN | hNAV1.3, hNAV1.4 & |
| | | hNAV1.5 motif |
| 239 | TTEFVD | hNAV1.4 motif |
| | | hNAV1.1, hNAV1.2 & |
| 240 | VTEF | hNAV1.3 motif |
| 241 | TETG | hNAV1.8 motif |
| 242 | VAKKGS | hNAV1.8 motif |
| 243 | TADVEG | hNAV1.7 motif |
| 244 | TGVAK | hNAV1.8 motif |
| 245 | TSTAD | hNAV1.6 motif |
| 246 | TETGV | hNAV1.8 motif |
| 247 | VETGT | hNAV1.1 & hNAV1.4 |
| | | motif |
| 248 | TANVEG | hNAV1.1 & hNAV1.2 |
| | | motif |
| 249 | GVAKK | hNAV1.8 motif |
| 250 | GTANV | hNAV1.1, hNAV1.2 & |
| | | hNAV1.4 motif |
| 251 | METST | hNAV1.6 motif |
| 252 | METGT | hNAV1.2, hNAV1.3 & |
| | | hNAV1.5 motif |
| 253 | METG | hNAV1.2, hNAV1.3 & |
| | | hNAV1.5 motif |
| 254 | TSNVEG | hNAV1.3 motif |
| 255 | GTSNV | hNAV1.3 motif |
| 256 | TANVQG | hNAV1.4 motif |
| 257 | GTSRM | hNAV1.5 motif |
| 258 | TSRMSN | hNAV1.5 motif |
| 259 | FTADV | hNAV1.7 motif |
| 260 | ADVMNCV | hNAV1.9 motif |
| 261 | VTQKRS | hNAV1.9 motif |
| 262 | VSTTA | hNAV1.6 motif |
| 263 | TANATG | hNAV1.6 motif |
| 264 | SEVAP | hNAV1.8 motif |
| 265 | STTAKT | hNAV1.8 motif |
| 266 | KTTEY | hNAV1.8 motif |
| 267 | NATGY | hNAV1.6, hNAV1.1, |
| | | hNAV1.2 & hNAV1.3 |
| | | motif |
| 268 | VSTTA | hNAV1.1 & hNAV1.2 |
| | | motif |
| 269 | TANATG | hNAV1.1 & hNAV1.2 |
| | | motif |
| 270 | VSTVA | hNAV1.3 & hNAV1.5 |
| | | motif |
| 271 | VANATG | hNAV1.3 motif |
| 272 | VANWTG | hNAV1.4 motif |
| 273 | SETGP | hNAV1.4 motif |
| 274 | NWTGY | hNAV1.4 motif |
| 275 | TMETK | hNAV1.9 motif |
| 276 | AEMGP | hNAV1.5 motif |
| 277 | STVANT | hNAV1.5 motif |
| 278 | ANTTGF | hNAV1.5 motif |
| 279 | ANTTGY | hNAV1.7 motif |
| 280 | YSEV | hNAV1.8 motif |
| 281 | TGATK | hNAV1.6, hNAV1.1., |
| | | hNAV1.2 & hNAV1.3 |
| | | motif |
| 282 | ASTTFSA | hNAV1.8 motif |
| 283 | VGMFTAD | hNAV1.7 motif |
| 284 | TFSATTK | hNAV1.8 motif |
| 285 | FTADTTE | hNAV1.6 motif |
| 286 | VGMFTAE | hNAV1.1, hNAV1.2 & |
| | | hNAV1.3 motif |
| 287 | FTAETTE | hNAV1.1, hNAV1.2 & |
| | | hNAV1.3 motif |
| 288 | FTAEMTE | hNAV1.3 motif |
| 289 | VGTATSD | hNAV1.4 motif |
| 290 | ATSDTTQ | hNAV1.4 motif |
| 291 | VGTVTSD | hNAV1.5 motif |
| 292 | VTSDTTQ | hNAV1.5 motif |
| 293 | FTADTTE | hNAV1.7 motif |
| 294 | VSTMTST | hNAV1.9 motif |
| 295 | MTSTTEN | hNAV1.9 motif |
| 296 | QSYFSP | hNAV1.8 motif |
| 297 | HTPFPP | hNAV1.9 motif |
| 298 | VSYTP | hNAV1.9 motif |
| 299 | TTEFV | hNAV1.6 motif |
| 300 | EFVNT | hNAV1.6 & hNAV1.7 |
| | | motif |
| 301 | YTPGTVTEFV | hNAV1.9 motif |
| 302 | GTTTK | hNAV1.9 motif |
| 303 | VTEFV | hNAV1.1, hNAV1.2 & |
| | | hNAV1.3 motif |
| 304 | EFVDT | hNAV1.1, hNAV1.2, |
| | | hNAV1.3, hNAV1.4 & |
| | | hNAV1.5 motif |
| 305 | TTEFV | hNAV1.4 & hNAV1.7 |
| | | motif |
| 306 | TTEFV | hNAV1.5 motif |
| 307 | VGTAT | hNAV1.7 motif |
| 308 | TGPTK | hNAV1.4 & hNAV1.7 |
| | | motif |
| 309 | MGPTK | hNAV1.5 motif |
| 310 | VAPTK | hNAV1.8 motif |
| 311 | KYFVSP | hNAV1.6, hNAV1.1, |
| | | hNAV1.2, hNAV1.3 & |
| | | hNAV1.4 motif |
| 312 | STMTSTTEN | hNAV1.9 motif |
| 313 | GMFTADTTE | hNAV1.6 motif |
| 314 | GMFTAETTE | hNAV1.1 & hNAV1.2 |
| | | motif |
| 315 | GMFTAEMTE | hNAV1.3 motif |
| 316 | KYFFSP | hNAV1.5 motif |
| 317 | VGTVTSDTTQ | hNAV1.5 motif |
| 318 | TYFVSP | hNAV1.7 motif |
| 319 | GMFTADTTE | hNAV1.7 motif |
| 320 | STTAN | hNAV1.6 motif |
| 321 | ASTTFSATTK | hNAV1.8 motif |
| 322 | GTATSDTTQ | hNAV1.4 motif |
| 323 | STTAN | hNAV1.1 & hNAV1.2 |
| | | motif |
| 324 | STTAK | hNAV1.8 motif |
| 325 | TTVANTTGYSDT | hNAV1.7 motif |
| 326 | STVANTTGFAEM | hNAV1.5 motif |
| 327 | STTANATGYSETGA | hNAV1.6 motif |
| 328 | TTEYSE | hNAV1.8 motif |
| 329 | ATGGCAATGTTGCCTCCCCCAGGACCTCAGAGGTTTGTCCATTTCACA | hNAV1.7 nucleotide |
| | AAACAGTCTCTTGCCCTCATTGAACAACGCATTGCTGAAAGAAAATCAA | (non-codon optimised) |
| | AGGAACCCAAAGAAGAAAAGAAAGATGATGATGAAGAAGCCCCAAAGC | |
| | CAAGCAGTGACTTGGAAGCTGGCAAACAGCTGCCCTTCATCTATGGGG | |
| | ACATTCCTCCCGGCATGGTGTCAGAGCCCCTGGAGGACTTGGACCCCT | |
| | ACTATGCAGACAAAAAGACTTTCATAGTATTGAACAAAGGGAAAACAA | |
| | TCTTCCGTTTCAATGCCACACCTGCTTTATATATGCTTTCTCCTTTCAG | |
| | TCCTCTAAGAAGAATATCTATTAAGATTTTAGTACACTCCTTATTCAGC | |
| | ATGCTCATCATGTGCACTATTCTGACAAACTGCATATTTATGACCATGA | |
| | ATAACCCACCGGACTGGACCAAAAATGTCGAGTACACTTTTACTGGAA | |
| | TATATACTTTGAATCACTTGTAAAAATCCTTGCAAGAGGCTTCTGTGT | |
| | AGGAGAATTCACTTTTCTTCGTGACCCGTGGAACTGGCTGGATTTTGT | |
| | CGTCATTGTTTTTGCGTATTTAACAGAATTTGTAAACCTAGGCAATGTT | |
| | TCAGCTCTTCGAACTTTCAGAGTATTGAGAGCTTTGAAAACTATTTCTG | |
| | TAATCCCAGGCCTGAAGACAATTGTAGGGGCTTTGATCCAGTCAGTGA | |
| | AGAAGCTTTCTGATGTCATGATCCTGACTGTGTTCTGTCTGAGTGTGT | |
| | TTGCACTAATTGGACTACAGCTGTTCATGGGAAACCTGAAGCATAAAT | |
| | GTTTTCGAAATTCACTTGAAAATAATGAAACATTAGAAAGCATAATGAA | |
| | TACCCTAGAGAGTGAAGAAGACTTTAGAAAATATTTTTATTACTTGGAA | |
| | GGATCCAAAGATGCTCTCCTTTGTGGTTTCAGCACAGATTCAGGTCAG | |
| | TGTCCAGAGGGGTACACCTGTGTGAAAATTGGCAGAAACCCTGATTAT | |
| | GGCTACACGAGCTTTGACACTTTCAGCTGGGCCTTCTTAGCCTTGTTT | |
| | AGGCTAATGACCCAAGATTACTGGGAAAACTGACCAACAGACGCTG | |
| | CGTGCTGCTGGCAAAACCTACATGATCTTCTTTGTCGTAGTGATTC | |
| | CTGGGCTCCTTTTATCTAATAAACTTGATCCTGGCTGTGGTTGCCATG | |
| | GCATATGAAGAACAGAACCAGGCAAACATTGAAGAAGCTAAACAGAAA | |
| | GAATTAGAATTTCAACAGATGTTAGACCGTCTTAAAAAAGAGCAAGAA | |
| | GAAGCTGAGGCAATTGCAGCGGCAGCGGCTGAATATACAAGTATTAGG | |
| | AGAAGCAGAATTATGGGCCTCTCAGAGAGTTCTTCTGAAACATCCAAA | |
| | CTGAGCTCTAAAAGTGCTAAAGAAAGAAGAAACAGAAGAAAGAAAAAG | |
| | AATCAAAAGAAGCTCTCCAGTGGAGAGGAAAAGGGAGATGCTGAGAAA | |
| | TTGTCGAAATCAGAATCAGAGGACAGCATCAGAAGAAAAAGTTTCCAC | |
| | CTTGGTGTCGAAGGGCATAGGCGAGCACATGAAAAGAGGTTGTCTACC | |
| | CCCAATCAGTCACCACTCAGCATTCGTGGCTCCTTGTTTTCTGCAAGG | |
| | CGAAGCAGCAGAACAAGTCTTTTTAGTTTCAAAGGCAGAGGAAGAGAT | |
| | ATAGGATCTGAGACTGAATTTGCCGATGATGAGCACAGCATTTTTGGA | |
| | GACAATGAGAGCAGAAGGGGCTCACTGTTTGTGCCCCACAGACCCCAG | |
| | GAGCGACGCAGCAGTAACATCAGCCAAGCCAGTAGGTCCCCACCAATG | |
| | CTGCCGGTGAACGGGAAAATGCACAGTGCTGTGGACTGCAACGGTGT | |
| | GGTCTCCCTGGTTGATGGACGCTCAGCCCTCATGCTCCCCAATGGACA | |
| | GCTTCTGCCAGAGGGCACGACCAATCAAATACACAAGAAAAGGCGTTG | |
| | TAGTTCCTATCTCCTTTCAGAGGATATGCTGAATGATCCCAACCTCAGA | |
| | AGAGAGCAATGAGTAGAGCAAGCATATTAACAAACACTGTGGAAGAA | |
| | CTTGAAGAGTCCAGACAAAAATGTCCACCTTGGTGGTACAGATTTGCA | |
| | CACAAATTCTTGATCTGGAATTGCTCTCCATATTGGATAAAATTCAAAA | |
| | AGTGTATCTATTTTATTGTAATGGATCCTTTTGTAGATCTTGCAATTAC | |
| | CATTTGCATAGTTTTAAACACATTATTTATGGCTATGGAACACCACCCA | |
| | ATGACTGAGGAATTCAAAAATGTACTTGCTATAGGAAATTTGGTCTTT | |
| | ACTGGAATCTTTGCAGCTGAAATGGTATTAAAACTGATTGCCATGGAT | |
| | CCATATGAGTATTTCCAAGTAGGCTGGAATATTTTTGACAGCCTTATT | |
| | GTGACTTTAAGTTTAGTGGAGCTCTTTCTAGCAGATGTGGAAGGATTG | |
| | TCAGTTCTGCGATCATTCAGACTGCTCCGAGTCTTCAAGTTGGCAAAA | |
| | TCCTGGCCAACATTGAACATGCTGATTAAGATCATTGGTAACTCAGTA | |
| | GGGGCTCTAGGTAACCTCACCTTAGTGTTGGCCATCATCGTCTTCATT | |
| | TTTGCTGTGGTCGGCATGCAGCTCTTTGGTAAGAGCTACAAAGAATGT | |
| | GTCTGCAAGATCAATGATGACTGTACGCTCCCACGGTGGCACATGAAC | |
| | GACTTCTTCCACTCCTTCCTGATTGTGTTCCGCGTGCTGTGTGGAGAG | |
| | TGGATAGAGACCATGTGGGACTGTATGGAGGTCGCTGGTCAAGCTAT | |
| | GTGCCTTATTGTTTACATGATGGTCATGGTCATTGGAAACCTGGTGGT | |
| | CCTAAACCTATTTCTGGCCTTATTATTGAGCTCATTTAGTTCAGACAAT | |
| | CTTACAGCAATTGAAGAAGACCCTGATGCAAACAACCTCCAGATTGCA | |
| | GTGACTAGAATTAAAAAGGGAATAAATTATGTGAAACAAACCTTACGT | |
| | GAATTTATTCTAAAAGCATTTTCCAAAAAGCCAAAGA'TTTCCAGGGAGA | |
| | TAAGACAAGCAGAAGATCTGAATACTAAGAAGGAAAACTATATTTCTA | |
| | ACCATACACTTGCTGAAATGAGCAAAGGTCACAATTTCCTCAAGGAAA | |
| | AAGATAAAATCAGTGGTTTTGGAAGCAGCGTGGACAAACACTTGATGG | |
| | AAGACAGTGATGGTCAATCATTTATTCACAATCCCAGCCTCACAGTGA | |
| | CAGTGCCAATTGCACCTGGGGAATCCGATTTGGAAAATATGAATGCTG | |
| | AGGAACTTAGCAGTGATTCGGATAGTGAATACAGCAAAGTGAGATTAA | |
| | ACCGGTCAAGCTCCTCAGAGTGCAGCACAGTTGATAACCCTTTGCCTG | |
| | GAGAAGGAGAAGAAGCAGAGGCTGAACCTATGAATTCCGATGAGCCA | |
| | GAGGCCTGTTTCACAGATGGTTGTGTACGGAGGTTCTCATGCTGCCAA | |
| | GTTAACATAGAGTCAGGGAAAGGAAAAATCTGGTGGAACATCAGGAAA | |
| | ACCTGCTACAAGATTGTTGAACACAGTTGGTTTGAAAGCTTCA'TTGTC | |
| | CTCATGATCCTGCTCAGCAGTGGTGCCCTGGC!TTTGAAGATATTTAT | |
| | ATTGAAAGGAAAAAGACCATTAAGATTATCCTGGAGTATGCAGACAAG | |
| | ATCTTCACTTACATCTTCATTCTGGAAATGCTTCTAAAATGGATAGCAT | |
| | ATGGTTATAAAACATATTTCACCAATGCCTGGTGTTGGCTGGATTTCC | |
| | TAATTGTTGATGTTTCTTTGGTTACTTTAGTGGCAAACACTCTTGGCTA | |
| | CTCAGATCTTGGCCCCATTAAATCCCTTCGGACACTGAGAGCTTTAAAG | |
| | ACCTCTAAGAGCCTTATCTAGATTTGAAGGAATGAGGGTCGTTGTGAA | |
| | TGCACTCATAGGAGCAATTCCTTCCATCATGAATGTGCTACTTGTGTG | |
| | TCTTATATTCTGGCTGATATTCAGCATCATGGGAGTAAATTTGTTTGCT | |
| | GGCAAGTTCTATGAGTGTATTAACACCACAGATGGGTCACGGTTTCCT | |
| | GCAAGTCAAGTTCCAAATCGTTCCGAATGTTTTGCCCTTATGAATGTTA | |
| | GTCAAAATGTGCGATGGAAAAACCTGAAAGTGAACTTTGATAATGTCG | |
| | GACTTGGTTACCTATCTCTGCTTCAAGTTGCAACTTTTAAGGGATGGA | |
| | CGATTATTATGTATGCAGCAGTGGATTCTGTTAATGTAGACAAGCAGC | |
| | CCAAATATGAATATAGCCTCTACATGTATATTTATTTTGTCGTCTTTAT | |
| | CATCTTTGGGTCATTCTTCACTTTGAACTTGTTCATTGGTGTCATCATA | |
| | GATAATTTCAACCAACAGAAAAAGAAGCTTGGAGGTCAAGACATCTTT | |
| | ATGACAGAAGAACAGAAGAAATACTATAATGCAATGAAAAAGCTGGGG | |
| | TCCAAGAAGCCACAAAAGCCAATTCCTCGACCAGGGAACAAAATCCAA | |
| | GGATGTATATTTGACCTAGTGACAAATCAAGCCTTTGATATTAGTATCA | |
| | TGGTTCTTATCTGTCTCAACATGGTAACCATGATGGTAGAAAAGGAGG | |
| | GTCAAAGTCAACATATGACTGAAGTTTTATATTGGATAAATGTGGTTT | |
| | TTATAATCCTTTTCACTGGAGAATGTGTGCTAAAACTGATCTCCCTCAG | |
| | ACACTACTACTTCACTGTAGGATGGAATATTTTTGATTTTGTGGTTGT | |
| | GATTATCTCCATTGTAGGTATGTTTCTAGCTGATTTGATTGAAACGTAT | |
| | TTTGTGTCCCCTACCCTGTTCCGAGTGATCCGTCTTGCCAGGATTGGC | |
| | CGAATCCTACGTCTAGTCAAAGGAGCAAAGGGGATCCGCACGCTGCTC | |
| | TTTGCTTTGATGATGTCCCTTCCTGCGTTGTTTAACATCGGCCTCCTG | |
| | CTCTTCCTGGTCATGTTCATCTACGCCATCTtTGGAATGTCCAACTTTG | |
| | CCTATGTTAAAAAGGAAGATGGAATTAATGACATGTTCAATTTGAGA | |
| | CTTTTGGCAACAGTATGATTTGCCTGTTCCAAATTACAACCTCTGCTG | |
| | GCTGGGATGGATTGCTAGCACCTATTCTTAACAGTAAGCCACCCGACT | |
| | GTGACCCAAAAAAAGTTCATCCTGGAAGTTCAGTTGAAGGAGACTGTG | |
| | GTAACCCATCTGTTGGAATATTCTACTTTGTTAGTTATATCATCATATC | |
| | CTTCCTGGTTGTGGTGAACATGTACATTGCAGTCATACTGGAGAATTT | |
| | TAGTGTTGCCACTGAAGAAAGTACTGAACCTCTGAGTGAGGATGACTT | |
| | TGAGATGTTCTATGAGGTTTGGGAGAAGTTTGATCCCGATGCGACCCA | |
| | GTTTATAGAGTTCTCTAAACTCTCTGATTTTGCAGCTGCCCTGGATCCT | |
| | CCTCTTCTCATAGCAAAACCCAACAAAGTCCAGCTCATTGCCATGGATC | |
| | TGCCCATGGTTAGTGGTGACCGGATCCATTGTCTTGACATCTTATTTG | |
| | TTTTTACAAAGCGT6TTT!GGGTGAGAGTGGGGAGATGGATTCTCTTC | |
| | GTTCACAGATGGAAGAAAGGTTCATGTCTGCAAATCCTTCCAAAGTGT | |
| | CCTATGAACCCATCACAACCACACTAAAACGGAAACAAGAGGATGTGT | |
| | CTGCTACTGTCATTCAGCGTGCTTATAGACGTTACCGCTTAAGGCAAA | |
| | ATGTCAAAAATATATCAAGTATATACATAAAAGATGGAGACAGAGATG | |
| | ATGATTTACTCAATAAAAAAGATATGGTTTTTGATAATGTTAATGAGAA | |
| | CTCAAGTCCAGAAAAAACAGATGCCACTTCATCCACCACCTCTCCACCT | |
| | TCATATGATAGTGTAACAAAGCCAGACAAAGAGAAATATGAACAAGAC | |
| | AGAACAGAAAAGGAAGACAAAGGGAAAGACAGCAAGGAAAGCAAAAAA | |
| | TAG | |
| 330 | ACCTGGGAAACATCCGCGAGCACAGCCTACATGGACCTGAGTAGTCTG | 22D04-Heavy chain |
| | AGATCGGAAGACACGGCTGTTTATTACTGTGCGAGAGGGTTTACTATG | nucleotide sequence |
| | GTTCGGGGAGCCCCCTATTATGACGGTATGGACGTCTGGGGCCAAGG | |
| | GACCACGGTCACCGTCTCCTCAG | |
| 331 | QVQTVQSGAEVRKPGASVTNSCKASGYTFTDYATHTNVRQAPGQRTEWM | 22D04-Heavy chain |
| | GWTTVGNGKTRYSQKFQGRVTTTVVETSASTAYMDTSSTRSEDTAVYYCA | amino acid sequence |
| | RGFTMVRGAPYYDGMDVWGQGTTVTVSS | |
| 332 | GATATTGTGATGACTCAGTCTCCACTCTCCCTGCCCGTCACCCCTGGA | 22D04-Light chain |
| | GAGCCGGCCTCCATCTCCTGCAGGTCTAGTCAGAGCCTCCTGTATAGT | nucleotide sequence |
| | ACTGGATACAACTATTTGGATTGGTACCTGCAGAAGCCAGGGCTGTCT | |
| | CCACAGCTCCTGATCTATTTGGGTTCTAATCGGGCCTCCGGGGTCCCT | |
| | GACAGGTTCAGTGGCAGTGGATCAGGCACAGATTTTACACTGAAAATC | |
| | AGCAGAGTGGAGGCTGAGGATGTTGGGCATTATTACTGCATGCAAGC | |
| | TCTACAAACTCCCACTTTCGGCGGAGGGACCAAGGTGGAGATCAAAC | |
| 333 | DTVMTQSPTSTPVTPGEPASTSCRSSQSTTYSTGYNYTDVVYTQKPGTSPQ | 22D04-Light chain |
| | TTTYTGSNRASGVPDRFSGSGSGTDFTTKTSRVEAEDVGVYYCMQATQTP | amino acid sequence |
| | TFGGGTKVETK | |
| 334 | DYATH | 22D04-CDRH1 amino |
| | | acid sequence (Kabat) |
| 335 | GYTFTDYA | 22D04-CDRH1 amino |
| | | acid sequence (IMGT) |
| 336 | WTTVGNGKTRYSQKFQG | 22D04-CDRH2 amino |
| | | acid sequence (Kabat) |
| 337 | TTVGNGKT | 22D04-CDRH2 amino |
| | | acid sequence (IMGT) |
| 338 | GFTMVRGAPYYDGMDV | 22D04-CDRH3 amino |
| | | acid sequence (Kabat) |
| 339 | ARGFTMVRGAPYYDGMDV | 22D04-CDRH3 amino |
| | | acid sequence (IMGT) |
| 340 | RSSQSTTYSTGYNYTD | 22D04-CDRL1 amino |
| | | acid sequence (Kabat) |
| 341 | QSTTYSTGYNY | 22D04-CDRL1 amino |
| | | acid sequence (IMGT) |
| 342 | TGSNRAS | 22D04-CDRL2 amino |
| | | acid sequence (Kabat) |
| 343 | TGS | 22D04-CDRL2 amino |
| | | acid sequence (IMGT) |
| 344 | MQATQTPT | 22D04-CDRL3 amino |
| | | acid sequence (Kabat) |
| 345 | MQATQTPT | 22D04-CDRL3 amino |
| | | acid sequence (IMGT) |
| 346 | CAGGTCCAGCTTGTGCAGTCTGGGGCAGAGGTGAGGAAGCCTGGGGC | 22G08-Heavy chain |
| | CTCAGTGAAGGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGACCACAGTCTA | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCGTCTCCTCAG | |
| 347 | QVQTVQSGAEVRKPGASVTNSCKASGYTFTDYATHWVRQAPGQRTEWM | 22G08-Heavy chain |
| | GWTTVGNGKTRYAQKFQGRVTTTWETSATTVYMDTSSTRSEDTAVYYCA | amino acid sequence |
| | RGFTMVRGAPYYDGTDVWGQGTTVTVSS | |
| 348 | GATATTGTGATGACTCAGTCTCCACTCTCCCTGCCCGTCACCCCTGGA | 22G08-Light chain |
| | GAGCCGGCCTCCATCTCCTGCAGGTCTAGTCAGAGCCTCCTGTATAGT | nucleotide sequence |
| | ACTGGATACAACTATTTGGATTGGTACCTGCAGAAGCCAGGGCTGTCT | |
| | CCACAGCTCCTGATCTATTTGGGTTCTAATCGGGCCTCCGGGGTCCCT | |
| | GACAGGTTCAGTGGCAGTGGATCAGGCACAGAATTTACACTGAAAATC | |
| | AGCAGAGTGGAGGCTGAGGATGTTGGGGTTTATTACTGCATGCAAGC | |
| | TCTACAAACTCCCACTTTCGGCGGAGGGACCAAGGTGGAGATCAAAC | |
| 349 | DTVMTQSPTSTPVTPGEPASTSCRSSQSTTYSTGYNYTDVVYTQKPGTSPQ | 22G08-Light chain |
| | TTTYTGSNRASGVPDRFSGSGSGTDFTTKTSRVEAEDVGVYYCMQATQTP | amino acid sequence |
| | TFGGGTKVETK | |
| 350 | DYATH | 22G08-CDRH1 amino |
| | | acid sequence (Kabat) |
| 351 | GYTFTDYA | 22G08-CDRH1 amino |
| | | acid sequence (IMGT) |
| 352 | WTTVGNGKTRYAQKFQG | 22G08-CDRH2 amino |
| | | acid sequence (Kabat) |
| 353 | TTVGNGKT | 22G08-CDRH2 amino |
| | | acid sequence (IMGT) |
| 354 | GFTMVRGAPYYDGTDV | 22G08-CDRH3 amino |
| | | acid sequence (Kabat) |
| 355 | ARGFTMVRGAPYYDGTDV | 22G08-CDRH3 amino |
| | | acid sequence (IMGT) |
| 356 | RSSQSTTYSTGYNYTD | 22G08-CDRL1 amino |
| | | acid sequence (Kabat) |
| 357 | QSTTYSTGYNY | 22G08-CDRL1 amino |
| | | acid sequence (IMGT) |
| 358 | TGSNRAS | 22G08-CDRL2 amino |
| | | acid sequence (Kabat) |
| 359 | TGS | 22G08-CDRL2 amino |
| | | acid sequence (IMGT) |
| 360 | MQATQTPT | 22G08-CDRL3 amino |
| | | acid sequence (Kabat) |
| 361 | MQATQTPT | 22G08-CDRL3 amino |
| | | acid sequence (IMGT) |
| 362 | CAGGTCCAGCTTGTGCAGTCTGGGGCTGAGGTGAGGAAGCCTGGGGC | 22G09-Heavy chain |
| | CTCAGTGAAAGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATTCACAGAAGTT | |
| | TCAGGGCAGAGTCACCATTACCTGGGAAACATCCGCGACCACAGCCTA | |
| | CATGGACCTGAGTAGTCTGAGATCGGAGGACACGGCTGTTTATTACTG | |
| | TGCGAGAGGGTTTACTATGGTTCGGGGAGCCCCCTATTATGACGGTAT | |
| | GGACGTCTGGGGCCAAGGGACCACGGTCACCGTCTCCTCAG | |
| 363 | QVQTVQSGAEVRKPGASVTNSCKASGYTFTDYATHWVRQAPGQRTEWM | 22G09-Heavy chain |
| | GWTTVGNGKTRYSQKFQGRVTFTWETSATTAYMDTSSTRSEDTAVYYCA | amino acid sequence |
| | RGFTMVRGAPYYDGMDVWGQGTTVTVSS | |
| 364 | GATATTGTGATGACTCAGTCTCCACTCTCCCTGCCCGTCACCCCTGGA | 22G09-Light chain |
| | GAGCCGGCCTCCATCTCCTGCAGGTCTAGTCAGAGCCTCCTGTATACT | nucleotide sequence |
| | ACTGGATACAACTATTTGGATTGGTACCTGCAGAAGCCAGGGCTGTCT | |
| | CCACAGCTCCTGATCTATTTGGGTTCTTATCGGGCCTCCGGGGTCCCT | |
| | GACAGGTTCACTGGCAGTGGATCAGGCACAGATTTTACACTGAAGATC | |
| | AGCAGAGTGGAGGCTGAGGATGTTGGGGTTTATTACTGCATGCAAGC | |
| | TCTACAAGCTCCTACTTTCGGCGGAGGGACCAAGTTGGAGATCAAAC | |
| 365 | DTVMTQSPTSTPVTPGEPASTSCRSSQSTTYTTGYNYTDVVYTQKPGTSPQ | 22G09-Light chain |
| | TTTYTGSYRASGVPDRFTGSGSGTDFTTKTSRVEAEDVGVYYCMQATQAP | amino acid sequence |
| | TFGGGTKTETK | |
| 366 | DYATH | 22G09-CDRH1 amino |
| | | acid sequence (Kabat) |
| 367 | GYTFTDYA | 22G09-CDRH1 amino |
| | | acid sequence (IMGT) |
| 368 | WTTVGNGKTRYSQKFQG | 22G09-CDRH2 amino |
| | | acid sequence (Kabat) |
| 369 | TTVGNGKT | 22G09-CDRH2 amino |
| | | acid sequence (IMGT) |
| 370 | GFTMVRGAPYYDGMDV | 22G09-CDRH3 amino |
| | | acid sequence (Kabat) |
| 371 | ARGFTMVRGAPYYDGMDV | 22G09-CDRH3 amino |
| | | acid sequence (IMGT) |
| 372 | RSSQSTTYTTGYNYTD | 22G09-CDRL1 amino |
| | | acid sequence (Kabat) |
| 373 | QSTTYTTGYNY | 22G09-CDRL1 amino |
| | | acid sequence (IMGT) |
| 374 | TGSYRAS | 22G09-CDRL2 amino |
| | | acid sequence (Kabat) |
| 375 | TGS | 22G09-CDRL2 amino |
| | | acid sequence (IMGT) |
| 376 | MQATQAPT | 22G09-CDRL3 amino |
| | | acid sequence (Kabat) |
| 377 | MQATQAPT | 22G09-CDRL3 amino |
| | | acid sequence (IMGT) |
| 378 | CAGGTTCAATTGGTGCAATCTGAAGCTGAGGTGAAGAAGCCCGGGGC | 25A01-Heavy chain |
| | CTCAGTGAAGGTCTCCTGCAAGGCTTCTGGTTATATCTTTACCACCTTT | nucleotide sequence |
| | GGTCTCAGCTGGGTGCGACAGGCCCCTGGACAAGGACTTGAGTGGAT | |
| | GGGAAGGATCACCACAAATAATGGGAACACAATCTATGAGAGGAGATT | |
| | CCAGGGCAGAGTCACCATGACCATAGACACATCCACGACAACTGCCTA | |
| | CATGGAGATGAGAAGCCTGACATCTGACGATACGGCCGTTTATTATTG | |
| | TGCGAGAGATGGGGCCCCACAGGACCACTGGGGCCAGGGAACCCTGG | |
| | TCACCGTCTCCTCAG | |
| 379 | QVQTVQSEAEVKKPGASVTNSCKASGYTFTTFGTSWVRQAPGQGTEWM | 25A01-Heavy chain |
| | GRTTTNNGNTTYERRFQGRVTMTTDTSTTTAYMEMRSTTSDDTAVYYCA | amino acid sequence |
| | RDGAPQDHWGQGTTVTVSS | |
| 380 | GACATCCAGATGACCCAGTCTCCATCCTCACTGTCTGTCTCTGTGGGA | 25A01-Light chain |
| | GACAGAGTCATCATCACCTGTCGGGCGAGTCAAGACATTAGAAATTCT | nucleotide sequence |
| | TTAGCCTGGTTTCAGCAAAAACCTGGGAAAGCCCCTAAGTCCCTGATC | |
| | TTTGCTGCATCCAGTTTGCAAAGTGGGGTCCCATCAAAGTTCCGCGGC | |
| | AGTGGATTTGGGACAGATTTCACTCTCACCATCACCAGCCTGCAGCCT | |
| | GAGGATTTTGCAACTTATTACTGCCAACAGTATAGTAGTTATCCTCTCA | |
| | CTTTTGGCGGAGGGACCAAGGTAGAGATCAAAC | |
| 381 | DTQMTQSPSSTSVSVGDRVTTTCRASQDTRNSTAWFQQKPGKAPKSTTFA | 25A01-Light chain |
| | ASSTQSGVPSKFRGSGFGTDFTTTTTSTQPEDFATYYCQQYSSYPTTFGG | amino acid sequence |
| | GTKVETK | |
| 382 | TFGTS | 25A01-CDRH1 amino |
| | | acid sequence (Kabat) |
| 383 | GYTFTTFG | 25A01-CDRH1 amino |
| | | acid sequence (IMGT) |
| 384 | RTTTNNGNTTYERRFQG | 25A01-CDRH2 amino |
| | | acid sequence (Kabat) |
| 385 | TTTNNGNT | 25A01-CDRH2 amino |
| | | acid sequence (IMGT) |
| 386 | DGAPQDH | 25A01-CDRH3 amino |
| | | acid sequence (Kabat) |
| 387 | ARDGAPQDH | 25A01-CDRH3 amino |
| | | acid sequence (IMGT) |
| 388 | RASQDTRNSTA | 25A01-CDRL1 amino |
| | | acid sequence (Kabat) |
| 389 | QDTRNS | 25A01-CDRL1 amino |
| | | acid sequence (IMGT) |
| 390 | AASSTQS | 25A01-CDRL2 amino |
| | | acid sequence (Kabat) |
| 391 | AAS | 25A01-CDRL2 amino |
| | | acid sequence (IMGT) |
| 392 | QQYSSYPTT | 25A01-CDRL3 amino |
| | | acid sequence (Kabat) |
| 393 | QQYSSYPTT | 25A01-CDRL3 amino |
| | | acid sequence (IMGT) |
| 394 | GAGGTGCACTTGGTAGAATCTGGGGGAGGCTTGGTTCAGCCGGGGGG | 25C01-Heavy chain |
| | GTCCCTGAGACTCTCCTGTTCAGCCTCTAGATTCACCTTTAGCACCTCT | nucleotide sequence |
| | GCCATGACCTGGGTCCGCCAGGCTCCAGGGAAGGGGCTGGAGTGGGT | |
| | CTCAGTTATTAGTGCTAGTGGTACTACCACATATTACGGAGACTCCGT | |
| | GAAGGGCCGGTTCACCATCTCCAGAGACAATTCCAAGAACACGCTACA | |
| | TCTGCAAATGAACAGCCTGAGAGCCGAGGACACGGCCGTATATTATTG | |
| | TGCGAAAGGGGGTTTGGCAGTGCCTGGTCCGAACTACTGGGGCCAGG | |
| | GAACCCTGGTCACCGTCTCCTCAG | |
| 395 | EVHTVESGGGTVQPGGSTRTSCSASRFTFSTSAMTWVRQAPGKGTEWVS | 25C01-Heavy chain |
| | VTSASGTTTYYGDSVKGRFTTSRDNSKNTTHTQMNSTRAEDTAVYYCAKG | amino acid sequence |
| | GTAVPGPNYWGQGTTVTVSS | |
| 396 | GATGTTGTGATGACTCAGTCTCCACTCTCCCTGCCCGTCACCCTTGGA | 25C01-Light chain |
| | CAGCCGGCCTCCATCTCCTGCAGGTCCAGTCAAAGCCTCGTCTTCAGT | nucleotide sequence |
| | GATGGAAACACCTACTTGACTTGGTTTCAACAGAGGCCAGGCCAATCT | |
| | CCAAGGCGCCTAATTTATAAGGTTTCTGACCGGGACTCTGGGGTCCCA | |
| | GACAGATTCAGCGGCAGTGGGTCAGGCACTGATTTCACACTGCAAATC | |
| | AGCAGGGTGGAGGCTGAGGATGTTGGCCTTTATTACTGCATGCAAGG | |
| | TTCACACTGGCCGCTCACTTTCGGCGGAGGGACCAAGGTGGAGATCAA | |
| | AC | |
| 397 | DVVMTQSPTSTPVTTGQPASTSCRSSQSTVFSDGNTYTTTNFQQRPGQSP | 25C01-Light chain |
| | RRTTYTNSDRDSGVPDRFSGSGSGTDFTTQTSRVEAEDVGTYYCMQGSH | amino acid sequence |
| | WPTTFGGGTKVETK | |
| 398 | TSAMT | 25C01-CDRH1 amino |
| | | acid sequence (Kabat) |
| 399 | RFTFSTSA | 25C01-CDRH1 amino |
| | | acid sequence (IMGT) |
| 400 | VTSASGTTTYYGDSVKG | 25C01-CDRH2 amino |
| | | acid sequence (Kabat) |
| 401 | TSASGTTT | 25C01-CDRH2 amino |
| | | acid sequence (IMGT) |
| 402 | GGTAVPGPNY | 25C01-CDRH3 amino |
| | | acid sequence (Kabat) |
| 403 | AKGGTAVPGPNY | 25C01-CDRH3 amino |
| | | acid sequence (IMGT) |
| 404 | RSSQSTVFSDGNTYTT | 25C01-CDRL1 amino |
| | | acid sequence (Kabat) |
| 405 | QSTVFSDGNTY | 25C01-CDRL1 amino |
| | | acid sequence (IMGT) |
| 406 | KVSDRDS | 25C01-CDRL2 amino |
| | | acid sequence (Kabat) |
| 407 | KVS | 25C01-CDRL2 amino |
| | | acid sequence (IMGT) |
| 408 | MQGSHWPTT | 25C01-CDRL3 amino |
| | | acid sequence (Kabat) |
| 409 | MQGSHWPTT | 25C01-CDRL3 amino |
| | | acid sequence (IMGT) |
| 410 | CAGGTTCAGTTGGTACAGTCTGGACCTGAAGTGAAGAAGCCTGGGGC | 25F08-Heavy chain |
| | CTCAGTGAAGGTCTCCTGCCAGGCTTCTGGTTATACCTTTACCACCTA | nucleotide sequence |
| | TGGCATCAACTGGGTGCGACAGGCCCCTGGACAAGGACTTGAGTGGA | |
| | TGGGAAGGATCAGCGCTTACAATGGTAACACAAATTATGCACAGAAGT | |
| | TCCAGGGCAGAGTCACCATGACCACAGACACATCTACGAGGACAGCCT | |
| | ACATGGAGATGAGTAACCTGATATCTGACGACACGGCCGTGTATTATT | |
| | GTGCGCGAGATGGGGCTCCCCAAGACCACTGGGGCCAGGGAACCCTA | |
| | ATCACCGTCTCTTCAG | |
| 411 | QVQTVQSGPEVKKPGASVTNSCQASGYTFTTYGTNVVVRQAPGQGTEWM | 25F08-Heavy chain |
| | GRTSAYNGNTNYAQKFQGRVTMTTDTSTRTAYMEMSNTTSDDTAVYYC | amino acid sequence |
| | ARDGAPQDHWGQGTTTTVSS | |
| 412 | GACATCCAGATGACCCAGTCTCCATCCTCACTGTCTGTATCTGTAGGA | 25F08-Light chain |
| | GACAGAATCACCATCACCTGTCGGGCGAGTCAGGACATTAGTAATTCT | nucleotide sequence |
| | TTAGCCTGGTTTCAGCAGAAACCAGGGAAAGCCCCTAAGTCCCTGATC | |
| | TTTGCTGCATCCAGTTTGCAAAGTGGGGTCCCATCAAAGTTCAGCGGC | |
| | AGTGGATCTGGGACAGATTTCAATTTTACCATCAGCAGCCTGCAGCCT | |
| | GAAGATTTGCAACTTATTACTGCCAACAGTATAATAGTTTCCCTCTCA | |
| | CTTTCGGCGGAGGGACCAAGGTGGAGATCAAAC | |
| 413 | DTQMTQSPSSTSVSVGDRTTTTCRASQDTSNSTAWFQQKPGKAPKSTTFA | 25F08-Light chain |
| | ASSTQSGVPSKFSGSGSGTDFNFTTSSTQPEDFATYYCQQYNSFPTTFGG | amino acid sequence |
| | GTKVETK | |
| 414 | TYGTN | 25F08-CDRH1 amino |
| | | acid sequence (Kabat) |
| 415 | GYTFTTYG | 25F08-CDRH1 amino |
| | | acid sequence (IMGT) |
| 416 | RTSAYNGNTNYAQKFQG | 25F08-CDRH2 amino |
| | | acid sequence (Kabat) |
| 417 | TSAYNGNT | 25F08-CDRH2 amino |
| | | acid sequence (IMGT) |
| 418 | DGAPQDH | 25F08-CDRH3 amino |
| | | acid sequence (Kabat) |
| 419 | ARDGAPQDH | 25F08-CDRH3 amino |
| | | acid sequence (IMGT) |
| 420 | RASQDTSNSTA | 25F08-CDRL1 amino |
| | | acid sequence (Kabat) |
| 421 | QDTSNS | 25F08-CDRL1 amino |
| | | acid sequence (IMGT) |
| 422 | AASSTQS | 25F08-CDRL2 amino |
| | | acid sequence (Kabat) |
| 423 | MS | 25F08-CDRL2 amino |
| | | acid sequence (IMGT) |
| 424 | QQYNSFPTT | 25F08-CDRL3 amino |
| | | acid sequence (Kabat) |
| 425 | QQYNSFPTT | 25F08-CDRL3 amino |
| | | acid sequence (IMGT) |
| 426 | GAGGTGCAGCTGGTGGAGTCTGGGGGAGGCCTGGTCAAGCCTGGGG | 27A03-Heavy chain |
| | GGTCCCTGAGACTCTCCTGTGCAGCCTCTGGATTCACCTTCAGTACCT | nucleotide sequence |
| | ATAGCATGAACTGGGTCCGCCAGGCTCCAGGGAAGGGGCTGGAGTGG | |
| | GTCTCATCCATTAGTCGTAGTAGTAGTTACATATACTACGCAGACTCA | |
| | GTGAAGGGCCGATTCACCATCTCCAGAGACAACGCCAAGAACTCACTG | |
| | TCTCTGCAAATGAACAGCCTGAGAGCCGAGGACACAGCTGTGTATTAC | |
| | TGTGCGAGAGGAGCAGCAGCTGGTACGGACTACTGGGGCCAGGGAAC | |
| | CCTGGTCACCGTCTCCTCAG | |
| 427 | EVQTVESGGGTVKPGGSTRTSCAASGFTFSTYSMNTNVRQAPGKGTEWV | 27A03-Heavy chain |
| | SSTSRSSSYTYYADSVKGRFTTSRDNAKNSTSTQMNSTRAEDTAVYYCAR | amino acid sequence |
| | GAAAGTDYWGQGTTVTVSS | |
| 428 | GATATTGTGATGACCCAGACTCCACTCTCCTCACCTGTCACCCTTGGAC | 27A03-Light chain |
| | AGCCGGCCTCCATCTCCTGCAGGTCTAGTCAAAGCCTCGTACACAGTG | nucleotide sequence |
| | ATGGAAACACCTACTTGAGTTGGCTTCAGCAGAGGCCAGGCCAGCCTC | |
| | CAAGACTCCTAATTTATAGGATTTCTAACCGGTTCTCTGGGGTCCCAG | |
| | ACAGATTCAGTGGCAGTGGGGCAGGGACAGATTTCACACTGAAAATCA | |
| | GCAGGGTGGAAGCTGAGGATGTCGGGGTTTATTACTGCATGCAAACT | |
| | ACACAATTTCCGCTCACTTTCGGCGGAGGGACCAAGGTGGAGATCAAA | |
| | C | |
| 429 | DTVMTQTPTSSPVTTGQPASTSCRSSQSTVHSDGNTYTSWTQQRPGQPP | 27A03-Light chain |
| | RTTTYRTSNRFSGVPDRFSGSGAGTDFTTKTSRVEAEDVGVYYCMQTTQF | amino acid sequence |
| | PTTFGGGTKVETK | |
| 430 | TYSMN | 27A03-CDRH1 amino |
| | | acid sequence (Kabat) |
| 431 | GFTFSTYS | 27A03-CDRH1 amino |
| | | acid sequence (IMGT) |
| 432 | STSRSSSYTYYADSVKG | 27A03-CDRH2 amino |
| | | acid sequence (Kabat) |
| 433 | TSRSSSYT | 27A03-CDRH2 amino |
| | | acid sequence (IMGT) |
| 434 | GAAAGTDY | 27A03-CDRH3 amino |
| | | acid sequence (Kabat) |
| 435 | ARGAAAGTDY | 27A03-CDRH3 amino |
| | | acid sequence (IMGT) |
| 436 | RSSQSTVHSDGNTYTS | 27A03-CDRL1 amino |
| | | acid sequence (Kabat) |
| 437 | QSTVHSDGNTY | 27A03-CDRL1 amino |
| | | acid sequence (IMGT) |
| 438 | RTSNRFS | 27A03-CDRL2 amino |
| | | acid sequence (Kabat) |
| 439 | RTS | 27A03-CDRL2 amino |
| | | acid sequence (IMGT) |
| 440 | MQTTQFPTT | 27A03-CDRL3 amino |
| | | acid sequence (Kabat) |
| 441 | MQTTQFPTT | 27A03-CDRL3 amino |
| | | acid sequence (IMGT) |
| 442 | CAGGTCCAGCTTGTGCAGTCTGGGGCTGAGGTGAGGAAGCCTGGGGC | 28608-Heavy chain |
| | CTCAGTGAAGGTTTCCTGCAAGGCTTCTGGATACACCTTCACTGACTA | nucleotide sequence |
| | TGCACTACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGAGCACAGCCTT | |
| | CGTGGACCTGAATAGTCTGAGATCGGAAGACACGGCTTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCGTCTCCTCAG | |
| 443 | QVQTVQSGAEVRKPGASVKVSCKASGYTFTDYATHTANRQAPGQRTEWM | 28608-Heavy chain |
| | GWTTVGNGKTRYAQKFQGRVTTTWETSASTAFVDTNSTRSEDTAVYYCA | amino acid sequence |
| | RGFTMVRGAPYYDGTDVWGQGTTVTVSS | |
| 444 | GATATTGTGATGACTCAGTCTCCACTCTCCCTGTCCGTCACCCCTGGA | 28608-Light chain |
| | GAGCCGGCCTCCATCTCCTGCAGGTCTAGTCAGAGCCTCCTGTATAGT | nucleotide sequence |
| | ACTGGATACAACTATTTGGATTGGTACCTGCAGAAGCCAGGGCTGTCT | |
| | CCACAGCTCCTGATCTATTTGGGTTCTAATCGGGCCTCCGGGGTCCCT | |
| | GACAGGTTCAGTGGCAGTGGATCAGGCACAGATTTACACTGAAGATC | |
| | AGCAGAGTGGAGGCTGAAGATGTTGGGTTTTATTACTGCATGCAAGCT | |
| | CTACAAGCTCCCACTTTCGGCGGAGGGACCAAGGTGGAGATCAAAC | |
| 445 | DTVMTQSPTSTSVTPGEPASTSCRSSQSTTYSTGYNYTDVVYTQKPGTSPQ | 281308-Light chain |
| | TTTYTGSNRASGVPDRFSGSGSGTDFTTKTSRVEAEDVGFYYCMQATQAP | amino acid sequence |
| | TFGGGTTNETK | |
| 446 | DYATH | 28608-CDRH1 amino |
| | | acid sequence (Kabat) |
| 447 | GYTFTDYA | 28608-CDRH1 amino |
| | | acid sequence (IMGT) |
| 448 | WTTVGNGKTRYAQKFQG | 28608-CDRH2 amino |
| | | acid sequence (Kabat) |
| 449 | TTVGNGKT | 28608-CDRH2 amino |
| | | acid sequence (IMGT) |
| 450 | GFTMVRGAPYYDGTDV | 28608-CDRH3 amino |
| | | acid sequence (Kabat) |
| 451 | ARGFTMVRGAPYYDGTDV | 28608-CDRH3 amino |
| | | acid sequence (IMGT) |
| 452 | RSSQSTTYSTGYNYTD | 28608-CDRL1 amino |
| | | acid sequence (Kabat) |
| 453 | QSTTYSTGYNY | 28608-CDRL1 amino |
| | | acid sequence (IMGT) |
| 454 | TGSNRAS | 28608-CDRL2 amino |
| | | acid sequence (Kabat) |
| 455 | TGS | 28608-CDRL2 amino |
| | | acid sequence (IMGT) |
| 456 | MQATQAPT | 28608-CDRL3 amino |
| | | acid sequence (Kabat) |
| 457 | MQATQAPT | 281308-CDRL3 amino |
| | | acid sequence (IMGT) |
| 458 | CAGGTCCAACTTGTGCAGTCTGGGGCTGAGGTGAGGAAGCCTGGGGC | 28C11-Heavy chain |
| | CTCAGTGAAGGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGAGCACAGCCTA | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAGGGGACCACGGTCACCGTCTCCTCAG | |
| 459 | QVQTVQSGAEVRKPGASVTNSCKASGYTFTDYATHVVVRQAPGQRTEWM | 28C11-Heavy chain |
| | GWTTVGNGKTRYAQKFQGRVTTTVVETSASTAYMDTSSTRSEDTAVYYCA | amino acid sequence |
| | RGFTMVRGAPYYDGTDVWGQGTTVTVSS | |
| 460 | GATATTGTGATGACTCAGTCTCCACTCTCCCTGCCCGTCACCCCTGGA | 28C11-Light chain |
| | GAGCCGGCCTCCATCTCCTGCAGGTCTAGTCAGAGCCTCCTGTTTAGT | nucleotide sequence |
| | ACTGGATACAACTATTTGGATTGGTACCTGCAGAAGCCAGGGCTGTCT | |
| | CCACAGCTCCTGATCTATTTGGGTTCTAATCGGGCCTCCGGGGTCCCT | |
| | GACAGGTTCAGTGGCAGTGGATCAGGCACAGATTACACTGAAAATC | |
| | AGCAGAGTGGAGGCTGAGGATGTTGGGGTTTATTACTGCATGCAAGC | |
| | TCTACAAACTCCCACTTTCGGCGGAGGGACCAAGGTGGAGATCAAAC | |
| 461 | DTVMTQSPTSTPVTPGEPASTSCRSSQSTTFSTGYNYTDWYTQKPGTSPQT | 28C11-Light chain |
| | TTYTGSNRASGVPDRFSGSGSGTDFTTKTSRVEAEDVGVYYCMQATQTPT | amino acid sequence |
| | FGGGTKVETK | |
| 462 | DYATH | 28C11-CDRH1 amino |
| | | acid sequence (Kabat) |
| 463 | GYTFTDYA | 28C11-CDRH1 amino |
| | | acid sequence (IMGT) |
| 464 | WTTVGNGKTRYAQKFQG | 28C11-CDRH2 amino |
| | | acid sequence (Kabat) |
| 465 | TTVGNGKT | 28C11-CDRH2 amino |
| | | acid sequence (IMGT) |
| 466 | GFTMVRGAPYYDGTDV | 28C11-CDRH3 amino |
| | | acid sequence (Kabat) |
| 467 | ARGFTMVRGAPYYDGTDV | 28C11-CDRH3 amino |
| | | acid sequence (IMGT) |
| 468 | RSSQSTTFSTGYNYTD | 28C11-CDRL1 amino |
| | | acid sequence (Kabat) |
| 469 | QSTTFSTGYNY | 28C11-CDRL1 amino |
| | | acid sequence (IMGT) |
| 470 | TGSNRAS | 28C11-CDRL2 amino |
| | | acid sequence (Kabat) |
| 471 | TGS | 28C11-CDRL2 amino |
| | | acid sequence (IMGT) |
| 472 | MQATQTPT | 28C11-CDRL3 amino |
| | | acid sequence (Kabat) |
| 473 | MQATQTPT | 28C11-CDRL3 amino |
| | | acid sequence (IMGT) |
| 474 | CAGGTGCAGCTGCAGGAGTCGGGCCCAGGACTGGTGAAGCCTTCGGG | 30G01-Heavy chain |
| | GACCCTGTCCCTCACTTGCGCTGTCTCTGGTGGCTCCATCAGTAGTAG | nucleotide sequence |
| | TAACTGGTGGAGTTGGGTCCGCCAGCCCCCAGGGAAGGGGCTGGAGT | |
| | GGATTGGGGAAATCTATCATAGTGGGAACACCAACTACAACCCGTCCC | |
| | TCAAGAGTCGAGTCACCATATCAGTAGACAAGTCCAAGAACCAGTTCT | |
| | CCCTGAAGCTGAGCTCTGTGACCGCCGCGGACACGGCCGTGTATTACT | |
| | GTGTGAGAGGTTATTACTATGATTCGGGGACCTCTTGGGGGTACTATT | |
| | ATGGTATGGACGTCTGGGGCCAAGGGACCACGGTCACCGTCTCCTCAG | |
| 475 | QVQTQESGPGTVKPSGTTSTTCAVSGGSTSSSNWWSWVRQPPGKGTEW | 30G01-Heavy chain |
| | TGETYHSGNTNYNPSTKSRVTTSVDKSKNQFSTKTSSVTAADTAVYYCVRG | amino acid sequence |
| | YYYDSGTSWGYYYGMDVWGQGTTVTVSS | |
| 476 | GACATCCAGATGACCCAGTCTCCATCCTCACTGTCTGCATCTGTAGGA | 30G01-Light chain |
| | GACAGAGTCACCATCACTTGTCGGGCGAGTCAGGGCATTAGCAATTAT | nucleotide sequence |
| | TTAGCCTGGTTTCAGCAGAGACCAGGGAGAGCCCCTAAGTCCUTATC | |
| | TATGCTGCATCCAGTTTGCAAAGGGGGGTCCCATCAAAGTTCAGCGGC | |
| | AGTGGATCTGGGACAGATTTCACTCTCACCATCAGCAGCCTGCAGCCT | |
| | GAAGATTTGCAACTTATTACTGCCAACAGTATAATAGTTACCCGATCA | |
| | CCTTCGGCCAAGGGACACpACTGGAGATCAAAC | |
| 477 | DTQMTQSPSSTSASVGDRVTTTCRASQGTSNYTAWFQQRPGRAPKSTTYA | 30G01-Light chain |
| | ASSTQRGVPSKFSGSGSGTDFTTTTSSTQPEDFATYYCQQYNSYPTTFGQ | amino acid sequence |
| | GTRTETK | |
| 478 | SSNWWS | 30G01-CDRH1 amino |
| | | acid sequence (Kabat) |
| 479 | GGSTSSSNW | 30G01-CDRH1 amino |
| | | acid sequence (IMGT) |
| 480 | ETYHSGNTNYNPSTKS | 30G01-CDRH2 amino |
| | | acid sequence (Kabat) |
| 481 | TYHSGNT | 30G01-CDRH2 amino |
| | | acid sequence (IMGT) |
| 482 | GYYYDSGTSWGYYYGMDV | 30G01-CDRH3 amino |
| | | acid sequence (Kabat) |
| 483 | VRGYYYDSGTSWGYYYGMDV | 30G01-CDRH3 amino |
| | | acid sequence (IMGT) |
| 484 | RASQGTSNYTA | 30G01-CDRL1 amino |
| | | acid sequence (Kabat) |
| 485 | QGTSNY | 30G01-CDRL1 amino |
| | | acid sequence (IMGT) |
| 486 | AASSTQR | 30G01-CDRL2 amino |
| | | acid sequence (Kabat) |
| 487 | AAS | 30G01-CDRL2 amino |
| | | acid sequence (IMGT) |
| 488 | QQYNSYPTT | 30G01-CDRL3 amino |
| | | acid sequence (Kabat) |
| 489 | QQYNSYPTT | 30G01-CDRL3 amino |
| | | acid sequence (IMGT) |
| 490 | CAGGTCCAGCTTGTGCAGTCTGGGCCTGAGGTGAGGAACCCTGGGGC | 32A07-Heavy chain |
| | CTCAGTGAGGGTTTCCTGCAAGGCTTCTGGATACACCTTCACTGACTA | nucleotide sequence |
| | TGCACTACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGAGCACAGCCTT | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCATCTCCTCAG | |
| 491 | QVQTVQSGPEVRNPGASVRVSCKASGYTFTDYATHWVRQAPGQRTEW | 32A07-Heavy chain |
| | MGWTTVGNGKTRYAQKFQGRVTMNETSASTAFMDTSSTRSEDTAVYYC | amino acid sequence |
| | ARGFTMVRGAPYYDGTDVTNGQGTTVTTSS | |
| 492 | GATATTGTAATGACTCAGTCTCCACTCTCCCTGCCCGTCACCCTTGGAC | 32A07-Light chain |
| | AGCCGGCCTCCATCTCCTGCAGGTCTAGTCAAAGCCTCATATACAGTG | nucleotide sequence |
| | ATGGAAACACCTACTTGAATTGGTTTCAGCAGAGGCCAGGCCAATCTC | |
| | CAAGGCGCCTAATTTATAAGGTTTCTAACCGGGACTCTGGGGTCCCAG | |
| | ACAGATTCAGCGGCAGTGGGTCAGGCACTGATTTCACACTGAAAATCA | |
| | GCAGGGTGGAGGCTGAAGATGTTGGAGTTTATTACTGCTTGCAAGGT | |
| | ACTCTCTGGCCGATCACCTTCGGCCAAGGGACACGACTGGAGATCAAA | |
| | C | |
| 493 | DTVMTQSPTSTPVTTGQPASTSCRSSQSTTYSDGNTYTNWFQQRPGQSPR | 32A07-Light chain |
| | RTTYTNSNRDSGVPDRFSGSGSGTDFTTKTSRVEAEDVGVYYCTQGTTWP | amino acid sequence |
| | TTFGQGTRTETK | |
| 494 | DYATH | 32A07-CDRH1 amino |
| | | acid sequence (Kabat) |
| 495 | GYTFTDYA | 32A07-CDRH1 amino |
| | | acid sequence (IMGT) |
| 496 | WTTVGNGKTRYAQKFQG | 32A07-CDRH2 amino |
| | | acid sequence (Kabat) |
| 497 | TTVGNGKT | 32A07-CDRH2 amino |
| | | acid sequence (IMGT) |
| 498 | GFTMVRGAPYYDGTDV | 32A07-CDRH3 amino |
| | | acid sequence (Kabat) |
| 499 | ARGFTMVRGAPYYDGTDV | 32A07-CDRH3 amino |
| | | acid sequence (IMGT) |
| 500 | RSSQSTTYSDGNTYTN | 32A07-CDRL1 amino |
| | | acid sequence (Kabat) |
| 501 | QSTTYSDGNTY | 32A07-CDRL1 amino |
| | | acid sequence (IMGT) |
| 502 | KVSNRDS | 32A07-CDRL2 amino |
| | | acid sequence (Kabat) |
| 503 | TNS | 32A07-CDRL2 amino |
| | | acid sequence (IMGT) |
| 504 | TQGTTWPTT | 32A07-CDRL3 amino |
| | | acid sequence (Kabat) |
| 505 | TQGTTWPTT | 32A07-CDRL3 amino |
| | | acid sequence (IMGT) |
| 506 | CAGGTCCAGTTTGTGCAGTCTGGGGCTGAGGTGAGGAAGCCTGGGGC | 321304-Heavy chain |
| | CTCAGTGAAAGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATTCACAGAAGTT | |
| | TCAGGGCAGACTCACCATTACCTGGGAAACATCCGCGACCACAGCCTA | |
| | CATGGACCTGAGTAGTCTGAGATCGGAGGACACGGCTGTTTATTACTG | |
| | TGCGAGAGGGTTTACTATGGTTCGGGGAGCCCCCTATTATGACGGTAT | |
| | GGACGTCTGGGGCCAAGGGACCACGGTCACCGTCTCCTCAG | |
| 507 | QVQFVQSGAEVRKPGASVKVSCKASGYTFTDYATHVVVRQAPGQRTEWM | 32604-Heavy chain |
| | GWTTVGNGKTRYSQKFQGRTTTTVVETSATTAYMDTSSTRSEDTAVYYCA | amino acid sequence |
| | RGFTMVRGAPYYDGMDVWGQGTTVTVSS | |
| 508 | GATATTGTGATGACTCAGTCTCCACTCTCCCTGCCCGTCACCCCTGGA | 32604-Light chain |
| | GAGCCGGCCTCCATCTCCTGCAGGTCTAGTCAGAGCCTCCTGTATACT | nucleotide sequence |
| | ACTGGATACAACTATTTGGATTGGTACCTGCAGAAGCCAGGGCTGTCT | |
| | CCACAGCTCCTGATCTATTTGGGTTCTTATCGGGCCTCCGGGGTCCCT | |
| | GACAGGTTCACTGGCAGTGGATCAGGCACAGATTTACACTGAAGATC | |
| | AGCAGAGTGGAGGCTGAGGATGTTGGGGTTTATTACTGCATGCAAGC | |
| | TCTACAAGCTCCTACTTTCGGCGGAGGGACCAAGTTGGAGATCAAAC | |
| 509 | DTVMTQSPTSTPVTPGEPASTSCRSSQSTTYTTGYNYTDWYTQKPGTSPQ | 32604-Light chain |
| | TTTYTGSYRASGVPDRFTGSGSGTDFTTKTSRVEAEDVGVYYCMQATQAP | amino acid sequence |
| | TFGGGTKTETK | |
| 510 | DYATH | 32604-CDRH1 amino |
| | | acid sequence (Kabat) |
| 511 | GYTFTDYA | 32604-CDRH1 amino |
| | | acid sequence (IMGT) |
| 512 | WTTVGNGKTRYSQKFQG | 32604-CDRH2 amino |
| | | acid sequence (Kabat) |
| 513 | TTVGNGKT | 32604-CDRH2 amino |
| | | acid sequence (IMGT) |
| 514 | GFTMVRGAPYYDGMDV | 32604-CDRH3 amino |
| | | acid sequence (Kabat) |
| 515 | ARGFTMVRGAPYYDGMDV | 32604-CDRH3 amino |
| | | acid sequence (IMGT) |
| 516 | RSSQSTTYTTGYNYTD | 32604-CDRL1 amino |
| | | acid sequence (Kabat) |
| 517 | QSTTYTTGYNY | 32604-CDRL1 amino |
| | | acid sequence (IMGT) |
| 518 | TGSYRAS | 32604-CDRL2 amino |
| | | acid sequence (Kabat) |
| 519 | TGS | 32604-CDRL2 amino |
| | | acid sequence (IMGT) |
| 520 | MQATQAPT | 32604-CDRL3 amino |
| | | acid sequence (Kabat) |
| 521 | MQATQAPT | 32604-CDRL3 amino |
| | | acid sequence (IMGT) |
| 522 | CAGGTCCAGCTTGTGCAGTCTGGGCCTGAGGTGAGGAACCCTGGGGC | 32D04-Heavy chain |
| | CTCAGTGAGGGTTTCCTGCAAGGCTTCTGGATACACCTTCACTGACTA | nucleotide sequence |
| | TGCACTACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGAGCACAGCCTT | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCATCTCCTCAG | |
| 523 | QVQTVQSGPEVRNPGASVRVSCKASGYTFTDYATHVVVRQAPGQRTEW | 32D04-Heavy chain |
| | MGWTTVGNGKTRYAQKFQGRVTTTVVETSASTAFMDTSSTRSEDTAVYYC | amino acid sequence |
| | ARGFTMVRGAPYYDGTDVWGQGTTVTTSS | |
| 524 | GATATTGTGATGACTCAGTCTCCACTCTCCCTGCCCGTCAGCCCTGGA | 32D04-Light chain |
| | GAGCCGGCCTCCATCTCCTGCAGGTCTAGTCAGAGCCTCCTGTATAGC | nucleotide sequence |
| | ACTGGATACAACTATTTGGATTGGTACCTGCAGAAGCCAGGGCTGTCT | |
| | CCTCAGCTCCTGATCTATTTGGGTTCTATTCGGGCCTCCGGGGTCCCT | |
| | GACAGGTTCAGTGGCAGTGGATCAGGCACAGATTTTACACTGAGCATC | |
| | AGCAGAGTGGAGGCTGAGGATGTTGGAATTTATTACTGCATGCAAGCT | |
| | CTACAAACTCCCACTTTCGGCGGAGGGACCAAGGTGGAGATCAAAC | |
| 525 | DTVMTQSPTSTPVSPGEPASTSCRSSQSTTYSTGYNYTDVVYTQKPGTSPQ | 32D04-Light chain |
| | TTTYTGSTRASGVPDRFSGSGSGTDFTTSTSRVEAEDVGTYYCMQATQTPT | amino acid sequence |
| | FGGGTKVETK | |
| 526 | DYATH | 32D04-CDRH1 amino |
| | | acid sequence (Kabat) |
| 527 | GYTFTDYA | 32D04-CDRH1 amino |
| | | acid sequence (IMGT) |
| 528 | WTTVGNGKTRYAQKFQG | 32D04-CDRH2 amino |
| | | acid sequence (Kabat) |
| 529 | TTVGNGKT | 32D04-CDRH2 amino |
| | | acid sequence (IMGT) |
| 530 | GFTMVRGAPYYDGTDV | 32D04-CDRH3 amino |
| | | acid sequence (Kabat) |
| 531 | ARGFTMVRGAPYYDGTDV | 32D04-CDRH3 amino |
| | | acid sequence (IMGT) |
| 532 | RSSQSTTYSTGYNYTD | 32D04-CDRL1 amino |
| | | acid sequence (Kabat) |
| 533 | QSTTYSTGYNY | 32D04-CDRL1 amino |
| | | acid sequence (IMGT) |
| 534 | TGSTRAS | 32D04-CDRL2 amino |
| | | acid sequence (Kabat) |
| 535 | TGS | 32D04-CDRL2 amino |
| | | acid sequence (IMGT) |
| 536 | MQATQTPT | 32D04-CDRL3 amino |
| | | acid sequence (Kabat) |
| 537 | MQATQTPT | 32D04-CDRL3 amino |
| | | acid sequence (IMGT) |
| 538 | CAGGTCCAGCTTGTGCAGTCTGGGGCTGAGGTGAGGAAGCCTGGGGC | 32E01-Heavy chain |
| | CTCAGTGAAGGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATTCACAGAAGTT | |
| | TCAGGGCAGAGTCACCATTACCTGGGAAACATCCGCGAGCACAGCCTA | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTAT | |
| | GGACGTCTGGGGCCAAGGGACCACGGTCACCGTCTCCTCAG | |
| 539 | QVQTVQSGAEVRKPGASVKVSCKASGYTFTDYATHWVRQAPGQRTEWM | 32E01-Heavy chain |
| | GWTTVGNGKTRYSQKFQGRVTTTWETSASTAYMDTSSTRSEDTAVYYCA | amino acid sequence |
| | RGFTMVRGAPYYDGMDVWGQGTTVTVSS | |
| 540 | GATGTTGTAATGACTCAGTCTCCACTCTCCCTGCCCGTCACCCTTGGA | 32E01-Light chain |
| | CAGCCGGCCTCCATCTCCTGCAGGTCTAGTCAAAGCCTCGTATACAGT | nucleotide sequence |
| | GATGGAAACACCTACTTGAATTGGTTTCAGCAGAGGCCAGGCCAATCT | |
| | CCACGGCGCCTAATTTATAAGGTTTCTAACCGGGACTCTGGGGTCCCA | |
| | GACAGATTCAGCGGCAGTGGGTCAGGCACTGATTTCACACTGAAAATC | |
| | AGCAGGGTGGAGGCTGAAGATGTTGGAGTTTATTACTGCATGCAAGG | |
| | TACTCTCTGGCCGATCACCCTCGGCCAAGGGACACGACTGGAGATCAA | |
| | AC | |
| 541 | DVVMTQSPTSTPVTTGQPASTSCRSSQSTVYSDGNTYTNWFQQRPGQSP | 32E01-Light chain |
| | RRTTYTNSNRDSGVPDRFSGSGSGTDFTTKTSRVEAEDVGVYYCMQGTT | amino acid sequence |
| | WPTTTGQGTRTETK | |
| 542 | DYATH | 32E01-CDRH1 amino |
| | | acid sequence (Kabat) |
| 543 | GYTFTDYA | 32E01-CDRH1 amino |
| | | acid sequence (IMGT) |
| 544 | WTTVGNGKTRYSQKFQG | 32E01-CDRH2 amino |
| | | acid sequence (Kabat) |
| 545 | TTVGNGKT | 32E01-CDRH2 amino |
| | | acid sequence (IMGT) |
| 546 | GFTMVRGAPYYDGMDV | 32E01-CDRH3 amino |
| | | acid sequence (Kabat) |
| 547 | ARGFTMVRGAPYYDGMDV | 32E01-CDRH3 amino |
| | | acid sequence (IMGT) |
| 548 | RSSQSTVYSDGNTYTN | 32E01-CDRL1 amino |
| | | acid sequence (Kabat) |
| 549 | QSTVYSDGNTY | 32E01-CDRL1 amino |
| | | acid sequence (IMGT) |
| 550 | KVSNRDS | 32E01-CDRL2 amino |
| | | acid sequence (Kabat) |
| 551 | KVS | 32E01-CDRL2 amino |
| | | acid sequence (IMGT) |
| 552 | MQGTTWPTT | 32E01-CDRL3 amino |
| | | acid sequence (Kabat) |
| 553 | MQGTTWPTT | 32E01-CDRL3 amino |
| | | acid sequence (IMGT) |
| 554 | CAGGTCCAGCTTGTGCAGTCTGGGGCTGAGGTGAGGAAGCCTGGGGC | 35A06-Heavy chain |
| | CTCAGTGAAGGTTTCCTGCAAGGCTTCTGGATACACCTTCACTGACTA | nucleotide sequence |
| | TGCACTACATTGGGTGCGCCAGGCCCCCGGACAAAGGCTTGAGTGGAT | |
| | GGGATGGATCATCGTTGGCAATGGTAAGACAAGATATGCACAGAAGTT | |
| | CCAGGGCAGAGTCACCATTACCTGGGAAACATCCGCGAGCACAGCCTT | |
| | CATGGACCTGAGTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCGTCTCCTCAG | |
| 555 | QVQTVQSGAEVRKPGASVKVSCKASGYTFTDYATHVVVRQAPGQRTEWM | 35A06-Heavy chain |
| | GWTTVGNGKTRYAQKFQGRVTTTTNETSASTAFMDTSSTRSEDTAVYYCA | amino acid sequence |
| | RGFTNTVRGAPYYDGTDVVVGQGTTVTNSS | |
| 556 | GATATTGTGATGACTCAGTCTCCACTCTCCCTGCCCGTCACCCCTGGA | 35A06-Light chain |
| | GAGCCGGCCTCCATCTCCTGCAGGTCTAGTCAGAGCCTCCTGTATAGT | nucleotide sequence |
| | ACTGGATACAACTATTTGGATTGGTACCTGCAGAAGCCAGGGCTGTCT | |
| | CCACAGCTCCTGATCTATTTGGGTTCTAATCGGGCCTCCGGGGTCCCT | |
| | GACAGGTTCAGTGGCAGTGGATCAGGCACAGATTTTACACTGAAAATC | |
| | AGCAGAGTGGAGGCTGAGGATGTTGGGGTTTATTACTGCATGCAAGC | |
| | TCTACAAACTCCCACTTTCGGCGGAGGGACCAAGGTGGAGATCAAAC | |
| 557 | DTVMTQSPTSTPVTPGEPASTSCRSSQSTTYSTGYNYTDWYTQKPGTSPQ | 35A06-Light chain |
| | TTTYTGSNRASGVPDRFSGSGSGTDFTTKTSRVEAEDVGVYYCMQATQTP | amino acid sequence |
| | TFGGGTKVETK | |
| 558 | DYATH | 35A06-CDRH1 amino |
| | | acid sequence (Kabat) |
| 559 | GYTFTDYA | 35A06-CDRH1 amino |
| | | acid sequence (IMGT) |
| 560 | WTTVGNGKTRYAQKFQG | 35A06-CDRH2 amino |
| | | acid sequence (Kabat) |
| 561 | TTVGNGKT | 35A06-CDRH2 amino |
| | | acid sequence (IMGT) |
| 562 | GFTMVRGAPYYDGTDV | 35A06-CDRH3 amino |
| | | acid sequence (Kabat) |
| 563 | ARGFTMVRGAPYYDGTDV | 35A06-CDRH3 amino |
| | | acid sequence (IMGT) |
| 564 | RSSQSTTYSTGYNYTD | 35A06-CDRL1 amino |
| | | acid sequence (Kabat) |
| 565 | QSTTYSTGYNY | 35A06-CDRL1 amino |
| | | acid sequence (IMGT) |
| 566 | TGSNRAS | 35A06-CDRL2 amino |
| | | acid sequence (Kabat) |
| 567 | TGS | 35A06-CDRL2 amino |
| | | acid sequence (IMGT) |
| 568 | MQATQTPT | 35A06-CDRL3 amino |
| | | acid sequence (Kabat) |
| 569 | MQATQTPT | 35A06-CDRL3 amino |
| | | acid sequence (IMGT) |
| 570 | GAGGTGCAGCTGGTGGAGTCTGGGGGAGGCCTGGTCAAGCCTGGGG | 35A10-Heavy chain |
| | GGTCCCTGAGACTCTCCTGTGCAGCCTCTGGATTCACCTTCAGTACCT | nucleotide sequence |
| | ATAGCATGAACTGGGTCCGCCAGGCTCCAGGGAAGGGGCTGGAGTGG | |
| | GTCTCATCCATTAGTCGTAGTAGTAGTTACATATACTACGCAGACTCA | |
| | GTGAAGGGCCGATTCACCATCTCCAGAGACAACGCCAAGAACTCACTG | |
| | TCTCTGCAAATGAACAGCCTGAGAGCCGAGGACACAGCTGTGTATTAC | |
| | TGTGCGAGAGGAGCAGCAGCTGGTACGGACTACTGGGGCCAGGGAAC | |
| | CCTGGTCACCGTCTCCTCAG | |
| 571 | EVQTVESGGGTVKPGGSTRTSCAASGFTFSTYSMNWVRQAPGKGTEWV | 35A10-Heavy chain |
| | SSTSRSSSYTYYADSVKGRFTTSRDNAKNSTSTQMNSTRAEDTAVYYCAR | amino acid sequence |
| | GAAAGTDYWGQGTTVTVSS | |
| 572 | GATATTGTAATGACTCAGTCTCCACTCTCCCTGCCCGTCACCCTTGGAC | 35A10-Light chain |
| | AGCCGGCCTCCATCTCCTGCAGGTCTAGTCAAAGCCTCATATACAGTG | nucleotide sequence |
| | ATGGAAACACCTACTTGAATTGGTTTCAGCAGAGGCCAGGCCAATCTC | |
| | CAAGGCGCCTAATTTATAAGGTTTCTAACCGGGACTCTGGGGTCCCAG | |
| | ACAGATTCAGCGGCAGTGGGTCAGGCACTGATTTCACACTGAAAATCA | |
| | GCAGGGTGGAGGCTGAAGATGTTGGAGTTTATTACTGCTTGCAAGGT | |
| | ACTCTCTGGCCGATCACCTTCGGCCAAGGGACACGACTGGAGATCAAA | |
| | C | |
| 573 | DTVMTQSPTSTPVTTGQPASTSCRSSQSTTYSDGNTYTNWFQQRPGQSPR | 35A10-Light chain |
| | RTTYKVSNRDSGVPDRFSGSGSGTDFTTKTSRVEAEDVGVYYCTQGTTWP | amino acid sequence |
| | TTFGQGTRTETK | |
| 574 | TYSMN | 35A10-CDRH1 amino |
| | | acid sequence (Kabat) |
| 575 | GFTFSTYS | 35A10-CDRH1 amino |
| | | acid sequence (IMGT) |
| 576 | STSRSSSYTYYADSVKG | 35A10-CDRH2 amino |
| | | acid sequence (Kabat) |
| 577 | TSRSSSYT | 35A10-CDRH2 amino |
| | | acid sequence (IMGT) |
| 578 | GAAAGTDY | 35A10-CDRH3 amino |
| | | acid sequence (Kabat) |
| 579 | ARGAAAGTDY | 35A10-CDRH3 amino |
| | | acid sequence (IMGT) |
| 580 | RSSQSTTYSDGNTYTN | 35A10-CDRL1 amino |
| | | acid sequence (Kabat) |
| 581 | QSTTYSDGNTY | 35A10-CDRL1 amino |
| | | acid sequence (IMGT) |
| 582 | KVSNRDS | 35A10-CDRL2 amino |
| | | acid sequence (Kabat) |
| 583 | KVS | 35A10-CDRL2 amino |
| | | acid sequence (IMGT) |
| 584 | TQGTTWPTT | 35A10-CDRL3 amino |
| | | acid sequence (Kabat) |
| 585 | TQGTTWPIT | 35A10-CDRL3 amino |
| | | acid sequence (IMGT) |
| 586 | CAGGTCCAGCTTGTGCAGTCTGGGGCTGAGGTGAGGAAGCCTGGGGC | 35E11-Heavy chain |
| | CTCAGTGAAGGTTTCCTGCAAGGCTTCTGGATACACCTTCACTGACTA | nucleotide sequence |
| | TGCAATACATTGGGTGCGCCAGGCCCCCGGACAAAGGCTTGAGTGGAT | |
| | GGGATGGATCATCGTTGGCAATGGTAAGACAAGATATGCACAGAAGTT | |
| | GCAGGGCAGAGTCACCATTACCTGGGAAACATCCGCGAGCACAGCCTA | |
| | CATGGACCTGACTAGTCTGAGATCGGAAGACACGGCTGTTTATTACTG | |
| | TGCGAGAGGGTTTACTATGGTTCGGGGAGCCCCCTATTATGACGGTTT | |
| | GGACGTCTGGGGCCAAGGGACCACGGTCACCGTCTCCTCAG | |
| 587 | QVQTVQSGAEVRKPGASVKVSCKASGYTFTDYATHVVVRQAPGQRTEWM | 35E11-Heavy chain |
| | GWTTVGNGKTRYAQKTQGRVTTTWETSASTAYMDTTSTRSEDTAVYYCA | amino acid sequence |
| | RGFTMVRGAPYYDGTDVVVGQGTTVTVSS | |
| 588 | GATATTGTGATGACTCAGTCTCCACTCTCCCTGCCCGTCACCCCTGGA | 35E11-Light chain |
| | GAGCCGGCCTCCATCTCCTGCAGGTCTAGTCAGAGCCTCCTGTATAGT | nucleotide sequence |
| | ACTGGATACAACTATTTGGATTGGTACCTGCAGAAGCCAGGGCTGTCT | |
| | CCACATCTCCTGATCTATTTGGGTTCTAATCGGGCCTCCGGGGTCCCT | |
| | GACAGGTTCAGTGGCAGTGGATCAGGCACAGATTTTACACTGAAAATC | |
| | AGCAGAGTGGAGGCTGAGGATGTTGGGGTTTATTACTGCATGCAAGC | |
| | TCTACAAACTCCCATTTTCGGCGGAGGGACCAAGGTGGAGATCAAAC | |
| 589 | DTVMTQSPTSTPVTPGEPASTSCRSSQSTTYSTGYNYTDWYTQKPGTSPH | 35E11-Light chain |
| | TTTYTGSNRASGVPDRFSGSGSGTDFTTKTSRVEAEDVGVYYCMQATQTP | amino acid sequence |
| | TFGGGTKVETK | |
| 590 | DYATH | 35E11-CDRH1 amino |
| | | acid sequence (Kabat) |
| 591 | GYTFTDYA | 35E11-CDRH1 amino |
| | | acid sequence (IMGT) |
| 592 | WTTVGNGKTRYAQKTQG | 35E11-CDRH2 amino |
| | | acid sequence (Kabat) |
| 593 | TTVGNGKT | 35E11-CDRH2 amino |
| | | acid sequence (IMGT) |
| 594 | GFTMVRGAPYYDGTDV | 35E11-CDRH3 amino |
| | | acid sequence (Kabat) |
| 595 | ARGFTMVRGAPYYDGTDV | 35E11-CDRH3 amino |
| | | acid sequence (IMGT) |
| 596 | RSSQSTTYSTGYNYTD | 35E11-CDRL1 amino |
| | | acid sequence (Kabat) |
| 597 | QSTTYSTGYNY | 35E11-CDRL1 amino |
| | | acid sequence (IMGT) |
| 598 | TGSNRAS | 35E11-CDRL2 amino |
| | | acid sequence (Kabat) |
| 599 | TGS | 35E11-CDRL2 amino |
| | | acid sequence (IMGT) |
| 600 | MQATQTPT | 35E11-CDRL3 amino |
| | | acid sequence (Kabat) |
| 601 | MQATQTPT | 35E11-CDRL3 amino |
| | | acid sequence (IMGT) |
| 602 | ASTKGPSVFPTAPCSRSTSESTAATGCTVKDYFPEPVTVSWNSGATTSGV | IgG4 heavy chain |
| | HTFPAVTQSSGTYSTSSVVTVPSSSTGTKTYTCNVDHKPSNTKVDKRVES | constant region IgG4- |
| | KYGPPCPPCPAPEFEGGPSVFTFPPKPKDTTMTSRTPEVTCVVVDVSQEDP | PE |
| | EVQFNVVYVDGVEVHNAKTKPREEQFNSTYRVVSVTTVTHQDWTNGKEY | |
| | KCKVSNKGTPSSTEKTTSKAKGQPREPQVYTTPPSQEEMTKNQVSTTCTV | |
| | KGFYPSDTAVEWESNGQPENNYKTTPPVTDSDGSFFTYSRUTVDKSRWQ | |
| | EGNVFSCSVMHEATHNHYTQKSTSTSTGK | |

1. An antibody or fragment thereof which binds to human Nav1.7 (Seq ID No:2) and comprises a VH region which comprises:
a. a CDRH3 sequence of 22D04 selected from SEQ ID Nos: 338 and 339;
b. a CDRH3 sequence of 22G08 selected from SEQ ID Nos: 354 and 355;
c. a CDRH3 sequence of 22G09 selected from SEQ ID Nos: 370 and 371;
d. a CDRH3 sequence of 25A01 selected from SEQ ID Nos: 386 and 387;
e. a CDRH3 sequence of 25C01 selected from SEQ ID Nos: 402 and 403;
f. a CDRH3 sequence of 25F08 selected from SEQ ID Nos: 418 and 419;
g. a CDRH3 sequence of 28B08 selected from SEQ ID Nos: 450 and 451;
h. a CDRH3 sequence of 28C11 selected from SEQ ID Nos: 466 and 467;
i. a CDRH3 sequence of 32B04 selected from SEQ ID Nos: 514 and 515;
j. a CDRH3 sequence of 32D04 selected from SEQ ID Nos: 530 and 531;
k. a CDRH3 sequence of 35A06 selected from SEQ ID Nos: 562 and 563; or
l. a CDRH3 sequence of 35E11 selected from SEQ ID Nos: 594 and 595.

2. The antibody or fragment according to claim 1, wherein:
a. for part a), the antibody or fragment further comprises a CDRH1 sequence of 22D04 selected from SEQ ID Nos: 334 and 335;
b. for part b), the antibody or fragment further comprises a CDRH1 sequence of 22G08 selected from SEQ ID Nos: 350 and 351;
c. for part c), the antibody or fragment further comprises a CDRH1 sequence of 22G09 selected from SEQ ID Nos: 366 and 367;
d. for part d), the antibody or fragment further comprises a CDRH1 sequence of 25A01 selected from SEQ ID Nos: 382 and 383;
e. for part e), the antibody or fragment further comprises a CDRH1 sequence of 25C01 selected from SEQ ID Nos: 398 and 399;
f. for part f), the antibody or fragment further comprises a CDRH1 sequence of 25F08 selected from SEQ ID Nos: 414 and 415;
g. for part g), the antibody or fragment further comprises a CDRH1 sequence of 28B08 selected from SEQ ID Nos: 446 and 447;
h. for part h), the antibody or fragment further comprises a CDRH1 sequence of 28C11 selected from SEQ ID Nos: 462 and 463;
i. for part i), the antibody or fragment further comprises a CDRH1 sequence of 32B04 selected from SEQ ID Nos: 510 and 511;
j. for part j), the antibody or fragment further comprises a CDRH1 sequence of 32D04 selected from SEQ ID Nos: 526 and 527;
k. for part k), the antibody or fragment further comprises a CDRH1 sequence of 35A06 selected from SEQ ID Nos: 558 and 559; or
l. for part l), the antibody or fragment further comprises a CDRH1 sequence of 35E11 selected from SEQ ID Nos: 590 and 591.

3. The antibody or fragment according to claim 1, wherein:
a. for part a), the antibody or fragment further comprises a CDRH2 sequence of 22D04 selected from SEQ ID Nos: 336 and 337;
b. for part b), the antibody or fragment further comprises a CDRH2 sequence of 22G08 selected from SEQ ID Nos: 352 and 353;
c. for part c), the antibody or fragment further comprises a CDRH2 sequence of 22G09 selected from SEQ ID Nos: 368 and 369;
d. for part d), the antibody or fragment further comprises a CDRH2 sequence of 25A01 selected from SEQ ID Nos: 384 and 385;
e. for part e), the antibody or fragment further comprises a CDRH2 sequence of 25C01 selected from SEQ ID Nos: 400 and 401;
f. for part f), the antibody or fragment further comprises a CDRH2 sequence of 25F08 selected from SEQ ID Nos: 416 and 417;
g. for part g), the antibody or fragment further comprises a CDRH2 sequence of 28B08 selected from SEQ ID Nos: 448 and 449;
h. for part h), the antibody or fragment further comprises a CDRH2 sequence of 28C11 selected from SEQ ID Nos: 464 and 465;
i. for part i), the antibody or fragment further comprises a CDRH2 sequence of 32B04 selected from SEQ ID Nos: 512 and 513;
j. for part j), the antibody or fragment further comprises a CDRH2 sequence of 32D04 selected from SEQ ID Nos: 528 and 529;
k. for part k), the antibody or fragment further comprises a CDRH2 sequence of 35A06 selected from SEQ ID Nos: 560 and 561; or
l. for part l), the antibody or fragment further comprises a CDRH2 sequence of 35E11 selected from SEQ ID Nos: 592 and 593.

4. The antibody or fragment according to claim 1, wherein:
a. for part a), the antibody or fragment comprises the VH region of 22D04 (SEQ ID No: 331);
b. for part b), the antibody or fragment comprises the VH region of 22G08 (SEQ ID No: 347);
c. for part c), the antibody or fragment comprises the VH region of 22G09 (SEQ ID No: 363);
d. for part d), the antibody or fragment comprises the VH region of 25A01 (SEQ ID No: 379);
e. for part e), the antibody or fragment comprises the VH region of 25C01 (SEQ ID No: 395);
f. for part f), the antibody or fragment comprises the VH region of 25F08 (SEQ ID No: 411);
g. for part g), the antibody or fragment comprises the VH region of 28B08 (SEQ ID No: 443);
h. for part h), the antibody or fragment comprises the VH region of 28C11 (SEQ ID No: 459);
i. for part i), the antibody or fragment comprises the VH region of 32B04 (SEQ ID No: 507);
j. for part j), the antibody or fragment comprises the VH region of 32D04 (SEQ ID No: 523);
k. for part k), the antibody or fragment comprises the VH region of 35A06 (SEQ ID No: 555); or
l. for part l), the antibody or fragment comprises the VH region of 35E11 (SEQ ID No: 587).

5. The antibody or fragment according to claim 1, which further comprises a VL region which comprises:
a. for part a), a CDRL1 sequence of 22D04 selected from SEQ ID Nos: 340 and 341;
b. for part b), a CDRL1 sequence of 22G08 selected from SEQ ID Nos: 356 and 357;
c. for part c), a CDRL1 sequence of 22G09 selected from SEQ ID Nos: 372 and 373;
d. for part d), a CDRL1 sequence of 25A01 selected from SEQ ID Nos: 388 and 389;
e. for part e), a CDRL1 sequence of 25C01 selected from SEQ ID Nos: 404 and 405;
f. for part f), a CDRL1 sequence of 25F08 selected from SEQ ID Nos: 420 and 421;
g. for part g), a CDRL1 sequence of 28B08 selected from SEQ ID Nos: 452 and 453;
h. for part h), a CDRL1 sequence of 28C11 selected from SEQ ID Nos: 468 and 469;
i. for part i), a CDRL1 sequence of 32B04 selected from SEQ ID Nos: 516 and 517;
j. for part j), a CDRL1 sequence of 32D04 selected from SEQ ID Nos: 532 and 533,
k. for part k), a CDRL1 sequence of 35A06 selected from SEQ ID Nos: 564 and 565; or
l. for part l), a CDRL1 sequence of 35E11 selected from SEQ ID Nos: 596 and 597.

6. The antibody or fragment according to claim 5, which further comprises a VL region which comprises:
a. for part a), a CDRL2 sequence of 22D04 selected from SEQ ID Nos: 342 and 343;
b. for part b), a CDRL2 sequence of 22G08 selected from SEQ ID Nos: 358 and 359;
c. for part c), a CDRL2 sequence of 22G09 selected from SEQ ID Nos: 374 and 375;
d. for part d), a CDRL2 sequence of 25A01 selected from SEQ ID Nos: 390 and 391;
e. for part e), a CDRL2 sequence of 25C01 selected from SEQ ID Nos: 406 and 407;
f. for part f), a CDRL2 sequence of 25F08 selected from SEQ ID Nos: 422 and 423;
g. for part g), a CDRL2 sequence of 28B08 selected from SEQ ID Nos: 454 and 455;
h. for part h), a CDRL2 sequence of 28C11 selected from SEQ ID Nos: 470 and 471,
i. for part i), a CDRL2 sequence of 32B04 selected from SEQ ID Nos: 518 and 519;
j. for part j), a CDRL2 sequence of 32D04 selected from SEQ ID Nos: 534 and 535;
k. for part k), a CDRL2 sequence of 35A06 selected from SEQ ID Nos: 566 and 567; or
l. for part 1), a CDRL2 sequence of 35E11 selected from SEQ ID Nos: 598 and 599.

7. The antibody or fragment according to claim 5, which further comprises a VL region which comprises:
a. for part a), a CDRL3 sequence of 22D04 selected from SEQ ID Nos: 344 and 345;
b. for part b), a CDRL3 sequence of 22G08 selected from SEQ ID Nos: 360 and 361;
c. for part c), a CDRL3 sequence of 22G09 selected from SEQ ID Nos: 376 and 377;
d. for part d), a CDRL3 sequence of 25A01 selected from SEQ ID Nos: 392 and 393;
e. for part e), a CDRL3 sequence of 25C01 selected from SEQ ID Nos: 408 and 409;
f. for part f), a CDRL3 sequence of 25F08 selected from SEQ ID Nos: 424 and 425;
g. for part g), a CDRL3 sequence of 28B08 selected from SEQ ID Nos: 456 and 457;
h. for part h), a CDRL3 sequence of 28C11 selected from SEQ ID Nos: 472 and 473,
i. for part i), a CDRL3 sequence of 32B04 selected from SEQ ID Nos: 520 and 521;
j. for part j), a CDRL3 sequence of 32D04 selected from SEQ ID Nos: 536 and 537;
k. for part k), a CDRL3 sequence of 35A06 selected from SEQ ID Nos: 568 and 569; or
l. for part l), a CDRL3 sequence of 35E11 selected from SEQ ID Nos: 600 and 601.

8. The antibody or fragment according to claim 4, wherein:
a. for part a), the antibody or fragment comprises the VL region of 22D04 (SEQ ID No: 333);
b. for part b), the antibody or fragment comprises the VL region of 22G08 (SEQ ID No: 349);
c. for part c), the antibody or fragment comprises the VL region of 22G09 (SEQ ID No: 365);
d. for part d), the antibody or fragment comprises the VL region of 25A01 (SEQ ID No: 381);
e. for part e), the antibody or fragment comprises the VL region of 25C01 (SEQ ID No: 397);
f. for part f), the antibody or fragment comprises the VL region of 25F08 (SEQ ID No: 413);
g. for part g), the antibody or fragment comprises the VL region of 28B08 (SEQ ID No: 445);
h. for part h), the antibody or fragment comprises the VL region of 28C11 (SEQ ID No: 461);
i. for part i), the antibody or fragment comprises the VL region of 32B04 (SEQ ID No: 509);
j. for part j), the antibody or fragment comprises the VL region of 32D04 (SEQ ID No: 525);
k. for part k), the antibody or fragment comprises the VL region of 35A06 (SEQ ID No: 557); or
l. for part l), the antibody or fragment comprises the VL region of 35E11 (SEQ ID No: 589).

9. (canceled)

10. The antibody or fragment according to claim 1, which has an isotype selected from IgG1, IgG2, IgG2a, IgG2c, IgG3 and IgG4.

11. The antibody or fragment according to claim 1 which further comprises a heavy chain constant region which is IgG4-PE (Seq ID No: 602).

12. The antibody or fragment which binds to the same epitope of human Nav1.7 (SEQ ID No:2) as an antibody as defined in claim 1.

13. The antibody or fragment which competes for binding to human Nav1.7 (SEQ ID No:2) with an antibody as defined in claim 8, optionally as measured by SPR or ELISA.

14. (canceled)

15. (canceled)

16. (canceled)

17. (canceled)

18. (canceled)

19. (canceled)

20. (canceled)

21. (canceled)

22. (canceled)

23. The antibody or fragment according to claim 1, wherein the antibody or fragment has an affinity (Kd) of less than 100 nM and optionally wherein the affinity is measured using an SPR method on a NAV1.7 polypeptide, wherein the SPR method comprises the following steps:
a. Coupling anti-mouse IgG to a biosensor chip such as by primary amine coupling;
b. Exposing the anti-mouse IgG to a test IgG antibody to capture test antibody on the chip;
c. Passing the test antigen over the chip's capture surface at 1024 nM, 256 nM, 64 nM, 16 nM, 4 nM with a 0 nM; and
d. And determining the affinity of binding of test antibody to test antigen using surface plasmon resonance, e.g., at 25° C. or at 37° C., optionally in physiological buffer such as a buffer at pH7.6, comprising 150 mM NaCl, 0.05% detergent and 3 mM EDTA, or a buffer containing 10 mM Hepes, or a buffer which is EMS-EP.

24. (canceled)

25. An antibody fragment according to claim 1, wherein the fragment is selected from a Fab, a Fab′, a F(ab′)2, a bispecific Fab, a dsFv, a camelized VH, a bispecific scFv, a diabody, a triabody and a scFv.

26. The antibody or fragment according to claim 1 which is monoclonal.

27. (canceled)

28. The nucleic acid encoding an antibody or fragment as defined in claim 1.

29. The nucleic acid that encodes a VH domain and/or a VL domain of an antibody or fragment as defined in claim 8.

30. (canceled)

31. (canceled)

32. A vector comprising the nucleic acid as defined claim 29.

33. A host cell comprising the nucleic acid as defined in claim 29.

34. (canceled)

35. (canceled)

36. The pharmaceutical composition comprising an antibody or fragment as defined in claim 1, and a diluent, excipient or carrier.

37. (canceled)

38. (canceled)

39. (canceled)

40. (canceled)

41. (canceled)

42. (canceled)

43. A method of treating and/or preventing and/or reducing the risk of a NAV1.7-mediated disease or condition in a human by administering to said human a therapeutically effective amount of an antibody or fragment as defined in claim 1.

44. (canceled)

45. The method according to claim 43, wherein the NAV1.7-mediated disease or condition is selected from painful diabetic neuropathy, post-herpetic neuropathy, trigeminal neuralgia, osteoarthritis, chronic back pain, nerve compression pain, sciatic nerve compression, cancer pain, migraine, post-operative pain, and fibromyalgia.

46. A method of generating an antibody against a NAV protein of interest comprising the steps of:
a. immunising a non-human mammal with MEF or HEK cells which express said NAV protein of interest on its surface; and
b. immunising said non-human mammal with one or two fragment(s) of said NAV protein of interest;
c. immunising said non-human mammal with MEF or HEK (e.g. HEK) cells which express said NAV protein of interest on its surface; and
d. immunising said non-human mammal with said one or two fragment(s) of the NAV protein of interest.

47. (canceled)

48. The method according to claim 46, wherein the non-human animal has, before each immunisation, been dosed with a compound which stabilises said NAV protein of interest in an open, closed or activated conformation, in an amount sufficient to stabilise said NAV protein of interest in said open conformation, optionally wherein the compound is selected from tetrodotoxin (TTX), saxitoxin (STX), hanatoxin, centipede toxin, μ-SLPTX-Ssm6a, Protoxin-I (ProTx-I), Protoxin-II (ProTx-II), Huwentoxin-IV (HwTx-IV) and a conotoxin (such as μ-GIIIA, μ-GIIIB, μ-GIIIC, μ-PIIIA, μ-TIIIA, μ-SmIIIA, μKIIIA, μ-SIIIA, μ-CoIIIB, μ-CIIIA, μ-MIIIA, μO-MrVIA, μO-MrVIB, δ-TxVIA, δ-TxVIB, δGmVIA, δ-PVIA, δ-NgVIA, δ-EVIA and δ-SVIE, in particular μ-KIIIA).

49. The antibody or fragment according to claim 2, wherein:
a. for part a), the antibody or fragment further comprises a CDRH2 sequence of 22D04 selected from SEQ ID Nos: 336 and 337;
b. for part b), the antibody or fragment further comprises a CDRH2 sequence of 22G08 selected from SEQ ID Nos: 352 and 353;
c. for part c), the antibody or fragment further comprises a CDRH2 sequence of 22G09 selected from SEQ ID Nos: 368 and 369;
d. for part d), the antibody or fragment further comprises a CDRH2 sequence of 25A01 selected from SEQ ID Nos: 384 and 385;
e. for part e), the antibody or fragment further comprises a CDRH2 sequence of 25C01 selected from SEQ ID Nos: 400 and 401;
f. for part f), the antibody or fragment further comprises a CDRH2 sequence of 25F08 selected from SEQ ID Nos: 416 and 417;
g. for part g), the antibody or fragment further comprises a CDRH2 sequence of 28B08 selected from SEQ ID Nos: 448 and 449;
h. for part h), the antibody or fragment further comprises a CDRH2 sequence of 28C11 selected from SEQ ID Nos: 464 and 465;
i. for part i), the antibody or fragment further comprises a CDRH2 sequence of 32B04 selected from SEQ ID Nos: 512 and 513;
j. for part j), the antibody or fragment further comprises a CDRH2 sequence of 32D04 selected from SEQ ID Nos: 528 and 529;
k. for part k), the antibody or fragment further comprises a CDRH2 sequence of 35A06 selected from SEQ ID Nos: 560 and 561; or
l. for part l), the antibody or fragment further comprises a CDRH2 sequence of 35E11 selected from SEQ ID Nos: 592 and 593.

50. The antibody or fragment according to claim 6, which further comprises a VL region which comprises:
a. for part a), a CDRL3 sequence of 22D04 selected from SEQ ID Nos: 344 and 345;
b. for part b), a CDRL3 sequence of 22G08 selected from SEQ ID Nos: 360 and 361,
c. for part c), a CDRL3 sequence of 22G09 selected from SEQ ID Nos: 376 and 377;
d. for part d), a CDRL3 sequence of 25A01 selected from SEQ ID Nos: 392 and 393,
e. for part e), a CDRL3 sequence of 25C01 selected from SEQ ID Nos: 408 and 409;
f. for part f), a CDRL3 sequence of 25F08 selected from SEQ ID Nos: 424 and 425;
g. for part g), a CDRL3 sequence of 28B08 selected from SEQ ID Nos: 456 and 457;
h. for part h), a CDRL3 sequence of 28C11 selected from SEQ ID Nos: 472 and 473;
i. for part i), a CDRL3 sequence of 32B04 selected from SEQ ID Nos: 520 and 521;
j. for part j), a CDRL3 sequence of 32D04 selected from SEQ ID Nos: 536 and 537;
k. for part k), a CDRL3 sequence of 35A06 selected from SEQ ID Nos: 568 and 569; or
l. for part l), a CDRL3 sequence of 35E11 selected from SEQ ID Nos: 600 and 601.
